(19) United States
(12) Patent Application Publication
(10) Pub. No.: US 2021/0260113 A1
(43) Pub. Date: Aug. 26, 2021

(54) DEVICES, METHODS, COMPOSITIONS AND SYSTEMS FOR THE TREATMENT OF AGING AND AGE-RELATED DISORDERS

(71) Applicant: Gero LLC, Moscow (RU)

(72) Inventors: Maksim Nikolaevich KHOLIN, Moscow (RU); Petr Olegovich FEDICHEV, Dolgoprudny (RU); Olga Andreevna BURMISTROVA, Reutov (RU); Konstantin Alexandrovich AVKHACHEV, Moscow (RU); Dmitry Veniaminovich SHISHOV, Verkhovashie (RU); Andrey Evgenievich TARKHOV, Syktyvkar (RU)

(73) Assignee: Gero LLC, Moscow (RU)

(21) Appl. No.: 17/150,466

(22) Filed: Jan. 15, 2021

Related U.S. Application Data
(63) Continuation-in-part of application No. PCT/RU2019/050115, filed on Jul. 17, 2019.

(30) Foreign Application Priority Data
Jul. 17, 2018 (RU) 2018126402

(51) Int. Cl. A61K 35/16; C07K 16/28; C07K 16/38; C07K 16/22; C07K 16/18; A61P 39/00

(52) U.S. Cl. CPC A61K 35/16; C07K 16/2833; C07K 16/2869; C07K 16/38; A61P 39/00; C07K 16/22; C07K 16/18; C07K 16/2896; C07K 16/2878

(57) ABSTRACT

The present disclosure provides methods, devices, kits, and agents for the treatment of aging and aging related diseases and disorders and related systems and methods.

### CROSS-REFERENCE TO RELATED APPLICATIONS

This application is a continuation-in-part (CIP) of PCT Application No. PCT/RU2019/050115 filed on Jul. 17, 2019, which claims priority to Russian Patent Application No. RU2018126402 filed on Jul. 17, 2018, the contents of which are incorporated herein by reference in their entireties.

### BACKGROUND

The development of methods, devices and systems for increasing health span or treating aging or age-related diseases would benefit many. One promising avenue of anti-aging research is concerned with infusing “aged” or “old” mice with the blood of younger donors. The infusion of “young” blood has been shown to improve the health of aged mice and, in some cases, treat age-related diseases. Studies for human application are ongoing, and some evidence of anti-aging effects in humans started to come, however, applying this technique would require extensive collection of blood from young and healthy donors. Given the already high demand of blood in modern medicine, mass collection of blood may be difficult or impossible and may have some ethical issues. Blood transfusions from donors also carry inherent risks such as the spread of disease, nonhemolytic fever and other side effects. Therefore, methods, devices and systems that aid in the transformation of a patient's own aged blood into blood having medical characteristics of younger blood, would be beneficial.

The removal, inhibition, modulation, or deactivation of specific proteins in the blood can improve health. Many of such evidence relates to young plasma studies and parabiosys studies. For example, in [Villeda S A, Luo J, Mosher K I, Zou B, Britschgi M, Bieri G, Stan T M, Fainberg N, Ding Z, Eggel A, Lucin K M, Czirr E, Park J S, Couillard-Després S, Aigner L, Li G, Peskind E R, Kaye J A, Quinn J F, Galasko D R, Xie X S, Rando T A, Wyss-Coray T. The ageing systemic milieu negatively regulates neurogenesis and cognitive function. Nature. 2011; 477(7362):90-4] using heterochronic parabiosis it is demonstrated that blood-borne factors present in the systemic milieu can inhibit or promote adult neurogenesis in an age-dependent fashion in mice. Heterochronic parabiosis rejuvenates the performance of old tissue stem cells at some expense to the young, but whether this is through shared circulation or shared organs is unclear. In [Rebo J, Mehdipour M, Gathwala R, Causey K, Liu Y, Conboy M J, Conboy I M. A single heterochronic blood exchange reveals rapid inhibition of multiple tissues by old blood. Nat Commun. 2016 Nov. 22; 7:13363] authors show that heterochronic blood exchange between young and old mice without sharing other organs, affects tissues within a few days, and leads to different outcomes than heterochronic parabiosis.

It has been demonstrated that such blood-borne factors can inhibit or promote adult neurogenesis in an age-dependent fashion in mice. In many cases, the inhibitory effects of old blood are more pronounced than the benefits of young blood. It was found that any injury sustained to peripheral tissue compounds the negative effects of the old blood. In contrast, young blood can induce vascular remodeling and increase neurogenesis and olfactory discrimination in aging mice. Further, it has been shown that the increased concentrations GDF11 present in young blood can improve the cerebral vasculature and enhance neurogenesis. Young blood also enhances synaptic plasticity, eliciting an increase in the expression of immediate early genes and the density of dendritic spines as well as enhancements in long term potentiation. At a cognitive level, long-term administration of young plasma over one month is sufficient to reverse cognitive impairments in hippocampal-dependent learning and memory in old mice through increased activation of the transcription factor cAMP response element binding protein.

Changes in Alzheimer's disease-related pathology were first investigated in parabiosis studies in which young transgenic mice carrying APPswe/PS1dE9 mutations were joined with age-matched young wild-type animals. Parabiosis during 6 months significantly reduced brain Δβ burden without alterations in the expression of amyloid precursor protein, Δβ generating and degrading enzymes, Δβ transport receptors, and AD-type pathologies including hyperphosphorylated tau, neuroinflammation, as well as neuronal degeneration and loss in the brains of parabiotic AD mice. While this was not a direct effect of rejuvenation, as all parabionts were age-matched isochronic pairs, it does suggest a significant role for the systemic environment in the progression of neurodegenerative disease.

Mice studies show that to reach a considerable anti-aging effect almost all plasma should be changed in mice for the young plasma. In humans the procedure may require at least a few hours with some time between every such procedure. An alternative to blood changing is the removal, reduction or deactivation of “pro-aging” elements from blood or plasma of the patient. For example, Irina Conboy Berkeley team is working on the device for selective removal from blood the protein they consider “pro-aging” (Scientists to ‘reset’ blood proteins in attempt to slow ageing process, The Guardian, 22 Nov. 2016).

However, selective removal of proteins and biomarkers from blood and plasma remains non-trivial. Many proteins and biomarkers, such as testosterone for example, naturally change with age. Other elements are required for general health and should not be removed or reduced. Selected removal, reduction or deactivation of target proteins or biomarkers related to aging or age-related diseases has a positive health effect on subjects in need of such treatment, e.g. as shown in Can an Apheresis Therapy become an Effective Method for Anti-Aging Medicine?, Hiroshi Miyamoto, Yukihiko Nosé Sep. 1, 2010, Anti-Aging Medicine 7 (9):100-106, 2010 (c) Japanese Society of Anti-Aging Medicine)

There remains a need for devices and methods for the selective removal, reduction or deactivation of target proteins and biomarkers in the blood.

Gdf-15 antibodies are suggested for several indications, e.g. described in application WO2014100689A1; any of gdf-15 antibodies described in application WO2015144855A1, any of gdf-15 antibodies described in Application US20180142013A1, any of gdf-15 antibodies described in application US20170306008A1, any of the agents described in application KR20180030898A, WO2017189724A1, U.S. Pat. No. 9,175,076B2, US20170137506A1, AV-380 (a humanized Immunoglobulin G 1 (IgG1) inhibitory monoclonal antibody) licensed by AVEO Oncology (NASDAQ: AVEO) to Novartis. Gdf-15 binding particle is suggested in WO2017004159A1.

In U.S. Pat. No. 8,895,011B2 provided are a means for the prevention and treatment of obesity and/or insulin resistance and, particularly, pharmaceutical drugs for the treatment of obesity and/or insulin resistance under the influence of Follistatin-like 3 (FSTL3). Specifically, provided is an insulin resistance improving drug comprising an FSTL3 inhibitor as an active ingredient, particularly, the insulin resistance improving drug, wherein the FSTL3 inhibitor is one of (A) a substance specifically binding to FSTL3 to inhibit or suppress a function of FSTL3, (B) an inhibitor for expression of FSTL3, and (C) a competitor of FSTL3.

In US20170298128A1 suggested an invention of methods and compositions containing an antagonist of a negative regulator of GDF-11 (e.g., an antibody) for use in treating an age-related condition (FSTL3 being one of these negative regulators). In particular the methods and compositions can be used to treat an age related cardiovascular condition such as diastolic heart failure. A method of treating an age-related condition in a subject in need thereof, the method comprising administering an effective amount of an antagonist of an endogenous negative regulator of GDF-11 activity to the subject, thereby ameliorating at least one symptom of the age-related condition in the subject, wherein the antagonist is an anti-FSTL3 antibody

In WO2017004159 the particles targeting P2 microglobulin (B2M) are suggested. The agent may be an anti-B2M antibody. Particles targeting B2M may be useful for treating or preventing memory loss, cognitive decline, peripheral arterial disease, dialysis-related amyloidosis, chronic lymphocytic leukaemia, multiple myeloma, and lymphoma, in addition to other diseases and conditions.

The role of B2M in aging is mentioned in A single heterochronic blood exchange reveals rapid inhibition of multiple tissues by old blood (Rebo et al. Nature Communications, 2016). Lixelle™ Beta 2-microglobulin Apheresis Column Kaneka (Osaka, Japan) is known, but to our knowledge no devices are known to selectively bind B2M and at least one other of the Targets as defined below.

The particle binding BMP4 is suggested in WO2017004159A1.

Anti-FAS antibodies are suggested in U.S. Pat. No. 6,086,877A as Therapeutic agent for rheumatic disease. Various studies were made about applications of the anti-Fas antibody including a therapeutic agent for AIDS and tumors (Japanese Patent Publication No. 2-237935; WO 91/10448). Some anti-FAS binding elements are also known, e.g. APO010, APO 010, mega FasLigand Apoxis S. A., Bio-Link, Oncology Venture, Topotarget A S Intravenous, APO010 is a synthetic, recombinant, soluble, hexameric fusion protein consisting of three human Fas ligand (FasL) extracellular domains fused to the dimer-forming collagen domain of human adiponectin with potential pro-apoptotic and antineoplastic activities. Assembled into a soluble hexameric structure mimics the ligand clustering of endogenous active FasL, Fas receptor agonist APO010 activates the Fas receptor, resulting in caspase-dependent apoptosis in susceptible tumor cell populations. FasL is a transmembrane protein of the tumor necrosis factor (TNF) superfamily and a pro-apoptotic ligand for the death receptor Fas. APO010 is indicated for the treatment of advanced solid tumors.

DE098 of Centocor, Inc., Argenes Inc, Santen Pharmaceutical Co Ltd (other names) ARG 098, ARG098, DE 098 DE098 contains fas monoclonal antibody as an active ingredient. Fas monoclonal antibody is an immunoglobulin that acts by binding to and inhibits the actions of cluster of differentiation 95 receptor, cellular surface receptor molecules inducing apoptosis. After anti-Fas antibodies bind to Fas molecules, signals are transmitted to cells, and apoptosis of synovial cells is induced within damaged joints, thereby inhibiting their abnormal proliferation. DE098 is indicated for the treatment of rheumatoid arthritis.

F45D9 mAb F45D9 of IMED AB company. F45D9 is indicated for the treatment of graft-versus-host disease, human immunodeficiency virus infection and ischemic reperfusion injury. F61F12mAb F61F12 of IMED AB company. F61F12 is an agonistic fully human anti-Fas monoclonal antibody being developed for the treatment of cancer. Fsn1103 of Fusion Therapeutics Fsn1103 was under development for the treatment of cancer using Fusion Expression Technology (FET). Myocardial Infarction Therapy SERVIER of CNRS—Centre national de la recherche scientifique, Les Laboratoires Servier. Myocardial Infarction Therapy acts by inhibiting the apoptotic process that leads to irreversible myocardial lesions. It inhibits the interaction of Fas receptor with Daxx and FADD leads to a strong decrease of the apoptosis of cardiac cells after myocardial infarction. Myocardial Infarction Therapy is indicated for the treatment of myocardial ischaemia.

Novotarg of Celonic GmbH, Baliopharm A G Novotarg is a bispecific antibody targeting cluster of differentiation 20 (CD20) and cluster of differentiation 95 (CD95). Novotarg acts as an agonist to CD95, which is a death receptor and mediates apoptosis in the cells expressing CD20. Novotarg is indicated for the treatment of B-cell malignancies like B-cell lymphoma and chronic lympholytic leukemia and B-cell mediated autoimmune and inflammatory disorders.

PB102 is a chimeric monoclonal anti-CD55 antibody developed by Percipio Biotherapeutics, Inc for use as a monotherapy, in combination with existing therapeutics (e.g. Rituxan), or as a vehicle for cancer-specific toxin delivery.

### SUMMARY

Disclosed are protein binding devices, pharmaceutical compositions, and methods of providing an anti-aging treatment or of treating or preventing an age-related disease or disorder. In one aspect, a protein binding device is disclosed. The protein binding device comprises a housing, wherein the housing defines a lumen and the housing further comprises an inlet port for receiving fluid to the lumen, and an outlet port for releasing fluid from the lumen, wherein the lumen comprises binding elements. The binding elements selectively bind a protein selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In another aspect, a pharmaceutical composition is disclosed. The pharmaceutical composition is provided as part of an anti-aging treatment or for treating or preventing an age-related disease or disorder. The pharmaceutical composition comprises an agent configured to bind to, inhibit, or degrade a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB. The pharmaceutical composition also comprises at least one pharmaceutically acceptable excipient.

In some aspects, the protein of the pharmaceutical composition is FSTL3. In these aspects, the pharmaceutical composition is devoid of or excludes compounds or molecules for treating insulin resistance, diastolic heart failure, and obesity.

In some aspects, the agent of the pharmaceutical composition is selected from the group consisting of: a protein, a polymer, an aptamer, a SOMAmer, a peptide, a virus, a small molecule, a nanoparticle, an antibody, a monoclonal antibody, a polyclonal antibody, a humanized monoclonal antibody, a human monoclonal antibody, a human or humanized polyclonal antibody, an anti-GDF15 human antibody, an anti-FSTL3 human antibody, an anti-BMP4 human antibody, and an anti-FRZB (sFRP3) human antibody.

In certain aspects, the agent is bound to a particle.

Also disclosed is a pharmaceutical composition comprising a blood plasma fraction. The blood plasma fraction can comprise a negligible amount of a protein selected from the group consisting of CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

For example, the negligible amount can be between about 0.008 pg/mL and about 15,000 ng/mL.

The pharmaceutical composition can also comprise one or more biomarkers characteristic of aged blood. For example, one of the biomarkers characteristic of aged blood can be a biological age of the subject from whose blood the pharmaceutical composition is produced, wherein the biological age of the subject is greater than 45 years of age.

Also disclosed is a method of providing an anti-aging treatment or of treating or preventing an age-related disease or disorder of a subject. The method can comprise reducing, inhibiting, or degrading a protein selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in the blood of the subject.

The method can further comprise administering to the subject a gene therapy.

In some aspects, the age-related disease or disorder can be associated with an alleviated level of a protein selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in a bodily fluid of the subject.

In these and other aspects, the age-related disease or disorder can be selected from the group consisting of frailty, Alzheimer's disease, Parkinson's disease, Huntington's diseases, cardiovascular disease, renal failure, muscle wasting or cachexia, osteopenia or osteoporosis, obesity, insulin resistance or diabetes, diverse adult-onset cancers, atherosclerosis, cardiovascular disease, adult cancer, arthritis, cataracts, osteoporosis, type 2 diabetes, hypertension, age-progressive dementia; amyotrophic lateral sclerosis, stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, age related decline in immune response to vaccines, age related decline in response to immunotherapy, myocardial infarction, acute coronary syndrome, sarcopenia, sarcopenic obesity, senile osteoporosis, urinary incontinence, stroke, atrophic gastritis, camptocormia, chronic obstructive pulmonary disease, coronary artery disease, dopamine dysregulation syndrome, late life depression, osteoarthritis, chronic fatigue syndrome, senile dementia, mild cognitive impairment due to aging, Creutzfeldt-Jakob disease, stroke, CNS cerebral senility, pre-diabetes, diabetes, peripheral arterial disease, aortic valve disease, stroke, Lewy body disease, progressive subcortical gliosis, progressive supranuclear palsy, thalamic degeneration syndrome, hereditary aphasia, myoclonus epilepsy, macular degeneration, pressure ulcers, delirium, progressive subcortical gliosis, progressive supranuclear palsy, thalamic degeneration syndrome, hereditary aphasia, myoclonus epilepsy, and metabolic disorder.

In some aspects, the anti-aging treatment can be selected from the group consisting of a treatment leading to prevention, amelioration or lessening the effects of aging; decreasing or delaying an increase in a biological age of the subject; slowing a rate of aging of the subject; prevention, amelioration or lessening the effects of frailty; prevention, amelioration or lessening the effects of at least one of an aging-related disease or conditions; increasing a health span or lifespan of the subject; increasing a stress resistance or resilience of the subject; increasing a rate or other enhancement of recovery after surgery, radiotherapy, disease and/or any other stress; prevention, amelioration or lesion the effects of menopausal syndrome; restoring reproductive function; elimination or lessening the spread of senescent cells; modulation of at least one biomarker of aging; a decrease in a rate of wrinkle development; and decrease in a rate of hair greying.

In these and other aspects, the anti-aging treatment is selected from the group consisting of a treatment related to changing a blood parameter, a heart rate, a cognitive function, a bone density, a basal metabolic rate, a systolic blood pressure, a heel bone mineral density (BMD), a heel quantitative ultrasound index (QUI), a heel broadband ultrasound attenuation, a forced expiratory volume in 1-second (FEV1), forced vital capacity (FVC), a peak expiratory flow (PEF), a duration to first press of snap-button in each round, a reaction time, a mean time to correctly identify matches, a right or left hand grip strength, a whole body fat-free mass, a leg fat-free mass, a time for recovery after a stress-inducing event, a resistance to radiation, a morbidity risk, and a mortality risk of the subject.

Also disclosed is a method of providing an anti-aging treatment or of treating or preventing an age-related disease or disorder of a subject. The method can comprise administering to the subject a pharmaceutical composition comprising an inhibitor of a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB; and at least one pharmaceutically acceptable excipient.

In addition, a method is also disclosed for providing an anti-aging treatment or of treating or preventing an age-related disease or disorder of a subject. The method comprises administering to the subject a pharmaceutical composition comprising a blood plasma fraction. The blood plasma fraction can comprise a negligible amount of a protein selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In some aspects, the negligible amount can be between about 0.008 pg/mL and about 15,000 ng/mL.

In other aspects, the negligible amount can be less than 15,000 ng/mL, less than 10,500 ng/mL, less than 7,350 ng/mL, less than 5,145 ng/mL, less than 3,602 ng/mL, less than 2,521 ng/mL, less than 1,765 ng/mL, less than 1,235 ng/mL, less than 865 ng/mL, less than 605 ng/mL, less than 424 ng/mL, less than 297 ng/mL, less than 208 ng/mL, less than 145 ng/mL, less than 102 ng/mL, less than 71 ng/mL, less than 50 ng/mL, less than 35 ng/mL, less than 24 ng/mL, less than 17 ng/mL, less than 12 ng/mL, less than 8 ng/mL, less than 6 ng/mL, less than 4 ng/mL, less than 3 ng/mL, less than 2 ng/mL, less than 1 ng/mL, less than 0.99 ng/mL, less than 0.69 ng/mL, less than 0.48 ng/mL, less than 0.34 ng/mL, less than 0.24 ng/mL, less than 0.17 ng/mL, less than 0.12 ng/mL, less than 0.08 ng/mL, less than 0.06 ng/mL, less than 0.04 ng/mL, less than 0.03 ng/mL, less than 0.02 ng/mL, less than 0.013 ng/mL, less than 0.009 ng/mL, less than 0.006 ng/mL, less than 0.004 ng/mL, less than 0.003 ng/mL, less than 0.002 ng/mL, less than 0.001 ng/mL, less than 0.0007 ng/mL, less than 0.0005 ng/mL, less than 0.0003 ng/mL, less than 0.0002 ng/mL, less than 0.0001 ng/mL, less than 0.08 pg/mL, less than 0.06 pg/mL, less than 0.04 pg/mL, less than 0.02 pg/mL, less than 0.01 pg/mL, or less than 0.008 pg/mL.

Also disclosed is a kit comprising a pharmaceutical composition and an instruction for using the pharmaceutical composition as part of the anti-aging treatment or to treat or prevent an age-related disease or disorder.

In some aspects, the pharmaceutical composition can comprise: an agent configured to bind to, inhibit, or degrade a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB and at least one pharmaceutically acceptable excipient.

In some aspects, the agent is selected from the group consisting of: a protein, a polymer, an aptamer, a SOMAmer, a peptide, a virus, a small molecule, a nanoparticle, an antibody, a monoclonal antibody, a polyclonal antibody, a humanized monoclonal antibody, a human monoclonal antibody, a human or humanized polyclonal antibody, an anti-GDF15 human antibody, an anti-FSTL3 human antibody, an anti-BMP4 human antibody, and an anti-FRZB (sFRP3) human antibody.

Also disclosed is a tangible medium comprising a computer program, which, when executed by one or more processors of a computing device, causes the computing device to attribute information regarding a protein binding device, another device, or a therapeutic agent with information about reducing, binding, inhibiting, or degrading at least one protein within the blood of the subject.

In some aspects, the protein can be selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB. In these and other aspects, the information about reducing, binding, inhibiting or degrading the protein can be associated with an anti-aging treatment.

In certain aspects, the tangible medium can be a computer storage medium.

### BRIEF DESCRIPTION OF THE DRAWINGS

The novel features of the invention are set forth with particularity in the appended claims and description. A better understanding of the features and advantages of the present invention will be obtained by reference to the following detailed description that sets forth illustrative embodiments, in which the principles of the invention are utilized, and the accompanying drawings of which:

FIG. 1 shows an example of a protein binding device described herein.

FIG. 2 shows one of many possible systems of treatment by protein binding device of this invention.

FIG. 3 shows results of mice experiment where some the Targets described herein were bound by the injected antibodies and reduction in mice biological age was measured. The more details on this test including its interpretation is given in Example 24.

FIG. 4 shows survival curves of certain animals in the treatment group versus the control group.

FIG. 5 shows the effects of the experimental treatment on the frailty of the treatment group versus the control group.

FIG. 6 shows the effects of the experimental treatment on the piloerection and coat condition of the treatment group versus the control group.

FIG. 7 shows the effects of the experimental treatment on Open Field test results.

FIG. 8 shows the effects of the experimental treatment on certain senescence markers.

FIG. 9 shows the effects of the experimental treatment on synaptoplasticity.

FIG. 10 shows protein expression in the whole trachea section semi-quantified under 40× microscope using an arbitrary score of 1-5.

FIG. 11 shows a sequence alignment of the amino acid sequence of the complete immunoglobulin heavy chain variable region of antibodies 01G06, 03G05, 04F08, 06C11, 08G01, 14F11, and 17B11.

FIG. 12 FIG. 12 shows a sequence alignment of the CDR1, CDR2, and CDR3 sequences for each of the immunoglobulin heavy chain variable region sequences in FIG. 11.

FIG. 13 shows a sequence alignment of the amino acid sequence of the complete immunoglobulin light chain variable region of antibodies 01G06, 03G05, 04F08,06C11,08G01,14F11, and 17B11.

FIG. 14 shows a sequence alignment of the CDR1, CDR2, and CDR3 sequences for each of the immunoglobulin light chain variable region sequences in FIG. 13.

FIG. 15A illustrates a fraction bound plot of the MST experiments HD22 aptamer binding to thrombin in buffer or serum.

FIG. 15B illustrates a fraction bound plot of the MST experiments HD1 aptamer binding to thrombin in buffer or serum

FIG. 16 illustrates one embodiment of a glass housing of an apheresis column.

### DETAILED DESCRIPTION

Disclosed herein are methods, devices, agents, pharmaceutical compositions, and systems for the binding and in some embodiments—selective binding of proteins in the blood and relative systems, methods and kits. In some embodiments, the elements other than proteins are modulated. In some embodiments, the protein targets are removed, inhibited or degraded by therapeutic means. In some embodiments, the removal of said proteins provides health benefits to the subject. In some embodiments, the removal of a target protein is effective in treating an age-related disorder, (including but not limited to Alzheimer's, Parkinson's, and Huntington's diseases, cardiovascular disease, renal failure, muscle wasting [cachexia], osteopenia or osteoporosis, obesity, insulin resistance or diabetes, and diverse adult-onset cancers etc.).

In some embodiments, the removal or inhibition or degradation of a target protein or modulation of its downstream or upstream effector is effective in decreasing the biological age of a patient or other anti-aging treatment.

### Definitions

The term “subject,” as used herein, generally refers to an animal, such as a mammalian species (e.g., mouse or human) or avian (i.e., bird) species, nematode (e.g., C. elegans), or other organism, such as a plant. More specifically, the subject can be a vertebrate, e.g., a mammal such as a mouse, a primate, a simian or a human. Preferably, the subject is a human. Preferably, the subject is more than 40 years old. Animals include, but are not limited to, farm animals, sport animals, and pets. A subject can be a healthy individual, an individual that has or is suspected of having a disease or a predisposition to the disease, or an individual that is in need of therapy or suspected of needing therapy, or an aged or frail individual. A subject can be any human being.

In some embodiments, by treating or preventing an age-related disease or disorder, any anti-aging treatment is meant. Anti-aging treatment includes (but is not limited to) treatments leading to prevention, amelioration or lessening the effects of aging, decreasing or delaying an increase in the biological age, slowing rate of aging; treatment, prevention, amelioration and lessening the effects of frailty or at least one of aging related diseases and conditions or declines or slowing down the progression of such decline (including but not limited to those indicated in Table 1, “Declines”), condition or disease, increasing health span or lifespan, rejuvenation, increasing stress resistance or resilience, increasing rate or other enhancement of recovery after surgery, radiotherapy, disease and/or any other stress, prevention and/or the treatment of menopausal syndrome, restoring reproductive function, eliminating or decrease in spreading of senescent cells, decreasing all-causes or multiple causes of mortality risks or mortality risks related to at least one or at least two of age related diseases or conditions or delaying in increase of such risks, decreasing morbidity risks. The treatment leading to the modulating at least one of biomarkers of aging into more youthful state or slowing down its change into “elder” state is also regarded to be an anti-aging treatment, including but not limited to biomarkers of aging which are visible signs of aging, such as wrinkles, grey hairs etc. In some embodiments, an age-related disease or disorder is selected from: atherosclerosis, cardiovascular disease, adult cancer, arthritis, cataracts, osteoporosis, type 2 diabetes, hypertension, neurodegeneration (including but not limited to Alzheimer's disease, Huntington's disease, and other age-progressive dementias; Parkinson's disease; and amyotrophic lateral sclerosis [ALS]), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence (including but not limited to age related decline in immune response to vaccines, age related decline in response to immunotherapy etc.), myocardial infarction, acute coronary syndrome, sarcopenia, sarcopenic obesity, senile osteoporosis, urinary incontinence etc. Aging-related changes in any parameter or physiological metric are also regarded as age-related conditions, including but not limited to aging related change in blood parameters, heart rate, cognitive functions/decline, bone density, basal metabolic rate, systolic blood pressure, heel bone mineral density (BMD), heel quantitative ultrasound index (QUI), heel broadband ultrasound attenuation, heel broadband ultrasound attenuation, forced expiratory volume in 1-second (FEV1), forced vital capacity (FVC), peak expiratory flow (PEF), duration to first press of snap-button in each round, reaction time, mean time to correctly identify matches, hand grip strength (right and/or left), whole body fat-free mass, leg fat-free mass (right and/or left), and time for recovery after any stress (wound, operation, chemotherapy, disease, change in lifestyle etc.). In some embodiments, the age-related disorder is a cardiovascular disease. In some embodiments, the age-related disorder is a bone loss disorder. In some embodiments, the age-related disorder is a neuromuscular disorder. In some embodiments, the age-related disorder is a neurodegenerative disorder or a cognitive disorder. In some embodiments, the age-related disorder is a metabolic disorder. In some embodiments, the age-related disorder is sarcopenia, osteoarthritis, chronic fatigue syndrome, senile dementia, mild cognitive impairment due to aging, schizophrenia, Huntington's disease, Pick's disease, Creutzfeldt-Jakob disease, stroke, CNS cerebral senility, age-related cognitive decline, pre-diabetes, diabetes, obesity, osteoporosis, coronary artery disease, cerebrovascular disease, heart attack, stroke, peripheral arterial disease, aortic valve disease, stroke, Lewy body disease, amyotrophic lateral sclerosis (ALS), mild cognitive impairment, pre-dementia, dementia, progressive subcortical gliosis, progressive supranuclear palsy, thalamic degeneration syndrome, hereditary aphasia, myoclonus epilepsy, macular degeneration, or cataracts. Aging related change in any parameter of organism is also regarded as an aging related condition, including but not limited to aging related change in at least one of the parameter selected from the Table “Declines”. In some embodiments, term “anti-aging treatment” means treatment of disease or condition mediated by Target. In some embodiments, term “anti-aging treatment” means treatment increasing resistance to radiation. In some embodiments, term “anti-aging treatment” means treatment of disease or condition associated with the alleviated level of protein selected from Targets in subject's fluid, in some embodiments such fluid is selected from whole blood, serum or plasma.

The term “binding element,” as used herein, generally refers to a molecule or any other object binding to Target. Optionally binding element could be linked to the stationary phase, e.g. sorbent or any other material and could enable Target extraction from the fluid, including but not limited to whole blood, serum or plasma. In some embodiments, the binding element is a capture agent, an object which captures Target directly or indirectly by any way.

In some embodiments of this invention, the fluid can be one or more selected of the following: a whole blood, blood fraction, blood with some blood components or elements deleted, blood with some added components or elements, serum, plasma. In a preferred embodiment, the fluid is plasma. In preferred embodiment the device of this invention reduces Targets from plasma.

In some embodiments of this invention “aged subject” is understood as a human being of chronological age (or in some embodiments, of biological age) of 30 years or older, 35 years or older, 40 years or older, 45 years or older, 50 years or older, 55 years or older, 60 years or older, 65 years or older, 70 years or older, 75 years or older, 80 years or older, 85 years or older, 90 years or older, 95 years or older. In some embodiments of this invention “aged subject” is understood as a frail human (or other animal).

In some embodiments, “attachment point” of the molecule refers to a atom or a chemical group which used for chemical modification which leads to molecule-linker conjugate. The obtained conjugate contains immobilization point in the linker.

In some embodiments, any one or any combination of the following proteins can further be referred as “Target” or “Targets”: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In some embodiments, effector upstream or downstream of a Target means element (protein, small molecule, cell, electrolytes, antibodies, antigens, hormones, microRNA, RNA etc.) which is upstream or downstream in a pathway in relation to Target. In some embodiments, effector upstream or downstream of a Target means any upstream or downstream element, which modulation or reduction mimics the effect of Target reduction, inhibition or degradation, optionally that have anti-aging effect.

In some embodiments, term “Target” effector upstream or downstream of a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB, optionally wherein modulation of such element produces an effect of anti-aging treatment. In some embodiments, everything related to at least one of the Target relates to a upstream or downstream of a such Target, wherein such upstream or downstream effect produces an effect of anti-aging treatment.

In some embodiments, term “Small molecule” means an individual compound with molecular weight less than about 2000 daltons in size, usually less than about 1500 daltons in size, more usually less than about 750 daltons in size, preferably less than about 500 daltons in size, although molecules larger than 2000 daltons in size will also be binding elements herein.

The terms “pharmaceutical composition” and formulation are used interchangeably herein.

The term particle is used for the objects administered by subjects, while term “stationary phase” is used for extracorporeal devices.”

The terms “selectively”/“selective binding” and “specifically”/“specifical binding” is/are used interchangeably herein.

The term “selected from . . . ” means “one or more of” (e.g. bind one or more of the following proteins . . . ”).

For example, the term “a protein selected from the group CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB” means “one or more of the proteins CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, or RGMB.” In this context, the term “binding element(s) selectively bind(s) a protein selected from the group CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB” can mean the following:

If one protein is to be bound, one binding element (e.g. an antibody, peptide, aptamer or the like) selectively binds one protein e.g. CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, or RGMB. If more than one protein is to be bound, a first binding element (e.g. an antibody, peptide, aptamer or the like) selectively binds a first protein (e.g. CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, or RGMB) and a second binding element selectively binds a second protein that is different to the first protein, and so on. For example, if more than one protein is to be bound, the first binding element can be an antibody specifically binding to a CCDC80 protein, and the second binding element can be an antibody specifically binding to a CD59 protein, and so on.

The term “platform” means a solid surface, such as a chip, while the stationary phase is meant to be a solid particulate or gel for use in an apheresis column or alike device.

When (an) amount(s) of binding element(s) is(are) given herein (e.g. 5 pmol, 10 pmol, 20 pmol, 50 pmol, 100 pmol, 0.3 μM, 0.5 μM, 1 μM, 5 μM, 10 μM or more, the given amount(s) refer(s) to the total amount of one binding element specifically binding to one protein per device.

The “pharmaceutical composition comprising a blood plasma fraction” as provided and defined herein is intended to encompass “modified plasma” or “modified blood” as described herein. The terms “pharmaceutical composition comprising a blood plasma fraction” and “modified plasma”/“modified blood”, respectively, can be used interchangeably herein.

### Protein Binding Devices

In certain aspects, the disclosure provides a device or method of use thereof for removing one or more proteins from plasma, wherein the proteins are selected from: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB. In certain embodiments, a device or method of use thereof removes or, in certain other embodiments—selectively removes two or more proteins, three or more proteins, four or more proteins, or five or more proteins selected from: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB. In certain embodiments, once the one or more proteins is removed from the plasma, the modified plasma may be reintroduced to a subject and thereby improve one or more diseases of aging, increase health span, rejuvenate, or reduce the biological age of the subject or in any other way be used as anti-aging treatment.

In certain embodiments, a protein binding device comprises a housing, wherein the housing defines a lumen and the housing further comprises an inlet port for receiving fluid to the lumen, and an outlet port for releasing fluid from the lumen, wherein the lumen comprises binding elements that selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB or in certain other embodiments wherein the lumen comprises different binding elements that selectively bind two or more proteins, three or more proteins, four or more proteins, or five or more proteins selected from: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

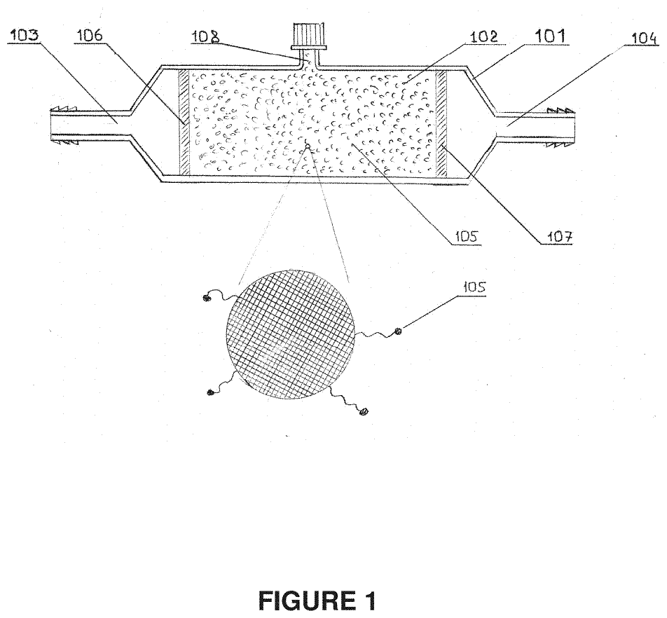

An exemplary device of the disclosure is depicted in FIG. 1. The housing is shown as 101, and which defines the lumen 102 on the interior of the housing. Inlet port 103 and outlet port 104 allow for the passage of plasma into the device through the inlet port and out of the device through the outlet port. The inlet or outlet port optionally comprises a mechanism for the attachment of tubing. In some embodiments, the attachments mechanism utilizes friction from grooves on the inlet port or outlet port, such as those seen in 103 or 104. The binding element connected to the stationary phase is depicted in 105, and is inside the lumen of the housing. The binding element and stationary phase is bordered by glass filters near the inlet port (106) and outlet port (107). The filters allow for the plasma to pass through the device, while keeping the stationary phase confined within the lumen. Optional port 108 allows for access to the stationary phase and binding element. Port 108 is useful in constructing the device and regenerating the device.

The housing of the device may comprise at least one inlet port, or at least two inlet ports. In some embodiments, the housing comprises at least one outlet port, or at least two outlet ports. In some embodiments, the housing comprises at least one inlet port and at least one outlet port. In some embodiments, the housing comprises an inlet port and an outlet port. In some embodiments, the housing comprises an inlet port, an outlet port, and an additional port of sufficient size to replace or regenerate the binding elements within the housing. In some embodiments, the housing defines a lumen and the housing further comprises an inlet port for receiving fluid to the lumen, and an outlet port for releasing fluid from the lumen. In some embodiments, the housing is comprised of at least one plastic material. In some embodiments, the housing is comprised of at least one metal material. In some embodiments, the housing is comprised of at least one rubber or rubber like material. In some embodiments, the housing is comprised of at least one flexible material. In some embodiments, the housing is comprised of at least one non-flexible material. In some embodiments, the housing is a plastic column. In some embodiments, the housing is a metal column. In some embodiments, the housing is a flexible bag, e.g., a plastic or polymeric bag.

In certain embodiments, the lumen of the housing has a fluid capacity of 5 mL or greater. In certain embodiments, the lumen of the housing has a fluid capacity of 10 mL or greater. In certain embodiments, the lumen of the housing has a fluid capacity of 15 mL or greater. In certain embodiments, the lumen of the housing has a fluid capacity of 20 mL or greater. In certain embodiments, the lumen of the housing has a fluid capacity of 25 mL or greater. In certain embodiments, the lumen of the housing has a fluid capacity of 50 mL or greater. In certain embodiments, the lumen of the housing has a fluid capacity of 75 mL or greater. In certain embodiments, the lumen of the housing has a fluid capacity of 100 mL or greater. In certain embodiments, the lumen of the housing has a fluid capacity of 200 mL or greater. In certain embodiments, the lumen of the housing has a fluid capacity of about 5 mL to about 500 mL greater. In certain embodiments, the lumen of the housing has a fluid capacity of about 10 mL to about 300 mL greater. In certain embodiments, the lumen of the housing has a fluid capacity of about 15 mL to about 200 mL greater. In certain embodiments, the lumen of the housing has a fluid capacity of about 20 mL to about 175 mL greater. In certain embodiments, the lumen of the housing has a fluid capacity of about 25 mL to about 150 mL greater. In certain embodiments, the lumen of the housing has a fluid capacity of about 30 mL to about 125 mL greater. In certain embodiments, the lumen of the housing has a fluid capacity of about 50 mL to about 100 mL greater. The fluid capacity of the housing is the maximum volume of fluid that the housing of the device can contain when the outlet port is closed. The fluid capacity of the housing may be diminished in a housing with a stationary phase relative to a housing without a stationary phase.

In some embodiments, the protein binding device of this disclosure allows purification of plasma from the at least one of the Targets, while at least 10%, 25%, 50%, 75%, 85%, 90%, 95% of at least 3000 proteins of output blood plasma or the components from the table “Biomarkers” are preserved on input level or median difference in percentage in concentrations of such proteins or components from the input level is significantly less than the difference in percentage in concentrations between input and output level of the reduced Target or the share of Target mass in the mass of the extracted proteins is at least 2, 4, 10, 25, 60, 140, 330, 770, 1820, 4270, 10000 times more than the share of the mass of same Target in the overall protein mass in input plasma (plasma or other fluid entering the device of this invention).

In some embodiments, a binding element is used to capture a Target protein by contacting and binding the Target protein. In some embodiments, the binding element selectively binds a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB. In some embodiments, a binding element is considered to be selective for binding Target if it binds human Target with greater affinity than it binds one or more of several proteins considered human homologs of Target or with greater affinity than it binds one or more other proteins. In some embodiments, the binding element selectively binds CCDC80. In some embodiments, the binding element selectively binds CD59. In some embodiments, the binding element selectively binds CHRDL1. In some embodiments, the binding element selectively binds COL18A1. In some embodiments, the binding element selectively binds CST3. In some embodiments, the binding element selectively binds DPT. In some embodiments, the binding element selectively binds EFEMP1. In some embodiments, the binding element selectively binds FAS. In some embodiments, the binding element selectively binds FSTL3. In some embodiments, the binding element selectively binds GAS1. In some embodiments, the binding element selectively binds GDF15. In some embodiments, the binding element selectively binds KLK11. In some embodiments, the binding element selectively binds MMP7. In some embodiments, the binding element selectively binds NBL1. In some embodiments, the binding element selectively binds NTN1. In some embodiments, the binding element selectively binds POSTN. In some embodiments, the binding element selectively binds PTN. In some embodiments, the binding element selectively binds RELT. In some embodiments, the binding element selectively binds SFRP1. In some embodiments, the binding element selectively binds SMOC1. In some embodiments, the binding element selectively binds STC1. In some embodiments, the binding element selectively binds TNFRSF1A. In some embodiments, the binding element selectively binds UNC5C. In some embodiments, the binding element selectively binds sFRP-3. In some embodiments, the binding element selectively binds TNFRSF1B. In some embodiments, the binding element selectively binds CD55. In some embodiments, the binding element selectively binds BMP4. In some embodiments, the binding element selectively binds RGMB. In some embodiments, the binding elements selectively bind a protein selected from GDF15, FSTL3, FRZB (sFRP3), and BMP4.

The Targets were selected based on proteomic data analysis of blood samples from longitudinal study with the help of mathematical model described in Podolskiy, D. et al. Critical dynamics of gene networks is a mechanism behind ageing and gompertz law. arXiv preprint arXiv:1502.04307v2 [q-bio.MN] (2015) and one Target—BMP4 based on the analysis of pathways the other Targets belong to. A selection of proteins are described herein, along with full names and corresponding UnitProt numbers, in the following list: CCDC80 (Coiled-coil domain-containing protein 80, Q76M96), CD59 (CD59 glycoprotein, P13987), CHRDL1 (Chordin-like protein 1, Q9BU40), COL18A1 (Endostatin, P39060), CST3 (Cystatin-C, P01034), DPT (Dermatopontin, Q07507), EFEMP1 (EGF-containing fibulin-like extracellular matrix protein 1, Q12805), FAS (Tumor necrosis factor receptor superfamily member 6, P25445), FSTL3 (Follistatin-related protein 3, 095633), GAS1 (Growth arrest-specific protein 1, P54826), GDF15 (Growth/differentiation factor 15, Q99988), KLK11(Kallikrein-11, Q9UBX7), MMP7 (Matrilysin, P09237), NBL1 (Neuroblastoma suppressor of tumorigenicity 1, P41271), NTN1 (Netrin-1, 095631), POSTN (Periostin, Q15063), PTN (Pleiotrophin, P21246), RELT (Tumor necrosis factor receptor superfamily member 19L, Q969Z4), SFRP1 (Secreted frizzled-related protein 1, Q8N474), SMOC1 (SPARC-related modular calcium-binding protein 1, Q9H4F8), STC1 (Stanniocalcin-1, P52823), TNFRSF1A (Tumor necrosis factor receptor superfamily member 1A, P19438), UNC5C (Netrin receptor UNC5C, 095185), sFRP-3 (FRZB) (Secreted frizzled-related protein 3, Q92765), TNFRSF1B (Uniprot P20333), CD55 (Uniprot P08174), RGMB (Uniprot Q6NW40); UNC5C (UniProtKB—O95185), sFRP-3 (UniProtKB—Q92765), TNFRSF1B (UniProtKB—P20333); BMP4 (UniProtKB—P12644); and B2M (Beta-2-microglobulin, and UniProt number: P61769). In some embodiments, the proteins described herein are denaturated or abnormally folded.

In some embodiments, the proteins described herein comprise the following sequences:

The target “Tumor necrosis factor receptor superfamily member 1A” has the sequence:
[SEQ ID NO: 1] MGLSTVPDLLLPLVLLELLVGIYPSGVIGLVPHLGDREKRDSVCPQGKYIHPQNNSICCT KCHKGTYLYNDCPGPGQDTDCRECESGSFTASENHLRHCLSCSKCRKEMGQVEISS CTVDRDTVCGCRKNQYRHYWSENLFQCFNCSLCLNGTVHLSCQEKQNTVCTCHAGF FLRENECVSCSNCKKSLECTKLCLPQIENVKGTEDSGTTVLLPLVIFFGLCLLSLLFIGLM YRYQRWKSKLYSIVCGKSTPEKEGELEGTTTKPLAPNPSFSPTPGFTPTLGFSPVPSS TFTSSSTYTPGDCPNFAAPRREVAPPYQGADPILATALASDPIPNPLQKWEDSAHKPQ SLDTDDPATLYAVVENVPPLRWKEFVRRLGLSDHEIDRLELQNGRCLREAQYSMLAT WRRRTPRREATLELLGRVLRDMDLLGCLEDIEEALCGPAALPPAPSLLR; this sequence can have the following modifications/variations. Glycosylation: amino acids 54-54 {N-linked (GlcNAc . . . ) asparagine}; amino acids 145-145 {N-linked (GlcNAc . . . ) asparagine}; amino acids 151-151 {N-linked (GlcNAc . . . ) asparagine}; Disulfide bond: amino acids 44-58; amino acids 59-72; amino acids 62-81; amino acids 84-99; amino acids 102-117; amino acids 105-125; amino acids 127-143; amino acids 146-158; amino acids 149-166; amino acids 168-179; amino acids 182-195; amino acids 185-191; Sequence conflict: amino acids 13-13 {L->LILPQ}; amino acids 255-255 {K->E}; amino acids 286-286 {S->G}; amino acids 394-394 {R->L}; amino acids 412-412 {Missing}; amino acids 443-446 {GPAA->APP}; Natural variant: amino acids 51-51 {in FHF. H->Q}; amino acids 59-59 {in FHF. C->R}; amino acids 59-59 {in FHF. C->S}; amino acids 62-62 {in FHF. C->G}; amino acids 62-62 {in FHF. C->Y}; amino acids 75-75 {in FHF %3B may be a polymorphism. P->L}; amino acids 79-79 {in FHF. T->M}; amino acids 81-81 {in FHF. C->F}; amino acids 99-99 {in FHF. C->S}; amino acids 115-115 {in FHF. S->G}; amino acids 117-117 {in FHF. C->R}; amino acids 117-117 {in FHF. C->Y}; amino acids 121-121 {in FHF. R->P}; amino acids 121-121 {in FHF %3B unknown pathological significance. R->Q}; amino acids 305-305 {P->T}; Alternative sequence: amino acids 1-232 {in isoform 3. Missing}; amino acids 1-108 {in isoform 2. Missing}; amino acids 184-455 {in isoform 4. NCKKSLECTKLCLPQIENVKGTEDSGTTVLLPLVIFFGLCLLSLLFIGLMYRYQRWKSKL YSIVCGKSTPEKEGELEGTTTKPLAPNPSFSPTPGFTPTLGFSPVPSSTFTSSSTYTPG DCPNFAAPRREVAPPYQGADPILATALASDPIPNPLQKWEDSAHKPQSLDTDDPATLY AVVENVPPLRWKEFVRRLGLSDHEIDRLELQNGRCLREAQYSMLATWRRRTPRREAT LELLGRVLRDMDLLGCLEDIEEALCGPAALPPAPSLLR->KHHSAVAPGHFLWSLPFIPPLHWFNVSLPTVEVQALLHCLWEIDT}; amino acids 184-218 {in isoform 5. NCKKSLECTKLCLPQIENVKGTEDSGTTVLLPLVI->KVLLCRPGWNAVARSRLTATSASQIQAILLLQPPK}; amino acids 219-455 {in isoform 5. Missing}; Natural variant: amino acids 51-51 {in FHF. H->Q}; amino acids 59-59 {in FHF. C->R}; amino acids 59-59 {in FHF. C->S}; amino acids 62-62 {in FHF. C->G}; amino acids 62-62 {in FHF. C->Y}; amino acids 75-75 {in FHF %3B may be a polymorphism. P->L}; amino acids 79-79 {in FHF. T->M}; amino acids 81-81 {in FHF. C->F}; amino acids 99-99 {in FHF. C->S}; amino acids 115-115 {in FHF. S->G}; amino acids 117-117 {in FHF. C->R}; amino acids 117-117 {in FHF. C->Y}; amino acids 121-121 {in FHF. R->P}; amino acids 121-121 {in FHF %3B unknown pathological significance. R->Q}; amino acids 305-305 {P->T}; The target “Growth arrest-specific protein 1” has the sequence: [SEQ ID NO: 2] MVAALLGGGGEARGGTVPGAWLCLMALLQLLGSAPRGSGLAHGRRLICWQALLQCQ GEPECSYAYNQYAEACAPVLAQHGGGDAPGAAAAAFPASAASFSSRWRCPSHCISAL IQLNHTRRGPALEDCDCAQDENCKSTKRAIEPCLPRTSGGGAGGPGAGGVMGCTEA RRRCDRDSRCNLALSRYLTYCGKVFNGLRCTDECRTVIEDMLAMPKAALLNDCVCDG LERPICESVKENMARLCFGAELGNGPGSSGSDGGLDDYYDEDYDDEQRTGGAGGEQ PLDDDDGVPHPPRPGSGAAASGGRGDLPYGPGRRSSGGGGRLAPRGAWTPLASILL LLLGPLF; this sequence can have the following modifications/variations. Lipidation: amino acids 318-318 {GPI-anchor amidated serine}; Glycosylation: amino acids 117-117 {N-linked (GlcNAc . . . ) asparagine}; Sequence conflict: amino acids 216-216 {A->V}; The target “SPARC-related modular calcium-binding protein 1” has the sequence: [SEQ ID NO: 3] MLPARCARLLTPHLLLVLVQLSPARGHRTTGPRFLISDRDPQCNLHCSRTQPKPICASD G RSYESMCEYQRAKCRDPTLGVVHRGRCKDAGQSKCRLERAQALEQAKKPQEAVFVP ECGEDGSFTQVQCHTYTGYCWCVTPDGKPISGSSVQNKTPVCSGSVTDKPLSQGNS GRKDDGSKPTPTMETQPVFDGDEITAPTLWIKHLVIKDSKLNNTNIRNSEKVYSCDQER QSALEEAQQNPREGIVIPECAPGGLYKPVQCHQSTGYCWCVLVDTGRPLPGTSTRYV MPSCESDARAKTTEADDPFKDRELPGCPEGKKMEFITSLLDALTTDMVQAINSAAPTG GGRFSEPDPSHTLEERVVHWYFSQLDSNSSNDINKREMKPFKRYVKKKAKPKKCARR FTDYCDLNKDKVISLPE LKGCLGVSKEGRLV; this sequence can have the following modifications/variations. Glycosylation: amino acids 214-214 {N-linked (GlcNAc . . . ) asparagine}; amino acids 374-374 {N-linked (GlcNAc . . . ) asparagine}; Disulfide bond: amino acids 43-74; amino acids 47-67; amino acids 56-87; amino acids 95-118; amino acids 129-136; amino acids 138-158; amino acids 227-251; amino acids 262-269; amino acids 271-292; Natural variant: amino acids 82-82 {V->M}; amino acids 278-278 {in OAS. R->C}; amino acids 283-283 {in OAS. T->N}; amino acids 286-286 {in OAS. R->H}; Alternative sequence: amino acids 430-430 {in isoform 2. E->EV}; Natural variant: amino acids 82-82 {V->M}; amino acids 278-278 {in OAS. R->C}; amino acids 283-283 {in OAS. T->N}; amino acids 286-286 {in OAS. R->H}; The target “Matrilysin” has the sequence: [SEQ ID NO: 4] MRLTVLCAVCLLPGSLALPLPQEAGGMSELQWEQAQDYLKRFYLYDSETKNANSLEA KLK EMQKFFGLPITGMLNSRVIEIMQKPRCGVPDVAEYSLFPNSPKWTSKVVTYRIVSYTRD L PHITVDRLVSKALNMWGKEIPLHFRKVVWGTADIMIGFARGAHGDSYPFDGPGNTLAH AF APGTGLGGDAHFDEDERWTDGSSLGINFLYAATHELGHSLGMGHSSDPNAVMYPTY GNGD PQNFKLSQDDIKGIQKLYGKRSNSRKK; this sequence can have the following modifications/variations. Natural variant: amino acids 77-77 {R->H}; amino acids 137-137 {G->D}; amino acids 241-241 {P->L}; Natural variant: amino acids 77-77 {R->H}; amino acids 137-137 {G->D}; amino acids 241-241 {P->L}; The target “Netrin receptor UNC5C” has the sequence: [SEQ ID NO: 5] MRKGLRATAARCGLGLGYLLQMLVLPALALLSASGTGSAAQDDDFFHELPETFPSDPP EP LPHFLIEPEEAYIVKNKPVNLYCKASPATQIYFKCNSEWVHQKDHIVDERVDETSGLIVR EVSIEISRQQVEELFGPEDYWCQCVAWSSAGTTKSRKAYVRIAYLRKTFEQEPLGKEV SL EQEVLLQCRPPEGIPVAEVEWLKNEDIIDPVEDRNFYITIDHNLIIKQARLSDTANYTCV AKNIVAKRKSTTATVIVYVNGGWSTWTEWSVCNSRCGRGYQKRTRTCTNPAPLNGG AFCEGQSVQKIACTTLCPVDGRWTPWSKWSTCGTECTHWRRRECTAPAPKNGGKD CDGLVLQSKNCTDGLCMQTAPDSDDVALYVGIVIAVIVCLAISVVVALFVYRKNHRDFE SDIIDSSALN GGFQPVNIKAARQDLLAVPPDLTSAAAMYRGPVYALHDVSDKIPMTNSPILDPLPNLKI K VYNTSGAVTPQDDLSEFTSKLSPQMTQSLLENEALSLKNQSLARQTDPSCTAFGSFNS LG GHLIVPNSGVSLLIPAGAIPQGRVYEMYVTVHRKETMRPPMDDSQTLLTPVVSCGPPG AL LTRPVVLTMHHCADPNTEDWKILLKNQAAQGQWEDVVVVGEENFTTPCYIQLDAEAC HIL TENLSTYALVGHSTTKAAAKRLKLAIFGPLCCSSLEYSIRVYCLDDTQDALKEILHLERQ MGGQLLEEPKALHFKGSTHNLRLSIHDIAHSLWKSKLLAKYQEIPFYHVWSGSQRNLH CT FTLERFSLNTVELVCKLCVRQVEGEGQIFQLNCTVSEEPTGIDLPLLDPANTITTVTGPS AFSIPLPIRQKLCSSLDAPQTRGHDWRMLAHKLNLDRYLNYFATKSSPTGVILDLWEAQ NFPDGNLSMLAAVLEEMGRHETVVSLAAEGQY; this sequence can have the following modifications/variations. Glycosylation: amino acids 236-236 {N-linked (GlcNAc . . . ) asparagine}; amino acids 361-361 {N-linked (GlcNAc . . . ) asparagine}; Disulfide bond: amino acids 83-144; amino acids 95-142; amino acids 188-239; amino acids 272-309; amino acids 276-313; amino acids 287-299; amino acids 328-362; amino acids 332-367; amino acids 340-352; Sequence conflict: amino acids 219-219 {T->I}; amino acids 489-489 {T->S}; amino acids 651-651 {Q->K}; Natural variant: amino acids 37-37 {G->V}; amino acids 721-721 {M->T}; amino acids 841-841 {A->T}; Alternative sequence: amino acids 370-370 {in isoform 2. T->SFIYPISTEQRTQNEYGFSS}; amino acids 579-931 {in isoform 2. Missing}; Modified residue: amino acids 502-502 {Phosphoserine}; amino acids 568-568 {Phosphotyrosine}; Natural variant: amino acids 37-37 {G->V}; amino acids 721-721 {M->T}; amino acids 841-841 {A->T}; The target “Dermatopontin” has the sequence: [SEQ ID NO: 6] MDLSLLWVLLPLVTMAWGQYGDYGYPYQQYHDYSDDGWVNLNRQGFSYQCPQGQV IVAVRSIFSKKEGSDRQWNYACMPTPQSLGEPTECWWEEINRAGMEWYQTCSNNGL VAGFQSRYFESVLDREWQFYCCRYSKRCPYSCWLTTEYPGHYGEEMDMISYNYDYYI RGATTTFSAVERDRQWKFIMCRMTEYDCEFANV; this sequence can have the following modifications/variations. Disulfide bond: amino acids 50-77; amino acids 90-132 {Or C-90 with C-133}; amino acids 106-133 {Or C-106 with C-132}; amino acids 139-196; amino acids 143-189; Sequence conflict: amino acids 10-10 {L->M}; amino acids 147-147 {T->I}; Natural variant: amino acids 201-201 {V->I}; Modified residue: amino acids 19-19 {Pyrrolidone carboxylic acid}; amino acids 23-23 {Sulfotyrosine}; amino acids 162-162 {Sulfotyrosine}; amino acids 164-164 {Sulfotyrosine}; amino acids 166-166 {Sulfotyrosine}; amino acids 167-167 {Sulfotyrosine}; amino acids 194-194 {Sulfotyrosine}; Natural variant: amino acids 201-201 {V->I}; The target “EGF-containing fibulin-like extracellular matrix protein 1” has the sequence: [SEQ ID NO: 7] MLKALFLTMLTLALVKSQDTEETITYTQCTDGYEWDPVRQQCKDIDECDIVPDACKGG MK CVNHYGGYLCLPKTAQIIVNNEQPQQETQPAEGTSGATTGVVAASSMATSGVLPGGG FVASAAAVAGPEMQTGRNNFVIRRNPADPQRIPSNPSHRIQCAAGYEQSEHNVCQDID ECTAGTHNCRADQVCINLRGSFACQCPPGYQKRGEQCVDIDECTIPPYCHQRCVNTP GSFYCQCSPGFQLAANNYTCVDINECDASNQCAQQCYNILGSFICQCNQGYELSSDR LNCEDIDECRTSSYLCQYQCVNEPGKFSCMCPQGYQVVRSRTCQDINECETTNECRE DEMCWNYHGGFRCYPRNPCQDPYILTPENRCVCPVSNAMCRELPQSIVYKYMSIRSD RSVPSDIFQIQATTIYANTINTFRIKSGNENGEFYLRQTSPVSAMLVLVKSLSGPREHIVD LEMLTVSSIGTFRTSSVLRLTIIVGPFSF; this sequence can have the following modifications/variations. Glycosylation: amino acids 249-249 {N-linked (GlcNAc . . . ) asparagine}; Disulfide bond: amino acids 177-190; amino acids 184-199; amino acids 201-212; amino acids 218-228; amino acids 224-237; amino acids 239-252; amino acids 258-268; amino acids 264-277; amino acids 279-292; amino acids 298-309; amino acids 305-318; amino acids 320-332; amino acids 338-350; amino acids 344-359; amino acids 365-377; Natural variant: amino acids 220-220 {I->F}; amino acids 345-345 {in DHRD %3B misfolded %2C accumulates in cells due to inefficient secretion %3B induces the formation of deposits between Bruch's membrane and the retinal pigment epithelium where it accumulates. R->W}; Alternative sequence: amino acids 1-58 {in isoform 5. Missing}; amino acids 1-8 {in isoform 2. Missing}; amino acids 58-58 {in isoform 3. Missing}; amino acids 106-106 {in isoform 4. Missing}; amino acids 214-293 {in isoform 5. Missing}; Natural variant: amino acids 220-220 {I->F}; amino acids 345-345 {in DHRD %3B misfolded %2C accumulates in cells due to inefficient secretion %3B induces the formation of deposits between Bruch's membrane and the retinal pigment epithelium where it accumulates. R->W};

The target “Stanniocalcin-1” has the sequence:

[SEQ ID NO: 8] MLQNSAVLLVLVISASATHEAEQNDSVSPRKSRVAAQNSAEVVRCLNSALQVGCGAFA CLENSTCDTDGMYDICKSFLYSAAKFDTQGKAFVKESLKCIANGVTSKVFLAIRRCSTF QRMIAEVQEECYSKLNVCSIAKRNPEAITEVVQLPNHFSNRYYNRLVRSLLECDEDTVS TIRDSLMEKIGPNMASLFHILQTDHCAQTHPRADFNRRRTNEPQKLKVLLRNLRGEED SPSHIK RTSHESA; this sequence can have the following modifications/variations. Glycosylation: amino acids 62-62 {N-linked (GlcNAc . . . ) asparagine}; Disulfide bond: amino acids 45-59; amino acids 54-74; amino acids 65-114; amino acids 98-128; amino acids 135-170; amino acids 202-202 {Interchain}; Alternative sequence: amino acids 1-69 {in isoform 2. Missing};
The target “Follistatin-related protein 3” has the sequence:
[SEQ ID NO: 9] MRPGAPGPLWPLPWGALAWAVGFVSSMGSGNPAPGGVCWLQQGQEATCSLVLQT DVTRAECCASGNIDTAWSNLTHPGNKINLLGFLGLVHCLPCKDSCDGVECGPGKACR MLGGRPRCECAPDCSGLPARLQVCGSDGATYRDECELRAARCRGHPDLSVMYRGR CRKSCEHVVCPRPQSCVVDQTGSAHCVVCRAAPCPVPSSPGQELCGNNNVTYISSC HMRQATCFLGRSIGVRHAGSCAGTPEEPPGGESAEEEENFV; this sequence can have the following modifications/variations. Glycosylation: amino acids 73-73 {N-linked (GlcNAc . . . ) asparagine}; amino acids 215-215 {N-linked (GlcNAc . . . ) asparagine}; Disulfide bond: amino acids 38-61; amino acids 48-92; amino acids 62-95; amino acids 99-110; amino acids 104-119; amino acids 121-153; amino acids 125-146; amino acids 135-167; amino acids 171-182; amino acids 176-192; amino acids 195-229; amino acids 200-222; amino acids 211-243; Alternative sequence: amino acids 1-26 {in isoform 2. Missing}; Modified residue: amino acids 255-255 {Phosphoserine %3B by FAM20C}; Mutagenesis: amino acids 27-27 {Nuclear localization. M->A};
The target “Tumor necrosis factor receptor superfamily member 6” has the sequence:
[SEQ ID NO: 10] MLGIWTLLPLVLTSVARLSSKSVNAQVTDINSKGLELRKTVTTVETQNLEGLHHDGQFC HKPCPPGERKARDCTVNGDEPDCVPCQEGKEYTDKAHFSSKCRRCRLCDEGHGLEV EINCTRTQNTKCRCKPNFFCNSTVCEHCDPCTKCEHGIIKECTLTSNTKCKEEGSRSNL GWLCLLLLPIPLIVWVKRKEVQKTCRKHRKENQGSHESPTLNPETVAINLSDVDLSKYIT TIAGVMTLSQVKGFVRKNGVNEAKIDEIKNDNVQDTAEQKVQLLRNWHQLHGKKEAY DTLIKDLKKANLCTLAEKIQTIILKDITSDSENSNFRNEIQSLV; this sequence can have the following modifications/variations. Glycosylation: amino acids 28-28 {O-linked (GalNAc . . . ) threonine}; amino acids 118-118 {N-linked (GlcNAc . . . ) asparagine}; amino acids 136-136 {N-linked (GlcNAc . . . ) asparagine}; Disulfide bond: amino acids 59-73; amino acids 63-82; amino acids 85-101; amino acids 104-119; amino acids 107-127; amino acids 129-143; amino acids 146-157; amino acids 149-165; Sequence conflict: amino acids 224-224 {L->F}; amino acids 242-242 {L->P}; Natural variant: amino acids 16-16 {A->T}; amino acids 25-25 {in non-Hodgkin lymphoma %3B somatic mutation. A->T}; amino acids 28-28 {in ALPS1A %3B associated with autoimmune hepatitis type 2. T->A}; amino acids 82-82 {in ALPS1A. C->R}; amino acids 118-118 {in squamous cell carcinoma %3B burn-scar related %3B somatic mutation. N->S}; amino acids 121-121 {in ALPS1A. R->W}; amino acids 122-122 {T->I}; amino acids 178-178 {in squamous cell carcinoma %3B burn-scar related %3B somatic mutation. C->R}; amino acids 180-180 {in non-Hodgkin lymphoma %3B somatic mutation. L->F}; amino acids 183-183 {in non-Hodgkin lymphoma %3B somatic mutation. P->L}; amino acids 184-184 {I->V}; amino acids 198-198 {in non-Hodgkin lymphoma %3B somatic mutation. T->I}; amino acids 232-232 {in ALPS1A %3B no effect on interaction with FADD. Y->C}; amino acids 241-241 {in ALPS1A. T->K}; amino acids 241-241 {in ALPS1A. T->P}; amino acids 249-249 {in ALPS1A. V->L}; amino acids 250-250 {in ALPS1A. R->P}; amino acids 250-250 {in ALPS1A %3B no effect on interaction with FADD. R->Q}; amino acids 253-253 {in ALPS1A. G->D}; amino acids 253-253 {in ALPS1A. G->S}; amino acids 255-255 {in squamous cell carcinoma %3B burn-scar related %3B somatic mutation. N->D}; amino acids 257-257 {in ALPS1A %3B loss of interaction with FADD. A->D}; amino acids 259-259 {in ALPS1A. I->R}; amino acids 260-260 {in ALPS1A. D->G}; amino acids 260-260 {in ALPS1A %3B also found in non-Hodgkin lymphoma %3B somatic mutation %3B loss of interaction with FADD. D->V}; amino acids 260-260 {in ALPS1A %3B loss of interaction with FADD. D->Y}; amino acids 262-262 {in ALPS1A. I->S}; amino acids 264-264 {in non-Hodgkin lymphoma %3B somatic mutation. N->K}; amino acids 270-270 {in ALPS1A. T->1}; amino acids 270-270 {in ALPS1A %3B loss of interaction with FADD. T->K}; amino acids 272-272 {in ALPS1A. E->G}; amino acids 272-272 {in ALPS1A %3B also found in non-Hodgkin lymphoma %3B somatic mutation %3B loss of interaction with FADD. E->K}; amino acids 278-278 {in non-Hodgkin lymphoma %3B somatic mutation. L->F}; amino acids 299-299 {in non-Hodgkin lymphoma %3B somatic mutation. K->N}; amino acids 305-305 {T->I}; amino acids 310-310 {in ALPS1A. I->S}; Alternative sequence: amino acids 66-103 {in isoform 2. GERKARDCTVNGDEPDCVPCQEGKEYTDKAHFSSKCRR->DVNMESSRNAHSPATPSAKRKDPDLTWGGFVFFFCQFH}; amino acids 66-86 {in isoform 3. GERKARDCTVNGDEPDCVPCQ->DVNMESSRNAHSPATPSAKRK}; amino acids 87-335 {in isoform 3. Missing}; amino acids 104-335 {in isoform 2. Missing}; amino acids 112-149 {ln isoform 4. GLEVEINCTRTQNTKCRCKPNFFCNSTVCEHCDPCTKC->DVNMESSRNAHSPATPSAKRKDPDLTWGGFVFFFCQFH}; amino acids 112-132 {in isoform 5. GLEVEINCTRTQNTKCRCKPN->DVNMESSRNAHSPATPSAKRK}; amino acids 133-335 {in isoform 5. Missing}; amino acids 150-335 {in isoform 4. Missing}; amino acids 169-189 {in isoform 6. Missing}; amino acids 218-220 {in isoform 7. ETV->MLT}; amino acids 221-335 {in isoform 7. Missing}; Modified residue: amino acids 209-209 {Phosphoserine}; amino acids 214-214 {Phosphothreonine}; amino acids 225-225 {Phosphoserine}; Mutagenesis: amino acids 250-250 {Strongly decreased interaction with FADD. R->E}; amino acids 261-261 {Loss of interaction with FADD. E->K}; amino acids 283-283 {Loss of interaction with FADD. Q->K}; amino acids 287-287 {Strongly decreased interaction with FADD. K->D}; amino acids 291-291 {Decreased interaction with FADD. Y->D}; amino acids 313-313 {Constitutive activation. Promotes apoptosis %2C both in the presence and in the absence of stimulation by a ligand. I->D}; Natural variant: amino acids 16-16 {A->T}; amino acids 25-25 {in non-Hodgkin lymphoma %3B somatic mutation. A->T}; amino acids 28-28 {in ALPS1A %3B associated with autoimmune hepatitis type 2. T->A}; amino acids 82-82 {in ALPS1A. C->R}; amino acids 118-118 {ln squamous cell carcinoma %3B burn-scar related %3B somatic mutation. N->S}; amino acids 121-121 {in ALPS1A. R->W}; amino acids 122-122 {T->I}; amino acids 178-178 {ln squamous cell carcinoma %3B burn-scar related %3B somatic mutation. C->R}; amino acids 180-180 {in non-Hodgkin lymphoma %3B somatic mutation. L->F}; amino acids 183-183 {in non-Hodgkin lymphoma %3B somatic mutation. P->L}; amino acids 184-184 {I->V}; amino acids 198-198 {in non-Hodgkin lymphoma %3B somatic mutation. T->I}; amino acids 232-232 {in ALPS1A %3B no effect on interaction with FADD. Y->C}; amino acids 241-241 {in ALPS1A. T->K}; amino acids 241-241 {in ALPS1A. T->P}; amino acids 249-249 {in ALPS1A. V->L}; amino acids 250-250 {in ALPS1A. R->P}; amino acids 250-250 {ln ALPS1A %3B no effect on interaction with FADD. R->Q}; amino acids 253-253 {in ALPS1A. G->D}; amino acids 253-253 {in ALPS1A. G->S}; amino acids 255-255 {in squamous cell carcinoma %3B burn-scar related %3B somatic mutation. N->D}; amino acids 257-257 {in ALPS1A %3B loss of interaction with FADD. A->D}; amino acids 259-259 {in ALPS1A. I->R}; amino acids 260-260 {in ALPS1A. D->G}; amino acids 260-260 {in ALPS1A %3B also found in non-Hodgkin lymphoma %3B somatic mutation %3B loss of interaction with FADD. D->V}; amino acids 260-260 {in ALPS1A %3B loss of interaction with FADD. D->Y}; amino acids 262-262 {in ALPS1A. I->S}; amino acids 264-264 {in non-Hodgkin lymphoma %3B somatic mutation. N->K}; amino acids 270-270 {in ALPS1A. T->I}; amino acids 270-270 {in ALPS1A %3B loss of interaction with FADD. T->K}; amino acids 272-272 {in ALPS1A. E->G}; amino acids 272-272 {in ALPS1A %3B also found in non-Hodgkin lymphoma %3B somatic mutation %3B loss of interaction with FADD. E->K}; amino acids 278-278 {in non-Hodgkin lymphoma %3B somatic mutation. L->F}; amino acids 299-299 {in non-Hodgkin lymphoma %3B somatic mutation. K->N}; amino acids 305-305 {T->I}; amino acids 310-310 {in ALPS1A. I->S};
The target “Netrin-1” has the sequence:
[SEQ ID NO: 11] MMRAVWEALAALAAVACLVGAVRGGPGLSMFAGQAAQPDPCSDENGHPRRCIPDFV NAAFGKDVRVSSTCGRPPARYCVVSERGEERLRSCHLCNASDPKKAHPPAFLTDLNN PHNLTCWQSENYLQFPHNVTLTLSLGKKFEVTYVSLQFCSPRPESMAIYKSMDYGRT WVPFQFYSTQCRKMYNRPHRAPITKQNEQEAVCTDSHTDMRPLSGGLIAFSTLDGRP SAHDFDNSPVLQDWVTATDIRVAFSRLHTFGDENEDDSELARDSYFYAVSDLQVGGR CKCNGHAARCVRDRDDSLVCDCRHNTAGPECDRCKPFHYDRPWQRATAREANECV ACNCNLHARRCRFNMELYKLSGRKSGGVCLNCRHNTAGRHCHYCKEGYYRDMGKPI THRKACKACDCHPVGAAGKTCNQTTGQCPCKDGVTGITCNRCAKGYQQSRSPIAPCI KIPVAPPTTAASSVEEPEDCDSYCKASKGKLKINMKKYCKKDYAVQIHILKADKAGDW WKFTVNIISVYKQGTSRIRRGDQSLWIRSRDIACKCPKIKPLKKYLLLGNAEDSPDQSGI VADKSSLVIQWRDTWARRLRKFQQREKKGKCKKA; this sequence can have the following modifications/variations. Glycosylation: amino acids 95-95 {N-linked (GlcNAc . . . ) asparagine}; amino acids 116-116 {N-linked (GlcNAc . . . ) asparagine}; amino acids 131-131 {N-linked (GlcNAc . . . ) asparagine}; amino acids 417-417 {N-linked (GlcNAc . . . ) asparagine}; Disulfide bond: amino acids 119-152; amino acids 285-294; amino acids 287-304; amino acids 306-315; amino acids 318-338; amino acids 341-350; amino acids 343-368; amino acids 371-380; amino acids 383-401; amino acids 404-416; amino acids 406-423; amino acids 425-434; amino acids 437-451; amino acids 472-544; amino acids 491-601; Sequence conflict: amino acids 299-299 {D->T}; Natural variant: amino acids 351-351 {in a neuroblastoma sample. R->H}; amino acids 489-489 {in a neuroblastoma sample. K->E}; Natural variant: amino acids 351-351 {in a neuroblastoma sample. R->H}; amino acids 489-489 {in a neuroblastoma sample. K->E}; The target “RGM domain family member B” has the sequence: [SEQ ID NO: 12] MGLRAAPSSAAAAAAEVEQRRSPGLCPPPLELLLLLLFSLGLLHAGDCQQPAQCRIQK CTTDFVSLTSHLNSAVDGFDSEFCKALRAYAGCTQRTSKACRGNLVYHSAVLGISDLM SQRNCSKDGPTSSTNPEVTHDPCNYHSHAGAREHRRGDQNPPSYLFCGLFGDPHLR TFKDNFQTCKVEGAWPLIDNNYLSVQVTNVPVVPGSSATATNKITIIFKAHHECTDQKV YQAVTDDLPAAFVDGTTSGGDSDAKSLRIVERESGHYVEMHARYIGTTVFVRQVGRYL TLAIRMPEDLAMSYEESQDLQLCVNGCPLSERIDDGQGQVSAILGHSLPRTSLVQAWP GYTLETANTQCHEKMPVKDIYFQSCVFDLLTTGDANFTAAAHSALEDVEALHPRKERW HIFPSSGNGTPRGGSDLSVSLGLTCLILIVFL; this sequence can have the following modifications/variations. Lipidation: amino acids 413-413 {GPI-anchor amidated asparagine}; Glycosylation: amino acids 120-120 {N-linked (GlcNAc . . . ) asparagine}; amino acids 383-383 {N-linked (GlcNAc . . . ) asparagine}; Disulfide bond: amino acids 139-226; amino acids 163-312; Sequence conflict: amino acids 22-22 {S->R}; amino acids 225-225 {E->G}; Mutagenesis: amino acids 186-186 {Severely impairs interaction with NEO1. A->R}; amino acids 206-206 {introduces a N-linked glycan %3B changes interaction with NEO1 from a 2:2 to a 1:1 stoichiometry. P->N};
The target “Collagen alpha-1(XVIII) chain” has the sequence:

[SEQ ID NO: 13]
MAPYPCGCHILLLLFCCLAAARANLLNLNWLWFNNEDTSHAATTIP
EPQGPLPVQPTADTTTHVTPRNGSTEPATAPGSPEPPSELLEDGQD
TPTSAESPDAPEENIAGVGAEILNVAKGIRSFVQLWNDTVPTESLA
RAETLVLETPVGPLALAGPSSTPQENGTTLWPSRGIPSSPGAHTTE
AGTLPAPTPSPPSLGRPWAPLTGPSVPPPSSGRASLSSLLGGAPPW
GSLQDPDSQGLSPAAAAPSQQLQRPDVRLRTPLLHPLVMGSLGKHA
APSAFSSGLPGALSQVAVTTLTRDSGAWVSHVANSVGPGLANNSAL
LGADPEAPAGRCLPLPPSLPVCGHLGISRFWLPNHLHHESGEQVRA
GARAWGGLLQTHCHPFLAWFFCLLLVPPCGSVPPPAPPPCCQFCEA
LQDACWSRLGGGRLPVACASLPTQEDGYCVLIGPAAERISEEVGLL
QLLGDPPPQQVTQTDDPDVGLAYVFGPDANSGQVARYHFPSLFFRD
FSLLFHIRPATEGPGVLFAITDSAQAMVLLGVKLSGVQDGHQDISL
LYTEPGAGQTHTAASFRLPAFVGQWTHLALSVAGGFVALYVDCEEF
ORMPLARSSRGLELEPGAGLFVAQAGGADPDKFQGVIAELKVRRDP
QVSPMHCLDEEGDDSDGASGDSGSGLGDARELLREETGAALKPRLP
APPPVTTPPLAGGSSTEDSRSEEVEEQTTVASLGAQTLPGSDSVST
WDGSVRTPGGRVKEGGLKGQKGEPGVPGPPGRAGPPGSPCLPGPPG
LPCPVSPLGPAGPALQTVPGPQGPPGPPGRDGTPGRDGEPGDPGED
GKPGDTGPQGFPGTPGDVGPKGDKGDPGVGERGPPGPQGPPGPPGP
SFRHDKLTFIDMEGSGFGGDLEALRGPRGFPGPPGPPGVPGLPGEP
GRFGVNSSDVPGPAGLPGVPGREGPPGFPGLPGPPGPPGREGPPGR
TGQKGSLGEAGAPGHKGSKGAPGPAGARGESGLAGAPGPAGPPGPP
GPPGPPGPGLPAGFDDMEGSGGPFWSTARSADGPQGPPGLPGLKGD
PGVPGLPGAKGEVGADGVPGFPGLPGREGIAGPQGPKGDRGSRGEK
GDPGKDGVGQPGLPGPPGPPGPVVYVSEQDGSVLSVPGPEGRPGFA
GFPGPAGPKGNLGSKGERGSPGPKGEKGEPGSIFSPDGGALGPAQK
GAKGEPGFRGPPGPYGRPGYKGEIGFPGRPGRPGMNGLKGEKGEPG
DASLGFGMRGMPGPPGPPGPPGPPGTPVYDSNVFAESSRPGPPGLP
GNQGPPGPKGAKGEVGPPGPPGQFPFDFLQLEAEMKGEKGDRGDAG
QKGERGEPGGGGFFGSSLPGPPGPPGPPGPRGYPGIPGPKGESIRG
QPGPPGPQGPPGIGYEGRQGPPGPPGPPGPPSFPGPHRQTISVPGP
PGPPGPPGPPGTMGASSGVRLWATRQAMLGQVHEVPEGWLIFVAEQ
EELYVRVQNGFRKVQLEARTPLPRGTDNEVAALQPPVVQLHDSNPY
PRREHPHPTARPWRADDILASPPRLPEPQPYPGAPHHSSYVHLRPA
RPTSPPAHSHRDFQPVLHLVALNSPLSGGMRGIRGADFQCFQQARA
VGLAGTFRAFLSSRLQDLYSIVRRADRAAVPIVNLKDELLFPSWEA
LFSGSEGPLKPGARIFSFDGKDVLRHPTWPQKSVWHGSDPNGRRLT
ESYCETWRTEAPSATGQASSLLGGRLLGQSAASCHHAYIVLCIENS
FMTA

SK; this sequence can have the following modifications/variations. Glycosylation: amino acids 68-68 {N-linked (GlcNAc . . . ) asparagine}; amino acids 129-129 {N-linked (GlcNAc . . . ) asparagine}; amino acids 164-164 {N-linked (GlcNAc . . . ) asparagine}; amino acids 926-926 {N-linked (GlcNAc . . . ) asparagine}; amino acids 1567-1567 {O-linked (GalNAc . . . ) threonine}; Disulfide bond: amino acids 334-397; amino acids 344-390; amino acids 381-419; amino acids 408-443; amino acids 412-432; amino acids 1604-1744; amino acids 1706-1736; Sequence conflict: amino acids 299-299 {R->K}; amino acids 663-663 {S->F}; amino acids 663-663 {S->F}; amino acids 1112-1112 {V->L}; amino acids 1147-1147 {P->R}; amino acids 1168-1168 {R->L}; amino acids 1210-1210 {P->L}; amino acids 1299-1299 {A->P}; amino acids 1319-1319 {L->K}; amino acids 1355-1355 {P->A}; amino acids 1358-1358 {P->A}; amino acids 1362-1364 {Missing}; amino acids 1362-1364 {Missing}; amino acids 1362-1364 {Missing}; amino acids 1362-1364 {Missing}; amino acids 1362-1364 {Missing}; amino acids 1362-1364 {Missing}; amino acids 1362-1364 {Missing}; amino acids 1362-1364 {Missing}; amino acids 1444-1444 {G->GQ}; amino acids 1542-1542 {R->G}; amino acids 1552-1552 {A->G}; amino acids 1561-1562 {LR->CG}; amino acids 1681-1681 {R->T}; amino acids 1685-1685 {W->R}; amino acids 1721-1721 {S->Y}; amino acids 1736-1736 {C->S}; Natural variant: amino acids 49-49 {Q->L}; amino acids 111-111 {G->R}; amino acids 288-288 {A->T}; amino acids 379-379 {T->M}; amino acids 1076-1076 {V->I}; amino acids 1121-1121 {P->R}; amino acids 1195-1195 {Q->H}; amino acids 1675-1675 {Functional polymorphism %3B associated with increased risk for prostate cancer %3B may cause Knobloch syndrome in compound heterozygotes carrying a frameshift/truncating mutation %3B results in decreased affinity for laminin. D->N}; Alternative sequence: amino acids 1-415 {in isoform 3. Missing}; amino acids 216-450 {in isoform 2. Missing}; amino acids 416-450 {in isoform 3. QDACWSRLGGGRLPVACASLPTQEDGYCVLIGPAA->MAPRCPWPWPRRRRLLDVLAPLVLLLGVRAASAEP}; Modified residue: amino acids 696-696 {Phosphothreonine}; Natural variant: amino acids 49-49 {Q->L}; amino acids 111-111 {G->R}; amino acids 288-288 {A->T}; amino acids 379-379 {T->M}; amino acids 1076-1076 {V->I}; amino acids 1121-1121 {P->R}; amino acids 1195-1195 {Q->H}; amino acids 1675-1675 {Functional polymorphism %3B associated with increased risk for prostate cancer %3B may cause Knobloch syndrome in compound heterozygotes carrying a frameshift/truncating mutation %3B results in decreased affinity for laminin. D->N};
The target “Tumor necrosis factor receptor superfamily member 1B” has the sequence:
[SEQ ID NO: 14] MAPVAVWAALAVGLELWAAAHALPAQVAFTPYAPEPGSTCRLREYYDQTAQMCCSK CSPGQHAKVFCTKTSDTVCDSCEDSTYTQLWNWVPECLSCGSRCSSDQVETQACTR EQNRICTCRPGWYCALSKQEGCRLCAPLRKCRPGFGVARPGTETSDVVCKPCAPGT FSNTTSSTDICRPHQICNVVAIPGNASMDAVCTSTSPTRSMAPGAVHLPQPVSTRSQH TQPTPEPSTAPSTSFLLPMGPSPPAEGSTGDFALPVGLIVGVTALGLLIIGVVNCVIMTQ VKKKPLCLQREAKVPHLPADKARGTQGPEQQHLLITAPSSSSSSLESSASALDRRAPT RNQPQAPGVEASGAGEARASTGSSDSSPGGHGTQVNVTCIVNVCSSSDHSSQCSSQ ASSTMGDTDSSPSESPKDEQVPFSKEECAFRSQLETPETLLGSTEEKPLPLGVPDAG MKPS; this sequence can have the following modifications/variations. Glycosylation: amino acids 30-30 {O-linked (GalNAc . . . ) threonine}; amino acids 171-171 {N-linked (GlcNAc . . . ) asparagine}; amino acids 193-193 {N-linked (GlcNAc . . . ) asparagine}; amino acids 206-206 {O-linked (GalNAc . . . ) threonine}; amino acids 221-221 {O-linked (GalNAc . . . ) serine}; amino acids 222-222 {O-linked (GalNAc . . . ) threonine}; Disulfide bond: amino acids 40-53; amino acids 54-67; amino acids 57-75; amino acids 78-93; amino acids 96-110; amino acids 100-118; amino acids 120-126; amino acids 134-143; amino acids 137-161; amino acids 164-179; Sequence conflict: amino acids 35-37 {EPG->APT}; amino acids 98-98 {S->P}; amino acids 102-102 {S->P}; amino acids 141-141 {R->P}; amino acids 363-363 {A->T}; Natural variant: amino acids 187-187 {V->M}; amino acids 196-196 {Frequent polymorphism %3B seems to be associated with hyperandrogenism %2C polycystic ovary syndrome (PCOS) and systemic lupus erythematosus. M->R}; amino acids 232-232 {E->K}; amino acids 236-236 {A->T}; amino acids 264-264 {L->P}; amino acids 269-269 {T->P}; amino acids 295-295 {Q->R}; amino acids 301-301 {P->R}; Alternative sequence: amino acids 263-268 {in isoform 2. GLIVGV->ASLACR}; amino acids 269-461 {in isoform 2. Missing}; Modified residue: amino acids 330-330 {Phosphoserine}; Natural variant: amino acids 187-187 {V->M}; amino acids 196-196 {Frequent polymorphism %3B seems to be associated with hyperandrogenism %2C polycystic ovary syndrome (PCOS) and systemic lupus erythematosus. M->R}; amino acids 232-232 {E->K}; amino acids 236-236 {A->T}; amino acids 264-264 {L->P}; amino acids 269-269 {T->P}; amino acids 295-295 {Q->R}; amino acids 301-301 {P->R};
The target “Beta-2-microglobulin” has the sequence:
[SEQ ID NO: 15] MSRSVALAVLALLSLSGLEAIQRTPKIQVYSRHPAENGKSNFLNCYVSGFHPSDIEVDL LKNGERIEKVEHSDLSFSKDWSFYLLYYTEFTPTEKDEYACRVNHVTLSQPKIVKWDR DM; this sequence can have the following modifications/variations. Glycosylation: amino acids 21-21 {N-linked (Glc) (glycation) isoleucine %3B in hemodialysis-associated amyloidosis}; amino acids 39-39 {N-linked (Glc) (glycation) lysine %3B in vitro}; amino acids 61-61 {N-linked (Glc) (glycation) lysine %3B in vitro}; amino acids 68-68 {N-linked (Glc) (glycation) lysine %3B in vitro}; amino acids 78-78 {N-linked (Gc) (glycation) lysine %3B in vitro}; amino acids 111-111 {N-linked (Gc) (glycation) lysine %3B in vitro}; amino acids 114-114 {N-linked (Glc) (glycation) lysine %3B in vitro}; Disulfide bond: amino acids 45-100; Sequence conflict: amino acids 20-20 {A->G}; amino acids 52-52 {P->Q}; amino acids 119-119 {M->1}; Natural variant: amino acids 11-11 {in IMD43%3B lower levels of B2M %2C MHC class I and FCGRT proteins. A->P}; amino acids 96-96 {in AMYL8%3B reduced stability %3B in contrast to the wild-type %2C the mutant aggregates into fibrils with classic amyloid-like properties under physiologic solvent conditions. D->N}; Modified residue: amino acids 22-22 {Pyrrolidone carboxylic acid %3B in form μl 5.3}; Mutagenesis: amino acids 79-79 {Increases tendency towards amyloid formation. D->P}; amino acids 80-80 {Decreases tendency towards amyloid formation. W->G}; amino acids 80-80 {Increases tendency towards amyloid formation. W->V}; Natural variant: amino acids 11-11 {in IMD43%3B lower levels of B2M %2C MHC class I and FCGRT proteins. A->P}; amino acids 96-96 {in AMYL8%3B reduced stability %3B in contrast to the wild-type %2C the mutant aggregates into fibrils with classic amyloid-like properties under physiologic solvent conditions. D->N};
The target “Secreted frizzled-related protein 3” has the sequence:
[SEQ ID NO: 16] MVCGSPGGMLLLRAGLLALAALCLLRVPGARAAACEPVRIPLCKSLPWNMTKMPNHL HHSTQANAILAIEQFEGLLGTHCSPDLLFFLCAMYAPICTIDFQHEPIKPCKSVCERARQ GCEPILIKYRHSWPENLACEELPVYDRGVCISPEAIVTADGADFPMDSSNGNCRGASS ERCKCKPIRATQKTYFRNNYNYVIRAKVKEIKTKCHDVTAVVEVKEILKSSLVNIPRDTV NLYTSSGCLCPPLNVNEEYIIMGYEDEERSRLLLVEGSIAEKWKDRLGKKVKRWDMKL RHLGLSKSDSSNSDSTQSQKSGRNSNPRQARN; this sequence can have the following modifications/variations. Glycosylation: amino acids 49-49 {N-linked (GlcNAc . . . ) asparagine}; Disulfide bond: amino acids 35-96; amino acids 43-89; amino acids 80-119; amino acids 108-147; amino acids 112-136; Sequence conflict: amino acids 63-63 {A->D}; amino acids 106-106 {K->N}; Natural variant: amino acids 200-200 {R->W}; amino acids 324-324 {in OS1%3B associated with disease susceptibility %3B has diminished ability to antagonize Wnt signaling %2C in vitro. R->G}; Natural variant: amino acids 200-200 {R->W}; amino acids 324-324 {in OS1%3B associated with disease susceptibility %3B has diminished ability to antagonize Wnt signaling %2C in vitro. R->G};
The target “Coiled-coil domain-containing protein 80” has the sequence:
[SEQ ID NO: 17] MTWRMGPRFTMLLAMWLVCGSEPHPHATIRGSHGGRKVPLVSPDSSRPARFLRHTG RSRGIERSTLEEPNLQPLQRRRSVPVLRLARPTEPPARSDINGAAVRPEQRPAARGSP REMIRDEGSSARSRMLRFPSGSSSPNILASFAGKNRVWVISAPHASEGYYRLMMSLLK DDVYCELAERHIQQIVLFHQAGEEGGKVRRITSEGQILEQPLDPSLIPKLMSFLKLEKGK FGMVLLKKTLQVEERYPYPVRLEAMYEVIDQGPIRRIEKIRQKGFVQKCKASGVEGQV VAEGNDGGGGAGRPSLGSEKKKEDPRRAQVPPTRESRVKVLRKLAATAPALPQPPST PRATTLPPAPATTVTRSTSRAVTVAARPMTTTAFPTTQRPWTPSPSHRPPTTTEVITAR RPSVSENLYPPSRK DQHRERPQTTRRPSKATSLESFTNAPPTTISEPSTRAAGPGRFRDNRMDRREHGHRD PNVVPGPPKPAKEKPPKKKAQDKILSNEYEEKYDLSRPTASQLEDELQVGNVPLKKAK ESKKHEKLEKPEKEKKKKMKNENADKLLKSEKQMKKSEKKSKQEKEKSKKKKGGKTE QDGYQKPTNKHFTQSPKKSVADLLGSFEGKRRLLLITAPKAENNMYVQQRDEYLESFC KMATRKISVITIFGPVNNSTMKIDHFQLDNEKPMRVVDDEDLVDQRLISELRKEYGMTY NDFFMVLTDVDLRVKQYYEVPITMKSVFDLIDTFQSRIKDMEKQKKEGIVCKEDKKQSL ENFLSRFRWRRRLLVISAPNDEDWAYSQQLSALSGQACNFGLRHITILKLLGVGEEVG GVLELFPINGSSVVEREDVPAHLVKDIRNYFQVSPEYFSMLLVGKDGNVKSWYPSPM WSMVIVYDLIDSMQLRRQEMAIQQSLGMRCPEDEYAGYGYHSYHQGYQDGYQDDYR HHESYHHGYPY; this sequence can have the following modifications/variations. Sequence conflict: amino acids 411-411 {S->L}; amino acids 549-549 {E->K}; amino acids 551-551 {K->E}; amino acids 837-837 {S->G}; Alternative sequence: amino acids 1-865 {in isoform 3. Missing}; amino acids 1-1 {in isoform 2. M->MTSVHRKVDYTM}; Cross-link: amino acids 545-545 {Glycyl lysine isopeptide (Lys-Gly) (interchain with G-Cter in SUMO2)}; amino acids 548-548 {Glycyl lysine isopeptide (Lys-Gly) (interchain with G-Cter in SUMO2)};
The target “Chordin-like protein 1” has the sequence:
[SEQ ID NO: 18] MGGMKYIFSLLFFLLLEGGKTEQVKHSETYCMFQDKKYRVGERWHPYLEPYGLVYCV NCICSENGNVLCSRVRCPNVHCLSPVHIPHLCCPRCPDSLPPVNNKVTSKSCEYNGTT YQHGELFVAEGLFQNRQPNQCTQCSCSEGNVYCGLKTCPKLTCAFPVSVPDSCCRV CRGDGELSWEHSDGDIFRQPANREARHSYHRSHYDPPPSRQAGGLSRFPGARSHR GALMDSQQASGTIVQIVINNKHKHGQVCVSNGKTYSHGESWHPNLRAFGIVECVLCTC NVTKQECKKIHCPNRYPCKYPQKIDGKCCKVCPGKKAKELPGQSFDNKGYFCGEETM PVYESVFMEDGETTRKIALETERPPQVEVHVWTIRKGILQHFHIEKISKRMFEELPHFKL VTRTTLSQWKIFTEGEAQISQMCSSRVCRTELEDLVKVLYLERSEKGHC; this sequence can have the following modifications/variations. Glycosylation: amino acids 112-112 {N-linked (GlcNAc . . . ) asparagine}; amino acids 285-285 {N-linked (GlcNAc . . . ) asparagine}; Natural variant: amino acids 254-254 {in MGC1. C->F}; Alternative sequence: amino acids 1-1 {in isoform 3%2C isoform 4%2C isoform 5 and isoform 6. M->MRKKWKM}; amino acids 95-173 {in isoform 3. Missing}; amino acids 95-95 {in isoform 2 and isoform 4. D->ED}; amino acids 323-323 {in isoform 2%2C isoform 3%2C isoform 4 and isoform 5. E->EE}; Natural variant: amino acids 254-254 {in MGC1. C->F};
The target “Neuroblastoma suppressor of tumorigenicity 1” has the sequence:
[SEQ ID NO: 19] MMLRVLVGAVLPAMLLAAPPPINKLALFPDKSAWCEAKNITQIVGHSGCEAKSIQNRAC LGQCFSYSVPNTFPQSTESLVHCDSCMPAQSMWEIVTLECPGHEEVPRVDKLVEKILH CSCQACGKEPSHEGLSVYVQGEDGPGSQPGTHPHPHPHPHPGGQTPEPEDPPGAP HTEEEGAE D; this sequence can have the following modifications/variations. Disulfide bond: amino acids 35-85; amino acids 49-99; amino acids 59-118; amino acids 63-120; amino acids 82-123; Alternative sequence: amino acids 1-1 {in isoform 2. M->MPGNLMSQTSRAVSIWKFPAKLGKTHGHRALEATGM};
The target “Kallikrein-11” has the sequence:
[SEQ ID NO: 20] MQRLRWLRDWKSSGRGLTAAKEPGARSSPLQAMRILQLILLALATGLVGGETRIIKGFE CKPHSQPWQAALFEKTRLLCGATLIAPRWLLTAAHCLKPRYIVHLGQHNLQKEEGCEQ TRTATESFPHPGFNNSLPNKDHRNDIMLVKMASPVSITWAVRPLTLSSRCVTAGTSCLI SGWGSTSSPQLRLPHTLRCANITIIEHQKCENAYPGNITDTMVCASVQEGGKDSCQGD SGGPLVCNQSLQGIISWGQDPCAITRKPGVYTKVCKYVDWIQETMKNN; this sequence can have the following modifications/variations. Glycosylation: amino acids 131-131 {N-linked (GlcNAc . . . ) asparagine}; amino acids 197-197 {N-linked (GlcNAc . . . ) asparagine}; amino acids 213-213 {N-linked (GlcNAc . . . ) asparagine}; amino acids 242-242 {N-linked (GlcNAc . . . ) asparagine}; Disulfide bond: amino acids 60-195; amino acids 79-95; amino acids 167-269; amino acids 174-241; amino acids 206-220; amino acids 231-256; Natural variant: amino acids 32-32 {A->T}; amino acids 49-49 {G->E}; amino acids 166-166 {R->C}; Alternative sequence: amino acids 1-32 {in isoform 1 and isoform 4. Missing}; amino acids 98-98 {in isoform 3 and isoform 4. P->PWVSLTSPTHVSPDLSSSNYCLSHLS}; Natural variant: amino acids 32-32 {A->T}; amino acids 49-49 {G->E}; amino acids 166-166 {R->C};
The target “Pleiotrophin” has the sequence:

[SEQ ID NO: 21]
MQAQQYQQQRRKFAAAFLAFIFILAAVDTAEAGKKEKPEKKVKKSD
CGEWQWSVCVPTSGDCGLGTREGTRTGAECKQTMKTQRCKIPCNWK
KQFGAECKYQFQAWGECDLNTALKTRTGSLKRALHNAECQKTVTIS
KPCGKLTKPKPQAESKKKKKEGKKQEKMLD;

this sequence can have the following modifications/variations. Disulfide bond: amino acids 47-76; amino acids 55-85; amino acids 62-89; amino acids 99-131; amino acids 109-141; Sequence conflict: amino acids 42-42 {V->K}; amino acids 47-47 {C->D}; amino acids 75-75 {E->G};
The target “Secreted frizzled-related protein 1” has the sequence:
[SEQ ID NO: 22] MGIGRSEGGRRGAALGVLLALGAALLAVGSASEYDYVSFQSDIGPYQSGRFYTKPPQ CVDIPADLRLCHNVGYKKMVLPNLLEHETMAEVKQQASSWVPLLNKNCHAGTQVFLC SLFAPVCLDRPIYPCRWLCEAVRDSCEPVMQFFGFYWPEMLKCDKFPEGDVCIAMTP PNATEASKPQGTTVCPPCDNELKSEAIIEHLCASEFALRMKIKEVKKENGDKKIVPKKKK PLKLGPIKKKDLKKLVLYLKNGADCPCHQLDNLSHHFLIMGRKVKSQYLLTAIHKWDKK NKEFKNFMKKMKNHECPTFQSVFK; this sequence can have the following modifications/variations. Glycosylation: amino acids 173-173 {N-linked (GlcNAc . . . ) asparagine}; Disulfide bond: amino acids 58-121; amino acids 68-114; amino acids 105-140; amino acids 129-166; amino acids 133-157; amino acids 186-256; amino acids 189-258; amino acids 203-306; Sequence conflict: amino acids 14-14 {Missing}; amino acids 14-14 {Missing}; amino acids 174-174 {A->P}; Mutagenesis: amino acids 173-173 {Reduced molecular weight. N->Q}; amino acids 263-263 {No effect on molecular weight. N->Q};
The target “Growth/differentiation factor 15” has the sequence:
[SEQ ID NO: 23]MPGQELRTVNGSQMLLVLLVLSWLPHGGALSLAEASRASFPGPSELHSEDSRFRELR KRYEDLLTRLRANQSWEDSNTDLVPAPAVRILTPEVRLGSGGHLHLRISRAALPEGLPE ASRLHRALFRLSPTASRSWDVTRPLRRQLSLARPQAPALHLRLSPPPSQSDQLLAESS SARPQLELHLRPQAARGRRRARARNGDHCPLGPGRCCRLHTVRASLEDLGWADWVL SPREVQVTMCIGACPSQFRAANMHAQIKTSLHRLKPDTVPAPCCVPASYNPMVLIQKT DTGVSLQTYDDLLAKDCHCI; this sequence can have the following modifications/variations. Glycosylation: amino acids 70-70 {N-linked (GlcNAc . . . ) asparagine}; Disulfide bond: amino acids 203-210; amino acids 211-274; amino acids 240-305; amino acids 244-307; amino acids 273-273 {Interchain}; Sequence conflict: amino acids 269-269 {V->E}; amino acids 288-288 {T->A}; Natural variant: amino acids 9-9 {V->L}; amino acids 48-48 {S->T}; amino acids 202-202 {H->D}; Mutagenesis: amino acids 225-225 {No effect on interaction with GFRAL. Attenuates GDF15-mediated food-intake inhibition. W->A}; amino acids 283-283 {Reduces cellular signaling mediated by GFRAL and RET. V->A}; amino acids 283-283 {Abolishes interaction with GFRAL. Abolishes RET phosphorylation and cellular signaling mediated by GFRAL and RET. V->R}; amino acids 285-285 {Reduces cellular signaling mediated by GFRAL and RET. Abolishes interaction with GFRAL and GDF15-mediated food-intake inhibition. I->A}; Natural variant: amino acids 9-9 {V->L}; amino acids 48-48 {S->T}; amino acids 202-202 {H->D};
The target “Periostin” has the sequence:
[SEQ ID NO: 24] MIPFLPMFSLLLLLIVNPINANNHYDKILAHSRIRGRDQGPNVCALQQILGTKKKYFSTCK NWYKKSICGQKTTVLYECCPGYMRMEGMKGCPAVLPIDHVYGTLGIVGATTTQRYSD ASKLREEIEGKGSFTYFAPSNEAWDNLDSDIRRGLESNVNVELLNALHSHMINKRMLTK DLKNGMIIPSMYNNLGLFINHYPNGVVTVNCARIIHGNQIATNGVVHVIDRVLTQIGTSIQ DFIEAEDDLSSFRAAAITSDILEALGRDGHFTLFAPTNEAFEKLPRGVLERIMGDKVASE ALMKYHILNTLQCSESIMGGAVFETLEGNTIEIGCDGDSITVNGIKMVNKKDIVTNNGVIH LIDQVLIPDSAKQVIELAGKQQTTFTDLVAQLGLASALRPDGEYTLLAPVNNAFSDDTLS MDQRLLKLILQNHILKVKVGLNELYNGQILETIGGKQLRVFVYRTAVCIENSCMEKGSKQ GRNGAIHIFREIIKPAEKSLHEKLKQDKRFSTFLSLLEAADLKELLTQPGDWTLFVPTND AFKGMTSEEKEILIRDKNALQNIILYHLTPGVFIGKGFEPGVTNILKTTQGSKIFLKEVNDT LLVNELKSKESDIMTTNGVIHVVDKLLYPADTPVGNDQLLEILNKLIKYIQIKFVRGSTFKE IPVTVYTTKIITKVVEPKIKVIEGSLQPIIKTEGPTLTKVKIEGEPEFRLIKEGETITEVIHGEP IIKKYTKIIDGVPVEITEKETREERIITGPEIKYTRISTGGGETEETLKKLLQEEVTKVTKFIE GGDGHLFEDEEIKRLLQGDTPVRKLQANKKVQGSRRRLREGRSQ; this sequence can have the following modifications/variations. Glycosylation: amino acids 599-599 {N-linked (GlcNAc . . . ) asparagine}; Disulfide bond: amino acids 44-80; amino acids 60-69; amino acids 79-92; Sequence conflict: amino acids 290-290 {I->F}; amino acids 290-290 {I->F}; amino acids 290-290 {I->F}; amino acids 290-290 {I->F}; amino acids 290-290 {I->F}; amino acids 290-290 {I->F}; amino acids 421-421 {D->V}; amino acids 421-421 {D->V}; amino acids 421-421 {D->V}; amino acids 421-421 {D->V}; amino acids 421-421 {D->V}; amino acids 421-421 {D->V}; Natural variant: amino acids 339-339 {T->I}; amino acids 814-814 {V->M}; Alternative sequence: amino acids 670-757 {in isoform 6 and isoform 7. TTKIITKVVEPKIKVIEGSLQPIIKTEGPTLTKVKIEGEPEFRLIKEGETITEVIHGEPIIKKYT KIIDGVPVEITEKETREERIITG->S}; amino acids 670-727 {in isoform 2 and isoform 4. TTKIITKVVEPKIKVIEGSLQPIIKTEGPTLTKVKIEGEPEFRLIKEGETITEVIHGE->K}; amino acids 670-697 {in isoform 3 and isoform 5. TTKIITKVVEPKIKVIEGSLQPIIKTEG->R}; amino acids 783-810 {in isoform 3%2C isoform 4 and isoform 7. Missing}; Natural variant: amino acids 339-339 {T->I}; amino acids 814-814 {V->M};
The target “CD59 glycoprotein” has the sequence:
[SEQ ID NO: 25] MGIQGGSVLFGLLLVLAVFCHSGHSLQCYNCPNPTADCKTAVNCSSDFDACLITKAGL QVYNKCWKFEHCNFNDVTTRLRENELTYYCCKKDLCNFNEQLENGGTSLSEKTVLLL VTPFLAAAWSLHP; this sequence can have the following modifications/variations. Lipidation: amino acids 102-102 {GPI-anchor amidated asparagine}; Glycosylation: amino acids 43-43 {N-linked (GlcNAc . . . ) asparagine}; amino acids 66-66 {N-linked (Glc) (glycation) lysine}; amino acids 76-76 {O-linked (GalNAc . . . ) threonine}; amino acids 77-77 {O-linked (GalNAc . . . ) threonine}; Disulfide bond: amino acids 28-51; amino acids 31-38; amino acids 44-64; amino acids 70-88; amino acids 89-94; Natural variant: amino acids 89-89 {in HACD59. C->Y}; Mutagenesis: amino acids 29-29 {No loss of function. Y->R}; amino acids 33-33 {No loss of function. N->R %2CQ}; amino acids 37-37 {No loss of function. D->R}; amino acids 48-48 {Some loss of function. Some lysis. F->R}; amino acids 49-49 {Loss of function. Lysis. D->R}; amino acids 58-58 {No loss of function. L->E}; amino acids 63-63 {No loss of function. K->E}; amino acids 65-65 {Complete loss of function. Lysis. W->E}; amino acids 66-66 {No loss of function. K->D}; amino acids 66-66 {Loss of glycation mediated inactivation. K->Q}; amino acids 67-67 {No loss of function. F->K}; amino acids 69-69 {Loss of glycation mediated inactivation. H->Q}; amino acids 72-72 {Almost complete loss of function. Lysis. F->E}; amino acids 78-78 {Loss of function. Lysis. R->E}; amino acids 79-79 {No loss of function. L->D}; amino acids 81-81 {Almost complete loss of function. Lysis. E->R}; amino acids 82-82 {No loss of function. N->K}; amino acids 87-87 {No loss of function. Y->R}; Natural variant: amino acids 89-89 {in HACD59. C->Y};
The target “Tumor necrosis factor receptor superfamily member 19L” has the sequence:
[SEQ ID NO: 26] MKPSLLCRPLSCFLMLLPWPLATLTSTTLWQCPPGEEPDLDPGQGTLCRPCPPGTFS AAWGSSPCQPHARCSLWRRLEAQVGMATRDTLCGDCWPGWFGPWGVPRVPCQPC SWAPLGTHGCDEWGRRARRGVEVAAGASSGGETRQPGNGTRAGGPEETAAQYAVI AIVPVFCLMGLLGILVCNLLKRKGYHCTAHKEVGPGPGGGGSGINPAYRTEDANEDTIG VLVRLITEKKENAAALEELLKEYHSKQLVQTSHRPVSKLPPAPPNVPHICPHRHHLHTV QGLASLSGPCCSRCSQKKWPEVLLSPEAVAATTPVPSLLPNPTRVPKAGAKAGRQGE ITILSVGRFRVARIPEQRTSSMVSEVKTITEAGPSWGDLPDSPQPGLPPEQQALLGSGG SRTKWLKPPAENKAEENRYVVRLSESNLVI; this sequence can have the following modifications/variations. Glycosylation: amino acids 149-149 {N-linked (GlcNAc . . . ) asparagine}; Disulfide bond: amino acids 51-65; amino acids 71-90; Sequence conflict: amino acids 122-122 {D->S}; amino acids 187-187 {K->E}; amino acids 273-273 {H->R}; Natural variant: amino acids 332-332 {A->V}; Modified residue: amino acids 223-223 {Phosphothreonine}; Natural variant: amino acids 332-332 {A->V};
The target “Cystatin-C” has the sequence:
[SEQ ID NO: 27] MAGPLRAPLLLLAILAVALAVSPAAGSSPGKPPRLVGGPMDASVEEEGVRRALDFAVG EYNKASNDMYHSRALQVVRARKQIVAGVNYFLDVELGRTTCTKTQPNLDNCPFHDQP HLKRK AFCSFQIYAVPWQGTMTLSKSTCQDA; this sequence can have the following modifications/variations. Disulfide bond: amino acids 99-109; amino acids 123-143; Natural variant: amino acids 25-25 {in ARMD11%3B alters processing and glycosylation. A->T}; amino acids 94-94 {in AMYL6. L->Q}; Modified residue: amino acids 43-43 {Phosphoserine %3B by FAM20C}; Mutagenesis: amino acids 25-25 {Shows a dual distribution to the Golgi apparatus and to the mitochondria. A->S}; Natural variant: amino acids 25-25 {in ARMD11%3B alters processing and glycosylation. A->T}; amino acids 94-94 {in AMYL6. L->Q};

In a preferred embodiment, the binding element(s) solely and selectively bind(s) a protein selected from the group CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

The term “solely” means that the binding element(s) bind(s) exclusively to a protein as defined above, i.e. the binding element(s) do(does) not bind to proteins other than a protein selected from the group CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB. In other words, in a preferred embodiment, the device or lumen as provided and defined herein does not comprise binding element binding to a protein other than a protein selected from the group CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In a preferred embodiment of the binding device reducing selectively at least two Targets is a device, comprising a platform associated with a first group of binding elements and a second group of binding elements, wherein each of the first group and second group of binding elements solely and selectively bind(s) a protein selected from the group: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein the first group of binding elements are selective for a different protein than the second group of binding elements.

In some embodiments, the binding element is selected from proteins, polymers, aptamers, peptides, viruses, nanoparticles, small molecules, a macrocycle compound, a polypeptide, a peptidomimetic compound, a nucleic acid, a nucleic acid analog and any other molecule. In some embodiments, the binding element comprises a protein. In some embodiments, the binding element comprises a polymer. In some embodiments, the binding element comprises an aptamer. In some embodiments, the binding element comprises a peptide. In some embodiments, the binding element comprises a virus. In some embodiments, the binding element comprises a small molecule. In some embodiments, the binding element comprises an antibody. In some embodiments, the binding element comprises a synthetic or natural antibody. In some embodiments, the binding element comprises recombinant antibodies, including but not limited to scFv, Fab fragments, diabodies. In some embodiments, recombinant antibodies are human or in some other embodiments—humanized. In some embodiments, the binding element is selected from a monoclonal antibody or polyclonal antibody. In some embodiments, the binding element is selected from a humanized monoclonal antibody or humanized polyclonal antibody. In some embodiments, the binding element comprises an antibody mimetic. In preferred embodiments, the binding element comprises an antibody, peptide or aptamer.

In some embodiments, the binding element selectively binds the protein with a KD of less than 1×10-3M. In some embodiments, the binding element selectively binds the protein with a KD of less than 1×10-4 M. In some embodiments, the binding element selectively binds the protein with a KD of less than 1×10-5 M. In some embodiments, the binding element selectively binds the protein with a KD of less than 1×10-6 M. In some embodiments, the binding element selectively binds the protein with a KD of less than 1×10-7 M. In some embodiments, the binding element selectively binds the protein with a KD of less than 1×10-8 M. In some embodiments, the binding element selectively binds the protein with a K_{D} of less than 1×10⁻⁹ M. In some embodiments, the binding element selectively binds the protein with a K_{D} of less than 1×10⁻¹⁰ M. In some embodiments, the binding element selectively binds the protein with a K_{D} of less than 1×10⁻¹² M. In some embodiments, the binding element selectively binds the protein with a K_{D} of less than 1×10⁻¹⁵ M. In certain embodiments, the binding element selectively binds the protein with a K_{D} of about 1×10⁻³M to about 1×10⁻¹⁵M. In certain embodiments, the binding element selectively binds the protein with a K_{D} of about 1×10⁻³M to about 1×10⁻¹⁴M. In certain embodiments, the binding element selectively binds the protein with a K_{D} of about 1×10⁻³M to about 1×10⁻¹³M. In certain embodiments, the binding element selectively binds the protein with a K_{D} of about 1×10⁻³M to about 1×10⁻¹²M. In certain embodiments, the binding element selectively binds the protein with a K_{D} of about 1×10⁻³M to about 1×10⁻¹¹M. In certain embodiments, the binding element selectively binds the protein with a K_{D} of about 1×10⁻³M to about 1×10⁻¹⁰M. In certain embodiments, the binding element selectively binds the protein with a K_{D} of about 1×10⁻³M to about 1×10⁻⁹M. In certain embodiments, the binding element selectively binds the protein with a K_{D} of about 1×10⁻³M to about 1×10⁻⁸M. In certain embodiments, the binding element selectively binds the protein with a K_{D} of about 1×10⁻³M to about 1×10⁻⁷M. In certain embodiments, the binding element selectively binds the protein with a K_{D} of about 1×10⁻³M to about 1×10⁻⁶M. In certain embodiments, the binding element selectively binds the protein with a K_{D} of about 1×10⁻⁶M to about 1×10⁻¹⁵M. In certain embodiments, the binding element selectively binds the protein with a K_{D} of about 1×10⁻⁹M to about 1×10⁻¹⁵M. In certain embodiments, the binding element selectively binds the protein with a K_{D} of about 1×10⁻¹²M to about 1×10⁻¹⁵M. In certain embodiments, the binding element selectively binds the protein with a K_{D} of about 1×10⁻⁶M to about 1×10⁻¹²M. In certain embodiments, the binding element selectively binds the protein with a K_{D} of about 1×10⁻⁶M to about 1×10⁻⁹M. In certain embodiments, such protein is selected from Targets.

In some embodiments, the binding element comprises a protein. In some embodiments, the binding element comprises an enzyme. In some embodiments, the enzyme modifies or degrades a selected protein. In some embodiments, the binding element comprises a protease. In some embodiments, the binding element comprises a hydrolase. In some embodiments, the binding element comprises a lyase. In some embodiments, the binding element comprises a ligase.

In some embodiments, the binding element comprises an antibody. In some embodiments, the binding element comprises an anti-CCDC80 antibody. In some embodiments, the binding element comprises an anti-CCDC80 human antibody. In some embodiments, the binding element comprises an anti-CD59 antibody. In some embodiments, the binding element comprises an anti-CD59 human antibody. In some embodiments, the binding element comprises an anti-CHRDL1 antibody. In some embodiments, the binding element comprises an anti-CHRDL1 human antibody. In some embodiments, the binding element comprises an anti-COL18A1 antibody. In some embodiments, the binding element comprises an anti-COL18A1 human antibody. In some embodiments, the binding element comprises an anti-CST3 antibody. In some embodiments, the binding element comprises an anti-CST3 human antibody. In some embodiments, the binding element comprises an anti-DPT antibody. In some embodiments, the binding element comprises an anti-DPT human antibody. In some embodiments, the binding element comprises an anti-EFEMP1 antibody. In some embodiments, the binding element comprises an anti-EFEMP1 human antibody. In some embodiments, the binding element comprises an anti-FAS antibody. In some embodiments, the binding element comprises an anti-FAS human antibody. In some embodiments, the binding element comprises an anti-FSTL3 antibody. In some embodiments, the binding element comprises an anti-FSTL3 human antibody. In some embodiments, the binding element comprises an anti-GAS1 antibody. In some embodiments, the binding element comprises an anti-GAS1 human antibody. In some embodiments, the binding element comprises an anti-GDF15 antibody. In some embodiments, the binding element comprises an anti-GDF15 human antibody. In some embodiments, the binding element comprises an anti-KLK11 antibody. In some embodiments, the binding element comprises an anti-KLK11 human antibody. In some embodiments, the binding element comprises an anti-MMP7 antibody. In some embodiments, the binding element comprises an anti-MMP7 human antibody. In some embodiments, the binding element comprises an anti-NBL1 antibody. In some embodiments, the binding element comprises an anti-NBL1 human antibody. In some embodiments, the binding element comprises an anti-NTN1 antibody. In some embodiments, the binding element comprises an anti-NTN1 human antibody. In some embodiments, the binding element comprises an anti-POSTN antibody. In some embodiments, the binding element comprises an anti-POSTN human antibody. In some embodiments, the binding element comprises an anti-PTN antibody. In some embodiments, the binding element comprises an anti-PTN human antibody. In some embodiments, the binding element comprises an anti-RELT antibody. In some embodiments, the binding element comprises an anti-RELT human antibody. In some embodiments, the binding element comprises an anti-SFRP1 antibody. In some embodiments, the binding element comprises an anti-SFRP1 human antibody. In some embodiments, the binding element comprises an anti-SMOC1 antibody. In some embodiments, the binding element comprises an anti-SMOC1 human antibody. In some embodiments, the binding element comprises an anti-STC1 antibody. In some embodiments, the binding element comprises an anti-STC1 human antibody. In some embodiments, the binding element comprises an anti-TNFRSF1A antibody. In some embodiments, the binding element comprises an anti-TNFRSF1A human antibody. In some embodiments, the binding element comprises an anti-UNC5C antibody. In some embodiments, the binding element comprises an anti-UNC5C human antibody. In some embodiments, the binding element comprises an anti-sFRP-3 antibody. In some embodiments, the binding element comprises an anti-sFRP-3 human antibody. In some embodiments, the binding element comprises an anti-TNFRSF1B antibody. In some embodiments, the binding element comprises an anti-TNFRSF1B human antibody. In some embodiments, the binding element comprises an anti-CD55 antibody. In some embodiments, the binding element comprises an anti-CD55 human antibody. In some embodiments, the binding element comprises an anti-BMP4 antibody. In some embodiments, the binding element comprises an anti-BMP4 human antibody. In some embodiments, the binding element comprises an anti-RGMB antibody. In some embodiments, the binding element comprises an anti-RGMB human antibody. In some embodiments, the binding element comprises an anti-GDF15 human antibody, an anti-FSTL3 human antibody, an anti-BMP4 human antibody or an anti-FRZB (sFRP3) human antibody.

Some of CHRDL1 binding elements. In some embodiments, the binding element comprises an element selected from: SOMAmer SL009400 from SomaLogic Inc (somalogic.com/); anti-CHRDL1 antibody [MK1] produced in mouse (Cat #SAB1408184), anti-CHRDL1 antibody produced in rabbit (Cat #HPA000226), anti-CHRDL1 antibody (Cat #HPA000250), anti-CHRDL1 antibody produced in rabbit (Cat #SAB1408696), monoclonal anti-CHRDL1 antibody produced in mouse (Cat #WH0091851M1) from Sigma-Aldrich (https://www.sigmaaldrich.com); anti-CHRDL1 antibody (Cat #ab103369) from Abcam (http://www.abcam.com); Human chordin like-1 antibody (Cat #MAB1808) from R&D systems (https://www.rndsystems.com); CHRDL1 antibody (Cat #MAB18) from NovusBio (https://www.novusbio.com); Anti-CHRDL1 Antibody (Cat #HPA000250) from Atlas Antibodies (https://atlasantibodies.com/); Anti-CHRDL1 Antibody (aa2-30) (Cat #LS-C168749) from LifeSpan BioSciences (https://www.isbio.com); CHRDL1 Antibody—N-terminal region (Cat #OAAB00684) from Aviva Systems Biology (https://www.avivasysbio.com); CHRDL1 antibody (Cat #orb28585) from Biorbyt (http://www.biorbyt.com); CHRDL1 Antibody (Cat #40398) from Signalway Antibody LLC (https://www.sabbiotech.com); CHRDL1 Monoclonal Antibody (GL4) (Cat #MA5-24380) from Invitrogen Antibodies (https://www.thermofisher.com); CHRDL1 Mouse Monoclonal Antibody (Cat #TA346918) from OriGene Technologies (https://www.origene.com); CHRDL1 polyclonal antibody (A01) (Cat #H00091851-A01) from Abnova Corporation (http://www.abnova.com); Anti-CHRDL1 Antibody, clone 10C9.3 (Cat #MABN453) from Merck (https://www.sigmaaldrich.com/); Anti-CHRDL1/Chordin Like 1 Monoclonal Antibody (Cat #A08014) from BosterBio (https://www.bosterbio.com).

Some of Cystatin C binding elements. In some embodiments, the binding element comprises an element selected from: SOMAmer SL001777 from SomaLogic Inc (somalogic.com/); Mouse Cystatin C Antibody (Cat #AF1238) from R&D Systems (http://www.biocompare.com/); Anti-Cystatin C antibody (Cat #ab97730), Anti-Cystatin C antibody—C-terminal (Cat #ab227279) and Anti-Cystatin C antibody (Cat #ab196526) from Abcam (http://www.abcam.com/); Rabbit Anti-Cystatin C Antibody (Cat #222040) from United States Biological (http://www.biocompare.com/); Mouse Anti-Human CST3 from Raybiotech (Cat #101-10350) fromRaybiotech (http://www.biocompare.com); Monoclonal Antibody to Cystatin (Cat #v MAB236P) from Maine Biotechnology Services (http://www.biocompare.com); Cystatin C (D6U3E) Rabbit mAb (Cat #24840) from Cell Signalling Technologies (https://www.cellsignal.com/); Anti-CST3 Antibody HPA013143 from Atlas Antibodies (https://atlasantibodies.com).

Some of FSTL3 binding elements. In some embodiments, the binding element comprises an element selected from: SOMAmer SL009324 from SomaLogic Inc (somalogic.com/); FSTL3 antibody (follistatin-Like 3 (secreted glycoprotein)(AA 1-256)) (Cat #ABIN2000167) from Sino Biological (https://www.antibodies-online.com); FSTL3 antibody (AA 145-159) from antibodies-online (Cat #ABIN2621090) from antibodies online (https://www.antibodies-online.com); Mouse Follistatin-related Gene Protein/FLRG Antibody (Cat #AF1255) from R&D Systems (https://www.rndsystems.com); activin A recombinant human protein (Cat #PHC9564) from Thermo Fisher Scientific (https://www.thermofisher.com); recombinant human activin B protein (Cat #Q53T31), Bone Morphogenic Protein 6 (Cat #P22004), and Bone Morphogenic Protein 7 (Cat #P18075) from R&D Systems (https://www.rndsystems.com); myostatin human from Sigma-Aldrich (Cat #MFCD03457656, https://www.sigmaaldrich.com).

Some of MMP7 binding elements. In some embodiments, the binding element comprises an element selected from: SOMAmer SL000525 from SomaLogic Inc (somalogic.com/); MMP-7 Polyclonal Antibody (Cat #bs-0423R) from Bioss (https://www.biossusa.com/); MMP7 antibody—C-terminal region (Cat #ARP46075_T100) from Aviva Systems Biology (https://www.avivasysbio.com/); MMP7 antibody (Matrix Metallopeptidase 7 (Matrilysin, Uterine)) (C-Term) (Cat #ABIN321032) from antibodies-online (https://www.antibodies-online.com/).

Some of GDF15 binding elements. In some embodiments, the binding element comprises an element selected from: SOMAmer SL003869 from SomaLogic Inc (somalogic.com/); Growth Differentiation Factor 15 (GDF15) Antibody (Cat #abx210159) from Abbexa (https://www.abbexa.com/); GDF15 antibody (Growth Differentiation Factor 15) (C-Term) (Cat #ABIN453961) from antibodies-online (https://www.antibodies-online.com/); Mouse GDF-15 Antibody (Cat #AF6385) from R&D Systems (https://www.rndsystems.com); Monoclonal antibodies to growth and differentiation factor 15 (gdf-15) from WO 2014049087A1 (https://www.google.com/patents/WO2014049087A1?cl=en); anti-MIC-1 antibodies from WO 2009021293A1 (https://www.google.com/patents/WO2009021293A1?cl=en); Recombinant Human GFR alpha-like His-tag Protein (GFRAL) (Cat #9647-GR-050) from R&D Systems (https://www.rndsystems.com); Recombinant Human CLPTM1 protein (Cat #ab158143) from Abcam (http://www.abcam.com/); Recombinant Human QRFPR, His-tagged (Cat #QRFPR-2093H) from Creative BioMart (https://www.creativebiomart.net/); any of gdf-15 antibodies described in application WO2014100689A1; any of gdf-15 antibodies described in application WO2015144855A1, any of gdf-15 antibodies described in application Application US20180142013A1, any of gdf-15 antibodies described in application US20170306008A1, any of the agents described in application KR20180030898A, WO2017189724A1, U.S. Pat. No. 9,175,076B2, US20170137506A1, AV-380 (a humanized Immunoglobulin G 1 (IgG1) inhibitory monoclonal antibody) and other anti-GDF15 antibodies, licensed by AVEO Oncology (NASDAQ:AVEO) to Novartis.

Some of FRZB binding elements. In some embodiments, the binding element comprises an element selected from: SOMAmer SL004650 from SomaLogic Inc (somalogic.com/); Human/Mouse sFRP-3 Antibody (Cat #MAB1921) from R&D Systems (https://www.rndsystems.com/); FRZB antibody (Frizzled-Related Protein) (Middle Region)(Cat #ABIN2785778) from Aviva Systems Biology (https://www.antibodies-online.com/); FRZB antibody (Frizzled-Related Protein) (Cat #ABIN610965) from antibodies-online (https://www.antibodies-online.com/); FRZB antibody (Frizzled-Related Protein) (Cat #ABIN958985) from antibodies-online (https://www.antibodies-online.com/); Anti-FRZB Mouse mAb (1H8) (Cat #ST1697) from Merck (http://www.merckmillipore.com/); Anti-FRZB Rabbit pAb (Cat #ST1696) from Merck (http://www.merckmillipore.com/); EGF Recombinant Human Protein (Cat #PHG0311) from Thermo Fisher Scientific (http://www.thermofisher.com/); Recombinant Wnt-1 protein (Cat #PHC1804) from Thermo Fisher Scientific (http://www.thermofisher.com/); Recombinant Human Wnt-3a Protein (Cat #5036-WN) from R&D Systems (https://www.rndsystems.com/); Recombinant Human Wnt5a protein (Cat #ab204627) from Abcam (http://www.abcam.com/).

Some of TNFRSF1B binding elements. In some embodiments, the binding element comprises an element selected from: SOMAmer SL001800 from SomaLogic Inc (somalogic.com/); TNFRSF1B antibody (Cat #ABIN1105812) from antibodies-online (https://www.antibodies-online.com/); Mouse TNF RII/TNFRSF1B Antibody (Cat #AF-426-PB) from R&D Systems (https://www.rndsystems.com); Recombinant Human TNF-alpha Protein (Cat #210-TA) from R&D Systems (https://www.rndsystems.com); Recombinant Human Progranulin Protein (PGRN) (Cat #2420-PG) from R&D Systems (https://www.rndsystems.com); Transmembrane TNF (tmTNF); TNF-SAM2; Atsttrin; and Membrane bound trimeric TNFα (mTNFa 3)

Some of CD55 binding elements. In some embodiments, the binding element comprises an element selected from: SOMAmer SL004556 from SomaLogic Inc (somalogic.com/); CD55 antibody—middle region (ARP60155_P050) (Cat #ARP60155_P050) from Aviva Systems Biology (https://www.avivasysbio.com/); Mouse CD55/DAF Antibody (Cat #MAB5376) from R&D Systems (https://www.rndsystems.com/); CD55 antibody (Complement Decay-Accelerating Factor) (Cat #ABIN212850) from antibodies-online (https://www.antibodies-online.com/); CD55 (143-30) antibody (Cat #mab71260) from Covalab (https://www.covalab.com/); Monoclonal Antibody to Decay Accelerating Factor (DAF) (Cat #MAA468Hu22) and Polyclonal Antibody to Decay Accelerating Factor (DAF) (Cat #PAA468Hu0) from Cloud-Clone (http://www.cloud-clone.com/); CD55 antibody (Complement Decay-Accelerating Factor) (Domain SCR4) (Cat #ABIN94183), CD55 antibody (Complement Decay-Accelerating Factor) (Cat #ABIN2192138) and CD55 antibody (Complement Decay-Accelerating Factor) (Cat #ABIN291344) from antibodies-online (https://www.antibodies-online.com/); CD97 Protein, Human, Recombinant (His Tag) (Cat #11280-H08H) from Sino Biological (http://www.sinobiological.com/); SNV (SIn Nombre Hantavirus); CB3 (coxsackievirus B3); AfaE-III; SNV (Sin Nombre virus); hantaviruses; C3b/Factor B/Bb; and echovirus 11; PB102 and PB108 (anti-CD55 chimeric monoclonal antibodies) of Percipio Biotherapeutics, Inc.

Some of RGMB binding elements. In some embodiments, the binding element comprises an element selected from: SOMAmer SL010468 from SomaLogic Inc (somalogic.com/); RGMB antibody (RGM Domain Family, Member B) (Center) (Cat #ABIN2706945) from Cohesion Biosciences (https://www.antibodies-online.com/); Mouse RGM-B Antibody (Cat #AF3597) from R&D Systems (https://www.rndsystems.com/); PD-L2 Protein, Human, Recombinant (His Tag) (Cat #10292-H08H) from Sino Biological (http://www.sinobiological.com/); Recombinant Human E Cadherin protein (Cat #ab38254) from Abcam (http://www.abcam.com/); Neogenin (Netrin receptor) (Cat #NBP1-89653PEP) from Novus Biologicals (https://www.novusbio.com/); Recombinant Human BMP-4 Protein (Cat #314-BP) and Recombinant Human/Mouse/Rat BMP-2 Protein (CAt #355-BM) from R&D Systems (https://www.rndsystems.com/); IL6 Recombinant Human Protein (Cat #PHC0066) from Thermo Fisher Scientific (http://www.thermofisher.com/).

Some of FAS binding elements. In some embodiments, the binding element comprises an element selected from: SOMAmer SL002731 from SomaLogic Inc (somalogic.com/); Purified Mouse Anti-CD95 Clone 13/Fas (RUO) (Cat #610197) from BD Biosciences (https://www.bdbiosciences.com/); Mouse Fas/TNFRSF6/CD95 Antibody (Cat #AF435) from R&D Systems (https://www.rndsystems.com/); CD95/Fas Antibody (Cat #AHP2306) from Bio-Rad (https://www.bio-rad-antibodies.com/). In some embodiments, the binding element (anti-FAS) comprises an antibody contained in medications or developing drug candidates selected from: APO010 mega FasLigandof Apoxis S.A., Bio-Link, Oncology Venture, Topotarget A S (Intravenous, APO010 is a synthetic, recombinant, soluble, hexameric fusion protein consisting of three human Fas ligand (FasL) extracellular domains fused to the dimer-forming collagen domain of human adiponectin with potential pro-apoptotic and antineoplastic activities. Assembled into a soluble hexameric structure mimics the ligand clustering of endogenous active FasL, Fas receptor agonist APOO10 activates the Fas receptor, resulting in caspase-dependent apoptosis in susceptible tumor cell populations); DE098 of Centocor, Inc., Argenes Inc, Santen Pharmaceutical Co Ltd (other names) ARG 098, ARG098, DE 098; F45D9 (mAb F45D9) of IMED AB company; F61F12 (mAb F61F12) of IMED AB company; Fsn1103 of Fusion Therapeutics; Myocardial Infarction Therapy SERVIER of CNRS—Centre national de la recherche scientifique, Les Laboratoires Servier; Novotarg of Celonic GmbH, Baliopharm A G Novotarg is a bispecific antibody targeting cluster of differentiation 20 (CD20) and cluster of differentiation 95 (CD95). Novotarg acts as an agonist to CD95, which is a death receptor and mediates apoptosis in the cells expressing CD20 and their structural analogs.

Some of NBL1 binding elements. In some embodiments, the binding element comprises an element selected from: SOMAmer SL005156 from SomaLogic Inc (somalogic.com/); NBL1 (Middle Region) antibody (Cat #ARP56637_P050) from Aviva Systems Biology (https://www.avivasysbio.com/); Mouse DAN Antibody (Cat #AF755) from R&D Biosystems (https://www.rndsystems.com/); Neuroblastoma 1, DAN Family BMP Antagonist (NBL1) (AA 21-32) antibody (Cat #ABIN1916174) from antibodies-online (https://www.antibodies-online.com/); Recombinant Human/Mouse/Rat BMP-2 Protein (CAt #355-BM), Recombinant Human BMP-4 Protein (Cat #314-BP), Recombinant Human BMP-7 Protein (Cat #354-BP), and Recombinant Human P-Selectin/CD62P Protein (Cat #ADP3-200) from R&D Systems (https://www.rndsystems.com/);

Some of CCD80 binding elements. In some embodiments, the binding element comprises an element selected from: SOMAmer SL010390 from SomaLogic Inc (somalogic.com/); CCDC80 Antibody (Cat #ARP66333_P050) from Aviva Systems Biology (https://www.avivasysbio.com/); Polyclonal Antibody to Coiled Coil Domain Containing Protein 80 (CCDC80) (Cat #PAJ901Hu02) and Coiled-Coil Domain Containing 80 (CCD80) (AA 82-288) antibody (Cat #PAJ901Hu01) from Cloud-Clone (https://www.cloud-clone.us/); Coiled-Coil Domain Containing 80 (CCD80) (AA 721-770) antibody (Cat #ABIN1834250) from antibodies-online (https://www.antibodies-online.com/); Recombinant human JAK2 protein (Cat #ab42619) from Abcam (http://www.abcam.com/); and PKGIa.

Some of PTN binding elements. In some embodiments, the binding element comprises an element selected from: SOMAmer SL002704 from SomaLogic Inc (somalogic.com/); PTN monoclonal antibody (M01), clone 5C3 (AA 45-154) (Cat #H00005764-M01) from Abnova (http://www.abnova.com/); Pleiotrophin antibody (PTN) (C-Term) (Cat #ABIN2782191) from Aviva Systems Biology (https://www.antibodies-online.com/); Pleiotrophin antibody (PTN) (AA 141-155) (Cat #ABIN793112) from antibodies-online (https://www.antibodies-online.com/); anaplastic lymphoma kinase (ALK) Recombinant Human Protein (Cat #PV3867) from Thermo Fisher Scientific (http://www.thermofisher.com/); Recombinant Human Syndecan-1 protein (Cat #ab191664) from Abcam (http://www.abcam.com/); Syndecan-3 Protein Human Recombinant (ECD, His Tag) (Cat #12158-H08H) from Sino Biological (http://www.sinobiological.com/); Recombinant Human Syndecan-2/CD362 Protein (Cat #2965-SD) from R&D Systems (https://www.rndsystems.com/); Dermatan sulfate (Cat #C3788) from Sigma Aldrich (https://www.sigmaaldrich.com/); Chondroitin Sulfate (Cat #C6737) from Sigma Aldrich (https://www.sigmaaldrich.com/); Recombinant Human Neuroglycan C/CSPG5 Protein (Cat #5685-NG) from R&D Systems (https://www.rndsystems.com/); protein tyrosine phosphatase receptor PTPC; 6B4 proteoglycan; oligosaccharides isolated from E-CS/DS.

Some of CD59 binding elements. In some embodiments, the binding element comprises an element selected from: SOMAmer SL00455 from SomaLogic Inc (somalogic.com/); CD59 antibody (CD59) (Cat #NB500-330) from Novus Biologicals (https://www.novusbio.com/); CD59 antibody (CD59) (Cat #ABIN400702) from antibodies-online (https://www.antibodies-online.com/); CD59 antibody (CD59) (AA 1-101) (Cat #12474-H08H) from Sino Biological (https://www.sinobiological.com/); Monoclonal Antibody to Protectin (CD59) (Cat #MAB336Hu22) from Cloud-Clone (https://www.cloud-clone.us/); CD59 antibody (CD59) (Cat #ABIN94204) from antibodies-online (https://www.antibodies-online.com/); CD59 antibody (CD59) (AA 25-104) (Cat #ABIN5662056) from Cloud-Clone (https://www.antibodies-online.com/); CD59 antibody (CD59) (AA 23-105) (Cat #ABIN1174501) from Cloud-Clone (https://www.antibodies-online.com/); CD59 antibody (CD59) (AA 21-107) (Cat #ABIN1174507) form Cloud-Clone (https://www.antibodies-online.com/); CD59 antibody (CD59) (AA 26-98) (Cat #ABIN1858309) from Cloud-Clone (https://www.antibodies-online.com/); CD59 Monoclonal Antibody (YTH53.1) (Cat #MA1-81489) from Thermo Scientific (https://www.thermofisher.com/); Transmembrane protein Tmp21 (035587); Transmembrane emp24 domain-containing protein 2 (Q15363); Transmembrane emp24 domain-containing protein 10 (Q28735); cholesterol-dependent cytolysin (CDC) family of pore-forming toxins (PFTs); CD2 (P06729); C9H petide (GSFRFSYSKNETYQL); sp22 peptide (ACHWPWCHGWHSACDLPMHPMC); C8a MACPF protein domain; Fragment of Streptococcus intermedius toxin (Intermedilysin, ILY).

Some of NTN1 binding elements. In some embodiments, the binding element comprises an element selected from: SOMAmer SL01239 from SomaLogic Inc (somalogic.com/); Netrin-1 Antibody (Nora-1) (Cat #NBP1-97641) from Novus Biologicals (https://www.novusbio.com/); Netrin 1 antibody (NTN1) (Cat #ABIN265184), Netrin 1 antibody (NTN1) (AA 23-449) (Cat #ABIN2581529), Netrin 1 antibody (NTN1) (AA 498-509) (Cat #ABIN609671), Netrin 1 antibody (NTN1) (Cat #ABIN212236), Netrin 1 antibody (NTN1) (AA 32-51) (Cat #ABIN2626387), Netrin 1 antibody (NTN1) (AA 591-604) (Cat #ABIN769046) from antibodies-online (https://www.antibodies-online.com/); Death-associated protein kinase 1(P53355); DCC (P43146); NEO1 (Q92859); UNC5C (095185); UNC5B (Q81ZJ1); A3β1 integrin; Deleted in Colorectal Carcinomas (DCC) (P43146); Down's syndrome Cell Adhesion Molecule (DSCAM) (060469); glycosaminoglycans; DCC-5Fbn; β-amyloid precursor protein; CD146 (P43121).

Some of POSTN binding elements. In some embodiments, the binding element comprises an element selected from: SOMAmer SL005084 from SomaLogic Inc (somalogic.com/); Periostin(POSTN) Mouse Monoclonal Antibody (Cat #TA804575) from OriGene (https://www.origene.com/); POSTN antibody (Periostin, Osteoblast Specific Factor) (AA 251-300) (Cat #ABIN958318) and POSTN antibody (Periostin, Osteoblast Specific Factor) (AA 811-824) (Cat #ABIN2627028) from antibodies-online (https://www.antibodies-online.com/); integrin αvβ3/MPC5B4; benzyl-d(U)TP-modified DNA aptamers (doi: 10.1038/mt.2013.30); Antiperiostin-F(ab′)2 (doi:10.2967/jnumed.115.156216); metalloproteinases (MMPs); mAb OC-20 (doi: 10.1016/j.ejca.2011.04.026); Recombinant Bone morphogenetic protein 1 (Cat #RPA653Hu01) from Cloud-Clone (http://www.cloud-clone.com/).

Some of MMP7 binding elements. In some embodiments, the binding element comprises an element selected from: SOMAmer SL005213 from SomaLogic Inc (somalogic.com/); RELT rabbit monoclonal antibody (Cat #H00084957-K) from Abnova (http://www.abnova.com/); Rabbit polyclonal antibody to RELT (Cat #CPA2825) from Cohesion Biosciences (http://www.cohesionbio.com/); HVR-H1 sequence of SEQ ID NO: 49, HVR-H2 sequence of SEQ ID NO: 58, and HVR-H3 sequence of SEQ ID NO: 67 (US20080057066A1); Recombinant serine/threonine-protein kinase OSR1 protein (Cat #10727-H09B) from Sino Biological (http://www.sinobiological.com/).

Some of EFEMP1 binding elements. In some embodiments, the binding element comprises an element selected from: SOMAmer SL006527 from SomaLogic Inc (somalogic.com/); EFEMP1 Antibody (Cat #OAPB00902) from Aviva Systems Biology (https://www.avivasysbio.com/); Fibulin 3 (FBLN3) (AA 396-445) antibody (Cat #ABIN2620917), Fibulin 3 (FBLN3) (C-Term) antibody (Cat #ABIN468726), and Fibulin 3 (FBLN3) (AA 25-42) antibody (Cat #ABIN329130) from antibodies-online (https://www.antibodies-online.com/); Recombinant Human MMP-2 Protein (Cat #902-MP), Recombinant Human DLL3 Protein (Cat #9749-DL), Recombinant Human EGFR Protein (Cat #095-ER), and Recombinant Human ECM-1 Protein (Cat #3937-EC) from R&D Systems (https://www.rndsystems.com/); Recombinant Human TGF beta Receptor I protein (Cat #ab70837) from Abcam (http://www.abcam.com/); Short consensus repeat region (SCR7) (doi:10.1159/000431141).

Some of GAS1 binding elements. In some embodiments, the binding element comprises an element selected from: SOMAmer SL000658 from SomaLogic Inc (somalogic.com/); GAS1 antibody (Growth Arrest-Specific 1) (AA 102-136) (Cat #ABIN952461) from antibodies-online (https://www.antibodies-online.com/); Recombinant Human Sonic Hedgehog (Shh) (Cat #100-45) from PeproTech (https://www.peprotech.com/); extracellular part of RET; Hh (Hedgehog) proteins.

Some of KLK11 binding elements. In some embodiments, the binding element comprises an element selected from: SOMAmer SL002763 from SomaLogic Inc (somalogic.com/); Monoclonal Antibody to Kallikrein 11 (KLK11) (Cat #MAA669Hu22) from Cloud-Clone (https://www.cloud-clone.us/); KLK11 Polyclonal Antibody (Cat #E-AB-11357) from Elabscience (https://www.elabscience.com/); Kallikrein 11 antibody (KLK11) (Center) (Cat #ABIN2855789) and Kallikrein 11 antibody (KLK11) (AA 233-250) (Cat #ABIN2624570) from antibodies-online (https://www.antibodies-online.com/); HIP binding elements (http://www.xray.cz/setkani/abst2014/mareckova.htm).

Some of UNC5C binding elements. In some embodiments, the binding element comprises an element selected from: SOMAmer SL005230 from SomaLogic Inc (somalogic.com/); Unc5c antibody (Unc-5 Homolog C (C. Elegans)) (AA 812-823) (Cat #ABIN1496204) and Unc5c antibody (Unc-5 Homolog C (C. Elegans)) (AA 395-444) (Cat #ABIN575028) from antibodies-online (https://www.antibodies-online.com/); UNC5C Antibody (N-term) (Cat #AP19910a) from Abgent (http://www.abgent.com/); UNC5C Antibody—middle region (Cat #OAAB04034) from Aviva Systems Biology (https://www.avivasysbio.com/); Anti-UNC5C antibody produced in rabbit (Cat #HPA012086) from Sigma-Aldrich (https://www.sigmaaldrich.com/); Anti-UNC5C antibody (Cat #ab106949) from Abcam (http://www.abcam.com/); Recombinant Human FLRT2 Protein (Cat #NBP2-52291) from Novus Biologicals (https://www.novusbio.com/); Recombinant Human Netrin-1 Protein (Cat #6419-N1) and Recombinant Human DSCAM Protein (Cat #3666-DS) from R&D Systems (https://www.rndsystems.com); Recombinant human Fyn protein (Cat #Ab84696) from Abcam (www.abcam.com).

Some of B2M binding elements. In some embodiments, the binding element comprises an element selected from: SOMAmer SL000283 from SomaLogic Inc (somalogic.com/); Beta2-Microglobulin (12B2) Antibody (Cat #250426) from Abbiotec (https://www.abbiotec.com/); Beta-2 Microglobulin Antibody [clone SPM374] (Cat #V2799) from NSJ Bioreagents (https://www.nsjbio.com/); Anti-Beta-2 microglobulin Antibody (Cat #80423-R026) from Sino Biological (https://www.sinobiological.com/); beta-2 Microglobulin antibody (beta-2-Microglobulin) (APC) (Cat #ABIN4284017) from Novus Biologicals (https://www.novusbio.com/); beta-2 Microglobulin antibody (beta-2-Microglobulin) (Cat #ABIN236862) and beta-2 Microglobulin antibody (beta-2-Microglobulin) (AA 22-34) (Cat #ABIN1100157) from antibodies-online (https://www.antibodies-online.com/); single-chain variable region (scFv) antibody fragment (https://www.sciencedirect.com/science/article/pii/S0085253815497054); Recombinant Human LILRBi/CD85j/ILT2 Fc Chimera Protein (Cat #2017-T2) and Recombinant Human LILRB3/CD85a/ILT5 Fc Chimera Protein (Cat #1806-T5) from R&D Systems (https://www.rndsystems.com/); CD8+T lymphocytes (https://www.ncbi.nlm.nih.gov/pubmed/12096037); HLA-B27 (http://www.jimmunol.org/content/jimmunol/early/2012/05/16/jimmunol.1102711.full.pdf); M. tuberculosis EsxA (ESAT-6) and an EsxA-EsxB (CFP-10) complex (http://www.cansar.icr.ac.uk/cansar/molecular-targets/P61769/).

In some embodiments, the binding element (for B2M) comprises a molecule selected from the following:

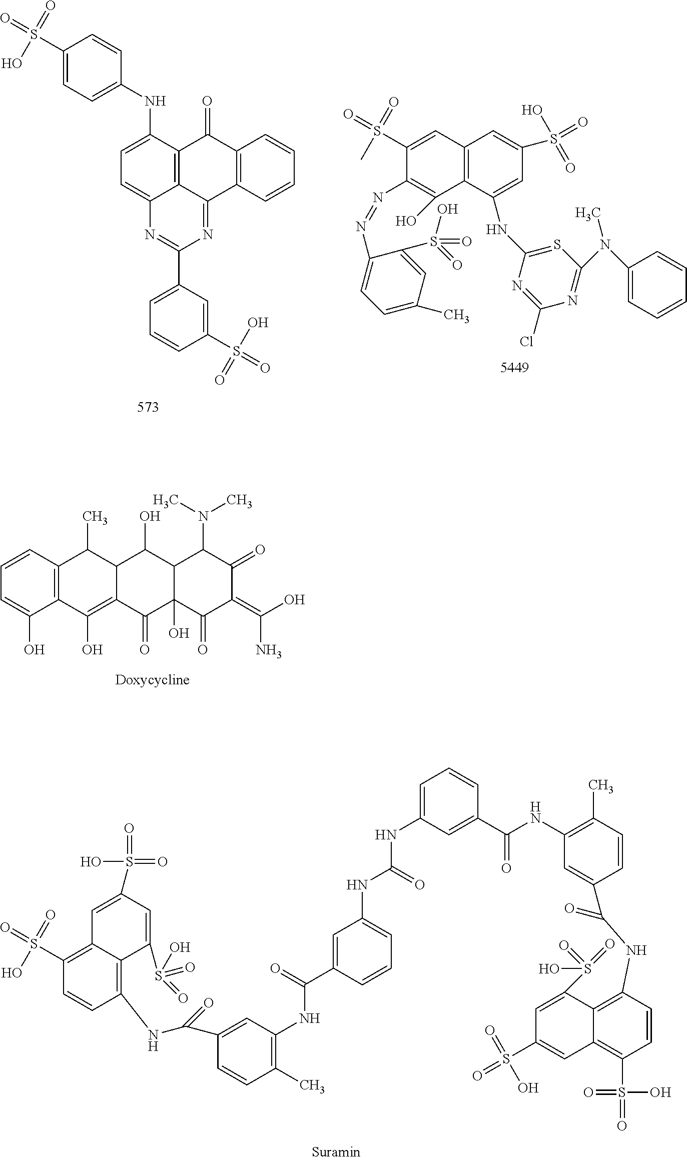

Some of TNFRSF1A binding elements. In some embodiments, the binding element comprises an element selected from: SOMAmer SL005230 from SomaLogic Inc (www.somalogic.com/); Anti-TNFRSF1A antibody (Cat #STJ72751) from St John's Laboratory (www.stjohnslabs.com/); TNFRSF1A antibody (Tumor Necrosis Factor Receptor Superfamily, Member 1A) (Extracellular Domain) (Cat #ABIN799835), TNFRSF1A antibody (Tumor Necrosis Factor Receptor Superfamily, Member 1A) (Cat #ABIN462093), TNFRSF1A antibody (Tumor Necrosis Factor Receptor Superfamily, Member 1A) (AA 195-211) (Cat #ABIN1105799) from antibodies-online (https://www.antibodies-online.com/); Recombinant Human TNF-alpha Protein (Cat #ab9642) from Abcam (www.abcam.com).

Some of COL18A1 binding elements. In some embodiments, the binding element comprises an element selected from: SOMAmer SL000403 from SomaLogic Inc (www.somalogic.com/); Endostatin Antibody (Cat #AF1098) from Novus Biologicals (https://www.novusbio.com); Rabbit Polyclonal COL18A1 antibody (Cat #abx008024) and COL18A1 antibody (Collagen, Type XVIII, alpha 1) (Cat #abx001439) from Abbexa (https://www.abbexa.com/).

Some of DPT binding elements. In some embodiments, the binding element comprises an element selected from: SOMAmer SL008178 from SomaLogic Inc (somalogic.com/); DPT Antibody (N-term) (Cat #AP12641a) from Abgent (http://www.abgent.com/); DPT Antibody—middle region (Cat #OAAB04540) from Aviva Systems Biology (https://www.avivasysbio.com/); Dermatopontin antibody (DPT) (AA 45-94) (Cat #ABIN798671) and Dermatopontin antibody (DPT) (AA 45-94) (Cat #ABIN798671) from antibodies-online (https://www.antibodies-online.com/); Monoclonal Antibody to Dermatopontin (DPT) (Cat #MAC432Hu22) from Cloud-Clone (http://www.cloud-clone.com/); Dermatopontin Antibody (Cat #NB110-68135) from Novus Biologicals (https://www.novusbio.com/); Decorin/Bone proteoglycan II human (Cat #SRP6454) from Sigma-Aldrich (http://www.uniprot.org/uniprot/P21793); Recombinant human TGF beta 1 protein (Cat #ab50036) from Abcam (http://www.abcam.com/).

Some of MMP7 binding elements. In some embodiments, the binding element comprises an element selected from: SOMAmer SL003770 from SomaLogic Inc (somalogic.com/); SFRP1 antibody (Secreted Frizzled-Related Protein 1) (AA 171-183) (Cat #ABIN574064), SFRP1 antibody (Secreted Frizzled-Related Protein 1) (Middle Region) (Cat #ABIN926020), SFRP1 antibody (Secreted Frizzled-Related Protein 1) (Middle Region) (Cat #ABIN2792235), and SFRP1 antibody (Secreted Frizzled-Related Protein 1) (AA 26-55) (Cat #ABIN652980) from antibodies-online (https://www.antibodies-online.com/); Recombinant Human Myocilin protein (Cat #ab151657) from Abcam (http://www.abcam.com/); WNT1 Recombinant Human Protein (Cat #PHC1804) from ThermoFischer Scientific (http://www.thermofisher.com/); Recombinant Human/Mouse Wnt-5a Protein (Cat #645-WN) from R&D Systems (https://www.rndsystems.com/); Recombinant Human Thrombospondin-1 Protein (Cat #3074-TH) from R&D Systems (https://www.rndsystems.com/); L/V-VDGRW-L/V peptide (doi:10.1016/j.peptides.2004.07.010).

Some of SMOC1 binding elements. In some embodiments, the binding element comprises an element selected from: SOMAmer SL011888 from SomaLogic Inc (somalogic.com/); SMOC1 antibody (SPARC Related Modular Calcium Binding 1) (AA 150-221) (Cat #ABIN566231) from antibodies-online (https://www.antibodies-online.com/); Recombinant Human Tenascin C Protein (Cat #3358-TC) from R&D Systems (https://www.rndsystems.com/); Recombinant Human Pro-epidermal growth factor protein(EGF) (Cat #CSB-AP002581HU) from Cusabio (https://www.cusabio.com/); heparin and heparan sulfate.

Some of STC1 binding elements. In some embodiments, the binding element comprises an element selected from: SOMAmer SL005789 from SomaLogic Inc (somalogic.com/); Stanniocalcin 1 antibody (STC1) (AA 150-167) (Cat #ABIN2628872) and Stanniocalcin 1 antibody (STC1) (N-Term) (Cat #ABIN320700) from antibodies-online (https://www.antibodies-online.com/); Monoclonal Antibody to Stanniocalcin 1 (STC1) (Cat #MAC874Hu22) from Cloud-Clone (https://www.cloud-clone.us/); Recombinant Human PAPP A protein (Cat #ab53389) from Abcam (http://www.abcam.com/).

Some of BMP4 binding elements. In some embodiments, the binding element comprises an element selected from: BMP4 antibody (Bone Morphogenetic Protein 4) (Cat #ABIN364236) and BMP4 antibody (Bone Morphogenetic Protein 4) (AA 264-313) (Cat #ABIN798412) from antibodies-online (https://www.antibodies-online.com/); BMP4 Antibody (Cat #71-071) from ProSci (https://www.prosci-inc.com/); Monoclonal Antibody to Bone Morphogenetic Protein 4 (BMP4) (Cat #MAA014Hu22) from Cloud-Clone (http://www.cloud-clone.com/); Recombinant Human Agrin Protein (Cat #8909-AG) from R&D Systems (https://www.rndsystems.com); Recombinant Human BMPER protein (Cat #ab165805), Recombinant Human Fibrillin 1 protein (Cat #ab114345), and Recombinant Human Fibrillin 2 protein (Cat #ab114937) from Abcam (www.abcam.com); BMPR2 Recombinant Human Protein (Cat #PV6256) from ThermoFisher Scientific (http://www.thermofisher.com/); Recombinant Sclerostin Domain Containing Protein 1 (SOSTDC1) (Cat #RPF900Mu01) and Recombinant Low Density Lipoprotein Receptor Related Protein 2 (LRP2) (Cat #RPD101Hu01) from Cloud-Clone (http://www.cloud-clone.com/).

Some of s-FRP-1 binding elements. In some embodiments, the binding element comprises an element selected from: diphenylsulfone-sulfonamide s-FRP-1 inhibitor (CAS 915754-88-0) (Cat #344300) from Merckmillipore (http://www.merckmillipore.com/); iminothiazolidinone derivatives, including but not limited to from US20060270720A1 (https://patents.google.com/patent/US20060270720); WAY 316606 hydrochloride (Cat #4767) from Tocris (https://www.tocris.com/); piperidinyl diphenylsulfonyl sulfonamides.

An additional non-liniting list of binding elements is shown in Example 1.

In some embodiments, the binding element comprises an aptamer. In some embodiments, the binding element comprises a DNA aptamer. In some embodiments, the binding element comprises a RNA aptamer. In some embodiments, the binding element comprises a XNA aptamer. In some embodiments, the binding element comprises a chemically modified RNA or DNA aptamer or oligonucleotide. In some embodiments, the binding element comprises a peptide aptamer. In some embodiments, the binding element comprises an affimer. In some embodiments, the binding element comprises SOMAmer.

In some embodiments, the binding element comprises a polypeptide. In some embodiments, the GDF15 binding element, comprises polypeptide which comprises following sequence selected from

[SEQ ID NO: 28]
GSIYIHVYFTKSGFHPDPRQG;
[SEQ ID NO: 29]
KALYRRLATVHMSRMINKYKG;
[SEQ ID NO: 30]
ITINIVDDHTPWVKGSVPPPG;
[SEQ ID NO: 31]
LDQYVKFDAVSGDYYPIIYFG;
[SEQ ID NO: 32]
YPINESLASLPLRVSFCPLSG;
[SEQ ID NO: 33]
GDYYPIIYFNDYWNLQKDYYG;
[SEQ ID NO: 34]
GSIYIHVYFTKSGFHPDPRQ;
[SEQ ID NO: 35]
KALYRRLATVHMSRMINKYK;
[SEQ ID NO: 36]
ITINIVDDHTPWVKGSVPPP;
[SEQ ID NO: 37]
LDQYVKFDAVSGDYYPIIYF;
[SEQ ID NO: 38]
YPINESLASLPLRVSFCPLS;
and
[SEQ ID NO: 39]
GDYYPIIYFNDYWNLQKDYY

and other agents from patent application WO2017013188A1 incorporated here by reference, including but not limited to (Formula I), (Formula II), SEQ ID from NO. 1 to NO. 322.

In some embodiments, the binding element comprises a virus. In some embodiments, the binding element comprises a helical virus. In some embodiments, the binding element comprises an icosahedral virus.

In some embodiments, a binding element comprises a ligand of a cell surface receptor. In some embodiments, the ligand is a naturally-occurring ligand or a synthetic ligand. In some embodiments, the ligand is a native ligand of the receptor (e.g., a ligand that is produced by a subject in vivo) or a non-native ligand (e.g., a ligand that is introduced into the subject, such as a virus or drug). In some embodiments, the binding element comprises a ligand for a cytosolic receptor or a nuclear receptor.

In some embodiments, the binding element comprises a small molecule. In some embodiments, the binding element comprises a drug or other molecule. In some embodiments the such drug or other molecule is known to selectively bind, modify or degrade a target protein.

In some embodiments, the MMP7 binding element comprises one or more of a small molecules listed below:

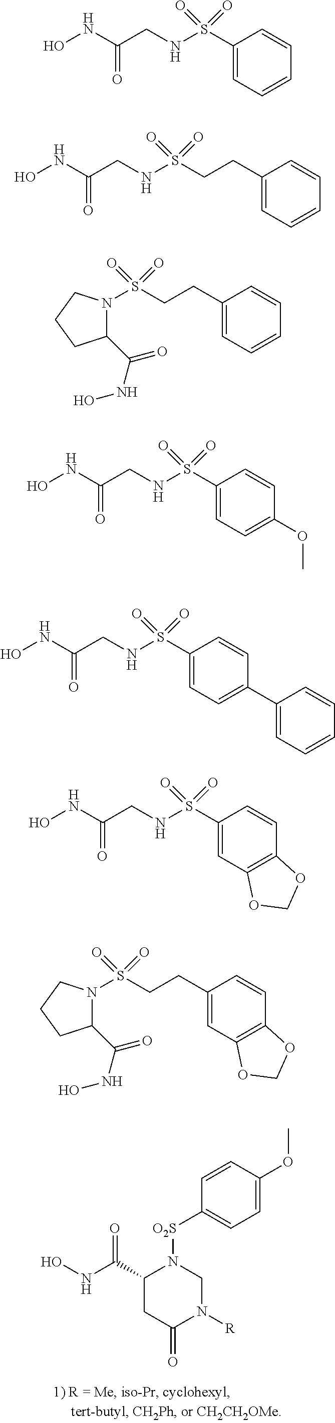

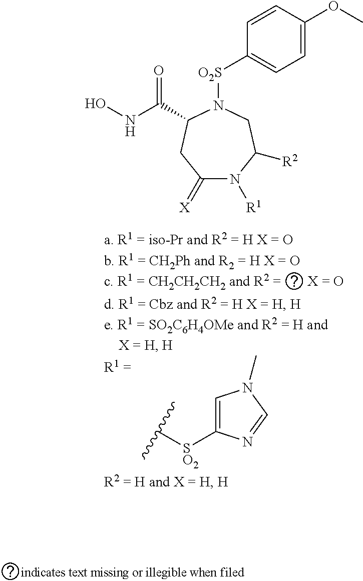

In some embodiments, the binding element comprises a molecular sieve.

In some embodiments, the lumen within the housing comprises a binding element of a known amount. In some embodiments, more than 5 pmol of binding element is present. In some embodiments, more than 10 pmol of binding element is present. In some embodiments, more than 20 pmol of binding element is present. In some embodiments, more than 50 pmol of binding element is present. In some embodiments, more than 100 pmol of binding element is present. In some embodiments, more than 1 nmol of binding element is present. In some embodiments, more than 5 nmol of binding element is present. In some embodiments, more than 10 nmol of binding element is present. In some embodiments, more than 20 nmol of binding element is present. In some embodiments, more than 50 nmol of binding element is present. In some embodiments, more than 100 nmol of binding element is present. In some embodiments, more than 0.3 μmol of binding element is present. In some embodiments, more than 0.5 μmol of binding element is present. In some embodiments, more than 1 μmol of binding element is present. In some embodiments, more than 5 μmol of binding element is present. In some embodiments, more than 10 pmol of binding element is present. In some embodiments, more than 20 μmol of binding element is present. In some embodiments, more than 100 μmol of binding element is present. In some embodiments, from about 1 pmol to about 100 μmol of binding element is present. In some embodiments, from about 5 pmol to about 50 μmol of binding element is present. In some embodiments, from about 50 pmol to about 10 μmol of binding element is present. In some embodiments, from about 100 pmol to about 1 μmol of binding element is present. In some embodiments, from about 500 pmol to about 1 μmol of binding element is present. In some embodiments, from about 1 nmol to about 100 μmol of binding element is present. In some embodiments, from about 1 nmol to about 1 μmol of binding element is present. In some embodiments, from about 1 nmol to about 500 nmol of binding element is present. In some embodiments, from about 5 pmol to about 100 nmol of binding element is present. In some embodiments, from about 10 pmol to about 50 nmol of binding element is present.

In some embodiments, the binding element is immobilized in the lumen of the housing. In some embodiments, the binding element is bound through a covalent bond. In some embodiments, the binding element is bound through and ionic bond. In some embodiments, the binding element is bound through an electrostatic interaction. In some embodiments, the binding element is attached to the platform by use of click chemistry.

In some embodiments, bonding occurs through reactive substituent groups on the binding element such as primary amines, sulfhydryls, aldehydes, and carboxylic acids. The stationary phase may be first activated with a compound that is reactive toward one or more of these functional groups. The activated complex may generate a covalent linkage between the binding element and the stationary phase, resulting in binding element immobilization. In some embodiments, the binding element is attached to the platform or stationary phase by usage the interaction between biotin and streptavidin

In general, the stationary phase is a material that is insoluble in water buffers, plasma, or other hydrophilic solutions. In preferred embodiments, the stationary phase is selected from biocompatible materials to prevent denaturation and other negative impacts to desirable plasma elements, i.e., molecules in plasma that are not being selectively removed. In some embodiments, the stationary phase comprises granules. In some embodiments, the stationary phase comprises plastic spheres. In some embodiments, the stationary phase comprises a solid matrix. In some embodiments, the stationary phase comprises a resin, e.g., an affinity resin such as an ion exchange resin. In some embodiments, the stationary phase comprises sepharose, e.g., a sepharose bead. In some embodiments, the stationary phase is CNBr-activated sepharose. In some embodiments, the stationary phase is Carboxylated-functionalized sepharose 4FF beads. In some embodiments, the stationary phase is epoxy-activated sepharose. In some embodiments, the stationary phase is azide-functionalized sepharose. In some embodiments, the stationary phase comprises a gel matrix. In some embodiments, the stationary phase comprises an adsorption gel. In some embodiments, the gel matrix is a viscous material. In some embodiments, the gel matrix is a gel bead with an average diameter between 0.1-10 mm. In a preferred embodiments, stationary phase is sorbent.

In some embodiments, the gel matrix is suspended in a solution, e.g., a solution buffered to physiological conditions, and the suspension is fills the lumen of the housing. The lumen may contain a glass filter near the inlet port and a glass filter near the outlet port, such that plasma can pass through the filters while the gel matrix cannot. In some embodiments, the gel matrix comprises a sepharose gel. In some embodiments, the gel matrix comprises an agarose gel.

In some embodiments of the device, the inlet port of the housing is configured to receive plasma from a whole blood separator. For example, the inlet port may be in fluid communication, with a whole blood separator, e.g., through tubing, such that plasma separated in the whole blood separator, flows into the inlet of the device. In some embodiments of the device, the whole blood separator comprises a centrifuge. In some embodiments, the device is configured to be operably linked to a pump for advancing fluid into the inlet port of the housing. For example, a pump may provide force to drive plasma into the inlet of the housing of the device and/or force plasma through the stationary phase in the housing. In some embodiments, the device is configured for use without a pump. In some embodiments, the device is configured for gravitational flow of plasma through the housing. In some embodiments, the device further comprises a first filter element positioned to filter plasma released from the housing. In some embodiments, the first filter element is situated in the outlet port. In some embodiments, a second filter element positioned to filter fluid entering the housing. In some embodiments, the second filter element is situated in the inlet port.

In certain embodiments, the disclosure provides a protein binding device comprising a platform associated with a first group of binding elements and a second group of binding elements, wherein each of the first group and second group of binding elements selectively bind a protein selected from the group: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB, wherein the first group of binding elements are selective for a different protein than the second group of binding elements. In some embodiments, the first group and second group of binding elements are covalently or electrostatically attached to the platform. In some embodiments, the first group and second group of binding elements are covalently attached to the platform. In some embodiments, the first group and second group of binding elements electrostatically attached to the platform. In some embodiments, the first group and second group of binding elements ionically bonded to the platform. In some embodiments, the first group and second group of binding elements covalently bonded to the platform. In certain embodiments, the disclosure provides a protein binding device capable of selective binding of more than two proteins selected from the group: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB.

Accordingly, in such embodiments, this disclosure provides a protein binding device comprising a platform associated with a first group of binding elements and a second group of binding elements, and a third group of binding elements and any further groups of binding elements, wherein each of the first group and second and third and any further group of binding elements selectively bind a protein selected from the group: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB, wherein the first group of binding elements are selective for a different protein than the second group of binding elements and a third group of binding elements and each further group of binding elements.

In some embodiments, the protein binding device is a diagnostic chip. In some embodiments, the platform comprises a rigid material. In some embodiments, the platform comprises of flexible material. In some embodiments, the platform comprises a plastic, e.g., polystyrene.

In some embodiments of the device, each of the first group of binding elements and the second group of binding elements are selected from antibodies, proteins, polymers, aptamers, peptides, viruses, and small molecules. In some embodiments of the device, the first group of binding elements and the second group of binding elements are selected from antibodies. In some embodiments of the device, the first group of binding elements and the second group of binding elements are selected monoclonal antibodies and polyclonal antibodies. In some embodiments of the device, the first group of binding elements and the second group of binding elements are selected from humanized monoclonal antibodies and humanized polyclonal antibodies.

In some embodiments, the first binding elements selectively bind the protein with a K_{D} of less than 1×10⁻⁶ M. In some embodiments, the first binding elements selectively bind the protein with a K_{D} of less than 1×10⁻⁷ M. In some embodiments, the first binding elements selectively bind the protein with a K_{D} of less than 1×10⁻⁸ M. In some embodiments, the first binding elements selectively bind the protein with a K_{D} of less than 1×10⁻⁹ M. In some embodiments, the first binding elements selectively bind the protein with a K_{D} of less than 1×10⁻¹² M. In some embodiments, the second binding elements selectively bind the protein with a K_{D} of less than 1×10⁻⁶ M. In some embodiments, the second binding elements selectively bind the protein with a K_{D} of less than 1×10⁻⁷ M. In some embodiments, the second binding elements selectively bind the protein with a K_{D} of less than 1×10⁻⁸ M. In some embodiments, the second binding elements selectively bind the protein with a K_{D} of less than 1×10⁻⁹ M. In some embodiments, the second binding elements selectively bind the protein with a K_{D} of less than 1×10⁻¹² M. In certain embodiments, the first binding element selectively binds the protein with a K_{D} of about 1×10⁻³M to about 1×10⁻¹⁵M. In certain embodiments, the first binding element selectively binds the protein with a K_{D} of about 1×10⁻³M to about 1×10⁻¹⁴M. In certain embodiments, the first binding element selectively binds the protein with a K_{D} of about 1×10⁻³M to about 1×10⁻¹³M. In certain embodiments, the first binding element selectively binds the protein with a K_{D} of about 1×10⁻³M to about 1×10⁻¹²M. In certain embodiments, the first binding element selectively binds the protein with a K_{D} of about 1×10⁻³M to about 1×10⁻¹¹M. In certain embodiments, the first binding element selectively binds the protein with a K_{D} of about 1×10⁻³M to about 1×10⁻¹⁰M. In certain embodiments, the first binding element selectively binds the protein with a K_{D} of about 1×10⁻³M to about 1×10⁻⁹M. In certain embodiments, the first binding element selectively binds the protein with a K_{D} of about 1×10⁻³M to about 1×10⁻⁸M. In certain embodiments, the first binding element selectively binds the protein with a K_{D} of about 1×10⁻³M to about 1×10⁻⁷M. In certain embodiments, the first binding element selectively binds the protein with a K_{D} of about 1×10⁻³M to about 1×10⁻⁶M. In certain embodiments, the first binding element selectively binds the protein with a K_{D} of about 1×10⁻⁶M to about 1×10⁻¹⁵M. In certain embodiments, the first binding element selectively binds the protein with a K_{D} of about 1×10⁻⁹M to about 1×10⁻¹⁵M. In certain embodiments, the first binding element selectively binds the protein with a K_{D} of about 1×10⁻¹²M to about 1×10⁻¹⁵M. In certain embodiments, the first binding element selectively binds the protein with a K_{D} of about 1×10⁻⁶M to about 1×10⁻¹²M. In certain embodiments, the first binding element selectively binds the protein with a K_{D} of about 1×10⁻⁶M to about 1×10⁻⁹M. In certain embodiments, the second binding element selectively binds the protein with a K_{D} of about 1×10⁻³M to about 1×10⁻¹⁵M. In certain embodiments, the second binding element selectively binds the protein with a K_{D} of about 1×10⁻³M to about 1×10⁻¹⁴M. In certain embodiments, the second binding element selectively binds the protein with a K_{D} of about 1×10⁻³M to about 1×10⁻¹³M. In certain embodiments, the second binding element selectively binds the protein with a K_{D} of about 1×10⁻³M to about 1×10⁻¹²M. In certain embodiments, the second binding element selectively binds the protein with a K_{D} of about 1×10⁻³M to about 1×10⁻¹¹M. In certain embodiments, the second binding element selectively binds the protein with a K_{D} of about 1×10⁻³M to about 1×10⁻¹⁰M. In certain embodiments, the second binding element selectively binds the protein with a K_{D} of about 1×10⁻³M to about 1×10⁻⁹M. In certain embodiments, the second binding element selectively binds the protein with a K_{D} of about 1×10⁻³M to about 1×10⁻⁸M. In certain embodiments, the second binding element selectively binds the protein with a K_{D} of about 1×10⁻³M to about 1×10⁻⁷M. In certain embodiments, the second binding element selectively binds the protein with a K_{D} of about 1×10⁻³M to about 1×10⁻⁶M. In certain embodiments, the second binding element selectively binds the protein with a K_{D} of about 1×10⁻⁶M to about 1×10⁻¹⁵M. In certain embodiments, the second binding element selectively binds the protein with a K_{D} of about 1×10⁻⁹M to about 1×10⁻¹⁵M. In certain embodiments, the second binding element selectively binds the protein with a K_{D} of about 1×10⁻¹²M to about 1×10⁻¹⁵M. In certain embodiments, the second binding element selectively binds the protein with a K_{D} of about 1×10⁻⁶M to about 1×10⁻¹²M. In certain embodiments, the second binding element selectively binds the protein with a K_{D} of about 1×10⁻⁶M to about 1×10⁻⁹M.

In some embodiments, the binding element comprises a molecularly imprinted polymer having a crosslinked core, wherein the core has molecular sized cavities adapted to selectively receive and bind target molecules. In some embodiments, such target molecules are selected from Targets.

In some embodiments, the lumen within the housing comprises a molecularly imprinted polymer having a crosslinked core, wherein the core has molecular sized cavities adapted to selectively receive and bind target molecules. In some embodiments, such target molecules are one or more proteins in plasma selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB. One of the examples of syntheses of molecularly imprinted polymer is shown in Example 15 Plasma Filtration

In certain embodiments, the disclosure provides a plasma filtration device, wherein the plasma filtration device comprises a housing defining a lumen and a filter element configured to contact and filter plasma introduced to the lumen of the housing, wherein the filter element selectively captures one or more proteins in plasma selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB. In certain embodiments, the disclosure provides a fluid filtration device, wherein the fluid filtration device comprises a housing defining a lumen and a filter element configured to contact and filter fluid introduced to the lumen of the housing, wherein the filter element selectively captures a protein in fluid selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB, wherein the fluid is optionally plasma.

In some embodiments, the fluid filtration device comprises a housing with at least one inlet port and at least one outlet port. In some embodiments, the fluid filtration device is suited to operably couple to a fluid source. In some embodiments, the fluid filtration device is operably coupled to a fluid source at the inlet port. In some embodiments, the fluid filtration device is configured such that fluid can pass through the inlet port into the device, undergo filtration within the lumen, and be extruded through the outlet port. In some embodiments, the fluid is plasma. In some embodiments, the outlet port is in fluid communication with a lumen containing plasma-deficient blood, wherein plasma from the outlet port is able to combine with the plasma-deficient blood to create whole blood.

In some embodiments the plasma source is a whole blood fractionator. In some embodiments, the blood fractionator separates blood fractions through centrifugation.

In some embodiments, the plasma filtration device comprises a filter element within the housing that captures a select protein or proteins by molecular size exclusion. In some embodiments the device is configured to be operably coupled to a component that generates an electromagnetic field or electromagnetic gradient to advance the plasma through the filter element. In some embodiments, the device is configured to be operably coupled to a pump to advance the plasma through the filter element.

Pharmaceutical Compositions of Protein Binding Elements

In certain embodiments, the disclosure provides a pharmaceutical composition comprising a binding element that binds to, modulates, or degrades a protein, e.g., a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB; and at least one pharmaceutically acceptable excipient. In some embodiments, the binding element inhibits or degrades the protein. In some embodiments, the binding element is the active agent of a pharmaceutical formulation. In some embodiments, the binding element binds to, modulates, or degrades a secondary protein which has an upstream or downstream effect on a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB. In some embodiments, the binding element is unbound, e.g., unbound from a particle. Some non-limiting examples of pharmaceutical compositions, comprising binding elements are shown in Examples 31 and Example 32.

In some embodiments, the pharmaceutical composition, comprising an agent modulating or binding or inhibiting or degrading or activating at least one effector upstream or downstream of at least one of the Targets can activate or modulate in any other way something (protein, another body element etc) upstream of the Target that will reduce Target protein indirectly or inhibit Target indirectly its activity or in some other similar way cause the same effect as reduction of Target protein. In some embodiments, the device, comprising a binding element at least one effector upstream of at least one of the Targets can reduce in any other way something (protein, another body element etc) upstream of the Target that will reduce Target protein indirectly or inhibit Target indirectly its activity or in some other similar way cause the same effect as reduction of Target protein.

In some embodiments, the pharmaceutical composition, comprising an agent modulating or binding or inhibiting or degrading or activating at least one effector upstream or downstream of at least one of the Targets can intervene downstream of the Target protein, e.g. but not limited to if reducing Target cause increase of some protein (e.g. protein X) downstream of Target, adding of such protein X into the bloodstream will mimic Target reduction while the actual level of Target will be the same. In this case the medication comprising protein X is encompassed by this invention. Another example, if reducing Target protein causes also reduction of some protein Y, than reducing such protein Y (method of treatment, method of reduction) are also encompassed by this invention, as well as devices, pharmaceutical compositions for such reduction and treatment.

In some embodiments, a binding element is bound, e.g., covalently bound, to a particle. In certain embodiments, the pharmaceutical composition is administered to a subject in need thereof, e.g., parenterally administered, and the protein binding element binds to, modulates, or degrades a protein. In some embodiments, the binding element binds target proteins and removes the target proteins from the body, e.g., upon excretion of the particle from the body.

In some embodiments, a binding element is reversibly bound to a particle, and can be released into a body following administration. The binding element binds the target protein and the complex is removed from the body via physiological processes.

Particles

In some embodiments, the term “particle” refers to a small mass that can comprise any material, such as alumina, metal (e.g., gold or platinum), glass, silica, latex, plastic, agarose, polyacrylamide, methacrylate or any polymeric material, and be of any size and shape. In some embodiments, the particle or particles comprise silicon. (See, e.g., International Patent Application Publication Nos. WO 2013/011764, WO 2013/029278, and WO 2014/151381, and U.S. Patent Application Publication No. 2014/0271886, the disclosures of each of which are incorporated by reference in their entirety). In some embodiments, the particles comprise starch (see, e.g., International Patent Application Publication No. WO 2010/084088). In some embodiments, the particle or particles comprise nucleic acid (e.g., naturally-occurring or non-naturally occurring nucleic acid). Methods for making such nucleic acid-based microscopic structures are known in the art and are described in, e.g., Douglas et al., Nuc Acids Res 37(15):5001-5006 (2009); Douglas et al., Nature 459(7245):414-428 (2009); Voigt et al., Nat Nanotechnol 5(3):200-203 (2010); and Endo et al., Curr Protoc Nucleic Acid Chem Chapter 12(Unit 12.8) (2011).

In some embodiments, a particle is insoluble in aqueous solution {e.g., the particle may be insoluble in water, blood serum, blood plasma, extracellular fluid, and/or interstitial fluid). For example, in some embodiments, a particle is separated from aqueous solution by centrifuging a solution comprising the particle, e.g., at speeds that are sufficient to separate the cells of a cell suspension from the aqueous solution of the cell suspension.

In some embodiments, a particle readily exists as a suspension in aqueous solution, e.g., mild shaking or vortexing of a plurality of particles in aqueous solution is sufficient to suspend the particles in the solution. In some embodiments, the particle is not a hydrogel. In some embodiments, the particle does not comprise a hydrogel. In some embodiments, the particle does not comprise a polymer.

A particle is preferably large enough to bind to more than one protein and inhibit the interaction of more than one bound protein with a binding partner. For example, in some embodiments, a particle is about 50 nm to about 10 μm. In some embodiments, a particle is 1 μm to 5 μm in size, 1.2 μm to 4 μm, 1.5 μm to 4 μm, or 2 μm to 4 μm.

In some embodiments, particles with sizes less than 300 nm, such as less than 200 nm or less than 150 nm, are preferred for applications in which the particles are intended to enter and/or exit the vasculature of a subject, such as particles that are administered by subcutaneous injection. In some embodiments, larger particles are similarly well-suited for subcutaneous injection for methods in which the particles are not intended to enter the vasculature. In some embodiments, particles with sizes of about 1 μm to about 5 μm are preferable for applications in which the particles are intended to circulate within the vasculature of a subject, e.g., following intravenous administration. In some embodiments, particles with sizes greater than 5 μm are preferable for applications in which the particles are intended to reside at the site in which they are implanted, such as within or adjacent to a tumor; however, in some embodiments, particles smaller than 5 μm are also suitable for implantation. In some embodiments, particles of any size are utilized for in vitro applications.

Also featured herein are collections of particles. In some embodiments, a plurality of particles has a narrow or broad polydispersity. As used herein, “polydispersity” refers to the range of sizes of particles within a particular particle population. That is, an extremely polydisperse population might involve particles having a mean size of, say, 1 μm with individual particles ranging from 0.1 to 4 μm. In some embodiments, a “narrow polydispersity” is preferred. That is, given a particular mean particle size, it is presently preferred that individual particles in the population differ by no more than ±20%, preferably no more than ±15%, and most preferably at present no more than ±10% from the mean particle size. More specifically, a particle population preferably has a mean particle size of about 0.5 to about 2 μm, more preferably at present from about 0.8 to about 1.5 μm. Thus, if a mean particle size of 1 μm is selected, individual particles in the population would most preferably be within the range of from about 0.8 to about 1.2 μm. In some embodiments, the particle population has a mean particle size of about 0.3 to about 1 μm, e.g., about 0.4 to about 0.9, about 0.5 to about 0.9, about 0.4 to about 0.8, about 0.5 to about 0.7, about 0.3 to about 0.9, or about 0.3 to about 0.7 μm. In some embodiments, the particle population has a mean particle size of about 1 μm to about 10 μm, e.g., about 1.1 μm to about 4.8, about 1.2 μm to about 4.6, about 1.4 μm to about 4.4, about 1.6 μm to about 4.2, about 1.8 μm to about 4.0, or about 2.0 μm to about 3.8 μm.

In some embodiments, the disclosure features a collection or plurality of particles having a defined mean particle size. As used herein, “mean particle size” is arrived at by measuring the size of individual particles and then dividing by the total number of particles. The determination of mean particle size is well known in the art. Typically, the longest average dimension of the particles is no greater than 4 μm. In some embodiments, the longest average dimension of the particles is no greater than 3.9 (e.g., no greater than 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, or 1) μm. In some embodiments, the longest average dimension of the particles is no greater than 2.5 μm, 2 μm, 1.5 μm, or 1.25 μm. In some embodiments, the longest average dimension of the particles is at least 1 μm, but no greater than 4 μm. In some embodiments, the longest average dimension of the particles is at least 1 μm, but no greater than 2 μm. In some embodiments, the longest average dimension of the particles is at least 1 μm, but no greater than 1.5 μm. In some embodiments, the longest average dimension of the particles is at least 0.5 m\(e.g., at least 0.6, 0.7, 0.8, 0.9, 1, 1.1, 1.2, 1.3, 1.4, or 1.5 μm), but no greater than 4 μm (e.g., no greater than 3.9 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2, 1.9, 1.8, 1.7, or 1.6 μm).

In some embodiments, the particles are nanoparticles. In some embodiments, the longest average dimension of the particles is no greater than 900 nm (e.g., 850, 800, 750, 700, 650, 600, 550, 500, 450, 400, 450, 400, 350, 300, 250, 200, or 150 nm). In some embodiments, a particle is shaped and sized to circulate in the blood or vasculature (e.g., arteries, veins, and capillaries) of a subject (e.g., a human subject).

In some embodiments, the longest dimension of the particle is about 50 nm to about 5 μm, such as about 100 nm to about 4.5 μm, about 200 nm to about 4 μm, about 300 nm to about 3.5 μm, about 300 nm to about μm, or about 400 nm to about 3 μm. In some embodiments, the shortest dimension of the particle is at least about 300 nm, such as about 300 nm to about 4 μm or about 400 nm to about 3 μm.

In some embodiments, a plurality of the particles are polyhedral, e.g., cubic. In some embodiments, a plurality of the particles are spherical. In some embodiments, any of the particles described herein can be porous. Such porous particles comprise an outer surface and inner surfaces of the pores of the particle. The binding element can be, e.g., bound to the inner surfaces. In some embodiments, a plurality of pores have a cross-sectional dimension of at least 50 nm. In some embodiments, a plurality of pores have a cross-sectional dimension of at least 100 nm. Porous nanoparticles have been described in, e.g., U.S. Patent Application Publication Nos. 20140199352, 20080277346, and 20040105821, the disclosures of each of which are incorporated by reference in their entirety. Spherical particles are described in, e.g., U.S. Pat. Nos. 8,778,830 and 8,586,096, each of which is hereby incorporated by reference.

In some embodiments, spherical particles can further comprise two intersecting ridges extending from the spherical surface of the particle, wherein the longest dimension of each of the structures is no greater than 4 μm (e.g., no greater than 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, or 1 μm). In some embodiments, a plurality of particles are toroidal. In such embodiments, the binding element can be bound to an inner circumferential surface of the particle. In some embodiments, the diameter of the particle is no greater than 4 μm (e.g., 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, or 1 μm). In some embodiments, the diameter of the particle is no greater than 900 nm (e.g., 850, 800, 750, 700, 650, 600, 550, 500, 450, 400, 350, 300, 200, or 150 nm).

In some embodiments, a plurality of particles are polyhedral, e.g., octahedral or icosahedral, whether regular or irregular. In some embodiments, the particles comprise at least one protrusion from at least one of their vertices. In some embodiments, the particles comprise more than one (e.g., 2, 3, 4, 5, 6, 7, or 8 or more) protrusion from their vertices.

In some embodiments, a particle comprises void space, referred to as a “void” or “voids” herein. A void is the space in a particle that is filled by a fluid (e.g., a liquid, which may comprise a protein or a gas, such as when a particle is dried) or by empty space (e.g., when a particle is in a vacuum, such as after lyophilization). In some embodiments, the void volume of a particle includes, for example, the pore volume of a particle and/or the volume of the interior of a hollow core/shell particle, the lumen of a tube, torus, or ring.

In some embodiments, a particle is configured such that blood plasma may freely enter and/or exit the void space of the particle, e.g., when the particle is located in the vasculature of a subject. In some embodiments, a particle is configured such that blood serum may freely enter and/or exit the void space of the particle, e.g., when the particle is located in the vasculature of a subject. In preferred embodiments, a particle is configured such that blood cells cannot enter the void space of the particle. In some embodiments, a particle is configured such that platelets cannot enter the void space of the particle.

Nevertheless, a particle may allow for a platelet to enter its void space, e.g., when the particle is configured for use in vitro or when the particle is configured to bind a virus, bacterium, protein, fungal or yeast cell, or other large target, such as a target sized from about 100 nm to about 2 μm.

In some embodiments, a particle is configured such that extracellular fluid may freely enter and/or exit the void space of the particle. In some embodiments, a particle is configured such that interstitial fluid may freely enter and/or exit the void space of the particle. In some embodiments, a particle is configured such that cerebrospinal fluid may freely enter and/or exit the void space of the particle.

The volume of the void space in a particle is preferentially large enough to accommodate more than one protein. Nevertheless, a void may be smaller than the total volume of each bound protein so long as the particle is capable of inhibiting interactions between each bound protein and the second members of the binding pairs that include each protein. For example, a particle may need only sequester a binding site of a protein to inhibit interactions between the protein and a second member of a binding pair, and such a particle may contain a void volume that accommodates the binding site of each protein but that allows for other portions of one or more proteins to project outward from the void space.

In some embodiments, a particle may comprise about 5% to about 95% void space. A particle comprising protrusions may comprise little or no void space, e.g., because the protrusions may inhibit interactions between a bound protein and a second member of a binding pair. A particle comprising a tube may comprise a large amount of void space, e.g., because a tube may comprise a large internal volume relative to the thickness of the walls of the tube. Nevertheless, the void volume of particles with similar geometries may comprise varying amounts of void volume, e.g., tubes comprising walls of the same thickness may vary substantially in void volume percentage depending on tube diameter.

In some embodiments, a particle may comprise 0% to about 40% void space, about 20% to about 60% void space, about 40% to about 80% void space, or about 60% to 100% void space. A particle may comprise 0% to about 20% void space, about 10% to about 30% void space, about 20%) to about 40% void space, about 30% to about 50% void space, about 40% to about 60% void space, about 50% to about 70% void space, about 60%> to about 80%>void space, about 70% to about 90% void space, or about 80% to 100% void space. In some embodiments, a particle may comprise 0% to about 10% void space, about 5% to about 15% void space, about 10% to about 20%) void space, about 15% to about 25% void space, about 10% to about 20% void space, about 15% to about 25% void space, about 10% to about 20% void space, about 15%) to about 25% o void space, about 10% to about 20% void space, about 15% to about 25% o void space, about 20% to about 30% void space, about 25% to about 35% void space, about 30%) to about 40% void space, about 35% to about 45% void space, about 40% to about 50%) void space, about 45% to about 55% void space, about 50% to about 60% void space, about 55% to about 65% void space, about 60% to about 70% void space, about 65% to about 75%) void space, about 70% to about 80% void space, about 75% to about 85% void space, about 80% to about 90% void space, about 85% to about 95% void space, or about 90% to 100% void space.

In some embodiments, a particle comprises a neutral charge at physiological pH (e.g., −7.4). In some embodiments, a particle comprises a slightly negative or slightly positive charge at physiological pH. The surface of a particle (e.g., outer surface) may comprise a slightly negative or slightly positive charge at physiological pH. In preferred embodiments, the surface of a particle (e.g., outer surface) comprises a slightly negative or neutral charge at physiological pH. The isoelectric point of the particle may be about 5 to about 9, preferably about 6 to about 8. Particles comprising a nucleic acid may have an isoelectric point of about 4 to about 7. In some embodiments, the isoelectric point of the particle is less than 7.4, i.e., such that the particle has a net negative charge at physiological pH. For example, the isoelectric point of the particle may be about 6.0 to about 7.4, such as about 6.4 to about 7.4. A particle comprising a net negative charge at physiological pH is less likely to interact with eukaryotic cells (e.g., mammalian cells) because eukaryotic cells generally comprise cell membranes with a net negative charge. A particle preferably does not comprise sufficient charge (and/or charge density) to engage in non-specific interactions with other charged molecules.

Particles Comprising Pores

In some embodiments, the material used to make the particles (e.g., silicon) may have a porosity of about 40% to about 95%, such as about 60% to about 80%. Porosity, as used herein, is a measure of the void spaces in a material, and is a fraction of the volume of voids over the total volume of the material. In certain embodiments, the carrier material has a porosity of at least about 10%, at least about 20%, at least about 30%>, at least about 40%), at least about 50%, at least about 60%>, at least about 70%, at least about 80%>, or even at least about 90%. In particular embodiments, the porosity is greater than about 40%, such as greater than about 50%, greater than about 60%, or even greater than about 70%.

In certain embodiments, the binding element is distributed to a pore depth from the surface of the material of at least about 0.005 μm, at least 0.05 μm, at least about 0.1 μm, at least about 0.2 μm, at least about 0.3 μm, at least about 0.4 μm, at least about 0.5 μm, at least about 0.6 μm, or at least about 0.7 μm. In certain embodiments, the binding element is distributed in the pores of the carrier material substantially uniformly.

In some embodiments, the binding element may be loaded into the particle to a depth which is measured as a ratio to the total width of the particle. In certain embodiments, the binding element is distributed to a depth of at least about 10% into the particle, to at least about 20% into the particle, at least about 30%) into the particle, at least about 40% into the particle, at least about 50% into the particle, or at least about 60% into the particle.

In some embodiments, the pore size may be preselected to the dimensional characteristics of the binding element and target protein to control the binding of the protein. Typically, pore sizes that are too small preclude loading of the binding element and/or binding of the protein. For example, the average pore diameter for a material may be selected from larger pores, e.g., 15 nm to 40 nm, for high molecular weight molecules, e.g., 200,000-500,000 amu, and smaller pores, e.g., 2 nm to 10 nm, for molecules of a lower molecular weight, e.g., 10,000-50,0000 amu. For instance, average pore sizes of about 6 nm in diameter may be suitable for molecules of molecular weight around 14,000 to 15,000 amu such as about 14,700 amu. Average pore sizes of about 10 nm in diameter may be selected for molecules of molecular weight around 45,000 to 50,000 amu such as about 48,000 amu. Average pore sizes of about 25-30 nm in diameter may be selected for molecules of molecular weight around 150,000 nm. The pore size may be preselected to be adapted to the molecular radii of the binding element or protein. For instance, average pore sizes of about 25 nm to about 40 nm in diameter may be suitable for molecules with a largest molecular radius from about 6 nm to about 8 nm. Molecular radii may be calculated by any suitable method such as by using the physical dimensions of the molecule based on the X-ray crystallography data or using the hydrodynamic radius which represents the solution state size of the molecule.

Particles Comprising at Least One Tube

In some embodiments, the particle comprises at least one tube. In preferred embodiments, the at least one tube comprises one open end or two open ends.

In some embodiments, the term “tube” refers to a three-dimensional shape having a length along an axis (e-g-, a one-dimensional axis in Cartesian space) and an internal cavity, lumen, void, or reservoir along the length of the shape. In some embodiments, perpendicular cross sections along the axis of the tube have a substantially identical shape and/or size. The term “cross section,” as used in relation to a tube, refers to a two-dimensional cross section that is perpendicular to the axis of the tube. A larger structure may comprise a tube. For example, a syringe comprises a tube, but the tube does not comprise the syringe plunger. A particle or other article may comprise more than one tube. For example, a syringe may comprise two tubes corresponding to the syringe needle and the syringe barrel, or to parallel barrels of a double syringe (e.g., used for epoxy compositions). A tube may have a diameter, which is the average length of the line segments that are perpendicular to the axis of the tube, wherein each line segment is bounded by two points on the outer surface of the tube. A tube may have a width and height, wherein the width of the tube is the longest line segment defined by two points on the outer surface of the tube that is perpendicular to the axis of the tube, and the height of the tube is the line segment defined by two points on the outer surface of the tube that is perpendicular to both the axis of the tube and the line segment defining the width of the tube.

In some embodiments, a tube may have an internal diameter, which is the average length of the line segments that are perpendicular to the axis of the tube, wherein each line segment is bounded by two points on the inner surface of the tube. A tube may have an internal width and internal height, wherein the internal width of the tube is the longest line segment defined by two points on the outer surface of the tube that is perpendicular to the axis of the tube, and the internal height of the tube is the line segment defined by two points on the outer surface of the tube that is perpendicular to both the axis of the tube and the line segment defining the width of the tube.

In some embodiments, a tube may be substantially cylindrical. The tube may have a substantially circular cross section. The cross section of the tube may be an ellipsoid, such as a circle.

In some embodiments, the cross section of the tube may be a polygon, such as a regular polygon. The cross section of the tube may be a triangle, such as an equilateral triangle. The cross section of the tube may be a quadrilateral, such as a regular quadrilateral, a rectangle, or a square. The cross section of the tube may be a pentagon, such as a regular pentagon. The cross section of the tube may be a hexagon, such as a regular hexagon. A tube may be a triangular tube, square tube, pentagonal tube, hexagonal tube, heptagonal tube, or octahedral tube.

In some embodiments, the length of a tube may be about 5 nm to about 5 μm, such as about 5 nm to about 4 μm, about 5 nm to about 3 μm, about 5 nm to about 2 μm, or about 5 nm to about 1 μm. The length of a tube may be about 50 nm to about 5 μm, such as about 50 nm to about 4 μm, about 50 nm to about 3 μm, about 50 nm to about 2 μm, or about 50 nm to about 1 μm. The length of a tube may be about 100 nm to about 5 μm, such as about 100 nm to about 4 μm, about 100 nm to about 3 μm, about 100 nm to about 2 μm, or about 100 nm to about 1 μm. The length of a tube may be about 300 nm to about 5 μm, such as about 300 nm to about 4 μm, about 300 nm to about 3 μm, about 300 nm to about 2 μm, or about 300 nm to about 1 μm. The length of a tube may be about 500 nm to about 5 μm, such as about 500 nm to about 4 μm, about 500 nm to about 3 μm, about 500 nm to about 2 μm, or about 500 nm to about 1 μm.

In some embodiments, the diameter, width, and/or height of a tube may be about 5 nm to about 5 μm, such as about 5 nm to about 4 μm, about 5 nm to about 3 μm, about 5 nm to about 2 μm, about 5 nm to about 1 μm, about 5 nm to about 900 nm, about 5 nm to about 800 nm, about 5 nm to about 700 nm, about 5 nm to about 600 nm, about 5 nm to about 500 nm, about 5 nm to about 400 nm, about 5 nm to about 300 nm, about 5 nm to about 200 nm, or about 5 nm to about 100 nm. The diameter, width, and/or height of a tube may be about 50 nm to about 5 μm, such as about 50 nm to about 4 μm, about 50 nm to about 3 μm, about 50 nm to about 2 μm, about 50 nm to about 1 μm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 200 nm, or about 50 nm to about 100 nm.

In some embodiments, the internal diameter, internal width, and/or internal height of a tube are preferentially large enough to accommodate both the binding element and the target protein. The internal diameter, internal width, and/or internal height of a tube are preferentially small enough to inhibit a cell from entering the interior of the tube (e.g., a nucleated eukaryotic cell, such as a nucleated human cell or a diploid human cell). The internal diameter, internal width, and/or internal height of a tube may be about 5 nm to about 4 μm, such as about 5 nm to about 3 μm, about 5 nm to about 2 μm, about 5 nm to about 1 μm, about 5 nm to about 900 nm, about 5 nm to about 800 nm, about 5 nm to about 700 nm, about 5 nm to about 600 nm, about 5 nm to about 500 nm, about 5 nm to about 400 nm, about 5 nm to about 300 nm, about 5 nm to about 200 nm, or about 5 nm to about 100 nm. The internal diameter, internal width, and/or internal height of a tube may be about 20 nm to about 4 μm, such as about 20 nm to about 3 μm, about 20 nm to about 2 μm, about 20 nm to about 1 μm, about 20 nm to about 900 nm, about 20 nm to about 800 nm, about 20 nm to about 700 nm, about 20 nm to about 600 nm, about 20 nm to about 500 nm, about 20 nm to about 400 nm, about 20 nm to about 300 nm, about 20 nm to about 200 nm, or about 20 nm to about 100 nm. The internal diameter, internal width, and/or internal height of a tube may be about 40 nm to about 4 μm, such as about 40 nm to about 3 μm, about 40 nm to about 2 μm, about 40 nm to about 1 μm, about 40 nm to about 900 nm, about 40 nm to about 800 nm, about 40 nm to about 700 nm, about 40 nm to about 600 nm, about 40 nm to about 500 nm, about 40 nm to about 400 nm, about 40 nm to about 300 nm, about 40 nm to about 200 nm, or about 40 nm to about 100 nm.

In certain preferred embodiments, the particle comprises a plurality of tubes. Each tube of the plurality of tubes may be substantially parallel. In some embodiments, at least two tubes of the plurality of tubes are not parallel. In some embodiments, none of the tubes of the plurality of tubes are parallel. The tubes may be arranged in a configuration other than parallel to distribute the openings to the tubes over different faces of the particle or to allow the particle to tumble in flow (e.g., laminar flow or turbulent flow).

In some embodiments, a plurality of tubes may be arranged in a lattice or bundle. In some embodiments, a plurality of tubes may be arranged in a polyhedron, such as a regular polyhedron. In some embodiments, the plurality of tubes may be arranged in a tetrahedron, such as a regular tetrahedron. The plurality of tubes may be arranged in a hexahedron, such as a cuboid, rectangular cuboid, or cube. The plurality of tubes may be arranged in an octahedron, such as a regular octahedron. The plurality of tubes may be arranged in a dodecahedron, such as a regular dodecahedron. The plurality of tubes may be arranged in an icosahedron, such as a regular icosahedron. In some embodiments, each edge of the polyhedron is defined by a single tube. In some embodiments, less than each edge of the polyhedron is defined by a single tube (e.g., when each of the tubes are substantially parallel). In some embodiments, a plurality of tubes may be arranged in a pyramid, such as a triangular pyramid, rhombic pyramid, rectangular pyramid, square pyramid, pentagonal pyramid, hexagonal pyramid, heptagonal pyramid, or octagonal pyramid. The plurality of tubes may be arranged in a right pyramid or an oblique pyramid. In some embodiments, each edge of the pyramid is defined by a single tube. In some embodiments, less than each edge of the pyramid is defined by a single tube (e.g., when each of the tubes are substantially parallel). In some embodiments, a plurality of tubes may be arranged in a prism, such as a triangular prism, rectangular prism, square prism, pentagonal prism, hexagonal prism, heptagonal prism, or octagonal prism. The plurality of tubes may be arranged in a right prism, an oblique prism, or a truncated prism. In some embodiments, each edge of the prism is defined by a single tube. In some embodiments, less than each edge of the prism is defined by a single tube (e.g., when each of the tubes are substantially parallel).

In some embodiments, a plurality of tubes may be arranged in a configuration that has a length, width, and height, wherein no single dimension is more than 5 times larger than any other dimension. For example, the plurality of tubes may be arranged in a configuration wherein no single dimension is more than 4 times larger than any other dimension or no single dimension is more than 3 times larger than any other dimension. Such configurations are favorable, for example, for intravenous administration of a particle because oblong particles may not flow as well in a patient's bloodstream.

In some embodiments, a plurality of tubes may be arranged in a configuration that has a length and diameter, wherein the length of the configuration is not more than 5 times its diameter. For the plurality of tubes may be arranged in a configuration wherein the length of the configuration is not more than 4 times its diameter or the length of the configuration is not more than 3 times its diameter. Such configurations are favorable, for example, for intravenous administration of the particle because oblong particles may not flow as well in a patient's bloodstream.

In some embodiments, a particle may comprise 1 to 500 tubes, such as 1 to 100 tubes. A particle may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 330, 31, 32, 33, 34, 35, 36, 37, 38, 39, 40, 41, 42, 43, 44, 45, 46, 47, 48, 49, 50, 51, 52, 53, 54, 55, 56, 57, 58, 59, 50, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 tubes.

In some embodiments, a plurality of tubes may comprise 1 to 500 tubes, such as 1 to 100 tubes. A plurality of tubes may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 330, 31, 32, 33, 34, 35, 36, 37, 38, 39, 40, 41, 42, 43, 44, 45, 46, 47, 48, 49, 50, 51, 52, 53, 54, 55, 56, 57, 58, 59, 50, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 tubes.

In some embodiments, each tube of the plurality of tubes may have the same length, or different tubes of the plurality of tubes may have different lengths. The average length of a tube may be about 5 nm to about 5 μm, such as about 5 nm to about 4 μm, about 5 nm to about 3 μm, about 5 nm to about 2 μm, or about 5 nm to about 1 μm. The average length of a tube may be about 50 nm to about 5 μm, such as about 50 nm to about 4 μm, about 50 nm to about 3 μm, about 50 nm to about 2 μm, or about 50 nm to about 1 μm. The average length of a tube may be about 100 nm to about 5 μm, such as about 100 nm to about 4 μm, about 100 nm to about 3 μm, about 100 nm to about 2 μm, or about 100 nm to about 1 μm. The average length of a tube may be about 300 nm to about 5 μm, such as about 300 nm to about 4 μm, about 300 nm to about 3 μm, about 300 nm to about 2 μm, or about 300 nm to about 1 μm. The average length of a tube may be about 500 nm to about 5 μm, such as about 500 nm to about 4 μm, about 500 nm to about 3 μm, about 500 nm to about 2 μm, or about 500 nm to about 1 μm.

In some embodiments, each tube of the plurality of tubes may have the same diameter, width, and/or height, or different tubes of the plurality of tubes may have different diameters, widths, and/or heights. The average diameter, width, and/or height of a tube may be about 5 nm to about 5 μm, such as about 5 nm to about 4 μm, about 5 nm to about 3 μm, about 5 nm to about 2 μm, about 5 nm to about 1 μm, about 5 nm to about 900 nm, about 5 nm to about 800 nm, about 5 nm to about 700 nm, about 5 nm to about 600 nm, about 5 nm to about 500 nm, about 5 nm to about 400 nm, about 5 nm to about 300 nm, about 5 nm to about 200 nm, or about 5 nm to about 100 nm. The average diameter, width, and/or height of a tube may be about 50 nm to about 5 μm, such as about 50 nm to about 4 μm, about 50 nm to about 3 μm, about 50 nm to about 2 μm, about 50 nm to about 1 μm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 200 nm, or about 50 nm to about 100 nm.

In some embodiments, each tube of the plurality of tubes may have the same internal diameter, internal width, and/or internal height, or different tubes of the plurality of tubes may have different internal diameters, widths, and/or heights. The average internal diameter, internal width, and/or internal height of a tube may be about 5 nm to about 4 μm, such as about 5 nm to about 3 μm, about 5 nm to about 2 μm, about 5 nm to about 1 μm, about 5 nm to about 900 nm, about 5 nm to about 800 nm, about 5 nm to about 700 nm, about 5 nm to about 600 nm, about 5 nm to about 500 nm, about 5 nm to about 400 nm, about 5 nm to about 300 nm, about 5 nm to about 200 nm, or about 5 nm to about 100 nm. The average internal diameter, internal width, and/or internal height of a tube may be about 20 nm to about 4 μm, such as about 20 nm to about 3 μm, about 20 nm to about 2 μm, about 20 nm to about 1 μm, about 20 nm to about 900 nm, about 20 nm to about 800 nm, about 20 nm to about 700 nm, about 20 nm to about 600 nm, about 20 nm to about 500 nm, about 20 nm to about 400 nm, about 20 nm to about 300 nm, about 20 nm to about 200 nm, or about 20 nm to about 100 nm. The average internal diameter, internal width, and/or internal height of a tube may be about 40 nm to about 4 μm, such as about 40 nm to about 3 μm, about 40 nm to about 2 μm, about 40 nm to about 1 μm, about 40 nm to about 900 nm, about 40 nm to about 800 nm, about 40 nm to about 700 nm, about 40 nm to about 600 nm, about 40 nm to about 500 nm, about 40 nm to about 400 nm, about 40 nm to about 300 nm, about 40 nm to about 200 nm, or about 40 nm to about 100 nm.

In some embodiments, a tube may comprise, for example, a polymer. The polymer may be a naturally-occurring polymer or a synthetic polymer. The polymer may be, for example, a nucleic acid (e.g., DNA) or protein.

Particles Comprising a DNA Scaffold

In some embodiments, the particle comprises a DNA scaffold, e.g., the particle may comprise a DNA origami scaffold (see, e.g., U.S. Pat. Nos. 8,554,489 and 7,842,793; U. S. Patent Application Publication Nos. 2013/0224859 and 2010/0216978; and PCT Patent Application Publication No. 2014/170898, each of which is hereby incorporated by reference).

In some embodiments, the particle may comprise a DNA scaffold, and the DNA scaffold may comprise at least one tube or a plurality of tubes as described herein. For example, the DNA scaffold may comprise at least one substantially hexagonal tube (see, e.g., U. S. Patent Application Publication No. 2013/0224859, hereby incorporated by reference).

In some embodiments, the DNA scaffold may comprise a honeycomb or lattice, such as a hexagonal lattice or a square lattice (see, e.g., U.S. Pat. No. 8,554,489, hereby incorporated by reference).

In some embodiments, the particle comprises a DNA scaffold, and the DNA scaffold does not comprise a tube. For example, the DNA scaffold may comprise a three-dimensional shape, such as a polyhedron, and the agent may be bound to the interior surface of the shape.

In some embodiments, the DNA scaffold may comprise a polyhedron, such as a regular polyhedron. The DNA scaffold may comprise a tetrahedron, such as a regular tetrahedron. The DNA scaffold may comprise a hexahedron, such as a cuboid, rectangular cuboid, or cube. The DNA scaffold may comprise an octahedron, such as a regular octahedron. The DNA scaffold may comprise a dodecahedron, such as a regular dodecahedron. The DNA scaffold may comprise an icosahedron, such as a regular icosahedron.

In some embodiments, the DNA scaffold may comprise a pyramid, such as a triangular pyramid, rhombic pyramid, rectangular pyramid, square pyramid, pentagonal pyramid, hexagonal pyramid, heptagonal pyramid, or octagonal pyramid. The DNA scaffold may comprise a right pyramid or an oblique pyramid.

In some embodiments, the DNA scaffold may comprise a prism, such as a triangular prism, rectangular prism, square prism, pentagonal prism, hexagonal prism, heptagonal prism, or octagonal prism. The DNA scaffold may comprise a right prism, an oblique prism, or a truncated prism.

In some embodiments, the DNA scaffold may comprise a length, width, and height, wherein no single dimension is more than 5 times larger than any other dimension. For example, no single dimension may be more than 4 times larger than any other dimension or no single dimension may be more than 3 times larger than any other dimension. Such configurations are favorable, for example, for intravenous administration of the particle because oblong particles may not flow as well in a patient's bloodstream.

In some embodiments, in some embodiments, the agent is bound to the DNA scaffold. In some embodiments, the agent is bound to a nucleic acid comprising a nucleotide sequence that is complementary to a nucleotide sequence on the DNA scaffold, i.e., the nucleotide sequence has at least about 95%, 96%, 97%, 98%, 99%, or 100% sequence identity with the reverse complement of the nucleotide sequence of the DNA scaffold. Thus, the agent may be bound to a surface of the particle by hybridizing the nucleic acid to the DNA scaffold.

Particles Comprising a Shield

In some embodiments, a particle may comprise a core subparticle and a shield, e.g., wherein the shield inhibits binding elements bound to the core subparticle from interacting with proteins or molecules on the surface of a cell. The shield may comprise a plurality of shield components. The core subparticle may comprise silica. For example, the core subparticle may comprise a silica surface. The core subparticle may comprise gold, silicon, or a polymer. For example, the core subparticle may comprise a gold, silicon, or polymer surface.

In some embodiments, a particle comprising an inner core subparticle and having a shield comprising a plurality of shield components attached to the core subparticle may comprise a core subparticle comprising a silica surface, such as a solid silica subparticle, a porous silica subparticle, or a silica nanoshell having a non-silica interior. The core subparticle may comprise a non-silica core material, such as silicon or gold, coated with silica. The shield components may be in the form of shield subparticles that are smaller than the core subparticle, such as nanospheres, and may comprise silica or a different material, such as gold or a polymer. The material of the surface of the core subparticle and of the shield components may be selected to be different to allow different coupling chemistry to be used to couple further components or species to the surfaces. The core subparticle may comprise a surface moiety having a reactive group, and the shield components may comprise a functional group capable of reaction with the reactive group to form a covalent bond between the surface of the core subparticles and the surface of the shield components or subparticles, as described herein.

In some embodiments, a binding element may be provided on the surface of the core subparticle but to a lesser extent, or preferably not at all, on the surface of the shield components. For example, an binding element may be attached to the surface of a silica core subparticle by a bond (e.g., an ionic, covalent, or electrostatic interaction) that forms preferentially (or exclusively) with the silica core subparticle and not with the shield subparticles, e.g., having a gold surface instead of a silica surface.

In some embodiments, such a particle may comprise a silica core, such as a substantially spherical silica core, and a shield comprising a plurality of gold nanoparticles on the surface of the silica core, the gold nanoparticles having a cross-sectional dimension smaller than a cross-sectional dimension of the core, such as the diameter of the core. The gold nanoparticles may be substantially spherical. The core subparticle may be solid and non-porous or may have a porous surface. Formation of the silica core and of gold nanoparticles on the core may be achieved, for example, as described in in U.S. Pat. No. 6,344,272, Sadtler and Wei, Chem. Comm. 1604-5 (2002); Meuhlig et al., ACS Nano, 5(8):6586-6592 (201 1) (each of which is hereby incorporated by reference herein in its entirety). For example, gold nanoparticles may be adsorbed onto an amine-coated silica core by means of electrostatic attraction, or may be linked to a silica core having thiol groups conjugated to the silica surface that then bond to the gold surface of the gold nanoparticles.

In some embodiments, a linker group may be provided between the silica of a core subparticle comprising silica and thiol groups for attaching a shield component to the core subparticle. The linker may have a length selected to set a maximum distance between a silica surface and a thiol group (or, when the thiol is attached to a gold surface, between the silica surface and the gold surface). In this way, the distance between the surface of the silica subparticle and the gold subparticle can be varied over a range of distances, potentially allowing a greater number of linkages (e.g., because more gold subparticles can be packed at a greater distance from the core silica subparticle), and/or strengthen the association between the silica and gold subparticles (e.g., because at shorter distances, more linkages from the surface of the silica subparticle may be able to interact with the same gold subparticle, reinforcing the association). A linker may comprise an alkylene chain whose length can be selected to vary the distance between the surface of the core subparticle and a shield subparticle.

In some embodiments, the core subparticle may have a cross-sectional dimension, such as the diameter of a spherical or cylindrical subparticle, of 50 nm to 4 μm, such as 50 nm to 200 nm, 100 nm to 500 nm, 200 nm to 1 μm, or 500 nm to 4 μm.

In some embodiments, particles may be assembled from a range of core subparticle diameters and shield subparticle diameters. The available surface area of the core subparticle for scavenging of a protein may depend on the diameter of the shield subparticles and the effective height above the surface of the core subparticle needed for binding of the binding element/protein complex to the surface, including the effective extent above the surface of any linker between the surface and the binding element.

In some embodiments, the number of binding elements that may be bound to a core subparticle may be calculated based on the surface area of the subparticle. Analogously, the number of target proteins that may be bound to the core subparticle may be calculated in a similar fashion. Such calculations may be confirmed, for example, by in vitro studies of protein binding, and may be used to predict the dose of particles that may be needed to scavenge a selected number of target proteins (or, in some embodiments, the effective dose of particles or of a formulation containing them for removing a number or reducing a concentration of target proteins from a system such as an in vitro system or from the circulation of a patient in treatment of disease).

In some embodiments, a particle may comprise an available surface area for the capture of a target protein of 0.01 μm 2 to 50 μm 2, such as 0.01 μm 2 to 0.1 μm2, 0.05 μm2 to 0.5 μm2, 0.1 μm2 to 1.0 μm2, 0.5 μm 2 to 5 μm2, 1.0 μm2 to 10 μm 2, 5 μm2 to 25 μm 2, or 10 μm 2 to 50 μm 2. For a selected loading of binding element per unit area of a core subparticle surface, a maximum dose of particles may be established as suitable to scavenge a desired quantity of target proteins based on the core and shield subparticle diameters.

In some embodiments, a cross-sectional dimension, such as the diameter, of the shield subparticle may be a multiple of a cross-sectional dimension, such as the diameter, of the core particle. The multiple may be, for example, 0.01 to 0.5, such as 0.02 to 0.2, such as 0.05 to 0.1.

Particles Comprising Subparticles

In some embodiments, a particle may comprise a core subparticle and a plurality of protecting subparticles. The particle may comprise a shield and the shield may comprise the plurality of protecting subparticles. The binding element may be bound to a surface of a core subparticle, e.g., wherein the surface of a core subparticle is an inner surface. The plurality of protecting subparticles may be configured to inhibit an interaction of a protein with a second member of a specific binding pair, e.g., when the protein is bound to the particle. The plurality of protecting subparticles may be configured to inhibit an interaction between a protein and a cell, such as a mammalian cell, e.g., when the protein is bound to the binding element on particle.

In some embodiments, the protecting subparticles may define an outer surface. In some embodiments, the binding element is not bound to the surface of the protecting subparticles.

In some embodiments, a core subparticle is preferably large enough to bind to more than one molecule of an binding element. For example, a core subparticle may be about 20 nm to about 4 μm in size, such as about 50 nm to about 2 μm in size. A core subparticle may be about 100 nm to about 1000 nm, about 100 nm to about 800 nm, about 100 nm to about 600 nm, about 100 nm to about 400 nm, about 100 nm to about 200 nm, about 200 nm to about 1000 nm, about 200 nm to about 800 nm, about 200 nm to about 600 nm, about 200 nm to about 400 nm, about 400 nm to about 1000 nm, about 400 nm to about 800 nm, about 400 nm to about 600 nm, about 600 nm to about 1000 nm, or about 600 nm to about 800 nm in size. A core subparticle may be about 100 nm to about 4 μm, 100 nm to about 3 μm, 100 nm to about 2 μm, about 200 nm to about 4 μm, 200 nm to about 3 μm, 200 nm to about 2 μm, about 400 nm to about 4 μm, 400 nm to about 3 μm, 400 nm to about 2 μm, about 600 nm to about 4 μm, 600 nm to about 3 μm, 600 nm to about 2 μm, about 800 nm to about 4 μm, 800 nm to about 3 μm, or 800 nm to about 2 μm in size.

In some embodiments, a core subparticle may comprise metal, gold, alumina, glass, silica, silicon, starch, agarose, latex, plastic, polyacrylamide, methacrylate, a polymer, or a nucleic acid. In some embodiments, a core subparticle comprises silicon, such as porous silicon.

In some embodiments, a core subparticle may be any shape (e.g., cubic, pyramidal, conic, spherical, cylindrical, disk, tetrahedral, hexahedral, octahedral, dodecahedral, or icosahedral) or a core subparticle may lack a defined shape. A particle may comprise 1 core subparticle. For example, the core subparticle may be a particle of U.S. Pat. No. 7,368,295 or 8,920,625 (each of which is hereby incorporated by reference in its entirety), which is further bound to a plurality of protecting subparticles.

In some embodiments, a particle may comprise a plurality of core subparticles, such as 2 to 300 core subparticles, 2 to 200 core subparticles, 2 to 150 core subparticles, 2 to 100 core subparticles, 2 to 80 core subparticles, or 2 to 42 core subparticles. In embodiments in which a particle comprises a plurality of core subparticles, each of the core subparticles are preferentially substantially spherical. A particle comprising a plurality of spherical core subparticles allows for voids, thereby allowing the diffusion of soluble proteins through the interior of the particle. Nevertheless, core subparticles of various other shapes may allow for voids. A particle comprising a plurality of core subparticles may comprise core subparticles of varying shapes and sizes.

In some embodiments, a particle may comprise 1 to about 106 core subparticles, 1 to about 105 core subparticles, 1 to about 104 core subparticles, 1 to about 1000 core subparticles, 1 to about 100 core subparticles, or 1 to about 10 core subparticles. A particle may comprise 2 to about 106 core subparticles, 2 to about 105 core subparticles, 2 to about 104 core subparticles, 2 to about 1000 core subparticles, 2 to about 100 core subparticles, or 2 to about 10 core subparticles. A particle may comprise about 10 to about 106 core subparticles, about 10 to about 105 core subparticles, about 10 to about 104 core subparticles, about 10 to about 1000 core subparticles, or about 10 to about 100 core subparticles.

In some embodiments, the core subparticles of a plurality of core subparticles may be connected by linkers (e.g., covalent linkers). For example, each core subparticle of a plurality of core subparticles may be connected to another core subparticle by a linker. In some embodiments, a core subparticle may comprise pores, i.e., a core subparticle may be porous.

In some embodiments, a protecting subparticle may comprise metal, gold, alumina, glass, silica, silicon, starch, agarose, latex, plastic, polyacrylamide, methacrylate, a polymer, or a nucleic acid. Some protecting subparticles are preferentially tethered to core subparticles by a linker, such as a covalent linker. Nevertheless, the protecting subparticles may be associated with one or more core subparticles without any covalent attachment. The protecting subparticles may be tethered to other protecting subparticles by linkers, such as by covalent linkers. For example, the protecting subparticles may form a web or net around the core subparticles, thereby sequestering the core subparticles within the particle.

In some embodiments, each protecting subparticle of the plurality of protecting subparticles are tethered to a core subparticle by a linker, such as a covalent linker. In some embodiments, some protecting subparticles of the plurality of protecting subparticles are tethered to a core subparticle, and each protecting subparticle of the plurality that is not directly tethered to a core subparticle is tethered to a protecting subparticle, i.e., such that each protecting subparticle of the plurality is either directly or indirectly tethered to a core subparticle. Thus, a particle may comprise a single layer of protecting subparticles {e.g., wherein substantially all of the protecting subparticles are directly tethered to one or more core subparticle(s)) or a particle may comprise more than one layer of protecting subparticles {e.g., wherein a substantial portion of the protecting subparticles are indirectly tethered to one or more core subparticle(s) through direct linkages with other protecting subparticles).

In some embodiments, a particle comprises a first layer of protecting subparticles comprising a first material and a second layer of protecting subparticles comprising a second material. For example, the first material may comprise silica or silicon and the second material may comprise gold. A particle may be assembled, for example, by linking the subparticles of the first layer of subparticles to one or more core subparticles and then linking the subparticles of the second layer of subparticles to the first layer of subparticles. The subparticles of the second layer may comprise a similar surface as the core subparticle(s), e.g., thereby allowing the subparticles of the first layer to link to both the core subparticle(s) and the subparticles of the second layer using similar chemistries.

In some embodiments, a particle may be assembled using a layer-by-layer method. For example, a particle may be formed by first linking a plurality of core subparticles. The plurality of core subparticles may be substantially homogenous, e.g., such that a linking molecule may cross-link the core subparticles. The plurality of subparticles may comprise at least two types of subparticles, e.g., with different shapes, sizes, and/or surfaces that allow for a desired feature, such as voids, within the particle. After linking the plurality of core subparticles, a plurality of protecting subparticles may be linked to the plurality of core subparticles. After linking the plurality of protecting subparticles to the core subparticles, a second plurality of protecting subparticles may be linked to the plurality of protecting subparticles. Nevertheless, a particle may be assembled in many different ways, and many different layer-by-layer strategies may be employed depending on the desired properties of the particle and the desired chemistries utilized to link the subparticles.

In some embodiments, a protecting subparticle may be about 10 nm to about 4 μm in size, such as about 10 nm to about 1 μm in size, or about 20 nm to about 500 nm in size. A protecting subparticle may be about 10 nm to about 200 nm, 10 nm to about 100 nm, about 10 nm to about 80 nm, about 10 nm to about 60 nm, about 10 nm to about 40 nm, about 10 nm to about 20 nm, 20 nm to about 200 nm, about 20 nm to about 100 nm, about 20 nm to about 80 nm, about 20 nm to about 60 nm, about 20 nm to about 40 nm, 30 nm to about 200 nm, about 40 nm to about 100 nm, about 40 nm to about 80 nm, about 40 nm to about 60 nm, 60 nm to about 200 nm, about 60 nm to about 100 nm, or about 60 nm to about 80 nm in size. A protecting subparticle may be about 100 nm to about 1000 nm, about 100 nm to about 800 nm, about 100 nm to about 600 nm, about 100 nm to about 400 nm, about 100 nm to about 200 nm, about 200 nm to about 1000 nm, about 200 nm to about 800 nm, about 200 nm to about 600 nm, about 200 nm to about 400 nm, about 400 nm to about 1000 nm, about 400 nm to about 800 nm, about 400 nm to about 600 nm, about 600 nm to about 1000 nm, or about 600 nm to about 800 nm in size. A protecting subparticle may be about 100 nm to about 4 μm, about 100 nm to about 3 μm, about 100 nm to about 2 μm, about 200 nm to about 4 μm, about 200 nm to about 3 μm, about 200 nm to about 2 μm, about 400 nm to about 4 μm, about 400 nm to about 3 μm, about 400 nm to about 2 μm, about 600 nm to about 4 μm, about 600 nm to about 3 μm, about 600 nm to about 2 μm, about 800 nm to about 4 μm, about 800 nm to about 3 μm, or about 800 nm to about 2 μm in size.

In some embodiments, a particle may comprise 1 to about 106 protecting subparticles, about 4 to about 106 protecting subparticles, about 10 to about 106 protecting subparticles, 1 to about 105 protecting subparticles, about 4 to about 105 protecting subparticles, about 10 to about 105 protecting subparticles, 1 to about 104 protecting subparticles, about 4 to about 104 protecting subparticles, about 10 to about 104 protecting subparticles, 1 to about 1000 protecting subparticles, about 4 to about 1000 protecting subparticles, about 10 to about 1000 protecting subparticles, 1 to about 100 protecting subparticles, about 4 to about 100 protecting subparticles, or about 10 to about 100 protecting subparticles.

In some embodiments, a core subparticle and a protecting subparticle may or may not have similar or identical shapes, sizes, and compositions. Nevertheless, a core subparticle varies from a protecting subparticle because (1) a binding element may be bound to a core subparticle whereas a binding element is preferentially not bound to a protecting subparticle, and (2) core subparticles are preferentially located in the interior of a particle whereas protecting subparticles may exist on the outer surface of a particle.

Substantially 2-Dimensional Particles

In some embodiments, a particle may be a 2-dimensional shape. For example, a particle may be a circle, ring, cross, fishbone, ellipse, triangle, square, pentagon, hexagon, heptagon, octagon, or star. A particle may be a star and the star may be a concave hexagon, concave octagon, concave decagon, or concave dodecagon. The shape may be a regular shape or an irregular shape. In some embodiments, a particle comprises a first side, a second side, and an edge.

In some embodiments, the first side and second side may be substantially the same shape. The first side and second side may comprise a length and a width. The edge may define a height, which is the distance between the first side and the second side. The width and length may be at least 4 times larger than the height, such as 4 to 1000 times larger, 6 to 100 times larger, 8 to 75 times larger, or 10 to 50 times larger than the height. The width and/or length may be 0.2 times to about 20 times larger than the height.

In some embodiments, an edge may comprise one or more concave or re-entrant portions. The binding element may be bound to the concave or re-entrant portions of the edge. A re-entrant portion is one in which the perimeter of the particle comprises two adjacent perimeter portions at an exterior angle between them of greater than 270 degrees, such as either side of the points of a star. In this way, the binding element may be shielded from contact with the membrane of a cell in contact with the particle.

In some embodiments, the first side and/or second side are substantially planar. In some embodiments, the first side and/or second side comprise a concave or re-entrant portion.

In some embodiments, the particle is in the form of a substantially flat star, e.g., with re-entrant portions between the points. A star may have 3, 4, 5, 6, 7, 8, 9, 10, 11, 12, 13, 14, 15, 16, or more points. The particle may comprise regular sides or irregular sides. In some embodiments, the particle is in the form of a cross or fishbone shape, e.g., comprising a backbone with arms extending on each side outwards from the backbone to define re-entrant surface portions between the arms. The arms of a cross or fishbone may further comprise lateral projections.

In some embodiments, the re-entrant edges between the points of the star or the arms of the cross or fishbone preferably extend a distance from the line joining the points such that a cell membrane cannot deform between the points so as to come into contact with the edges. For example, the number of points and the angle between them may determine the depth of the re-entrant edge portions between the points.

In some embodiments, particles suitable for use in the invention may be formed by nanofabrication, for example by nanoprinting or nanomoulding. For example, particles may be produced by the PRINT (“Particle Replication In Non-wetting Templates”) process (see, e.g., International patent application WO2007/024323; Perry, J. L. et al., Acc Chem Res. 44(10):990-998 (2011), each of which is hereby incorporated by reference). Particles may be produced by photolithography using known methods.

In some embodiments, an binding element may be bound to the edge of a particle and not bound to the first and second sides of a particle.

In some embodiments a desirable surface area per particle is in the range 0.2 to 25 μm2. The areas of the shielded edge portions of particles able to be fabricated by nanomoulding are therefore in a desirable range.

Binding Element

In some embodiments, the binding element bound to the surface of a particle is a small molecule, a macrocycle compound, a polypeptide, a peptidomimetic compound, an aptamer, a nucleic acid, or a nucleic acid analog. “Small molecule” as used herein, is meant to refer to an binding element, which has a molecular weight of less than about 6 kDa and most preferably less than about 2.5 kDa. Peptidomimetics can be compounds in which at least a portion of a subject polypeptide is modified, and the three dimensional structure of the peptidomimetic remains substantially the same as that of the subject polypeptide. Peptidomimetics may be analogues of a subject polypeptide of the disclosure that are, themselves, polypeptides containing one or more substitutions or other modifications within the subject polypeptide sequence. Alternatively, at least a portion of the subject polypeptide sequence may be replaced with a non-peptide structure, such that the three-dimensional structure of the subject polypeptide is substantially retained. In other words, one, two or three amino acid residues within the subject polypeptide sequence may be replaced by a non-peptide structure. In addition, other peptide portions of the subject polypeptide may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of humans or animals. It should be noted that peptidomimetics may or may not have similar two-dimensional chemical structures, but share common three-dimensional structural features and geometry. Each peptidomimetic may further have one or more unique additional binding elements.

In some embodiments, aptamers are short oligonucleotide sequences that can be used to recognize and specifically bind almost any molecule, including ceil surface proteins. The systematic evolution of ligands by exponential enrichment (SELEX) process is powerful and can be used to readily identify such aptamers. Aptamers can be made for a wide range of proteins of importance for therapy and diagnostics, such as growth factors and cell surface antigens.

In some embodiments, the binding element may be an antibody, or an antigen-binding portion thereof (i.e., an antibody fragment), wherein the antibody, or antigen-binding portion thereof, specifically binds to a target protein. The binding element may comprise an antibody, or an antigen-binding portion thereof, wherein the antibody, or antigen-binding portion thereof, specifically binds to a target protein. The term “antibody” includes a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody.

Production of Antibodies

As noted above, in some embodiments the binding element bound to the surface of the particle or particles is an antibody or antigen-binding fragment thereof. In some embodiments, a mammal, such as a mouse, a hamster or rabbit, may be immunized with an immunogenic form of a protein (e.g., a soluble T FR, a toxin, or a viral protein). Alternatively, immunization may occur by using a nucleic acid, which in vivo expresses a protein {e.g., a soluble protein) giving rise to the immunogenic response observed. Techniques for conferring immunogenicity on a protein or peptide include conjugation to carriers or other techniques well known in the art. For instance, a peptidyl portion of a polypeptide of the invention may be administered in the presence of adjuvant. The progress of immunization may be monitored by the detection of antibody titers in plasma or serum. ELISA or other immunoassays may be used with the immunogen as antigen to assess the concentrations of antibodies.

In some embodiments, following immunization, antisera reactive with a polypeptide of the invention may be obtained and, if desired, polyclonal antibodies isolated from the serum. To produce monoclonal antibodies, antibody producing cells (lymphocytes) may be harvested from an immunized animal and fused by standard somatic cell fusion procedures with immortalizing cells such as myeloma cells to yield hybridoma cells. In some embodiments, the hybridoma technique (originally developed by Kohler and Milstein, (1975) Nature, 256: 495-497), as the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with the polypeptides of the invention and the monoclonal antibodies isolated.

Positioning of a Binding Element on a Particle

In some embodiments, the geometry of the particle is such that the binding element has a reduced, or substantially reduced, ability to interact with a protein on the surface of a cell, such as an immune cell, blood cell, or lymphocyte. A binding element may have less than 50% (e.g., 45, 40, 35, 30, 25, 20, 15, 10, 9, 8, 7, 6, 5, 4, 3, 2, or 1%) of the ability to bind to a protein on a surface of a cell relative to a free, soluble form of the binding element.

In some embodiments, the soluble protein bound to the particle has a reduced, or substantially reduced, ability to interact with its cognate ligand (the second member of the specific binding pair). The protein may be bound to the particle by virtue of the binding element. A protein bound to a particle may have less than 50% (e.g., 45, 40, 35, 30, 25, 20, 15, 10, 9, 8, 7, 6, 5, 4, 3, 2, or 1%) of the ability to interact with its cognate ligand relative to the ability of an unbound protein. For example, a soluble virion bound to a particle described herein has less than 50 (e.g., 45, 40, 35, 30, 25, 20, 15, 10, 9, 8, 7, 6, 5, 4, 3, 2, or 1) % of the ability of free virion to interact with its cognate cell surface receptor(s) and infect a cell.

In some embodiments, the binding element may be bound to an inner surface of a particle (e.g., the pores of a porous particle or the inner surface of a tube). In some embodiment, the binding element can be bound to the outer surface of a particle, but is sterically precluded from interacting with a cell surface by way of one or more protrusions from the particle. In some embodiments, e.g., toroidal particles, the binding element is bound to the inner surface of the particle such that the binding element has a reduced, or substantially reduced, ability to interact with a protein on the surface of a cell and/or the soluble protein bound to the particle by virtue of the binding element has a reduced, or substantially reduced, ability to interact with its cognate ligand (the second member of the specific binding pair).

Clearance Agents and Coatings

In some embodiments, a particle comprises a clearance agent. The clearance agent may facilitate clearance of the particle through a biological pathway, such as by excretion in the urine, degradation, excretion by a hepatobiliary pathway, and/or phagocytosis. For example, the particle may comprise a reservoir, wherein the reservoir comprises a clearance agent. The reservoir may be a hole or void in the body of a particle, e.g., a void in the body of a porous silicon particle.

In some embodiments, for particles comprising pores, the reservoir may be a pore or the reservoir may be larger or smaller than the average pore size. A reservoir may consist of a recess in the body of a particle (e.g., a shallow recess), wherein the width or diameter of the recess is larger than the width or diameter of the average pore size. The width or diameter of a reservoir may be at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 90, 100, 110, 120, 130, 140, 150, 175, 200, 250, 300, 400, or even about 500 times as large as the width or diameter of the average pore size.

In some embodiments, the width or diameter of the reservoir may be about 2 times to about 10 times the width or diameter of the average pore size, such as about 2 times to about 8 times or about 2 times to about 6 times. The width or diameter of a reservoir may be about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 90, 100, 110, 120, 130, 140, 150, 175, 200, 250, 300, 400, or even about 500 times as large as the width or diameter of the average pore size.

In some embodiments, for particles comprising a DNA scaffold, a reservoir may be an interior region of the DNA scaffold. The reservoir (e.g., interior region) may be inaccessible to cells, e.g., the DNA scaffold may be constructed such that the scaffold sterically hinders cells from entering the interior region. In some embodiments, the reservoir (e.g., interior region) is inaccessible to extracellular proteins, e.g., the DNA scaffold may be constructed such that the scaffold sterically hinders extracellular proteins from entering the reservoir. The reservoir (e.g., interior region) may be inaccessible to antibodies. Nevertheless, the DNA scaffold may allow for the reservoir (e.g., interior region) to become accessible to cells and/or extracellular proteins after a predetermined period of time. For example, the DNA scaffold may comprise a biodegradable wall that may degrade after a predetermined period of time (e.g., by hydrolysis), thereby exposing the clearance agent to cells and/or extracellular proteins. The DNA scaffold may comprise a biodegradable latch that may degrade after a predetermined period of time (e.g., by hydrolysis), allowing the DNA scaffold to undergo a conformational change, thereby exposing the clearance agent to cells and/or extracellular proteins (see, e.g., PCT Patent Application Publication No. WO2014/170899, hereby incorporated by reference). Similarly, the DNA scaffold may comprise a reservoir that comprises and opening, as described below.

In some embodiments, the reservoir may comprise an opening. The opening may be covered by a cap or member, thereby inhibiting interactions between the clearance agent and cells and/or extracellular proteins (e.g., antibodies). The cap or member may comprise a polymer, such as a biodegradable polymer. The cap or member may degrade after a predetermined period of time (e.g., by hydrolysis), thereby exposing the clearance agent to cells and/or extracellular proteins. The cap or member may degrade (e.g., biodegrade) after exposure to a biological fluid (e.g., blood plasma or extracellular fluid) for about 1 day to about 5 years, such as about 1 day to about 4 years, about 1 day to about 3 years, or about 1 day to about 1 year.

In some embodiments, a predetermined period of time may be a period of time that the particle is in a liquid (e.g., an aqueous liquid). The predetermined period of time may be a period of in vivo residence of a particle (e.g., exposure to biological fluids, pH, enzymes, and/or temperatures). The predetermined period of time may be determined, at least in part, by the binding of the particle to a protein. For example, the particle may be configured such that the binding of a protein exposes the clearance agent to cells and/or extracellular proteins (see, e.g., PCT Patent Application Publication No. WO2014/170899, hereby incorporated by reference). The predetermined period of time may be about 1 day to about 5 years, such as about 1 day to about 3 years, or about 1 day to about 1 year.

In some embodiments, exemplary materials suitable for use as caps or membranes, are described in U.S. Pat. No. 7,918,842, which is hereby incorporated by reference. In general, these materials degrade or dissolve either by enzymatic hydrolysis or exposure to water in vivo or in vitro, or by surface or bulk erosion. Representative synthetic, biodegradable polymers include: poly(amides) such as poly(amino acids) and poly(peptides); poly(esters) such as poly(lactic acid), poly(glycolic acid), poly(lactic-co-glycolic acid), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), copolymers and mixtures thereof. Other polymers that may be used in caps or membranes include: poly(ethers) such as poly(ethylene oxide), poly(ethylene glycol), and poly(tetramethylene oxide); vinyl polymers-poly(acrylates) and poly(methacrylates) such as methyl, ethyl, other alkyl, hydroxyethyl methacrylate, acrylic and methacrylic acids, and others such as poly(vinyl alcohol), poly(vinyl pyrrolidone), and poly(vinyl acetate); poly(urethanes); cellulose and its derivatives such as alkyl, hydroxyalkyl, ethers, esters, nitrocellulose, and various cellulose acetates; poly(siloxanes); and any chemical derivatives thereof (substitutions, additions of chemical groups, for example, alkyl, alkylene, hydroxylations, oxidations, and other modifications routinely made by those skilled in the art), copolymers and mixtures thereof. In certain embodiments, the reservoir cap is formed from one or more cross-linked polymers, such as cross-linked polyvinyl alcohol.

In some embodiments, a particle comprises a coating. In some embodiments, the coating comprises a clearance agent. The coating may mask a clearance agent. The particle may comprise a first surface and a second surface; the binding element may be bound to the first surface; and the coating may cover at least a portion of the second surface. The first surface may be an interior surface or an inner surface, e.g., the first surface may be oriented such that the binding element has a reduced ability to bind to a molecule on a cell surface. Examples of an interior surface or inner surface include the inner walls of a pore, reservoir, or tube, the inner circumferential surface of a toroid, or the hollow of a concave surface. Other examples of an interior surface or inner surface include the outer surface of a particle, wherein the outer surface is protected from interactions with cells by one or more protrusions. The second surface may be an exterior surface or outer surface, e.g., the second surface may be oriented such that the coating can interact with a cell. In some embodiments, a particle may comprise one or more core subparticles and a plurality of protecting subparticles. The particle may comprise a shield and the shield may comprise the plurality of protecting subparticles. The first surface may be the surface of the one or more core particles and the second surface may be the surface of the protecting subparticles.

In some embodiments, a coating may inhibit interactions between particles, e.g., the coating may reduce the propensity of particles to form aggregates. The coating may inhibit interactions between a particle and cells, e.g., by presenting a biologically-inert surface. The coating may inhibit non-specific interactions with extracellular molecules, e.g., non-specific adsorption of proteins. A coating may inhibit specific interactions with cells or extracellular molecules, e.g., a coating may disfavor or delay the excretion or phagocytosis of a particle. A coating may target a particle for excretion or phagocytosis. A coating or other feature (e.g., an “excretion-inducing compound”) that targets a particle for excretion or phagocytosis may be masked by a coating (e.g., a second coating) that delays the excretion or phagocytosis of the particle, e.g., to promote maintenance of the particles in the bloodstream for a predetermined amount of time.

In some embodiments, a coating may comprise a plurality of elongated coating molecules bound at one end to the surface of the particle. A coating may inhibit interactions between a protein bound to a particle and a second member of the specific binding pair that includes the protein. A coating may inhibit interactions between a protein bound to a particle and a cell. A binding element may be oriented on a particle relative to a coating such that the binding element has a reduced ability to bind to a molecule on the surface of a cell. A binding element may be oriented on a particle relative to a coating such that the binding element has a reduced ability to bind to a target protein on the surface of a cell. A binding element may be oriented on a particle relative to a coating such that the coating sterically inhibits the binding element from binding to a molecule on the surface of a cell. A binding element may be oriented on a particle such that the coating sterically inhibits the binding element from binding to a target protein on the surface of a cell. A coating may be oriented on a particle such that the binding element of the particle has a reduced ability to bind to a molecule on the surface of a cell. A coating may reduce the ability of the binding element of a particle to activate a cell surface receptor protein, relative to the ability of a natural ligand of the cell surface receptor protein.

In some embodiments, a particle may comprise a second coating, e.g., wherein the second coating consists of a second plurality of coating molecules. A particle may comprise a second plurality of coating molecules. The second coating and/or second plurality of coating molecules may decrease the clearance of the particle in vivo, e.g., by masking the coating and/or plurality of coating molecules. The second coating and/or second plurality of coating molecules may be biodegradable, e.g., to expose the coating and/or plurality of coating molecules to cells and/or extracellular proteins after a predetermined period of time. The second coating and/or second plurality of coating molecules may comprise a biodegradable polymer, e.g., each molecule of the second plurality of coating molecules may comprise a biodegradable polymer.

In some embodiments, the particle comprises a first surface {e.g., an interior surface) and a second surface {e.g., an exterior surface or outer surface); the binding element is bound to the first surface; and the coating covers at least a portion of the second surface. The orientation of the first surface may reduce the ability of the binding element to interact with molecules on a cell surface. The orientation of the second surface may permit interactions between the coating and cells, extracellular molecules, and/or different particles. An “interaction” between the coating and cells, extracellular molecules, and/or different particles may be a weak, neutral, or unfavorable interaction, e.g., to disfavor stable binding of the particle to a cell, extracellular molecule, or other particle. Alternatively, an interaction between the coating and either cells and/or extracellular molecules may be a specific or designed interaction, e.g., to favor clearance of the particle through a biological pathway, such as phagocytosis. In certain preferred embodiments, the second surface is substantially free of binding element. In certain preferred embodiments, the first surface is substantially free of coating. In certain preferred embodiments, the coating covers substantially all of the second surface.

In some embodiments, the particle comprises a first surface, e.g., an interior surface, and a second surface, e.g., an exterior surface or outer surface; the binding element is bound to the first surface and the second surface; and the coating covers at least a portion of the second surface. In such embodiments, the coating (and/or a second coating) may inhibit interactions between the binding element and molecules on a cell surface. In certain preferred embodiments, the coating covers substantially all of the second surface.

In some embodiments, the particle comprises a first surface (e.g., an interior surface) and a second surface (e.g., an exterior surface or outer surface); the binding element is bound to the first surface; and the coating covers at least a portion of the first surface and at least a portion of the second surface. In such embodiments, the coating preferably does not affect the ability of the binding element to specifically bind to a protein. In certain preferred embodiments, the coating covers substantially all of the second surface.

In some embodiments, the particle comprises a surface; the binding element is bound to the surface; and the coating covers at least a portion of the surface. In such embodiments, the coating may not affect the ability of the binding element to specifically bind to a protein. The coating may allow for some of the binding element to specifically bind to a protein and inhibit interactions between some of the binding element and protein. The coating may inhibit interactions between the binding element and molecules on a cell surface. In certain preferred embodiments, the coating covers substantially all of the surface.

In some embodiments, the particle comprises a coating that covers at least a portion of the second surface and a second coating that covers at least a portion, such as substantially all, of the coating on the second surface. In such embodiments the coating may comprise a clearance agent, such as an “excretion-inducing compound” to target a particle for excretion or phagocytosis. Such a coating may comprise beta-cyclodextrin. The second coating may comprise a material, e.g., a second plurality of coating molecules, to inhibit interaction with cells and/or inhibit non-specific interactions with extracellular molecules, e.g., non-specific adsorption of proteins. The second coating may be biodegradable, e.g., to expose the coating on the second surface to cells and/or extracellular proteins after a predetermined period of time. For example, in a particle comprising one or more core subparticles and a plurality of protecting sub-particles, wherein a binding element is bound to the surface on the core subparticle(s) (i.e., the first surface), at least a portion of the surface of the protecting subparticles (i.e., the second surface) comprises a coating, for example a coating comprising either a clearance agent or a coating comprising a material to inhibit interaction with cells and/or to inhibit non-specific interaction with extracellular molecules.

In some embodiments, a coating may comprise coating molecules, e.g., a coating may consist of a plurality of coating molecules or a coating may consist of a population of coating molecules. As used herein, the terms “plurality of coating molecules” and “population of coating molecules” each refer to a coating. The term “coating,” however, may refer to additional compositions, such as a hydrogel. A coating molecule may be a clearance agent (and thus, a clearance agent may be a coating molecule).

In some embodiments, a particle may comprise a plurality of coating molecules. The particle may comprise a surface and a plurality of binding elements bound to the surface, and at least one molecule of the plurality of coating molecules may be bound to the surface. For example, all or substantially all of the molecules of the plurality of coating molecules may be bound to the surface.

The particle may comprise a surface and a second surface, wherein a plurality of binding elements bound to the surface, and at least one molecule of the plurality of coating molecules may be bound to the second surface. For example, all or substantially all of the molecules of the plurality of coating molecules may be bound to the second surface. In some embodiments, some of the molecules of the plurality of coating molecules are bound to the surface and some of the molecules of the plurality of coating molecules are bound to the second surface. In some embodiments, the coating molecules increase the clearance of the particle in vivo. For example, the coating molecules may comprise a pathogen-associated molecular pattern.

In some embodiments, the particles described herein have a coating comprising an excretion-inducing compound, which facilitates the removal of the particles from the circulation, e.g., via the kidneys, liver/intestines {e.g., via bile), or phagocytosis {e.g., by antigen-presenting cells). A plurality of coating molecules may be a plurality of excretion-inducing compounds. For example, in embodiments in which the particles are toroidal, the inner circumferential surface {e.g., a first surface) may comprise a binding element and the outer surface {e.g., a second surface) may comprise a compound that induces the clearance of the particles, e.g., by the kidneys, liver, or macrophages. In some embodiments, the excretion-inducing compound is programmed. That is, the compound can be covered with a coating that degrades {e.g., through the action of enzymes, hydrolysis, or gradual dissolution) over time {e.g., a predetermined amount of time) eventually exposing the excretion-inducing compound or other feature that increases the rate of clearance. The coating may degrade after exposure to a biological fluid {e.g., blood plasma or extracellular fluid) for about 1 day to about 5 years, such as about 1 day to about 3 years, or about 1 day to about 1 year. Thus, the in vivo residence of a particle may be modified and/or controlled.

In some embodiments, a coating may comprise an organic polymer, such as polyethylene glycol (PEG). An organic polymer may be attached to a particle, e.g., attached to a surface of the particle. The organic polymer may include PEG, polylactate, polylactic acids, sugars, lipids, polyglutamic acid, polyglycolic acid (PGA), polylactic acid (PLA), poly(lactic-co-glycolic acid) (PLGA), polyvinyl acetate (PVA), and combinations thereof. In certain embodiments, the particle is covalently conjugated with PEG, which discourages adsorption of serum proteins, facilitates efficient urinary excretion and decreases aggregation of the particle (see, e.g., Burns et al., Nano Letters, 9(1):442-448 (2009) and U.S. Patent Application Publication Nos. 2013/0039848 and 2014/0248210, each of which is hereby incorporated by reference).

In one embodiment, the coating comprises at least one hydrophilic moiety, for example, Pluronic® type polymers (a nonionic polyoxyethylene-polyoxypropylene block co-polymer with the general formula HO(C2H40)a(-C3H60)b(C2H40)aH), a triblock copolymer poly(ethylene glycol-b-(DL-lactic acid-co-glycolic acid)-b-ethylene glycol) (PEG-PLGA-PEG), a diblock copolymer polycaprolactone-PEG (PCL-PEG), poly(vinylidene fluoride)-PEG (PVDF-PEG), poly(lactic acid-co-PEG) (PLA-PEG), poly(methyl methacrylate)-PEG (PMMA-PEG) and so forth. In an embodiment with such a moiety, the hydrophilic moiety is a PEG moiety such as: a

[Methoxy(Polyethyleneoxy)Propyl]-Trimethoxysilane (e.g., CH3(OC2H4)6-9(CH2)OSi(OCH3)3), a [Methoxy(Polyethyleneoxy)Propyl]-Dimethoxysilane (e.g., CH3(OC2H4)6-9(CH2)OSi(OCH3)2), or a [Methoxy(Polyethyleneoxy)Propyl]-Monomethoxysilane (e.g., CH3(OC2H4)6-9(CH2)OSi(OCH3)). Suitable coatings are described, for example, in U.S. Patent Application Publication No. 2011/0028662 (hereby incorporated by reference).

In some embodiments, the coating may include a polyhydroxylated polymer, such as natural polymers or hydroxyl-containing polymers including multiply-hydroxylated polymers, polysaccharides, carbohydrates, polyols, polyvinyl alcohol, poly amino acids such as polyserine, or other polymers such as 2-(hydroxyethyl)methacrylate, or combinations thereof. In some embodiments, the polyhydroxylated polymers are polysaccharides. Polysaccharides include, mannan, pullulan, maltodextrin, starches, cellulose, and cellulose derivatives, gums, xanthan gum, locust bean gum, or pectin, combinations thereof (see, e.g., U.S. Patent Application Publication No. 2013/0337070, hereby incorporated by reference).

In some embodiments, the coating comprises a zwitterionic polymer (see, e.g., U.S. Patent Application Publication Nos. 2014/0235803, 2014/0147387, 2013/0196450, and 2012/0141797; and U.S. Pat. No. 8,574,549, each of which is hereby incorporated by reference).

In some embodiments, other suitable coatings include poly-alpha hydroxy acids (including polyactic acid or polylactide, polyglycolic acid, or polyglycolide), poly-beta hydroxy acids (such as polyhydroxybutyrate or polyhydroxyvalerate), epoxy polymers (including polyethylene oxide (PEO)), polyvinyl alcohols, polyesters, polyorthoesters, polyamidoesters, polyesteramides, polyphosphoesters, and polyphosphoester-urethanes. Examples of degradable polyesters include: poly(hydroxyalkanoates), including poly(lactic acid) or (polylactide, PLA), poly(glycolic acid) or polyglycolide (PGA), poly(3-hydroxybutyrate), poly(4-hydroxybutyrate), poly(3-hydroxy valerate), and poly(caprolactone), or poly(valerolactone). Examples of polyoxaesters include poly(alkylene oxalates) such as poly(ethylene oxalate)) and polyoxaesters containing amido groups. Other suitable coating materials include poly ethers including polyglycols, ether-ester copolymers (copoly (ether-esters)) and polycarbonates. Examples of biodegradable polycarbonates include polyorthocarbonates, polyiminocarbonates, polyalkylcarbonates such as poly(trimethylene carbonate), poly(1,3-dioxan-2-one), poly(p-dioxanone), poly(6,6-dimethyl-1,4-dioxan-2-one), poly(1,4-dioxepan-2-one), and poly(1,5-dioxepan-2-one). Suitable biodegradable coatings can also include polyanhydrides, polyimines (such as poly(ethylene imine) (PEI)), polyamides (including poly-N-(2-hydroxypropyl)-methacrylamide), poly(amino acids) (including a polylysine such as poly-L-lysine, or a polyglutamic acid such as poly-L-glutamic acid), polyphosphazenes (such as poly(phenoxy-co-carboxylatophenoxy phosphazene), polyorganophosphazenes, polycyanoacrylates and polyalkylcyanoacrylates (including polybutylcyanoacrylate), polyisocyanates, and polyvinylpyrrolidones. The chain length of a polymeric coating molecule may be about 1 to about 100 monomer units, such as about 4 to about 25 units.

In some embodiments, a particle may be coated with a naturally occurring polymer, including fibrin, fibrinogen, elastin, casein, collagens, chitosan, extracellular matrix (ECM), carrageenan, chondroitin, pectin, alginate, alginic acid, albumin, dextrin, dextrans, gelatins, mannitol, n-halamine, polysaccharides, poly-1,4-glucans, starch, hydroxy ethyl starch (HES), dialdehyde starch, glycogen, amylase, hydroxyethyl amylase, amylopectin, glucoso-glycans, fatty acids (and esters thereof), hyaluronic acid, protamine, polyaspartic acid, polyglutamic acid, D-mannuronic acid, L-guluronic acid, zein and other prolamines, alginic acid, guar gum, and phosphorylcholine, as well as co-polymers and derivatives thereof. The coating may also comprise a modified polysaccharide, such as cellulose, chitin, dextran, starch, hydroxyethyl starch, polygluconate, hyaluronic acid, and elatin, as well as co-polymers and derivative thereof.

In some embodiments, a particle may be coated with a hydrogel. The hydrogel can be formed, for example, using a base polymer selected from any suitable polymer, such as poly(hydroxyalkyl (meth)acrylates), polyesters, poly(meth)acrylamides, poly(vinyl pyrrolidone), or polyvinyl alcohol. A cross-linking binding element can be one or more of peroxides, sulfur, sulfur dichloride, metal oxides, selenium, tellurium, diamines, diisocyanates, alkyl phenyl disulfides, tetraalkyl thiuram disulfides, 4,4′-dithiomorpholine, p-quinine dioxime and tetrachloro-p-benzoquinone. Also, boronic acid-containing polymers can be incorporated in hydrogels, with optional photopolymerizable groups.

In some embodiments, the coating comprises a material that is approved for use by the U.S. Food and Drug Administration (FDA). These FDA-approved materials include polyglycolic acid (PGA), polylactic acid (PLA), Polyglactin 910 (comprising a 9:1 ratio of glycolide per lactide unit, and known also as VICRYL™), polyglyconate (comprising a 9:1 ratio of glycolide per trimethylene carbonate unit, and known also as MAXON™), and polydioxanone (PDS).

In some embodiments, the attachment of a coating to a particle may be accomplished by a covalent bond or a non-covalent bond, such as by ionic bond, hydrogen bond, hydrophobic bond, coordination, adhesive, or physical absorption or interaction.

In some embodiments, nanoparticle coating methods include dry and wet approaches. Dry methods include: (a) physical vapor deposition (Zhang, Y. et al., Solid State Commun. 115:51 (2000)), (b) plasma treatment (Shi, D. et al., Appl. Phys. Lett. 78: 1243 (2001); Vollath, D. et al., J. Nanoparticle Res. 1:235 (1999)), (c) chemical vapor deposition (Takeo, O. et al., J. Mater. Chem. 8: 1323 (1998)), and (d) pyrolysis of polymeric or non-polymeric organic materials for in situ precipitation of nanoparticles within a matrix (Sglavo, V. M. et al., J. Mater Sci. 28:6437 (1993)). Wet methods for coating particles include: (a) sol-gel processes and (b) emulsification and solvent evaporation techniques (Cohen, H. et al., Gene Ther. 7: 1896 (2000); Hrkach, J. S. et al., Biomaterials 18:27 (1997); Wang, D. et al., J. Control. Rel. 57:9 (1999)). A coating may be applied by electroplating, spray coating, dip coating, sputtering, chemical vapor deposition, or physical vapor deposition. Additionally, methods for coating various nanoparticles with polysaccharides are known in the art (see, e.g., U.S. Pat. No. 8,685,538 and U.S. Patent Application Publication No. 2013/0323182, each of which is hereby incorporated by reference).

In some embodiments, the particles may be adapted to facilitate clearance by renal excretion. Renal clearance for subjects with normal renal function generally requires particles with at least one dimension that is less than 15 nm (see, e.g., Choi, H. S., et al., Nat Biotechnol 25(1): 1165 (2007); Longmire, M. et al., Nanomedicine 3(5):703 (2008)). Nevertheless, larger particles may be excreted in the urine. For embodiments in which a particle is too large for renal clearance, the particle may nevertheless be cleared following in vivo degradation to a smaller size.

In some embodiments, the particle is adapted to facilitate clearance by phagocytosis. For example, the particle may comprise a clearance agent, wherein the clearance agent comprises a pathogen-associated molecular pattern, e.g., for recognition by macrophages. Pathogen-associated molecular patterns (PAMPs) include unmethylated CpG DNA (bacterial), double-stranded RNA (viral), lipopolysacharride (bacterial), peptidoglycan (bacterial), lipoarabinomannan (bacterial), zymosan (yeast), mycoplasmal lipoproteins such as MALP-2 (bacterial), flagellin (bacterial), poly(inosinic-cytidylic) acid (bacterial), lipoteichoic acid (bacterial), and imidazoquinolines (synthetic). In some embodiments, the PAMP clearance agent is masked such that macrophages do not engulf the particle prior to the binding of the particle to one or more target proteins. For example, a PAMP clearance agent may be masked by any one of the aforementioned coatings (e.g., a polymeric coating, such as a biodegradable polymeric coating). Macrophages can engulf particles as large as 20 pir (see, e.g., Cannon, G. J. and Swanson, J. A., J. Cell Science 101:907-913 (1992); Champion, J. A., et al., Pharm Res 25(8): 1815-1821 (2008)). In some embodiments, a clearance agent that facilitates clearance by phagocytosis may cover a portion of an inner surface of a particle such that the clearance agent becomes exposed following degradation of the particle. The particle may comprise a plurality of clearance agents, e.g., PAMPs, that cover a portion of a surface. The surface may be exposed following degradation of the particle, allowing for clearance of the degraded particle. The clearance agent may cover a portion of a surface that overlaps a surface comprising an binding element. The clearance agent (e.g., PAMPs) may elicit an immune response against the particle, e.g., following the degradation of a second coating or following the degradation of the particle.

In some embodiments, an immune response directed against a clearance agent (e.g., PAMPs) may outcompete an immune response directed against the binding element and/or binding element/protein complex, thereby inhibiting or delaying the onset of an immune response directed against the binding element and/or binding element/protein complex. For example, degradation of a particle may expose both a clearance agent and an binding element (and/or binding element/protein complex) to leukocytes. A PAMP clearance agent may allow for rapid clearance of the degraded particle by macrophages, thereby delaying an immune response (e.g., B-cell mediated immune response) against the binding element and/or binding element/protein complex. In some embodiments, a clearance agent may be calreticulin, which induces phagocytosis.

In some embodiments, the coating molecule comprises a nucleic acid, e.g., for hybridizing with a coating molecule to a particle comprising a DNA scaffold. For example, a particle may comprise a nucleic acid and a coating molecule, wherein the coating molecule comprises a complementary nucleic acid that can hybridize with the nucleic acid, thereby forming a bond between the coating molecule and the particle (i.e., hydrogen bonds). The nucleic acid may comprise a nucleotide sequence and the complementary nucleic acid may comprise a complementary nucleotide sequence, e.g., wherein the nucleotide sequence has at least 95%, 96%, 97%, 98%, or 99% sequence i.e. identity with the reverse complement of the complementary nucleotide sequence. The nucleotide sequence may have 100% sequence i.e. identity with the reverse complement of the complementary nucleotide sequence.

In some embodiments, the melting temperature of the nucleic acid and complementary nucleic acid in physiological fluid (e.g., blood) is greater than body temperature (e.g., the body temperature of a subject, such as a human or mouse). For example, the melting temperature of the nucleic acid and complementary nucleic acid in physiological fluid is preferably greater than 37° C., such as greater than about 38° C., greater than about 39° C., greater than about 40° C., greater than about 41° C., greater than about 42° C., greater than about 43° C., greater than about 44° C., or greater than about 45° C. The melting temperature of the nucleic acid and complementary nucleic acid may be about 37° C. to about 120° C., such as about 38° C. to about 120° C., about 39° C. to about 120° C., about 40° C. to about 120° C., about 4FC to about 120° C., about 42° C. to about 120° C., about 43° C. to about 120° C., about 44° C. to about 120° C., about 45° C. to about 120° C., about 46° C. to about 120° C., about 47° C. to about 120° C., about 48° C. to about 120° C., about 49° C. to about 120° C., about 50° C. to about 120° C., about 38° C. to about 100° C., about 39° C. to about 100° C., about 40° C. to about 100° C., about 41° C. to about 100° C., about 42° C. to about 100° C., about 43° C. to about 100° C., about 44° C. to about 100° C., about 45° C. to about 100° C., about 46° C. to about 100° C., about 47° C. to about 100° C., about 48° C. to about 100° C., about 49° C. to about 100° C., or about 50° C. to about 100° C.

In some embodiments, the length of the nucleic acid of the reactive group, nucleotide sequence of the reactive group, complementary nucleic acid, and complementary nucleotide sequence is preferably greater than 9 nucleotides. The length of the nucleic acid of the reactive group, nucleotide sequence of the reactive group, complementary nucleic acid, and complementary nucleotide sequence may be greater than 10, 11, 12, 13, 14, 15, 16, 17, 18, 19, or 20 nucleotides. The length of the nucleic acid of the reactive group, nucleotide sequence of the reactive group, complementary nucleic acid, and complementary nucleotide sequence may be about 10 nucleotides to about 100 nucleotides, such as about 11 nucleotides to about 80 nucleotides, about 12 nucleotides to about 60 nucleotides, about 13 nucleotides to about 50 nucleotides, about 14 nucleotides to about 40 nucleotides, about 15 nucleotides to about 30 nucleotides, or about 16 nucleotides to about 25 nucleotides. The GC content of the nucleic acid, nucleotide sequence, complementary nucleic acid, and complementary nucleotide sequence may be about 10% to about 100%, such as about 40% to about 100%, about 45% to about 100%, about 50% to about 100%, about 55% to about 100%, about 40% to about 95%, about 45% to about 90%, about 50% to about 85%, or about 55% to about 80%.

In some embodiments, a particle may be cleared by an organism in about 1 day to about 5 years, such as about 1 day to about 3 years, or about 1 day to about 1 year.

Administration of Protein Binding Particles

In some embodiments, the disclosure contemplates administration of a pharmaceutical composition comprising a binding element that binds to, modulates, or degrades a protein, e.g., a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, to cells and tissues in vitro and/or in vivo. Administration in vivo includes administration to an animal model of disease, such as an animal model of cancer, or administration to a subject in need thereof. Suitable cells, tissues, or subjects include animals, such as companion animals, livestock, zoo animals, endangered species, rare animals, non-human primates, and humans. Exemplary companion animals include dogs and cats.

In some embodiments, for delivery in vitro, such as to and/or around cells or tissues in culture, compositions may be added to the culture media, such as to contact the microenvironment or contact soluble material in the culture media or to contact the cell or even to penetrate the cell. The desired site of activity influences the delivery mechanism and means for administering the compositions {e.g., particles described herein).

In some embodiments, for delivery in vivo, such as to cells or tissues in vivo (including to the microenvironment of cells and tissue) and/or to a subject in need thereof, numerous methods of administration are envisioned. The particular method may be selected based on the particle composition and the particular application and the patient. Various delivery systems can be used to administer binding elements of the disclosure. Any such methods may be used to administer any of the binding elements described herein. Methods of introduction can be enteral or parenteral, including but not limited to, intradermal, intramuscular, intraperitoneal, intramyocardial, intravenous, subcutaneous, pulmonary, intranasal, intraocular, epidural, and oral routes. A composition of the disclosure may be administered by any convenient route, for example, by infusion or bolus injection, by absorption through epithelial or mucocutaneous linings {e.g., oral mucosa, rectal and intestinal mucosa, etc.) and may be administered together (either concurrently or consecutively) with other biologically active binding elements. Administration can be systemic or local.

In certain embodiments, a composition is administered intravenously, such as by bolus inject or infusion. In certain embodiments, a composition is administered orally, subcutaneously, intramuscularly or intraperitoneally. In certain embodiments, it may be desirable to administer a composition of the disclosure locally to the area in need of treatment (e.g., to the site of a tumor, such as by injection into the tumor). Other methods of delivery via the hepatic portal vein are also contemplated.

In certain embodiments, compositions of the disclosure are administered by intravenous infusion. In certain embodiments, the a composition is infused over a period of at least 10, at least 15, at least 20, or at least 30 minutes. In other embodiments, the binding element is infused over a period of at least 60, 90, or 120 minutes. Regardless of the infusion period, the disclosure contemplates that, in certain embodiments, each infusion is part of an overall treatment plan where binding element is administered according to a regular schedule (e.g., weekly, monthly, etc.) for some period of time. However, in other embodiments, a composition is delivered by bolus injection, e.g., as part of an overall treatment plan where binding element is administered according to a regular schedule for some period of time.

For any of the foregoing, it is contemplated that compositions of the disclosure (include one binding element or a combination of two or more such binding elements) may be administered in vitro or in vivo via any suitable route or method. Compositions may be administered as part of a therapeutic regimen where a composition is administered one time or multiple times, including according to a particular schedule. Moreover, it is contemplated that the compositions of the disclosure will be formulated as appropriate for the route of administration and particular application. The disclosure contemplates any combination of the foregoing features, as well as combinations with any of the aspects and embodiments of the disclosure described herein.

In some embodiments, the foregoing applies to any compositions (e.g., a particle or plurality of particles) of the disclosure, used alone or in combination, and used for any of the methods described herein. The disclosure specifically contemplates any combination of the features of such compositions of the disclosure, compositions, and methods with the features described for the various pharmaceutical compositions and routes of administration described in this section and below.

In some embodiments, Target can be isolated from subject blood either intracorporeally or extracorporeally by binding with particles or a plurality of particles. In some embodiments, this invention encompasses any and all particles and plurality of particles isolating protein selected from the Targets from blood or other liquid and methods of its use for treatment, including but not limited to anti-aging treatment.

There are several devices, compositions and methods are known in the art that after the modification are applicable for reducing or isolating Target from blood, e.g. to inhibit its biological activity. For example, in some embodiments, this disclosure provides a particle, or a plurality of particles, having a surface comprising a binding element or other agent (e.g., immobilized on a surface of the particle) that binds or selectively binds or catches in any other way a protein selected from Targets. Such agent could be one or more described in this disclosure or some other agent or object binding or catching at least one of Targets. After the Target is bound or caught by the binding element or other agent, it is sequestered by the particle such that the Target has a reduced ability, for example substantially reduced ability or no ability to interact with other molecules or cells or other object it naturally interacted before such binding, so it becomes less active or not active at all. In some embodiments, the implementation of such particles mimics or have comparable therapeutic/anti-aging effect as a use of protein binding or filtering device of this invention.

In some embodiments, this invention encompasses particles comprising reactive groups configured to link a binding element or any other Target-specific agent. Different agents may be attached to a particle based on the Target to be isolated.

In some aspects, the disclosure relates to a kit comprising particles and linkers. For example, linkers may be selected to react with an agent comprising a specific moiety to the particles. In some embodiments, these particles or a plurality of particles have the surface comprising an binding agent that selectively binds to a protein selected from the group of Targets. In some embodiments, these particles or a plurality of particles have reactive groups configured to link an agent to the particles. In some embodiments, these particles or a plurality of particles relate to a kit comprising particles and linkers.

In some embodiments, this invention is a particle having at least one surface and a binding element immobilized on the surface, wherein: the binding element binds or, optionally selectively binds a protein, selected from the group of B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In some embodiments, this invention is a particle comprising a housing, wherein the housing and a binding element immobilized in such housing, wherein: the binding element binds or, optionally selectively binds a protein, selected from the group of B2M, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In some embodiments, this invention is a particle, wherein particle comprises a housing, wherein the housing defines a lumen and the housing further comprises an inlet port for receiving fluid or other substance to the lumen, and an outlet port for releasing fluid or other substance from the lumen, wherein the lumen comprises binding elements, wherein the binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In some embodiments, particle of this invention is designed for circulation in bloodstream of the subject. In some embodiments, particle of this invention is designed to be injectable by into bloodstream of the subject. In some embodiments, such binding inhibits the interaction of such protein with the at least one other molecule or cell it was able to bind to before binding by the binding element immobilized on the particle. In some embodiments, such by agent or nanoparticle in this invention the particle described thereof is meant. Non-limiting examples of methods for manufacturing of particles are suggested in Example 2.

In certain embodiments, the disclosure provides a pharmaceutical composition comprising a molecule or other agent that binds to, inhibits or degrades a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB; and, at least one pharmaceutically acceptable excipient. In some embodiments, the agent binds to, modulates, or degrades a protein. In some embodiments, the agent inhibits or degrades the protein. In some embodiments, the agent is the active ingredient of a pharmaceutical formulation. In some embodiments, the agent is not bound to a stationary phase or platform. In some embodiments, the agent binds to, modulates, inhibits, activates, or degrades a secondary protein or other effector upstream or downstream of a Target, which has an upstream or downstream effect on a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB. In some embodiments, the agent is a binding element.

In certain embodiments, the disclosure provides a pharmaceutical composition comprising a blood plasma fraction, wherein the blood plasma fraction comprises two or more biomarkers characteristic of an aged subject, and wherein the blood plasma fraction comprises a negligible amount of a protein selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In certain aspects, the disclosure provides pharmaceutical compositions of blood plasma fractions enriched or depleted in one or more proteins, e.g., proteins selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB. In certain embodiments, the blood plasma fractions enriched or depleted in a protein comprise two or more biomarkers characteristic of an aged subject. In certain embodiments, a pharmaceutical composition of the disclosure comprises a blood plasma fraction comprising two or more biomarkers characteristic of an aged subject and a negligible amount of one or more proteins selected from B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In some embodiments, a negligible amount, as used in reference to the amount of a protein in a pharmaceutical composition, is less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml. In some embodiments, a negligible amount is less than 1 ng/ml, less than 0.80 ng/mL, less than 0.50 ng/mL, less than 0.25 ng/ml, less than 0.12 ng/ml, less than 0.06 ng/ml, less than 0.04 ng/ml, less than 0.02 ng/ml, less than 0.01 ng/ml, or less than 0.005 ng/ml, less than 0.004 ng/ml, less than 0.003 ng/ml, less than 0.002 ng/ml, less than 0.001 ng/ml, less than 0.0007 ng/ml, less than 0.0005 ng/ml, less than 0.0003 ng/ml, less than 0.0002 ng/ml, less than 0.0001 ng/ml, less than 0.08 pg/ml, less than 0.06 pg/ml, less than 0.04 pg/ml, less than 0.02 pg/ml or less than 0.01 pg/ml.

In some embodiments, a negligible amount is between about 0.001 ng/ml to about 15,000 ng/ml. In some embodiments, a negligible amount is between about 0.0005 ng/ml to about 15,000 ng/ml. In some embodiments, a negligible amount is between about 0.001 ng/ml to about 15,000 ng/ml. In some embodiments, a negligible amount is between about 0.08 pg/ml to about 15,000 ng/ml. In some embodiments, a negligible amount is between about 0.02 pg/ml to about 15,000 ng/ml. In some embodiments, a negligible amount is between about 0.01 pg/ml to about 15,000 ng/ml. In some embodiments, a negligible amount is between about 0.01 pg/ml to about 0.1 pg/ml.

In some embodiments, a negligible amount is between about 0.01 pg/ml to about 0.05 pg/ml. In some embodiments, a negligible amount is between about 0.5 pg/ml to about 1 pg/ml. In some embodiments, a negligible amount is between about 0.001 pg/ml to about 0.01 pg/ml.

In some embodiments, a negligible amount is between about 0.005 ng/ml to about 15,000 ng/ml. In some embodiments, a negligible amount is between about 0.01 ng/ml to about 15,000 ng/ml. In some embodiments, a negligible amount is between about 0.02 ng/ml to about 15,000 ng/ml. In some embodiments, a negligible amount is between about 0.04 ng/ml to about 15,000 ng/ml. In some embodiments, a negligible amount is between about 0.06 ng/ml to about 15,000 ng/ml. In some embodiments, a negligible amount is between about 0.12 ng/ml to about 15,000 ng/ml. In some embodiments, a negligible amount is between about 0.25 ng/ml to about 15,000 ng/ml. In some embodiments, a negligible amount is between about 0.5 ng/ml to about 15,000 ng/ml. In some embodiments, a negligible amount is between about 1.0 ng/ml to about 10,000 ng/ml. In some embodiments, a negligible amount is between about 5 ng/ml to about 10,000 ng/ml. In some embodiments, a negligible amount is between about 10 ng/ml to about 10,000 ng/ml. In some embodiments, a negligible amount is between about 25 ng/ml to about 10,000 ng/ml. In some embodiments, a negligible amount is between about 50 ng/ml to about 10,000 ng/ml. In some embodiments, a negligible amount is between about 100 ng/ml to about 5,000 ng/ml. In some embodiments, a negligible amount is between about 200 ng/ml to about 2,000 ng/ml. In some embodiments, a negligible amount is between about 300 ng/ml to about 1,000 ng/ml. In some embodiments, a negligible amount is between about 500 ng/ml to about 1,000 ng/ml. In some embodiments, a negligible amount, as used in reference to the amount of a protein in a pharmaceutical composition, is used in the meaning “decreased” level or amount compared to a control/reference level/average physiological level/level in subject prior to taken the sample. In some embodiments, a negligible amount as described above, are used in reference to the amount of a protein in a blood.

In some embodiments are pharmaceutical compositions comprising a blood plasma fraction, wherein the blood plasma fraction comprises a negligible amount of at least two proteins selected from B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In some embodiments, this disclosure provides a medication, which is a molecule or nanoparticle, other particle or any other agent that binds to, modulates, inhibits, or degrades a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB or at least one effector upstream or downstream of at least one of the Targets. In some embodiments binding or degradation or modulation or inhibition or activation of such at least one effector upstream or downstream of at least one of the Targets has anti-aging effect, thus such agent and the use of respective binding element or degradating agent or modulator or inhibitor as an anti-aging medication is also covered by this disclosure. In some embodiments, such molecule or nanoparticle or other particle or other agent is for use as an anti-aging medication or for use for the treatment of aging or rejuvenation. In some embodiments, such agent is selected from the following group: protein, aptamer, peptide, polymer, virus, small molecule, wherein such agent selectively binds or degrades at least one protein selected from the Targets. In some embodiments such molecule selectively bind at least one of the Targets with a K_{D} of less than 1×10⁻⁴ M, with a K_{D} of less than 1×10⁻⁵ M, with a K_{D} of less than 1×10⁻⁶ M, with a K_{D} of less than 1×10⁻⁷ M, with a K_{D} of less than 1×10⁻⁸ M, with a K_{D} of less than 1×10⁻⁹ M.

In some embodiments, this invention is a human monoclonal antibody, or a Target binding portion thereof or composition comprising human monoclonal antibodies or a Target binding portion thereof that bind specifically to a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB or antibody or protein binding portion thereof binding at least one effector upstream or downstream of at least one of the Targets. In some embodiments, this invention is a pharmaceutical composition comprising at least one pharmaceutically acceptable excipient and antibody selected from the group: human anti-B2M mAb, human anti-CCDC80 mAb, human anti-CD59 mAb, human anti-CHRDL1 mAb, human anti-COL18A1 mAb, human anti-CST3 mAb, human anti-DPT mAb, human anti-EFEMP1 mAb, human anti-FAS mAb, human anti-FSTL3 mAb, human anti-GAS1 mAb, human anti-GDF15 mAb, human anti-KLK11 mAb, human anti-MMP7 mAb, human anti-NBL1 mAb, human anti-NTN1 mAb, human anti-POSTN mAb, human anti-PTN mAb, human anti-RELT mAb, human anti-SFRP1 mAb, human anti-SMOC1 mAb, human anti-STC1 mAb, human anti-TNFRSF1A mAb, human anti-UNC5C mAb, human anti-sFRP-3 mAb, human anti-TNFRSF1B mAb, human anti-CD55 mAb, human anti-BMP4 mAb and human anti-RGMB mAb.

In some embodiments, this invention is a pharmaceutical composition comprising at least one pharmaceutically acceptable excipient and antibody selected from the group: humanized anti-CCDC80 mAb, humanized anti-CD59 mAb, humanized anti-CHRDL1 mAb, humanized anti-COL18A1 mAb, humanized anti-CST3 mAb, humanized anti-DPT mAb, humanized anti-EFEMP1 mAb, humanized anti-FAS mAb, humanized anti-FSTL3 mAb, humanized anti-GAS1 mAb, humanized anti-GDF15 mAb, humanized anti-KLK11 mAb, humanized anti-MMP7 mAb, humanized anti-NBL1 mAb, humanized anti-NTN1 mAb, humanized anti-POSTN mAb, humanized anti-PTN mAb, humanized anti-RELT mAb, humanized anti-SFRP1 mAb, humanized anti-SMOC1 mAb, humanized anti-STC1 mAb, humanized anti-TNFRSF1A mAb, humanized anti-UNC5C mAb, humanized anti-sFRP-3 mAb, humanized anti-TNFRSF1B mAb, humanized anti-CD55 mAb, humanized anti-BMP4 mAb and humanized anti-RGMB mAb.

In some embodiments, this invention is a pharmaceutical composition comprising at least one pharmaceutically acceptable excipient and antibody selected from the group: chimeric anti-CCDC80 mAb, chimeric anti-CD59 mAb, chimeric anti-CHRDL1 mAb, chimeric anti-COL18A1 mAb, chimeric anti-CST3 mAb, chimeric anti-DPT mAb, chimeric anti-EFEMP1 mAb, chimeric anti-FAS mAb, chimeric anti-FSTL3 mAb, chimeric anti-GAS1 mAb, chimeric anti-GDF15 mAb, chimeric anti-KLK11 mAb, chimeric anti-MMP7 mAb, chimeric anti-NBL1 mAb, chimeric anti-NTN1 mAb, chimeric anti-POSTN mAb, chimeric anti-PTN mAb, chimeric anti-RELT mAb, chimeric anti-SFRP1 mAb, chimeric anti-SMOC1 mAb, chimeric anti-STC1 mAb, chimeric anti-TNFRSF1A mAb, chimeric anti-UNC5C mAb, chimeric anti-sFRP-3 mAb, chimeric anti-TNFRSF1B mAb, chimeric anti-CD55 mAb, chimeric anti-BMP4 mAb and chimeric anti-RGMB mAb.

In some embodiments such human monoclonal antibody or composition comprising human monoclonal antibodies is for use as anti-aging treatment. In some embodiments such human monoclonal antibody is for use as binding element for protein binding device. In some embodiments such human monoclonal antibody is isolated antibody. In some embodiments such antibody selectively binds at least one of the Targets with a K_{D} of less than 1×10⁻⁴ M, with a K_{D} of less than 1×10⁻⁵ M, with a K_{D} of less than 1×10⁻⁶ M, with a K_{D} of less than 1×10⁻⁷ M, with a K_{D} of less than 1×10⁻⁸ M, with a K_{D} of less than 1×10⁻⁹ M. In some embodiments this invention is an isolated human antibody, or an antigen binding portion thereof that dissociates from human protein, selected from the Targets with a K_{D} of less than 1×10-4 M, with a K_{D} of less than 1×10-5 M, with a K_{D} of less than 1×10-6 M, with a K_{D} of less than 1×10-7 M, with a K_{D} of less than 1×10-8 M, with a K_{D} of less than 1×10-9 M, determined by surface plasmon resonance.

In some embodiments this invention is an aqueous composition consisting of a solvent which consists at least partly of water, an anti-Target antibody, a buffer, an amino acid, a surfactant, and sodium chloride as isotonic agent.

In some embodiments, this disclosure provides a medication or pharmaceutical composition comprising binding element described in this disclosure or its structural or functional analog or its prodrug, solvate, tautomer or stereoisomer thereof as well as the physiologically acceptable salts of each of the foregoing, including mixtures thereof in all ratios.

In some embodiments, this disclosure provides pharmaceutical compositions comprising an agent that binds to, modulates, inhibits or degrades a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB; and, at least one pharmaceutically acceptable excipient, wherein the agent is described in this disclosure, including but not limited to agents described as binding element in this application or its structural or functional or SAR analog or prodrug. In some embodiments this invention is a an antibody or any other molecule or particle having at least 75%, 80%, 85%, 90%, 95%, 99% sequence similarity to at least one of the antibodies described in this disclosure as binding element or with the Target binding portion thereof, or pharmaceutical composition comprising such antibody or Target binding portion thereof, optionally for use as medication, including but not limited to anti-aging treatment.

In some embodiments, the antibodies and other binding elements described in this application or alike molecules should be requested for delivery in the form suitable for injection into animal or human subject, e.g. free of endotoxins, azides and other elements or objects not suitable for animal and human administration. In case the such binding elements are delivered with such elements or objects the binding element should be purified from such objects by the method known in the art, e.g. described in GE Healthcare Antibody Purification Handbook 18-1037-46 AD or by rules applicable for medications production or for medication used in clinical trials.

In some embodiments such pharmaceutical composition is for use as an anti-aging medication or for use for the anti-aging treatment.

In some embodiment this invention is a molecule or other agent obtained by the in-vitro or in-silico or ex-vivo screening for binding or inhibition or degradation or deactivation of at least one of the Targets or protein binding device comprising such molecule. In some embodiment this invention is a protein binding device, wherein binding element is obtained by the in-vitro or in-silico or ex-vivo screening for binding or inhibition or degradation or deactivation of at least one of the Targets or protein binding device comprising such molecule.

Non-limiting examples of plasma obtained by degradation of proteins selected from Targets are shown in Example 30.

In some instances, the pharmaceutical composition described herein is formulated for intravenous administration. Compositions for intravenous administration can comprise a sterile isotonic aqueous buffer. The compositions can also include a solubilizing agent. Compositions for intravenous administration can optionally include a local anesthetic such as lignocaine to lessen pain at the site of the injection. Where the pharmaceutical composition described herein is administered by infusion, it can be dispensed, for example, with an infusion bottle containing sterile pharmaceutical grade water or saline. Where the pharmaceutical composition described herein is administered by injection, an ampule of sterile water for injection or saline can be provided so that the enzyme or enzyme and antioxidant and the carrier can be mixed prior to administration. One of the many possible forms of this invention can be a Lyophilized Concentrate for Intravenous (IV) Injection.

Non-limiting examples of pharmaceutical compositions of this disclosure are shown in examples 29 and 30.

The amount of pharmaceutical composition described herein that is effective for treating a corresponding disease or condition can be determined using standard clinical or pharmacokinetic techniques known to those with skill in the art. In addition, in vitro or in vivo assays can optionally be employed to help identify optimal dosage ranges. The precise dose to be employed can also depend on the route of administration, the disease or condition, the seriousness of the corresponding disease or condition being treated, as well as various physical factors related to the individual being treated, and can be decided according to the judgment of a health-care practitioner. For example, any agent or composition of this disclosure, in an amount ranging from about 0.05 pg/kg to about 100 mg/kg of a patient's body weight or 0.01 to about 1000 mg/kg of total body weight per day, or from about 0.1 to about 100 mg/kg of total body weight per day, or from about 0.5 to about 15 mg/kg of total body weight per day, or from about 1 mg/kg to about 50 mg/kg of total body weight, which may be administered in one or multiple doses per day or per week or per month or per 6 months or per year or per 3 years or per 8 years or per 12 years or once in a lifetime. Equivalent dosages can be administered over various time periods including, but not limited to, about every 2 hours, about every 4 hours, about every 8 hours, about every 12 hours, about every 24 hours, about every 36 hours, about every 48 hours, about every 72 hours, about every week, about every two weeks, about every three weeks, about every month, and about every two months or every 6 months or every year or every 3 years or every 8 years or every 12 years or once in a lifetime or by periods lifelong as decided by practitioner or patient. The number and frequency of dosages corresponding to a completed course of therapy can be determined according to the judgment of a health-care practitioner.

In some embodiments, the pharmaceutical composition and formulations described herein are administered to a subject by any suitable administration route, including but not limited to, parenteral (e.g., intravenous, subcutaneous, intramuscular), intradermal, intraperitoneal, subcutaneous, intranasal, epidural, sublingual, intravaginal, rectal, by inhalation, topical intracerebral, oral, intranasal, buccal, rectal, or transdermal administration routes. For example, in some instances, the pharmaceutical composition described herein is administered locally. This is achieved, for example, by local infusion during surgery, by injection, by means of a catheter, by means of a suppository or enema, or by means of an implant, the implant being of a porous, non-porous, or gelatinous material, including membranes, such as sialastic membranes, or fibers. In some situations, the pharmaceutical composition described herein is introduced into the central nervous system, circulatory system or gastrointestinal tract by any suitable route, including intraventricular, intrathecal injection, paraspinal injection, epidural injection, enema, and by injection adjacent to a peripheral nerve. Pulmonary administration can also be employed, e.g., by use of an inhaler or nebulizer, and formulation with an aerosolizing agent, or via perfusion in a fluorocarbon or synthetic pulmonary surfactant.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binding elements, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrolidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pa. 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some embodiments, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some embodiments, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some embodiments, the pharmaceutical formulations further include diluents which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some embodiments, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethycellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some embodiments, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil@, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or Tween® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Methods and Uses

In certain embodiments, the disclosure provides methods for detecting the presence and/or concentration of proteins in the plasma of a subject in need thereof, comprising introducing plasma to a protein binding device, such as a protein binding device described herein, and detecting the presence or amount of protein bound to the first group of binding elements and/or second group of binding elements. In certain embodiments, the protein is selected from the group: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB.

In certain embodiments, the disclosure provides methods for reducing the concentration of a protein in the plasma of a subject in need thereof, comprising introducing the plasma to a protein binding device, such as a protein binding device described herein, wherein the plasma enters the inlet port and contacts the binding elements and is discharged from the outlet port as modified plasma, and retrieving the modified plasma from the protein binding device wherein the concentration of the protein in the modified plasma is reduced relative to the concentration of the protein in the plasma introduced to the protein binding device. The protein may be selected from the group: CCDC80, CD59, CHRDL1, COL8A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB.

In certain embodiments, the disclosure provides methods for reducing the concentration of proteins in the plasma of a subject in need thereof, comprising introducing the plasma to a protein binding device, such as a protein binding device described herein, wherein the plasma contacts the first group and second group of binding elements and is discharged from the protein binding device as modified plasma, and retrieving the modified plasma from the protein binding device wherein the concentration of the proteins in the modified plasma is reduced relative to the concentration of the proteins in the plasma introduced to the protein binding device. The protein may be selected from the group: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB.

In certain embodiments, the disclosure provides methods for reducing the concentration of a protein in the plasma of a subject in need thereof, comprising introducing the plasma to a plasma filtration device, such as a plasma filtration device described herein, wherein the plasma is introduced to the lumen of the housing and contacts the filter element and is discharged from the plasma filtration device as modified plasma, and retrieving the modified plasma from the plasma filtration device wherein the concentration of the protein in the modified plasma is reduced relative to the concentration of the protein in the plasma introduced to the plasma filtration device. The protein may be selected from the group: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB.

In certain embodiments, the disclosure provides method for reducing the concentration of a protein in plasma, comprising contacting plasma with protein binding elements that selectively bind a protein selected from: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, and, optionally, collecting modified plasma following the contacting step, wherein the modified plasma has a reduced concentration of the protein relative to the concentration of the protein in the plasma prior to the contacting. In some embodiments, the protein is reduced by about 10% or more, about 20% or more, about 30% or more, about 40% or more, about 50% or more, about 60% or more, or about 70% or more, or about 90% or more, or about 95% or more, or about 99% or more, or about 99.9% or more. In some embodiments, the protein is reduced by between about 1% to about 99%. In some embodiments, the protein is reduced by between about 1% to about 90%. In some embodiments, the protein is reduced by between about 1% to about 50%. In some embodiments, the protein is reduced by between about 1% to about 20%. In some embodiments, the protein is reduced by between about 10% to about 80%. In some embodiments, the protein is reduced by between about 10% to about 60%. In some embodiments, the protein is reduced by between about 10% to about 40%. In some embodiments, the protein is reduced by between about 10% to about 20%. In some embodiments, the protein is reduced by between about 20% to about 90%. In some embodiments, the protein is reduced by between about 30% to about 80%. In some embodiments, the protein is reduced by between about 40% to about 70%. In some embodiments, the protein is reduced by between about 40% to about 60%. In some embodiments, the protein is reduced by between about 90% to about 99.99%. In some embodiments, the protein is reduced by between about 70% to about 99.99%. In some embodiments, the protein is reduced by between about 90% to about 99%. In some embodiments, the protein is reduced by between about 99% to about 99.99%. In some embodiments, the protein is reduced by between about 99.9% to about 99.99%. In some embodiments, the protein is reduced by between about 99% to about 99.999%.

For the references, some of the concentrations of Targets in plasma known in the art, marked in grey are reported concentrations which are outliers related to the other sources.

| | | | | Blood plasma | | |
|---|---|---|---|---|---|---|
| | | | | conc (Plasma | | |
| | | | | proteome | | |
| | | | | database | | |
| | | | | http:// | Olink | |
| | Name of | | | www.plasmaproteome | range(https:// | |
| | target | | UniProt | database.org) | www.olink.com) | Literature, |
| | protein | Protein full name | number | ng/ml | ng/ml | ng/ml |
| 1 | B2M | Beta-2-microglobulin | P61769 | 1100-7500 | — | 100-2000 |
| | | | | | | (less than |
| | | | | | | 300 is a |
| | | | | | | norm, >300 |
| | | | | | | is a risk) |
| 2 | CCDC80 | Coiled-coil domain- | Q76M96 | 12 | ~1 | 0.5-4 |
| | | containing protein 80 | | | | |
| 3 | CD59 | CD59 glycoprotein | P13987 | 33-120, 910 | 0.01-0.05 | 33-143, 200 |
| 4 | CHRDL1 | Chordin-like protein 1 | Q9BU40 | 28 | — | — |
| 5 | COL18A1 | Endostatin | P39060 | 35 | ~0.05 | 2-7, 70-300, |
| | | | | | | 30-60 |
| 6 | CST3 | Cystatin-C | P01034 | 200-1100 | ~1 | 800-1100 |
| 7 | DPT | Dermatopontin | Q07507 | — | — | — |
| 8 | EFEMP1 | Fibulin-3, EGF- | Q12805 | 260 | ~1 | 14-114 |
| | | containing fibulin-like | | | | |
| | | extracellular matr . . . | | | | |
| 9 | FAS | Tumor necrosis factor | P25445 | — | ~10 | 0.5-6 |
| | | receptor superfamily | | | | |
| | | mem . . . | | | | |
| 10 | FSTL3 | Follistatin-related | O95633 | 12 | ~1 | 20-30, 5-8 |
| | | protein 3 | | | | |
| 11 | GAS1 | Growth arrest-specific | P54826 | — | — | 0.7-1 (rat |
| | | protein 1 | | | | plasma) |
| 12 | GDF15 | Growth/differentiation | Q99988 | 1.5 +/− 0.69 | ~1 | 0.5-1, 0.5- |
| | | factor 15 | | | | 10, 0.5-5 |
| 13 | KLK11 | Kallikrein-11 | Q9UBX7 | 2200 | — | 300-10000, |
| | | | | | | 4 |
| 14 | MMP7 | Matrilysin | P09237 | 0.04-0.07 | ~1 | 5-9, 10-20, |
| | | | | | | 1.7-30, 0.2 |
| 15 | NBL1 | Neuroblastoma | P41271 | 70 | ~7 | — |
| | | suppressor of | | | | |
| | | tumorigenicity 1 | | | | |
| 16 | NTN1 | Netrin-1 | O95631 | — | — | 0.02-0.15, |
| | | | | | | 0.1-1 |
| 17 | POSTN | Periostin | Q15063 | 16 | — | 140-250, |
| | | | | | | 20-50, 40- |
| | | | | | | 60, 20-100 |
| 18 | PTN | Pleiotrophin | P21246 | — | ~1 | 300-500, |
| | | | | | | 20-30 |
| 19 | RELT | Tumor necrosis factor | Q969Z4 | — | 0.05-0.1 | — |
| | | receptor superfamily | | | | |
| | | mem . . . | | | | |
| 20 | SFRP1 | Secreted frizzled- | Q8N474 | — | — | 0.07-0.11 |
| | | related protein 1 | | | | |
| 21 | SMOC1 | SPARC-related | Q9H4F8 | — | — | — |
| | | modular calcium- | | | | |
| | | binding protein 1 | | | | |
| 22 | STC1 | Stanniocalcin-1 | P52823 | — | 5-100 | 0.2-1.5 |
| 23 | TNFRSF1A | Tumor necrosis factor | P19438 | 0.42-5.75 | ~10 | 0.7-1.1, 0.7- |
| | | receptor superfamily | | | | 2, 0.2-13 |
| | | mem . . . | | | | |
| 24 | UNC5C | Netrin receptor | O95185 | — | ~10 | — |
| | | UNC5C | | | | |
| 25 | sFRP-3 | Secreted frizzled- | Q92765 | — | ~1 | 1000-10000 |
| | (FRZB) | related protein 3 | | | | |
| 26 | TNFRSF1B | | P20333 | 0.42-22 | ~1 | 0.6-31 |
| 27 | RGMB | | | — | 50 | — |
| 28 | CD55 | | P08174 | — | — | — |
| 29 | BMP4 | | P12644 | — | 0.1-1 | 0.1-0.15, |
| | | | | | | 0.003- |
| | | | | | | 0.006, |
| | | | | | | 0.01-0.02 |

There are median levels of Target protein in 50+ age in women in Relative fluorescence intensities (RFU) as more specifically explained in Somalogic documentation B2M-1920 RFU, CCDC80-2753, CD59-1800, CHRDL1-3377, COL18A1 82157, CST3-2244, DPT-4332, EFEMP1-1767, FAS-2060, FSTL3-8609, GAS1-1151, GDF15 1672, KLK11-2964, MMP7-2343, NBL1-5212, NTN1-844, POSTN-3459, PTN-1652, RELT-1771, SFRP1-2393, SMOC1-4935, STC1-2526, TNFRSF1A-1991, UNC5C-4820, FRZB-4109 TNFRSF1B-18797, CD55-19906, RGMB-2412.

In some embodiments, protein CCDC80 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml. In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein CD59 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml. In some embodiments, protein CHRDL1 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein COL8A1 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein CST3 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein DPT is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml.

In some embodiments, protein EFEMP1 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein FAS is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein FSTL3 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein GAS1 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein GDF15 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein KLK11 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein MMP7 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein NBL1 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml. In some embodiments, protein NTN1 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein POSTN is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein PTN is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein RELT is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein SFRP1 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein SMOC1 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein STC1 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein TNFRSF1A is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein UNC5C is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein sFRP-3 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein TNFRSF1B is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein CD55 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein BMP4 is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

In some embodiments, protein RGMB is reduced to less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml, less than 0.01 pg/ml, less than 0.008 pg/ml, less than 0.006 pg/ml, less than 0.004 pg/ml, less than 0.002 pg/ml, less than 0.001 pg/ml. In some embodiments, the protein is reduced to between about 0.005 ng/ml and about 15,000 ng/ml. In some embodiments, the protein is reduced to between about 1.0 ng/ml and about 10,000 ng/ml. In some embodiments, the protein is reduced to between about 100 ng/ml and about 5,000 ng/ml. In some embodiments, the protein is reduced to between about 200 ng/ml and about 2,000 ng/ml. In some embodiments the protein is reduced to between about 300 ng/ml and about 1,000 ng/ml. In some embodiments, the protein is reduced to between about 500 ng/ml and about 1,000 ng/ml, In some embodiments, the protein is reduced to between about 0.001 ng/ml and about 15,000 ng/ml. In some embodiments, the protein is reduced to between about 0.0005 ng/ml and about 15,000 ng/ml. In some embodiments, the protein is reduced to between about 0.001 ng/ml and about 15,000 ng/ml. In some embodiments, the protein is reduced to between about 0.08 pg/ml and about 15,000 ng/ml. In some embodiments, the protein is reduced to between about 0.02 pg/ml and about 15,000 ng/ml. In some embodiments, the protein is reduced to between about 0.01 pg/ml and about 15,000 ng/ml. In some embodiments, the protein is reduced to between about 0.01 pg/ml and about 0.1 pg/ml. In some embodiments, the protein is reduced to between about 0.01 pg/ml and about 0.05 pg/ml. In some embodiments, the protein is reduced to between about 0.5 pg/ml and about 1 pg/ml.

It is envisaged herein that the level, amount or concentration of a protein selected from the group CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB is reduced by at least 10% compared to the initial level, amount or concentration of said protein, preferably by at least 20%, 30%, 40%, 50%, 60%, 70%, 80%, 90%, 95%, 96%, 97%, 98%, 99% or 100%. If the level, amount or concentration of the protein is reduced by 100% compared to the initial level it is no longer present or is only present in trace amounts.

In some embodiments, the method further comprises separating plasma from whole blood prior to the contacting.

In some embodiments, the protein binding element is associated with a protein binding column. In some embodiments, the contacting comprises passing the blood plasma fraction through a protein binding column.

Described herein are pharmaceutical compositions comprising the modified plasma described herein.

Described herein are methods of treating or preventing an age-related disease or disorder or other anti-aging treatment comprising administering to a subject in need thereof a pharmaceutical composition described herein.

Described herein are methods of treating or preventing an age-related disease or disorder or other anti-aging treatment comprising administering to a subject in need thereof a binding element, a modulator, of a protein selected from B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB or at least one effector upstream or downstream of at least one of such proteins.

In some embodiments, the modulator is a small molecule inhibitor.

In some embodiments, the modulator affects the target protein or mimics the effect of Target inhibition, degradation or reduction by contacting at least one effector upstream or downstream of at least one of the Targets.

In some embodiments, an age related disease or disorder is selected from: atherosclerosis, cardiovascular disease, adult cancer, arthritis, cataracts, osteoporosis, type 2 diabetes, hypertension, neurodegenaration, including but not limited to Alzheimer's disease, dementia, Parkinson's disease), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, myocardial infarction, acute coronary syndrome, sarcopenia, sarcopenic obesity, senile osteoporosis, urinary incontinence or diseases and conditions mentioned in “Frameworks for Proof-of-Concept Clinical Trials of Interventions That Target Fundamental Aging Processes”. Justice et al., 2016), Juvenescence: Investing in the Age of Longevity, Mellon at al., 2017) etc.

Aging or age related change in any parameter of organism is also regarded as an aging related condition, including but not limited to aging related change in blood parameters, heart rate, cognitive functions/decline, bone density, basal metabolic rate, systolic blood pressure, heel bone mineral density (BMD), heel quantitative ultrasound index (QUI), heel broadband ultrasound attenuation, heel broadband ultrasound attenuation, forced expiratory volume in 1-second (FEV1), forced vital capacity (FVC), peak expiratory flow (PEF), duration to first press of snap-button in each round, mean time to correctly identify matches, hand grip strength (right and left), whole body fat-free mass, leg fat-free mass (right and left), and time for recovery after the stress (wound, operation, chemotherapy, disease, change in lifestyle etc.). Aging related change in any parameter, including but not limited to selected from the Table “Declines” is also regarded as aging related condition. In some embodiments, age related, aging-related or ageing-related means “caused by pathological processes which persistently lead to the loss of organism's adaptation and progress in older ages”.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

In certain embodiments, the disclosure provides a protein binding device or in some other embodiments a fluid filtration device for the selective binding, modulation, reduction or degradation of one or more proteins.

In some embodiments, the term “biomarker” as used herein, refers to any molecule that exerts an effect on a living organism. In some embodiments, a biomarker is a molecule that is created in an organism. In some embodiments, a biomarker is an atom, such as lithium or lead (e.g., the biomarker may be a metal cation). In some embodiments, the biomarker is not an atom or metal ion. For example, the biomarker may be a molecule, such as an organic compound or inorganic compound. In some embodiments, the biomarker is a drug. In some embodiments, the biomarker is a psychoactive drug, such as diacetylmorphine. In some embodiments, a biomarker is a poison, toxin, or venom. In some embodiments, a biomarker is a molecule that is created in an organism a biomarker is an allergen. In some embodiments, a biomarker is a carcinogen. In some embodiments, a biomarker is a molecule that is endogenous to the organism, such as a hormone, cytokine, neurotransmitter, soluble extracellular receptor, antibody, or soluble matrix protein. In some embodiments, a biomarker is a peptide, polypeptide, protein, nucleic acid, carbohydrate, or sugar. In some embodiments, a biomarker comprises a peptide, polypeptide, protein, nucleic acid, carbohydrate, or sugar. In some embodiments, a biomarker is a misfolded protein. In some embodiments, a biomarker is an amyloid or the soluble precursor of an amyloid. “Polypeptide,” “peptide,” and “protein” are used interchangeably and mean any peptide-linked chain of amino acids, regardless of length or post-translational modification. In some embodiments, a biomarker is a lipid, a steroid, or cholesterol. In some embodiments, a biomarker comprises alipid, a steroid, or cholesterol. In some embodiments, a biomarker is a circulating, cell-free nucleic acid, such as a circulating, cell-free RNA. In some embodiments, a biomarker is a micro RNA (miRNA).

In some embodiments, a biomarker is a molecule that is secreted by a cell (e.g., a mammalian cell). In some embodiments, a biomarker is an extracellular region of a membrane protein that is susceptible to cleavage into a soluble form. In some embodiments, a biomarker is a cytosolic biomolecule. For example, the biomarker may be a cytosolic biomolecule that is released in vivo following apoptosis, or a binding element may be used in an in vitro method in which the cytosolic biomolecule is free in solution.

In some embodiments, a biomarkers concentration is linked to the presence of a protein in a body. In some embodiments, the removal, binding, or sequestration of a target protein produces a change in the concentration of one or more biomarkers. In some embodiments, the protein is soluble in aqueous solution or blood. In some embodiments, the binding element binds a soluble protein. In some embodiments, the binding element binds an insoluble protein. For example, in some embodiments, a binding element specifically binds a protein that is associated with a protein aggregate, such as amyloid or a prion aggregate. Such binding provides a therapeutic benefit by disassembling the aggregate (e.g., by shifting a thermodynamic equilibrium away from aggregated states) and/or by sequestering the aggregate (e.g., to inhibit further aggregation and/or to allow for clearance of the bound aggregate). In some embodiments, a binding element specifically binds to a protein that is associated with a virus or cell, such as a bacterial, protozoan, fungal, or yeast cell, e.g., wherein the protein is not a solute in aqueous solution, but the protein is partitioned into a membrane, cell wall, or capsid. Thus, a binding element is able to sequester a pathogenic virus or cell, thereby attenuating the pathogenicity of the virus or cell. In some embodiments, a biomarker is a binding element specifically binds to a protein that is associated with an extracellular vesicle, such as an ectosome, exosome, shedding vesicle, or apoptotic body. In some embodiments, a binding element specifically binds to a low-density lipoprotein, e.g., to sequester low-density lipoprotein particles.

Below is non-limiting list of selected biomarkers related to aging and age related diseases. Table 1 (below) shows the selected biomarkers and the average concentration of the biomarkers in human females of varying age from National Health and Nutrition Examination Survey (NHANES). In some embodiments, blood is said to be “aged” or “old” when one or more biomarkers that are present in the blood have a concentration that falls within the average described in the Table 1 for the over 60 column. In some embodiments, blood is said to be “aged” or “old” when one or more biomarkers that are present in the blood have a concentration that falls within the average described in Table 1 for the 41-60 column or the over 61 column. In some embodiments, blood is said to be “young” when one or more biomarkers that are present in the blood have a concentration that falls within the average described in Table 1 for the 20-40 column.

In a preferred aspects the term “biomarker” refers to the biomarkers given in the table below or other aging, morbidity and mortality biomarkers, whereas the term “target” refers to the protein selected from the group: “B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB”

TABLE 1

| | | Average | Average | Average |
|---|---|---|---|---|
| | | concentration | concentration | concentration |
| | | in Human | in Human | in Human |
| Biomarker name | | Females 20-40 | Females 40-60 | Females Over |
| and concentration | Testing Category | Years Old | Years Old | 60 Years Old |
| Glucose, serum | Standard Biochemistry Profile | 86.505 ± 12.528 | 94.907 ± 18.310 | 102.873 ± 24.484 |
| (mg/dL) | | | | |
| Glycohemoglobin | Standard Biochemistry Profile | 5.199 ± 0.411 | 5.600 ± 0.667 | 5.894 ± 0.774 |
| Creatinine (mg/dL) | Standard Biochemistry Profile | 0.676 ± 0.150 | 0.733 ± 0.167 | 0.830 ± 0.250 |
| Lactate | Standard Biochemistry Profile | 121.218 ± 24.189 | 131.855 ± 27.464 | 142.085 ± 29.029 |
| dehydrogenase | | | | |
| LDH (U/L) | | | | |
| Uric acid (mg/dL) | Standard Biochemistry Profile | 4.372 ± 1.058 | 4.754 ± 1.236 | 5.312 ± 1.427 |
| Blood lead (ug/dL) | Blood Lead, Cadmium Total | 0.959 ± 0.613 | 1.504 ± 0.955 | 1.901 ± 1.141 |
| | Mercury, Selenium, and | | | |
| | Manganese | | | |
| Homocysteine(umol/L) | Cadmium, Lead, Mercury, | 5.901 ± 1.991 | 7.625 ± 2.536 | 9.831 ± 3.677 |
| | Cotinine & Nutritional | | | |
| | Biochemistries | | | |
| Vitamin A (ug/dL) | Cadmium, Lead, Mercury, | 47.905 ± 14.635 | 54.876 ± 16.140 | 63.170 ± 18.854 |
| | Cotinine & Nutritional | | | |
| | Biochemistries | | | |
| Fasting Glucose | Plasma Fasting Glucose | 91.314 ± 11.583 | 99.839 ± 16.602 | 108.872 ± 24.438 |
| (mg/dL) | | | | |
| GGT: SI (U/L) | Standard Biochemistry Profile | 16.929 ± 10.166 | 22.610 ± 14.717 | 23.385 ± 14.505 |
| Total cholesterol | Cholesterol - Total | 188.959 ± 41.150 | 204.618 ± 39.774 | 209.085 ± 42.314 |
| (mg/dL) | | | | |
| Vitamin E (ug/dL) | Cadmium, Lead, Mercury, | 1110.766 ± 379.267 | 1304.002 ± 504.653 | 1642.514 ± 709.428 |
| | Cotinine & Nutritional | | | |
| | Biochemistries | | | |
| Chloride: SI | Standard Biochemistry Profile | 104.414 ± 2.515 | 103.808 ± 2.868 | 103.035 ± 3.283 |
| (mmol/L) | | | | |
| AST: SI (U/L) | Standard Biochemistry Profile | 20.971 ± 5.989 | 23.065 ± 7.410 | 24.127 ± 7.369 |
| Sodium: SI | Standard Biochemistry Profile | 138.351 ± 2.068 | 139.017 ± 2.269 | 139.447 ± 2.591 |
| (mmol/L) | | | | |
| PCB180 (ng/g) | Dioxins, Furans, & Coplanar | 0.064 ± 0.053 | 0.203 ± 0.145 | 0.376 ± 0.230 |
| | PCBs | | | |
| Cholesterol (mg/dL) | Standard Biochemistry Profile | 189.532 ± 41.421 | 204.659 ± 40.038 | 208.133 ± 42.947 |
| PCB170 (ng/g) | Dioxins, Furans, & Coplanar | 0.030 ± 0.021 | 0.081 ± 0.059 | 0.143 ± 0.084 |
| | PCBs | | | |
| Alkaline | Standard Biochemistry Profile | 65.141 ± 23.674 | 71.680 ± 23.824 | 76.443 ± 25.038 |
| phosphatase(U/L) | | | | |
| PCB180 Lipid | Dioxins, Furans, & Coplanar | 10.656 ± 8.585 | 32.092 ± 23.241 | 55.014 ± 34.377 |
| Adjusted | PCBs | | | |
| Oxychlordane Lipid | Dioxins, Furans, & Coplanar | 8.084 ± 5.809 | 17.897 ± 11.449 | 36.521 ± 21.944 |
| Adjusted | PCBs | | | |
| 3,3′,4,4′,5,5′,- | Dioxins, Furans, & Coplanar | 49.680 ± 36.591 | 114.363 ± 82.118 | 213.353 ± 128.960 |
| hexachlorobiphenyl | PCBs | | | |
| (hxcb) (fg/g) | | | | |
| PCB74 (ng/g) | Dioxins, Furans, & Coplanar | 0.028 ± 0.018 | 0.070 ± 0.053 | 0.158 ± 0.108 |
| | PCBs | | | |
| PCB170 Lipid | Dioxins, Furans, & Coplanar | 5.156 ± 3.576 | 12.887 ± 9.241 | 21.042 ± 12.775 |
| Adjusted | PCBs | | | |
| Triglycerides | Standard Biochemistry Profile | 119.421 ± 76.643 | 138.670 ± 87.891 | 153.678 ± 86.983 |
| (mg/dL) | | | | |
| PCB153 (ng/g) | Dioxins, Furans, & Coplanar | 0.108 ± 0.087 | 0.287 ± 0.221 | 0.518 ± 0.344 |
| | PCBs | | | |
| Oxychlordance (ng/g) | Dioxins, Furans, & Coplanar | 0.048 ± 0.034 | 0.115 ± 0.077 | 0.252 ± 0.165 |
| | PCBs | | | |
| PCB74 Lipid | Dioxins, Furans, & Coplanar | 4.894 ± 3.189 | 11.108 ± 8.304 | 22.965 ± 15.186 |
| Adjusted | PCBs | | | |
| Monocyte percent | Complete Blood Count with 5- | 7.040 ± 1.983 | 7.306 ± 2.009 | 7.897 ± 2.197 |
| (%) | part Differential - Whole Blood | | | |
| Ferritin (ng/mL) | Cadmium, Lead, Mercury, | 43.492 ± 39.542 | 66.328 ± 66.546 | 125.103 ± 113.683 |
| | Cotinine & Nutritional | | | |
| | Biochemistries | | | |
| 3,3′,4,4′,5,5′- | Dioxins, Furans, & Coplanar | 8.337 ± 6.123 | 17.921 ± 13.050 | 30.882 ± 18.476 |
| hexachlorobiphenyl | PCBs | | | |
| (hxcb) Lipid Adjusted | | | | |
| 2,3,4,7,8- | Dioxins, Furans, & Coplanar | 22.047 ± 14.452 | 38.539 ± 25.721 | 71.814 ± 41.045 |
| Pentachlorodibenzofuran | PCBs | | | |
| (pncdf) (fg/g) | | | | |
| Methylmalonic acid | Cadmium, Lead, Mercury, | 0.119 ± 0.055 | 0.131 ± 0.058 | 0.175 ± 0.095 |
| (umol/L) | Cotinine & Nutritional | | | |
| | Biochemistries | | | |
| PCB153 Lipid | Dioxins, Furans, & Coplanar | 18.171 ± 14.396 | 44.691 ± 33.060 | 75.553 ± 50.706 |
| Adjusted | PCBs | | | |
| PCB187 (ng/g) | Dioxines, Furans, & Coplanar | 0.024 ± 0.016 | 0.062 ± 0.051 | 0.120 ± 0.086 |
| | PCBs | | | |
| 2,3,4,7,8- | Dioxins, Furans, & Coplanar | 3.673 ± 2.366 | 6.068 ± 3.969 | 10.336 ± 5.730 |
| Pentachlorodibenzofuran | PCBs | | | |
| (pncdf) Lipid | | | | |
| Adjusted | | | | |
| PCB156 (ng/g) | Dioxins, Furans, & Coplanar | 0.020 ± 0.011 | 0.042 ± 0.030 | 0.076 ± 0.051 |
| | PCBs | | | |
| White blood cell | Complete Blood Count with 5- | 7.844 ± 2.361 | 7.221 ± 2.154 | 7.013 ± 1.997 |
| Count: SI | part Differential - Whole Blood | | | |
| PCB187 Lipid | Dioxins, Furans, & Coplanar | 4.237 ± 2.856 | 9.825 ± 7.893 | 17.376 ± 12.029 |
| Adjusted | PCBs | | | |
| 1,2,3,6,7,8- | Dioxins, Furans, & Coplanar | 102.674 ± 85.538 | 206.363 ± 161.318 | 413.352 ± 275.034 |
| Hexachlorodibenzo- | PCBs | | | |
| p-dioxin | | | | |
| (hxcdd)(fg/g) | | | | |
| Trans-nonachlor | Dioxins, Furans, & Coplanar | 13.054 ± 9.701 | 27.625 ± 19.276 | 56.993 ± 41.164 |
| Lipid Adjusted | PCBs | | | |
| PCB138 (ng/g) | Dioxins, Furans, & Coplanar | 0.080 ± 0.064 | 0.204 ± 0.157 | 0.380 ± 0.284 |
| | PCBs | | | |
| 4-pyridoxic acid | Vitamin B6 | 23.513 ± 18.708 | 29.795 ± 26.405 | 50.246 ± 48.362 |
| (nmol/L) | | | | |
| Potassium: SI | Standard Biochemistry Profile | 3.880 ± 0.283 | 3.920 ± 0.326 | 4.009 ± 0.386 |
| (mmol/L) | | | | |
| Trans-nonachlor | Dioxins, Furans, & Coplanar | 0.080 ± 0.065 | 0.179 ± 0.135 | 0.395 ± 0.308 |
| (ng/g) | PCBs | | | |
| 1,2,3,6,7,8- | Dioxins, Furans, & Coplanar | 17.061 ± 13.858 | 32.111 ± 24.257 | 60.093 ± 39.926 |
| Hexachlorodibenzo- | PCBs | | | |
| p-dioxin (hxcdd) | | | | |
| Lipid Adjusted | | | | |
| PCB138 Lipid | Dioxins, Furans, & Coplanar | 13.714 ± 10.729 | 32.122 ± 24.555 | 54.645 ± 39.907 |
| Adjusted | PCBs | | | |
| PCB118 (ng/g) | Dioxins, Furans, & Coplanar | 0.034 ± 0.028 | 0.090 ± 0.077 | 0.210 ± 0.172 |
| | PCBs | | | |
| PCB156 Lipid | Dioxins, Furans, & Coplanar | 3.484 ± 2.159 | 6.620 ± 4.574 | 11.148 ± 7.522 |
| Adjusted | PCBs | | | |
| PCB118 Lipid | Dioxins, Furans, & Coplanar | 5.932 ± 4.519 | 14.144 ± 12.460 | 30.600 ± 24.561 |
| Adjusted | PCBs | | | |
| Mean cell volume | Complete Blood Count with 5- | 88.106 ± 6.009 | 88.488 ± 6.549 | 90.100 ± 5.453 |
| (fL) | part Differential - Whole Blood | | | |
| PCB146 (ng/g) | Dioxins, Furans, & Coplanar | 0.019 ± 0.009 | 0.034 ± 0.027 | 0.063 ± 0.046 |
| | PCBs | | | |
| Blood cadmium | Blood Lead, Cadmium, Total | 0.404 ± 0.329 | 0.562 ± 0.449 | 0.539 ± 0.374 |
| (ug/L) | Mercury, Selenium, and | | | |
| | Manganese | | | |
| Two hour oral | Oral Glucose Tolerance Test | 102.604 ± 32.513 | 119.015 ± 43.371 | 142.831 ± 56.542 |
| glucose tolerance | | | | |
| (OGTT) (mg/dL) | | | | |
| Folate, serum | Cadmium, Lead, Mercury, | 13.491 ± 6.970 | 13.504 ± 7.135 | 18.243 ± 10.349 |
| (ng/mL) | Cotinine & Nutritional | | | |
| | Biochemistries | | | |
| PCB194 Lipid | Dioxins, Furans, & Coplanar | 3.137 ± 2.273 | 7.337 ± 5.183 | 13.407 ± 8.184 |
| Adjusted | PCBs | | | |
| PCB194 (ng/g) | Dioxins, Furans, & Coplanar | 0.023 ± 0.011 | 0.047 ± 0.033 | 0.092 ± 0.056 |
| | PCBs | | | |
| Hematocrit (%) | Complete Blood Count with 5- | 38.656 ± 3.365 | 39.492 ± 3.631 | 39.817 ± 3.579 |
| | part Differential - Whole Blood | | | |
| 1,2,3,4,7,8- | Dioxins, Furans, & Coplanar | 22.141 ± 11.933 | 33.539 ± 20.027 | 54.953 ± 34.654 |
| Hexachlorodibenzofuran | PCBs | | | |
| (hcxdf) (fg/g) | | | | |
| Perfluorohexane | Perfluoroalkyl and | 1.160 ± 1.061 | 1.411 ± 1.270 | 2.343 ± 1.90 |
| sulfonic acid (ug/L) | Polyfluoroalkyl Substances | | | |
| | (formerly Polyfluoroalkyl | | | |
| | Chemicals - PFC) | | | |
| RBC folate (nmol/L) | Folate - RBC | 1040.927 ± 427.954 | 1135.168 ± 494.490 | 1356.061 ± 634.740 |
| PCB99 (ng/g) | Dioxins, Furans, & Coplanar | 0.025 ± 0.014 | 0.050 ± 0.039 | 0.096 ± 0.079 |
| | PCBs | | | |
| p,p′-DDE (ng/g) | Dioxins, Furans, & Coplanar | 1.515 ± 1.712 | 5.086 ± 5.535 | 9.474 ± 9.065 |
| | PCBs | | | |
| p,p′-DDE Lipid | Dioxins, Furans, & Coplanar | 258.062 ± 303.889 | 805.676 ± 895.230 | 1378.711 ± 1273.385 |
| Adjusted | PCBs | | | |
| Total Serum Foalte | Folate - RBC | 39.463 ± 20.270 | 42.519 ± 23.431 | 55.419 ± 33.064 |
| (nmol/L) | | | | |
| PCB146 Lipid | Dioxins, Furans, & Coplanar | 3.336 ± 1.986 | 5.335 ± 3.768 | 9.005 ± 6.413 |
| Adjusted | PCBs | | | |
| PCB196 Lipid | Dioxins, Furans, & Coplanar | 3.135 ± 2.300 | 6.072 ± 4.240 | 11.181 ± 7.030 |
| Adjusted | PCBs | | | |
| PCB196 (ng/g) | Dioxins, Furans, & Coplanar | 0.022 ± 0.009 | 0.038 ± 0.027 | 0.076 ± 0.048 |
| | PCBs | | | |
| 1,2,3,4,6,7,8,9- | Dioxins, Furans, & Coplanar | 1642.940 ± 1237.084 | 2919.432 ± 2113.042 | 4700.494 ± 3387.159 |
| Octachlorodibenzo- | PCBs | | | |
| p-dioxin (ocdd) | | | | |
| (fg/g) | | | | |
| PCB183 (ng/g) | Dioxins, Furans, & Coplanar | 0.018 ± 0.008 | 0.024 ± 0.013 | 0.039 ± 0.025 |
| | PCBs | | | |
| Perfluorooctane | Polyfluoroalkyl Chemicals | 9.565 ± 7.478 | 12.748 ± 10.781 | 19.167 ± 14.873 |
| sulfonic acid | | | | |
| 3,3′,4,4′,5- | Dioxins, Furans, & Coplanar | 101.532 ± 79.531 | 200.988 ± 182.825 | 431.594 ± 378.770 |
| Pentachlorobiphenyl | PCBs | | | |
| (pncb) (fg/g) | | | | |
| trans- | Vitamin A, Vitamin E & | 24.038 ± 10.430 | 22.049 ± 10.431 | 18.394 ± 10.076 |
| lycopene(ug/dL) | Carotenoids | | | |
| 1,2,3,7,8- | Dioxins, Furans, & Coplanar | 16.983 ± 12.101 | 27.218 ± 20.779 | 56.075 ± 45.901 |
| Pentachlorodibenzo- | PCBs | | | |
| p-dioxin (pncdd) | | | | |
| (fg/g) | | | | |
| 1,2,3,4,6,7,8- | Dioxins, Furans, & Coplanar | 204.456 ± 149.166 | 335.511 ± 275.998 | 558.474 ± 408.415 |
| Heptachlororodibenzo- | PCBs | | | |
| p-dioxin (hpcdd) | | | | |
| (fg/g) | | | | |
| 3,3′,4,4′,5- | Dioxins, Furans, & Coplanar | 16.546 ± 12.217 | 32.001 ± 28.364 | 61.736 ± 53.368 |
| Pentachlorobiphenyl | PCBs | | | |
| (pncb) Lipid | | | | |
| Adjusted | | | | |
| 1,2,3,4,7,8- | Dioxins, Furans, & Coplanar | 3.733 ± 2.081 | 5.267 ± 3.025 | 8.011 ± 5.008 |
| Hexachlorodibenzofuran | PCBs | | | |
| (hcxdf) Lipid | | | | |
| Adjusted | | | | |
| 1,2,3,6,7,8- | Dioxins, Furans, & Coplanar | 18.988 ± 10.004 | 27.547 ± 17.069 | 43.103 ± 28.829 |
| Hexachlorodibenzofuran | PCBs | | | |
| (hxcdf) (fg/g) | | | | |
| PCB99 Lipid | Dioxins, Furans, & Coplanar | 4.493 ± 2.727 | 8.192 ± 6.524 | 14.129 ± 11.940 |
| Adjusted | PCBs | | | |
| Triiodothyronine | Thyroid Profile | 3.189 ± 0.357 | 3.071 ± 0.343 | 2.947 ± 0.323 |
| (T3), free (pg/mL) | | | | |
| 1,2,3,4,6,7,8,9- | Dioxins, Furans, & Coplanar | 271.323 ± 198.278 | 453.059 ± 308.513 | 679.182 ± 485.046 |
| Octachlorodibenzo- | PCBs | | | |
| p-dioxin (ocdd) Lipid | | | | |
| Adjusted | | | | |
| a-Tocopherol(ug/dL) | Vitamin A, Vitamin E & | 1145.100 ± 388.471 | 1364.457 ± 564.694 | 1771.421 ± 828.516 |
| | Carotenoids | | | |
| Blood o-Xylene | Blood, Water, & Related | 0.032 ± 0.021 | 0.034 ± 0.023 | 0.022 ± 0.014 |
| Result | Questionnaire Items | | | |
| Beta- | Dioxins, Furans, & Coplanar | 5.878 ± 6.043 | 16.008 ± 15.220 | 45.492 ± 39.271 |
| hexachlorocyclohexane | PCBs | | | |
| Lipid Adjusted | | | | |
| Plasma glucose: | Plasma Fasting Glucose, | 4.893 ± 0.539 | 5.402 ± 0.811 | 5.907 ± 1.311 |
| SI(mmol/L) | Serum C-peptide & Insulin | | | |
| 1,2,3,7,8- | Dioxins, Furans, & Coplanar | 2.920 ± 2.151 | 4.331 ± 3.323 | 7.942 ± 6.234 |
| Pentachlorodibenzo- | PCBs | | | |
| p-dioxin (pncdd) | | | | |
| Lipid Adjusted | | | | |
| Parathyroid | C-Reactive Protein (CRP), | 37.132 ± 18.562 | 46.062 ± 20.326 | 52.314 ± 26.434 |
| Hormone(Elecys | Bone Alkaline Phosphatase | | | |
| method) pg/mL | (BAP) & Parathyroid Hormone | | | |
| | (PTH) | | | |
| Beta-hexachloro- | Dioxins, Furans, & Coplanar | 0.037 ± 0.038 | 0.106 ± 0.107 | 0.311 ± 0.277 |
| cyclohexane (ng/g) | PCBs | | | |
| 1,2,3,4,6,7,8- | Dioxins, Furans, & Coplanar | 34.136 ± 23.957 | 52.261 ± 40.589 | 80.338 ± 56.975 |
| Heptachlororodibenzo- | PCBs | | | |
| p-dioxin (hpcdd) | | | | |
| Lipid Adjusted | | | | |
| PCB105 (ng/g) | Dioxins, Furans, & Coplanar | 0.018 ± 0.008 | 0.022 ± 0.013 | 0.041 ± 0.030 |
| | PCBs | | | |
| PCB177 (ng/g) | Dioxins, Furans, & Coplanar | 0.017 ± 0.009 | 0.021 ± 0.009 | 0.030 ± 0.016 |
| | PCBs | | | |
| Hemoglobin (g/dL) | Complete Blood Count with 5- | 13.112 ± 1.209 | 13.346 ± 1.332 | 13.452 ± 1.236 |
| | part Differential - Whole Blood | | | |
| Heptachlor Epoxide | Dioxins, Furans, & Coplanar | 0.029 ± 0.018 | 0.051 ± 0.045 | 0.088 ± 0.077 |
| (ng/g) | PCBs | | | |
| Perfluorooctanoic | Polyfluoroalkyl Chemicals | 2.686 ± 1.847 | 3.274 ± 2.216 | 4.254 ± 2.698 |
| acid | | | | |
| Heptachlor Epoxide | Dioxins, Furans, & Coplanar | 4.871 ± 2.948 | 8.151 ± 6.799 | 12.462 ± 10.228 |
| Lipid Adjusted | PCBs | | | |
| 1,2,3,6,7,8- | Dioxins, Furans, & Coplanar | 3.202 ± 1.711 | 4.356 ± 2.662 | 6.264 ± 4.179 |
| Hexachlorodibenzofuran | PCBs | | | |
| (hxcdf) Lipid | | | | |
| Adjusted | | | | |
| PCB183 Lipid | Dioxins, Furans, & Coplanar | 3.100 ± 1.816 | 4.029 ± 2.251 | 5.777 ± 3.730 |
| Adjusted | PCBs | | | |
| 2,3,7,8- | Dioxins, Furans, & Coplanar | 11.855 ± 6.651 | 15.055 ± 8.525 | 23.048 ± 15.298 |
| Tetrachlorodienzo- | PCBs | | | |
| p-dioxin (tcdd) (fg/g) | | | | |
| Vitamin B12, serum | Cadmium, Lead, Mercury, | 487.350 ± 232.796 | 554.723 ± 282.722 | 590.338 ± 317.893 |
| (pg/mL) | Cotinine & Nutritional | | | |
| | Biochemistries | | | |
| cis-b- | Vitamin A, Vitamin E & | 1.027 ± 0.739 | 1.243 ± 1.031 | 1.536 ± 1.287 |
| carotene(ug/dL) | Carotenoids | | | |
| Cotinine (ng/mL) | Cadmium, Lead, Mercury, | 0.071 ± 0.110 | 0.049 ± 0.066 | 0.044 ± 0.055 |
| | Cotinine & Nutritional | | | |
| | Biochemistries | | | |
| 1,2,3,7,8,9- | Dioxins, Furans, & Coplanar | 26.308 ± 14.683 | 34.028 ± 23.011 | 57.580 ± 45.524 |
| Hexachlorodibenzo- | PCBs | | | |
| p-dioxin (hxcdd) | | | | |
| (fg/g) | | | | |
| Triglyceride (mg/dL) | Cholesterol - LDL & | 115.093 ± 72.155 | 123.755 ± 74.037 | 143.842 ± 76.807 |
| | Triglycerides | | | |
| p,p′-DDT (ng/g) | Dioxins, Furans, & Coplanar | 0.037 ± 0.018 | 0.057 ± 0.046 | 0.103 ± 0.099 |
| | PCBs | | | |
| Triiodothyronine | Thyroid Profile | 119.130 ± 24.980 | 111.838 ± 23.364 | 105.728 ± 21.661 |
| (T3), total (ng/dL) | | | | |
| PCB105 Lipid | Dioxins, Furans, & Coplanar | 3.107 ± 1.735 | 3.625 ± 2.168 | 6.139 ± 4.794 |
| Adjusted | PCBs | | | |
| 1,2,3,4,7,8- | Dioxins, Furans, & Coplanar | 18.128 ± 7.504 | 27.221 ± 18.943 | 55.619 ± 45.629 |
| Hexachlorodibenzo- | PCBs | | | |
| p-dioxin | | | | |
| (hxcdd)(fg/g) | | | | |
| Mean cell | Complete Blood Count with 5- | 29.890 ± 2.484 | 29.909 ± 2.699 | 30.443 ± 2.208 |
| hemoglobin (pg) | part Differential - Whole Blood | | | |
| Dieldrin (ng/g) | Dioxins, Furans, & Coplanar | 0.032 ± 0.018 | 0.051 ± 0.034 | 0.079 ± 0.060 |
| | PCBs | | | |
| Folate, RBC (ng/mL | Cadmium, Lead, Mercury, | 296.475 ± 127.385 | 294.888 ± 117.588 | 347.105 ± 151.603 |
| RBC) | Cotinine & Nutritional | | | |
| | Biochemistries | | | |
| Aldrin | Dioxins, Furans, & Coplanar | 0.021 ± 0.000 | 0.021 ± 0.000 | 0.017 ± 0.005 |
| | PCBs | | | |
| trans-b- | Vitamin A, Vitamin E & | 16.046 ± 13.224 | 19.510 ± 17.853 | 22.915 ± 18.911 |
| carotene(ug/dL) | Carotenoids | | | |
| Eosinophils percent (%) | Complete Blood Count with 5- | 2.357 ± 1.697 | 2.532 ± 1.659 | 2.753 ± 1.727 |
| | part Differential - Whole Blood | | | |
| Endrin | Dioxins, Furans, & Coplanar | 0.021 ± 0.000 | 0.021 ± 0.000 | 0.017 ± 0.005 |
| | PCBs | | | |
| Bone alkaline | C-Reactive Protein (CRP) | 13.924 ± 5.802 | 15.427 ± 6.731 | 18.526 ± 7.676 |
| phosphotase (ug/L) | | | | |
| PCB199 Lipid | Dioxins, Furans, & Coplanar | 4.106 ± 1.741 | 7.521 ± 4.779 | 15.546 ± 10.372 |
| Adjusted | PCBs | | | |
| 1,2,3,4,7,8- | Dioxins, Furans, & Coplanar | 3.072 ± 1.313 | 4.311 ± 2.850 | 8.045 ± 6.492 |
| Hexachlorodibenzo- | PCBs | | | |
| p-dioxin (hxcdd) | | | | |
| Lipid Adjusted | | | | |
| 1,2,3,7,8,9- | Dioxins, Furans, & Coplanar | 4.447 ± 2.463 | 5.411 ± 3.499 | 8.282 ± 6.393 |
| Hexachlorodibenzo- | PCBs | | | |
| p-dioxin (hxcdd) | | | | |
| Lipid Adjusted | | | | |
| Dieldrin Lipid | Dioxins, Furans, & Coplanar | 5.271 ± 2.956 | 8.216 ± 5.671 | 11.434 ± 8.389 |
| Adjusted | PCBs | | | |
| p,p′-DDT Lipid | Dioxins, Furans, & Coplanar | 6.703 ± 4.150 | 9.310 ± 7.401 | 15.361 ± 14.598 |
| Adjusted | PCBs | | | |
| Segmented | Complete Blood Count with 5- | 59.968 ± 9.855 | 57.889 ± 9.257 | 57.827 ± 9.570 |
| neutrophils percent (%) | part Differential - Whole Blood | | | |
| 2,3,7,8- | Dioxins, Furans, & Coplanar | 2.019 ± 1.167 | 2.418 ± 1.408 | 3.385 ± 2.259 |
| Tetrachlorodienzo- | PCBs | | | |
| p-dioxin (tcdd) Lipid | | | | |
| Adjusted | | | | |
| Retinyl stearate | Cadmium, Lead, Mercury, | 0.393 ± 0.208 | 0.426 ± 0.258 | 0.495 ± 0.327 |
| (ug/dL) | Cotinine & Nutritional | | | |
| | Biochemistries | | | |
| PCB151 (ng/g) | Dioxins, Furans, & Coplanar | 0.021 ± 0.000 | 0.021 ± 0.000 | 0.014 ± 0.009 |
| | PCBs | | | |
| PCB149 (ng/g) | Dioxins, Furans, & Coplanar | 0.021 ± 0.000 | 0.021 ± 0.000 | 0.015 ± 0.008 |
| | PCBs | | | |
| Perfluorononanoic | Perfluoroalkyl and | 0.914 ± 0.613 | 1.159 ± 0.815 | 1.284 ± 0.919 |
| acid (ug/L) | Polyfluoroalkyl Substances | | | |
| | (formerly Polyfluoroalkyl | | | |
| | Chemicals - PFC) | | | |
| PCB177 Lipid | Dioxins, Furans, & Coplanar | 2.995 ± 1.791 | 3.494 ± 1.747 | 4.588 ± 2.662 |
| Adjusted | PCBs | | | |
| PCB178 Lipid | Dioxins, Furans, & Coplanar | 2.887 ± 1.771 | 3.364 ± 1.782 | 4.309 ± 2.366 |
| Adjusted | PCBs | | | |
| PCB209 (ng/g) | Non-dioxin-like | 0.004 ± 0.003 | 0.012 ± 0.009 | 0.068 ± 0.060 |
| | Polychlorinated Biphenyls | | | |
| PCB178 (ng/g) | Dioxins, Furans, & Coplanar | 0.021 ± 0.005 | 0.020 ± 0.009 | 0.029 ± 0.015 |
| | PCBs | | | |
| 5-Methyl | Serum Folate Forms | 36.437 ± 17.971 | 38.042 ± 20.225 | 49.779 ± 27.427 |
| THF(nmol/L) | | | | |
| PCB209 Lipid | Non-dioxin-like | 0.675 ± 0.482 | 1.943 ± 1.426 | 10.280 ± 9.471 |
| Adjusted (ng/g) | Polychlorinated Biphenyls | | | |
| C-peptide (nmol/L) | Plasma Fasting Glucose, | 0.733 ± 0.361 | 0.811 ± 0.400 | 0.946 ± 0.466 |
| in SI units | Serum C-peptide & Insulin | | | |
| Platelet count (%) SI | Complete Blood Count with 5- | 272.119 ± 69.134 | 274.817 ± 70.677 | 258.690 ± 67.026 |
| | part Differential - Whole Blood | | | |
| Blood | Blood, Water, & Related | 1.964 ± 2.532 | 1.623 ± 2.131 | 1.087 ± 1.502 |
| Bromodichloromethane | Questionnaire Items | | | |
| Result | | | | |
| Total iron binding | Cadmium, Lead, Mercury, | 391.897 ± 77.639 | 361.250 ± 59.773 | 358.743 ± 60.433 |
| capacity (ug/dL) | Cotinine & Nutritional | | | |
| | Biochemistries | | | |
| Red cell distribution | Complete Blood Count with 5- | 12.997 ± 1.286 | 13.261 ± 1.433 | 13.234 ± 1.155 |
| width (%) | part Differential - Whole Blood | | | |
| Blood Chloroform | Blood, Water, & Related | 12.301 ± 15.793 | 10.064 ± 13.356 | 6.841 ± 9.331 |
| Result | Questionnaire Items | | | |
| Glycidamide | Acrylamide & Glycidamide | 66.924 ± 38.948 | 64.408 ± 40.510 | 51.963 ± 30.198 |
| (pmoL/G Hb) | | | | |
| Testosterone total | Total Testosterone | 28.696 ± 15.883 | 19.191 ± 10.310 | 19.279 ± 14.053 |
| (ng/dL) | | | | |
| Hexachlorobenzene | Dioxins, Furans, & Coplanar | 0.103 ± 0.060 | 0.125 ± 0.072 | 0.158 ± 0.104 |
| (ng/g) | PCBs | | | |
| Apolipoprotein (B) | Cholesterol - LDL, Triglyceride | 86.453 ± 26.445 | 95.409 ± 25.071 | 95.865 ± 24.863 |
| (mg/dL) | & Apoliprotein (ApoB) | | | |
| ALT: SI (U/L) | Standard Biochemistry Profile | 19.105 ± 8.803 | 21.552 ± 9.416 | 20.709 ± 8.090 |
| 25-hydroxyvitamin | Vitamin D | 59.686 ± 27.497 | 62.622 ± 27.433 | 68.931 ± 29.472 |
| D2 + D3 | | | | |
| PCB206 Lipid | Dioxins, Furans, & Coplanar | 7.265 ± 5.696 | 7.936 ± 4.978 | 12.787 ± 8.581 |
| Adjusted | PCBs | | | |
| Follicle stimulating | Standard Biochemistry Profile | 5.603 ± 4.552 | 29.788 ± 29.725 | 54.277 ± 25.076 |
| hormone (mIU/mL) | | | | |
| Basophils percent | Complete Blood Count with 5- | 0.647 ± 0.385 | 0.709 ± 0.399 | 0.716 ± 0.390 |
| (%) | part Differential - Whole Blood | | | |
| 2-(N-Methyl- | Perfluoroalkyl and | 0.251 ± 0.228 | 0.290 ± 0.294 | 0.379 ± 0.390 |
| perfluorooctane | Polyfluoroalkyl Substances | | | |
| sulfonamido) acetic | (formerly Polyfluoroalkyl | | | |
| acid (ug/L) | Chemicals - PFC) | | | |
| Vitamin B6 | Vitamin B6 | 36.542 ± 38.552 | 50.581 ± 51.268 | 63.280 ± 60.104 |
| (Pyridoxal 5′- | | | | |
| phosphate) test | | | | |
| results (nmol/L). | | | | |
| Pyridoxal 5′- | Vitamin B6 | 48.256 ± 39.478 | 52.356 ± 47.389 | 64.061 ± 62.309 |
| phosphate (nmol/L) | | | | |
| total | Vitamin A, Vitamin E & | 43.120 ± 18.726 | 39.335 ± 18.316 | 33.710 ± 18.210 |
| Lycopene (ug/dL) | Carotenoids | | | |
| Blood Methyl t-Butyl | Blood, Water, & Related | 2.656 ± 3.936 | 1.849 ± 2.644 | 1.436 ± 2.216 |
| Ether (MTBE) | Questionnaire Items | | | |
| Results | | | | |
| Helicobacter pylori | C-Reactive Protein (CRP) | 0.865 ± 0.925 | 1.233 ± 1.095 | 1.410 ± 1.183 |
| (ISR) | | | | |
| PCB167 Lipid | Dioxins, Furans, & Coplanar | 2.826 ± 1.778 | 3.071 ± 1.709 | 3.833 ± 2.135 |
| Adjusted | PCBs | | | |
| Mirex (ng/g) | Dioxins, Furans, & Coplanar | 0.022 ± 0.010 | 0.026 ± 0.014 | 0.029 ± 0.015 |
| | PCBs | | | |
| Luteinizing hormone | Standard Biochemistry Profile | 6.927 ± 7.569 | 21.111 ± 19.077 | 32.187 ± 16.754 |
| (mlU/mL) | | | | |
| Blood manganese | Blood Lead, Cadmium, Total | 11.361 ± 4.367 | 10.713 ± 4.016 | 9.791 ± 3.668 |
| (ug/L) | Mercury, Selenium, and | | | |
| | Manganese | | | |
| Mean cell | Complete Blood Count with 5- | 34.005 ± 0.954 | 33.882 ± 0.993 | 33.859 ± 0.902 |
| hemoglobin | part Differential - Whole Blood | | | |
| concentration (g/dL) | | | | |
| PCB128 (ng/g) | Dioxins, Furans, & Coplanar | 0.020 ± 0.006 | 0.016 ± 0.010 | 0.016 ± 0.010 |
| | PCBs | | | |
| a- | Vitamin A, Vitamin E & | 2.965 ± 1.364 | 2.493 ± 1.199 | 2.394 ± 1.163 |
| Cryptoxanthin(ug/dL) | Carotenoids | | | |
| Thyroxine, free | Thyroid Profile | 0.780 ± 0.138 | 0.777 ± 0.150 | 0.833 ± 0.177 |
| (ng/dL) | | | | |
| cis-Lycopene(ug/dL) | Vitamin A, Vitamin E & | 20.188 ± 9.150 | 18.692 ± 9.036 | 16.255 ± 8.935 |
| | Carotenoids | | | |
| Thyroid stimulating | Thyroid Profile | 1.644 ± 1.125 | 1.827 ± 1.172 | 2.062 ± 1.429 |
| hormone (uIU/mL) | | | | |
| PCB172 Lipid | Dioxins, Furans, & Coplanar | 2.830 ± 1.815 | 3.082 ± 1.672 | 3.645 ± 1.961 |
| Adjusted | PCBs | | | |
| Blood mercury, total | Blood Lead, Cadmium, Total | 1.120 ± 1.082 | 1.447 ± 1.378 | 1.313 ± 1.250 |
| (ug/L) | Mercury, Selenium, and | | | |
| | Manganese | | | |
| Inorganic mercury, | Blood mercury: inorganic, | 0.285 ± 0.082 | 0.303 ± 0.113 | 0.303 ± 0.123 |
| blood (ug/L) | ethyl and methyl | | | |
| 2,2′,4,4′,5,5′- | Brominated Flame Retardants | 10.386 ± 11.824 | 20.076 ± 20.545 | 23.387 ± 19.930 |
| hexabromobiphenyl | (BFRs) | | | |
| (pg/g) | | | | |
| Vitamin C (mg/dL) | Vitamin C | 0.976 ± 0.428 | 0.955 ± 0.488 | 1.125 ± 0.575 |
| Blood m-/p-Xylene | Blood, Water, & Related | 0.113 ± 0.096 | 0.125 ± 0.109 | 0.092 ± 0.082 |
| Result | Questionnaire Items | | | |
| PCB167 (ng/g) | Dioxins, Furans, & Coplanar | 0.020 ± 0.006 | 0.018 ± 0.008 | 0.024 ± 0.011 |
| | PCBs | | | |
| Mercury, methyl | Blood mercury: inorganic, | 0.841 ± 0.961 | 1.140 ± 1.285 | 1.238 ± 1.377 |
| (ug/L) | ethyl and methyl | | | |
| Combined | Vitamin A, Vitamin E & | 15.999 ± 8.037 | 16.252 ± 8.502 | 17.932 ± 9.393 |
| Lutein/zeaxanthin(ug/dL) | Carotenoids | | | |
| 2,2′,4,4′,5,6′- | Brominated Flame Retardants | 3.918 ± 3.141 | 3.283 ± 2.220 | 2.177 ± 1.278 |
| hexabromodiphenyl | (BFRs) | | | |
| ether (pg/g) | | | | |
| Folic acid, serum | Folate - RBC | 1.016 ± 0.564 | 1.052 ± 0.626 | 1.255 ± 0.796 |
| (nmol/L) | | | | |
| Acrylamide | Acrylamide & Glycidamide | 63.778 ± 38.131 | 69.892 ± 46.776 | 54.627 ± 30.996 |
| (pmoL/G Hb) | | | | |
| 2,2′,4,4′,5,5′- | Brominated Flame Retardants | 1.785 ± 2.111 | 3.271 ± 3.311 | 3.488 ± 2.911 |
| hexabromobiphenyl | (BFRs) | | | |
| lipid adjusted (ng/g) | | | | |
| 2,3,4,6,7,8,- | Dioxins, Furans, & Coplanar | 13.535 ± 6.010 | 14.578 ± 6.595 | 16.184 ± 7.933 |
| Hexchlorodibenzofuran | PCBs | | | |
| (hxcdf) (fg/g) | | | | |
| total b- | Vitamin A, Vitamin E & | 16.938 ± 14.129 | 18.016 ± 16.188 | 22.552 ± 18.021 |
| Carotene(ug/dL) | Carotenoids | | | |
| 25-hydroxyvitamin | Vitamin D | 57.117 ± 27.279 | 58.804 ± 26.977 | 62.682 ± 29.259 |
| D3(nmol/L) | | | | |
| Perfluoroundecanoic | Perfluoroalkyl and | 0.181 ± 0.131 | 0.220 ± 0.197 | 0.219 ± 0.181 |
| acid (ug/L) | Polyfluoroalkyl Substances | | | |
| | (formerly Polyfluoroalkyl | | | |
| | Chemicals - PFC) | | | |
| Protoporphyrin | Cadmium, Lead, Mercury, | 63.071 ± 28.477 | 59.968 ± 26.152 | 58.177 ± 21.503 |
| (ug/dL RBC) | Cotinine & Nutritional | | | |
| | Biochemistries | | | |
| PCB206 (ng/g) | Dioxins, Furans, & Coplanar | 0.070 ± 0.002 | 0.068 ± 0.009 | 0.089 ± 0.056 |
| | PCBs | | | |
| PCB157 Lipid | Dioxins, Furans, & Coplanar | 2.818 ± 1.770 | 3.017 ± 1.636 | 3.410 ± 1.706 |
| Adjusted | PCBs | | | |
| Phytofluene(ug/dL) | Vitamin A, Vitamin E & | 5.556 ± 3.359 | 4.674 ± 2.782 | 4.549 ± 2.801 |
| | Carotenoids | | | |
| Aldrin Lipid | Dioxins, Furans, & Coplanar | 2.913 ± 1.085 | 2.716 ± 0.984 | 2.519 ± 0.904 |
| Adjusted | PCBs | | | |
| epi-25- | Vitamin D | 3.115 ± 2.308 | 3.170 ± 2.287 | 3.563 ± 2.527 |
| hydroxyvitamin D3 | | | | |
| (nmol/L) | | | | |
| PCB172 (ng/g) | Dioxins, Furans, & Coplanar | 0.020 ± 0.006 | 0.019 ± 0.009 | 0.023 ± 0.011 |
| | PCBs | | | |
| PCB66 (ng/g) | Dioxins, Furans, & Coplanar | 0.023 ± 0.013 | 0.024 ± 0.014 | 0.027 ± 0.015 |
| | PCBs | | | |
| Endrin Lipid | Dioxins, Furans, & Coplanar | 2.907 ± 1.073 | 2.701 ± 0.981 | 2.521 ± 0.906 |
| Adjusted | PCBs | | | |
| a-carotene(ug/dL) | Vitamin A, Vitamin E & | 3.954 ± 3.693 | 4.471 ± 4.184 | 4.659 ± 4.147 |
| | Carotenoids | | | |
| Trans 9, trans 12- | Trans Fatty Acids | 2.142 ± 1.304 | 2.260 ± 1.250 | 2.590 ± 1.425 |
| octadienoic acid (uM) | | | | |
| PCB28 (ng/g) | Dioxins, Furans, & Coplanar | 0.045 ± 0.020 | 0.048 ± 0.022 | 0.054 ± 0.021 |
| | PCBs | | | |
| Pefluorodecanoic | Perfluoroalkyl and | 0.275 ± 0.210 | 0.314 ± 0.241 | 0.324 ± 0.244 |
| acid (ug/L) | Polyfluoroalkyl Substances | | | |
| | (formerly Polyfluoroalkyl | | | |
| | Chemicals - PFC) | | | |
| Lymphocyte percent | Complete Blood Count with 5- | 29.932 ± 8.634 | 31.539 ± 8.307 | 30.773 ± 8.718 |
| (%) | part Differential - Whole Blood | | | |
| Thyroid stimulating | Thyroid - Stimulating Hormone | 1.570 ± 0.981 | 1.653 ± 0.994 | 2.040 ± 1.495 |
| hormone (IU/L) | & Thyroxine (TSH & T4) | | | |
| 1,2,3,4,6,7,8- | Dioxins, Furans, & Coplanar | 48.186 ± 33.484 | 55.356 ± 37.389 | 60.089 ± 41.132 |
| Heptachlorodibenzofuran | Dioxins, Furans, & Coplanar | 48.186 ± 33.484 | 55.356 ± 37.389 | 60.089 ± 41.132 |
| (hpcdf) (fg/g) | PCBs | | | |
| Hexachlorobenzene | Dioxins, Furans, & Coplanar | 19.352 ± 13.087 | 19.999 ± 11.913 | 23.534 ± 14.948 |
| Lipid Adjusted | PCBs | | | |
| Mirex Lipid Adjusted | Dioxins, Furans, & Coplanar | 3.849 ± 2.025 | 4.268 ± 2.627 | 4.615 ± 2.891 |
| | PCBs | | | |
| Total dust weight (mg) | Allergens - Household Dust | 976.039 ± 611.659 | 936.203 ± 598.270 | 794.823 ± 501.310 |
| Insulin: SI(pmol/L) | Plasma Fasting Glucose, | 70.044 ± 43.839 | 73.187 ± 48.045 | 84.502 ± 57.466 |
| | Serum C-peptide & Insulin | | | |
| Sieved dust weight (mg) | Allergens - Household Dust | 662.129 ± 473.811 | 610.217 ± 408.471 | 534.390 ± 345.341 |
| Serum Selenium (ug/L) | Copper, Selenium & Zinc - | 123.713 ± 16.087 | 128.921 ± 19.590 | 129.085 ± 17.004 |
| | Serum | | | |
| Lutein(ug/dL) | Vitamin A, Vitamin E & | 10.136 ± 4.818 | 10.150 ± 5.375 | 11.575 ± 6.343 |
| | Carotenoids | | | |
| Blood Nitromethane | Volatile Organic Compounds - | 722.413 ± 257.983 | 784.071 ± 336.382 | 853.652 ± 432.620 |
| (pg/mL) | Trihalomethanes/MTBE/Nitro | | | |
| | methane - Blood | | | |
| Gamma- | Dioxins, Furans, & Coplanar | 3.457 ± 1.586 | 3.238 ± 1.422 | 3.086 ± 1.400 |
| hexachlorocyclohexane | PCBs | | | |
| Lipid Adjusted | | | | |
| Retinyl palmitate | Cadmium, Lead, Mercury, | 1.879 ± 1.533 | 1.858 ± 1.519 | 2.088 ± 1.781 |
| (ug/dL) | Cotinine & Nutritional | | | |
| | Biochemistries | | | |
| Trans 9- | Trans Fatty Acids | 23.355 ± 17.669 | 23.707 ± 16.674 | 27.774 ± 17.528 |
| octadecenoic acid (uM) | | | | |
| 1,2,3,7,8,9- | Dioxins, Furans, & Coplanar | 13.244 ± 5.512 | 13.909 ± 5.915 | 14.803 ± 6.702 |
| Hexachlorodibenzofuran | PCBs | | | |
| (hxcdf) (fg/g) | | | | |
| 1,2,3,4,7,8,9- | Dioxins, Furans, & Coplanar | 12.476 ± 4.383 | 13.546 ± 5.010 | 13.992 ± 5.196 |
| Heptachlorodibenzofuran | PCBs | | | |
| (Hpcdf) (fg/g) | | | | |
| PCB87 (ng/g) | Dioxins, Furans, & Coplanar | 0.021 ± 0.000 | 0.021 ± 0.000 | 0.020 ± 0.003 |
| | PCBs | | | |
| Red cell count SI | Complete Blood Count with 5- | 4.399 ± 0.399 | 4.474 ± 0.401 | 4.431 ± 0.433 |
| | part Differential - Whole Blood | | | |

In some embodiments, selected biomarkers related to aging and mortality risks can be used to evaluate if blood, serum or plasma is “aged” or “old” or the composition comprising it comprises one or two or more biomarkers characteristic of aged subject. Table 2 (below) shows some of the selected biomarkers. In some embodiments, blood is said to be “aged” or “old” or the composition is said to comprise one or two or more biomarkers characteristic of aged subject when blood fraction from which it is made or composition comprising plasma fraction has a concentration of its elements that falls within the range of concentrations related to the moderate or high risk of mortality described in the available sources regarding the correlation of corresponding parameters with mortality, e.g. as described in https://www.ncbi.nlm.nih.gov/pmc/articles/PMC5611985/ or on the website http://mortalitypredictors.org and the publications cited there on blood predictors of mortality or in any other source.

In some embodiments, the compounds and compositions of this disclosure are useful for changing selected biomarkers related to aging or mortality or morbidity risks, including but not limited to described in this disclosure into a younger state and thus reducing the risks of mortality and/or morbidity.
In some embodiments, biomarkers mentioned in this description, could be used to identify the biological age of a subject and/or to verify whether a treated subject responds to treatment (e.g. if one or more of the biomarkers change to alevel characteristic of a younger age or delay in changing into the level characteristic of older age).
In some embodiments, biomarkers of aging, biological age metrics, chronological age metrics, mortality biomarkers, morbidity biomarkers, health declines, biomarkers of stress resistance, biomarkers of resilience, frailty index, frailty biomarkers, biomarkers of particular age related diseases and conditions can be used to verify whether a treated subject responds to treatment (e.g. if one or more of the biomarkers change to a level characteristic of a younger age or delay in changing into the level characteristic of older age).

Every web link cited in this application, in case of inaccessibility as a rule can be retrieved via https://web.archive.org or similar internet archive services.

In some embodiments, the one or two or more biomarkers, as referred to in reference to biomarkers characteristic of an aged subject, (with associated measurement units in plasma) are selected from the group: Glucose, serum (mg/dL); Creatinine (mg/dL); Lactate dehydrogenase LDH (U/L); Uric acid (mg/dL); Blood lead (ug/dL); Homocysteine (umol/L); Vitamin A (ug/dL); Fasting Glucose (mg/dL); GGT: SI (U/L); Total cholesterol (mg/dL); Vitamin E (ug/dL); Chloride: SI (mmol/L); AST: SI (U/L); Sodium: SI (mmol/L); PCB180 (ng/g); Cholesterol (mg/dL); PCB170 (ng/g); Alkaline phosphatase (U/L); PCB180 Lipid Adjusted; Oxychlordane Lipid Adjusted; 3,3′, 4,4′, 5,5′-hexachlorobiphenyl (hxcb) (fg/g); PCB74 (ng/g); PCB170 Lipid Adjusted; Triglycerides (mg/dL); PCB153 (ng/g); Oxychlordane (ng/g); PCB74 Lipid Adjusted; Monocyte percent (%); Ferritin (ng/mL); 3,3′, 4,4′, 5,5′-hexachlorobiphenyl (hxcb) Lipid Adjusted; 2,3,4,7,8-Pentachlorodibenzofuran (pncdf) (fg/g); Methylmalonic acid (umol/L); PCB153 Lipid Adjusted; PCB187 (ng/g); 2,3,4,7,8-Pentachlorodibenzofuran (pncdf) Lipid Adjusted; PCB156 (ng/g); White blood cell count: SI; PCB187 Lipid Adjusted; 1,2,3,6,7,8-Hexachlorodibenzo-p-dioxin (hxcdd)(fg/g); Trans-nonachlor Lipid Adjusted; PCB138 (ng/g); 4-pyridoxic acid (nmol/L); Potassium: SI (mmol/L); Trans-nonachlor (ng/g); 1,2,3,6,7,8-Hexachlorodibenzo-p-dioxin (hxcdd) Lipid Adjusted; PCB138 Lipid Adjusted; PCB118 (ng/g); PCB156 Lipid Adjusted; PCB118 Lipid Adjusted; Mean cell volume (fL); PCB146 (ng/g); Blood cadmium (ug/L); Two hour oral glucose tolerance (OGTT) (mg/dL); Folate, serum (ng/mL); PCB194 Lipid Adjusted; PCB194 (ng/g); Hematocrit (%); 1,2,3,4,7,8-Hexachlorodibenzofuran (hcxdf) (fg/g); Perfluorohexane sulfonic acid (ug/L); RBC folate (nmol/L); PCB99 (ng/g); p,p′-DDE (ng/g); p,p′-DDE Lipid Adjusted; Total Serum Foalte (nmol/L); PCB146 Lipid Adjusted; PCB196 Lipid Adjusted; PCB196 (ng/g); 1,2,3,4,6,7,8,9-Octachlorodibenzo-p-dioxin (ocdd) (fg/g); PCB183 (ng/g); Perfluorooctane sulfonic acid; 3,3′, 4,4′, 5-Pentachlorobiphenyl (pncb) (fg/g); trans-lycopene (ug/dL); 1,2,3,7,8-Pentachlorodibenzo-p-dioxin (pncdd) (fg/g); 1,2,3,4,6,7,8-Heptachlororodibenzo-p-dioxin (hpcdd) (fg/g); 3,3′, 4,4′, 5-Pentachlorobiphenyl (pncb) Lipid Adjusted; 1,2,3,4,7,8-Hexachlorodibenzofuran (hcxdf) Lipid Adjusted; 1,2,3,6,7,8-Hexachlorodibenzofuran (hxcdf) (fg/g); PCB99 Lipid Adjusted; Triiodothyronine (T3), free (pg/mL); 1,2,3,4,6,7,8,9-Octachlorodibenzo-p-dioxin (ocdd) Lipid Adjusted; a-Tocopherol (ug/dL); Blood o-Xylene Result; Beta-hexachlorocyclohexane Lipid Adjusted; Plasma glucose: SI (mmol/L); 1,2,3,7,8-Pentachlorodibenzo-p-dioxin (pncdd) Lipid Adjusted; Parathyroid Hormone (Elecys method) pg/mL; Beta-hexachloro-cyclohexane (ng/g); 1,2,3,4,6,7,8-Heptachlororodibenzo-p-dioxin (hpcdd) Lipid Adjusted; PCB105 (ng/g); PCB177 (ng/g); Hemoglobin (g/dL); Heptachlor Epoxide (ng/g); Perfluorooctanoic acid; Heptachlor Epoxide Lipid Adjusted; 1,2,3,6,7,8-Hexachlorodibenzofuran (hxcdf) Lipid Adjusted; PCB183 Lipid Adjusted; 2,3,7,8-Tetrachlorodienzo-p-dioxin (tcdd) (fg/g); Vitamin B12, serum (pg/mL); cis-b-carotene (ug/dL); Cotinine (ng/mL); 1,2,3,7,8,9-Hexachlorodibenzo-p-dioxin (hxcdd) (fg/g); Triglyceride (mg/dL); p,p′-DDT (ng/g); Triiodothyronine (T3), total (ng/dL); PCB105 Lipid Adjusted; 1,2,3,4,7,8-Hexachlorodibenzo-p-dioxin (hxcdd)(fg/g); Mean cell hemoglobin (pg); Dieldrin (ng/g); Folate, RBC (ng/mL RBC); Aldrin; trans-b-carotene (ug/dL); Eosinophils percent (%); Endrin; Bone alkaline phosphotase (ug/L); PCB199 Lipid Adjusted; 1,2,3,4,7,8-Hexachlorodibenzo-p-dioxin (hxcdd) Lipid Adjusted; 1,2,3,7,8,9-Hexachlorodibenzo-p-dioxin (hxcdd) Lipid Adjusted; Dieldrin Lipid Adjusted; p,p′-DDT Lipid Adjusted; Segmented neutrophils percent (%); 2,3,7,8-Tetrachlorodienzo-p-dioxin (tcdd) Lipid Adjusted; Retinyl stearate (ug/dL); PCB151 (ng/g); PCB149 (ng/g); Perfluorononanoic acid (ug/L); PCB177 Lipid Adjusted; PCB178 Lipid Adjusted; PCB209 (ng/g); PCB178 (ng/g); 5-Methyl THF(nmol/L); PCB209 Lipid Adjusted (ng/g); C-peptide (nmol/L) in SI units; Platelet count (%) SI; Blood Bromodichloromethane Result; Total iron binding capacity (ug/dL); Red cell distribution width (%); Blood Chloroform Result; Glycidamide (pmoL/G Hb); Testosterone total (ng/dL); Hexachlorobenzene (ng/g); Apolipoprotein (B) (mg/dL); ALT: SI (U/L); 25-hydroxyvitamin D2+D3; PCB206 Lipid Adjusted; Follicle stimulating hormone (mIU/mL); Basophils percent (%); 2-(N-Methyl-perfluorooctane sulfonamido) acetic acid (ug/L); Vitamin B6 (Pyridoxal 5′-phosphate) test results (nmol/L); Pyridoxal 5′-phosphate (nmol/L); total Lycopene (ug/dL); Blood Methyl t-Butyl Ether (MTBE) Result; Helicobacter pylori (ISR); PCB167 Lipid Adjusted; Mirex (ng/g); Luteinizing hormone (mIU/mL); Blood manganese (ug/L); Mean cell hemoglobin concentration (g/dL); PCB128 (ng/g); a-Cryptoxanthin (ug/dL); Thyroxine, free (ng/dL); cis-Lycopene (ug/dL); Thyroid stimulating hormone (ulU/mL); PCB172 Lipid Adjusted; Blood mercury, total (ug/L); Inorganic mercury, blood (ug/L); 2,2′, 4,4′, 5,5′-hexabromobiphenyl (pg/g); Vitamin C (mg/dL); Blood m-/p-Xylene Result; PCB167 (ng/g); Mercury, methyl (ug/L); Combined Lutein/zeaxanthin (ug/dL); 2,2′, 4,4′, 5,6′-hexabromodiphenyl ether (pg/g); Folic acid, serum (nmol/L); Acrylamide (pmoL/G Hb); 2,2′, 4,4′, 5,5′-hexabromobiphenyl lipid adjusted (ng/g); 2,3,4,6,7,8-Hexchlorodibenzofuran (hxcdf) (fg/g); total b-Carotene (ug/dL); 25-hydroxyvitamin D3(nmol/L); Perfluoroundecanoic acid (ug/L); Protoporphyrin (ug/dL RBC); PCB206 (ng/g); PCB157 Lipid Adjusted; Phytofluene (ug/dL); Aldrin Lipid Adjusted; epi-25-hydroxyvitamin D3 (nmol/L); PCB172 (ng/g); PCB66 (ng/g); Endrin Lipid Adjusted; a-carotene (ug/dL); Trans 9, trans 12-octadienoic acid (uM); PCB28 (ng/g); Pefluorodecanoic acid (ug/L); Lymphocyte percent (%); Thyroid stimulating hormone (IU/L); 1,2,3,4,6,7,8-Heptachlorodibenzofuran (hpcdf) (fg/g); Hexachlorobenzene Lipid Adjusted; Mirex Lipid Adjusted; Total dust weight (mg); Insulin: SI (pmol/L); Sieved dust weight (mg); Serum Selenium (ug/L); Lutein(ug/dL); Blood Nitromethane (pg/mL); Gamma-hexachlorocyclohexane Lipid Adjusted; Retinyl palmitate (ug/dL); Trans 9-octadecenoic acid (uM); 1,2,3,7,8,9-Hexachlorodibenzofuran (hxcdf) (fg/g); 1,2,3,4,7,8,9-Heptachlorodibenzofuran (Hpcdf) (fg/g); PCB87 (ng/g); and Red cell count SI. In some embodiments, the two or more biomarkers are selected from the group: Glucose, serum (mg/dl); Creatinine (mg/dl); Lactate dehydrogenase LDH (U/L); Uric acid (mg/dl); Blood lead (ug/dl); Homocysteine(umol/L); Vitamin A (ug/dl); Fasting Glucose (mg/dl); GGT: SI (U/L); Total cholesterol (mg/dl); Vitamin E (ug/dl); Chloride: SI (mmol/L); AST: SI (U/L); Sodium: SI (mmol/L); PCB180 (ng/g); Cholesterol (mg/dl); PCB170 (ng/g); Alkaline phosphatase(U/L) and glycohemoglobin. In some embodiments, biomarkers characteristic of aging are selected from: glucose serum, glycohemoglobin, creatine, lactate dehydrogenase, uric acid, blood lead, homocysteine, vitamin A, fasting glucose, gamma glutamyltransferase (GGT), total cholesterol, Vitamin E, chloride, aspartate aminotransferase (AST), sodium, and 2,2′, 3,4,4′, 5,5′-heptachlorobiphenyl (PCB180). In some embodiments, biomarkers characteristic of aging are selected from: glucose serum, glycohemoglobin, creatine, lactate dehydrogenase, uric acid, blood lead, homocysteine, vitamin A, fasting glucose, gamma glutamyltransferase (GGT), and total cholesterol. In some embodiments, biomarkers characteristic of aging are selected from: glucose serum, glycohemoglobin, creatine, lactate dehydrogenase, uric acid, melatonin and blood lead.

TABLE 2
Non-limiting list of selected biomarkers related to mortality risks
Name
Red blood cell distribution width
Mean reticulocytes volume
Neutrophil number
Alpha-1-acid glycoprotein
White blood cell count
Hemoglobin
Red blood cell count
Monocyte count
Basophil count
Neutrophils percentage
Albumin, serum/plasma
Lymphocyte percentage
Hematocrit
Leukocyte telomere length
Mean sphered cells volume
Very-low-density lipoprotein
Insulin-like growth factor 1
Mean corpuscular hemoglobin
Mean corpuscular volume
Citrate
trans-lycopene
Monocyte percentage
Platelet count
Reticulocytes count
Lymphocyte count
Platelet distribution width
Plateletcrit
Immature reticulocytes fraction
Reticulocytes, high light scatter, percentage
Reticulocytes, high light scatter, number
Reticulocytes percentage
Soluble CD14
Eosinophils percentage
25-hydroxyvitamin D
Adiponectin
Ascorbic acid
Brain natriuretic peptide
C-reactive protein
Cardiac troponin I
Estimated glomerular filtration rate
Fibroblast growth factor-23
Gamma-glutamyltransferase
Glucose
Growth differentiation factor 15
H-FABP
Homeostatic model assessment of insulin resistance
Homocysteine
Lipoprotein-associated phospholipase A2 activity
N-terminal atrial natriuretic peptide
SUA
Type I collagen degradation
Vitamin A
High-density lipoprotein cholesterol
Klotho
Leptin
Club (a.k.a. Clara) cell secretory protein
Antinuclear autoantibodies
Soluble ST2
Alanine transaminase
Alkaline phosphatase
Alpha-1-antichymotrypsin
Angiopoietin-2
ApoB/ApoA1 ratio
Asymmetric dimethylarginine
C-reactive protein, high-sensitivity
Cardiac troponin T, high-sensitivity
Cholesterol
Creatinine
Cystatin C
Fibrinogen
Glycated hemoglobin
Growth hormone
Homoarginine
Insulin-like growth factor binding protein 1
Interleukin-6
Low-density lipoprotein cholesterol
Lycopene
Mitochondrial DNA copy number
N-terminal pro-brain natriuretic peptide
Neutrophil gelatinase-associated lipocalinin
Osteocalcin
Osteoprotegerin
Phosphorus
Testosterone
Triglycerides
Tumor necrosis factor alpha
Uric acid
alpha-carotene
beta-trace protein
β2-microglobulin
Anion gap, serum albumin adjusted
CD4:CD8 ratio
CD8 cells
Carboxyl-terminal telopeptide of collagen type I
Plasma viscosity
Insulin-like growth factor binding protein 2
Peroxiredoxin 4
Stromal cell derived factor
Carotenoids
Oxygenated carotenoids
Urea
sj/β-TREC ratio
Insulin-like growth factor binding protein 3
Proinsulin
Factor VIIc
IgA to tissue transglutaminase
Bilirubin
Mean platelet volume
Galectin-3
Interleukin-8
Loss of Y chromosome
Soluble tumour necrosis factor receptor 1
Symmetrical dimethyl arginine
T cells
Thyroxine
Cell-free DNA concentration
beta-cryptoxanthin
Basophil percentage
Interleukin-10
Apolipoprotein A-1
Amino-terminal propeptide of type I collagen
Estradiol-to-sex hormone binding globulin ratio
Neutrophilia
Butyrylcholinesterase activity
Reticulocytes number
Parathyroid hormone
Follicle stimulating hormone
Interleukin 1-beta
17beta-E(2)
Amino-terminal peptide of procollagen type III

In some embodiments this invention is a kit, comprising an agent, disclosed or described in this disclosure, including but not limited to binding element, or composition disclosed in this application or its functional or structural analog or prodrug and the notice, description or instruction regarding the reduction or modulating or binding or inhibiting or degrading in blood plasma of at least one protein, selected of the group consisting of Targets, by the means of such agent or composition, optionally for anti-aging treatment, optionally comprising at least the medication labeling information, optionally wherein such notice, description or instruction comprises information about administration of corresponding agent or composition in a dosage and regimen to produce the biological effect comparable or alike or close to the reduction of protein selected from Targets to negligible amount in bloodstream of the subject.

In some embodiments this invention is a kit, comprising an agent, modulating or binding or inhibiting or degrading or activating at least one effector upstream or downstream of at least one of the Targets, optionally, wherein such modulation or binding or inhibiting or degrading or activating has anti-aging effect and the notice, description or instruction regarding the modulation or binding or inhibiting or degrading or activating or reduction in blood plasma of at least one such protein or other molecule, by the means of such agent or composition, optionally for anti-aging treatment, optionally comprising at least the medication labeling information, optionally wherein such notice, description or instruction comprises information about administration of corresponding agent or composition in dosage and regimen to produce the biological effect comparable or alike or close to the reduction of protein selected from Targets to negligible amount in bloodstream of the subject.

In some embodiments, this invention is a kit, comprising the device of this disclosure, including but not limited to protein binding device or filtering device, and a notice, description or instruction regarding the reduction in blood plasma of at least one protein, selected of the group consisting of Targets, by the means of such device or by the means of such device in connection with apheresis or alike system. In some embodiments a notice, description or instruction regarding the reduction in blood plasma of at least one protein, selected of the group consisting of Targets contains the information regarding reduction of Target or regarding reduction of Target to negligible amount or less. In some embodiments such notice, description or instruction comprises information related to the reduction in blood plasma of at least one Target or any for anti-aging treatment.

In some embodiments, this invention is a kit, comprising an agent of this invention and a notice, description or instruction for its use by human or by other animal subject in a dosage and regimen to maintain concentration of such agent in blood of such subject. In some embodiments such notice, description or instruction comprises information related to the deactivation, inhibition or degradation of at least one Target in blood plasma, optionally for anti-aging treatment.

In some embodiments, this invention is a kit, comprising the device of this disclosure, including but not limited to protein binding or filtering device of this disclosure and a notice, description or instruction regarding the reduction in blood plasma of at least one at least one effector upstream or downstream of at least one of the Targets, by the means of such device or by the means of such device in connection with apheresis or alike system. In some embodiments a notice, description or instruction regarding the reduction in blood plasma of at least one such protein or other molecule contains the information regarding reduction of such protein or other molecule or regarding reduction of such protein or molecule to negligible amount or less.

The kit of this invention can be made of any material such as paper, plastic, steel, glass, etc, including but not limited to materials used in the art for kits for medical devices, including but not limited to Known Apheresis devices (as it is defined further in this disclosure).

In some embodiments, this invention is a protein binding or other device of this disclosure with the mentioned notice, description or instruction attached to such device or imprinted or drawn or in any other way displayed on such device or in any other way associated with such device (e.g. in machine readable form). One of the primary purposes of some aspects of his invention is to provide medical device for treatment including but not limited to anti-aging treatment. As a rule the medical device or kit comprising medical device of this invention should be accompanied with the notice, description or instruction (e.g., treatment and/or operation guidelines).

In some embodiments the contents and appearance of such notice, description or instruction is regulated by the respective national or international rules regarding labeling of medical devices, incorporated here by reference or such notice, description or instruction comprise at least part or optionally most of the or optionally all the information required by applicable medical device or medicines labeling regulations. For example, The Federal Food, Drug and Cosmetic Act (FFDCA) in USA is the law under which the FDA takes action against regulated products. Specifically: Section 201(k) defines ‘label’ as a: ‘display of written, printed, or graphic matter upon the immediate container of any article . . . ’ The term ‘immediate container’ does not include package liners. Any word, statement, or other information appearing on the immediate container must also appear ‘on the outside container or wrapper, if any there be, or the retail package of such article, or is easily legible through the outside container of wrapper.’ Section 201(m) defines ‘labeling’ as: ‘all labels and other written, printed, or graphic matter (1) upon any article or any of its containers or wrappers, or (2) accompanying such article’ at any time while a device is held for sale after shipment or delivery for shipment in interstate commerce. The term ‘accompanying’ is interpreted liberally to mean more than physical association with the product. It extends to posters, tags, pamphlets, circulars, booklets, brochures, instruction books, direction sheets, fillers, etc. ‘Accompanying’ also includes labeling that is brought together with the device after shipment or delivery for shipment in interstate commerce. According to an appellate court decision: “Most, if not all advertising, is labeling.

The term ‘labeling’ is defined in the FFDCA as including all printed matter accompanying any article. Almost every country uses labeling-related requirements to help assure that devices are used safely and effectively. Such labeling may include adequate instructions for use, servicing instructions, adequate warnings against uses that may be dangerous to health, or information that may be necessary for the protection of users. Such notice, description or instruction can contain at least one selected from the group (but not limited to it) or any combination of it: The name and/or place of business of manufacturer, packer, or distributor optionally including the street address, city, state, and zip code. If the firm listed on the label is not the manufacturer, the firm information can be qualified by an appropriate statement such as, “Manufactured for . . . ” or or “Distributed by . . . ”. If a packer, distributor, or seller intends a device for uses other than those intended by the person from whom he received the device, these parties can furnish adequate labeling in accordance with the new intended use.

If a manufacturer knows or has information indicating that his device is to be used for conditions or purposes other than which it was intended, he can provide adequate labeling in accordance with such other uses. (An example of this might be a manufacturer of dental X-ray equipment who is routinely selling his product to podiatrists) “Adequate directions for use” means directions under which the layman can use a device safely and for the purposes intended. This can include —Statements of all purposes for which and conditions under which the device can be used; Quantity of dose for each use and usual quantities for persons of different ages and physical conditions; Frequency of administration; Duration of application; Time of administration in relation to other factors;

The notice, description or instruction, including but not limited to labeling means (e.g., treatment and/or operation guidelines) can be provided in any form that conveys the requisite information. Instruction means can be audio, for example spoken word, recorded in analog or digital form (e.g., audio recording), or received and/or transmitted in analog or digital form (e.g., by telephone, conference call, or audio signal transmitted over a network). Such information can also be visual or video, for example hard-copy (e.g., as a manual, recorded medium, booklet, leaflet, book and the like) or soft-copy (e.g., recorded in analog or digital form as a file recorded on an magnit, electronic, optical, or computer readable medium such as a DVD, disk drive, CD-ROM and the like). Additionally, instruction means can be interactive or real-time (e.g., a teleconference or internet chat or chat bot).

Some mediums, kits, devices or agents of this invention can include printed or made in any other way instructions to inform the user of the steps required to properly use it, optionally for reduction, deactivation, inhibition or degradation of Target and/or, optionally, for anti-aging treatment.

In some embodiments, an mediums, kits, devices or agents of this invention include a label configured to be coupled to respective mediums, kits, devices or agents of this invention. The label includes a first surface and a second surface. In some embodiments, the first surface can be coupled to an outer surface of mediums, kits, devices or agents of this invention. In some embodiments, for example, the first surface can include an adhesive. The second surface can include a textual indicia, such as, for example, a description of the mediums, kits, devices or agents of this invention, a mark indicating its manufacturer or distributor and/or an instruction associated with the use of such mediums, kits, devices or agents of this invention. The label can further include an electronic circuit system configured to output an electronic signal. In some embodiments, the electronic signal can include an instruction associated with the use of the mediums, kits, devices or agents of this invention.

In some embodiments the instruction is an instruction for use as medical device. In some embodiments the instruction is an instruction for use as medication.

In some embodiments, the notice, description or instruction, including but not limited to labeling can be shown on the lenses, computer glasses, transmitted via brain computer interface or by any other means or can be encoded by the Quick Response Code or any other machine readable form.

The notice, description or instruction, including but not limited to labeling can be implemented in digital electronic circuitry, or in computer firmware, hardware, software, or in combinations thereof. The implementation can be as a computer program product, e.g., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be recorded in any form of programming language, including compiled or interpreted languages, and the computer program can be deployed in any form, including as a stand-alone program or as a subroutine, element, or other unit suitable for use in a computing environment. A computer program can be deployed to be executed on one computer or on multiple computers at one site or several sites.

The notice, description or instruction, including but not limited to labeling can be performed by one or more programmable processors executing a computer program to perform functions of the invention by operating on input data and generating output. It can also be performed by, and an apparatus can be implemented as, special purpose logic circuitry, e.g., an FPGA (field programmable gate array) or an ASIC (application-specific integrated circuit). Subroutines can refer to portions of the computer program and/or the processor/special circuitry that implements that functionality.

In some embodiments, kit of this invention further comprises information about approval by the relevant agency of manufacture, use or sale for human administration.

The non-limiting examples of kits of this invention, could be paper kits which are the paper boxes, comprising corresponding pharmaceutical composition or device described in this disclosure, paper instruction and description, comprising name of intervention, indication and instruction.

| | Agent/Mechanism of | | |
|---|---|---|---|
| | Action/Mode of | | |
| Name | action | Indication | Extract from instruction |
| ASD1 | anti-GDF15 antibody | anti-aging, anti-frailty, | For intravenous injections, |
| | | amelioration of moderate | one vial per injection, once |
| | | cognitive decline, | every 2 months. |
| | | amelioration hand grip | |
| | | strength lose and | |
| | | amelioration of other age | |
| | | related deficits | |
| GER23 | peptide inhibitor of | anti-aging, anti-frailty, | For intravenous injections, |
| | FRZB | amelioration of moderate | one vial per injection, once |
| | | cognitive decline, | every 8 months. |
| | | amelioration hand grip | |
| | | strength lose and | |
| | | amelioration of other age | |
| | | related deficits | |
| GER56 | *Gero sorption | anti-aging, anti-frailty, | For extracorporeal sorption |
| | column (selectively | amelioration of moderate | of proteins GDF15, FSTL3, |
| | reduces GDF15, | cognitive decline, | BMP4 and FRZB from |
| | FSTL3, BMP4 and | amelioration hand grip | circulating blood. Gero |
| | FRZB in blood | strength lose and | sorption column should be |
| | plasma) | amelioration of other age | used only under the care of |
| | | related deficits | a physician experienced in |
| | | | apheresys. Patient |
| | | | connected to the |
| | | | extracorporeal system must |
| | | | be supervised at all times |
| | | | by a physician or qualified |
| | | | health care professional. |
| FDS89 | small molecule | anti-aging, anti-frailty, | For intravenous injections, |
| | inhibitor of BMP4 | amelioration of moderate | one vial per injection, once |
| | | cognitive decline, | every 12 months. |
| | | amelioration hand grip | |
| | | strength lose and | |
| | | amelioration of other age | |
| | | related deficits | |
| NP882 | Anti-GDF15 | anti-aging, anti-frailty, | For intravenous injections, |
| | Nanoparticle | amelioration of moderate | one vial per injection, once |
| | | cognitive decline, | every 24 months. |
| | | amelioration hand grip | |
| | | strength lose and | |
| | | amelioration of other age | |
| | | related deficits | |

In some embodiment this invention is a method, including but not limited to the method of anti-aging treatment, comprising administering by the subject at least one of the compositions, molecules or other agents described in this disclosure, including but not limited to binding elements, optionally in therapeutically effective amount. In some embodiments this invention is a method, comprising administering to the subject an effective amount of molecule selected from the group: monoclonal or polyclonal antibody, protein, aptamer, peptide, polymer, virus or small molecule or any other binding element, capable of inhibiting an activity of at least one of the Targets circulating in blood plasma or any combination of such Targets and/or binding to such Target. In some embodiments this invention is a method of treatment, wherein an molecule for administration for respective Target is selected from binding elements described in this application or is its analog, prodrug or derivative.

In some embodiment this invention is a method of treatment of this disclosure, wherein the median difference in percentage in concentrations of components shown in “Biomarkers” table in plasma infused into subject versus plasma extracted from the subject is at least 2 fold less or significantly less than the difference in concentration of Target in input and output plasma.

In some embodiment this invention is a method of treatment, optionally anti-aging treatment, comprising reduction of one or several Targets in blood stream of the subject, optionally by therapeutic mean, including but not limited to by protein binding or filtering device, wherein the median difference in percentage in concentrations of at least 10%, 25%, 50%, 75%, 85%, 90%, 95% of at least 3000 proteins of blood plasma in plasma infused into subject versus plasma extracted from the subject is at least 2 fold less or significantly less than the difference in concentration of Target in input and output plasma.

In some embodiments this invention is a method of treatment of at least one of the disease or condition or decline mediated by at least one of the proteins selected from the Targets, comprising deactivation or reducing or inhibition or degrading or binding of such Target in blood plasma, optionally wherein such deactivation or reducing or inhibition or degrading or binding is achieved by therapeutic mean.

In some embodiments this invention is a method of treatment, including but not limited to anti-aging treatment comprising administering to the subject an effective amount of an antigen binding protein capable of inhibiting an activity of at least one of the Targets circulating in blood plasma or any combination of such Targets.

In some embodiments this invention is a method of treatment, including but not limited to anti-aging treatment, comprising step of administering by the subject of agent, deactivating or binding or inhibiting or degrading a respective Target or deactivating or binding or inhibiting or degrading or activating at least one effector upstream or downstream of at least one of the Targets what has anti-aging effect, including but not limited at least one agent described in this disclosure, including but not limited to agent selected from the group: monoclonal antibody, polyclonal antibody, fAb, protein, aptamer, peptide, polymer, virus or small molecule or at least one of the agents selected from the binding elements described in this application or is its analog.

In some embodiments, this disclosure is a method of deactivation, removing, degradation, deleting or inhibiting the Target circulating in blood, optionally selective, by any mean. In some embodiments such method is a method of anti-aging treatment, wherein the Target is deactivated, removed, degraded, deleted or inhibited in a therapeutically acceptable way, including but not limited to—in effective and safe amounts and regimen. In some embodiments this invention is a method of treatment comprising step of deactivation or reduction or deletion or binding or inhibition or degradation in blood plasma of patient of at least one of the proteins selected from the Targets or combinations thereof inside the blood circulation of the subject or extracorporeally-with further infusion of extracorporeally treated plasma back into subject. In some embodiment such method is implemented by the reduction of respective Target by the means of sorption device or protein binding device or by sorption. In some embodiments such method is implemented by using device of this invention, optionally with at least one of the binding elements or any combination of binding elements described in this disclosure.

In some embodiments this invention is a method, including but not limited to method of anti-aging treatment comprising steps: Extraction of blood plasma from the patient, Treating such plasma extracorporeally by the agent selected from the group: monoclonal or polyclonal antibody, optionally humanized, fAb, protein, aptamer, peptide, polymer, virus, nanoparticle, or small molecule or any other object, binding or capturing a respective Target or the complex of such agent with magnet or any other object facilitating extraction of complex of agent with the Target from the plasma, Extraction of the complex of agent and Target from the plasma, Returning the treated plasma back into patient. In of the examples of magnetic separations of Targets shown in in Example 25.

In some embodiments this invention is a method described in this disclosure or alike, wherein treated plasma of one individual is infused into the other patient's circulation.

In some embodiments this invention is a use of device, including but not limited to protein binding or filtering device for decreasing proteins selected of Targets in blood plasma. In some embodiments this invention is a use of device, including but not limited to protein binding or filtering device for decreasing proteins selected of Targets in blood plasma for the treatment, including but not limited to the anti-aging treatment.

In some embodiments this invention is a method of obtaining plasma product, comprising a step of passing plasma of the patient over the device, comprising binding element to at least one protein selected from the group Targets under conditions which effect the binding of said binding element to corresponding Target in plasma, thereby removing a significant portion of the corresponding Target from the plasma, optionally selectively removing corresponding Target from the plasma, optionally reducing Target to Negligible amount in one or more rounds of plasma passing over the device.

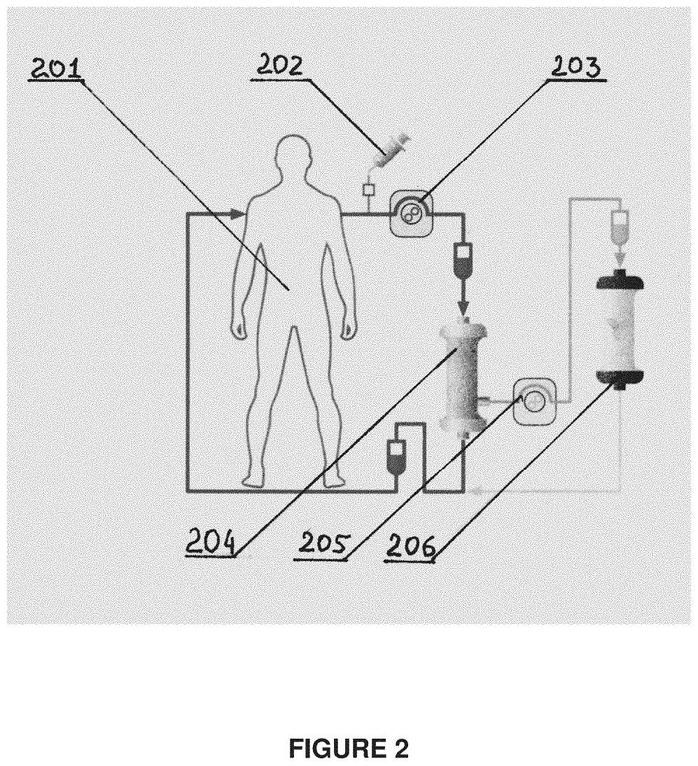

In some embodiments this invention, including but not limited to devices, kits, methods and uses relate to apheresis procedure or can be implemented in such procedure (For example as shown on FIG. 2). Apheresis is an extracorporeal medical procedure that involves removing whole blood from a patient 200 (and separating blood into individual components so that one particular component can be either removed or exposed to various manipulations ex vivo (outside of the human body), e.g. by separator 201. The remaining blood components as well as those changed (e.g. purified from the particular Targets by protein binding device 203) are then returned back into bloodstream of the same patient. Therapeutic apheresis includes a number of procedures with the aim to treat certain medical conditions in which a blood component that contains disease-provoking elements or the elements themselves are removed. Conventional apheresis techniques include plasmapheresis and cytapheresis.

Selective sorption/adsorption is an extracorporeal plasma purification medical procedure which allows to eliminate targeted plasma circulating components. It can be performed with the special medical device, which separates the blood components and has a replaceable adsorption column for plasma purification. This column can contain a sorber or sorbent with binding elements of different nature, which core function is to provide selective elimination of Target or Targets.

The first step of the procedure can be a standard apheresis providing whole blood separation into blood cells and plasma. The patient's vein can be catheterized and the blood is directed to a machine via tubes. The separation can be done by either centrifuge or filtration process in the machine or in any other acceptable way. In preferred embodiment, proper anticoagulation therapy must be provided according to the current standards of care. Separated plasma can be further directed into the adsorption column. The elimination dose can be determined by the plasma volume pumped through the adsorption column. In some cases double interchangeable columns may be used in frames of a single procedure: at any time instance one is being used while another is being purified by the buffer solution. Purified plasma is then re-united with the blood cells and reinfused back to the bloodstream. The entire procedure usually takes several hours, depending on the volume of blood being treated.

Selectivity of Target reduction can be achieved due to the binding elements' or filter's or other capture agent's nature. Some methods to achieve selectivity of binding elements is described in this disclosure. Binding elements may include small molecules, antibodies (mono- or polyclonal), peptides, nucleic acids (aptamers), nanoparticles and other molecules and elements, including but not limited to those described in this disclosure.

Current indications for therapeutic apheresis include over 100 diseases with different degrees of clinical evidence, mostly toxic and acute conditions. The procedure as a rule is contradicted to the patients with low protein level in plasma (hypoproteinemia), bleeding disorder, low blood pressure, active infections, individual hypersensitivity to the components of the device which interact with blood or its components. More details on procedures related to the use of protein binding amd alike devices can be found in the art, including but not limited to Okafor C, et al. J Clin Apheresis, 25:240-249, 2010, Schwartz at al. Journal of Clinical Apheresis 31:149-162 (2016).

The dosage levels and mode of administration can be dependent on a variety of factors such as the treatment used, the device, the active agent, the context of use (e.g., the patient to be treated), and the like. Optimization of modes of administration, dosage levels, and adjustment of protocols, including monitoring systems to assess effectiveness are routine matters well within ordinary skill. In some embodiments, optimization of modes of administration, dosage levels, and adjustment of protocols etc. are designed to keep Target concentration in negligible amount or less most of the time, or for at about 1 month, or from 1 months to 6 months, or from 6 months to 12 months, or from 12 months to 24 months, or from 24 months to 48 months, or for up to 5 years, or for up to 10 years, or from about 1 month to about 10 years, or for more then 10 years, or for as long as possible, or for lifelong or for other period defined by doctor or patient.

Further discussion of optimization of dosage and treatment regimens can be found in Benet et al., in Goodman & Gilman's The Pharmacological Basis of Therapeutics, Ninth Edition, Hardman et al., Eds., McGraw-Hill, New York, (1996), Chapter 1, pp. 3-27, and L. A. Bauer, in Pharmacotherapy, A Pathophysiologic Approach, Fourth Edition, DiPiro et al., Eds., Appleton & Lange, Stamford, Conn., (1999), Chapter 3, pp. 21-43, and the references cited therein, to which the reader is referred.

In some embodiments, use of the device of any of any one of the claims for blood purification, reduction of specific Target and method of reduction of specific protein by such devices is an ex-vivo method and use to produce purified blood.

In some embodiments this invention is a use of a any ligand or binding element for protein selected from the Targets or their analog or their complex with at least one of the linkers in the manufacture of a protein binding device having said ligand or other object described above coupled thereto, optionally, for the anti-aging treatment of a patient, said treatment comprising passing plasma of the patient over the device under conditions which effect the binding of said specific ligand to corresponding Target in the patient's plasma, thereby removing a significant portion of the corresponding Target from the patient's plasma, and reinfusing the plasma to the patient.

In some embodiments this invention is a use of binding element of at least one protein selected from the Targets for manufacturing of protein binding device or sorbent, optionally wherein such device or sorbent is for treatment, optionally for anti-aging treatment.

In some embodiments this invention is a use described in this disclosure of binding element of at least one of the Targets, wherein binding element is monoclonal or polyclonal antibody, optionally humanized, protein, aptamer, peptide, polymer, virus, nanoparticle or small molecule, binding a respective Target or at least one of the binding elements described in this disclosure, including but not limited to wherein binding element is selected for respective Target from binding elements described in this disclosure is its structural or functional analog.

In some embodiments this invention is a use of binding element of at least one protein selected from the Targets in sorption device or sorbent, wherein such binding element is linked to such protein binding device or stationary phase, optionally wherein protein binding device or stationary phase is for use in treatment, optionally—anti-aging treatment. In some embodiments this invention is a use of at least one of the binding elements described in this disclosure as a binding element in protein binding device or stationary phase for treatment, optionally—anti-aging treatment.

A non-limiting example of testing the stability and Reproducibility of the protein binding device described in this disclosure is shown in Example 8.

In some embodiments, the disclosed device, kit, medication, agent, binding element, pharmaceutical composition or method is a treatment, optionally an anti-aging treatment and or is for treatment, optionally—anti-aging treatment.

In some embodiments, this invention is a disclosed device, kit, medication, agent, binding element, pharmaceutical composition or method wherein the binding element or therapeutic agent is an antibody which is humanized using at least one antibody described in this disclosure as a binding element or human antibody made using at least one antibody described in this disclosure as a binding element.

In some embodiments, the binding element or other agent or intervention degrades the Target by enhancing the rate of the clearance the Target from the systemic circulation.

In some embodiments, device, kit and method suggested can remove at least one Target and at least one of some other pro-aging or otherwise harmful elements from the plasma to provide additional benefit to patient from the procedure.

For every skilled in the art it is obvious that the therapeutic anti-aging effect will be achieved irrespectively of the nature of deletion or degrading the Targets in plasma extracorporeally or intracorporeally and the means by which such deletion or degrading is achieved given acceptable level of side effects related to the particular mean is reasonably preserved. The present disclosure covers any and all such deletion or degrading.

It is also obvious that the therapeutic anti-aging effect will be achieved irrespectively of the nature and the mean of binding, inhibition or deactivation of Target whether directly or indirectly. In best case scenario binding, inhibition or deactivation should mimic the removal in a sense that all of considerable part of at least one of Targets contained in plasma should be permanently or for along time bound, inhibited or deactivated. There are numerous ways to achieve binding, inhibition or deactivation, e.g. by medication, including but not limited to gene therapy, a monoclonal or polyclonal antibody, optionally humanized, which recognizes at least one respective Target, protein, aptamer, peptide, polymer, virus or small molecule, vaccine, nanoparticle or any other therapeutic agent. The present disclosure covers any and all such binding, inhibition and deactivation.

In some embodiments, this invention is made to produce an additive effect of deletion, reduction, binding, inhibiting or degrading at least two or more of different Targets, and comprises the corresponding devices, agent, combination of therapeutic agents and methods of deletion, deactivation, reduction, binding, inhibiting or degrading at least two or more of different Targets, including but not limited to devices, agents and methods described in this disclosure and modified to produce such additive effect. Non-limiting example of such therapy for additive effect could be e.g. combination therapy comprising anti-GDF15 and anti-BMP4 antibodies, method to reduce FRZB and GDF15 in blood stream of the subject for anti-aging treatment in one procedure or with the interruption not more than 8 weeks, or not more than 1 year etc.

In some embodiments, this disclosure provides a protein binding device, wherein the protein binding device the comprises a binding elements that selectively bind protein selected from: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB. In some embodiments, such device comprises at least one of the binding elements described in this application or the molecule comprising the part binding such Target at least 99% structurally similar, at least 95% structurally similar, at least 90% structurally similar, or at least 80% structurally similar, or at least 70% structurally similar to the part binding such protein of at least one of the binding elements described in this application.

In some embodiments, this disclosure provides a protein binding device which comprises the binding element that binds to at least one effector upstream or downstream of at least one of the protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB, optionally wherein an effect of reduction of such protein or element mimics at least one of the following a reduction or deletion or binding or inhibition or degradation in blood plasma of patient of at least one of the proteins selected from the Targets or has similar biological effect or such reduction has an anti-aging effect.

In some embodiments, this disclosure provides a protein filtering device that filters out or, optionally, selectively filters out a secondary protein or at least one other at least one effector upstream or downstream of at least one of protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB, optionally wherein an effect of reduction of such protein or element mimics at least one of the following: a reduction or deletion or binding or inhibition or degradation in blood plasma of patient of at least one of the proteins selected from the Targets or has similar biological effect or such reduction has an anti-aging effect.

In some embodiments, the device comprises any mean to bind or capture agent to at least one of the Targets for example from the fluid contacting with such device or passing through the such device or housing of such device. The binding or capturing can be achieved in the numerous ways. It is obvious for the expert in the field that once the Target, which should be removed from the blood to achieve the therapeutic effect, is known it is generally does not matter by which particular method it will be removed.

For every skilled in the art it is obvious that the Targets can be removed and therapeutic anti-aging effect will be achieved irrespectively of the nature of binding or capturing of the corresponding Target in extracorporeal sorption or filter or other respective device and the structure or material of the binding element or capture agent. The present disclosure covers any and all such binding and capturing and other methods to remove Target and corresponding devices and systems. For example, such binding element can be a small molecule, protein, antibody, peptide, aptamer, polymer or any other molecule or any other object that selectively binds Target.

For every Target binding elements can be designed or can just be obtained from the shelf as shown in this disclosure, including but not limited those described as binding elements in this application. At the modern level of the technology development there are numerous means and ways to achieve such removal applicable for extracorporeal therapy. The non-limiting examples of such means could be molecules (binding elements, e.g. antibodies, aptamers etc.) that are linked to the stationary phase of such device (e.g. sorbent in sorption column) and bind at least one of the Targets, it could also be some binding elements which can be extracted together with bound Target by any way (e.g. but not limited to magnets, nanoparticles that caught Target protein etc.), it could be materials that due to some reason capture Target and optionally let the most of other elements of plasma passing through it go uncaptured (e.g. special filter having structure that captures Target and optionally allows most other plasma elements go uncaptured, it could also be some force that captures Target and optionally allows the most of other elements of plasma passing through it go uncaptured and many other means to remove the Target).

The identification of binding elements can be done in many ways. Given that for binding elements for sorption columns there are substantially less requirements than for medication (since there are less or no ADMET properties requirements) identifying or obtaining a new binding element is not a challenging task and can be accomplished in many different ways using standard procedures well known to the skilled in the art. In preferred embodiment, binding element should have at least “some” binding to the chosen protein Target—as a rule the best case is to have strong binding elements with picomolar or nanomolar range binding to the Target, but hundreds of micromolar binding most likely also be acceptable due to the nature of the sorption column or other sorption device—if the binding is not strong enough to extract the needed amount of the Target from the blood plasma in the acceptable time one should just increase the amount of same binding elements in the column or increase the duration of the sorption procedure or both so the weak binding will be compensated.

In some embodiment, there should be “some” selectivity of binding element towards selected Target. The selectivity can be checked trivially after the binding element is identified by measuring how many non-Target elements are bound by the binding element. In many cases as a rule of thumb the less non-Target elements are bound by the binding element the less likely the use of such binding element in sorption column will cause any substantial harm to the patient. In some preferred embodiments, the binding element should bind just a selected Target and not anything else, besides cases when the single binding element is designed to bind few different Targets or Target and some other blood element intended to be reduced for any therapeutic purpose.

In some embodiments, the acceptable is also a situation that binding element binds some or many other elements in plasma but does not cause the critical adverse effects doing it, making risk/benefit ratio unacceptable. In majority of the cases using of the binding element to any of the Targets in the column will not cause serious side and toxic effects, since even taking out a big amount of plasma with all proteins contained in it from the patient is well tolerated so the taking out some other plasma elements together with the Target protein most likely will not cause any substantial harm comparable with the danger of aging and age related morbidity. Plasmapheresis is frequently used to collect source plasma that is used for manufacturing into medications. For example, plasmapheresis donors in the United States are allowed to donate large volumes twice a week and could nominally donate 83 liters (about 22 gallons) in a year. One of the many possible ways to check selectivity of binding element is described in Example 3.

Selectivity. In the case of non-ideal selectivity in many embodiments the binding element should still bind the selected Target in preferred embodiment—at the level on which the fact that binding element binds other things should not result in the need to put too much binding element into device or to have a very long procedure of using the column with the binding element just to keep the cost of the column with such amount of binding element, the size of the column and the duration of the procedure reasonable. These “reasonability” requirement is soft, because one can still produce of sorption column breaking this reasonability requirement that still will have a desired therapeutic effect when used.

Since the plasma is streamed through the sorption column and as a rule binding elements are linked to the sorbent, plasma goes through, in many embodiments, the binding element should have stability in blood plasma enough to be functional at least one procedure during which it should bind the Targets in plasma and stay in sorption column with the Target bound to it until the procedure ends. Generally such stability is easily achievable since when the binding element of any nature is linked to the sorbent by any way in most cases it possess the acceptable stability. The plasma stability can easily be checked after the candidate binding element is identified or obtained by the methods known in the art. In this disclosure there are several non-limiting examples how the binding elements to the Targets can be obtained and used in sorption device are listed.

In some embodiments, the protein binding device comprises binding element which is a polyclonal antibody, which recognizes at least one protein selected from the Targets. In some other embodiments this invention is a pharmaceutical composition comprising a polyclonal antibody, which recognizes at least one protein selected from the Targets. Polyclonal antibodies bind to multiple epitopes (the part of an antigen that is recognized by the antibody) and are usually made by several different plasma cell (antibody secreting immune cell) lineages. Bispecific monoclonal antibodies can also be engineered, by increasing the therapeutic Targets of one single monoclonal antibody to two epitopes.

Antibodies of this invention may exist in a variety of other forms including, for example, Fv, Fab, and (Fab′)2, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., Immunology, Benjamin, N. Y., 2nd ed. (1984), and Hunkapiller and Hood, Nature, 323, 15-16 (1986).

Polyclonal antibodies are easily obtainable in any reasonable amount by standard procedures, it is obvious for any skilled in the art that for every Target it is trivial to obtain polyclonal antibodies in any reasonable amount either by just buying it from the shelf as those shown in this disclosure, including but not limited to those described as binding element in this application or by immunization procedure. A non-limiting example of method of obtaining polyclonal antibodies is shown in Example 4.

The obtained antibody can further be linked to stationary phase (for example to sorbent for medical device or to special agent e.g. those that further can be used to extract such antibody complex with the bound Target by magnet or any other force or to any other agent facilitating removal of Target from plasma or to nanoparticle etc.). There are many methods how to link antibody to the stationary phase are known in the art including but not limited as shown in Example 5.

Various examples of polyclonal antibodies which are binding elements for respective Targets are shown in this disclosure.

In some embodiments, the disclosed protein binding device comprises binding element, which is a monoclonal antibody. In some embodiments, the disclosed agent or pharmaceutical composition is or comprises monoclonal antibody. Monoclonal antibodies (mAb or moAb) are antibodies that are made by identical immune cells that are all clones of a unique parent cell. Monoclonal antibodies can have monovalent affinity, in that they bind to the same epitope. Given almost any substance, it is possible to produce monoclonal antibodies that specifically bind to that substance.

The antigen binding proteins that are provided include monoclonal antibodies that bind to Target to various degrees. Monoclonal antibodies can be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

In some instances, a hybridoma cell line is produced by immunizing an animal (e.g., a transgenic animal having human immunoglobulin sequences) with an immunogen comprising (1) full length human Target, obtained by transfecting CHO cells with cDNA encoding a human full length Target; harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds to Target. Such hybridoma cell lines, and the monoclonal antibodies produced by them, form aspects of the present disclosure. Monoclonal antibodies secreted by a hybridoma cell line can be purified using any technique known in the art. Hybridomas or mAbs can be further screened to identify mAbs with particular desired properties, such as the ability to bind the Target, inhibit Target activity and/or signaling. Additional information on methods of making and using polyclonal and monoclonal antibodies are described, e.g., in Harlow et al., Using Antibodies: A Laboratory Manual: Portable Protocol I. Cold Spring Harbor Laboratory (Dec. 1, 1998), which is incorporated by reference in its entirety. Methods for making modified antibodies and antibody fragments (e.g., chimeric antibodies, reshaped antibodies, humanized antibodies, or fragments thereof, e.g., Fab′, Fab, F(ab′)2 fragments); or biosynthetic antibodies (e.g., single chain antibodies, single domain antibodies (DABs), Fv, single chain Fv (scFv), and the like), are known in the art and can be found, e.g., in Zola, Monoclonal Antibodies: Preparation and Use of Monoclonal Antibodies and Engineered Antibody Derivatives, Springer Verlag (Dec. 15, 2000; 1st edition), which is incorporated by reference in its entirety.

Antibodies targeting other proteins (non-Targets) were used in therapeutic sorption, for example Therasorb® immunoadsorption (IA) of Plasmaselect, Teterow, Germany, by selectively eliminating pathogenic substances from the circulation, allows for successful therapy of previously insufficiently treatable diseases. Molecules (specific polyclonal sheep antibodies) coupled to a matrix (Sepharose CL 4B) selectively bind plasma components in extracorporeal circulation. This procedure has been established in the treatment of various diseases. Examples are familial hypercholesterolemia (LDL-Therasorb®) and selected autoimmune diseases (Ig-Therasorb®). In some embodiment this invention can be an analog of Therasorb®, LDL-Therasorb® or Ig-Therasorb, wherein instead of antibodies used in these devices, the antibodies (or other binding elements) to at least one of the Target is used, wherein all other features are generally preserved, including but not limited to the manufacturing procedure, composition etc.

Instead of antibodies (polyclonal or monoclonal) the immunologically functional fragment can be used to bind Target. The term “immunologically functional fragment” (or simply “fragment”) of an Target binding protein, e.g., immunoglobulin chain (heavy or light chain) or an antibody, as used herein, is a Target binding protein comprising a portion (regardless of how that portion is obtained or manufactured) of an antibody that lacks at least some of the amino acids present in a full-length chain but which is capable of specifically binding to Target. Such fragments are biologically active in that they bind specifically to Target and can compete with other Target binding proteins, including intact antibodies, for specific binding to a given epitope. In one aspect, such a fragment will retain at least one CDR present in the full-length light or heavy chain, and in some embodiments will comprise a single heavy chain and/or light chain or portion thereof. These biologically active fragments can be produced by recombinant DNA techniques, or can be produced by enzymatic or chemical cleavage of antigen binding proteins, including intact antibodies. Immunologically functional immunoglobulin fragments include, but are not limited to, Fab, Fab′, F(ab′)2, Fv, domain antibodies and single-chain antibodies, and can be derived from any mammalian source, including but not limited to human, mouse, rat, camelid or rabbit. It is contemplated further that a antibody or functional portion of the Target binding proteins described herein, for example, one or more CDRs, could be covalently bound to a second protein or to a small molecule to create a therapeutic agent directed to Target, possessing bifunctional therapeutic properties, or having a prolonged serum half-life.

It is possible to create fully human antibodies in mice. Fully human mAbs lacking any non-human sequences can be prepared from human immunoglobulin transgenic mice by methods known in the art, including but not limited to Lonberg et al., NATURE 368:856-859, 1994; Fishwild et al., NATURE BIOTECHNOLOGY 14:845-851, 1996; and Mendez et al., NATURE GENETICS 15:146-156, 1997.

The human antibody can also be obtained and optimized from phage display libraries by techniques described in, e.g., Knappik et al., J. MOL. BIOL. 296:57-86, 2000; and Krebs et al., J. Immunol. Meth. 254:67-84 2001) and other ways known in the art and yet to be introduced.

The monoclonal antibody can optionally be humanized. The humanized antibody preferably shall have the same affinity for the Target as the non-humanized antibody from which it was derived. Humanization can be performed, for example, using various methods by substituting at least a portion of a rodent variable region for the corresponding regions of a human antibody (See e.g., Jones et al., (1986) Nature 321:522-525; Riechmann et al., (1988) Nature 332:323-27; Verhoeyen et al., (1988) Science 239:1534-1536).

The humanization can be done for example when chimeric proteins can be created in which mouse immunoglobulin constant regions are replaced with human immunoglobulin constant regions. See, e.g., U.S. Pat. No. 4,816,567 (Cabilly), Morrison et al., 1984, PROC. NAT. ACAD. SCI. 81:6851-6855, Neuberger et al., 1984, NATURE 312:604-608; U.S. Pat. No. 5,500,362 (Robinson) and U.S. Pat. No. 6,893,625 (Robinson).

Another one approach to humanization is CDR grafting. In this approach the CDRs of the light and heavy chain variable regions are grafted into frameworks from another species. For example, murine CDRs can be grafted into human FRs. In some embodiments, the CDRs of the light and heavy chain variable regions of an anti-Target antibody are grafted into human FRs or consensus human FRs. To obtain consensus human FRs, FRs from several human heavy chain or light chain amino acid sequences are aligned to identify a consensus amino acid sequence. CDR grafting is well known in the art and can be found for example in U.S. Pat. No. 6,982,321 (Winter); U.S. Pat. No. 7,022,500 (Queen); U.S. Pat. No. 5,693,762 (Queen); U.S. Pat. No. 6,180,370 (Queen); U.S. Pat. No. 6,054,297 (Carter); U.S. Pat. No. 5,859,205 (Adair); U.S. Pat. No. 5,693,761 (Queen); U.S. Pat. No. 5,585,089 (Queen); U.S. Pat. No. 5,530,101 (Queen); U.S. Pat. No. 5,565,332 (Hoogenboom); Jones et al., 1986, NATURE 321: 522-525; Riechmann et al., 1988, NATURE 332: 323-327; Verhoeyen et al., 1988, SCIENCE 239: 1534-1536; and Winter, 1998, FEBS LETT 430: 92-94.

In another one approach known as “SUPERHUMANIZATION™,” human CDR sequences are chosen from human germline genes, based on the structural similarity of the human CDRs to those of the mouse antibody to be humanized. The approach is known in the art and can be found for example in U.S. Pat. No. 6,881,557 (Foote); and Tan et al., 2002, J. IMMUNOL. 169:1119-1125.

Some of other methods to decrease immunogenicity are known as “reshaping,” “hyperchimerization,” and “veneering/resurfacing.” See, e.g., U.S. Pat. No. 6,072,035 (Hardman) and Vaswami et al., 1998, ANNALS OF ALLERGY, ASTHMA, & IMMUNOL. 81:105; Roguska et al., 1996, PROT. ENGINEER 9:895-904 and. In the veneering/resurfacing approach, the surface accessible amino acid residues in the murine antibody can be replaced by amino acid residues more frequently found at the same positions in a human antibody. This type of antibody resurfacing is described, e.g., in U.S. Pat. No. 5,639,641 (Pedersen).

One more technology for transforming a mouse antibody into a form acceptable for human medical is known as ACTIVMAB™ (Vaccinex, Inc., Rochester, N.Y.), which involves a vaccinia virus-based vector to express antibodies in mammalian cells described in for example U.S. Pat. No. 6,706,477 (Zauderer); U.S. Pat. No. 6,800,442 (Zauderer); and U.S. Pat. No. 6,872,518 (Zauderer).

The example of another one technology for modifying a mouse antibody into a form acceptable for use in humans is technology practiced by KaloBios Pharmaceuticals, Inc. (Palo Alto, Calif.). This technology involves the use of a proprietary human “acceptor” library to produce an “epitope focused” library for antibody selection.

Another approach for converting a mouse antibody into a form suitable for medical use in humans is known as HUMAN ENGINEERING™ technology, which is practiced by XOMA (US) LLC. See, e.g., PCT Publication No. WO 93/11794 and U.S. Pat. No. 5,766,886 (Studnicka); U.S. Pat. No. 5,770,196 (Studnicka); U.S. Pat. No. 5,821,123 (Studnicka); and U.S. Pat. No. 5,869,619 (Studnicka).

Any suitable method, including but not limited to any of the above techniques, can be used to reduce or eliminate human immunogenicity of an antibody.

The antibody binding element can be linked to the device or stationary phase by many ways known in the art and newly developed, including but not limited to as shown in this disclosure.

In some embodiments, the disclosed protein binding device comprises binding element, which is a protein. One of the examples of use of proteins as a binding elements for non-Targets proteins in sorption devices is protein A. For example The Prosorba® Column is a plasma filtering device used to treat severe cases of rheumatoid arthritis or psoriatic arthritis. Its active element is Protein A bonded to a diatomaceous earth/clay bead. Another example of protein A use as a binding element is Immunosorba® column. The effect of the Protein A is to remove circulating immune complexes responsible for the autoimmune joint deterioration process. Some other proteins can be used as binding elements for Targets. Some proteins described in this application as binding elements are known in the art to be binding elements of Targets and can be used as binding elements, other protein binding elements for Targets can be found by the methods known in the art. The development of the proteins can be made by methods known in the art, one of the possible ways to manufacture protein binding element is described in Example 6 “Recombinant protein production”. The purification of proteins can be made by the methods known in the art, e.g as described in GE Healthcare Life Sciences “Recombinant Protein Purification Principles and Methods” 18-1142-75 AE or applicable regulations related to medication or medical device production.

The protein binding elements to be used with device or stationary phase should be linked to it, the linking of proteins to device or stationary phase can be done in many ways known in the art, including but not limited to as shown in this disclosure as in Example 5.

In some embodiment the device of this invention can be an analog of Immunosorba® column, wherein instead of protein A used in Immunosorba®, at least one of the proteins which is a binding element to at least one of the Target (or other binding elements) can be used, and wherein all other features are generally preserved, including but not limited to the manufacturing procedure, composition etc.

In some embodiments, the disclosed protein binding device comprises binding element which is an aptamer or SOMAmer.

Aptamers are oligonucleotide or peptide molecules that bind to a specific Target. Aptamers are usually created by selecting them from alarge random sequence pool, but natural aptamers also exist in riboswitches. More specifically, aptamers that can be used as binding elements to Target can be classified as DNA or RNA or XNA aptamers, which consist of (usually short) strands of oligonucleotides. The other type of aptamers are peptide aptamers. They consist of one (or more) short variable peptide domains, attached at both ends to a protein scaffold.

Nucleic acid aptamers are nucleic acid species that can be engineered through repeated rounds of in vitro selection or equivalently, SELEX (systematic evolution of ligands by exponential enrichment) to bind to various Targets. Both DNA and RNA aptamers can show robust binding affinities for various Targets.

Smart aptamers also can be used as binding elements to at least one of the Targets. A concept of smart aptamers describes aptamers that are selected with pre-defined equilibrium, rate constants and thermodynamic (AH, AS) parameters of aptamer-Target interaction. Kinetic capillary electrophoresis is the technology used for the selection of smart aptamers. It obtains aptamers in a few rounds of selection. Kinetic capillary electrophoresis or KCE is capillary electrophoresis of molecules that interact during electrophoresis. Different KCE methods are designed by varying initial and boundary conditions—the way interacting molecules enter and exit the capillary. Several KCE methods are known: non-equilibrium capillary electrophoresis of the equilibrium mixtures (NECEEM), sweeping capillary electrophoresis (SweepCE), plug-plug KCE (ppKCE).

Peptide aptamers are artificial proteins selected or engineered to bind specific Target. These proteins consist of one or more peptide loops of variable sequence displayed by a protein scaffold. They are typically isolated from combinatorial libraries and often subsequently improved by directed mutation or rounds of variable region mutagenesis and selection. In vivo, peptide aptamers can bind cellular protein Targets and exert biological effects, including interference with the normal protein interactions of their Targeted molecules with other proteins. Libraries of peptide aptamers can be used as “mutagens”, in settings in which an investigator introduces alibrary that expresses different peptide aptamers into a cell population, selects for a desired phenotype, and identifies those aptamers that cause the phenotype. The researcher then uses those aptamers as baits, for example in yeast two-hybrid screens to identify the cellular proteins targeted by those aptamers. Such experiments identify particular proteins bound by the aptamers, and protein interactions that the aptamers disrupt, to cause the phenotype. Peptide aptamers can also recognize Targets in vitro.

Peptide aptamer selection can be made using different systems, but the most used currently is the yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers.

Aptamers have an innate ability to bind to any molecule they“re targeted at. Bound to a Target, as a rule aptamers inhibit its activity. Aptamers may suffer from two issues that limit their effectiveness. Firstly, the bonds they form with Target molecules are sometimes too weak to be effective, and second, they're easily digested by enzymes. Adding an unnatural base to a standard aptamer can increase its ability bind to Targets. A second addition in the form of a “mini hairpin DNA” gives the aptamer a stable and compact structure that is resistant to digestion, extending its life from hours to days. As a rule aptamers are less likely to provoke undesirable immune responses than antibodies.

Aptamers can be used as a binding elements in protein binding device or as therapeutic agents. One of the examples of aptamer use as a binding element in sorption device is described at (Wallukat G, Haberland A, Berg S, Schulz A, Freyse E J, Dahmen C, Kage A, Dandel M, Vetter R, Salzsieder E, Kreutz R, Schimke I. The first aptamer-apheresis column specifically for clearing blood of β1-receptor autoantibodies. Circ J. 2012; 76(10):2449-55). In some embodiments, a protein binding device of this invention using aptamers as binding elements can be an analog of the device described in this publication, wherein instead of aptamer used in the device described in publication, at least one of the aptamers or SOMAmers (including but not limited to those described in this invention) which is a binding element to at least one of the Target (or other binding elements) can be used, and wherein all other features are generally preserved as in publication, including but not limited to the manufacturing procedure, composition etc.

Examples of SOMAmer as a binding elements for Targets are shown in this disclosure. Like aptamers, SOMAmers are short, single-stranded deoxyoligonucleotides. They were selected in vitro from large libraries for their ability to bind to Targets. To give some of them greater Target diversity as well as high affinity, SomaLogic (Mailing Address:SomaLogic, Inc. 2945 Wilderness PI. Boulder, Colo. 80301 USA) scientists endowed them with protein-like properties by adding functional groups that mimic amino acid side chains, thereby expanding their chemical diversity. SOMAmer reagents are engineered with dU residues functionalized at the 5-position with different protein-like moieties (e.g., benzyl, 2-napthyl or 3-indolyl-carboxamide). These modifications can interact with more epitopes on a greater range of Target molecules, largely as a result of the novel secondary and tertiary structures formed within the SOMAmer reagent itself. SOMAmer reagents are selected not just for their affinity for their respective Target proteins, but also for low dissociation rates (slow off-rates) with their Targets. More details on SOMAmers can be found on somalogic.com, materials of SOMALOGIC, INC. 2945 Wilderness PI, Boulder, Colo. 80301, including but not limited to patent applications of Somalogic on SOMAmers. The binding to SOMAmers described in this disclosure to any of the Targets, except BMP4 achieved during the process of proteomic work in SOMALOGIC, INC.

Aptamers can be selected or designed by the many methods, including but not limited to as shown in Example 11. Non-limiting example of method of synthesis of aptamer is shown in Example 17. After aptamers are designed or selected to be used as binding elements on the device or stationary phase for Target extraction they should be linked to device or stationary phase. It could be done in many ways, including but not limited to as shown in Example 21.

In some embodiments, the disclosed protein binding device comprises binding element which is a polymer. A polymer is a large molecule, or macromolecule, composed of many repeated subunits. Polymer classes that is usable for binding element production include: Biopolymer, Inorganic polymer, Organic polymer, Conductive polymer, Copolymer, Fluoropolymer, Gutta-percha (Polyterpene), Phenolic resin, Polyanhydrides, Polyketone, Polyester, Polyolefin, (Polyalkene), Rubber, Silicone, Silicone rubber, Superabsorbent polymer, Synthetic rubber, Vinyl polymer and others.

Polymers are obtainable in many ways known in the art, including but not limited to polymerization, which is the process of combining many small molecules known as monomers into a covalently bonded chain or network. During the polymerization process, some chemical groups may be lost from each monomer. Laboratory synthetic methods are generally divided into two categories, step-growth polymerization and chain-growth polymerization.[e.g. As shown in Sperling, L. H. (Leslie Howard) (2006). Introduction to physical polymer science. Hoboken, N.J.: Wiley. ISBN 0-471-70606-X.] The essential difference between the two is that in chain growth polymerization, monomers are added to the chain one at a time only, such as in polyethylene, whereas in step-growth polymerization chains of monomers may combine with one another directly, such as in polyester. However, some newer methods such as plasma polymerization do not fit neatly into either category. Synthetic polymerization reactions may be carried out with or without a catalyst.

There are at least three main classes of biopolymers: polysaccharides, polypeptides, and polynucleotides. In living cells, they may be synthesized by enzyme-mediated processes, such as the formation of DNA catalyzed by DNA polymerase. The synthesis of proteins involves multiple enzyme-mediated processes to transcribe genetic information from the DNA to RNA and subsequently translate that information to synthesize the specified protein from amino acids. The protein may be modified further following translation in order to provide appropriate structure and functioning. There are other biopolymers such as rubber, suberin, melanin and lignin.

Different types of polymers and methods of its obtainment as well as the references to the more specific literature could be obtained in many sources, including but not limited to Wikipedia page https://en.wikipedia.org/wiki/Polymer, Painter, Paul C.; Coleman, Michael M. (1997). Fundamentals of polymer science: an introductory text. Lancaster, Pa.: Technomic Pub. Co. p. 1. ISBN 1-56676-559-5, McCrum, N. G.; Buckley, C. P.; Bucknall, C. B. (1997). Principles of polymer engineering. Oxford; New York: Oxford University Press. p. 1. ISBN 0-19-856526-7, Cowie, J. M. G. (John McKenzie Grant) (1991). Polymers: chemistry and physics of modern materials. Glasgow: Blackie. ISBN 0-412-03121-36, Ezrin, Myer. (1996). Plastics failure guide: cause and prevention. Munich; New York: Hanser Publishers: Cincinnati. ISBN 1-56990-184-8, Hall, Christopher (1989). Polymer materials (2nd ed.). London; New York: Macmillan. ISBN 0-333-46379-X, Lewis, P. R. (Peter Rhys); Reynolds, Ken.; Gagg, Colin. (2004). Forensic materials engineering: case study. Boca Raton: CRC Press. ISBN 0-8493-1182-9, Lewis, Peter Rhys (2010). Forensic polymer engineering: why polymer products fail in service. Cambridge [etc.]: Woodhead Publishing. ISBN 1-84569-185-7, Rudin, Alfred (1982). The elements of polymer science and engineering. Academic Press. ISBN 0-12-601680-1, Workman, Jerome; Workman, Jerry (2001). Handbook of organic compounds: NIR, IR, Raman, and UV-Vis spectra featuring polymers and surfactants. San Diego: Academic Press. ISBN 978-O-12-763560-6, Wright, David C. (2001). Environmental Stress Cracking of Plastics. RAPRA. ISBN 978-1-85957-064-7, Allcock, Harry R.; Lampe, Frederick W.; Mark, James E. (2003). Contemporary Polymer Chemistry (3 ed.). Pearson Education. p. 21. ISBN 0-13-065056-0.

In some embodiments this invention is a device described in this disclosure, wherein binding element is a peptide or therapeutic agent deleting, reducing, binding, deactivating, inhibiting or modulating or degrading at least one of the Targets in blood stream of subject or at least one effector upstream or downstream of at least one of the Targets is a peptide.

Peptides are short chains of amino acid monomers linked by peptide (amide) bonds. The covalent chemical bonds can be formed when the carboxyl group of one amino acid reacts with the amino group of another. The shortest peptides are dipeptides, consisting of 2 amino acids joined by a single peptide bond, followed by tripeptides, tetrapeptides, etc. A polypeptide is a long, continuous, and unbranched peptide chain. Peptides are divided into several classes, depending on how they are produced: Milk peptides Two naturally occurring milk peptides are formed from the milk protein casein when digestive enzymes break this down; they can also arise from the proteinases formed by lactobacilli during the fermentation of milk. Ribosomal peptides Ribosomal peptides are synthesized by translation of mRNA. They are often subjected to proteolysis to generate the mature form. Some organisms produce peptides as antibiotics, such as microcins. Since they are translated, the amino acid residues involved are restricted to those utilized by the ribosome. Nonribosomal peptides Nonribosomal peptides are assembled by enzymes that are specific to each peptide, rather than by the ribosome. The most common non-ribosomal peptide is glutathione. Other nonribosomal peptides are most common in unicellular organisms, plants, and fungi and are synthesized by modular enzyme complexes called nonribosomal peptide synthetases. These complexes are often laid out in a similar fashion, and they can contain many different modules to perform a diverse set of chemical manipulations on the developing product. These peptides are often cyclic and can have highly complex cyclic structures, although linear nonribosomal peptides are also common The different further details, classification of peptides and the methods to obtain peptides are widely known in the art, non-limiting list of the sources for additional details on it is given below: IUPAC. Compendium of Chemical Terminology, 2nd ed. (the “Gold Book”). Compiled by A. D. McNaught and A. Wilkinson. Blackwell Scientific Publications, Oxford (1997). XML on-line corrected version: http://goldbook.iupac.org (2006-) created by M. Nic, J. Jirat, B. Kosata; updates compiled by A. Jenkins. ISBN 0-9678550-9-8. doi:10.1351/goldbook.P04898, “What are peptides”. Zealand Pharma A/S. Archived from the original on 2014-07-14, Ardejani, Maziar S.; Orner, Brendan P. (2013-05-03), “Obey the Peptide Assembly Rules”. Science. 340 (6132): 561-562. Bibcode:2013Sci . . . 340 . . . 561A. doi:10.1126/science.1237708. ISSN 0036-8075. PMID 23641105.

The peptide binding elements can be easily fund by the methods known in the art, including but not limited to as shown in Example 10 and obtained through the methods known in the art, including but not limited to Example 16. After peptide Target binding element is identified to be used in device or on stationary phase it should be linked to it, that can be done in many ways, including but not limited to as shown in Example 20 or Example 23. Some non-limiting examples of peptide binding elements are shown in Example 22.

In some embodiments, the disclosed protein binding device comprises binding element which is a a small molecule. Given modern level of the development of small molecule binding elements identification and design technologies in most cases there are many ways to easily generate small molecule binding element for every Target, including but not limited to some of the methods described in Examples 12, 13 and 14. If intended to be used linked to the stationary phase, once developed or identified the small molecule binding element of Target should be linked to the stationary phase, that can be done in many ways known in the art, including but not limited to those described in Example 19, wherein also examples of method of synthesis of small molecules can be found. Some other examples of method of synthesis of small molecules can be found in Example 18. Some of the examples for small molecule Target binding elements are shown in this disclosure.

In some embodiments, this invention is a protein binding device, wherein the protein binding comprises binding elements, the binding elements bind or optionally—selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB and wherein device is configured to let fluid advance or pass to make it contact binding elements and optionally further configured to allow fluid to leave device after the contacting surface with binding elements, optionally further configured to let another portion of fluid to fill the space that the fluid that left the device vacated. In some embodiments, this invention is a described device having one port configured to let fluid in and out after contacting the fluid with binding elements. In some embodiments, this invention is a described device, further comprising at least one or any combination of features described for other devices in this disclosure. Such configurations allows to use the device for example in apheresis system, comprising device of this invention, to conduct extracorporeal online blood purification in bloodstream of the patient connected to such system, including possibility to proceed with the purification of large volumes of blood (substantially larger than the volume of blood contacting all surface of device with binding elements in one time).

In some embodiments, this invention is a protein binding device, wherein the protein binding device is at least one of the following or any combination of the following

a) comprises a housing, wherein the housing defines a lumen and the housing further comprises an inlet port for receiving fluid to the lumen, and an outlet port for releasing fluid from the lumen, wherein the lumen comprises binding elements,
b) comprises a housing of volume of more than 1 ml or more than 5 ml or more than 10 ml or more than 50 ml or more than 100 ml or more than 200 ml and binding elements,
c) comprises a housing, wherein the housing defines a lumen, a lumen comprising binding elements in the amount of more than 5 pmol, more than 10 pmol, more than 20 pmol, more than 50 pmol, more than 100 pmol, more than 0.3 μM, more than 0.5 μM, more than 1 μM, more than 5 μM or more than 10 μM, wherein the binding elements bind or optionally—selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In some embodiments the protein binding device disclosed herein comprises sorbent. In many cases binding elements are linked to sorbent, in some embodiments such sorbent fills the housing of device or column or other container through which the plasma is streamed. Sorbent can consist of variety of materials, including but not limited to polymers, silica gels, cotton, activated carbon, silica gel, resin, Amberlite resin, synthetic zeolite, natural zeolites, natural natrolite zeolite, modified kaolinite, carbon nanotubes or clays. Non-limiting list of possible sorbents: Polyvinylalcohol, Silica, Sepharose, Polyacrylate, hypercrosslinked polystyrene resin, Ethylene vinyl alcohol copolymer, Non-woven fabric made of polyethylene terephthalate, Polysulfone, Styrene divinylbenzene copolymer, Phenylalanine immobilized polyvinylalcohol gel, Tryptophan immobilized polyvinylalcohol gel, Polyethylene (coated by ethylene-vinylalcohol copolymer), Polyethylene, polystyrene divinylbenzene copolymer, polyacrylic acid, polymethacrylate, sepharose, dextran-sulfate cellulose, cellulose acetate, polyacrylate, polyester, sepharose, agarose, silica, and other many other polymer matrix or many other materials.

In some embodiments, this invention is a system, comprising protein binding device, comprising binding element to at least one of the Targets and configured to advance the fluid through the protein binding device, optionally by electromagnetic field or electromagnetic gradient to advance the fluid through the filter element, by pump etc.

In some embodiments, a protein binding device of this disclosure is a sorption device, comprising binding element to at least one of the Targets. The Targets can also be reduced extracorporeally or captured intracorporeally by sorption or other device.

In some embodiments binding elements to specific Target are linked to sorbent and sorbent is placed inside the container or column so the binding element is linked to the device in this way or in any other way. In some embodiments, binding elements can be linked to sorption device itself or in some embodiments to sorbent contained in it or both in many other ways. In some embodiment, the protein binding device and other device disclosed is sterile.

The particular implementations of this invention as device could be absolutely new device or in some embodiments it could be modifications of sorption or apheresis devices/columns known in the art, such as but not limited to Selesorb@, IM-TR 350@, IM-PH 350@, Prosorba®, Immunosorba@, Ig-Therasorb@, Miro, Immunoadsorption column LDL Lipopak, Lp(a) Lipopak®, Ig Adsopak®, ABO Adsopak®, Toxipak®, Hemopurifier, Plasmaflo, Cascadeflo®, Plasorba, Rheofilter®, Cureflo®, Cellsorba, H.E.L.P.™ apheresis, CytoSorb@, Seraph@, GLOBAFFIN®, IgEnio, Glycosorb ABO, LIXELLE, Adacolumn@ and many others known in the art (further in this application referred as Known Apheresis devices), wherein instead of binding elements existing in such columns or systems or in addition to existing construction of such devices the binding elements to Targets or Target capture agents or materials are linked or added wherein all other features of any particular Known Apheresis device which is being used as prototype are generally preserved, including but not limited to the manufacturing procedure, composition etc.

One of the many possible protein binding devices of this invention is described in Example 7. The protein binding device can be manufactured in many ways known in the art. Some of the possible ways to manufacture such devices can be easily adopted from the manufacturing of the known devices, including but not limited to mentioned in this disclosure. Another one non-limiting example is provided in Example 7.

It is known in the art that once one knows the binding element to Target it is trivial to generate or obtain its analogs having the same or weaker or stronger binding to Target which are also within the scope of this invention together with the protein binding device or medication or kit comprising such binding elements and corresponding methods. For example, some of the embodiments of this invention, e.g. protein binding device or medication or pharmaceutical composition and related methods comprises an isolated or produced Target binding antibody comprising a variable region heavy chain or light chain having at least 70%, 75%, 85%, 90%, 95%, 98% or 99% sequence identity with any of light or heavy chain of at least one antibody described as binding element in this application or any other protein or molecule mentioned in this disclosure or optionally having at least 75%, 70%, 85%, 90%, 95% or 98% sequence identity with any of light or heavy chain of at least one antibody described as binding element in this application, wherein such chain is binding to the Target or at least 75%, 70%, 85%, 90%, 95% or 98% sequence identity with the region binding the Target.

Some embodiments of this invention, e.g. protein binding device or medication or pharmaceutical composition and related methods comprise molecules having the same parts structurally or functionally identical or similar with the parts binding to the Target of the molecules described in this disclosure, or having the similar SAR characteristics.

In some embodiments this invention is an apheresis or plasma sorption system plasma filtering system or medical device or system or kit for reducing protein in fluid or device of this disclosure any element of it, comprising at least two blood plasma fractions, wherein concentration in second blood plasma fraction of protein selected from the Targets is less for at least 20%, at least 40%, at least 60%, at least 80%, at least 90%, at least 95%, at least 99%, at least 99.99% less in comparison with the level of such protein in first blood plasma fraction, optionally wherein plasma fraction is of a volume of at least 10 ml, at least 20 ml, at least 50 ml, at least 100 ml, at least 200 ml, at least 300 ml, optionally wherein the median difference in concentration of top 10, top 20, top 30 or top 50 elements indicated in ‘Biomarker” table in first and a second fraction is less than 5%, 10%, 20%, 30%, or 50%.

In a preferred embodiment a device described of this invention has a fluid volume capacity from 50 ml to 500 ml. Depending on different factors, the fluid volume capacity can be less or greater.

In some embodiments, the present disclosure is a device, apparatus or system for reducing at least one Target from the fluid and, optionally configured to produce a plasma product or pharmaceutical composition of this disclosure, comprising plasma fraction.

In a preferred embodiment, the device described in this disclosure is a column or apheresis column.

In some embodiments, the present disclosure is a protein binding device or pharmaceutical composition by which at least one method of this disclosure can be implemented.

In a preferred embodiment, device and treatment reduces Target or different Targets in fluid at a maximum scale, preferably leaving no and around zero Target in patient' fluid. At the same time, even minor reduction of Target can have a therapeutic effect. Thus, any reduction of Target or different Targets in fluid are useful, the more Targets are reduced the greater is therapeutic effect. Some non-limiting examples of modified plasma/pharmaceutical composition, comprising plasma fraction are shown in Example 29.

In a preferred embodiment, composition, comprising binding element of this invention binds, inhibits or degrades Target or different Targets in fluid at a maximum scale, preferably leaving no and around zero Target in patient' fluid uninhibited. At the same time, even minor binding, inhibition or degradation of Target can have a therapeutic effect. Thus, any binding, inhibition or degradation of Target or Targets in fluid are useful, the more Targets are bound, inhibited or degraded the greater is therapeutic effect.

The efficacy of devices, compositions, uses, binding elements, methods and other inventions described in this application can be shown in many ways, including but not limited to Example 26, Example 28, while one of many the possible ways to apply device on humans is shown in Example 27.

In some embodiments, a “pharmaceutical composition comprising a blood plasma fraction” means one selected of the following: a modified blood, modified serum or modified plasma or product made of anything of it.

In some embodiment, this invention is a plasma product, comprising plasma fraction, wherein blood plasma fraction comprises a negligible amount of a protein selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In some embodiment, this invention, is a plasma product, comprising plasma fraction of aged blood, wherein blood plasma fraction comprises a negligible amount of a protein selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In some embodiment, this invention is a plasma product, comprising plasma fraction of blood of human subject of 40 years old or elder, wherein blood plasma fraction comprises a negligible amount of a protein selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

In some embodiments, this disclosure provides a pharmaceutical composition comprising a blood plasma fraction, wherein the blood plasma fraction comprises a negligible amount of a protein selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB and wherein such composition comprises one or two or more biomarkers characteristic of aged subject.

In some embodiments, the one or two or more biomarkers (optionally—with associated measurement units in plasma) is a biological age characteristic or chronological age characteristic any of which is determined with the use of data from blood, including but not limited to data from blood serum, plasma, blood cells, whole blood etc. In some embodiments, Biological age or chronological age determined with the use of data from blood characterizes the health status or biological age or chronological age of the subject from whose blood the pharmaceutical composition comprising a blood plasma fraction is prepared. In some embodiments, the blood based biological age determination approach is described in prior art, including but not limited to any of the following publications, https://www.ncbi.nlm.nih.gov/pmc/articles/PMC5514388/. https://www.ncbi.nlm.nih.gov/pmc/articles/PMC4931851/, https://www.ncbi.nlm.nih.gov/pmc/articles/PMC5514388/ and corresponding references related to blood based biological age determination.

In some embodiments, the biological age is understood as the distance measured along a continuous trajectory consisting of distinct phases, each corresponding to subsequent human life stages as described in more details in “Quantitative Characterization of Biological Age and Frailty Based on Locomotor Activity Records”, Pyrkov et al., 2017) https://www.biorxiv.org/content/biorxiv/early/2017/09/09/186569.full.pdf

In some embodiments, the biological age is understood in the following context. The confinement of the aging dynamics of the physiological variables to the low-dimensional manifold representing the aging trajectory is a hallmark of criticality. It has been long suggested that the regulatory systems governing the dynamics of the organism state vector operate near the order-disorder boundary. The biological age is then the order parameter, associated with the organism development and aging, satisfies a stochastic Langevin equation in an unstable effective potential characterize by the single number, the underlying regulatory network stiffness. The number describes the organism state deviations from the youthful state and has the meaning of the number of regulatory abnormalities accumulated over the course of the organism life history, is associated with the decreased resilience and amplified risks of morbidities and death. We suggested that the stochastic biological age dynamics is the mechanistic origin of Gompertz mortality law. The exponential acceleration of the morbidity and mortality rates is the characteristic feature of aging in adult individuals or older. The reduction of the aging dynamics to essentially a one-dimensional manifold, a consequence of the criticality of the underlying regulatory network, means that the distance traveled along the aging trajectory is thus a progress indicator of the process of aging and hence is a natural biomarker of age. The biological age acceleration, i.e., the difference between the biological age of an individual and average the biological age prediction in the sex- and the age-matched cohort of their peers, is elevated for patients with chronic diseases. It is a powerful predictor of all-cause mortality even after confounding by the standard Health Risks Assessment (HRA) variables such as age, sex, and smoking status.

Some non-limiting examples of pharmaceutical compositions comprising binding elements, described in this disclosure are shown in Examples 31 and 32.

In some embodiments, the biological age is understood as the biomarker or metric based on one or more several biomarkers predicting risks of morbidity and/or death in 8 years or later or in range of mortality rate doubling time or later.

In some embodiments, this disclosure provides a pharmaceutical composition comprising a blood plasma fraction, wherein the blood plasma fraction comprises a negligible amount of a protein selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB and wherein such composition comprises one or two or more biomarkers characteristic of aged subject, who is understood as a subject with high mortality risk in about 1 month, in about 3 months, in about 6 months, in about 1 year, from about 1 month to about 6 months, from about 1 month to about 1 year, from about 1 year to about 3 years, from about 3 years to about 5 years, from about 5 years to about 8 years, from about 5 years to about 10 years, in about 5 years, in about 10 years, in about 15 years. In some embodiments, high mortality risk is a risk of dying from age related condition or disease. In some embodiments, high mortality risk is all cause mortality risk. Non limiting examples of blood based biomarkers of mortality and its critical volumes are described in prior art, including but not limited to https://www.ncbi.nlm.nih.gov/pmc/articles/PMC4899173/, https://www.ncbi.nlm.nih.gov/pmc/articles/PMC4454670/, https://www.ncbi.nlm.nih.gov/pmc/articles/PMC5334528/, Maximus Peto, Carlos De la Guardia, Ksenia Winslow, Andrew Ho, Kristen Fortney, & Eric Morgen. “Mortalitypredictors.org, a manually curated database of published biomarkers of human all-cause mortality. Aging, 2017.

In some embodiments, this disclosure provides a pharmaceutical composition comprising a blood plasma fraction, wherein the blood plasma fraction comprises a negligible amount of a protein selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB and wherein such composition comprises one or two or more biomarkers characteristic of subject with high morbidity risk in about 1 month, in about 3 months, in about 6 months, in about 1 year, from about 1 month to about 6 months, from about 1 month to about 1 year, from about 1 year to about 3 years, from about 3 years to about 5 years, from about 5 years to about 8 years, from about 5 years to about 10 years, in about 5 years, in about 10 years, in about 15 years. In some embodiments, high morbidity risk is a risk of acquiring an age related condition or disease.

In some embodiments, this disclosure provides a pharmaceutical composition comprising a blood plasma fraction, wherein the blood plasma fraction comprises a negligible amount of a protein selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB and wherein such composition comprises one or two or more biomarkers characteristic of subject with age related condition or disease or high risk of such disease, including but not limited to adult cancer, type 2 diabetes, age-related cardiovascular diseases, including but not limited to ischemic heart disease and stroke, metabolic syndrome, COPD, Alzheimer's disease etc., including but not limited those mentioned in this disclosure or at least one of the aging related declines. In some embodiments subject is understood as aged and having an aging related decline in case the corresponding parameter of subject health or appearance is changed into elder state in comparison with the own parameter of the same subject or in comparison with the median volume of the same parameter in statistically meaningful number of people of same gender of 25 years old in HNAHES study or statistically meaningful number of random people of same gender of 25 years old, optionally same race and residents of the same country or region.

In some embodiments, high (mortality or morbidity or age related disease or age related condition) risk is more than 90%, more than 80%, more than 70%, more than 60%, more than 50%, more than 40%, more than 30%, more than 20%, more than 10%, more than 5%, more than 3%, more than 1%, more than 0.5%, more than 0.1%, more than 0.05%.

In some embodiments, chronological age metric which gives estimation of chronological age of the subject from whose blood a pharmaceutical composition of this disclosure comprising a blood plasma fraction is prepared can be calculated by any applicable method known in the art or that will be developed in the future. In some embodiments, a chronological age metric is calculated as shown in example 9 “Chronological age calculation”.

In some embodiments if blood or plasma, which was taken from the subject to whom it is intended to be infused after the reduction if at least one of the Targets comprises characteristics of aged subject or at least one or two or more biomarkers characteristic of aged subject but after manipulations or interventions does not comprise such characteristic or biomarkers any more, a pharmaceutical composition produced from such blood having a negligible amount of at least one of the Targets is also encompassed by this disclosure.

In some embodiments this invention is a pharmaceutical composition of this disclosure or pharmaceutical composition comprising a blood plasma fraction, comprising a negligible amount of at least one of the Targets and is prepared from the blood of aged subject. In some embodiments, pharmaceutical composition of this disclosure comprises a blood plasma fraction of aged subject and a negligible amount of at least one of the Targets.

In some embodiments pharmaceutical composition of this disclosure comprising a blood plasma fraction, is obtained intracorporeally by deleting, reducing, binding, deactivating, inhibiting or degrading at least one of the Targets in blood stream of subject or by modulation (by deleting, reducing, binding, deactivating, inhibiting or degrading or by activating) at least one other effector upstream or downstream of at least one of the Targets, optionally wherein such modulation has an anti-aging effect. Some non-limiting examples of compositions prepared intracorporeally for the cases when at least one of the Targets are deleted, degraded, or its concentration in bloodstream of subject is reduced by any other way, e.g. by inhibiting some upstream target or mechanism, are suggested in Example 29 (the same examples as for examples of compositions after the use of protein binding device of this disclosure).

Some non-limiting examples of compositions prepared intracorporeally for the cases when at least one of the Targets are bound, deactivated or inhibited are suggested in Example 33. Such compositions can be produced by many ways, including but not limited to inhibition or binding at least one of the Target by some mean, including but not limited to antibody, peptide, small molecule, aptamer, nanoparticle, any other intervention, medication, other agent or force or intervention or medical device etc. In some embodiments such composition can be used for treatment, including but not limited to anti-aging treatment of the same subject in whose bloodstream such composition is obtained, and in some other embodiments such composition can be transfused to some other subject.

In some embodiments this invention is a pharmaceutical composition comprising a blood plasma fraction, wherein the blood plasma fraction comprises a complex of a protein and a binding element of such protein, wherein a protein is selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB and the binding element of such protein is selected from peptide, small molecule, antibody, aptamer, protein, virus, polymer, nanoparticle or particle. In some embodiments such binding element is artificial. In some embodiments such binding element is an therapeutic agent. In some embodiments such pharmaceutical composition comprises a negligible amount of protein remaining unbound by the binding element. In some embodiments, the protein in such pharmaceutical composition is bound by the binding element selected from the binding elements described in this application or its structural or functional analogs or by agent comprising the part binding such protein at least 99% structurally similar, at least 95% structurally similar, at least 90% structurally similar, or at least 80% structurally similar, or at least 70% structurally similar to the part binding such protein of at least one of the binding elements described in this application.

In some embodiments this invention is a pharmaceutical composition comprising a blood plasma fraction, wherein at least one of the proteins selected from the Targets is deleted, reduced, bound, inhibited or degraded to Negligible amount, while at least 20%, 30%, 40%, 50%, 60%, 70%, 80% of components of such plasma shown in Table “Biomarkers” are in “40+” or “60+” state or the median difference of concentration in percentage of such parameters from “40+” or “60+” state is less than 50%, 40%, 30%, 20%, 10%, 5%, 1%.

In some embodiments this invention is pharmaceutical composition, comprising a blood plasma fraction, and wherein such composition comprises one, two or more biomarkers characteristic of aged subject and wherein at least one of Targets is deleted, reduced, deactivated, bound, inhibited or degraded to the level that at least one function of such Target or all same proteins is reduced by at least 20%, at least 30%, at least 40%, at least 50%, at least 60%, at least 70%, at least 80%, at least 90%, at least 95%, at least 99%, at least 99.9%, at least 99.99%.

In some embodiments this invention is a pharmaceutical composition comprising a blood plasma fraction, wherein the blood plasma fraction comprises one, two or more biomarkers characteristic of aged blood, and wherein the blood plasma fraction comprises a negligible amount of a protein selected from Targets, while the median difference of concentration in percentage of components which are the top 10, top 20 or top 50 or all biomarkers from “Biomarkers” table in such composition in comparison with the same metric in plasma of subject from whose blood the composition is made or in comparison with the same metric in plasma of people of same health status, chronological age or biological age, is substantially less or at least 2 fold less than the level of Target concentration reduction from its original level in plasma of the subject from whose blood th composition is prepared or from the median level of the Target in plasma of people of same health status, chronological age or biological age.

In some embodiments this invention is a pharmaceutical composition comprising a blood plasma fraction, wherein concentration of at least one protein selected from the Targets is reduced at least to 20%, 40%, 60%, 80%, 90%, 95%, 99%, 99.99% in comparison with the level of such protein in original plasma from which such plasma product is made.

In some embodiments this invention is a pharmaceutical composition comprising a blood plasma fraction, wherein concentration of at least one protein selected from the Targets is selectively reduced at least to 20%, 40%, 60%, 80%, 90%, 95%, 99%, 99.99% in comparison with the level of such protein in original plasma from which such plasma product is made, or optionally further the median difference in concentration of top 10, top 20, top 30 or top 50 elements indicated in ‘Biomarker” table in pharmaceutical composition and original plasma is less than 5%, 10%, 20%, 30%, or 50%.

In some embodiments this invention is a pharmaceutical composition comprising a blood plasma fraction, wherein the blood plasma fraction comprises a negligible amount of a protein selected from Targets and wherein the median difference in concentrations in percentage of all or of top 10 or of top 20 components shown in “Biomarkers” table from the concentration of such components in original plasma from which such composition is made is substantially less than the level to which the Target was reduced or at least 50% less or, wherein Target is selectively reduced in comparison with level of reduction of at least 30% of components shown in “Biomarkers” table.

In some embodiments this invention is a pharmaceutical composition comprising a blood plasma fraction, wherein the blood plasma fraction comprises one, two or more biomarkers characteristic of aged blood, and wherein it is produced by deactivation, inhibiting, deleting, degrading or binding at least one of the Targets to the level having biological or anti-aging effect equal or comparable to the effect of reducing such Target to negligible amount, including but not limited to by the devices, pharmaceutical compositions and methods disclosed herein.

In some embodiments this invention is a pharmaceutical composition of this disclosure for anti-aging treatment. In some embodiments such pharmaceutical composition infused into the patient or administered in any other way or prepared in bloodstream of the patient in therapeutically effective amount and regimen.

In some embodiments such composition can be obtained at least one of the many ways, including but not limited to ex-vivo (for example, but not limited to—by sorption or filtering out at least one of the Targets from natural plasma etc) or intracorporeally in human (in patient or donor) or animal organism by administering or generating inside body agents binding Targets or degrading or causing Target binding or degradation or Target inactivation.

In some embodiments, this invention is a pharmaceutical composition produced by the device of this invention or by agent or by method described in this invention.

In some embodiment, any one or any combination of the Targets can be removed from the blood by the means of extracorporeal sorption or filtering device or any other mean to remove at least one of Targets, while the rest of the plasma or the most of the rest of the plasma can be returned back into the patient so the pharmaceutical composition of this invention comprising plasma fraction can be obtained in many ways, including but not limited to by streaming blood plasma through the sorption column/device described in this application.

In some embodiments such composition comprising plasma differs from the natural plasma of patient or plasma donor by the fact, that at least one of the Targets or any combination of Targets are inhibited, bound, removed to the negligible amount, degraded, deleted to the negligible amount or deactivated in any way while the concentration of the most other of elements of such plasma are on its “natural” level or close to it, because in case of “natural” plasma all or most elements of such plasma are in the state corresponding to the relative chronological age, biological age and health status of the person from whose plasma the pharmaceutical composition of this inventions is obtained. In some embodiments of this invention the pharmaceutical composition comprising plasma fraction will have all (or most of the plasma circulating proteins) in the concentrations/amounts corresponding to the certain relative age, biological age or health status, while one or several proteins selected from the Targets will be present in zero or reduced amounts (Negligible amount) or the amount of the active Targets will be less than in “natural” plasma from which the plasma product is manufactured.

There are many ways to technically describe plasma and distinguish plasma taken from the aged subject from those taken from the younger subject and plasma with the at least one Target reduced or deactivated. There are alot of elements of blood plasma changing with the age and metrics based on it which can be indicative for the chronological or biological age and/or health status of the person from whose blood the pharmaceutical composition comprising plasma fraction had been made, such as proteins, metabolites etc and there are many methods to calculate a biological or chronological age of the person whose plasma is used is known and new methods of calculation are constantly being introduced. Any of such methods to calculate a biological or chronological age of the person whose plasma is used can be used to define plasma as aged or made from the aged blood or comprising biomarkers of aged blood.

One of the ways to estimate age of the plasma donor it is to describe what elements in what amount are contained in blood plasma and compare it to the data regarding known concentration/amounts of those of such elements in blood plasma which amount changes with the age, known in the art. As a non-limiting example, some of such elements of plasma are shown in the table “Biomarkers”. These biomarkers can be measured and analyzed by the methods known in the art, as an example the current “Biomarker” table contains parameters and their values found in blood plasma of people of different age in The National Health and Nutrition Examination Survey (NHANES) (a program of studies designed to assess the health and nutritional status of adults and children in the United States). In some embodiments ‘Old” or “Aged” level of proteins or other plasma elements means the level of plasma proteins or other elements of plasma which concentration is changing with the age or their combination or metric based on such plasma proteins or other elements that corresponds to the median or average level of the of people aged at least 30, 35, 40, 45, 50, 55, 60, 65, 70, 75, 80, 85, 90 and elder is known in the art and can be measured by many methods known in the art and yet to be introduced.

One of the many possible ways to use elements shown in “Biomarkers” table or others known in the art as changing with age for the evaluation of chronological age of the person is to use a decision tree as in Decision tree example 9. Many “aged” blood plasma examples are also available in Example 9.

There are many different ways and tools to measure expression amount of the particular proteins in blood plasma known in the art that can also be used for evaluation if the person whose blood is used is aged. The non-limiting examples of such technologies and tools are SOMAscan® Assay of Somalogic (http://somalogic.com), biomarker panels of Olink Proteomics (http://www.olink.com/), ELISA, multiplexes comprised of antibodies binding to the particular proteins e.g. Luminex technology (https://www.rndsystems.com/products/luminex-assays-and-high-performance-assays), mass spectrometry etc.

Though technologies to estimate the amounts of proteins are different and usually use the different units most of them can give the estimation on the relative amount of each protein in plasma and how this amount changes with the age, biological age and health status.

The pharmaceutical composition comprising plasma fraction of this invention can be immediately infused into the same patient from whose plasma it is obtained or in other patient or infused in any of them later in same or other location (for example after some delay or preservation) or generated inside the bloodstream of the patient or donor or used in any other reasonable way.

In some embodiments this invention is a method, including but not limited to method of testing of efficacy of protein binding device or any other device reducing protein, selected of the group of Targets, comprising the checking in the patient treated by such device (or to whom the plasma treated by such device was infused) at least one of the following: biological age of the patient, at least one aging biomarker, at least one age related deficit or disease, at least one of rejuvenation marker, frailty, health span or life span or in any other reasonable method to check the efficacy of anti-aging treatment. In many embodiments, in case any anti-aging effect of such device implementation is spotted the device is deemed to be effective.

In some embodiments this invention is a method, including but not limited to method of testing of efficacy of therapy deleting, reducing, binding, inhibiting or degrading at least one of the proteins, selected of the group of Targets or therapy modulating (by deleting, reducing, binding, deactivating, inhibiting or degrading or by activating or by any other way) at least one other effector upstream or downstream of at least one of the Targets, optionally wherein such modulation has an anti-aging effect, comprising the checking in the subject treated by such therapy at least one of the following: checking biological age of the patient, at least one aging biomarker, at least one age related deficit or disease, at least one of rejuvenation marker, frailty, health span or life span, or any other marker or parameter reasonable for checking in testing of anti-aging therapy efficacy, optionally wherein therapy is a monoclonal or polyclonal antibody, optionally humanized, which recognizes the receptor of at least one respective Target, protein, aptamer, peptide, polymer, virus or small molecule, binding or inhibiting or degrading a respective Target or any molecule or composition described in this disclosure or its analog.

In some embodiments checking of efficacy of device or therapy/measurement of markers or symptoms of related diseases or conditions is conducted in 1 month after the infusion of treated plasma or administration of therapy in therapeutically effective amount, in 3 months, in 6 months, in 12 months, in 18 months, in 24 months or in 36 months after such infusion, or in around such date, or in date reasonably defined by the doctor based on the parameter being measured and other factors known to the expert in the field.

In some embodiments this invention is a tangible medium comprising a computer program, which, when executed, causes a medium to perform a method comprising: attribution to the information about a subject an information about a treatment or therapy related to deactivating, deleting, reducing, binding, inhibiting or degrading at least one of the proteins, selected of the group of Targets, or in some embodiments to treatment or therapy related to modulating or binding or inhibiting or degrading or activating at least one effector upstream or downstream of at least one of the Targets, optionally, wherein such modulation or binding or inhibiting or degrading or activating has anti-aging effect, optionally wherein treatment is anti-aging treatment, optionally wherein such deactivating, deleting, reducing, binding, inhibiting or degrading is achieved by device or composition or agent described in this disclosure, optionally further comprising attributing to the information about patient before or after or before and after the treatment to information about checking of at least one selected from the group: biological age of the patient, at least one aging biomarker, at least one age related deficit or disease, at least one of rejuvenation marker, frailty, health span or life span.

An example of such tangible medium could be a APPLE™ 2014 MACBOOK AIR™ 13″ Intel™ i5 with Microsoft™ Excel™ installed and executed on it, wherein to patient with name John Junior Smith (born 2 Jan. 1937) the information about reducing of GDF15, FSTL3, BMP4 and FRZB is attributed in the sense that is logically linked as an information in Excel table (in this example attribution is realized as placing the information about treatment by reducing some of the Targets in the same line in the file with the name and ID of the patient to whom such treatment is prescribed) and allows easy finding of patients to whom such treatment is prescribed and other processing of such information given that Gero device reduces GDF15, FSTL3, BMP4 and FRZB in blood plasma in extracorporeal procedure.

| | | Date | | |
|---|---|---|---|---|
| patient ID | Name | of birth | diagnosis | Treatment prescribed |
| 28282839 | John | 2 Jan. | Before treatment: | Extracorporeal sorption |
| | Junior | 1937 | moderate frailty, | of GDF15, FSTL3, |
| | Smith | | moderate cognitive | BMP4 and FRZB |
| | | | decline, hand grip | proteins from plasma |
| | | | strength −21.3 kg | by Gero sorption |
| | | | and other age | column* |
| | | | related deficites | |

*Where Gero sorption column selectively reduces GDF15, FSTL3, BMP4 and FRZB in blood plasma in extracorporeal procedure

Processors suitable for the execution of a computer program related to this invention include, by way of example, both general and special purpose microprocessors, and any one or more processors of any kind of digital computer. Generally, a processor receives instructions and data from a read-only memory or a random access memory or both. The essential elements of a computer are a processor for executing instructions and one or more memory devices for storing instructions and data. Generally, a computer also includes, or be operatively coupled to receive data from or transfer data to, or both, one or more mass storage devices for storing data, e.g., magnetic, magneto-optical disks, or optical disks. Data transmission and instructions can also occur over a communications network. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

In some embodiments this invention is a tangible medium comprising a computer program, which, when executed, causes a device to perform a method comprising: attribution to the information regarding protein binding device or medical device or any other device or therapeutic agent or composition an information about deactivating, deleting, reducing, binding, inhibiting or degrading at least one of the proteins, selected of the group of Targets, or in some embodiments an information about modulating or binding or inhibiting or degrading or activating at least one effector upstream or downstream of at least one of the Targets, optionally, wherein such modulation or binding or inhibiting or degrading or activating has anti-aging effect or the purpose of such action is to induce anti-aging effect, optionally, wherein information about deleting, reducing, binding, inhibiting or degrading respective protein or other element is associated with the information about anti-aging treatment, optionally wherein device or agent is described in this disclosure.

In some embodiments this invention is a tangible medium comprising a computer program, which, when executed, causes a medium to perform a method comprising: attribution to the information about a therapy, agent, composition, device, medium or procedure associated with deletion, reduction, binding, inhibiting or degrading at least one of the proteins, selected from the group of Targets to the information related to treatment or to anti-aging treatment.

As an example of such attribution the excel file executed on the same computer as described above or website or webpage available in Internet hosted on the server e.g. www.ipage.com, can be suggested, any of which when executed show at list one line of the following:

| | Agent/Mechanism of | |
|---|---|---|
| Name | Action/Mode of action | Indication |
| ASD1 | anti-GDF15 antibody | anti-aging, anti-frailty, amelioration of |
| | | moderate cognitive decline, amelioration |
| | | hand grip strength lose and amelioration |
| | | of other age related deficits |
| GER23 | peptide inhibitor of | anti-aging, anti-frailty, amelioration of |
| | FRZB | moderate cognitive decline, amelioration |
| | | hand grip strength lose and amelioration |
| | | of other age related deficits |
| GER56 | *Gero sorption column | anti-aging, anti-frailty, amelioration of |
| | (selectively reduces | moderate cognitive decline, amelioration |
| | GDF15, FSTL3, BMP4 | hand grip strength lose and amelioration |
| | and FRZB in blood | of other age related deficits |
| | plasma) | |
| FDS89 | small molecule | anti-aging, anti-frailty, amelioration of |
| | inhibitor of BMP4 | moderate cognitive decline, amelioration |
| | | hand grip strength lose and amelioration |
| | | of other age related deficits |
| NP882 | Anti-GDF15 | anti-aging, anti-frailty, amelioration of |
| | Nanoparticle | moderate cognitive decline, amelioration |
| | | hand grip strength lose and amelioration |
| | | of other age related deficits |

In some embodiments this invention is a method, comprising an attribution to information about the patient an information about the treatment related to deactivating deleting, reducing, binding, inhibiting or degrading at least one of the proteins, selected of the group of Targets, information about the modulating or binding or inhibiting or degrading or activating at least one effector upstream or downstream of at least one of the Targets, optionally, wherein such modulation or binding or inhibiting or degrading or activating has anti-aging effect, wherein such attribution is performed in database or medium, comprising a computer program, which, when executed, causes a medium to perform such attribution or in other medium, optionally wherein the treatment is described as using a sorption or protein binding device or attributed to sorption or protein binding device or to blood plasma sorption procedure, including but not limited to at least one of devices or procedures described in this disclosure.

In some embodiments this invention is a method, comprising an attribution of information about the patient to an information about the agent deactivating, deleting, reducing, binding, inhibiting or degrading at least one of the proteins, selected of the group of Targets or about the agent modulating or binding or inhibiting or degrading or activating at least one at least one effector upstream or downstream of at least one of the Targets, optionally, wherein such modulation or binding or inhibiting or degrading or activating has anti-aging effect, optionally wherein agent is selected from the group: a monoclonal or polyclonal antibody, protein, aptamer, peptide, polymer, virus or small molecule, nanoparticle or any identification meaning such agent or composition, or to the information related to treatment associated with deletion, reduction, binding, inhibiting or degrading at least one of Targets, wherein such attribution is performed in database or medium, comprising a computer program, which, when executed, causes a medium to perform such attribution or in other medium, optionally wherein inhibiting or binding of respective Target is achieved by at least one of the agent selected from the binding elements described in this application or is its analog.

In some embodiments this invention is a method, comprising attribution of information about the therapy, agent, device, medium or procedure associated with deactivation, deletion, reduction, binding, inhibiting or degrading at least one of the proteins, selected from the group of Targets) to the information related to treatment, optionally—anti-aging treatment, wherein such attribution is performed in database or medium, comprising a computer program, which, when executed, causes a medium to perform such attribution or in other medium, optionally to the labeling information related to medication or medical device.

In some embodiments this invention is a method of this disclosure, comprising attribution of information where in the patient age is above 30 years old or above 40 years old or above 50 years old and/or the patient is someone who is in need of anti-aging treatment, optionally, wherein agent is selected from the group: a monoclonal or polyclonal antibody, optionally humanized, protein, aptamer, peptide, polymer, nanoparticle, virus or small molecule, or other agent described as binding element in this application, or its analog or information about pharmaceutical composition, comprising such agent or its analog or any ID/identification meaning such agent or composition or wherein device is a protein binding device or procedure is a blood plasma sorption procedure or optionally, wherein treatment is an anti-aging treatment.

In some embodiments this invention is a method, or a tangible medium comprising a computer program, which, when executed, causes a medium to perform a method comprising step of attributing to agent or device of this invention an information comprised in notice, description or instruction described in this disclosure for kits, comprising notice, description or instruction.

In some embodiments the method of this invention comprising attribution of information described in this disclosure is a computer implemented method. In some embodiments this invention is a method, the method of this invention, comprising attribution of information executed on the medium of this invention and described in corresponding part of this disclosure related to such medium.

In some embodiments this invention is a tangible medium or computer system or processor, comprising a executable instruction or computer program, which, when executed, causes a medium to perform a method comprising attribution of information described in this disclosure.

In some embodiments this invention is an apparatus to execute method described in this disclosure the apparatus comprising the processor comprising the tangible medium described in this disclosure.

One of the technical problems underlying the present invention was to identify alternative and/or improved means and methods of anti-aging treatment, wherein the reduction and other modulation of Targets may have beneficial effect and corresponding methods. The solution to this technical problem is achieved by providing the embodiments characterized in this application and items.

Animal Treatments/In Vivo Experiments

In order to evaluate the effect of circulating target inhibition on frailty and frailty-related phenotypes in C57BL/6 male mice aged 106 weeks were treated with FSTL3 blocking/neutralizing antibody (AF1255, https://www.rndsystems.com/products/mouse-follistatin-related-gene-protein-flrg-antibody_af1255) once via IV injection. The mice from another group were treated with 42 ppm of a well-characterized life-extending drug—rapamycin as the positive control. We choose to use rapamycin since its life-extending properties were confirmed in multiple experiments, including a large study within the NIH (NIA) Intervention Testing Program (https://www.nia.nih.gov/research/dab/interventions-testing-program-itp).

The effects of the interventions in the animals were evaluated with the help of the Frailty Index and Open Field Test. We also measured markers of senescence in peripheral lymphocytes, synaptoplasticity, and, ultimately, survival.

Animal Survival Rates and Lifespan

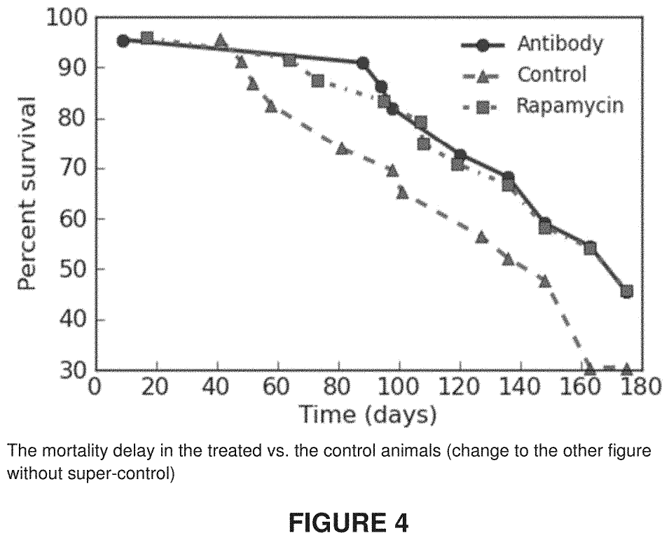

The survival curves from the experiment are shown in FIG. 4. Animals in the antibody- and in the rapamycin treatment groups demonstrated a significant improvement in lifespan (roughly a month of mortality delay, see FIG. 4).

Frailty Index

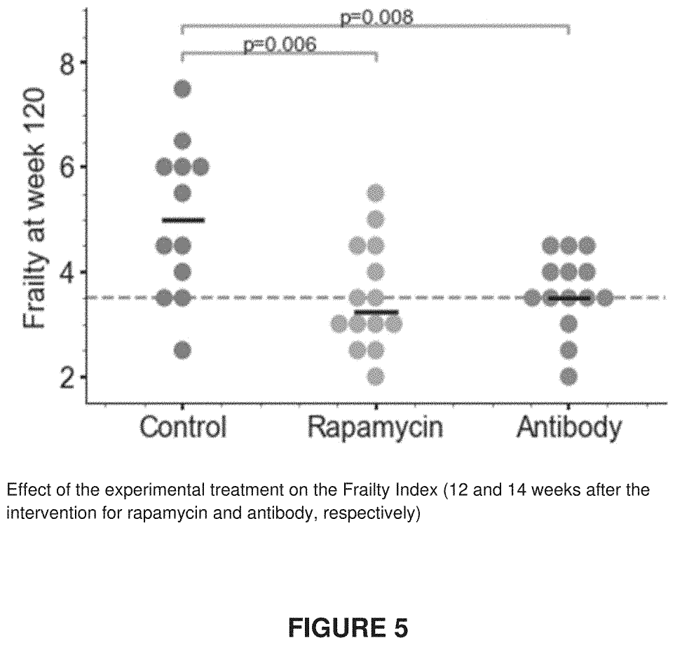

The Frailty Index (FI) was measured as in White et al. (Whitehead J C, Hildebrand B A, Sun M, Rockwood M R, Rose R A, Rockwood K, Howlett S E. A clinical frailty index in aging mice: comparisons with frailty index data in humans. J Gerontol A Biol Sci Med Sci. 2014; 69(6):621-32) and consists of 31 phenotypes that are indicators of age-associated health deterioration including alopecia, physical/musculoskeletal, auditory, ocular, nasal, signatures of digestive and other disorders. Each phenotype was scored on a 0, 0.5 or 1 scale, based on its severity. The 31 metrics share many characteristics of the human frailty indicators and were previously reported to progress similarly with aging in mice and humans (see Kane, A. E., et al., Animal models of frailty: current applications in clinical research. Clin Interv Aging, 2016; 11:1519-1529). As shown in FIG. 5, treatment with the target-blocking antibody as well as rapamycin demonstrated a significant reduction of Frailty Index.

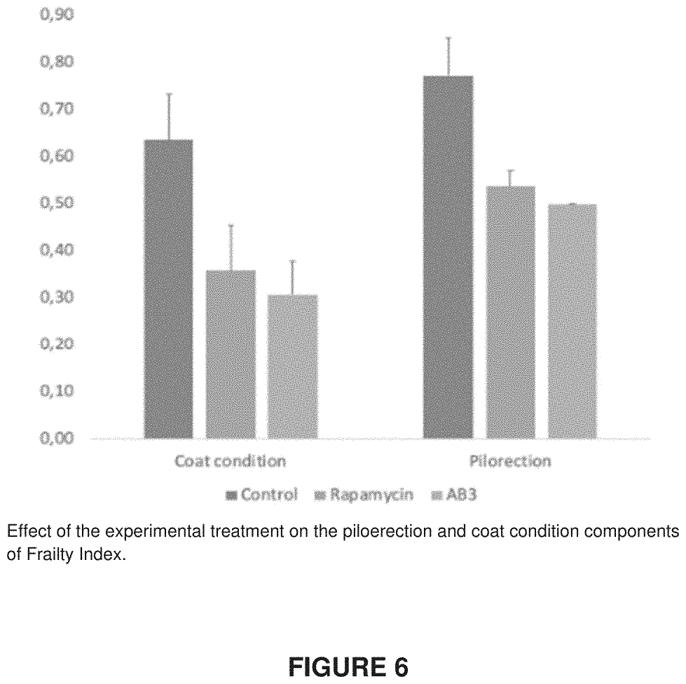

As shown in FIG. 6, antibody and rapamycin treatment significantly reduced the severity of piloerection and poor coat condition phenotypes.

Open Field Test

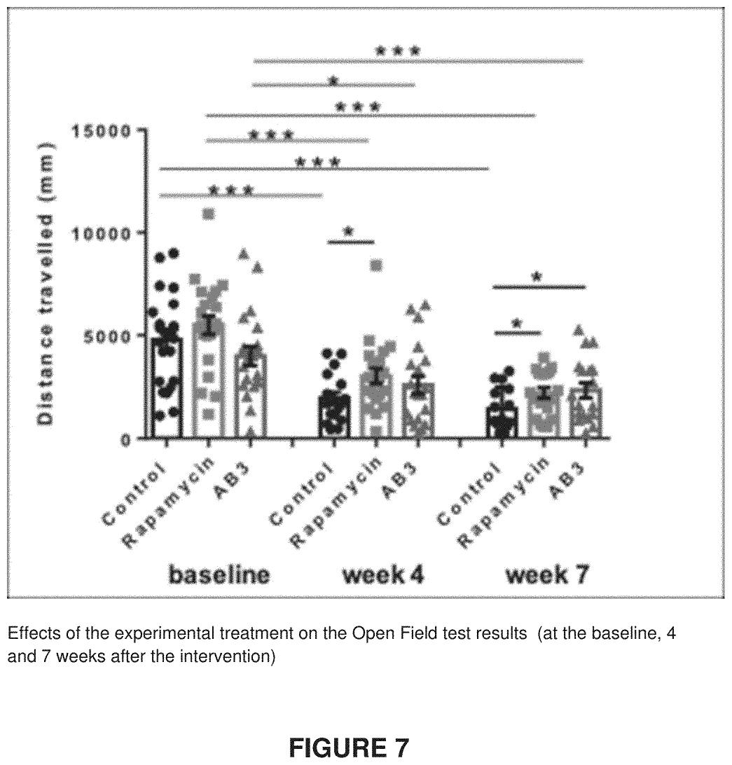

Locomotor activity recordings were carried out using a square open field. Locomotor activity data including total distance traveled (mm) and velocity (mm/s) were measured. Animal motor function deteriorated significantly while ageing. At 4 weeks and 7 weeks after the start of rapamycin treatment (baseline), mice showed significant improvement in motor function compared to the control group. At 5 weeks after ab treatment (7 weeks after baseline measurement), mice showed significant improvement in motor function compared to the control group (see FIG. 7).

Senescence

Aging is associated with an increasing burden of senescent cells (SCs) (see Burd C E, Sorrentino J A, Clark K S, Darr D B, Krishnamurthy J, Deal A M, Bardeesy N, Castrillon D H, Beach D H, Sharpless N E. Monitoring tumorigenesis and senescence in vivo with a p16(INK4a)-luciferase model. Cell. 2013; 152(1-2):340-51). Senesce is an irreversible growth cycle arrest that occurs due to accumulated DNA damage and other types of cellular stress, such as oncogene activation and telomere attrition. Besides these “natural” forms of stress, senescence may also be induced in response to chemotherapy. Chemotherapy-induced senescence, like apoptosis, was initially seen as a favorable outcome of chemotherapy. However, specific populations of cancer cells can escape genotoxic treatments and induce disease relapse (see Guillon J, Petit C, Toutain B, Guette C, Lelievre E, Coqueret O. Chemotherapy-induced senescence, an adaptive mechanism driving resistance and tumor heterogeneity Cell Cycle. 2019; 18(19):2385-2397). Moreover, most genotoxic treatments induce senescence in normal tissue, causing permanent damage to a wide range of organs and contributing to accelerated aging observed in cancer survivors.

SCs are characterized by an acquisition of a pro-inflammatory senescence-associated secretory phenotype (SASP) and often by an increased expression of the cyclin-dependent kinase inhibitor protein p161nk4a and beta-galactosidase. Expression of p161NK4a has been shown to markedly increase with aging in most mammalian tissues, including human, and caloric restriction, which retards aging in rodents, attenuates this age-induced increase in p161NK4a (see Krishnamurthy J, Torrice C, Ramsey M R, Kovalev G I, Al-Regaiey K, Su L, Sharpless N E Ink4a/Arf expression is a biomarker of aging. J Clin Invest. 2004; 114(9):1299-307).

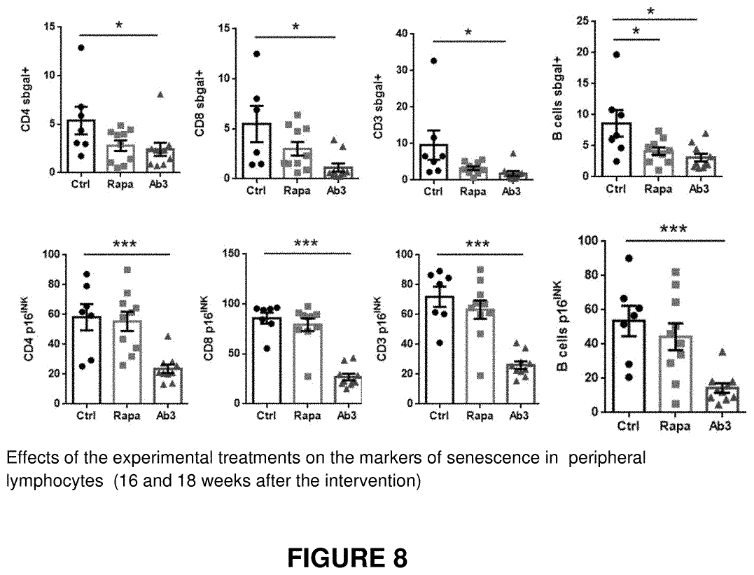

In a study using human peripheral lymphocytes (see Liu Y, Sanoff H K, Cho H, Burd C E, Torrice C, Ibrahim J G, Thomas N E, Sharpless N E. Expression of p16(INK4a) in peripheral blood T-cells is a biomarker of human aging. Aging Cell. 2009; 8(4):439-48), it was shown that the expression of p161NK4a correlated with donor chronologic age, smoking status, physical inactivity, and plasma interleukin-6 concentration, a marker of human frailty. These data suggest that p161NK4a expression in peripheral lymphocytes may be used as a blood biomarker of frailty. FIG. 8 shows that animals from the antibody treatment group show a significantly lower number of senescent cells in all lymphocyte subpopulations compared to control animals.

Synaptoplasticity

Progressive reduction of structural and functional plasticity is associated with the gradual decline in cognitive function. Moreover, chemotherapy-induced cognitive decline is associated with loss of synaptic integrity and it was demonstrated that cisplatin-treated mice show reduced levels of PSD95 and synaptophysin (see Chiang A C A, Huo X, Kavelaars A, Heijnen C J. Chemotherapy accelerates age-related development of tauopathy and results in loss of synaptic integrity and cognitive impairment. Brain Behav Immun. 2019; 79:319-325).

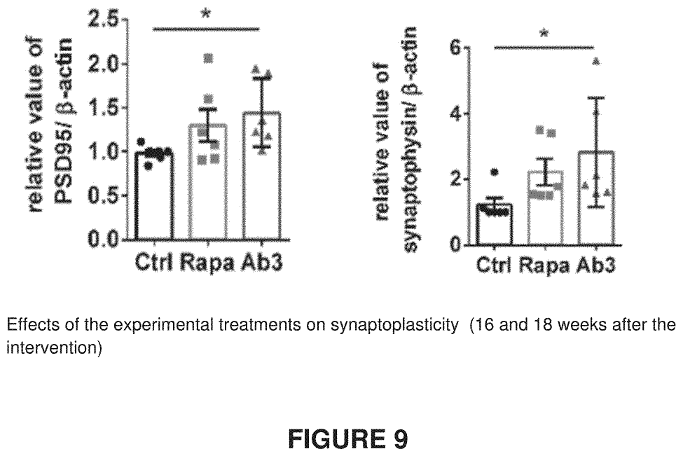

Synaptophysin immunoreactivity has been identified as a useful marker for presynaptic density, whereas PSD95 is a marker of post-synaptic density. In order to analyze synaptic plasticity in our experiment, Western blot analysis of mouse brain tissue was performed using antibodies against PSD95 and synaptophysin. As shown in FIG. 9, the expression of PSD95 and synaptophysin was significantly higher in the Antibody treatment group compared to the control group.

Trachea Stem Cells

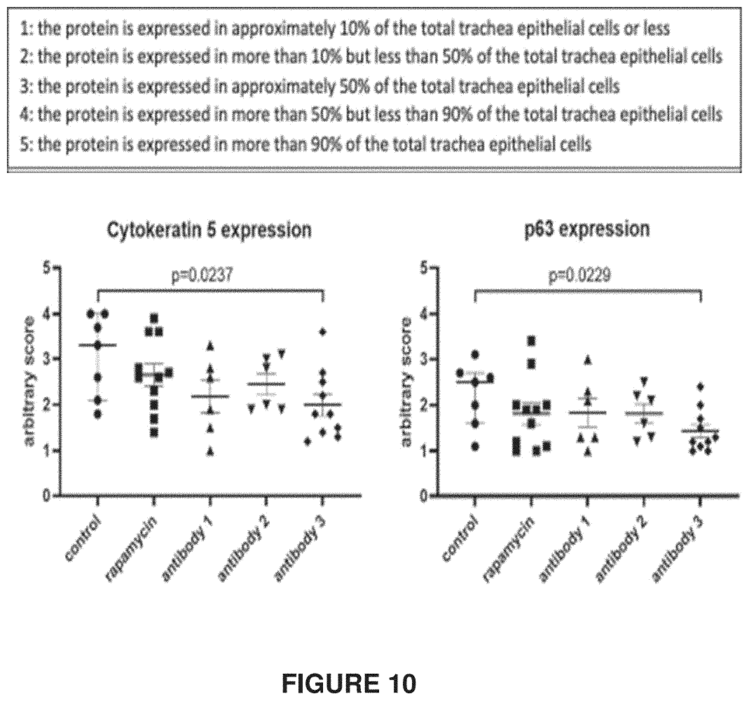

The staining was performed on paraffin-embedded trachea using antibodies against cytokeratin 5 (Krt5) and p63 (1 section per mouse). The protein expression in the whole trachea section was semi-quantified blindly by 1 person under 40× microscope, using an arbitrary score 1-5 as described and shown in FIG. 10. The smallest trachea section resulted in 5 counts and the largest trachea section resulted in 25 counts (the number of counts refers to the number of microscopic fields needed to cover the whole trachea section). The average score for each section was calculated.

Each dot in the graphs of FIG. 10 represents an average semi-quantitative score of each trachea section. The bars in FIG. 10 represent median±IQR. Mann-Whitney U test was performed to compare each treatment with the control group. Significant difference was found only between the antibody 3 and control.

### ADDITIONAL REFERENCES

Chen J, Lau Y F, Lamirande E W, Paddock C D, Bartlett J H, Zaki S R, Subbarao K. Cellular immune responses to severe acute respiratory syndrome coronavirus (SARS-CoV) infection in senescent BALB/c mice: CD4+ T cells are important in control of SARS-CoV infection. J Virol. 2010; 84:1289-301.
Interleukin-6 in COVID-19: A Systematic Review and Meta-Analysis https://www.medrxiv.orq/content/10.1101/2020.03.30.20048058v1
Anti-IL-6 Agents Suggested by SITC to Treat Patients with COVID-19 https://www.cancernetwork.com/news/anti-il-6-aqents-suqqested-sitc-treat-patients-covid-19

Accordingly, the present invention also relates to the following items.

Items
1. A protein binding device, wherein the protein binding device comprises a housing, wherein the housing defines a lumen and the housing further comprises an inlet port for receiving fluid to the lumen, and an outlet port for releasing fluid from the lumen, wherein the lumen comprises binding elements, wherein the binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
2. The protein binding device of item 1, wherein the binding elements selectively bind the protein CCDC80.
3. The protein binding device of item 1, wherein the binding elements selectively bind the protein CD59. The protein binding device of item 1, wherein the binding elements selectively bind the protein CD59.
4. The protein binding device of item 1, wherein the binding elements selectively bind the protein CHRDL1.
5. The protein binding device of item 1, wherein the binding elements selectively bind the protein COL18A1.
6. The protein binding device of item 1, wherein the binding elements selectively bind the protein CST3.
7. The protein binding device of item 1, wherein the binding elements selectively bind the protein DPT.
8. The protein binding device of item 1, wherein the binding elements selectively bind the protein EFEMP1.
9. The protein binding device of item 1, wherein the binding elements selectively bind the protein FAS.
10. The protein binding device of item 1, wherein the binding elements selectively bind the protein FSTL3.
11. The protein binding device of item 1, wherein the binding elements selectively bind the protein GAS1.
12. The protein binding device of item 1, wherein the binding elements selectively bind the protein GDF15.
13. The protein binding device of item 1, wherein the binding elements selectively bind the protein KLK11.
14. The protein binding device of item 1, wherein the binding elements selectively bind the protein MMP7.
15. The protein binding device of item 1, wherein the binding elements selectively bind the protein NBL1.
16. The protein binding device of item 1, wherein the binding elements selectively bind the protein NTN1.
17. The protein binding device of item 1, wherein the binding elements selectively bind the protein POSTN.
18. The protein binding device of item 1, wherein the binding elements selectively bind the protein PTN.
19. The protein binding device of item 1, wherein the binding elements selectively bind the protein RELT.
20. The protein binding device of item 1, wherein the binding elements selectively bind the protein SFRP1.
21. The protein binding device of item 1, wherein the binding elements selectively bind the protein SMOC1.
22. The protein binding device of item 1, wherein the binding elements selectively bind the protein STC1.
23. The protein binding device of item 1, wherein the binding elements selectively bind the protein TNFRSF1A.
24. The protein binding device of item 1, wherein the binding elements selectively bind the protein UNC5C.
25. The protein binding device of item 1, wherein the binding elements selectively bind the protein sFRP-3.
26. The protein binding device of item 1, wherein the binding elements selectively bind the protein TNFRSF1B.
27. The protein binding device of item 1, wherein the binding elements selectively bind the protein CD55.
28. The protein binding device of item 1, wherein the binding elements selectively bind the protein BMP4.
29. The protein binding device of item 1, wherein the binding elements selectively bind the protein RGMB.
30. The protein binding device of item 1, wherein the binding elements selectively bind a protein selected from GDF15, FSTL3, FRZB (sFRP3), and BMP4.
31. The protein binding device of item 1, wherein the binding elements are proteins.
32. The protein binding device of item 1, wherein the binding elements are polymers.
33. The protein binding device of item 1, wherein the binding elements are aptamers.
34. The protein binding device of item 1, wherein the binding elements are SOMAmers.
35. The protein binding device of item 1, wherein the binding elements are peptides.
36. The protein binding device of item 1, wherein the binding elements are viruses.
37. The protein binding device of item 1, wherein the binding elements are small molecules.
38. The protein binding device of item 1, wherein the binding elements are nanoparticles.
39. The protein binding device of item 1, wherein the binding element is an antibody.
40. The protein binding device of item 39, wherein the antibody is selected from a monoclonal antibody, polyclonal antibody, humanized monoclonal antibody, human monoclonal antibody, human polyclonal antibody or a humanized polyclonal antibody.
41. The protein binding device of any one of the items 2 to 30, wherein the binding elements are proteins.
42. The protein binding device of any one of the items 2 to 30, wherein the binding elements are polymers.
43. The protein binding device of any one of the items 2 to 30, wherein the binding elements are aptamers.
44. The protein binding device of any one of the items 2 to 30, wherein the binding elements are SOMAmers.
45. The protein binding device of any one of the items 2 to 30, wherein the binding elements are peptides.
46. The protein binding device of any one of the items 2 to 30, wherein the binding elements are viruses.
47. The protein binding device of any one of the items 2 to 30, wherein the binding elements are small molecules.
48. The protein binding device of any one of the items 2 to 30, wherein the binding elements are nanoparticles.
49. The protein binding device of any one of the items 2 to 30, wherein the binding elements is an antibody.
50. The protein binding device of item 49, wherein the antibody is selected from a monoclonal antibody, polyclonal antibody, humanized monoclonal antibody, human monoclonal antibody, human polyclonal antibody or a humanized polyclonal antibody.
51. The protein binding device of item 1, wherein the binding elements selectively bind the protein with a KD of less than 1×10-6 M.
52. The protein binding device of item 51, wherein the binding elements selectively bind the protein with a KD of less than 1×10-7 M.
53. The protein binding device of item 52, wherein the binding elements selectively bind the protein with a KD of less than 1×10-9 M.
54. The protein binding device of any one of the items 2 to 40, wherein the binding elements selectively bind the protein with a KD of less than 1×10-6 M.
55. The protein binding device of item 54, wherein the binding elements selectively bind the protein with a KD of less than 1×10-7 M.
56. The protein binding device of item 55, wherein the binding elements selectively bind the protein with a KD of less than 1×10-9 M.
57. The protein binding device of any one of items 41 to 50, wherein the binding elements selectively bind the protein with a KD of less than 1×10-6 M.
58. The protein binding device of item 57, wherein the binding elements selectively bind the protein with a KD of less than 1×10-7 M.
59. The protein binding device of item 58, wherein the binding elements selectively bind the protein with a KD of less than 1×10-9 M.
60. The protein binding device of item 1, wherein the lumen further comprises a stationary phase, preferably wherein the binding elements are bound to said stationary phase.
61. The protein binding device of item 60, wherein the stationary phase comprises a gel matrix.
62. The protein binding device of item 60, wherein the stationary phase comprises a solid matrix.
63. The protein binding device of item 61, wherein the binding elements are bound to a stationary phase.
64. The protein binding device of item 60, wherein the binding elements are covalently bound to the stationary phase.
65. The protein binding device of item 60, wherein the binding elements are electrostatically bound to the stationary phase.
66. The protein binding device of item 1, wherein the lumen of the housing has a fluid volume capacity of 10 mL or greater.
67. The protein binding device of item 1, wherein the lumen of the housing has a fluid volume capacity of 50 mL or greater.
68. The protein binding device of any one of item 60, wherein the lumen of the housing has a fluid volume capacity of 10 mL or greater.
69. The protein binding device of item 60, wherein the lumen of the housing has a fluid volume capacity of 50 mL or greater.
70. The protein binding device of item 60, wherein the lumen of the housing has a fluid volume capacity of 100 mL or greater.
71. The protein binding device of item 1, wherein binding elements are in the amount of more than 5 pmol.
72. The protein binding device of item 1, wherein binding elements are in the amount of more than 10 pmol.
73. The protein binding device of item 1, wherein binding elements are in the amount of more than 20 pmol.
74. The protein binding device of item 1, wherein binding elements are in the amount of more than 50 pmol.
75. The protein binding device of item 1, wherein binding elements are in the amount of more than 100 pmol.
76. The protein binding device of item 1, wherein binding elements are in the amount of more than 0,3 μM
77. The protein binding device of item 1, wherein binding elements are in the amount of more than 0,5 μM
78. The protein binding device of item 1, wherein binding elements are in the amount of more than 1 μM.
79. The protein binding device of item 1, wherein binding elements are in the amount of more than 5 μM
80. The protein binding device of item 1, wherein binding elements are in the amount of more than 10 μM.
81. The protein binding device of item 1, wherein inlet port and outlet port is the same port, configured to allow passing a fluid though the device.
82. The protein binding device of any one of items 2 to 40, 51-53, wherein the binding elements are bound to a stationary phase.
83. The protein binding device of item 82, wherein the stationary phase comprises a gel matrix.
84. The protein binding device of item 82, wherein the stationary phase comprises a solid matrix.
85. The protein binding device of any one of the item 82, wherein the binding elements are covalently bound to the stationary phase.
86. The protein binding device of item 82, wherein the binding elements are electrostatically bound to the stationary phase.
87. The protein binding device of item 82, wherein the lumen of the housing has a fluid volume capacity of 10 mL or greater.
88. The protein binding device of item 82, wherein the lumen of the housing has a fluid volume capacity of 50 mL or greater.
89. The protein binding device of any one of item 82, wherein the lumen of the housing has a fluid volume capacity of 10 mL or greater.
90. The protein binding device of item 82, wherein the lumen of the housing has a fluid volume capacity of 50 mL or greater.
91. The protein binding device of item 82, wherein the lumen of the housing has a fluid volume capacity of 100 mL or greater.
92. The protein binding device of item 82, wherein binding elements are in the amount of more than 5 pmol.
93. The protein binding device of item 82, wherein binding elements are in the amount of more than 10 pmol.
94. The protein binding device of item 82, wherein binding elements are in the amount of more than 20 pmol.
95. The protein binding device of item 82, wherein binding elements are in the amount of more than 50 pmol.
96. The protein binding device of item 82, wherein binding elements are in the amount of more than 100 pmol.
97. The protein binding device of item 82, wherein binding elements are in the amount of more than 0,3 μM
98. The protein binding device of item 82, wherein binding elements are in the amount of more than 0,5 μM
99. The protein binding device of item 82, wherein binding elements are in the amount of more than 1 μM.
100. The protein binding device of item 82, wherein binding elements are in the amount of more than 5 μM 101. The protein binding device of item 82, wherein binding elements are in the amount of more than 10 μM.
102. The protein binding device of item 82, wherein inlet port and outlet port is the same port, configured to allow passing a fluid though the device.
103. The protein binding device of any one of items 1 to 59, wherein the binding elements are bound to a stationary phase.
104. The protein binding device of item 103, wherein the stationary phase comprises a gel matrix.
105. The protein binding device of item 103, wherein the stationary phase comprises a solid matrix.
106. The protein binding device of any one of items 103 to 105, wherein the binding elements are covalently bound to the stationary phase.
107. The protein binding device of any one of items 103 to 105, wherein the binding elements are electrostatically bound to the stationary phase.
108. The protein binding device of any one of items 1 to 107, wherein the lumen of the housing has a fluid volume capacity of 10 mL or greater.
109. The protein binding device of item 108, wherein the lumen of the housing has a fluid volume capacity of 50 mL or greater.
110. The protein binding device of item 109, wherein the lumen of the housing has a fluid volume capacity of 100 mL or greater.
111. The protein binding device of any one of items 1 to 110, wherein the inlet port of the housing is configured to receive plasma from a whole blood separator.
112. The protein binding device of item 111, wherein the whole blood separator comprises a centrifuge.
113. The protein binding device of any one of items 1 to 112, wherein the device is configured to be operably linked to a pump for advancing fluid into the inlet port of the housing.
114. The protein binding device of any one of items 1 to 113, wherein the device is configured for use without a pump.
115. The protein binding device of item 114, wherein the device is configured for gravitational flow of fluid through the housing.
116. The protein binding device of any one of items 1 to 115, further comprising a first filter element positioned to filter fluid released from the housing.
117. The protein binding device of item 116, wherein the first filter element is situated in the outlet port.
118. The protein binding device of any one of items 1 to 117, further comprising a second filter element positioned to filter fluid entering the housing.
119. The protein binding device of item 118, wherein the second filter element is situated in the inlet port.
120. The protein binding device of any one of items 1 to 119, wherein the housing comprises at least one flexible material.
121. The protein binding device of any one of items 1 to 120, wherein the housing comprises at least one rigid material.
122. The protein binding device of any one of items 1 to 121, wherein the device is a column.
123. A protein binding device, wherein the protein binding device, wherein the device comprises binding elements, wherein the binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein binding elements are in the amount of more than 5 pmol.
124. The protein binding device of item 123, wherein binding elements are in the amount of more than 10 pmol.
125. The protein binding device of item 123, wherein binding elements are in the amount of more than 20 pmol.
126. The protein binding device of item 123, wherein binding elements are in the amount of more than 50 pmol.
127. The protein binding device of item 123, wherein binding elements are in the amount of more than 100 pmol.
128. The protein binding device of item 123, wherein binding elements are in the amount of more than 0,3 μM
129. The protein binding device of item 123, wherein binding elements are in the amount of more than 0,5 μM
130. The protein binding device of item 123, wherein binding elements are in the amount of more than 1 μM.
131. The protein binding device of item 123, wherein binding elements are in the amount of more than 5 μM
132. The protein binding device of item 123, wherein binding elements are in the amount of more than 10 μM.
133. A protein binding device, wherein the protein binding device comprises a housing, wherein the housing comprises binding elements, wherein the binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein a lumen of housing has a fluid volume capacity of 10 mL or greater.
134. The protein binding device of item 133, wherein the lumen of the housing has a fluid volume capacity of 20 mL or greater.
135. The protein binding device of any one of item 134, wherein the lumen of the housing has a fluid volume capacity of 50 mL or greater.
136. The protein binding device of item 135, wherein the lumen of the housing has a fluid volume capacity of 100 mL or greater.
137. The protein binding device of item 136, wherein the lumen of the housing has a fluid volume capacity of 200 mL or greater.
138. A kit comprising the protein binding device of item 1 and instructions for using the device to filter the protein from the plasma of a subject in need thereof.
139. A kit comprising the protein binding device of any one of items 2 to 40, 51-53, 123-137 and instructions for using the device to filter the protein from the plasma of a subject in need thereof.
140. A kit comprising the protein binding device of any one of items 1 to 137, and instructions for using the device to filter the protein from the plasma of a subject in need thereof.
141. A protein binding device, comprising a platform associated with a first group of binding elements and a second group of binding elements, wherein each of the first group and second group of binding elements selectively bind a protein selected from the group: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein the first group of binding elements are selective for a different protein than the second group of binding elements.
142. The protein binding device of item 141, wherein said association is covalent or electrostatic attachment the first group and second group of binding elements to the platform.
143. The protein binding device of item 141 or 142, wherein the protein binding device is a diagnostic chip.
144. The protein binding device of any one of items 141 to 143, wherein each of the first group of binding elements and the second group of binding elements are selected from antibodies, proteins, polymers, nanoparticles, aptamers, peptides, viruses, and small molecules.
145. The protein binding device of item 144, wherein the binding elements are selected from antibodies.
146. The protein binding device of item 145, wherein the antibodies are selected from monoclonal antibodies, polyclonal antibodies, humanized monoclonal antibodies or humanized polyclonal antibodies.
147. The protein binding device of any one of items 141 to 146, wherein the first binding elements selectively bind the protein with a KD of less than 1×10-6 M.
148. The protein binding device of any one of items 141 to 147, wherein the second binding elements selectively bind the protein with a KD of less than 1×10-6 M.
149. The protein binding device of any one of items 141 to 148, wherein the binding elements selectively bind the protein selected from GDF15, FSTL3, FRZB (sFRP3), and BMP4.
150. A fluid filtration device, wherein the fluid filtration device comprises a housing defining a lumen and a filter element configured to contact and filter fluid introduced to the lumen of the housing, wherein the filter element selectively captures a protein in fluid selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
151. The fluid filtration device of item 150, wherein the device is configured to be operably coupled to a component that generates an electromagnetic field or electromagnetic gradient to advance the fluid through the filter element.
152. The fluid filtration device of item 150 or 151, wherein the device is configured to be operably coupled to a pump to advance the fluid through the filter element.
153. A pharmaceutical composition comprising an agent that binds to, inhibits, or degrades a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB; and at least one pharmaceutically acceptable excipient.
154. The pharmaceutical composition of item 153, wherein the an agent binds to, inhibits, or degrades a protein CCDC80.
155. The pharmaceutical composition of item 153, wherein the an agent binds to, inhibits, or degrades a protein CD59.
156. The pharmaceutical composition of item 153, wherein the an agent binds to, inhibits, or degrades a protein CHRDL1.
157. The pharmaceutical composition of item 153, wherein the an agent binds to, inhibits, or degrades a protein COL18A1.
158. The pharmaceutical composition of item 153, wherein the an agent binds to, inhibits, or degrades a protein CST3.
159. The pharmaceutical composition of item 153, wherein the an agent binds to, inhibits, or degrades a protein DPT.
160. The pharmaceutical composition of item 153, wherein the an agent binds to, inhibits, or degrades a protein EFEMP1.
161. The pharmaceutical composition of item 153, wherein the an agent binds to, inhibits, or degrades a protein FAS.
162. The pharmaceutical composition of item 153, wherein the an agent binds to, inhibits, or degrades a protein FSTL3.
163. The pharmaceutical composition of item 153, wherein the an agent binds to, inhibits, or degrades a protein GAS1.
164. The pharmaceutical composition of item 153, wherein the an agent binds to, inhibits, or degrades a protein GDF15.
165. The pharmaceutical composition of item 153, wherein the an agent binds to, inhibits, or degrades a protein KLK11.
166. The pharmaceutical composition of item 153, wherein the an agent binds to, inhibits, or degrades a protein MMP7.
167. The pharmaceutical composition of item 153, wherein the an agent binds to, inhibits, or degrades a protein the protein NBL1.
168. The pharmaceutical composition of item 153, wherein the an agent binds to, inhibits, or degrades a protein NTN1.
169. The pharmaceutical composition of item 153, wherein the an agent binds to, inhibits, or degrades a protein the protein POSTN.
170. The pharmaceutical composition of item 153, wherein the an agent binds to, inhibits, or degrades a protein PTN.
171. The pharmaceutical composition of item 153, wherein the an agent binds to, inhibits, or degrades a protein RELT.
172. The pharmaceutical composition of item 153, wherein the an agent binds to, inhibits, or degrades a protein SFRP1.
173. The pharmaceutical composition of item 153, wherein the an agent binds to, inhibits, or degrades a protein SMOC1.
174. The pharmaceutical composition of item 153, wherein the an agent binds to, inhibits, or degrades a protein STC1.
175. The pharmaceutical composition of item 153, wherein the an agent binds to, inhibits, or degrades a protein TNFRSF1A.
176. The pharmaceutical composition of item 153, wherein the an agent binds to, inhibits, or degrades a protein UNC5C.
177. The pharmaceutical composition of item 153, wherein the an agent binds to, inhibits, or degrades a protein sFRP-3.
178. The pharmaceutical composition of item 153, wherein the an agent binds to, inhibits, or degrades a protein TNFRSF1B.
179. The pharmaceutical composition of item 153, wherein the an agent binds to, inhibits, or degrades a protein CD55.
180. The pharmaceutical composition of item 153, wherein the an agent binds to, inhibits, or degrades a protein BMP4.
181. The pharmaceutical composition of item 153, wherein the an agent binds to, inhibits, or degrades a protein RGMB.
182. The pharmaceutical composition of item 153, wherein the an agent binds to, inhibits, or degrades a protein a selected from GDF15, FSTL3, FRZB (sFRP3), and BMP4.
183. The pharmaceutical composition of item 153, wherein the agent is protein.
184. The pharmaceutical composition of item 153, wherein the agent is polymer.
185. The pharmaceutical composition of item 153, wherein the agent is aptamer.
186. The pharmaceutical composition of item 153, wherein the agent is SOMAmer.
187. The pharmaceutical composition of item 153, wherein the agent is peptide.
188. The pharmaceutical composition of item 153, wherein the agent is virus.
189. The pharmaceutical composition of item 153, wherein the agent is a small molecule.
190. The pharmaceutical composition of item 153, wherein agent is nanoparticle.
191. The pharmaceutical composition of item 153, wherein agent is an antibody.
192. The pharmaceutical composition of item 153, wherein the antibody is selected from a monoclonal antibody, polyclonal antibody, humanized monoclonal antibody, human monoclonal antibody, human polyclonal antibody or a humanized polyclonal antibody.
193. The pharmaceutical composition of any one of the items 154 to 182, wherein the agent is protein.
194. The pharmaceutical composition of any one of the items 154 to 182, wherein the agent is polymer.
195. The pharmaceutical composition of any one of the items 154 to 182, wherein agent is aptamer.
196. The pharmaceutical composition of any one of the items 154 to 182, wherein the agent is SOMAmer.
197. The pharmaceutical composition of any one of the items 154 to 182, wherein the agent is peptide.
198. The pharmaceutical composition of any one of the items 154 to 182, wherein the agent is virus.
199. The pharmaceutical composition of any one of the items 154 to 182, wherein the agent is small molecule.
200. The pharmaceutical composition of any one of the items 154 to 182, wherein agent is nanoparticle.
201. The pharmaceutical composition of of any one of the items 154 to 182, wherein the agent is an antibody.
202. The pharmaceutical composition of item 201, wherein the antibody is selected from a monoclonal antibody, polyclonal antibody, humanized monoclonal antibody, human monoclonal antibody, human polyclonal antibody or a humanized polyclonal antibody.
203. The pharmaceutical composition of any one of item 153, wherein the agent is an anti-GDF15 human antibody, an anti-FSTL3 human antibody, an anti-BMP4 human antibody, or an anti-FRZB (sFRP3) human antibody.
204. The pharmaceutical composition of item 153, wherein the binding element is bound to a particle.
205. The pharmaceutical composition of any one of items 154 to 192, wherein the binding element is bound to a particle.
206. The pharmaceutical composition of any one of items 193 to 203, wherein the binding element is bound to a particle.
207. The pharmaceutical composition of item 204, wherein the binding element is bound to a particle with a covalent bond.
208. The pharmaceutical composition of item 205, wherein the binding element is bound to a particle with a covalent bond.
209. The pharmaceutical composition of item 206, wherein the binding element is bound to a particle with a covalent bond.
210. The pharmaceutical composition comprising binding element to a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB and the particle, wherein the binding element is bound to the particle.
211. The pharmaceutical composition of item 210, wherein the binding element is bound to a particle with a covalent bond.
212. The pharmaceutical composition of any one of the items 153, 207, 210 or 211 wherein particle is a particle having at least one surface and an binding element immobilized on the surface, wherein: the particle comprises a core subparticle and a plurality of protecting subparticles; the binding element is immobilized on the core subparticle.
213. The pharmaceutical composition of item 205, wherein particle is a particle having at least one surface and an binding element immobilized on the surface, wherein: the particle comprises a core subparticle and a plurality of protecting subparticles; the binding element is immobilized on the core subparticle.
214. The pharmaceutical composition of item 208, wherein particle is a particle having at least one surface and an binding element immobilized on the surface, wherein: the particle comprises a core subparticle and a plurality of protecting subparticles; the binding element is immobilized on the core subparticle.
215. The pharmaceutical composition of item 209, wherein particle is a particle having at least one surface and an binding element immobilized on the surface, wherein: the particle comprises a core subparticle and a plurality of protecting subparticles; the binding element is immobilized on the core subparticle.
216. The pharmaceutical composition, comprising at least one pharmaceutically acceptable excipient and an agent modulating or binding or inhibiting or degrading or activating at least one effector upstream or downstream of at least one of the proteins selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, optionally, wherein such modulation or binding or inhibiting or degrading or activating effects or mimics or effects such protein reduction, inhibition or degradation, and optionally has an anti-aging effect.
217. The pharmaceutical composition of item 216, wherein agent is selected from gene therapy, antibody, protein, polymer, nanoparticle, aptamer, peptide, virus, and small molecule.
218. A pharmaceutical composition comprising a blood plasma fraction, wherein the blood plasma fraction comprises a negligible amount of a protein selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
219. A pharmaceutical composition of item 218, comprising one, two or more biomarkers characteristic of aged blood, and wherein the blood plasma fraction comprises a negligible amount of a protein selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
220. A pharmaceutical composition of item 219, wherein biomarkers characteristic of aged blood is a biomarker characteristic of a biological age, optionally a biological age of the subject, from whose blood such pharmaceutical composition was produced.
221. A pharmaceutical composition of item 220, wherein biomarkers characteristic of aged blood is a biomarker characteristic of a biological age, wherein said biological age is a biological age of more than 40 years.
222. A pharmaceutical composition of item 220, wherein biomarkers characteristic of aged blood is a biological age of the subject, whose blood such pharmaceutical composition was produced from, is more than 45 years.
223. A pharmaceutical composition of item 220, wherein biomarkers characteristic of aged blood is a biological age is more than 50 years.
224. A pharmaceutical composition of item 220, wherein biomarkers characteristic of aged blood is a biological age is more than 60 years old.
225. A pharmaceutical composition of item 220, wherein biomarkers characteristic of aged blood is a biological age is more than 70 years old.
226. The pharmaceutical composition of item 220, wherein biomarkers characteristic of blood are selected from:glycohemoglobin, lactate dehydrogenase, uric acid, blood lead, homocysteine, vitamin A, fasting glucose, gamma glutamyltransferase (GGT), total cholesterol, Vitamin E, chloride, aspartate aminotransferase (AST), sodium, and 2,2′, 3,4,4′, 5,5′-heptachlorobiphenyl (PCB180), Blood cadmium, Blood lead, Direct HDL-Cholesterol, Albumin, Blood urea nitrogen, Total calcium, Creatinine, Globulin, Serum glucose, Iron, Phosphorus, Total bilirubin, Total protein, Triglycerides, Uric acid, Total Cholesterol, Glycohemoglobin, Alkaline phosphatase, AST: SI, ALT: SI, Bicarbonate: SI, Chloride: SI, GGT: SI, Potassium: SI, Lactate dehydrogenase LDH, Sodium: SI, Osmolality: SI, HB, LY, MCH, MCHC, MCV, MO, MPV, PLT, RBC, WBC.
227. The pharmaceutical composition of any one of items 220 to 226, wherein instead of biological age a chronological age is used.
228. The pharmaceutical composition of any one of items 218 to 226, wherein a negligible amount is less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml or less than 0.01 pg/ml.
229. The pharmaceutical composition of item 227, wherein a negligible amount is less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml or less than 0.01 pg/ml.
230. A pharmaceutical composition comprising a blood plasma fraction, wherein the blood plasma fraction comprises a negligible amount of at least two proteins selected from B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
231. The pharmaceutical composition of item 230, wherein a negligible amount is less than 15,000 ng/ml; less than 10,500 ng/ml; less than 7,350 ng/ml; less than 5,145 ng/ml; less than 3,602 ng/ml; less than 2,521 ng/ml; less than 1,765 ng/ml; less than 1,235 ng/ml; less than 865 ng/ml; less than 605 ng/ml; less than 424 ng/ml; less than 297 ng/ml; less than 208 ng/ml; less than 145 ng/ml; less than 102 ng/ml; less than 71 ng/ml; less than 50 ng/ml; less than 35 ng/ml; less than 24 ng/ml; less than 17 ng/ml; less than 12 ng/ml; less than 8 ng/ml; less than 6 ng/ml; less than 4 ng/ml; less than 3 ng/ml; less than 2 ng/ml; less than 1 ng/ml; less than 0.99 ng/ml; less than 0.69 ng/ml; less than 0.48 ng/ml; less than 0.34 ng/ml; less than 0.24 ng/ml; less than 0.17 ng/ml; less than 0.12 ng/ml; less than 0.08 ng/ml; less than 0.06 ng/ml; less than 0.04 ng/ml; less than 0.03 ng/ml; less than 0.02 ng/ml; less than 0.013 ng/ml; less than 0.009 ng/ml; less than 0.006 ng/ml; less than 0.004 ng/ml; less than 0.003 ng/ml; less than 0.002 ng/ml; less than 0.001 ng/ml; less than 0.0007 ng/ml; less than 0.0005 ng/ml; less than 0.0003 ng/ml; less than 0.0002 ng/ml; less than 0.0001 ng/ml, less than 0.08 pg/ml, less than 0.06 pg/ml, less than 0.04 pg/ml, less than 0.02 pg/ml or less than 0.01 pg/ml.
232. The pharmaceutical composition of anyone of items 218 to 226, 230, 231, wherein the composition further comprises whole cells.
233. The pharmaceutical composition of any one of items 227 to 229 wherein the composition further comprises whole cells.
234. A pharmaceutical composition comprising a blood plasma fraction, wherein the blood plasma fraction comprises a complex of a protein and a binding element of such protein, wherein a protein is selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB and the binding element of such protein is selected from peptide, small molecule, antibody, aptamer, protein, virus, polymer, nanoparticle or particle.
235. A pharmaceutical composition of item 234, wherein the binding of the protein by the binding element is mimicking the reduction of such protein in bloodstream of the subject.
236. A pharmaceutical composition of item 235, wherein the binding of the protein by the binding element is mimicking the reduction of such protein in bloodstream of the subject to negligible amount.
237. A method for reducing the concentration or amount of a protein in the plasma, comprising introducing the plasma to the protein binding device of item 1, wherein the plasma enters the inlet port and contacts the binding elements and is discharged from the outlet port as modified plasma, and, optionally retrieving the modified plasma from the protein binding device wherein the concentration of the protein in the modified plasma is reduced, optionally selectively reduced, relative to the concentration of the protein in the plasma introduced to the protein binding device.
238. A method for reducing the concentration of a protein in the plasma, comprising introducing the plasma to the protein binding device of any one of items 1 to 40 or 51 to 53, wherein the plasma enters the inlet port and contacts the binding elements and is discharged from the outlet port as modified plasma, and, optionally retrieving the modified plasma from the protein binding device wherein the concentration of the protein in the modified plasma is reduced, optionally selectively reduced, relative to the concentration of the protein in the plasma introduced to the protein binding device.
239. A method for reducing the concentration of a protein in the plasma, comprising introducing the plasma to the protein binding device of any one of items 60 to 81 or 123 to 137, wherein the plasma contacts the binding elements and is discharged from the device as modified plasma, and, optionally retrieving the modified plasma from the protein binding device wherein the concentration of the protein in the modified plasma is reduced, optionally selectively reduced, relative to the concentration of the protein in the plasma introduced to the protein binding device.
240. A method for reducing the concentration of proteins in the plasma, comprising introducing the plasma to the protein binding device of item 141, wherein the plasma contacts the first group and second group of binding elements and is discharged from the protein binding device as modified plasma, and, optionally retrieving the modified plasma from the protein binding device wherein the concentration of the proteins in the modified plasma is reduced, optionally selectively reduced, relative to the concentration of the proteins in the plasma introduced to the protein binding device.
241. A method for reducing the concentration of proteins in the plasma, comprising introducing the plasma to the protein binding device of item 142, wherein the plasma contacts the first group and second group of binding elements and is discharged from the protein binding device as modified plasma, and, optionally retrieving the modified plasma from the protein binding device wherein the concentration of the proteins in the modified plasma is reduced, optionally selectively reduced, relative to the concentration of the proteins in the plasma introduced to the protein binding device.
242. A method for reducing the concentration of a protein in the plasma, comprising introducing the plasma to the plasma filtration device of item 150, wherein the plasma is introduced to the lumen of the housing and contacts the filter element and is discharged from the plasma filtration device as modified plasma, and, optionally, retrieving the modified plasma from the plasma filtration device wherein the concentration of the protein in the modified plasma is reduced, optionally selectively reduced, relative to the concentration of the protein in the plasma introduced to the plasma filtration device.
243. A method for reducing the concentration of a protein in the plasma, comprising introducing the plasma to the device of any one one of preceding items, wherein the plasma is introduced to the lumen of the housing and contacts the filter element and is discharged from the such device as modified plasma, and, optionally, retrieving the modified plasma from the such device wherein the concentration of the protein in the modified plasma is reduced, optionally selectively reduced, relative to the concentration of the protein in the plasma introduced to the such device.
244. A method for reducing the concentration of a protein in plasma, comprising contacting plasma with protein binding elements that selectively bind a protein selected from: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, and, optionally, collecting modified plasma following the contacting step, wherein the modified plasma has a reduced concentration of the protein relative to the concentration of the protein in the plasma prior to the contacting.
245. The method of any one of items 238, or 241 to 244, wherein the protein is reduced, optionally selectively reduced, by about 10% or more.
246. The method of any one of items 238, or 241 to 244, wherein the protein is reduced, optionally selectively reduced, by about 30% or more.
247. The method of any one of items 238, or 241 to 244, wherein the protein is reduced, optionally selectively reduced, by about 70% or more.
248. The method of any one of items 223, or 226 to 228, wherein the protein is reduced, optionally selectively reduced, by about 90% or more.
249. The method of any one of items 238, or 241 to 245, wherein the protein is reduced, optionally selectively reduced, by about 99% or more.
250. The method of any one of items 239 to 240, wherein the protein is reduced, optionally selectively reduced, by about 10% or more.
251. The method of any one of items 239 to 240, wherein the protein is reduced, optionally selectively reduced, by about 30% or more.
252. The method of any one of items 239 to 240, wherein the protein is reduced, optionally selectively reduced, by about 50% or more.
253. The method of any one of items 239 to 240, wherein the protein is reduced, optionally selectively reduced, by about 70% or more.
254. The method of any one of items 239 to 240, wherein the protein is reduced, optionally selectively reduced, by about 90% or more.
255. The method of any one of items 239 to 240, wherein the protein is reduced, optionally selectively reduced, by about 99% or more.
256. The method of any one of items 239 to 240, wherein the protein is reduced, optionally selectively reduced, by about 99,9% or more.
257. The method of item 238, wherein the method further comprises separating plasma from whole blood prior to the contacting.
258. The method of any one of items 241 to 245, wherein the method further comprises separating plasma from whole blood prior to the contacting.
259. The method of any one of items 239 to 240, 251 to 257, wherein the method further comprises separating plasma from whole blood prior to the contacting.
260. The method of item 238, wherein the protein binding element is associated with a protein binding column.
261. The method of any one of items 241 to 245, or 258 wherein the protein binding element is associated with a protein binding column.
262. The method of item 259, wherein the protein binding element is associated with a protein binding column.
263. The method of any one of items 237, 239 to 240, 251 to 257, or 260 wherein the protein binding element is associated with a protein binding column.
264. The method of item 238, wherein the contacting comprises passing the blood plasma fraction through a protein binding column.
265. The method of any one of the items 241 to 245, 259, 261 to 262 or 264 wherein the contacting comprises passing the blood plasma fraction through a protein binding column.
266. A method of treatment or preventing an age-related disease or disorder or other anti-aging treatment comprising deleting, reducing, binding, inhibiting or degrading protein of a protein, selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in fluid of subject.
267. A method of item 266 wherein fluid is blood.
268. A method of item 266, wherein fluid is serum.
269. A method of item 266, wherein fluid is plasma.
270. A method of item 266, wherein reduction is made by protein binding device.
271. A method of item 266 wherein reduction is made by sorption column.
272. A method of any one of the items 266 to 270, wherein reduction is made by protein binding device.
273. A method of any one of the items 266 to 270, wherein reduction is made by sorption column.
274. A method of item 266, wherein reduction is made by filtering device.
275. A method of anyone of the items 266 to 270, wherein reduction is made by filtering device.
276. A method of any one of the items 266 to 270, wherein reduction is made by the device of any one of preceding items.
277. A method of treatment, comprising reducing the concentration or amount of a protein in the fluid of subject by method of item 238.
278. The method of treatment, comprising reducing the concentration of a protein in the plasma by method of any one of the items 241 to 245, or 258 or 265.
279. The method of treatment, comprising reducing the concentration of a protein in the plasma by method of any one of the items 241 to 245, 259, 261 to 262, or 264 or 265.
280. The method of item 278, wherein treatment is treatment or preventing an age-related disease or disorder or other anti-aging treatment.
281. The method of item 279, wherein treatment is treatment or preventing an age-related disease or disorder or other anti-aging treatment.
282. The method of item 279, wherein treatment is treatment or preventing an age-related disease or disorder or other anti-aging treatment.
283. A method of item 266, comprising administering by subject a medication and/or wherein reduction is achieved by administering by subject a medication.
284. A method of item 266, comprising extracorporeal reduction procedure.
285. A method of item 266, comprising intracorporeal reduction procedure.
286. A method of item 266, comprising administering by subject a device.
287. A method of item 266, comprising application of device for reduction of respective protein.
288. A method of treatment or preventing an age-related disease or disorder or other anti-aging treatment comprising administering to a subject in need thereof a pharmaceutical composition of item 153.
289. A method of treatment or preventing an age-related disease or disorder or other anti-aging treatment comprising administering to a subject in need thereof a pharmaceutical composition of any one of items 154-192, 203, 204, 207, 210, 211.
290. A method of treatment or preventing an age-related disease or disorder or other anti-aging treatment comprising administering to a subject in need thereof a pharmaceutical composition of any of preceding items.
291. A method of item any one of the items 288 to 289, wherein composition is administered in therapeutically effective amount.
292. A method of item 290, wherein composition is administered in therapeutically effective amount.
293. A method of item 291, wherein composition is administered in therapeutically effective amount
294. A method of item 266, comprising administering by a subject a gene therapy.
295. Method of treatment by administration on agent binding protein selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein a biological action of such agent is mimicking or effecting the reduction of respective protein in bloodstream of the subject.
296. A method of treating or preventing an age-related disease or disorder or other anti-aging treatment comprising administering to a subject in need thereof a binding element, a modulator, an inhibitor, a degradation agent of effector upstream or downstream of a protein, selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB
297. The method of item 296, wherein biological action of such binding element, a modulator, an inhibitor, a degradation agent mimics a reduction of a protein, selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in bloodstream of the subject.
298. The method of any one of the items 297 to 298 wherein such binding element, modulator, inhibitor, or degradation agent is selected from peptide, small molecule, antibody, aptamer, protein, virus, polymer, gene therapy, nanoparticle or particle.
299. The method of items 297 wherein such binding element, modulator, inhibitor, or degradation agent affects the target protein by contacting an at least one such effector.
300. A method of treating or preventing an age-related disease or disorder or other anti-aging treatment comprising reduction of effector upstream or downstream of a protein, selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
301. A method of item 300, wherein such reduction mimics the reduction of a protein, selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
302. A method of anyone of items 300 to 301, comprising use of protein binding device.
303. A method of item 302, wherein protein binding device comprises a housing, wherein the housing defines a lumen and the housing further comprises an inlet port for receiving fluid to the lumen, and an outlet port for releasing fluid from the lumen, wherein the lumen comprises binding elements, wherein the binding elements selectively bind a protein selected from of effectors upstream or downstream of a protein, selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
304. A method of any one of items 300 to 301, comprising use of fluid filtering device.
305. Use of the device of any of preceding item for blood purification.
306. Use of the device of any of preceding item for modifying plasma for further infusion into the subject in need for.
307. Use of protein binding device of item 1 for reduction a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
308. Use of protein binding device of any one of items 1 to 40 or 51 to 53 for reduction a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
309. Use of device from any one of preceding items for reduction a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
310. Use of protein binding device, comprising binding elements for anti-aging treatment.
311. Use of item 310, wherein the binding elements are proteins.
312. Use of item 310, wherein the binding elements are polymers.
313. Use of item 310, wherein the binding elements are aptamers.
314. Use of item 310, wherein the binding elements are SOMAmers.
315. Use of item 310, wherein the binding elements are peptides.
316. Use of item 310, wherein the binding elements are viruses.
317. Use of item 310, wherein the binding elements are small molecules.
318. Use of item 310, wherein the binding elements are nanoparticles.
319. Use of item 310, wherein the binding elements comprise an antibody.
320. Use of item 319, wherein the antibody is selected from a monoclonal antibody, polyclonal antibody, humanized monoclonal antibody, human monoclonal antibody, human polyclonal antibody or a humanized polyclonal antibody.
321. Use of protein binding device of any one of items 1 to 40 or 51 to 53 for treatment.
322. Use of protein binding device of any one of preceding items for treatment.
323. Use of protein binding device of any one of items 1 to 40 or 51 to 53 for anti-aging treatment.
324. Use of protein binding device of any one of items 60 to 81, or 123 to 138, or 141 or 142, or 150 or 151 for anti-aging treatment.
325. Use of protein binding device comprising binding elements, wherein the binding elements selectively bind at least one effector upstream or downstream of protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB for anti-aging treatment, wherein treatment by such device mimics reduction in bloodstream of subject of protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
326. Use of item 325, wherein protein binding device comprised binding elements selected from peptide, small molecule, antibody, aptamer, protein, virus, polymer, nanoparticle or particle.
327. Use of protein binding device by method of any preceding items.
328. Use of fluid filtering device by method of any preceding items.
329. Use of an inhibitor, a degradation agent or binding element of protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB for treatment.
330. Use of item 329, wherein such inhibitor, a degradation agent or binding element is selected from peptide, small molecule, antibody, aptamer, protein, virus, polymer, nanoparticle or particle.
331. Use of any one of the items 329 to 330, wherein treatment is an anti-aging treatment.
332. Use of composition of any one of items 41 to 56 as a treatment.
333. Use of composition of any one of items 41 to 56 as an anti-aging treatment.
334. Use of composition of any one of preceding items for treatment.
335. Use of composition of any one of preceding items for anti-aging treatment.
336. Use of binding element of protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB for manufacturing of protein binding device.
337. Use of item 336, wherein such device comprises a housing, wherein the housing defines a lumen and the housing further comprises an inlet port for receiving fluid to the lumen, and an outlet port for releasing fluid from the lumen, wherein the lumen comprises such binding element.
338. Use of any one of the items 336 to 337, wherein binding element is selected from peptide, small molecule, antibody, aptamer, protein, virus, polymer, nanoparticle or particle.
339. Kit, comprising pharmaceutical composition of any one of preceding items and instruction for administering it.
340. Kit, comprising pharmaceutical composition of any one of preceding items and instruction for using it as an anti-aging treatment.
341. Kit, comprising pharmaceutical composition of item 153 and instruction for using it for administration by subject for reduction or modulating or binding or inhibiting or degrading of a protein selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB or optionally an effector upstream or downstream of at least one of such protein.
342. Kit, comprising pharmaceutical composition of any of preceding items or selected from described in this application or its structural or functional analog and instruction for using it for administration by subject for reduction or modulating or binding or inhibiting or degrading of a protein selected from CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB or optionally an effector upstream or downstream of at least one of such protein.
343. Kit, comprising binding element or pharmaceutical composition of any of preceding items and instruction for using it for it for treatment.
344. Kit, comprising binding element or pharmaceutical composition of any of preceding items and instruction for using it for it for anti-aging treatment.
345. A kit comprising the device of any of preceding items and instructions for using the device to filter the protein from the plasma of a subject in need thereof.
346. A kit comprising the device of any of preceding items and instructions for using the device for treatment.
347. A kit comprising the device of any of preceding items and instructions for using the device for anti-aging treatment.
348. A tangible medium, configured with instructions that when executed cause a processor to perform a method, the method comprising: attribution to the information about a patient an information about a treatment related to deleting, reducing, binding, inhibiting or degrading protein, selected of the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
349. A tangible medium, configured with instructions that when executed cause a processor to perform a method, the method comprising: attribution to the information about a patient an information about a treatment related to deleting, reducing, binding, inhibiting, activating or degrading of at least one of effectors upstream or downstream of protein, selected of the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
350. A tangible medium of any one of the items 348 and 349, wherein treatment is anti-aging treatment.
351. A tangible medium of any one of the items 348 and 349, wherein such reducing is achieved by protein binding device or by fluid filtering device
352. A tangible medium of any one of the items 349 and 350, wherein such reducing is achieved by device of any one of items 1-40.
353. A tangible medium of any one of the items 349 and 350 or 351, wherein such reducing is achieved by device of any one of preceding items.
354. A tangible medium of item 351, wherein such reducing is achieved by protein binding device or by fluid filtering device
355. A tangible medium of item 351, wherein such reducing is achieved by device of any one of items 1-40.
356. A tangible medium of any one of the items 349 and 350 such deleting, reducing, binding, inhibiting or degrading is achieved by pharmaceutical composition.
357. A tangible medium of item 349 wherein binding is achieved by pharmaceutical composition, comprising binding element of the protein, selected of the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
358. A tangible medium of item 357, wherein binding element is selected from peptide, small molecule, antibody, aptamer, protein, virus, polymer, nanoparticle or particle.
359. A tangible medium, configured with instructions that when executed cause a processor to perform a method, the method comprising: attribution to the information about a patient an information about a treatment related to deleting, reducing, binding, inhibiting or degrading protein, selected of the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein binding is achieved by pharmaceutical composition of any one of the items of 153 to 192.
360. A tangible medium of item 359, wherein binding is achieved by pharmaceutical composition of any one of preceding items.
361. A tangible medium of any one of items 349 to 360, further comprising attributing to the information about patient before or after or before and after the treatment to information about checking of at least one selected from the group: biological age of the patient, at least one aging biomarker, at least one age related deficit or disease, at least one of rejuvenation marker, marker of frailty, marker of health span or marker of life expectancy.
362. A tangible medium comprising a computer program, which, when executed, causes a device to perform a method comprising: attribution to the information regarding protein binding device or any other device or therapeutic agent an information about deleting, reducing, binding, inhibiting or degrading at least one of the proteins, selected of the group of CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
363. A tangible medium of item 362, wherein information about deleting, reducing, binding, inhibiting or degrading respective protein is associated with the information about treatment.
364. A tangible medium of item 362, wherein information about deleting, reducing, binding, inhibiting or degrading respective protein is associated with the information about anti-aging treatment.
365. A tangible medium of any one of the items 349 to 353, 355, 356, 358,359, 362 to 364, wherein tangible medium is a machine readable medium.
366. A tangible medium of any one of preceding items, wherein tangible medium is a machine readable medium.
367. A tangible medium of any one of the items 349 to 353, 355, 356, 358,359, 362 to 366, wherein tangible medium is a computer.
368. A tangible medium of any one of preceding items, wherein tangible medium is a computer.
369. A method of manufacturing of a protein binding device, wherein the protein binding device comprises a housing, wherein the housing defines a lumen and the housing further comprises an inlet port for receiving fluid to the lumen, and an outlet port for releasing fluid from the lumen, comprising a step of linking binding elements to the lumen or any other part of device, wherein the binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
370. A method of item 369, wherein binding element is selected from peptide, small molecule, antibody, aptamer, protein, virus, polymer, nanoparticle or particle.
371. A method of manufacturing of a protein binding device, wherein the protein binding device is described in any one of preceding items, comprising a step of linking binding elements to the lumen or stationary phase or any other part of device, wherein the binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
372. Device, kit, method, pharmaceutical composition, use, medium of any one of preceding items, wherein binding element, binding element or agent comprises a binding element or other molecule described in this application or its structural or functional analog or the molecule or other object comprising the binding fragment of binding element or other molecule described in this application, wherein such fragment binds the protein selected from the group selected of the group of CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB of the part structurally similar to such binding fragment.
373. The method of any one of the items 237 to 265 wherein the reducing is made by the device of any one of preceding items.
374. A method, comprising a reducing the concentration of a protein in plasma of any one of preceding items, further comprising a step of “re-infusing the fluid to the patient/subject”.
375. A protein binding device of any of preceding items, further comprising a platform associated with a first group of binding elements and a second group of binding elements, wherein each of the first group and second group of binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein the first group of binding elements are selective for a different protein than the second group of binding elements.
376. A protein binding device, comprising a platform associated with a first group of binding elements and a second group of binding elements, wherein each of the first group and second group of binding elements selectively bind a protein selected from the group: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein the first group of binding elements are selective for a different protein than the second group of binding elements, further comprising a feature described in any one of preceding items.
377. Plasma obtained or obtainable by the method of any one of items 238 to 265 or by the use of any one of items 305 to 308.
378. Plasma obtained or obtainable by the device of any one of preceding items.
379. Plasma obtained or obtainable by pharmaceutical composition of any one of preceding items.
380. Plasma obtained or obtainable by pharmaceutical composition of any one of preceding items after such composition will be administered by the subject.
381. A binding element for use in the treatment or prevention of an age-related disease or disorder, said treatment comprising passing fluid of a subject over or through the device as defined in any one of preceding items and reinfusing the fluid to the patient, wherein the binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
382. A binding element for use in anti-ageing treatment or prevention, said treatment comprising passing fluid of a subject over or through the device as defined in any one of preceding items and reinfusing the fluid to the patient, wherein the binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB 383. A binding element for use in the treatment or prevention of an age-related disease or disorder, said treatment comprising passing fluid of a subject over or through the device as defined in any one of preceding items and reinfusing the fluid to the patient, wherein the binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein binding element is described in any one of preceding items.
384. A binding element for use in anti-ageing treatment or prevention, said treatment comprising passing fluid of a subject over or through the device as defined in any one of preceding items and reinfusing the fluid to the patient, wherein the binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein binding element is described in any one of preceding items.
385. The binding element for use of any one of preceding items, wherein the binding element is bound to a stationary phase as defined in any one of preceding items.
386. The binding element for use of any one of preceding items, wherein the binding element is comprised in a device as defined in any one of preceding items.
387. A composition comprising (as active agent) a binding element of any one of preceding items or described in application wherein the composition is for use in the anti-aging treatment or prevention.
388. A composition comprising (as active agent) a binding element of any one of preceding items or described in application wherein the composition is for use in the treatment or prevention of an age-related disease or disorder.
389. A composition of any one of the items 387 or 388, wherein the composition is a pharmaceutical composition.
390. A stationary phase as defined in any one of preceding to which a binding element is bound (as active agent), wherein the binding element is for use in the anti-aging treatment or prevention.
391. A stationary phase as defined in any one of preceding to which a binding element is bound (as active agent), wherein the binding element is for use in in the treatment or prevention of an age-related disease or disorder.
392. A device as defined in any one of preceding items comprising a stationary phase to which a binding element is bound (as active agent), wherein the binding element is for use in the anti-aging treatment or prevention of [wording: see items above]
393. A device as defined in any one of preceding items comprising a stationary phase to which a binding element is bound (as active agent), wherein the binding element is for use in the treatment or prevention of an age-related disease or disorder.
394. The binding element for use of any one of preceding items, the composition for use of any one of preceding items or the stationary phase for use of any one of preceding items or the device for use of any one of preceding items, wherein the treatment or prevention further comprises passing fluid of a subject over or through the device under conditions which effect the binding of said binding element to a protein selected from selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
395. The binding element for use of any one of preceding items, the composition for use of any one of preceding items or the stationary phase for use of any one of preceding items or the device for use of any one of preceding items, wherein thereby the protein/a significant portion of the protein is removed from the subject's fluid.
396. The binding element for use of any one of preceding items, the composition for use of any one of preceding items or the stationary phase for use of any one of preceding items or the device for use of any one of preceding items, wherein the concentration/amount of the protein is reduced compared to reference/control/amount in the fluid of the patient prior to the start of the treatment.
397. The binding element for use of any one of preceding items, the composition for use of any one of preceding items or the stationary phase for use of any one of preceding items or the device for use of any one of preceding items, wherein concentration to the protein in the plasma after the passing the plasma over the device is at least 30% less than before the passing.
398. The use or treatment of any one of preceding items further comprising obtaining fluid from the subject.
399. The binding element for use of any one of preceding items, the composition for use of any one of preceding items or the stationary phase for use of any one of preceding items or the device for use of any one of preceding items, the method of any one of preceding items, wherein the subject suffers from an age-related disease or disorder.
400. The binding element for use of any one of preceding items, the composition for use of any one of preceding items or the stationary phase for use of any one of preceding items or the device for use of any one of preceding items, the method of any one of preceding items, wherein the subject suffers from ageing/symptoms of ageing.
401. The binding element for use of any one of preceding items, the composition for use of any one of preceding items or the stationary phase for use of any one of preceding items or the device for use of any one of preceding items, the method of any one of preceding items, wherein the subject suffers from ageing/symptoms of ageing selected from the group:
402. A binding element as defined in any one of preceding items or in this application for use in the treatment or prevention of an age-related disease or disorder.
403. A binding element as defined in any one of preceding items or in this application for use in the anti-aging treatment or prevention.
404. Modified plasma as defined in any one of preceding items or pharmaceutical composition, comprising plasma fraction as defined in any one of preceding items or plasma obtained or obtainable by the method of any one of preceding items.
405. Modified plasma as defined in any one of preceding items or pharmaceutical composition, comprising plasma fraction as defined in any one of preceding items for use in the treatment or prevention an age-related disease or disorder.
406. Modified plasma as defined in any one of preceding items or pharmaceutical composition, comprising plasma fraction as defined in any one of preceding items for use in the anti-aging treatment or prevention.
407. Composition comprising (as active agent) a binding element as defined in any one of preceding items or as defined in this application or composition comprising (Modified) plasma (as active agent) as defined in any one of items or as defined in this application or composition comprising plasma obtained or obtainable by the method of any one of preceding items, wherein the composition is for use in the anti-aging treatment or prevention.
408. Composition comprising (as active agent) a binding element as defined in any one of preceding items or as defined in this application or composition comprising (Modified) plasma (as active agent) as defined in any one of items or as defined in this application or composition comprising plasma obtained or obtainable by the method of any one of preceding items, wherein the composition is for use in the treatment or prevention an age-related disease or disorder.
409. Composition or binding element of any one of preceding items, wherein the binding element is for use in the anti-aging treatment or prevention or wherein the composition is for use in the anti-aging treatment or prevention.
410. Composition or binding element of any one of preceding items, wherein the binding element is for use in the anti-aging treatment or prevention or wherein the composition is for use in the treatment or prevention an age-related disease or disorder.
411. Composition or binding element of any one of preceding items, the binding element for use of any one of preceding items, the composition for use of any one of preceding items or the stationary phase for use of any one of preceding items or the device for use of any one of preceding items, the method of any one of preceding items, wherein said treatment comprises passing fluid of a subject over or the device as defined in any one of preceding items.
412. Composition or binding element of anyone of preceding items, the binding element for use of any one of preceding items, the composition for use of any one of preceding items or the stationary phase for use of any one of preceding items or the device for use of any one of preceding items, the method of any one of preceding items, further comprising reinfusing the fluid to the patient.
413. Composition of any one of preceding items, wherein said composition is a pharmaceutical composition.
414. Use of a binding element as defined in any one of preceding items or in this application or of (Modified) plasma as defined in any one of preceding items or in this application or of plasma obtained or obtainable by the method of any one of preceding items or of a composition comprising said binding element or said plasma as an anti-ageing agent.
415. Use of a binding element as defined in any one of preceding items or in this application or of (Modified) plasma as defined in any one of preceding items or in this application or of plasma obtained or obtainable by the method of any one of preceding items or of a composition comprising said binding element or said plasma as an anti-ageing agent in a subject.
416. Use of a binding element as defined in any one of preceding items or in this application or of (Modified) plasma as defined in any one of preceding items or in this application or of plasma obtained or obtainable by the method of any one of preceding items or of a composition comprising said binding element or said plasma for maintaining or improving health of a subject.
417. Use of a binding element as defined in any one of preceding items or in this application or of (Modified) plasma as defined in any one of preceding items or in this application or of plasma obtained or obtainable by the method of any one of preceding items or of a composition comprising said binding element or said plasma for maintaining or improving fitness of a subject.
418. Use of a binding element as defined in any one of preceding items or in this application or of (Modified) plasma as defined in any one of preceding items or in this application or of plasma obtained or obtainable by the method of any one of preceding items or of a composition comprising said binding element or said plasma for improving/increasing activity in a subject.
419. Use of a binding element as defined in any one of preceding items or in this application or of (Modified) plasma as defined in any one of preceding items or in this application or of plasma obtained or obtainable by the method of any one of preceding items or of a composition comprising said binding element or said plasma for improving muscle strength, bone density, hair growth, cognitive performance, stress resistance or resilience, blood parameters, heart rate, cognitive functions, basal metabolic rate, systolic blood pressure, heel bone mineral density (BMD), heel quantitative ultrasound index (QUI), heel broadband ultrasound attenuation, heel broadband ultrasound attenuation, forced expiratory volume in 1-second (FEV1), forced vital capacity (FVC), peak expiratory flow (PEF), duration to first press of snap-button in each round, reaction time, mean time to correctly identify matches, hand grip strength (right and/or left), whole body fat-free mass, leg fat-free mass (right and/or left), time for recovery after any stress (wound, operation, chemotherapy, disease, change in lifestyle etc.), Standing height, Forced expiratory volume in 1-second (FEV1), Leg fat-free mass (right), Leg predicted mass (right), Basal metabolic rate, Forced vital capacity (FVC), Leg fat-free mass (left), Leg predicted mass (left), Systolic blood pressure, automated reading, Heel bone mineral density (BMD) (left), Heel quantitative ultrasound index (QUI), direct entry (left), Whole body fat-free mass, Whole body water mass, Heel bone mineral density (BMD) T-score, automated (left), Speed of sound through heel (left), Sitting height, Heel bone mineral density (BMD) (right), Heel quantitative ultrasound index (QUI), direct entry (right), Speed of sound through heel (right), Heel bone mineral density (BMD) T-score, automated (right), Peak expiratory flow (PEF), Leg fat percentage (left), Trunk fat-free mass, Leg fat percentage (right), Trunk predicted mass, Hand grip strength (left), Heel broadband ultrasound attenuation (left), Heel broadband ultrasound attenuation (right), Hand grip strength (right), Duration to first press of snap-button in each round, Mean time to correctly identify matches, Body fat percentage, Trunk fat percentage, Body mass index (BMI), Leg fat mass (left), Arm fat-free mass (left), Arm predicted mass (left), Arm fat-free mass (right), Haematocrit percentage, Arm predicted mass (right), Waist circumference, Leg fat mass (right), Haemoglobin concentration, Arm fat percentage (left), Ankle spacing width (left), Whole body fat mass, Body mass index (BMI), Pulse wave peak to peak time, Arm fat percentage (right), Weight, Mean corpuscular volume, Trunk fat mass, Pulse wave Arterial Stiffness index, Ankle spacing width (right), Platelet crit, Red blood cell (erythrocyte) count, Mean sphered cell volume, Mean platelet (thrombocyte) volume, Weight, Arm fat mass (left), Lymphocyte percentage, Neutrophill percentage, Arm fat mass (right), Impedance of leg (left), Mean reticulocyte volume, Platelet count, Mean corpuscular haemoglobin, Impedance of leg (right), Red blood cell (erythrocyte) distribution width, Pulse rate, automated reading, Impedance of whole body, Diastolic blood pressure, automated reading, Lymphocyte count, Number of measurements made, Neutrophill count, Monocyte percentage, Hip circumference, Monocyte count, Platelet distribution width, Mean corpuscular haemoglobin concentration, Immature reticulocyte fraction, Impedance of arm (right), Reticulocyte percentage, Number of times snap-button pressed, White blood cell (leukocyte) count, Pulse rate, High light scatter reticulocyte count, Basophill percentage, Impedance of arm (left), Pulse wave reflection index, Eosinophill count, Nucleated red blood cell count, Eosinophill percentage, Basophill count, Reticulocyte count, High light scatter reticulocyte percentage, Nucleated red blood cell percentage, or any one other parameter worsening with the age.
420. Use of any one of preceding items of a binding element as defined in any one of preceding items or in this application or of (Modified) plasma as defined in any one of preceding items or in this application or of plasma obtained or obtainable by the method of any one of preceding items or of a composition comprising said binding element or said plasma, wherein said subject is a healthy subject.
421. Use of any one of preceding items of a binding element as defined in any one of preceding items or in this application or of (Modified) plasma as defined in any one of preceding items or in this application or of plasma obtained or obtainable by the method of any one of preceding items or of a composition comprising said binding element or said plasma, wherein said subject is an elderly subject
422. Use of any one of preceding items of a binding element as defined in any one of preceding items or in this application or of (Modified) plasma as defined in any one of preceding items or in this application or of plasma obtained or obtainable by the method of any one of preceding items or of a composition comprising said binding element or said plasma, wherein said subject is an subject of an age of >40 years.
423. Use of any one of preceding items of a binding element as defined in any one of preceding items or in this application or of (Modified) plasma as defined in any one of preceding items or in this application or of plasma obtained or obtainable by the method of any one of preceding items or of a composition comprising said binding element or said plasma, wherein said subject is an subject of an age of >50 years.
424. Use of any one of preceding items of a binding element as defined in any one of preceding items or in this application or of (Modified) plasma as defined in any one of preceding items or in this application or of plasma obtained or obtainable by the method of any one of preceding items or of a composition comprising said binding element or said plasma, wherein said subject is an subject of an age of >60 years.
425. Use of any one of preceding items of a binding element as defined in any one of preceding items or in this application or of (Modified) plasma as defined in any one of preceding items or in this application or of plasma obtained or obtainable by the method of any one of preceding items or of a composition comprising said binding element or said plasma, wherein said subject shows symptoms of ageing.
426. Use of any one of preceding items of a binding element as defined in any one of preceding items or in this application or of (Modified) plasma as defined in any one of preceding items or in this application or of plasma obtained or obtainable by the method of any one of preceding items or of a composition comprising said binding element or said plasma, wherein said symptoms of ageing are decline in overall activity, reduced muscle strength, bone degeneration, reduced bone density, hair loss, cognitive decline, worsening of any of the parameters defined in item 419.
427. Use of any one of preceding items of a binding element as defined in any one of preceding items or in this application or of (Modified) plasma as defined in any one of preceding items or in this application or of plasma obtained or obtainable by the method of any one of preceding items or of a composition comprising said binding element or said plasma, wherein said subject does not suffer from an age-related disease or disorder.
428. Use of any one of preceding items wherein said use is a non-therapeutic use.
429. A binding element as defined in any one of preceding items or in this application or a (Modified) plasma as defined in any one of preceding items or in this application or plasma obtained or obtainable by the method of any one of preceding items or a composition comprising said binding element or said plasma for use as a medicament.
430. A binding element as defined in any one of preceding items or in this application or a (Modified) plasma as defined in any one of preceding items or in this application or plasma obtained or obtainable by the method of any one of preceding items or a composition comprising said binding element or said plasma for use in medicine;
431. A binding element as defined in any one of preceding items or in this application or a (Modified) plasma as defined in any one of preceding items or in this application or plasma obtained or obtainable by the method of any one of preceding items or a composition comprising said binding element or said plasma for use in a method for treatment of the human or animal body by therapy (optionally practiced on the human or animal body).
432. A method for detecting the presence and/or concentration of proteins in the plasma of a subject in need thereof, comprising introducing the plasma to the protein binding device of any one of items 141 to 142 and detecting the presence or amount of protein bound to the first group of binding elements and/or second group of binding elements.
433. A method for detecting the presence and/or concentration of proteins in the plasma of a subject in need thereof, comprising introducing the plasma to the protein binding device of any one of preceding items and detecting the presence or amount of protein bound to the first group of binding elements and/or second group of binding elements.
434. A stationary phase, to which the binding elements are bound, wherein the binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
435. A stationary phase, to which the binding elements are bound wherein the binding elements are described in any one of preceding items.
436. A stationary phase of any one of the items 434 to 435, wherein stationary phase is sorbent.
437. A method of manufacturing of stationary phase, comprising a step of linking binding elements to the stationary phase, wherein the binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
438. A method of manufacturing of device of any preceding items, comprising a step of linking binding elements to the device or to stationary phase of such device, wherein the binding elements selectively bind a protein selected from the group: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.
439. A protein binding device of any of preceding items, wherein the binding element is bound to a particle, wherein particle either attached the device or is preserved in device or extracted from the fluid after it is bound with the protein.
440. Device, kit, method, composition, use, a binding element, medium, (modified) plasma or stationary phase of any one of preceding items, wherein fluid is blood.
441. Device, kit, method, composition, use, a binding element, medium, (modified) plasma or stationary phase of any one of preceding items, wherein fluid is serum.
442. Device, kit, method, composition, use, a binding element, medium, (modified) plasma or stationary phase of any one of preceding items, wherein fluid fluid is plasma.
443. Device, kit, method, composition, use, a binding element, medium, (modified) plasma or stationary phase of any one of preceding items, wherein instead of plasma a serum is used.
444. Device, kit, method, composition, use, a binding element, medium, (modified) plasma or stationary phase of any one of preceding items, wherein instead of plasma a blood is used.
445. Device, kit, method, composition, use, a binding element, medium, (modified) plasma or stationary phase of any one of preceding items, wherein the subject is a human.
446. Device, kit, method, composition, use, a binding element, medium, (modified) plasma or stationary phase of any one of preceding items, wherein the subject is a human of an age >40 years.
447. Device, kit, method, composition, use, a binding element, medium, (modified) plasma or stationary phase of any one of preceding items, wherein the subject is a human of an age >50 years.
448. Device, kit, method, composition, use, a binding element, medium, (modified) plasma or stationary phase of any one of preceding items, wherein the subject is a human of an age >60 years.
449. Device, kit, method, composition, use, a binding element, medium, (modified) plasma or stationary phase of any one of preceding items, wherein the subject is a human of a biological age >40 years.
450. Device, kit, method, composition, use, a binding element, medium, (modified) plasma or stationary phase of any one of preceding items, wherein the subject is a human of a biological age >50 years.
451. Device, kit, method, composition, use, a binding element, medium, (modified) plasma or stationary phase of any one of preceding items, wherein the subject is a human of a biological age >60 years.
452. Device, kit, method, composition, use, a binding element, medium, (modified) plasma or stationary phase of any one of preceding items, wherein subject is an elderly human.
453. Device, kit, method, composition, use, a binding element, medium, (modified) plasma or stationary phase of any one of preceding items, wherein subject is a male.
454. Device, kit, method, composition, use, a binding element, medium, (modified) plasma or stationary phase of any one of preceding items, wherein subject is a female.
455. Device, kit, method, composition, use, a binding element, medium, (modified) plasma or stationary phase of any one of preceding items, wherein subject suffers from an age-related disease or disorder.
456. Device, kit, method, pharmaceutical composition, use, a binding element, medium of any one of preceding items wherein said age-related disease or disorder is selected from the group: frailty, Alzheimer's, Parkinson's, and Huntington's diseases, cardiovascular disease, renal failure, muscle wasting [cachexia], osteopenia or osteoporosis, obesity, insulin resistance or diabetes, and diverse adult-onset cancers, atherosclerosis, cardiovascular disease, adult cancer, arthritis, cataracts, osteoporosis, type 2 diabetes, hypertension, neurodegeneration (including but not limited to Alzheimer's disease, Huntington's disease, and other age-progressive dementias; Parkinson's disease; and amyotrophic lateral sclerosis [ALS]), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence (including but not limited to age related decline in immune response to vaccines, age related decline in response to immunotherapy etc.), myocardial infarction, acute coronary syndrome, sarcopenia, sarcopenic obesity, senile osteoporosis, urinary incontinence or any other age related disease, including but not limited to those described in this application or in the sources referenced in this application.
457. A protein binding device of any of preceding items, wherein the characteristics that “the protein binding device comprises a housing, wherein the housing defines a lumen and the housing further comprises an inlet port for receiving fluid to the lumen, and an outlet port for releasing fluid from the lumen, wherein the lumen comprises binding elements” is optional, and wherein such device comprises binding elements are in the amount of as defined in any one of the items 123 to 132.
458. A protein binding device of any one of preceding items, wherein binding elements are in the amount of as defined in any one of the items 123 to 132.
459. The protein binding device of any one of the items 1 to 122, wherein binding elements are in the amount as defined in any one of the items 123 to 132.
460. A device of any one of preceding items, wherein a housing has a fluid volume capacity as defined in any one of the items 133 to 137.
461. A protein binding device of any one of preceding items, wherein the characteristics that “the housing further comprises an inlet port for receiving fluid to the lumen, and an outlet port for releasing fluid from the lumen”, is optional, and wherein a housing has a fluid volume capacity as defined in any one of the items 133 to 137.
462. A device of any one of items 103 to 110, wherein the binding elements are in the amount as defined in any one of items 92 to 101.
463. The protein binding device of item 141 or 142, further comprising feature of device, which described in any one of preceding items.
464. A protein binding device, use, method or kit, comprising protein binding device of any one of preceding items, wherein stationary phase is a sorbent.
465. Device, kit, method, composition, use, a binding element, medium, (modified) plasma or stationary phase of any one of preceding items, wherein the binding element is defined in any one of preceding items or in this application.
466. The device of one of preceding items, further comprising feature of device, which described in any one of preceding items.
467. Composition comprising a binder to GDF-15 for use as medicament.
468. A binder to GDF-15 for use as medicament.
469. Composition comprising a binder to GDF-15, including but not limited to those described in this application for use in the anti-aging treatment, excluding cachexia
470. Composition comprising a binder to GDF-15 for use in the anti-aging treatment, excluding cachexia.
471. Composition comprising a binder to GDF-15 including but not limited to those described in this application for use in the of treatment an age-related disease or disorder, excluding cachexia.
472. Composition or use of any one of preceding items, wherein GDF-15 binder is AV-380 antibody
473. The pharmaceutical composition of item 216, further comprising a feature described in any one of the items 183 to 215.
474. Device, kit, method, composition, use, a binding element, medium, (modified) plasma or stationary phase of any one of preceding items, further comprising a feature described in any one of the items 183 to 215.
475. Kit comprising one or more binding elements as defined in the preceding items, a housing as defined in any of the preceding items and, optionally, a stationary phase as defined in any of the preceding items, optionally with instructions to prepare a device as defined in any of the preceding items.
476. Kit, comprising device, kit, method, composition, use, a binding element, medium, (modified) plasma or stationary phase of any one of preceding items and instruction for using it in treatment.
477. Kit, comprising device, kit, method, composition, use, a binding element, medium, (modified) plasma or stationary phase of any one of preceding items and instruction for using the device to filter the protein from the plasma of a subject in need thereof.
478. Kit, comprising device, kit, method, composition, use, a binding element, medium, (modified) plasma or stationary phase of any one of preceding items and instruction for using it in anti-aging treatment.
479. Kit, comprising device, kit, method, composition, use, a binding element, medium, (modified) plasma or stationary phase of any one of preceding items and instruction for using it in treatment of an age-related disease or disorder.
480. Kit, comprising device, kit, method, composition, use, a binding element, medium, (modified) plasma or stationary phase of any one of preceding items and instruction for using it in treatment or prevention of disease, decline, condition or disorder described in in any one of preceding items or in the description.
481. A device, kit, method, composition, use, a binding element, medium, (modified) plasma or stationary phase of any one of preceding items wherein binding element comprises the molecule described in such item, e.g. if the invention is described as the “protein binding device of item 1, wherein the binding element is an antibody” for the purposes of the embodiment of this item 481 is read as the invention described as the protein binding device of item 1, wherein the binding element comprises an antibody and correspondently is if “binding elements are small molecules” for purposes of embodiment of this item 481 the invention is described as “binding element comprises small molecule” as well “binding element comprises small molecules”
482. A device of one of preceding items, further comprising feature of device, which described in any one of preceding items.
483. A device, kit, method, composition, use, a binding element, medium, (modified) plasma or stationary phase of any one of preceding items wherein age-related disease or disorder is selected from sarcopenia, menopausal syndrome, atherosclerosis, cardiovascular disease, adult cancer, arthritis, cataracts, osteoporosis, type 2 diabetes, hypertension, neurodegeneration (including but not limited to Alzheimer's disease, Huntington's disease, and other age-progressive dementias; Parkinson's disease; and amyotrophic lateral sclerosis [ALS]), stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence (including but not limited to age related decline in immune response to vaccines, age related decline in response to immunotherapy etc.), myocardial infarction, acute coronary syndrome, sarcopenia, sarcopenic obesity, senile osteoporosis, urinary incontinence, neuromuscular disorder, osteoarthritis, chronic fatigue syndrome, senile dementia, mild cognitive impairment due to aging, Creutzfeldt-Jakob disease, stroke, CNS cerebral senility, age-related cognitive decline, pre-diabetes, diabetes, obesity, osteoporosis, coronary artery disease, cerebrovascular disease, heart attack, stroke, peripheral arterial disease, aortic valve disease, stroke, Lewy body disease, amyotrophic lateral sclerosis (ALS), mild cognitive impairment, pre-dementia, dementia, progressive subcortical gliosis, progressive supranuclear palsy, thalamic degeneration syndrome, hereditary aphasia, myoclonus epilepsy, macular degeneration, frailty, pressure ulcers, delirium or any other age related disease, including but not limited to those described in this application or in the sources referenced in this application.
484. Device, kit, method, composition, use, a binding element, medium, (modified) plasma or stationary phase of any one of preceding items, for use in reducing morbidity or mortality risks in said subject.
485. Device, kit, method, composition, use, a binding element, medium, (modified) plasma or stationary phase of any one of preceding items, wherein subject shows aging-related change in any parameter or physiological metric, including but not limited to aging related change in blood parameters, heart rate, cognitive functions/decline, bone density, basal metabolic rate, systolic blood pressure, heel bone mineral density (BMD), heel quantitative ultrasound index (QUI), heel broadband ultrasound attenuation, heel broadband ultrasound attenuation, forced expiratory volume in 1-second (FEV1), forced vital capacity (FVC), peak expiratory flow (PEF), duration to first press of snap-button in each round, reaction time, mean time to correctly identify matches, hand grip strength (right and/or left), whole body fat-free mass, leg fat-free mass (right and/or left), and time for recovery after any stress (wound, operation, chemotherapy, disease, change in lifestyle etc.) and others, including but not limited to those mentioned in item 419.
486. Device, kit, method, composition, use, a binding element, medium, (modified) plasma or stationary phase of any one of preceding items, wherein the subject suffers from a decline in one or more parameters characteristic for ageing.
487. Device, kit, method, composition, use, a binding element, medium, (modified) plasma or stationary phase of any one of preceding items, wherein the subject suffers from a disease or a condition or decline selected from those described in anti-aging treatment definition in this application.
488. Device, kit, method, composition, use, a binding element, medium, (modified) plasma or stationary phase of any one of preceding items, wherein binding element is selected from described in this application or is its structural or functional analog.
489. Device, kit, composition, a binding element, or stationary phase of any one of preceding items, wherein such device, kit, composition, a binding element, or stationary phase comprises binding element selected from described in this application or is its structural or functional analog.
490. Device, kit, method, composition, use, a binding element, medium, (modified) plasma or stationary phase of any one of preceding items, wherein reduction of protein is made by the device, kit, method, composition, use, a binding element, medium, (modified) plasma or stationary phase of any one of preceding items 491. Any one of the preceding items, wherein instead of device of item 1 at least one other device described in this disclosure is used.
492. Any one of the preceding items, wherein instead of method described in such item at least one other method described in this disclosure is used.
493. Any one of the preceding items, wherein instead of kit described in such item at least one other kit described in this disclosure is used.

Moreover, the present disclosure also relates to the following items:

1. A protein binding device, wherein the protein binding device comprises a housing, wherein the housing defines a lumen and the housing further comprises an inlet port for receiving fluid to the lumen, and an outlet port for releasing fluid from the lumen, wherein the lumen comprises binding elements, wherein the binding elements selectively bind a protein selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

2. A pharmaceutical composition provided as part of an anti-aging treatment or for treating or preventing an age-related disease or disorder comprising an agent configured to bind to, inhibit, or degrade a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB; and at least one pharmaceutically acceptable excipient.

3. The pharmaceutical composition of Item 2, wherein the protein is FSTL3, wherein the pharmaceutical composition is devoid of or excludes compounds or molecules for treating insulin resistance, diastolic heart failure, and obesity.

4. The pharmaceutical composition of Item 2, wherein the agent is selected from the group consisting of: a protein, a polymer, an aptamer, a SOMAmer, a peptide, a virus, a small molecule, a nanoparticle, an antibody, a monoclonal antibody, a polyclonal antibody, a humanized monoclonal antibody, a human monoclonal antibody, a human or humanized polyclonal antibody, an anti-GDF15 human antibody, an anti-FSTL3 human antibody, an anti-BMP4 human antibody, and an anti-FRZB (sFRP3) human antibody.

5. The pharmaceutical composition of Item 4, wherein the agent is bound to a particle.

6. A pharmaceutical composition comprising a blood plasma fraction, wherein the blood plasma fraction comprises a negligible amount of a protein selected from the group consisting of CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

7. The pharmaceutical composition of Item 6, wherein the negligible amount is between about 0.008 pg/mL and about 15,000 ng/mL.

8. The pharmaceutical composition of Item 6, comprising one or more biomarkers characteristic of aged blood.

9. The pharmaceutical composition of Item 8, wherein one of the biomarkers characteristic of aged blood is a biological age of the subject from whose blood the pharmaceutical composition is produced, wherein the biological age of the subject is greater than 45 years of age.

10. A method of providing an anti-aging treatment or of treating or preventing an age-related disease or disorder of a subject comprising reducing, inhibiting, or degrading a protein selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in the blood of the subject.

11. The method of Item 10, further comprising administering to the subject a gene therapy.

12. The method of Item 10, wherein the age-related disease or disorder is associated with an alleviated level of a protein selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in a bodily fluid of the subject.

13. The method of Item 10, wherein the age-related disease or disorder is selected from the group consisting of frailty, Alzheimer's disease, Parkinson's disease, Huntington's diseases, cardiovascular disease, renal failure, muscle wasting or cachexia, osteopenia or osteoporosis, obesity, insulin resistance or diabetes, diverse adult-onset cancers, atherosclerosis, cardiovascular disease, adult cancer, arthritis, cataracts, osteoporosis, type 2 diabetes, hypertension, age-progressive dementia; amyotrophic lateral sclerosis, stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, age related decline in immune response to vaccines, age related decline in response to immunotherapy, myocardial infarction, acute coronary syndrome, sarcopenia, sarcopenic obesity, senile osteoporosis, urinary incontinence, stroke, atrophic gastritis, camptocormia, chronic obstructive pulmonary disease, coronary artery disease, dopamine dysregulation syndrome, late life depression, osteoarthritis, chronic fatigue syndrome, senile dementia, mild cognitive impairment due to aging, Creutzfeldt-Jakob disease, stroke, CNS cerebral senility, pre-diabetes, diabetes, peripheral arterial disease, aortic valve disease, stroke, Lewy body disease, progressive subcortical gliosis, progressive supranuclear palsy, thalamic degeneration syndrome, hereditary aphasia, myoclonus epilepsy, macular degeneration, pressure ulcers, delirium, progressive subcortical gliosis, progressive supranuclear palsy, thalamic degeneration syndrome, hereditary aphasia, myoclonus epilepsy, and metabolic disorder.

14. The method of Item 10, wherein the anti-aging treatment is selected from the group consisting of a treatment leading to prevention, amelioration or lessening the effects of aging; decreasing or delaying an increase in a biological age of the subject; slowing a rate of aging of the subject; prevention, amelioration or lessening the effects of frailty; prevention, amelioration or lessening the effects of at least one of an aging-related disease or conditions; increasing a health span or lifespan of the subject; increasing a stress resistance or resilience of the subject; increasing a rate or other enhancement of recovery after surgery, radiotherapy, disease and/or any other stress; prevention, amelioration or lesion the effects of menopausal syndrome; restoring reproductive function; elimination or lessening the spread of senescent cells; modulation of at least one biomarker of aging; a decrease in a rate of wrinkle development; and decrease in a rate of hair greying.

15. The method of Item 10, wherein the anti-aging treatment is selected from the group consisting of a treatment related to changing a blood parameter, a heart rate, a cognitive function, a bone density, a basal metabolic rate, a systolic blood pressure, a heel bone mineral density (BMD), a heel quantitative ultrasound index (QUI), a heel broadband ultrasound attenuation, a forced expiratory volume in 1-second (FEV1), forced vital capacity (FVC), a peak expiratory flow (PEF), a duration to first press of snap-button in each round, a reaction time, a mean time to correctly identify matches, a right or left hand grip strength, a whole body fat-free mass, a leg fat-free mass, a time for recovery after a stress-inducing event, a resistance to radiation, a morbidity risk, and a mortality risk of the subject.

16. A method of providing an anti-aging treatment or of treating or preventing an age-related disease or disorder of a subject, comprising administering to the subject a pharmaceutical composition comprising an inhibitor of a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB; and at least one pharmaceutically acceptable excipient.

17. A method of providing an anti-aging treatment or of treating or preventing an age-related disease or disorder of a subject, comprising administering to the subject a pharmaceutical composition comprising a blood plasma fraction, wherein the blood plasma fraction comprises a negligible amount of a protein selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein the negligible amount is between about 0.008 pg/mL and about 15,000 ng/mL.

18. A kit, comprising: a pharmaceutical composition comprising: an agent configured to bind to, inhibit, or degrade a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein the agent is selected from the group consisting of: a protein, a polymer, an aptamer, a SOMAmer, a peptide, a virus, a small molecule, a nanoparticle, an antibody, a monoclonal antibody, a polyclonal antibody, a humanized monoclonal antibody, a human monoclonal antibody, a human or humanized polyclonal antibody, an anti-GDF15 human antibody, an anti-FSTL3 human antibody, an anti-BMP4 human antibody, and an anti-FRZB (sFRP3) human antibody, and at least one pharmaceutically acceptable excipient; and an instruction for using the pharmaceutical composition as part of the anti-aging treatment or to treat or prevent an age-related disease or disorder.

19. A tangible medium comprising a computer program, which, when executed by one or more processors of a computing device, causes the computing device to attribute information regarding a protein binding device, another device, or a therapeutic agent with information about reducing, binding, inhibiting, or degrading at least one protein within the blood of the subject, wherein the protein is selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, and wherein the information about reducing, binding, inhibiting or degrading the protein is associated with an anti-aging treatment.

20. The tangible medium of Item 19, wherein the tangible medium is a computer storage medium.

Tables

TABLE 1

| Field | Units |
|---|---|
| Standing height | cm |
| Forced expiratory volume in 1-second (FEV1) | litres |
| Leg fat-free mass (right) | Kg |
| Leg predicted mass (right) | Kg |
| Basal metabolic rate | KJ |
| Forced vital capacity (FVC) | litres |
| Leg fat-free mass (left) | Kg |
| Leg predicted mass (left) | Kg |
| Systolic blood pressure, automated reading | mmHg |
| Heel bone mineral density (BMD) (left) | g/cm2 |
| Heel quantitative ultrasound index (QUI), | |
| direct entry (left) | |
| Whole body fat-free mass | Kg |
| Whole body water mass | Kg |
| Heel bone mineral density (BMD) T-score, | Std. Devs |
| automated (left) | |
| Speed of sound through heel (left) | m/s |
| Sitting height | cm |
| Heel bone mineral density (BMD) (right) | g/cm2 |
| Heel quantitative ultrasound index (QUI), | |
| direct entry (right) | |
| Speed of sound through heel (right) | m/s |
| Heel bone mineral density (BMD) T-score, | Std. Devs |
| automated (right) | |
| Peak expiratory flow (PEF) | litres/min |
| Leg fat percentage (left) | percent |
| Trunk fat-free mass | Kg |
| Leg fat percentage (right) | percent |
| Trunk predicted mass | Kg |
| Hand grip strength (left) | Kg |
| Heel broadband ultrasound attenuation (left) | dB/MHz |
| Heel broadband ultrasound attenuation (right) | dB/MHz |
| Hand grip strength (right) | Kg |
| Duration to first press of snap-button in each | milliseconds |
| round | |
| Mean time to correctly identify matches | milliseconds |
| Body fat percentage | percent |
| Trunk fat percentage | percent |
| Body mass index (BMI) | Kg/m2 |
| Leg fat mass (left) | Kg |
| Arm fat-free mass (left) | Kg |
| Arm predicted mass (left) | Kg |
| Arm fat-free mass (right) | Kg |
| Haematocrit percentage | percent |
| Arm predicted mass (right) | Kg |
| Waist circumference | cm |
| Leg fat mass (right) | Kg |
| Haemoglobin concentration | grams/decilitre |
| Arm fat percentage (left) | percent |
| Ankle spacing width (left) | mm |
| Whole body fat mass | Kg |
| Body mass index (BMI) | Kg/m2 |
| Pulse wave peak to peak time | milliseconds |
| Arm fat percentage (right) | percent |
| Weight | Kg |
| Mean corpuscular volume | femtolitres |
| Trunk fat mass | Kg |
| Pulse wave Arterial Stiffness index | |
| Ankle spacing width (right) | mm |
| Platelet crit | percent |
| Red blood cell (erythrocyte) count | 10\hat{ }12 |
| | cells/Litre |
| Mean sphered cell volume | femtolitres |
| Mean platelet (thrombocyte) volume | femtolitres |
| Weight | Kg |
| Arm fat mass (left) | Kg |
| Lymphocyte percentage | percent |
| Neutrophill percentage | percent |
| Arm fat mass (right) | Kg |
| Impedance of leg (left) | ohms |
| Mean reticulocyte volume | femtolitres |
| Platelet count | 10\hat{ }9 |
| | cells/Litre |
| Mean corpuscular haemoglobin | picograms |
| Impedance of leg (right) | ohms |
| Red blood cell (erythrocyte) distribution width | percent |
| Pulse rate, automated reading | bpm |
| Impedance of whole body | ohms |
| Diastolic blood pressure, automated reading | mmHg |
| Lymphocyte count | 10\hat{ }9 |
| | cells/Litre |
| Number of measurements made | |
| Neutrophill count | 10\hat{ }9 |
| | cells/Litre |
| Monocyte percentage | percent |
| Hip circumference | cm |
| Monocyte count | 10\hat{ }9 |
| | cells/Litre |
| Platelet distribution width | percent |
| Mean corpuscular haemoglobin concentration | grams/decilitre |
| Immature reticulocyte fraction | ratio |
| Impedance of arm (right) | ohms |
| Reticulocyte percentage | percent |
| Number of times snap-button pressed | |
| White blood cell (leukocyte) count | 10\hat{ }9 |
| | cells/Litre |
| Pulse rate | bpm |
| High light scatter reticulocyte count | 10\hat{ }12 |
| | cells/Litre |
| Basophill percentage | percent |
| Impedance of arm (left) | ohms |
| Pulse wave reflection index | |
| Eosinophill count | 10\hat{ }9 |
| | cells/Litre |
| Nucleated red blood cell count | 10\hat{ }9 |
| | cells/Litre |
| Eosinophill percentage | percent |
| Basophill count | 10\hat{ }9 |
| | cells/Litre |
| Reticulocyte count | 10\hat{ }12 |
| | cells/Litre |
| High light scatter reticulocyte percentage | percent |
| Nucleated red blood cell percentage | percent |

### “Declines”.

Non-limiting list of parameters which age related change is regarded as age related decline and which can be changed into younger state or stabilized or its further change into the older state delayed by anti-aging intervention of this disclosure

Additional Examples of GDF15 Binding Elements

Naturally occurring antibodies are multimeric proteins that contain four polypeptide chains. Two of the polypeptide chains are called heavy chains (H chains), and two of the polypeptide chains are called light chains (L chains). The immunoglobulin heavy and light chains are connected by an interchain disulfide bond. The immunoglobulin heavy chains are connected by interchain disulfide bonds. A light chain consists of one variable region VL and one constant region CL. The heavy chain consists of one variable region VH and at least three constant regions CH1, CH2 and CH3. The variable regions determine the specificity of the antibody. Each variable region comprises three hypervariable regions also known as complementarity determining regions (CDRs) flanked by four relatively conserved framework regions (FRs). The three CDRs, referred to as CDR1, CDR2, and CDR3, contribute to the antibody binding specificity. Naturally occurring antibodies have been used as starting material for engineered antibodies, such as chimeric antibodies and humanized antibodies.

### Example 1 A

A monoclonal antibody capable of binding to human GDF-15, or an antigen-binding portion thereof, wherein the heavy chain variable domain comprises a CDR3 region comprising the amino acid sequence of SEQ ID NO: 5 or an amino acid sequence at least 90% identical thereto, and wherein the light chain variable domain comprises a CDR3 region comprising the amino acid sequence of SEQ ID NO: 7 or an amino acid sequence at least 85% identical thereto.

A monoclonal antibody capable of binding to human GDF-15, or an antigen-binding portion thereof, wherein the binding is binding to a conformational or discontinuous epitope on human GDF-15 comprised by the amino acid sequences of SEQ ID No: 25 and SEQ ID No: 26, for use in a method for treating cancer cachexia in a mammal.

SEQ ID NO: 5
ARSSYGAMDY
SEQ ID NO: 7
QQYNNFPYT
SEQ ID NO: 25
EVQVTMCIGACPSQFR
SEQ ID NO: 26
TDTGVSLQTYDDLLAKDCHCI

Examples 1B Exemplary anti-GDF15 antibodies may, for example, include a heavy chain variable region comprising any one of the nine sets of CDRH1, CDRH2, and CDRH3 region sequences set forth in Table 1 immediately below.

Exemplary anti-GDF15 antibodies may, for example, include a light chain variable region comprising any one of the four sets of CDR_{L1}, CDR_{L2}, and CDR_{L3} region sequences set forth in Table 2 immediately below.

TABLE 1

| NO | CDRH1 | CDRH2 | CDRH3 |
|---|---|---|---|
| 1 | DYNMD | QINPNNG | EAITTV |
| | (SEQ ID | GIFFNQK | GAMDY |
| | NO: 1) | FKG | (SEQ ID |
| | | (SEQ ID | NO: 13) |
| | | NO: 4) | |
| 2 | DYNMD | QINPNNG | EAITTV |
| | (SEQ ID | GIFFNQK | GAMDY |
| | NO: 1) | FQG | (SEQ ID |
| | | (SEQ ID | NO: 13) |
| | | NO: 5) | |
| 3 | DYNMD | QINPYNH | EAITT |
| | (SEQ ID | LIFFNQK | VGAM |
| | NO:1) | FQG | DY |
| | | (SEQ ID | (SEQ ID |
| | | NO: 6) | NO: 13) |
| 4 | DYNMD | QINPNNGL | EAITTV |
| | (SEQ ID | IFFNQK | GAMDY |
| | NO: 1) | FQG | (SEQ ID |
| | | (SEQ ID | NO: 13) |
| | | NO: 7) | |
| 5 | DYNMD | QINPNNG | EAITTV |
| | (SEQ ID | LIFFNQK | GAMDY |
| | NO: 1) | FKG | (SEQ ID |
| | | (SEQ ID | NO: 13) |
| | | NO: 8) | |
| 6 | DYNMD | QINPYNH | EAITTV |
| | (SEQ ID | LIFFNQK | GAMDY |
| | NO: 1) | FKG | (SEQ ID |
| | | (SEQ ID | NO: |
| | | NO: 9) | 13) |
| 7 | TYGMG | HIYWDDD | RGYDD |
| | VS | KRYNPSL | YWGY |
| | (SEQ ID | KS | (SEQ ID |
| | NO: 2) | (SEQ ID | NO: 14) |
| | | NO: 10 | |
| 8 | TYGMGVS | HIYWDDD | RGYDD |
| | (SEQ ID NO: | KRYNPSL | YWGY |
| | 2) | KT | (SEQ ID |
| | | (SEQ ID | NO: |
| | | NO: 11) | 14) |
| 9 | TYGM | DIW-WDD | RGHYS |
| | GVG | DKYYNPS | AMDY |
| | (SEQ ID | LKS | (SEQ ID |
| | NO: 3) | (SEQ ID | NO: 15) |
| | | NO: 12) | |

TABLE 2

| NO | CDRL1 | CDRL2 | CDRL3 |
|---|---|---|---|
| 1 | RTSENL | DAKTLAD | QHFWSSPYT |
| | HNYLA | (SEQ ID | (SEQ ID |
| | (SEQ ID | NO: 18) | NO: 21) |
| | NO: 16) | | |
| 2 | RTSENL | DAKTLAD | QHFWSDPYT |
| | HNYLA | (SEQ ID | (SEQ ID |
| | (SEQ ID | NO: 18) | NO: 22) |
| | NO: 16) | | |
| 3 | KASQNV | SASYRYS | QQYNNYPLT |
| | GTNVA | (SEQ ID NO: | (SEQ ID |
| | (SEQ ID NO: | 19) | NO: 23) |
| | 17) | | |
| 4 | KASQNVGTN | SPSYRYS | QQYNSYPHT |
| | VA | (SEQ ID NO: | (SEQ ID |
| | (SEQ ID NO: | 20) | NO: 24) |
| | 17) | | |

Examples 1C (a) (i) an immunoglobulin heavy chain variable region comprising a CDRH1 comprising an amino acid sequence selected from the group consisting of SEQ ID NO:1 and SEQ ID NO:38 (Hu01G06 IGHV1-18 F2), a CDRH2 comprising an amino acid sequence selected from the group consisting of SEQ ID NO:237 and SEQ ID NO:241 (Hu01G06 IGHV1-18 F2), and a CDRH3 comprising the amino acid sequence of SEQ ID NO:15 (Hu01G06 IGHV1-18 F2); and (ii) an immunoglobulin light chain variable region comprising a CDRL1 comprising the amino acid sequence of SEQ ID NO:21 (Hu01G06 IGKV1-39 F2), a CDRL2 comprising the amino acid sequence of SEQ ID NO:26 (Hu01G06 IGKV1-39 F2), and a CDRL3 comprising the amino acid sequence of SEQ ID NO:244 (Hu01G06 IGKV1-39 F2);

(b) (i) an immunoglobulin heavy chain variable region comprising a CDRH1 comprising an amino acid sequence selected from the group consisting of SEQ ID NO:1 and SEQ ID NO:234 (Hu01G06 IGHV1-69 F1), a CDRH2 comprising an amino acid sequence selected from the group consisting of SEQ ID NO:238 and SEQ ID NO:241 (Hu01G06 IGHV1-69 F1), and a CDRH3 comprising the amino acid sequence of SEQ ID NO:15 (Hu01G06 IGHV1-69 F1); and
(ii) an immunoglobulin light chain variable region comprising a CDRL1 comprising the amino acid sequence of SEQ ID NO:21 (Hu01G06 IGKV1-39 F1), a CDRL2 comprising the amino acid sequence of SEQ ID NO:26 (Hu01G06 IGKV1-39 F1), and a CDRL3 comprising the amino acid sequence of SEQ ID NO:32 (Hu01G06 IGKV1-39 F1);
(c) (i) an immunoglobulin heavy chain variable region comprising a CDRH1 comprising the amino acid sequence of SEQ ID NO:1 (01G06, Ch01G06 Chimeric, Hu01G06 IGHV1-18, Hu01G06 IGHV1-69, Sh01G06 IGHV1-18 M69L, Sh01G06 IGHV1-69 T30S 169L), a CDRH2 comprising the amino acid sequence of SEQ ID NO:7 (01G06, Ch01G06 Chimeric, Hu01G06 IGHV1-18, Hu01G06 IGHV1-69, Sh01G06 IGHV1-18 M69L, Sh01G06 IGHV1-69 T30S 169L), and a CDRH3 comprising the amino acid sequence of SEQ ID NO:15 (01G06, Ch01G06 Chimeric, Hu01G06 IGHV1-18, Hu01G06 IGHV1-69, Sh01G06 IGHV1-18 M69L, Sh01G06 IGHV1-69 T30S 169L); and
(ii) an immunoglobulin light chain variable region comprising a CDRL1 comprising the amino acid sequence of SEQ ID NO:21 (01G06, Ch01G06 Chimeric, Hu01G06 IGKV1-39, Hu01G06 IGKV1-39 S43A V481, Hu01G06 IGKV1-39 V481), a CDRL2 comprising the amino acid sequence of SEQ ID NO:26 (01G06, Ch01G06 Chimeric, Hu01G06 IGKV1-39, Hu01G06 IGKV1-39 S43A V481, Hu01G06 IGKV1-39 V481), and a CDRL3 comprising the amino acid sequence of SEQ ID NO:32 (01G06, Ch01G06 Chimeric, Hu01G06 IGKV1-39, Hu01G06 IGKV1-39 S43A V481, Hu01G06 IGKV1-39 V481);
(d) (i) an immunoglobulin heavy chain variable region comprising a CDRH1 comprising the amino acid sequence of SEQ ID NO:2 (03G05), a CDRH2 comprising the amino acid sequence of SEQ ID NO:8 (03G05), and a CDRH3 comprising the amino acid sequence of SEQ ID NO:16 (03G05); and
(ii) an immunoglobulin light chain variable region comprising a CDRL1 comprising the amino acid sequence of SEQ ID NO:22 (03G05), a CDRL2 comprising the amino acid sequence of SEQ ID NO:27 (03G05), and a CDRL3 comprising the amino acid sequence of SEQ ID NO 33 (03G05);
(e) (i) an immunoglobulin heavy chain variable region comprising a CDRH1 comprising the amino acid sequence of SEQ ID NO:3 (04F08), a CDRH2 comprising the amino acid sequence of SEQ ID NO:9 (04F08), and a CDRH3 comprising the amino acid sequence of SEQ ID NO:17 (04F08); and
(ii) an immunoglobulin light chain variable region comprising a CDRL1 comprising the amino acid sequence of SEQ ID NO:23 (04F08), a CDRL2 comprising the amino acid sequence of SEQ ID NO:28 (04F08), and a CDRL3 comprising the amino acid sequence of SEQ ID NO:34 (04F08);
(f) (i) an immunoglobulin heavy chain variable region comprising a CDRH1 comprising the amino acid sequence of SEQ ID NO:4 (06C11, Ch06C11 Chimeric, Hu06C11 IGHV2-5), a CDRH2 comprising the amino acid sequence of SEQ ID NO:9 (06C11, Ch06C11 Chimeric, Hu06C11 IGHV2-5), and a CDRH3 comprising the amino acid sequence of SEQ ID NO:18 (06C11, Ch06C11 Chimeric, Hu06C11 IGHV2-5); and
(ii) an immunoglobulin light chain variable region comprising a CDRL1 comprising the amino acid sequence of SEQ ID NO:23 (06C11, Ch06C11 Chimeric, Sh06C11 IGKV1-16), a CDRL2 comprising the amino acid sequence of SEQ ID NO:28 (06C11, Ch06C11 Chimeric, Sh06C11 IGKV1-16), and a CDRL3 comprising the amino acid sequence of SEQ ID NO:35 (06C11, Ch06C11 Chimeric, Sh06C11 IGKV1-16);
(g) (i) an immunoglobulin heavy chain variable region comprising a CDRH1 comprising the amino acid sequence of SEQ ID NO:1 (08G01), a CDRH2 comprising the amino acid sequence of SEQ ID NO:10 (08G01), and a CDRH3 comprising the amino acid sequence of SEQ ID NO:15 (08G01); and
(ii) an immunoglobulin light chain variable region comprising a CDRL1 comprising the amino acid sequence of SEQ ID NO:24 (08G01), a CDRL2 comprising the amino acid sequence of SEQ ID NO:29 (08G01), and a CDRL3 comprising the amino acid sequence of SEQ ID NO:32 (08G01);
(h) (i) an immunoglobulin heavy chain variable region comprising a CDRH1 comprising the amino acid sequence of SEQ ID NO:5 (14F11, Ch14F11 Chimeric, Sh14F11 IGHV2-5, Sh14F11 IGHV2-70), a CDRH2 comprising the amino acid sequence of SEQ ID NO:11 (14F11, Ch14F11 Chimeric, Sh14F11 IGHV2-5, Sh14F11 IGHV2-70), and a CDRH3 comprising the amino acid sequence of SEQ ID NO:19 (14F11, Ch14F11 Chimeric, Sh14F11 IGHV2-5, Sh14F11 IGHV2-70); and
(ii) an immunoglobulin light chain variable region comprising a CDRL1 comprising the amino acid sequence of SEQ ID NO:23 (14F11, Ch14F11 Chimeric, Hu14F11 IGKV1-16), a CDRL2 comprising the amino acid sequence of SEQ ID NO:30 (14F11, Ch14F11 Chimeric, Hu14F11 IGKV1-16), and a CDRL3 comprising the amino acid sequence of SEQ ID NO:36 (14F11, Ch14F11 Chimeric, Hu14F11 IGKV1-16);
(i) (i) an immunoglobulin heavy chain variable region comprising a CDRH1 comprising the amino acid sequence of SEQ ID NO:6 (17B11), a CDRH2 comprising the amino acid sequence of SEQ ID NO:12 (17B11), and a CDRH3 comprising the amino acid sequence of SEQ ID NO:20 (17B11); and
(ii) an immunoglobulin light chain variable region comprising a CDRL1 comprising the amino acid sequence of SEQ ID NO:25 (17B11), a CDRL2 comprising the amino acid sequence of SEQ ID NO:31 (17B11), and a CDRL3 comprising the amino acid sequence of SEQ ID NO:37 (17B11);
(j) (i) an immunoglobulin heavy chain variable region comprising a CDRH1 comprising the amino acid sequence of SEQ ID NO:1 (Sh01G06 IGHV1-18 M69L K64Q G44S, Sh01G06 IGHV1-18 M69L K64Q, Sh01G06 IGHV1-69 T30S K64Q 169L), a CDRH2 comprising the amino acid sequence of SEQ ID NO:13 (Sh01G06 IGHV1-18 M69L K64Q G44S, Sh01G06 IGHV1-18 M69L K64Q, Sh01G06 IGHV1-69 T30S K64Q 169L), and a CDRH3 comprising the amino acid sequence of SEQ ID NO:15 (Sh01G06 IGHV1-18 M69L K64Q G44S, Sh01G06 IGHV1-18 M69L K64Q, Sh01G06 IGHV1-69 T30S K64Q 169L); and
(ii) an immunoglobulin light chain variable region comprising a CDRL1 comprising the amino acid sequence of SEQ ID NO:21 (01G06, Ch01G06 Chimeric, Hu01G06 IGKV1-39, Hu01G06 IGKV1-39 S43A V481, Hu01G06 IGKV1-39 V481), a CDRL2 comprising the amino acid sequence of SEQ ID NO:26 (01G06, Ch01G06 Chimeric, Hu01G06 IGKV1-39, Hu01G06 IGKV1-39 S43A V481, Hu01G06 IGKV1-39 V481), and a CDRL3 comprising the amino acid sequence of SEQ ID NO:32 (01G06, Ch01G06 Chimeric, Hu01G06 IGKV1-39, Hu01G06 IGKV1-39 S43A V481, Hu01G06 IGKV1-39 V481);
(k) (i) an immunoglobulin heavy chain variable region comprising a CDRH1 comprising an amino acid sequence selected from the group consisting of SEQ ID NO:1 and SEQ ID NO:38 (Hu01G06 IGHV1-18 F1), a CDRH2 comprising an amino acid sequence selected from the group consisting of SEQ ID NO:236 and SEQ ID NO:240 (Hu01G06 IGHV1-18 F1), and a CDRH3 comprising the amino acid sequence of SEQ ID NO:15 (Hu01G06 IGHV1-18 F1); and
(ii) an immunoglobulin light chain variable region comprising a CDRL1 comprising the amino acid sequence of SEQ ID NO:21 (Hu01G06 IGKV1-39 F1), a CDRL2 comprising the amino acid sequence of SEQ ID NO:26 (Hu01G06 IGKV1-39 F1), and a CDRL3 comprising the amino acid sequence of SEQ ID NO:32 (Hu01G06 IGKV1-39 F1);
(l) (i) an immunoglobulin heavy chain variable region comprising a CDRH1 comprising an amino acid sequence selected from the group consisting of SEQ ID NO:1 and SEQ ID NO:234 (Hu01G06 IGHV1-69 F2), a CDRH2 comprising an amino acid sequence selected from the group consisting of SEQ ID NO:239 and SEQ ID NO:240 (Hu01G06 IGHV1-69 F2), and a CDRH3 comprising the amino acid sequence of SEQ ID NO:15 (Hu01G06 IGHV1-69 F2); and
(ii) an immunoglobulin light chain variable region comprising a CDRL1 comprising the amino acid sequence of SEQ ID NO:21 (Hu01G06 IGKV1-39 F1), a CDRL2 comprising the amino acid sequence of SEQ ID NO:26 (Hu01G06 IGKV1-39 F1), and a CDRL3 comprising the amino acid sequence of SEQ ID NO:32 (Hu01G06 IGKV1-39 F1); (m) (i) an immunoglobulin heavy chain variable region comprising a CDRH1 comprising an amino acid sequence selected from the group consisting of SEQ ID NO:1 and SEQ ID NO:234 (Hu01G06 IGHV1-69 F2), a CDRH2 comprising an amino acid sequence selected from the group consisting of SEQ ID NO:239 and SEQ ID NO:240 (Hu01G06 IGHV1-69 F2), and a CDRH3 comprising the amino acid sequence of SEQ ID NO:15 (Hu01G06 IGHV1-69 F2); and
(ii) an immunoglobulin light chain variable region comprising a CDRL1 comprising the amino acid sequence of SEQ ID NO:21 (Hu01G06 IGKV1-39 F2), a CDRL2 comprising the amino acid sequence of SEQ ID NO:26 (Hu01G06 IGKV1-39 F2), and a CDRL3 comprising the amino acid sequence of SEQ ID NO:244 (Hu01G06 IGKV1-39 F2);
(n) (i) an immunoglobulin heavy chain variable region comprising a CDRH1 comprising the amino acid sequence of SEQ ID NO:4 (HE LM 06C11 IGHV2-70), a CDRH2 comprising the amino acid sequence of SEQ ID NO:14 (HE LM 06C11 IGHV2-70), and a CDRH3 comprising the amino acid sequence of SEQ ID NO:18 (HE LM 06C11 IGHV2-70); and
(ii) an immunoglobulin light chain variable region comprising a CDRL1 comprising the amino acid sequence of SEQ ID NO:23 (Ch06C11 Chimeric, Sh06C11 IGKV1-16), a CDRL2 comprising the amino acid sequence of SEQ ID NO:28 (Ch06C11 Chimeric, Sh06C11 IGKV1-16), and a CDRL3 comprising the amino acid sequence of SEQ ID NO:35 (Ch06C11 Chimeric, Sh06C11 IGKV1-16);
(o) (i) an immunoglobulin heavy chain variable region comprising a CDRH1 comprising the amino acid sequence of SEQ ID NO:4 (Ch06C11 Chimeric), a CDRH2 comprising the amino acid sequence of SEQ ID NO:9 (Ch06C11 Chimeric), and a CDRH3 comprising the amino acid sequence of SEQ ID NO:18 (Ch06C11 Chimeric); and
(ii) an immunoglobulin light chain variable region comprising a CDRL1 comprising the amino acid sequence of SEQ ID NO:23 (Ch14F11 Chimeric), a CDRL2 comprising the amino acid sequence of SEQ ID NO:30 (Ch14F11 Chimeric), and a CDRL3 comprising the amino acid sequence of SEQ ID NO:36 (Ch14F11 Chimeric); and
(p) (i) an immunoglobulin heavy chain variable region comprising a CDRH1 comprising the amino acid sequence of SEQ ID NO:5 (Ch14F11 Chimeric), a CDRH2 comprising the amino acid sequence of SEQ ID NO:11 (Ch14F11 Chimeric), and a CDRH3 comprising the amino acid sequence of SEQ ID NO:19 (Ch14F11 Chimeric); and
(ii) an immunoglobulin light chain variable region comprising a CDRL1 comprising the amino acid sequence of SEQ ID NO:23 (Ch06C11 Chimeric), a CDRL2 comprising the amino acid sequence of SEQ ID NO:28 (Ch06C11 Chimeric), and a CDRL3 comprising the amino acid sequence of SEQ ID NO:35 (Ch06C11 Chimeric); and

An isolated antibody that binds human GDF15, comprising an immunoglobulin heavy chain variable region and an immunoglobulin light chain variable region selected from the group consisting of:

(a) an immunoglobulin heavy chain variable region comprising the amino acid sequence of SEQ ID NO:248 (Hu01G06 IGHV1-18 F2), and an immunoglobulin light chain variable region comprising the amino acid sequence of SEQ ID NO:254 (Hu01G06 IGKV1-39 F2);
(b) an immunoglobulin heavy chain variable region comprising the amino acid sequence of SEQ ID NO:250 (Hu01G06 IGHV1-69 F1), and an immunoglobulin light chain variable region comprising the amino acid sequence of SEQ ID NO:92 (Hu01G06 IGKV1-39 F1);
(c) an immunoglobulin heavy chain variable region comprising the amino acid sequence of SEQ ID NO:40 (01G06, Ch01G06 Chimeric), and an immunoglobulin light chain variable region comprising the amino acid sequence of SEQ ID NO:76 (01G06, Ch01G06 Chimeric);
(d) an immunoglobulin heavy chain variable region comprising the amino acid sequence of SEQ ID NO:42 (03G05), and an immunoglobulin light chain variable region comprising the amino acid sequence of SEQ ID NO:78 (03G05);
(e) an immunoglobulin heavy chain variable region comprising the amino acid sequence of SEQ ID NO:44 (04F08), and an immunoglobulin light chain variable region comprising the amino acid sequence of SEQ ID NO:80 (04F08);
(f) an immunoglobulin heavy chain variable region comprising the amino acid sequence of SEQ ID NO:46 (06C11), and an immunoglobulin light chain variable region comprising the amino acid sequence of SEQ ID NO:82 (06C11);
(g) an immunoglobulin heavy chain variable region comprising the amino acid sequence of SEQ ID NO:48 (08G01), and an immunoglobulin light chain variable region comprising the amino acid sequence of SEQ ID NO:84 (08G01);
(h) an immunoglobulin heavy chain variable region comprising the amino acid sequence of SEQ ID NO:50 (14F11), and an immunoglobulin light chain variable region comprising the amino acid sequence of SEQ ID NO:86 (14F11);
(i) an immunoglobulin heavy chain variable region comprising the amino acid sequence of SEQ ID NO:52 (17B11), and an immunoglobulin light chain variable region comprising the amino acid sequence of SEQ ID NO:88 (17B11);
(j) an immunoglobulin heavy chain variable region comprising the amino acid sequence of SEQ ID NO:54 (Hu01G06 IGHV1-18), and an immunoglobulin light chain variable region comprising the amino acid sequence of SEQ ID NO:76 (Ch01G06 Chimeric);
(k) an immunoglobulin heavy chain variable region comprising the amino acid sequence of SEQ ID NO:56 (Hu01G06 IGHV1-69), and an immunoglobulin light chain variable region comprising the amino acid sequence of SEQ ID NO:76 (Ch01G06 Chimeric);
(l) an immunoglobulin heavy chain variable region comprising the amino acid sequence of SEQ ID NO:58 (Sh01G06 IGHV1-18 M69L), and an immunoglobulin light chain variable region comprising the amino acid sequence of SEQ ID NO:76 (Ch01G06 Chimeric);
(m) an immunoglobulin heavy chain variable region comprising the amino acid sequence of SEQ ID NO:60 (Sh01G06 IGHV1-18 M69L K64Q G44S), and an immunoglobulin light chain variable region comprising the amino acid sequence of SEQ ID NO:76 (Ch01G06 Chimeric);
(n) an immunoglobulin heavy chain variable region comprising the amino acid sequence of SEQ ID NO:62 (Sh01G06 IGHV1-18 M69L K64Q), and an immunoglobulin light chain variable region comprising the amino acid sequence of SEQ ID NO:76 (Ch01G06 Chimeric);
(o) an immunoglobulin heavy chain variable region comprising the amino acid sequence of SEQ ID NO:64 (Sh01G06 IGHV1-69 T30S 169L), and an immunoglobulin light chain variable region comprising the amino acid sequence of SEQ ID NO:76 (Ch01G06 Chimeric);
(p) an immunoglobulin heavy chain variable region comprising the amino acid sequence of SEQ ID NO:66 (Sh01G06 IGHV1-69 T30S K64Q 169L), and an immunoglobulin light chain variable region comprising the amino acid sequence of SEQ ID NO:76 (Ch01G06 Chimeric);
(q) an immunoglobulin heavy chain variable region comprising the amino acid sequence of SEQ ID NO:40 (Ch01G06 Chimeric), and an immunoglobulin light chain variable region comprising the amino acid sequence of SEQ ID NO:90 (Hu01G06 IGKV1-39);
(r) an immunoglobulin heavy chain variable region comprising the amino acid sequence of SEQ ID NO:54 (Hu01G06 IGHV1-18), and an immunoglobulin light chain variable region comprising the amino acid sequence of SEQ ID NO:90 (Hu01G06 IGKV1-39);
(s) an immunoglobulin heavy chain variable region comprising the amino acid sequence of SEQ ID NO:56 (Hu01G06 IGHV1-69), and an immunoglobulin light chain variable region comprising the amino acid sequence of SEQ ID NO:90 (Hu01G06 IGKV1-39);
(t) an immunoglobulin heavy chain variable region comprising the amino acid sequence of SEQ ID NO:58 (Sh01G06 IGHV1-18 M69L), and an immunoglobulin light chain variable region comprising the amino acid sequence of SEQ ID NO:90 (Hu01G06 IGKV1-39);
(u) an immunoglobulin heavy chain variable region comprising the amino acid sequence of SEQ ID NO:60 (Sh01G06 IGHV1-18 M69L K64Q G44S), and an immunoglobulin light chain variable region comprising the amino acid sequence of SEQ ID NO:90 (Hu01G06 IGKV1-39);
(v) an immunoglobulin heavy chain variable region comprising the amino acid sequence of SEQ ID NO:62 (Sh01G06 IGHV1-18 M69L K64Q), and an immunoglobulin light chain variable region comprising the amino acid sequence of SEQ ID NO:90 (Hu01G06 IGKV1-39);
(w) an immunoglobulin heavy chain variable region comprising the amino acid sequence of SEQ ID NO:64 (Sh01G06 IGHV1-69 T30S 169L), and an immunoglobulin light chain variable region comprising the amino acid sequence of SEQ ID NO:90 (Hu01G06 IGKV1-39);
(x) an immunoglobulin heavy chain variable region comprising the amino acid sequence of SEQ ID NO:66 (Sh01G06 IGHV1-69 T30S K64Q 169L), and an immunoglobulin light chain variable region comprising the amino acid sequence of SEQ ID NO:90 (Hu01G06 IGKV1-39);
(y) an immunoglobulin heavy chain variable region comprising the amino acid sequence of SEQ ID NO:40 (Ch01G06 Chimeric), and an immunoglobulin light chain variable region comprising the amino acid sequence of SEQ ID NO:92 (Hu01G06 IGKV1-39 S43A V481);
(z) an immunoglobulin heavy chain variable region comprising the amino acid sequence of SEQ ID NO:54 (Hu01G06 IGHV1-18), and an immunoglobulin light chain variable region comprising the amino acid sequence of SEQ ID NO:92 (Hu01G06 IGKV1-39 S43A V481);
(aa) an immunoglobulin heavy chain variable region comprising the amino acid sequence of SEQ ID NO:56 (Hu01G06 IGHV1-69), and an immunoglobulin light chain variable region comprising the amino acid sequence of SEQ ID NO:92 (Hu01G06 IGKV1-39 S43A V481);
(bb) an immunoglobulin heavy chain variable region comprising the amino acid sequence of SEQ ID NO:58 (Sh01G06 IGHV1-18 M69L), and an immunoglobulin light chain variable region comprising the amino acid sequence of SEQ ID NO:92 (Hu01G06 IGKV1-39 S43A V481);
(cc) an immunoglobulin heavy chain variable region comprising the amino acid sequence of SEQ ID NO:60 (Sh01G06 IGHV1-18 M69L K64Q G44S), and an immunoglobulin light chain variable region comprising the amino acid sequence of SEQ ID NO:92 (Hu01G06 IGKV1-39 S43A V481);
(dd) an immunoglobulin heavy chain variable region comprising the amino acid sequence of SEQ ID NO:62 (Sh01G06 IGHV1-18 M69L K64Q), and an immunoglobulin light chain variable region comprising the amino acid sequence of SEQ ID NO:92 (Hu01G06 IGKV1-39 S43A V481);
(ee) an immunoglobulin heavy chain variable region comprising the amino acid sequence of SEQ ID NO:64 (Sh01G06 IGHV1-69 T30S 169L), and an immunoglobulin light chain variable region comprising the amino acid sequence of SEQ ID NO:92 (Hu01G06 IGKV1-39 S43A V481);
(ff) an immunoglobulin heavy chain variable region comprising the amino acid sequence of SEQ ID NO:66 (Sh01G06 IGHV1-69 T30S K64Q 169L), and an immunoglobulin light chain variable region comprising the amino acid sequence of SEQ ID NO:92 (Hu01G06 IGKV1-39 S43A V481);
(gg) an immunoglobulin heavy chain variable region comprising the amino acid sequence of SEQ ID NO:40 (Ch01G06 Chimeric), and an immunoglobulin light chain variable region comprising the amino acid sequence of SEQ ID NO:94 (Hu01G06 IGKV1-39 V481);
(hh) an immunoglobulin heavy chain variable region comprising the amino acid sequence of SEQ ID NO:54 (Hu01G06 IGHV1-18), and an immunoglobulin light chain variable region comprising the amino acid sequence of SEQ ID NO:94 (Hu01G06 IGKV1-39 V481);
(ii) an immunoglobulin heavy chain variable region comprising the amino acid sequence of SEQ ID NO:56 (Hu01G06 IGHV1-69), and an immunoglobulin light chain variable region comprising the amino acid sequence of SEQ ID NO:94 (Hu01G06 IGKV1-39 V481);
(jj) an immunoglobulin heavy chain variable region comprising the amino acid sequence of SEQ ID NO:58 (Sh01G06 IGHV1-18 M69L), and an immunoglobulin light chain variable region comprising the amino acid sequence of SEQ ID NO:94 (Hu01G06 IGKV1-39 V481);
(kk) an immunoglobulin heavy chain variable region comprising the amino acid sequence of SEQ ID NO:60 (Sh01G06 IGHV1-18 M69L K64Q G44S), and an immunoglobulin light chain variable region comprising the amino acid sequence of SEQ ID NO:94 (Hu01G06 IGKV1-39 V481);
(ll) an immunoglobulin heavy chain variable region comprising the amino acid sequence of SEQ ID NO:62 (Sh01G06 IGHV1-18 M69L K64Q), and an immunoglobulin light chain variable region comprising the amino acid sequence of SEQ ID NO:94 (Hu01G06 IGKV1-39 V481);
(mm) an immunoglobulin heavy chain variable region comprising the amino acid sequence of SEQ ID NO:64 (Sh01G06 IGHV1-69 T30S 169L), and an immunoglobulin light chain variable region comprising the amino acid sequence of SEQ ID NO:94 (Hu01G06 IGKV1-39 V481);
(nn) an immunoglobulin heavy chain variable region comprising the amino acid sequence of SEQ ID NO:66 (Sh01G06 IGHV1-69 T30S K64Q 169L), and an immunoglobulin light chain variable region comprising the amino acid sequence of SEQ ID NO:94 (Hu01G06 IGKV1-39 V481);
(oo) an immunoglobulin heavy chain variable region comprising the amino acid sequence of SEQ ID NO:246 (Hu01G06 IGHV1-18 F1), and an immunoglobulin light chain variable region comprising the amino acid sequence of SEQ ID NO:92 (Hu01G06 IGKV1-39 F1);
(pp) an immunoglobulin heavy chain variable region comprising the amino acid sequence of SEQ ID NO:252 (Hu01G06 IGHV1-69 F2), and an immunoglobulin light chain variable region comprising the amino acid sequence of SEQ ID NO:92 (Hu01G06 IGKV1-39 F1);
(qq) an immunoglobulin heavy chain variable region comprising the amino acid sequence of SEQ ID NO:252 (Hu01G06 IGHV1-69 F2), and an immunoglobulin light chain variable region comprising the amino acid sequence of SEQ ID NO:254 (Hu01G06 IGKV1-39 F2);
(rr) an immunoglobulin heavy chain variable region comprising the amino acid sequence of SEQ ID NO:68 (HE LM 06C11 IGHV2-70), and an immunoglobulin light chain variable region comprising the amino acid sequence of SEQ ID NO:82 (Ch06C11 Chimeric);
(ss) an immunoglobulin heavy chain variable region comprising the amino acid sequence of SEQ ID NO:70 (Hu06C11 IGHV2-5), and an immunoglobulin light chain variable region comprising the amino acid sequence of SEQ ID NO:82 (Ch06C11 Chimeric);
(tt) an immunoglobulin heavy chain variable region comprising the amino acid sequence of SEQ ID NO:46 (Ch06C11 Chimeric), and an immunoglobulin light chain variable region comprising the amino acid sequence of SEQ ID NO:96 (Sh06C11 IGKV1-16);
(uu) an immunoglobulin heavy chain variable region comprising the amino acid sequence of SEQ ID NO:68 (HE LM 06C11 IGHV2-70), and an immunoglobulin light chain variable region comprising the amino acid sequence of SEQ ID NO:96 (Sh06C11 IGKV1-16);
(vv) an immunoglobulin heavy chain variable region comprising the amino acid sequence of SEQ ID NO:70 (Hu06C11 IGHV2-5), and an immunoglobulin light chain variable region comprising the amino acid sequence of SEQ ID NO:96 (Sh06C11 IGKV1-16);
(ww) an immunoglobulin heavy chain variable region comprising the amino acid sequence of SEQ ID NO:72 (Sh14F11 IGHV2-5), and an immunoglobulin light chain variable region comprising the amino acid sequence of SEQ ID NO:86 (Ch14F11 Chimeric);
(xx) an immunoglobulin heavy chain variable region comprising the amino acid sequence of SEQ ID NO:74 (Sh14F11 IGHV2-70), and an immunoglobulin light chain variable region comprising the amino acid sequence of SEQ ID NO:86 (Ch14F11 Chimeric);
(yy) an immunoglobulin heavy chain variable region comprising the amino acid sequence of SEQ ID NO:50 (Ch14F11 Chimeric), and an immunoglobulin light chain variable region comprising the amino acid sequence of SEQ ID NO:98 (Hu14F11 IGKV1-16);
(zz) an immunoglobulin heavy chain variable region comprising the amino acid sequence of SEQ ID NO:72 (Sh14F11 IGHV2-5), and an immunoglobulin light chain variable region comprising the amino acid sequence of SEQ ID NO:98 (Hu14F11 IGKV1-16);
(aaa) an immunoglobulin heavy chain variable region comprising the amino acid sequence of SEQ ID NO:74 (Sh14F11 IGHV2-70), and an immunoglobulin light chain variable region comprising the amino acid sequence of SEQ ID NO:98 (Hu14F11 IGKV1-16); and

An isolated antibody that binds human GDF15 comprising an immunoglobulin heavy chain and an immunoglobulin light chain selected from the group consisting of:

(a) an immunoglobulin heavy chain comprising the amino acid sequence of SEQ ID NO:258 (Hu01G06 IGHV1-18 F2), and an immunoglobulin light chain comprising the amino acid sequence of SEQ ID NO:264 (Hu01G06 IGKV1-39 F2);
(b) an immunoglobulin heavy chain comprising the amino acid sequence of SEQ ID NO:260 (Hu01G06 IGHV1-69 F1), and an immunoglobulin light chain comprising the amino acid sequence of SEQ ID NO:208 (Hu01G06 IGKV1-39 F1);
(c) an immunoglobulin heavy chain comprising the amino acid sequence of SEQ ID NO:176 (Ch01G06 Chimeric), and an immunoglobulin light chain comprising the amino acid sequence of SEQ ID NO:204 (Ch01G06 Chimeric);
(d) an immunoglobulin heavy chain comprising the amino acid sequence of SEQ ID NO:192 (Ch06C11 Chimeric), and an immunoglobulin light chain comprising the amino acid sequence of SEQ ID NO:212 (Ch06C11 Chimeric);
(e) an immunoglobulin heavy chain comprising the amino acid sequence of SEQ ID NO:198 (Ch14F11 Chimeric), and an immunoglobulin light chain comprising the amino acid sequence of SEQ ID NO:216 (Ch14F11 Chimeric);
(f) an immunoglobulin heavy chain comprising the amino acid sequence of SEQ ID NO:178 (Hu01G06 IGHV1-18), and an immunoglobulin light chain comprising the amino acid sequence of SEQ ID NO:206 (Hu01G06 IGKV1-39);
(g) an immunoglobulin heavy chain comprising the amino acid sequence of SEQ ID NO:180 (Hu01G06 IGHV1-69), and an immunoglobulin light chain comprising the amino acid sequence of SEQ ID NO:206 (Hu01G06 IGKV1-39);
(h) an immunoglobulin heavy chain comprising the amino acid sequence of SEQ ID NO:184 (Sh01G06 IGHV1-18 M69L K64Q G44S), and an immunoglobulin light chain comprising the amino acid sequence of SEQ ID NO:210 (Hu01G06 IGKV1-39 V481);
(i) an immunoglobulin heavy chain comprising the amino acid sequence of SEQ ID NO:188 (Sh01G06 IGHV1-69 T30S 169L), and an immunoglobulin light chain comprising the amino acid sequence of SEQ ID NO:210 (Hu01G06 IGKV1-39 V481);
(j) an immunoglobulin heavy chain comprising the amino acid sequence of SEQ ID NO:184 (Sh01G06 IGHV1-18 M69L K64Q G44S), and an immunoglobulin light chain comprising the amino acid sequence of SEQ ID NO:208 (Hu01G06 IGKV1-39 S43A V481);
(k) an immunoglobulin heavy chain comprising the amino acid sequence of SEQ ID NO:188 (Sh01G06 IGHV1-69 T30S 169L), and an immunoglobulin light chain comprising the amino acid sequence of SEQ ID NO:208 (Hu01G06 IGKV1-39 S43A V481);
(1) an immunoglobulin heavy chain comprising the amino acid sequence of SEQ ID NO:256 (Hu01G06 IGHV1-18 F1), and an immunoglobulin light chain comprising the amino acid sequence of SEQ ID NO:208 (Hu01G06 IGKV1-39 F1);
(m) an immunoglobulin heavy chain comprising the amino acid sequence of SEQ ID NO:262 (Hu01G06 IGHV1-69 F2), and an immunoglobulin light chain comprising the amino acid sequence of SEQ ID NO:208 (Hu01G06 IGKV1-39 F1);
(n) an immunoglobulin heavy chain comprising the amino acid sequence of SEQ ID NO:262 (Hu01G06 IGHV1-69 F2), and an immunoglobulin light chain comprising the amino acid sequence of SEQ ID NO:264 (Hu01G06 IGKV1-39 F2);
(o) an immunoglobulin heavy chain comprising the amino acid sequence of SEQ ID NO:194 (HE LM 06C11 IGHV2-70), and an immunoglobulin light chain comprising the amino acid sequence of SEQ ID NO:214 (Sh06C11 IGKV1-16);
(p) an immunoglobulin heavy chain comprising the amino acid sequence of SEQ ID NO:196 (Hu06C11 IGHV2-5), and an immunoglobulin light chain comprising the amino acid sequence of SEQ ID NO:214 (Sh06C11 IGKV1-16);
(q) an immunoglobulin heavy chain comprising the amino acid sequence of SEQ ID NO:200 (Sh14F11 IGHV2-5), and an immunoglobulin light chain comprising the amino acid sequence of SEQ ID NO:218 (Hu14F11 IGKV1-16); and
(r) an immunoglobulin heavy chain comprising the amino acid sequence of SEQ ID NO:202 (Sh14F11 IGHV2-70), and an immunoglobulin light chain comprising the amino acid sequence of SEQ ID NO:218 (Hu14F11 IGKV1-16).

The sequence are located in Table 1A and 2A below.

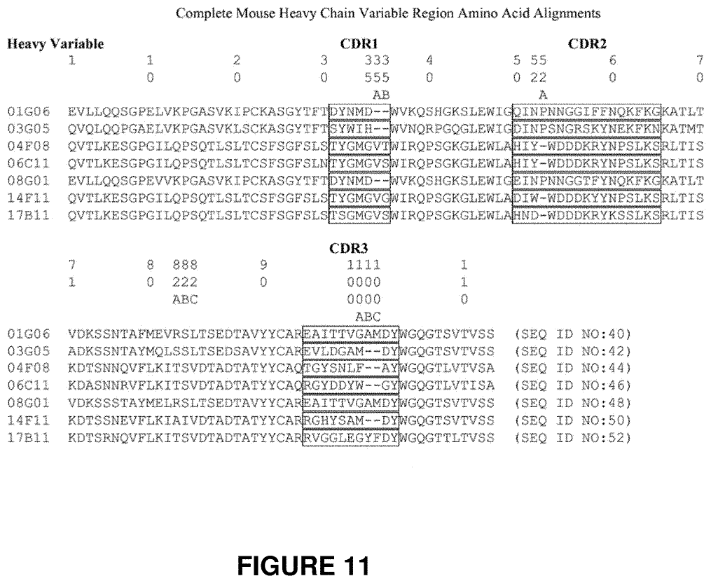

FIG. 11 shows a sequence alignment of the amino acid sequence of the complete immunoglobulin heavy chain variable region of antibodies 01G06, 03G05, 04F08, 06C11, 08G01, 14F11, and 17B11. The amino acid sequences for each antibody are aligned against one another, and CDR1, CDR2, and CDR3, are identified in boxes. The unboxed sequences represent framework (FR) sequences. Alignment positioning (gaps) is based on Kabat numbering, rather than an alignment algorithm such as Clustal. Numbering above the sequences represents Kabat numbering.

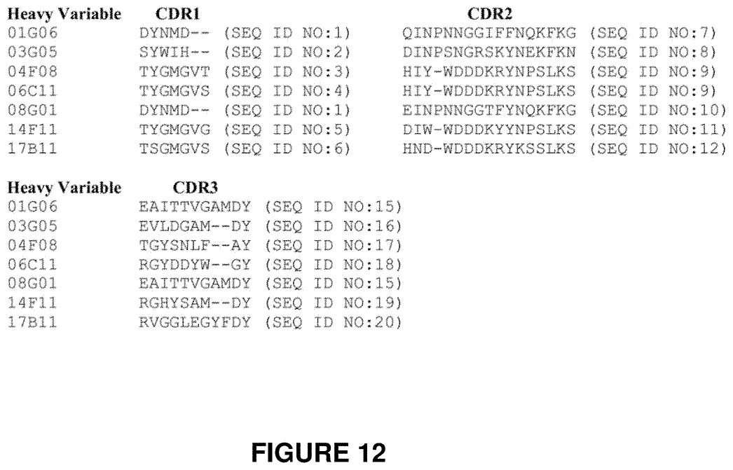

FIG. 12 shows a sequence alignment of the CDR1, CDR2, and CDR3 sequences for each of the immunoglobulin heavy chain variable region sequences in FIG. 11.

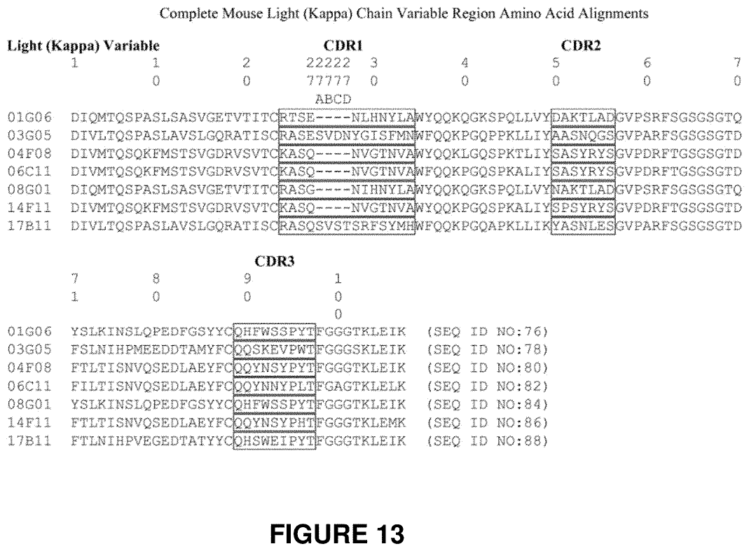

FIG. 13 shows a sequence alignment of the amino acid sequence of the complete immunoglobulin light chain variable region of antibodies 01G06, 03G05, 04F08, 06C11, 08G01, 14F11, and 17B11. The amino acid sequences for each antibody are aligned against one another, and CDR1, CDR2, and CDR3, are identified in boxes. The unboxed sequences represent framework (FR) sequences. Alignment positioning (gaps) is based on Kabat numbering, rather than an alignment algorithm such as Clustal. Numbering above the sequences represents Kabat numbering.

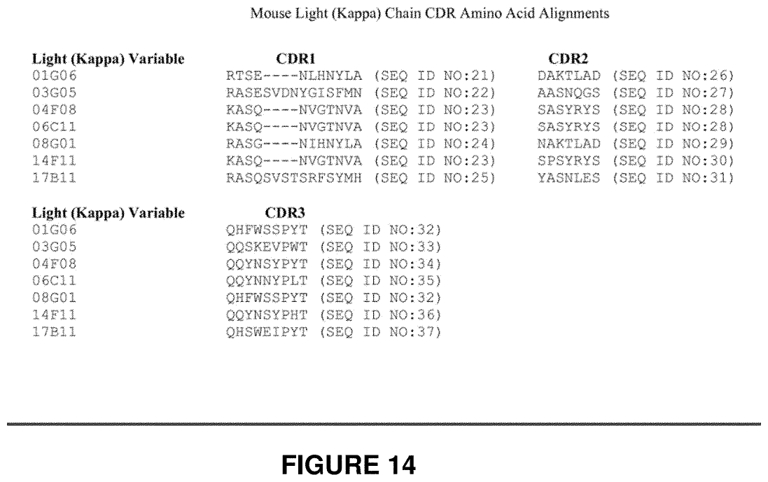

FIG. 14 shows a sequence alignment of the CDR1, CDR2, and CDR3 sequences for each of the immunoglobulin light chain variable region sequences in FIG. 13.

TABLE 1A

| | | | |
|---|---|---|---|
| | | | Variable |
| | | | Region |
| | | | SEQ ID |
| CDR1 | CDR2 | CDR3 | NO: |

Kabat

| | | | | |
|---|---|---|---|---|
| Ch01G06 | DYNMD | QINPNNGGIFFNQKFKG | EAITTVGAMDY | 40 |
| Chimeric | (SEQ ID NO: 1) | (SEQ ID NO: 7) | (SEQ ID NO: 15) | |
| Hu01G06 | DYNMD | QINPNNGGIFFNQKFKG | EAITTVGAMDY | 54 |
| IGKV1-18 | (SEQ ID NO: 1) | (SEQ ID NO: 7) | (SEQ ID NO: 15) | |
| Hu01G06 | DYNMD | QINPNNGGIFFNQKFKG | EAITTVGAMDY | 56 |
| IGKV1-69 | (SEQ ID NO: 1) | (SEQ ID NO: 7) | (SEQ ID NO: 15) | |
| Sh01G06 | DYNMD | QINPNNGGIFFNQKFKG | EAITTVGAMDY | 58 |
| IGHV1-18 M69L | (SEQ ID NO: 1) | (SEQ ID NO: 7) | (SEQ ID NO: 15) | |
| Sh01G06 IGHV1-18 | DYNMD | QINPNNGGIFFNQKFQG | EAITTVGAMDY | 60 |
| M69L K64Q | (SEQ ID NO: 1) | (SEQ ID NO: 13) | (SEQ ID NO: 15) | |
| G44S | | | | |
| Sh01G06 IGHV1-18 | DYNMD | QINPNNGGIFFNQKFQG | EAITTVGAMDY | 62 |
| M69L K64Q | (SEQ ID NO: 1) | (SEQ ID NO: 13) | (SEQ ID NO: 15) | |
| Sh01G06 IGHV1-69 | DYNMD | QINPNNGGIFFNQKFKG | EAITTVGAMDY | 64 |
| T30S I69L | (SEQ ID NO: 1) | (SEQ ID NO: 7) | (SEQ ID NO: 15) | |
| Sh01G06 IGHV1-69 | DYNMD | QINPNNGGIFFNQKFQG | EAITTVGAMDY | 66 |
| T30S K64Q | (SEQ ID NO: 1) | (SEQ ID NO: 13) | (SEQ ID NO: 15) | |
| I69L | | | | |
| Hu01G06 | DYNMD | QINPYNHLIFFNQKFQG | EAITTVGAMDY | 246 |
| IGKV1-18 F1 | (SEQ ID NO: 1) | (SEQ ID NO: 236) | (SEQ ID NO: 15) | |
| Hu01G06 | DYNMD | GINPNNGLIFFNQKFQG | EAITTVGAMDY | 248 |
| IGKV1-18 F2 | (SEQ ID NO: 1) | (SEQ ID NO: 237) | (SEQ ID NO: 15) | |
| Hu01G06 | DYNMD | GINPNNGLIFFNQKFKG | EAITTVGAMDY | 250 |
| IGKV1-69 F1 | (SEQ ID NO: 1) | (SEQ ID NO: 238) | (SEQ ID NO: 15) | |
| Hu01G06 | DYNMD | QINPYNHLIFFNQKFKG | EAITTVGAMDY | 252 |
| IGHV1-69 F2 | (SEQ ID NO: 1) | (SEQ ID NO: 235) | (SEQ ID NO: 15) | |
| Ch01G06 | TYGMGVS | HIYWDDDKRYNPSLKS | RGYDDYWGY | 46 |
| Chimeric | (SEQ ID NO: 4) | (SEQ ID NO: 9) | (SEQ ID NO: 18) | |
| HE LM 06C11 | TYGMGVS | HIYWDDDKRYNPSLKT | RGYDDYWGY | 68 |
| IGHV1-70 | (SEQ ID NO: 4) | (SEQ ID NO: 14) | (SEQ ID NO: 18) | |
| Hu01G06 | TYGMGVS | HIYWDDDKRYNPSLKS | RGYDDYWGY | 70 |
| IGHV2-5 | (SEQ ID NO: 4) | (SEQ ID NO: 9) | (SEQ ID NO: 18) | |
| Ch14F11 | TYGMGVG | DIWWDDDKYYNPSLKS | RGHYSAMDY | 50 |
| Chimeric | (SEQ ID NO: 5) | (SEQ ID NO 11) | (SEQ ID NO 19) | |
| Sh14F11 | TYGMGVG | DIWWDDDKYYNPSLKS | RGHYSAMDY | 72 |
| IGHV2-5 | (SEQ ID NO: 5) | (SEQ ID NO: 11) | (SEQ ID NO: 19) | |
| Sh14F11 | TYGMGVG | DIWWDDDKYYNPSLKS | RGHYSAMDY | 74 |
| IGHV2-70 | (SEQ ID NO: 5) | (SEQ ID NO: 11) | (SEQ ID NO: 5) | |

Chothia

| | | | | |
|---|---|---|---|---|
| Ch01G06 | GYTFTDY | NPNNGG | EAITTVGAMDY | 40 |
| Chimeric | (SEQ ID NO: 38) | (SEQ ID NO: 143) | (SEQ ID NO: 15) | |
| Hu01G06 | GYTFTDY | HPHUGC | EAITTVGAMDY | 54 |
| IGHV1-18 | (SEQ ID NO: 38) | (SEQ ID NO: 143) | (SEQ ID NO: 15) | |
| Hu01G06 | GYTFTDY | NPNNGG | EAITTVGAMDY | 56 |
| IGHV1-69 | (SEQ ID NO: 38) | (SEQ ID NO: 143) | (SEQ ID NO: 15) | |
| Sh01G06 | GYTFTDY | NPNNGG | EAITTVGAMDY | 55 |
| IGHV1-18 M69L | (SEQ ID NO: 38) | (SEQ ID NO: 143) | (SEQ ID NO: 15) | |
| Sh01G06 | GYTFTDY | NPNNGG | EAITTVGAMDY | 60 |
| IGHV1-18 | (SEQ ID NO: 38) | (SEQ ID NO: 143) | (SEQ ID NO: 15) | |
| M69L K64Q | | | | |
| G44S | | | | |
| Sh01G06 IGHV1- | GYTFTDY | NPNNGG | EAITTVGAMDY | 62 |
| 18 M69L K64Q | (SEQ ID NO: 38) | (SEQ ID NO: 143) | (SEQ ID NO: 15) | |
| Sh01G06 | GYTFSDY | NPNNGG | EAITTVGAMDY | 64 |
| IGHV1-69 | (SEQ ID NO: | (SEQ ID NO: 143) | {SEQ ID NO: 15) | |
| T30S I69L | 234) | | | |
| Sh01G06 IGHV1- | GYTFSDY | NPNNGG | EAITTVGAMDY | 66 |
| 69 T30S K64Q | (SEQ ID NO: | (SEQ ID NO: 143) | (SEQ ID NO: 15) | |
| I69L | 234) | | | |
| Hu01G06 | GYTFTDY | NPYNHL | EAITTVGAMDY | 246 |
| IGHV1-18 F1 | (SEQ IS NO: 38) | (SEQ ID NO: 240) | (SEQ ID NO: 15) | |
| Hu01G06 | GYTFTDY | NPNNGL | EAITTVGAMDY | 248 |
| IGHV1-18 F2 | (SEQ ID NO: 38) | (SEQ ID NO: 241) | (SEQ ID NO: 15) | |
| Hu01G06 | GYTFSDY | NPNNGL | EAITTVGAMDY | 250 |
| IGHV1-69 F1 | (SEQ ID NO: | (SEQ ID NO: 241) | (SEQ ID NO: 15) | |
| | 234) | | | |
| Hu01G06 | GYTFSDY | NPYNHL | EAITTVGAMDY | 252 |
| IGHV1-69 F2 | (SEQ ID NO: | (SEQ ID NO: 240) | (SEQ ID NO: 15) | |
| | 234) | | | |
| Ch01G06 | GFSLNTYGM | YWDDD | RGYDDYWGY | 46 |
| Chimeric | (SEQ ID NO: | (SEQ ID NO: 145) | (SEQ ID NO: 18) | |
| | 132) | | | |
| HE LM 06C11 | GFSLNTYGM | YWDDD | RGYDDYWGY | 68 |
| IGHV2-70 | (SEQ ID NO: | (SEQ ID NO: 145) | (SEQ ID NO: 18) | |
| | 132) | | | |
| Hu01G06 | GFSLNTYGM | YWDDD | RGYDDYWGY | 70 |
| IGHV2-5 | (SEQ ID NO: | (SEQ ID NO: | | |
| (SEQ ID | 145) | 18) | | |
| NO: 132) | | | | |
| Ch14F11 | GFSLSTYGM | WWDDD | RGHYSAMDY | 50 |
| Chimeric | (SEQ ID NO: 146) | (SEQ ID NO: 19) | | |
| (SEQ ID | | | | |
| NO: 130) | | | | |
| Sh14F11 | GFSLSTYGM | WWDDD | RGHYSAMDY | 72 |
| IGHV2-5 | (SEQ ID NO: 146) | (SEQ ID NO: 19) | | |
| (SEQ ID | | | | |
| NO: 130) | | | | |
| Sh14F11 | GFSLSTYGM | WWDDD | RGHYSAMDY | 74 |
| IGHV2-70 | (SEQ ID NO: 130) | (SEQ ID NO: 146) | (SEQ ID NO: 19) | |

IMGT

| | | | | |
|---|---|---|---|---|
| Ch01G06 | GYTFTDYN | INPNNGGI | AREAITTVGAMDY | 40 |
| Chimeric | (SEQ ID NO: | (SEQ ID NO: 148) | (SEQ ID NO: 154) | |
| | 136) | | | |
| Hu01G06 | GYTFTDYN | INPNNGGI | AREAITTVGAMDY | 54 |
| IGHV1-I8 | (SEQ ID NO: | (SEQ ID NO: 148) | (SEQ ID NO: 154) | |
| | 136) | | | |
| Hu01G06 | GYTFTDYN | INPNNGGI | AREAITTVGAMDY | 56 |
| IGHV1-69 | (SEQ ID NO: | (SEQ ID NO: 148) | (SEQ ID NO: 154) | |
| | 136) | | | |
| Sh01G06 | GYTFTDYN | INPNNGGI | AREAITTVGAMDY | 58 |
| IGHV1-18 M69L | (SEQ ID NO: | (SEQ ID NO: 148) | (SEQ ID NO: 154) | |
| | 136) | | | |
| Sh01G06 | GYTFTDYN | INPNNGGI | AREAITTVGAMDY | 60 |
| IGHV1-18 M69L | (SEQ ID NO: | (SEQ ID NO: 148) | (SEQ ID NO: 154) | |
| K64Q G44S | 136) | | | |
| Sh01G06 | GYTFTDYN | INPNNGGI | AREAITTVGAMDY | 62 |
| IGHV1-18 M69L | (SEQ ID NO: | (SEQ ID NO: 148) | (SEQ ID NO: 154) | |
| K64Q | 136) | | | |
| Sh01G06 | GYTFSDYN | INPNNGGI | AREAITTVGAMDY | 64 |
| IGHV1-18 T30S | (SEQ ID NO: | (SEQ ID NO: 148) | (SEQ ID NO: 154) | |
| I69L | 235) | | | |
| Sh01G06 | GYTFSDYN | INPNNGGI | AREAITTVGAMDY | 66 |
| IGHVI-69 T30S | (SEQ ID NO: | (SEQ ID NO: 148) | (SEQ ID NO: 154) | |
| | 235) | | | |
| K64Q I69L | | | | |
| Hu01G06 | GYTFTDYN | INPYNHLI | AREAITTVGAMDY | 246 |
| IGHV1-18 F1 | (SEQ ID NO: | (SEQ ID NO: 242) | (SEQ ID NO: 154) | |
| | 136) | | | |
| Hu01G06 | GYTFTDYN | INPNNGLI | AREAITTVGAMDY | 248 |
| IGHV1-18 F2 | (SEQ ID NO: | (SEQ ID NO: 243) | (SEQ ID NO: 154) | |
| | 138) | | | |
| Hu01G06 | GYTFSDYN | INPNNGLI | AREAITTVGAMDY | 250 |
| IGHV1-69 F1 | (SEQ ID NO: | (SEQ ID NO: 243) | (SEQ ID NO: 154) | |
| | 235) | | | |
| Hu01G06 | GYTFSDYN | INPYNHLI | AREAITTVGAMDY | 252 |
| IGHV1-69 F2 | (SEQ ID NO: | (SEQ ID NO: 242) | (SEQ ID NO: 154) | |
| | 235) | | | |
| Ch06C11 | GFSLNTYGMG | IYWDDDK | AQRGYDDYWGY | 46 |
| Chimeric | (SEQ ID NO. | (SEQ ID NO: 150) | (SEQ ID NO: 157) | |
| | 141) | | | |
| HE LM 06C11 | GFSLNTYGMG | IYWDDDK | AQRGYDDYWGY | 68 |
| IGHV2-70 | (SEQ ID NO: | (SEQ ID NO: 150) | (SEQ ID NO: 157) | |
| | 141) | | | |
| Hu01G06 | GFSLNTYGMG | IYWDDDK | AQRGYDDYWGY | 70 |
| IGHV2-5 | (SEQ ID NO. | (SEQ ID NO: 150) | (SEQ ID NO: 157) | |
| | 141) | | | |
| Ch14F11 | GFSLSTYGMG | IWWDDDK | ARRGHYSAMDY | 50 |
| Chimeric | (SEQ ID NO: | (SEQ ID NO: 152) | (SEQ ID NO: 158) | |
| | 140) | | | |
| Sh14F11 | GFSLSTYGMG | IWWDDDK | | 72 |
| IGHV2-5 | (SEQ ID NO: | (SEQ ID NO: 158) | | |
| (SEQ ID | 152) | | | |
| NO: 140) | | | | |
| Sh14F11 | GFSLSTYGMG | IWWDDDK | ARRGHYSAMDY | 74 |
| IGHV2-70 | (SEQ ID NO: | (SEQ ID NO: 152) | (SEQ ID NO: 158) | |
| | 140) | | | |

TABLE 2A

| | | | |
|---|---|---|---|
| | | | Variable |
| | | | Region |
| | | | SEQ |
| CDR1 | CDR2 | CDR3 | ID NO: |

Kabat/Chothia

| | | | | |
|---|---|---|---|---|
| Ch01G06 | RTSENLHNYLA | DAKTLAD | GHFWSSFYT | 76 |
| Chimeric | (SEQ ID NO: 21) | (SEQ ID NO: 28) | (SEQ ID NO: 32) | |
| Hu01G06 | RTSENLHNYLA | DAKTLAD | GHFWSSFYT | 90 |
| IGKV1-39 | (SEQ ID NO: 21) | (SEQ ID NO: 28) | (SEQ ID NO: 32) | |
| Hu01G06 | RTSENLHNYLA | DAKTLAD | GHFWSSFYT | 92 |
| IGKV1-38 | (SEQ ID NO: 21) | (SEQ ID NO: 28) | (SEQ ID NO: 32) | |
| S43A V48I | | | | |
| (also known | | | | |
| as Hu01G06 | | | | |
| IGKV1-39 F1) | | | | |
| Hu01G06 | RTSENLHNYLA | DAKTLAD | GHFWSSFYT | 94 |
| IGKV1-38 | (SEQ ID NO: 21) | (SEQ ID NO: 28) | (SEQ ID NO: 32) | |
| V48I | | | | |
| Hu01G06 | RTSENLHNYLA | DAKTLAD | GHFWSSFYT | 92 |
| IGKV1-39 F1 | (SEQ ID NO: 21) | (SEQ ID NO: 28) | (SEQ ID NO: 32) | |
| (also known | | | | |
| as Hu01G06 | | | | |
| IGKV1-39 | | | | |
| S43A V48I) | | | | |
| Hu01G06 | RTSENLHNYLA | DAKTLAD | GHFWSDPYT | 254 |
| IGKV1-38 F2 | (SEQ ID NO: 21) | (SEQ ID NO: 28) | (SEQ ID NO: 244) | |
| Ch06C11 | KASQNVGTNVA | SASYRYS | QQYNNYPLT | 82 |
| Chimeric | (SEQ ID NO: 23) | (SEQ ID NO: 28) | (SEQ IO NO: 35) | |
| Sh06C11 | KASQNVGTNVA | SASYRYS | QQYNNYPLT | 96 |
| IGKV1-16 | (SEQ ID NO: 23) | (SEQ ID NO: 28) | (SEQ ID NO: 35) | |
| Ch14F11 | KASQNVGTNVA | SPSYRYS | QQYNSYPHT | 86 |
| Chimeric | (SEQ ID NO: 23) | (SEQ ID NO: 30) | (SEQ ID NO: 36) | |
| Hu14F11 | KASQNVGTNVA | SPSYRYS | QQYNSYPHT | 98 |
| IGKV1-16 | (SEQ ID NO: 23) | (SEQ ID NO: 30) | (SEQ ID NO: 36) | |

IMGT

| | | | | |
|---|---|---|---|---|
| Ch01G06 | ENLHNY | DAK | QHFWSSFYT | 78 |
| Chimeric | (SEQ ID NO: 160) | | (SEQ ID NO: 32) | |
| Hu01G06 | ENLHNY | DAK | QHFWSSFYT | 90 |
| IGKV1-39 | (SEQ ID NO: 160) | | (SEQ ID NO: 32) | |
| Hu01G06 | ENLHNY | DAK | QHFWSSFYT | 92 |
| IGKV1-39 | (SEQ ID NO: 160) | | | |
| S43A V48I | | | | |
| (also | | | | |
| known as | | | | |
| Hu01G06 | | | | |
| IGKV1-39 F1) | | | | |
| Hu01G06 | ENLHNY | DAK | QHFWSSFYT | 94 |
| IGKV1-39 | (SEQ ID NO: 160) | | | |
| V48I | | | | |
| Hu01G06 | ENLHNY | DAK | QHFWSSPYT | 92 |
| IGKV1-39 | (SEQ ID NO: 160) | | (SEQ ID NO: 32) | |
| F1 (also | | | | |
| known as | | | | |
| Hu01G06 | | | | |
| IGKV1-39 | | | | |
| S43A V48I) | | | | |
| Hu01G06 | ENLHNY | DAK | QHFWSDPYT | 254 |
| IGKV1-39 F2 | (SEQ ID NO: 160) | | (SEQ ID NO: 244) | |
| Ch06c11 | QNVGTN | SAS | QQYNNYPLT | 82 |
| Chimeric | (SEQ ID NO: 162) | | (SEQ ID NO: 35) | |
| Sh06c11 | QNVSTN | SAS | QQYNNYPLT | 96 |
| IGKV1-16 | (SEQ ID NO: 132) | | (SEQ ID NO: 35) | |
| Ch14F11 | QNVSTN | SPS | QQYNSYPHT | |
| Chimeric | (SEQ ID NO: 182) | | (SEQ ID NO: 36) | 86 |
| Hu14F11 | QNVSTN | SPS | QQYNSYPHT | 93 |
| IGKV1-16 | (SEQ ID NO: 182) | | (SEQ ID NO: 36) | |

### Example 2 Methods for Manufacturing Particles

Method for Manufacturing Silicon Particles

Porous silicon disks could be produced with sizes of 997 nm by 398 nm and 998 nm by 798 nm with variable pore sizes. The structure and form of these discs could be characterized by scanning electron microscopy. Gold nanoparticles could be deposited in the pores of the porous silicon disks. Ligand could be attached to the surfaces of the gold nanoparticles through covalent bonds.

Method for Manufacturing Polymer Particles

The production of particles from Poly(lactide-co-glycolide) (PLGA) particles could be done by the following procedure. The control of the size, morphology, coating density and uniformity of the PLGA particles could be done by scanning electron microscopy, atomic force microscopy, and transmission electron microscopy.

The particles should be coated with quaternary ammonium beta-cyclodextrin. The beta-cyclodextrin-coated PLGA particles could be coated with a blend of polyethylene glycol (PEG) and thiol moieties to allow. The stability of the coating could be characterized by incubating the particles in media for various periods of time. During the incubation with macrophages at the different time points, the evasion and uptake of the particles could be studied. The PLGA particles could be coated with Ligand. The superparticle (as the combination of several particles) could be combined by disulfide bonds. The exterior surface (i.e., outer surface) of the superparticle could be optionally blocked with particles that do not comprise Ligand to prevent interactions between the Ligand of the superparticle and cells.

### Example 3 Evaluation of Binder Selectivity

As a rule the characterization and development of highly specific binders requires the analysis of the interaction strength between binder and target. For analysis the MicroScale Thermophoresis (MST) can be used to quantify biomolecular interactions in solution at microliter scale.

The selectivity of the binders could be evaluated by comparison Kd in MST experiment in buffer and plasma. In case of unspecific interaction the binder with components of plasma, the Kd value significantly reduces. Quantitative comparison of the buffer-dependence of the Kd of the target-aptamer interaction gives estimation of the selectivity, which could be calculated using the formula (1)

Selectivity=Kd in buffer/Kd in plasma (1)

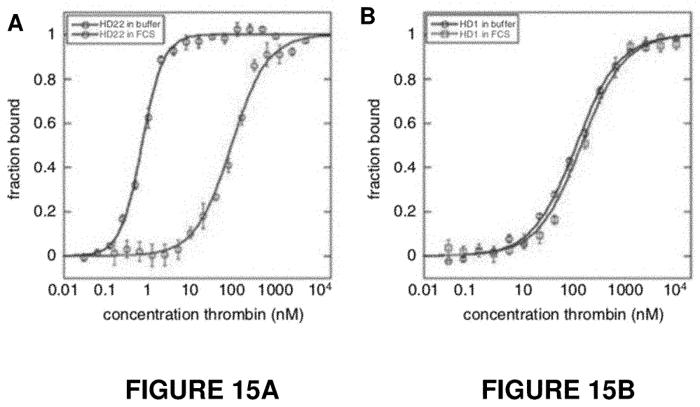

There is an example of the selective and unselective interaction, see FIGS. 15A and 15B (the figures are taken from Chapter 8 “Aptamer Binding Studies Using MicroScale Thermophoresis” of the GOnter Mayer (ed.), Nucleic Acid Aptamers: Selection, Characterization, and Application, Methods in Molecular Biology, vol. 1380, DOI 10.1007/978-1-4939-3197-2_8, © Springer Science+Business Media New York 2016).

FIG. 15A is a fraction bound plot of the MST experiments HD22 aptamer binding to thrombin in buffer or serum and FIG. 15B is a fraction bound plot of the MST experiments HD1 aptamer binding to thrombin in buffer or serum. A quantitative comparison of the buffer-dependence of the Kd of the thrombin-aptamer interaction shows that the serum has a considerable influence on the binding of HD22 to thrombin. The affinity decreases from 0.71 nM to 90.5 nM.

### Example 4 Immunization (Polyclonal Antibody Production)

Polyclonal antibodies can be produced by immunization of animals (e.g., rat, mouse, rabbit, sheep) with the protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB or peptide fragment thereof (antigen) by methods well-known to those skilled in the art [Antibodies: A laboratory Manual, ed. by Lane, H, D. et al., Cold Spring Harbor Laboratory Press, New York, 1989]. Briefly, 2.5 mg of for example recombinant full length mature BMP4 (Ser293-Arg408, ENSG00000125378) protein (for example, Cat No 314-BP-01M, R&D Systems) or any other Target is required to immunize two rabbits. The concentration should be >0.4 mg/ml, and the antigen solution must not contain organic solvents. The antigen/adjuvant mixture is inoculated multiple site subcutaneously along the back and intramuscularly in the hind limbs (0.1 ml per site). 1.0 ml of antigen is mixed with 1.0 ml of for initial inoculation and 1.0 ml of for the following injections (this makes 2.0 ml of mixture for 10 subcutaneous sites and 10 intramuscular sites). The schedule of immunization can be as follows:

Day 0 Pre-immune bleed (5 mL per rabbit)
Day 1 Initial immunization (0.5 mg of immunogen in Complete Freund's Adjuvant), 10 SQ sites
Day 14 Boost with 0.25 mg of immunogen in Incomplete Freund's Adjuvant, 4SQ sites

### Day 28 Boost (as on Day 14)

Day 35 Bleed (˜25 mL per rabbit)

### Day 42 Boost (as on Day 14)

Day 56, 70 Production bleeds (˜25 mL per rabbit)
Antibody is then antigen-affinity purified.
In rare case if the procedure did not give the positive results it can be repeated with or without reasonable changes with other animals and reactives obtained from other sources as expert in the field can trivially decide.
Great variety of polyclonal antibodies are available for purchase and contract manufacturing, some of which are described in this disclosure.

### Example 5 Immobilization of Antibodies

Antibodies (or other proteins) binding protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB can be immobilized on many stationary phases in many ways known in the art, for example it can be immobilized on a cyanogen bromide-activated Sepharose for subsequent use in immunosorbent (or affinity) apheresis or sorption column for example as shown in [Kavran J M, Leahy D J. Coupling antibody to cyanogen bromide-activated sepharose. Methods Enzymol. 2014; 541:27-34]. Cyanogen bromide (CNBr)-activated Sepharose is a readily available commercial product (e.g. #17043001, GE Healthcare). Proteins are coupled to the resin through primary amines. CNBr-based resin is a good choice because of the mild reaction conditions and broad applicability to different types of proteins.

One of the examples could be coupling sFRP-3 (FRZB) antibody to the sorbent All buffers planned to be used should be sterile filtered. 500 mg of Human/Mouse sFRP-3 (FRZB) Antibody (Cat #MAB1921, R&D Systems, USA) is dialyzed into cold Coupling Buffer (0.1 M NaHCO₃, 0.5 M NaCl, pH 9.0) at 4° C. for 2 h, then the buffer is changed for new one and dialysis is continued for another 2 h. The final concentration of the antibody should be 1-2 mg/ml, if lower antibody should be concentrated. (CNBr)-activated Sepharose beads are left to swell in 1 mM HCl for 15 min. at room temp (0.25 g of dry resin gives 1 ml of swollen resin), the gel is transferred to sintered glass funnel and washed with 1 mM HCl and then with coupling buffer. This is immediately resuspended in a solution of the antibody (2 mg of antibody per 1 ml of swollen resin) and incubated on a nutator or platform rocker for 2 h at 4° C. This is then centrifuged, the supernatant is decanted and sediment is resuspended in 1 M Tris-Base, pH 9.0 in coupling buffer to block the remaining unreacted CNBr sites and incubated for 2 h at room temperature. This is then washed thoroughly with 20 volumes of PBS.

### Example 6. Recombinant Protein Production

Recombinant proteins can be as well used as binders and medications binding or targeting target proteins described in this application. Recombinant proteins are produced by methods known to those skilled in the art [e.g. but not limited to as described Tools and Techniques in Biomolecular Science. Chapter 7: Recombinant Protein Expression, ed by Divan A, Royds J, Oxford University Press, 2013] pp& 145-175. For example, for production of recombinant myostatin protein UniProt ID 014793, that can be used as a binder of FSTL3, the sequence corresponding to the mature protein Asp268-Ser376 is cloned into mammalian cell expressing vector with His-tag, e.g., pcDNA3.1/HisA,B,C (ThermoScientific, USA). Competent E. coli strain (e.g., TOP10, DH5a) is transformed with ligation mixture of the Target sequence and the vector, positive clones are selected on LB plates containing 100 mg/mL ampicillin. Selected colonies (10-20 colonies) are analyzed for the presence and orientation of the insert. Then the cloned plasmid is harvested and sequenced. After the sequence is confirmed the plasmid is transfected into HEK293 cell line using Lipofectamine reagent (ThermoScientific, USA). For stable transfection selective antibiotic (Geneticin, ThermoScientific, USA) is added to culture medium. HEK293 cells are harvested for protein preparation and lysed. Nickel-chelating resin can be used for His-tag-fused protein isolation, e.g ProBond Purification System (ThermoScientific, USA), according to manufacturer's protocol.

### Example 7 Production of the Column

### Manufacturing and Tests

### The Housing of Column

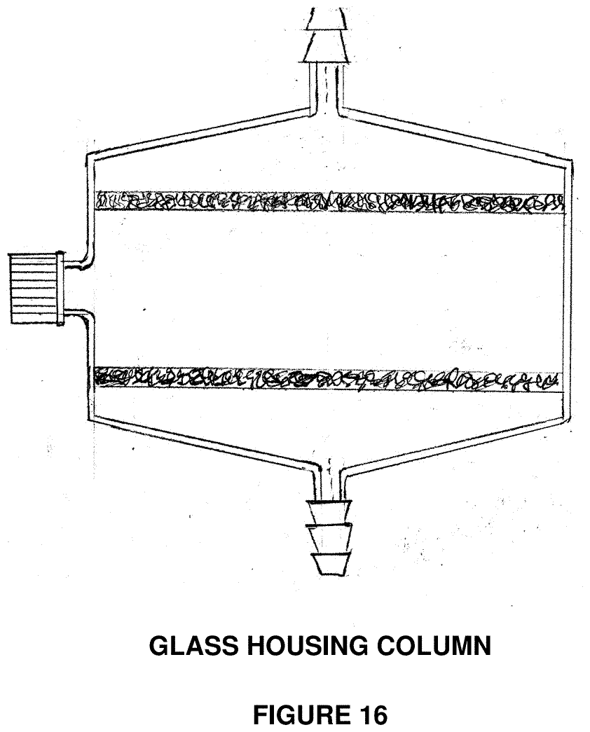

The borosilicate glass 3.3 (ISO 3585) could be used for producing the glass housing of apheresis column (in preferred case it should be symmetrical (see FIG. 16); the column on each side could have glass filter plates with porosity 16-40 μm. There could be different types of housing volumes: from 100 to 500 ml.

(i) Active Ingredient

The active ingredient of column could be synthetic binder covalently bound with stationary phase-agarose beads:
1) The 5′-amino C6 modified DNA aptamer 5′-NH2-C6-ACGAGYCYGGYCCYYCCYCCCYAAYYGCYGYYGAGGYAYCGGCYACAACA A-3′ against POSTN
2) The Human/Mouse sFRP-3 (FRZB) Antibody (Cat #MAB1921, R&D Systems, USA) against sFRP-3
3) The 3-{N-[(3S,5R)-5-(hydroxycarbamoyl)-1-(4-methoxybenzenesulfonyl)pyrrolidin-3-yl]methanesulfonamido}propyl group against MMP-7

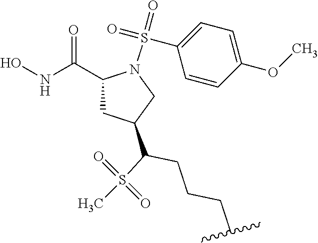 [SMILES: *CCCC([C@H]1C[C@H](C(=O)NO)N(S(=O)(=O)c2ccc(OC)cc2)C1)S(C)(=O)=O]

Mix of equal parts of agarose beads with different binders (100 g of agarose beads with immobilized the 5′-amino C6 modified DNA aptamer against POSTN plus 100 g of agarose beads with immobilized the Human/Mouse sFRP-3 (FRZB) Antibody (Cat #MAB1921, R&D Systems, USA) against sFRP-3 and 100 g of with agarose beads with immobilized the 3-{N-[(3S,5R)-5-(hydroxycarbamoyl)-1-(4-methoxybenzenesulfonyl)pyrrolidin-3-yl]methanesulfonamido}propyl group against MMP-7) could be handled as single adsorption gel.

Adsorption gel will be insoluble in water buffers, plasma and other hydrophilic solutions. It will have no pharmacological function itself and won't release any substances into the body. The gel beads could have an average diameter between 45 and 165 m.

The covalent chemical bond between binders and matrix will be very strong. Chemical and toxicological evaluation of extracts should reveal no considerable leakages of any chemical and/or toxic substances.

(ii) Assembled Column

Adsorption gel could be suspended in buffered physiological solution and filled into glass housing.

Adsorption gel could be closed inside the column between two glass filters. Column could be assembled with plastic lines for the connection with primary device during the application.

The assembling could be made during the production process. The lines could be hermetically closed by closed screw caps. Caps will have the special labels “unauthorized open protection” which prevent the accidental opening of column before the use. This label should be removed after first use. Caps should be closed during columns transportation, before and between columns application.

(iii) Manufacturing

Manufacture of columns should be conducted in the rooms for production the medical devices. All personnel engaged in production and quality control should experienced the appropriate training and attestation.

The production process will include the following main stages:

I. Preparation of the sorbent.

II. Filling the columns.

III. Sterilization of columns.

IV. Marking and packing of columns.

V. Production of regenerating solutions.

All processes of the production and quality control should be described in standard operating procedures. Parameters of the raw materials, intermediate products, and final product should be given in the relevant specifications. The production facilities in which production processes are carried out should be controlled.

Stage I. Obtaining the Sorbent.

For the synthesis of the sorbent, the agarose matrix Sepharose, GE Healthcare, USA could be used. Similar matrices are used, for example, for the synthesis of sorbents for extracorporal therapy (LDL Lipopak®, Lp (a) Lipopak®, Ig Adsopak®, ABO Adsopak® manufactured by POCARD Ltd. 121552, 3-rd Cherepkovskaya Street 15A, Moscow, Russia http://www.pocard.ru/). Matrix Sepharose, like other matrices used for the synthesis of sorbents for medical purposes, is inert (does not interact with blood components) and hemocompatible. Immobilization of the ligand should be carried out to obtain a specific for a particular component of the blood sorbent. The heparin, Bioiberica, Spain could be used for the preparation of the column. This substance is used in the pharmaceutical industry for the manufacture of pharmaceuticals, and it is not toxic.

The immobilization process consists of the steps described in the corresponding section of this disclosure. The unbound ligand should be removed by washing after completion of the immobilization. The final product could be the suspension of gel beads in water. This material will be chemically stable, and it withstands the autoclaving procedure.

Stage II. Filling the Columns.

The column could be filled with mix of the sorbents suspension (1 part of agarose beads with immobilized the 5′-amino C6 modified DNA aptamer against POSTN plus 1 part of agarose beads with immobilized the Human/Mouse sFRP-3 (FRZB) Antibody (Cat #MAB1921, R&D Systems, USA) against sFRP-3 and 1 part of with agarose beads with immobilized the 3-{N-[(3S,5R)-5-(hydroxycarbamoyl)-1-(4-methoxybenzenesulfonyl)pyrrolidin-3-yl]methanesulfonamido}propyl group against MMP-7). The filling process could be carried out manually, without any equipment. 250+/−10 ml of sorbent should be placed in each column, the free space of the column should be filled with water. The process should be carried out in a laminar box. Each series of sorbent should be tested for microbiological purity before filling it in a column. After filling the column will label with heat-resistant self-adhesive labels with the name of the column, volume, storage conditions, etc. and place in individual Steriking packages (Wihuri OY Wipak, Finland) for autoclaving. Individual packages with columns could be marked with heat-resistant self-adhesive labels with the name of the column, volume, storage conditions, etc.

Stage III. Sterilization of Columns.

Sterilization of the columns should be carried out by autoclaving in a steam sterilizer Sanyo MLS-3781L for 35 minutes at the temperature of 121C. The sterilizer undergoes an annual qualification procedure, the purpose of which will be to confirm the homogeneity of temperature distribution in the sterilizer chamber. Parameters of the autoclaving process of the columns should be validated. To test the sterility of each column series, the sterility of washings from the columns (the number is 0.4*sqrt (n), where n is the number of columns in the series) could be provided.

Stage IV. Packing and Marking.

Each column should be controlled by visual inspection during which the tightness and appearance of the column are checked. The package with a column could be placed in an individual case made of corrugated plastic with foam polyethylene lodgment.

### Example 8 Testing the Stability and Reproducibility of the Column Against 3 Targets (POSTN, sFRP-3 and MMP-7)

Stability and Reproducibility could be tested with human plasma by the measurement the change in the retention and elution behavior each of 3 proteins (POSTN, sFRP-3 and MMP-7) after the repeated use of the column at 20° C. every day for one month.

The retention time of target eluted by 2 M NaClO4 could be measured, the relative standard deviation of this parameter should be as small as possible, preferable embodiment not more significant than 40%, measured over one month.

The column not in use could be filled with the binding buffer solution and stored at 5° C.

The reproducibility can be studied in the triplicate analyses of a sample on the same column. The obtained relative standard deviation in the peak area of the each eluted protein (POSTN, sFRP-3 and MMP-7) should be as small as possible but in preferred case less than 40%.

### Example 9 Chronological Age Calculation

Instruction

The following nodes can be used for a description of the old and young plasma. Each node contains the combination of boolean statement which related to some parameter of the plasma. Parameters are encoded, the codes and units are described in the section ‘Codes of parameters’. The plasma can be described as ‘old’ if all statement of one node is true.

Codes of Parameters

ALT: SI (U/L) encoded as A{1}
Bilirubin, total (umol/L) encoded as A{2}
Direct HDL-Cholesterol (mmol/L) encoded as A{3}
GGT: SI (U/L) encoded as A{4}
Triglycerides (mmol/L) encoded as A{5}
Osmolality: SI (mmol/Kg) encoded as A{6}
Total Cholesterol(mmol/L) encoded as A{7}
Blood urea nitrogen (mmol/L) encoded as A{8}
Glycohemoglobin (%) encoded as A{9}
Blood lead (umol/L) encoded as A{10}
Phosphorus (mmol/L) encoded as A{11}
Creatinine (umol/L) encoded as A{12}
Chloride: SI (mmol/L) encoded as A{13}
Globulin (g/L) encoded as A{14}
Alkaline phosphatase(U/L) encoded as A{15}
Uric acid (umol/L) encoded as A{16}
Total calcium (mmol/L) encoded as A{17}
Glucose, serum (mmol/L) encoded as A{18}
Bicarbonate: SI (mmol/L) encoded as A{19}
Iron (umol/L) encoded as A{20}
Total protein (g/L) encoded as A{21}
Sodium: SI (mmol/L) encoded as A{22}
Lactate dehydrogenase LDH (U/L) encoded as A{23}
Potassium: SI (mmol/L) encoded as A{24}
Albumin (g/L) encoded as A{25}
Cotinine (ng/mL) encoded as A{26}
Blood cadmium (umol/L) encoded as A{27}
Iron (ug/dL) encoded as A{28}

Below are the examples of “aged” blood, e.g. blood Age=40 means that in below table the examples of blood represented of people aged 40 and elder, every row representing particular subject (individual).
blood Age=40

### (a) Paths

node number: 1126, A{9}>=5.45, A{10}>=0.0435, A{15}<175.5, A{9}<5.75, A{8}<5.18, A{7}>=4.59, A{27}>=1.825, A{3}<1.175, A{10}<0.0625, A{7}>=6.825 node number: 3522, A{9}<5.45, A{10}>=0.0585, A{7}<4.665, A{8}<5.705, A{25}>=42.5, A{15}<103.5, A{26}<0.0505, A{4}>=16.5, A{27}>=1.825, A{7}<4.385, A{21}<67.5

### (b) Examples

(i) For path #1126

| A{1} | A{2} | A{3} | A{4} | A{5} | A{6} | A{7} | A{8} | A{9} | A{10} | A{11} | A{12} | A{13} | A{14} | A{15} |
|---|---|---|---|---|---|---|---|---|---|---|---|---|---|---|
| 103 | 13.68 | 1.03 | 62 | 3.805 | 277 | 7.73 | 4.28 | 5.7 | 0.058 | 1.259 | 97.24 | 105 | 30 | 52 |
| 32 | 10.26 | 1.03 | 37 | 4.064 | 275 | 6.98 | 4.64 | 5.6 | 0.053 | 1.259 | 61.88 | 102 | 40 | 97 |
| 11 | 11.97 | 1.03 | 11 | 1.784 | 278 | 7.85 | 3.21 | 5.6 | 0.062 | 1.356 | 53.04 | 102 | 34 | 59 |
| 24 | 13.68 | 0.85 | 20 | 4.843 | 275 | 7.5 | 3.21 | 5.7 | 0.044 | 1.001 | 88.4 | 102 | 27 | 51 |
| 24 | 17.1 | 1.14 | 31 | 4.064 | 278 | 7.37 | 2.5 | 5.5 | 0.055 | 1.324 | 82.21 | 104 | 31 | 53 |
| 15 | 11.97 | 0.98 | 34 | 3.432 | 272 | 7.45 | 3.21 | 5.6 | 0.057 | 1.356 | 70.72 | 102 | 29 | 85 |
| 14 | 8.55 | 0.57 | 16 | 15.648 | 269 | 8.61 | 4.28 | 5.5 | 0.055 | 1.033 | 61.88 | 104 | 27 | 65 |
| 15 | 11.97 | 0.49 | 20 | 9.314 | 275 | 7.99 | 4.64 | 5.6 | 0.047 | 1.453 | 103.43 | 101 | 27 | 61 |
| 34 | 17.1 | 0.78 | 34 | 7.734 | 276 | 7.81 | 3.57 | 5.7 | 0.055 | 1.647 | 77.79 | 99 | 38 | 66 |
| 218 | 47.88 | 0.62 | 1116 | 30.404 | 279 | 13.65 | 5 | 5.6 | 0.051 | 1.001 | 95.47 | 103 | NA | 83 |
| 21 | 11.97 | 1.14 | 19 | 2.823 | 277 | 7.32 | 4.28 | 5.7 | 0.05 | 1.066 | 96.36 | 102 | 25 | 71 |
| 44 | 11.97 | 1.16 | 436 | 3.963 | 273 | 7.01 | 3.93 | 5.7 | 0.056 | 1.485 | 108.78 | 101 | 31 | 139 |

| A{16} | A{17} | A{18} | A{19} | A{20} | A{21} | A{22} | A{23} | A{24} | A{25} | A{26} | A{27} | A{28} |
|---|---|---|---|---|---|---|---|---|---|---|---|---|
| 434.2 | 2.375 | 5.44 | 22 | 28.5 | 71 | 139 | 169 | 4.1 | 41 | 342 | 2.67 | 159 |
| 380.7 | 2.5 | 5.16 | 27 | 6.3 | 74 | 138 | 141 | 4 | 34 | 0.04 | 5.34 | 35 |
| 356.9 | 2.35 | 4.88 | 24 | 13.8 | 71 | 140 | 93 | 4.2 | 37 | 284 | 7.83 | 77 |
| 297.4 | 2.25 | 5.77 | 26 | 10.7 | 68 | 138 | 122 | 4.1 | 41 | 204 | 4.27 | 80 |
| 356.9 | 2.525 | 4.5 | 25 | 18.3 | 76 | 141 | 124 | 4.2 | 45 | 0.032 | 3.02 | 102 |
| 350.9 | 2.475 | 5.38 | 25 | 21 | 71 | 137 | 92 | 4.3 | 42 | 295 | 5.34 | 117 |
| 279.6 | 2.2 | 5.11 | 22 | 5.6 | 67 | 135 | 144 | 4.2 | 40 | 93.5 | 3.47 | 31 |
| 327.1 | 2.35 | 5.11 | 26 | 8.1 | 73 | 138 | 344 | 4.5 | 46 | 336 | 4.89 | 45 |
| 291.5 | 2.45 | 4.61 | 30 | 9 | 78 | 139 | 133 | 4.5 | 40 | 0.024 | 4.45 | 50 |
| 416.4 | 2.5 | 6.05 | 22 | 47.5 | NA | 139 | 274 | 4.1 | 38 | 36.5 | 5.07 | 265 |
| 422.3 | 2.4 | 5.33 | 25 | 19 | 70 | 139 | 122 | 4.2 | 45 | 0.011 | 4.27 | 106 |
| 446.1 | 2.3 | 5.27 | 23 | 24.7 | 69 | 137 | 117 | 3.9 | 38 | 272 | 13.97 | 138 |

(ii) For path #3522

| A{1} | A{2} | A{3} | A{4} | A{5} | A{6} | A{7} | A{8} | A{9} | A{10} | A{11} | A{12} | A{13} | A{14} | A{15} |
|---|---|---|---|---|---|---|---|---|---|---|---|---|---|---|
| 36 | 23.94 | 1.58 | 44 | 0.745 | 273 | 4.16 | 3.93 | 5.2 | 0.063 | 1.033 | 88.4 | 101 | 22 | 76 |
| 22 | 17.1 | 1.29 | 19 | 1.264 | 279 | 3.93 | 3.21 | 5.1 | 0.217 | 1.292 | 70.72 | 106 | 23 | 78 |
| 24 | 20.52 | 1.76 | 23 | 0.497 | 271 | 3.7 | 5.36 | 5.1 | 0.121 | 1.13 | 88.4 | 99 | 24 | 46 |
| 30 | 13.68 | 1.6 | 27 | 1.253 | 277 | 4.29 | 4.28 | 5.1 | 0.204 | 1.292 | 79.56 | 107 | 21 | 101 |
| 19 | 20.52 | 2.15 | 21 | 0.384 | 252 | 4.37 | 2.5 | 4.8 | 0.109 | 1.356 | 88.4 | 94 | 20 | 51 |
| 28 | 15.39 | 1.16 | 29 | 0.914 | 273 | 4.01 | 4.28 | 5.4 | 0.087 | 0.936 | 90.17 | 103 | 21 | 53 |
| 23 | 32.49 | 1.45 | 20 | 0.914 | 278 | 3.49 | 5.36 | 5.2 | 0.14 | 1.259 | 89.28 | 104 | 24 | 72 |
| 21 | 8.55 | 2.02 | 26 | 0.632 | 279 | 4.09 | 3.21 | 5.4 | 0.062 | 1.582 | 74.26 | 106 | 21 | 69 |
| 44 | 15.39 | 1.29 | 103 | 1.569 | 277 | 4.01 | 5.36 | 5.1 | 0.088 | 1.13 | 95.47 | 106 | 24 | 82 |
| 20 | 11.97 | 1.27 | 18 | 1.31 | 281 | 4.24 | 4.64 | 5.1 | 0.082 | 1.421 | 68.07 | 108 | 24 | 61 |
| 25 | 15.39 | 1.86 | 20 | 0.768 | 285 | 3.62 | 3.93 | 5.1 | 0.098 | 1.066 | 90.17 | 107 | 21 | 58 |
| | | A{16} | A{17} | A{18} | A{19} | A{20} | A{21} | A{22} | A{23} | A{24} | A{25} | A{26} | A{27} | A{28} |
| | | 362.8 | 2.325 | 4.88 | 28 | 35.8 | 66 | 137 | 120 | 3.7 | 43 | 0.023 | 2.67 | 200 |
| | | 267.7 | 2.4 | 4.61 | 25 | 21.7 | 67 | 141 | 195 | 3.9 | 44 | 0.028 | 3.56 | 121 |
| | | 297.4 | 2.3 | 5.38 | 24 | 22.9 | 67 | 135 | 167 | 4.3 | 43 | 0.011 | 5.34 | 128 |
| | | 463.9 | 2.45 | 4.61 | 20 | 14.3 | 67 | 139 | 165 | 4.3 | 46 | 0.039 | 7.92 | 80 |
| | | 178.4 | 2.25 | 4.61 | 24 | 17.7 | 64 | 127 | 113 | 3.9 | 44 | 0.025 | 2.22 | 99 |
| | | 368.8 | 2.3 | 4.72 | 21 | 17.2 | 64 | 137 | 147 | 4 | 43 | 0.05 | 1.96 | 96 |
| | | 398.5 | 2.3 | 5.05 | 26 | 20.8 | 67 | 139 | 165 | 4.3 | 43 | 0.046 | 4.8 | 116 |
| | | 249.8 | 2.35 | 4.5 | 26 | 5.9 | 66 | 141 | 127 | 4.4 | 45 | 0.016 | 4.27 | 33 |
| | | 410.4 | 2.25 | 5.88 | 21 | 19.9 | 67 | 138 | 167 | 4.2 | 43 | 0.019 | 2.22 | 111 |
| | | 249.8 | 2.3 | 5.05 | 26 | 6.4 | 67 | 141 | 137 | 4 | 43 | 0.021 | 2.85 | 36 |
| | | 327.1 | 2.4 | 5.72 | 27 | 20.2 | 67 | 143 | 112 | 4 | 46 | 0.031 | 3.56 | 113 |

Age=50

### (c) Paths

node number: 1172, A{9}>=5.55, A{8}>=4.82, A{8}<6.085, A{9}>=5.75, A{1}<32.5, A{10}<0.0485, A{18}>=5.525, A{26}>=0.256, A{27}>=2.98, A{1}<22.5
node number: 1220, A{9}>=5.55, A{8}>=4.82, A{8}<6.085, A{9}<5.75, A{27}>=2.18, A{26}<0.9155, A{1}<34.5, A{5}<0.7055, A{7}>=4.785, A{19}>=26.5
node number: 21160, A{9}>=5.55, A{8}<4.82, A{10}>=0.0495, A{9}>=5.85, A{1}>=31.5, A{27}>=2.005, A{26}>=0.0225, A{20}>=11.72, A{14}<35.5, A{16}<413.4, A{3}<1.33, A{1}<103.5, A{10}>=0.09, A{26}<0.042
node number: 11120, A{9}>=5.55, A{8}<4.82, A{10}>=0.0495, A{9}<5.85, A{7}<4.955, A{11}<1.435, A{10}<0.0845, A{19}<26.5, A{26}>=0.039, A{6}>=277.5, A{21}<70.5, A{14}>=25.5, A{13}<106.5
node number: 392, A{9}<5.55, A{10}>=0.0685, A{8}>=5.705, A{25}<44.5, A{27}<2.095, A{26}<0.0695, A{24}>=3.935, A{7}>=5.805
node number: 13508, A{9}<5.55, A{10}>=0.0685, A{8}<5.705, A{7}>=4.98, A{1}<33.5, A{26}>=0.0825, A{25}>=43.5, A{18}>=5.109, A{7}>=5.16, A{27}>=3.2, A{3}<1.885, A{6}>=279.5, A{1}<16.5
node number: 6968, A{9}<5.55, A{10}>=0.0685, A{8}<5.705, A{7}<4.98, A{25}<42.5, A{12}>=61.89, A{26}>=0.0625, A{10}<0.1075, A{6}<281.5, A{7}>=4.355, A{9}>=5.35, A{26}<0.2905
node number: 872, A{9}<5.55, A{10}>=0.0685, A{8}<5.705, A{7}<4.98, A{25}<42.5, A{12}<61.89, A{19}>=26.5, A{11}<1.435, A{26}<0.174

### (d) Examples

(i) For path #1172

| A{1} | A{2} | A{3} | A{4} | A{5} | A{6} | A{7} | A{8} | A{9} | A{10} | A{11} | A{12} | A{13} | A{14} | A{15} |
|---|---|---|---|---|---|---|---|---|---|---|---|---|---|---|
| 15 | 10.26 | NA | 25 | 2.653 | 271 | 4.53 | 5 | 9.1 | 0.014 | 1.52 | 44.2 | 98 | 35 | NA |
| 21 | 8.55 | NA | 148 | 6.322 | 281 | 5.17 | 5 | 6.8 | 0.034 | 0.9 | 44.2 | 101 | 37 | NA |
| 17 | 15.39 | 1.24 | 17 | 0.869 | 270 | 5.33 | 5 | 7.3 | 0.048 | 1.066 | 79.56 | 102 | 26 | 72 |
| 20 | 18.81 | 0.8 | 43 | 1.456 | 274 | 4.97 | 6.07 | 6.6 | 0.019 | 1.162 | 63.65 | 99 | 34 | 94 |
| 20 | 10.26 | 1.55 | 17 | 1.423 | 275 | 5.46 | 5.71 | 6.6 | 0.045 | 1.388 | 74.26 | 97 | 29 | 61 |
| 20 | 10.26 | 0.93 | 24 | 2.597 | 277 | 4.42 | 6.07 | 6.1 | 0.032 | 1.227 | 85.75 | 98 | 27 | 45 |
| 16 | 15.39 | 1.99 | 19 | 0.971 | 286 | 3.98 | 5.71 | 10.2 | 0.024 | 1.227 | 68.07 | 104 | 27 | 54 |
| 22 | 10.26 | 1.03 | 19 | 1.795 | 285 | 4.76 | 5 | 6.1 | 0.048 | 1.227 | 81.33 | 104 | 26 | 84 |
| 12 | 8.55 | 1.78 | 16 | 1.332 | 283 | 4.06 | 6.07 | 7.1 | 0.044 | 1.227 | 85.75 | 103 | 28 | 69 |
| 11 | 11.97 | 1.6 | 16 | 1.118 | 276 | 6.28 | 5.71 | 6 | 0.043 | 1.421 | 65.42 | 103 | 32 | 76 |
| 20 | 10.26 | 0.98 | 32 | 2.258 | 284 | 4.37 | 5.36 | 6.1 | 0.047 | 1.098 | 51.27 | 108 | 29 | 101 |
| 12 | 6.84 | 1.19 | 27 | 2.36 | 285 | 4.29 | 5.36 | 6.5 | 0.041 | 0.904 | 86.63 | 108 | 29 | 89 |
| 16 | 10.26 | 1.27 | 21 | 3.218 | 279 | 5.33 | 5.71 | 11.1 | 0.045 | 1.098 | 76.91 | 96 | 33 | 90 |
| 21 | 10.26 | 2.15 | 27 | 1.106 | 282 | 5.38 | 5.36 | 5.8 | 0.036 | 1.13 | 69.84 | 105 | 27 | 64 |
| 12 | 6.84 | 1.34 | 9 | 2.529 | 284 | 5.09 | 6.07 | 6.1 | 0.023 | 1.55 | 76.91 | 105 | 32 | 32 |
| | | A{16} | A{17} | A{18} | A{19} | A{20} | A{21} | A{22} | A{23} | A{24} | A{25} | A{26} | A{27} | A{28} |
| | | 202.2 | 2.35 | 7.882 | 26 | 11.46 | 71 | 134 | NA | 4 | 36 | 86.6 | 3.56 | 64 |
| | | 309.3 | 2.325 | 8.604 | 23 | 20.95 | 79 | 139 | NA | 4.1 | 42 | 89.2 | 8.01 | 117 |
| | | 297.4 | 2.375 | 9.1 | 26 | 18.1 | 73 | 137 | 148 | 4.3 | 47 | 0.868 | 3.02 | 101 |
| | | 428.3 | 2.35 | 6.11 | 26 | 25.1 | 78 | 136 | 105 | 3.7 | 44 | 0.367 | 4.27 | 140 |
| | | 315.2 | 2.375 | 11.32 | 23 | 7.3 | 71 | 134 | 156 | 3.5 | 42 | 0.726 | 3.11 | 41 |
| | | 410.4 | 2.275 | 5.72 | 28 | 9.1 | 72 | 138 | 129 | 3.1 | 45 | 0.792 | 9.43 | 51 |
| | | 303.3 | 2.425 | 10.82 | 29 | 16.3 | 74 | 140 | 112 | 4.3 | 47 | 0.422 | 4.54 | 91 |
| | | 392.6 | 2.35 | 6.38 | 27 | 13.6 | 65 | 142 | 163 | 3.8 | 39 | 240 | 4.45 | 76 |
| | | 291.5 | 2.375 | 7.77 | 27 | 20.1 | 68 | 140 | 136 | 3.6 | 40 | 281 | 10.68 | 112 |
| | | 279.6 | 2.2 | 6.83 | 25 | 14.1 | 71 | 138 | 129 | 3.6 | 39 | 343 | 7.47 | 79 |
| | | 309.3 | 2.25 | 5.66 | 25 | 14.3 | 69 | 142 | 128 | 3.8 | 40 | 371 | 4.98 | 80 |
| | | 452 | 2.375 | 6.88 | 25 | 5.4 | 68 | 142 | 104 | 3.7 | 39 | 192 | 12.72 | 30 |
| | | 237.9 | 2.3 | 12.88 | 29 | 10 | 72 | 135 | 190 | 4.3 | 39 | 1.18 | 4.54 | 56 |
| | | 422.3 | 2.375 | 5.66 | 25 | 21.3 | 70 | 141 | 130 | 4.3 | 43 | 5.38 | 4.54 | 119 |
| | | 309.3 | 2.525 | 6.27 | 27 | 15.6 | 73 | 141 | 115 | 3.8 | 41 | 126 | 5.34 | 87 |

(ii) For path #1220

| A{1} | A{2} | A{3} | A{4} | A{5} | A{6} | A{7} | A{8} | A{9} | A{10} | A{11} | A{12} | A{13} | A{14} | A{15} |
|---|---|---|---|---|---|---|---|---|---|---|---|---|---|---|
| 15 | 8.55 | 2.02 | 15 | 0.553 | 276 | 5.77 | 6.07 | 5.7 | 0.082 | 1.098 | 79.56 | 99 | 29 | 50 |
| 15 | 10.26 | 2.3 | 14 | 0.531 | 282 | 5.09 | 5.71 | 5.7 | 0.039 | 1.195 | 88.4 | 102 | 27 | 46 |
| 21 | 11.97 | 1.94 | 14 | 0.689 | 273 | 5.53 | 5.36 | 5.7 | 0.101 | 1.162 | 79.56 | 96 | 30 | 57 |
| 17 | 13.68 | 1.86 | 30 | 0.866 | 282 | 5.09 | 5.71 | 5.6 | 0.27 | 1.259 | 97.24 | 102 | 31 | 82 |
| 19 | 11.97 | 2.56 | 12 | 0.61 | 289 | 5.35 | 5 | 5.7 | 0.085 | 1.324 | 114.04 | 107 | 29 | 49 |
| 12 | 15.39 | 1.4 | 15 | 0.542 | 273 | 4.86 | 5 | 5.7 | 0.252 | 0.71 | 76.02 | 102 | 33 | 70 |
| 27 | 15.39 | 2.61 | 19 | 0.373 | 278 | 5.17 | 5 | 5.7 | 0.096 | 1.098 | 68.95 | 104 | 28 | 55 |
| 20 | 32.49 | 1.78 | 17 | 0.632 | 286 | 4.84 | 5.36 | 5.7 | 0.088 | 1.033 | 99.01 | 105 | 25 | 47 |
| 15 | 10.26 | 2.4 | 13 | 0.61 | 285 | 4.89 | 5.36 | 5.7 | 0.077 | 1.259 | 63.65 | 105 | 25 | 115 |
| 22 | 11.97 | 2.9 | 33 | 0.655 | 284 | 5.46 | 6.07 | 5.7 | 0.093 | 1.227 | 77.79 | 103 | 29 | 60 |
| 19 | 15.39 | 2.46 | 15 | 0.576 | 281 | 4.91 | 5 | 5.7 | 0.099 | 1.227 | 58.34 | 103 | 27 | 38 |
| 21 | 17.1 | 1.97 | 15 | 0.508 | 278 | 6.18 | 5.71 | 5.6 | 0.204 | 1.098 | 76.02 | 104 | 32 | 52 |
| 24 | 11.97 | 1.97 | 37 | 0.485 | 273 | 5.82 | 6.07 | 5.7 | 0.063 | 1.324 | 74.26 | 96 | 36 | 102 |
| 13 | 22.23 | 3.57 | 10 | 0.565 | 272 | 7.55 | 5.36 | 5.7 | 0.058 | 1.485 | 75.14 | 97 | 27 | 54 |
| 11 | 11.97 | 2.46 | 17 | 0.542 | 278 | 6.78 | 5.71 | 5.6 | 0.113 | 1.162 | 91.05 | 99 | 32 | 77 |
| 21 | 10.26 | 2.02 | 31 | 0.632 | 283 | 5.48 | 5.71 | 5.7 | 0.06 | 1.356 | 106.96 | 102 | 24 | 52 |
| 19 | 13.68 | 1.86 | 23 | 0.44 | 277 | 5.35 | 5.36 | 5.7 | 0.049 | 0.969 | 121.11 | 103 | 24 | 54 |
| 16 | 15.39 | 1.5 | 22 | 0.666 | 286 | 5.15 | 5 | 5.6 | 0.063 | 1.098 | 103.43 | 105 | 29 | 59 |
| | | A{16} | A{17} | A{18} | A{19} | A{20} | A{21} | A{22} | A{23} | A{24} | A{25} | A{26} | A{27} | A{28} |
| | | 398.5 | 2.6 | 6.27 | 27 | 19.3 | 69 | 137 | 126 | 3.9 | 39 | 0.034 | 5.34 | 108 |
| | | 345 | 2.425 | 5.05 | 28 | 16.5 | 70 | 141 | 163 | 4.2 | 43 | 0.011 | 6.23 | 92 |
| | | 255.8 | 2.25 | 5.5 | 30 | 9.9 | 68 | 136 | 179 | 4 | 38 | 0.202 | 4.45 | 55 |
| | | 333.1 | 2.425 | 4.77 | 29 | 12.9 | 75 | 141 | 143 | 3.6 | 44 | 0.049 | 2.85 | 72 |
| | | 339 | 2.425 | 5.16 | 28 | 17.7 | 71 | 145 | 134 | 4.6 | 42 | 0.011 | 2.85 | 99 |
| | | 243.9 | 2.55 | 4.61 | 27 | 8.8 | 73 | 137 | 104 | 3.6 | 40 | 0.276 | 3.58 | 49 |
| | | 356.9 | 2.35 | 5.77 | 28 | 24.7 | 71 | 139 | 267 | 5 | 43 | 0.564 | 4.27 | 138 |
| | | 356.9 | 2.4 | 5.55 | 30 | 33.5 | 69 | 143 | 130 | 4.2 | 44 | 0.011 | 2.4 | 187 |
| | | 303.3 | 2.275 | 4.83 | 32 | 8.4 | 63 | 143 | 153 | 3.7 | 38 | 0.022 | 4.98 | 47 |
| | | 285.5 | 2.325 | 4.55 | 27 | 16.5 | 73 | 142 | 165 | 4.2 | 44 | 0.011 | 2.94 | 92 |
| | | 291.5 | 2.35 | 4.72 | 27 | 18.3 | 71 | 141 | 145 | 4.1 | 44 | 0.011 | 4.89 | 102 |
| | | 350.9 | 2.325 | 4.72 | 27 | 13.3 | 77 | 139 | 131 | 3.5 | 45 | 0.021 | 3.2 | 74 |
| | | 398.5 | 2.525 | 4.72 | 27 | 14.7 | 76 | 136 | 159 | 3.6 | 40 | 0.025 | 3.38 | 82 |
| | | 202.2 | 2.625 | 4.44 | 27 | 17.9 | 77 | 136 | 107 | 4 | 50 | 0.037 | 2.94 | 100 |
| | | 333.1 | 2.4 | 4.66 | 28 | 10.7 | 74 | 139 | 176 | 3.4 | 42 | 0.159 | 4.36 | 60 |
| | | 410.4 | 2.375 | 4.5 | 30 | 27.2 | 68 | 142 | 146 | 4.5 | 44 | 0.02 | 2.49 | 152 |
| | | 315.2 | 2.3 | 4.11 | 27 | 14.9 | 67 | 139 | 139 | 4.1 | 43 | 0.043 | 3.02 | 83 |
| | | 398.5 | 2.375 | 5.61 | 27 | 16.5 | 72 | 143 | 124 | 3.9 | 43 | 0.091 | 2.22 | 92 |

Age=60

### (e) Paths

node number: 2178, A{9}>=5.65, A{8}>=5.18, A{1}<26.5, A{8}<6.95, A{27}>=2.18, A{26}<7.725, A{23}>=130.5, A{27}>=3.065, A{11}<1.275, A{2}<5.965, A{7}<4.215
node number: 4802, A{9}>=5.65, A{8}>=5.18, A{1}>=26.5, A{6}>=280.5, A{7}>=4.77, A{8}<9.105, A{26}<0.1525, A{27}>=2.18, A{7}<6.48, A{24}>=3.69, A{9}<6.15, A{3}>=1.795
node number: 19652, A{9}>=5.65, A{8}>=5.18, A{1}>=26.5, A{6}<280.5, A{27}>=1.825, A{26}<2.31, A{7}>=4.64, A{27}<6.005, A{20}>=15.68, A{9}>=5.75, A{1}<51.5, A{23}<135.5, A{18}>=5.91, A{2}>=12.82
node number: 10278, A{9}>=5.65, A{8}<5.18, A{10}>=0.0435, A{1}<27.5, A{26}<0.1625, A{12}>=69.4, A{27}>=2.625, A{24}<4.195, A{5}>=1.191, A{5}<2.625, A{19}<27.5, A{10}<0.1105, A{21}<66.5
node number: 10440, A{9}>=5.65, A{8}<5.18, A{10}>=0.0435, A{1}<27.5, A{26}<0.1625, A{12}<69.4, A{19}<24.5, A{2}>=5.965, A{5}>=1.022, A{22}<140.1, A{19}>=22.5, A{11}<1.146, A{26}<0.016
node number: 21250, A{9}>=5.65, A{8}<5.18, A{10}>=0.0435, A{1}<27.5, A{26}>=0.1625, A{10}<0.1075, A{9}>=5.95, A{8}>=3.39, A{3}>=1.02, A{11}<1.308, A{20}<18.81, A{2}>=9.43, A{8}<4.62, A{4}>=27.5
node number: 832, A{9}<5.65, A{8}>=5.89, A{27}<2.18, A{10}>=0.0745, A{25}<44.5, A{1}<26.5, A{5}>=0.858, A{10}>=0.0945, A{26}<0.013

### (f) Examples

(i) For path #?178

| A{1} | A{2} | A{3} | A{4} | A{5} | A{6} | A{7} | A{8} | A{9} | A{10} | A{11} | A{12} | A{13} | A{14} | A{15} |
|---|---|---|---|---|---|---|---|---|---|---|---|---|---|---|
| 20 | 5.1 | NA | 71 | 0.813 | 279 | 3.26 | 6.1 | 6 | 0.164 | 0.969 | 79.6 | 97.8 | 39 | 115 |
| 22 | 5.1 | NA | 24 | 0.982 | 279 | 4.01 | 6.1 | 5.8 | 0.097 | 0.969 | 44.2 | 101.1 | 32 | 89 |
| 15 | 5.1 | NA | 16 | 1.377 | 276 | 4.09 | 5.4 | 5.7 | 0.068 | 1.033 | 70.7 | 100.9 | 43 | 63 |
| 18 | 1.71 | 1.14 | 15 | 1.818 | 277 | 3.9 | 6.43 | 6.1 | 0.101 | 1.259 | 97.24 | 106 | 31 | 74 |
| 14 | 3.42 | 1.53 | 12 | 2.055 | 280 | 3.78 | 5.71 | 5.7 | 0.057 | 1.259 | 84.86 | 105 | 21 | 69 |
| 37 | 13.68 | 1.81 | 100 | 1.637 | 284 | 6.39 | 6.43 | 6 | 0.058 | 1.421 | 61.88 | 104 | 21 | 91 |
| 31 | 20.52 | 1.81 | 16 | 0.948 | 285 | 5.09 | 6.07 | 5.9 | 0.087 | 1.033 | 99.01 | 103 | 27 | 44 |
| 28 | 10.26 | 1.97 | 11 | 0.982 | 285 | 4.84 | 5.36 | 5.7 | 0.165 | 1.421 | 58.34 | 103 | 24 | 65 |
| 97 | 10.26 | 1.97 | 132 | 1.231 | 282 | 6.05 | 7.85 | 6.1 | 0.053 | 1.453 | 53.92 | 105 | 26 | 86 |
| 31 | 20.52 | 2.09 | 21 | 0.948 | 282 | 4.81 | 5.71 | 6 | 0.072 | 1.162 | 77.79 | 101 | 30 | 103 |
| 37 | 23.94 | 1.91 | 78 | 1.535 | 282 | 5.48 | 6.07 | 5.9 | 0.068 | 1.098 | 61 | 107 | 23 | 54 |
| 29 | 8.55 | 1.81 | 18 | 1.027 | 282 | 5.12 | 7.85 | 6.1 | 0.092 | 1.324 | 77.79 | 100 | 33 | 88 |
| 30 | 10.26 | 1.94 | 18 | 1.502 | 288 | 6.34 | 7.5 | 5.9 | 0.031 | 1.066 | 67.18 | 104 | 25 | 43 |
| 27 | 8.55 | 1.91 | 17 | 1.411 | 285 | 6.36 | 6.43 | 5.8 | 0.095 | 1.518 | 92.82 | 106 | 25 | 90 |
| 40 | 11.97 | 2.33 | 61 | 0.621 | 283 | 4.81 | 6.07 | 5.7 | 0.249 | 1.098 | 76.02 | 100 | 28 | 43 |
| | | A{16} | A{17} | A{18} | A{19} | A{20} | A{21} | A{22} | A{23} | A{24} | A{25} | A{26} | A{27} | A{28} |
| | | 392.6 | 2.475 | 7.494 | 27 | 16.48 | 80 | 137.6 | 163 | 4.25 | 41 | 0.14 | 4.45 | 92 |
| | | 434.2 | 2.375 | 4.996 | 23 | 18.27 | 75 | 139.2 | 172 | 3.81 | 43 | 0.11 | 5.34 | 102 |
| | | 220.1 | 2.4 | 5.051 | 25 | 11.82 | 83 | 138.2 | 136 | 3.56 | 40 | 0.035 | 8.01 | 66 |
| | | 422.3 | 2.325 | 5.05 | 24 | 12.7 | 68 | 138 | 167 | 5 | 37 | 0.169 | 4.45 | 71 |
| | | 243.9 | 2.35 | 6.44 | 24 | 11.8 | 60 | 139 | 132 | 4.3 | 39 | 0.011 | 5.87 | 66 |
| | | 285.5 | 2.35 | 4.72 | 27 | 14 | 70 | 142 | 163 | 3.8 | 49 | 0.011 | 2.67 | 78 |
| | | 505.6 | 2.3 | 5.72 | 28 | 19 | 74 | 142 | 164 | 3.8 | 47 | 0.073 | 2.94 | 106 |
| | | 249.8 | 2.45 | 8.16 | 28 | 10.7 | 66 | 141 | 130 | 4 | 42 | 0.026 | 5.69 | 60 |
| | | 273.6 | 2.45 | 4.77 | 27 | 14.5 | 69 | 140 | 169 | 4 | 43 | 0.143 | 2.49 | 81 |
| | | 368.8 | 2.45 | 4.88 | 30 | 14 | 77 | 141 | 151 | 3.8 | 47 | 0.011 | 5.96 | 78 |
| | | 285.5 | 2.325 | 6.44 | 26 | 37.8 | 63 | 140 | 134 | 3.9 | 40 | 0.015 | 3.11 | 211 |
| | | 303.3 | 2.325 | 5.05 | 29 | 11.3 | 72 | 140 | 243 | 4 | 39 | 0.011 | 2.4 | 63 |
| | | 243.9 | 2.425 | 5.5 | 27 | 10.6 | 70 | 143 | 150 | 3.9 | 45 | 0.011 | 4.54 | 59 |
| | | 273.6 | 2.4 | 5.61 | 27 | 12.7 | 62 | 142 | 137 | 4.9 | 37 | 0.011 | 4 | 71 |
| | | 440.2 | 2.425 | 7.11 | 28 | 20.6 | 72 | 140 | 187 | 4.2 | 44 | 0.011 | 3.2 | 115 |

(ii) For path #4802 (above)

Age=60

### (g) Paths

node number: 16434, A{8}>=6.605, A{12}>=96.78, A{1}<24.5, A{27}>=2.625, A{26}<1.445, A{12}<310.7, A{24}>=4.095, A{9}<7.25, A{10}<0.1205, A{7}>=4.98, A{21}<74.5, A{5}>=0.8635, A{24}<4.39, A{26}<0.0165
node number: 3080, A{8}<6.605, A{9}>=5.55, A{8}>=4.82, A{1}<22.5, A{27}>=2.265, A{26}<3.88, A{12}>=76.47, A{27}<4.225, A{21}<72.5, A{1}<19.5, A{4}>=40
node number: 24720, A{8}<6.605, A{9}>=5.55, A{8}>=4.82, A{1}<22.5, A{27}>=2.265, A{26}<3.88, A{12}<76.47, A{24}>=4.195, A{12}>=60.55, A{10}<0.09, A{5}>=1.304, A{26}<0.0265, A{25}<43.5, A{4}<17.5
node number: 3208, A{8}<6.605, A{9}>=5.55, A{8}>=4.82, A{1}>=22.5, A{26}<0.0685, A{27}>=3.425, A{12}>=70.71, A{7}>=4.85, A{24}>=4.13, A{27}>=3.915, A{4}<18.5
node number: 52032, A{8}<6.605, A{9}>=5.55, A{8}>=4.82, A{1}>=22.5, A{26}<0.0685, A{27}<3.425, A{7}>=3.605, A{1}<37.5, A{10}<0.0795, A{18}>=5.161, A{3}>=1.28, A{26}<0.013, A{24}>=3.93, A{23}>=123.5, A{18}<5.745
node number: 7264, A{8}<6.605, A{9}<5.55, A{10}>=0.0795, A{26}<0.0695, A{27}>=3.785, A{12}<79.12, A{12}<61.89, A{13}<103.5, A{7}>=4.885, A{1}<18.5, A{2}>=6.82, A{24}>=4.11
node number: 3648, A{8}<6.605, A{9}<5.55, A{10}>=0.0795, A{26}<0.0695, A{27}<3.785, A{12}>=68.51, A{1}<22.5, A{18}>=5.025, A{24}>=4.215, A{10}>=0.091, A{11}>=1.

### (h) Examples

(i) For path #16434

| A{1} | A{2} | A{3} | A{4} | A{5} | A{6} | A{7} | A{8} | A{9} | A{10} | A{11} | A{12} | A{13} | A{14} | A{15} |
|---|---|---|---|---|---|---|---|---|---|---|---|---|---|---|
| 21 | 10.28 | NA | 37 | 2.834 | 284 | 5.35 | 7.85 | 5.3 | 0.068 | 1.1 | 106.08 | 103 | 28 | NA |
| 14 | 10.26 | 1.01 | 11 | 3.116 | 284 | 5.77 | 7.5 | 5.6 | 0.053 | 1.227 | 114.92 | 107 | 25 | 92 |
| 21 | 15.39 | 1.27 | 10 | 1.671 | 278 | 5.84 | 8.93 | 5.2 | 0.101 | 1.162 | 212.16 | 102 | 28 | 62 |
| 23 | 17.1 | 1.24 | 44 | 2.732 | 280 | 6.34 | 5.78 | 5.3 | 0.068 | 1.227 | 132.6 | 105 | 25 | 78 |
| 18 | 20.52 | 1.58 | 9 | 1.897 | 280 | 5.4 | 7.14 | 5.5 | 0.07 | 1.195 | 123.76 | 102 | 22 | 51 |
| 20 | 8.55 | 1.29 | 37 | 2.348 | 282 | 5.28 | 8.57 | 5.7 | 0.099 | 0.936 | 97.24 | 103 | 24 | 62 |
| 13 | 15.39 | 1.81 | 19 | 1.848 | 282 | 4.99 | 8.57 | 5.4 | 0.054 | 1.162 | 106.08 | 105 | 25 | 84 |
| 16 | 10.26 | 1.32 | 28 | 2.631 | 282 | 7.03 | 8.57 | 6 | 0.046 | 1.033 | 99.89 | 104 | 21 | 44 |
| 18 | 11.97 | 1.34 | 26 | 1.829 | 287 | 5.84 | 7.14 | 6.1 | 0.077 | 1.292 | 101.66 | 106 | 22 | 35 |
| 16 | 20.52 | 1.42 | 12 | 1.298 | 286 | 5.28 | 11.07 | 6 | 0.103 | 1.033 | 176.8 | 166 | 30 | 58 |
| 20 | 15.39 | 1.47 | 31 | 2.055 | 277 | 6.08 | 7.85 | 6.3 | 0.06 | 1.679 | 100.78 | 101 | 26 | 41 |
| 20 | 13.68 | 1.94 | 27 | 1.592 | 268 | 9.98 | 7.5 | 5.5 | 0.058 | 1.13 | 98.12 | 96 | 23 | 61 |
| 18 | 8.55 | 1.34 | 18 | 1.863 | 292 | 5.56 | 11.07 | 6 | 0.119 | 0.936 | 99.89 | 103 | 29 | 112 |
| 19 | 15.39 | 1.09 | 15 | 2.461 | 289 | 5.95 | 10.35 | 5.5 | 0.117 | 1.518 | 154.7 | 102 | 22 | 61 |
| 9 | 6.84 | 1.47 | 8 | 1.344 | 281 | 5.82 | 7.85 | 5.6 | 0.09 | 1.227 | 109.62 | 104 | 23 | 98 |
| 19 | 11.97 | 1.29 | 48 | 2.676 | 290 | 5.4 | 7.5 | 5.6 | 0.05 | 0.936 | 113.15 | 109 | 19 | 83 |
| 20 | 17.1 | 1.09 | 19 | 1.569 | 285 | 5.12 | 9.28 | 5.9 | 0.042 | 1.098 | 123.76 | 105 | 30 | 51 |
| 17 | 10.26 | 1.37 | 15 | 1.908 | 277 | 5.09 | 8.21 | 5.6 | 0.085 | 0.969 | 112.27 | 103 | 25 | 48 |
| | | A{16} | A{17} | A{18} | A{19} | A{20} | A{21} | A{22} | A{23} | A{24} | A{25} | A{26} | A{27} | A{28} |
| | | 547.2 | 2.35 | 4.663 | 23 | 17.01 | 70 | 141 | NA | 4.1 | 42 | 0.011 | 3.58 | 95 |
| | | 428.3 | 2.425 | 5.27 | 25 | 8.1 | 66 | 141 | 100 | 4.2 | 41 | 0.011 | 6.23 | 45 |
| | | 422.3 | 2.45 | 5.11 | 21 | 11.3 | 72 | 137 | 135 | 4.2 | 44 | 0.016 | 9.79 | 83 |
| | | 505.6 | 2.375 | 4.44 | 26 | 12.4 | 65 | 140 | 146 | 4.2 | 40 | 0.011 | 2.67 | 89 |
| | | 279.6 | 2.35 | 4.94 | 27 | 13.3 | 67 | 139 | 171 | 4.1 | 44 | 0.011 | 3.65 | 74 |
| | | 368.8 | 2.225 | 7.6 | 24 | 26.9 | 63 | 138 | 145 | 4.3 | 39 | 0.011 | 5.34 | 150 |
| | | 386.6 | 2.275 | 4.22 | 24 | 7.2 | 65 | 140 | 158 | 4.3 | 40 | 0.011 | 5.43 | 40 |
| | | 398.5 | 2.3 | 5.44 | 28 | 11.3 | 65 | 139 | 131 | 4.2 | 44 | 0.011 | 7.12 | 63 |
| | | 553.2 | 2.325 | 4.94 | 29 | 18.3 | 62 | 143 | 150 | 4.1 | 40 | 0.011 | 4.63 | 102 |
| | | 410.4 | 2.325 | 5.88 | 24 | 19.7 | 70 | 140 | 149 | 4.3 | 40 | 0.011 | 3.38 | 110 |
| | | 416.4 | 2.425 | 5 | 25 | 18.6 | 70 | 137 | 147 | 4.3 | 44 | 0.011 | 5.16 | 104 |
| | | 232 | 2.675 | 4.44 | 28 | 14.5 | 69 | 133 | 152 | 4.2 | 46 | 0.011 | 4.98 | 81 |
| | | 422.3 | 2.3 | 7.66 | 27 | 10.2 | 69 | 142 | 132 | 4.2 | 40 | 0.011 | 4.98 | 57 |
| | | 410.4 | 2.275 | 9.16 | 28 | 17.7 | 64 | 140 | 165 | 4.3 | 42 | 0.011 | 7.38 | 99 |
| | | 333.1 | 2.275 | 3.66 | 25 | 11.6 | 66 | 140 | 149 | 4.1 | 43 | 0.011 | 4.8 | 65 |
| | | 410.4 | 2.225 | 7.55 | 22 | 13.4 | 58 | 143 | 119 | 4.2 | 39 | 0.011 | 3.65 | 75 |
| | | 511.5 | 2.375 | 6.72 | 23 | 17.6 | 72 | 140 | 130 | 4.2 | 42 | 0.011 | 5.16 | 98 |
| | | 261.7 | 2.617 | 5.11 | 23 | 15 | 67 | 137 | 125 | 4.3 | 42 | 0.011 | 3.2 | 84 |

(ii) For path #3080

| A{1} | A{2} | A{3} | A{4} | A{5} | A{6} | A{7} | A{8} | A{9} | A{10} | A{11} | A{12} | A{13} | A{14} | A{15} |
|---|---|---|---|---|---|---|---|---|---|---|---|---|---|---|
| 19 | 8.6 | NA | 121 | 1.073 | 283 | 5.74 | 5 | 6 | 0.145 | 1.033 | 88.4 | 106.5 | 29 | 87 |
| 18 | 17.1 | NA | 45 | 0.847 | 287 | 5.69 | 6.43 | 5.6 | 0.082 | 1 | 97.24 | 103 | 30 | NA |
| 14 | 8.55 | 1.55 | 45 | 1.445 | 275 | 6 | 6.07 | 5.8 | 0.053 | 1.13 | 88.4 | 104 | 28 | 63 |
| 15 | 10.26 | 1.76 | 43 | 1.219 | 282 | 5.2 | 6.43 | 5.6 | 0.082 | 1.324 | 79.56 | 103 | 28 | 95 |
| 16 | 8.56 | 1.55 | 57 | 1.739 | 284 | 6.41 | 6.43 | 5.7 | 0.117 | 0.936 | 123.76 | 104 | 31 | 79 |
| 19 | 13.68 | 1.86 | 45 | 2.348 | 278 | 5.33 | 5.36 | 9 | 0.167 | 1.033 | 97.24 | 102 | 27 | 85 |
| 13 | 8.55 | 1.37 | 57 | 2.845 | 281 | 6.8 | 5.71 | 5.6 | 0.236 | 1.485 | 81.33 | 103 | 32 | 88 |
| 16 | 11.97 | 1.09 | 71 | 0.485 | 275 | 2.95 | 6.07 | 6.9 | 0.053 | 1.485 | 90.17 | 101 | 30 | 90 |
| 18 | 10.26 | 2.07 | 47 | 1.682 | 280 | 5.64 | 6.43 | 5.7 | 0.048 | 1.356 | 90.17 | 104 | 26 | 54 |
| 14 | 23.94 | 1.27 | 65 | 1.558 | 280 | 4.47 | 5.36 | 8.2 | 0.032 | 1.195 | 103.43 | 103 | 30 | 153 |
| | | A{16} | A{17} | A{18} | A{19} | A{20} | A{21} | A{22} | A{23} | A{24} | A{25} | A{26} | A{27} | A{28} |
| | | 279.6 | 2.4 | 7.827 | 24 | 13.79 | 71 | 140.3 | 108 | 4.39 | 42 | 0.035 | 2.67 | 77 |
| | | 327.1 | 2.3 | 5.162 | 26 | 17.91 | 71 | 143 | NA | 4.5 | 41 | 0.011 | 3.56 | 100 |
| | | 255.8 | 2.425 | 5.33 | 25 | 13.8 | 70 | 137 | 131 | 4.3 | 42 | 0.137 | 3.56 | 77 |
| | | 392.6 | 2.3 | 5.77 | 26 | 13.8 | 71 | 140 | 234 | 4.2 | 43 | 0.05 | 2.67 | 77 |
| | | 196.3 | 2.375 | 6 | 28 | 7.3 | 67 | 141 | 136 | 3.6 | 36 | 0.034 | 3.91 | 41 |
| | | 190.3 | 2.475 | 8.49 | 24 | 19.3 | 70 | 137 | 148 | 4.1 | 43 | 0.038 | 4 | 108 |
| | | 374.7 | 2.4 | 5.55 | 26 | 15.4 | 68 | 140 | 114 | 4 | 36 | 0.011 | 3.47 | 56 |
| | | 226 | 2.3 | 4.72 | 24 | 11.5 | 70 | 137 | 136 | 3.9 | 40 | 0.299 | 2.67 | 64 |
| | | 303.3 | 2.425 | 5.66 | 25 | 13.8 | 65 | 139 | 109 | 4.2 | 39 | 0.345 | 4 | 77 |
| | | 463.9 | 2.2 | 8.55 | 26 | 11.1 | 66 | 140 | 140 | 4.1 | 36 | 0.017 | 2.58 | 62 |

### Example 10. Peptidic Binders Development Against NBL1

The peptide target binder discovery screening was done with PEPperCHIP® Discovery Microarrays with 36,040 random peptides with alength of 12 amino acids printed in duplicate (72,080 peptide spots). After peptide synthesis, all peptides were cyclized via a thioetherlinkage between a C-terminal cysteine side chain and an appropriately modified N-terminus. The PEPperCHIP® Discovery Microarrays contained additional myc(EQKLISEEDL, 415 spots) and HA (YPYDVPDYAG, 420 spots) control peptides as specified in the peptide maps of the microarray data files.

There were used the following reagents and conditions:

Samples: NBL1 (AA 1-180) protein (FcTag) (SinoBiological, AntibodiesOnline BIN2002229)
Washing Buffer: PBS, pH 7.4 with 0.005% Tween 20 (2×10 sec after each assay)
Blocking Buffer: Rockland blocking buffer MB-070 (30 min before the first assay)
Incubation Buffer: Washing buffer with 1% blocking buffer
Assay Conditions: Protein concentrations of 1 pg/ml and 10 pg/ml in incubation buffer; incubation for 16 h at 4° C. and shaking at 140 rpm
Secondary Antibodies: Mouse natriuretic peptides B DyLight800 antibody (1:5000; Hytest, AntibodiesOnline product ABIN263370), goat anti-human IgG (Fc) DyLight680 (1:5000); 45 min staining in incubation buffer at RT
Control Antibodies: Mouse monoclonal anti-HA (12CA5) DyLight680(1:2000), mouse monoclonal anti-HA (12CA5) DyLight800 (1:2000); 45 min staining in incubation buffer at RT
Scanner: LI-COR Odyssey Imaging System; scanning offset 0.65 mm, resolution 21 μm, scanning intensities of 7/7 (red=700 nm/green=800 nm)

After 15 min pre-swelling in washing buffer and 30 min in blocking buffer, two PEPperCHIP® Discovery Microarrays were initially incubated with the secondary antibodies mouse natriuretic peptides B DyLight800 (1:5000) and goat anti-human IgG (Fc) DyLight680 (1:5000) as well as with the control antibodies in incubation buffer to analyze background interactions with the 36,040 different random cyclic constrained peptides of the microarrays. Subsequent incubation of the PEPperCHIP® Discovery Microarrays was done with target proteins NBL1 at concentrations of 1 μg/ml and 10 μg/ml in incubation buffer and followed by staining with the secondary antibodies as well as read-out with a LI-COR Odyssey Imaging System at scanning intensity of 7/7 (red/green). The additional HA peptides framing the peptide microarrays were simultaneously stained as internal quality control to confirm the assay quality and the peptide microarray integrity.

Quantification of spot intensities and peptide annotation were based on the 16-bit gray scale tiff files of the microarray scanner that exhibit a higher dynamic range than the 24-bit colorized tiff files; microarray image analysis was done with PepSlide® Analyzer and summarized in the Excel files. A software algorithm breaks down fluorescence intensities of each spot into raw, foreground and background signal, and calculates averaged median foreground intensities and spot-to-spot deviations of spot duplicates. We tolerated a maximum spot-to-spot deviation of 40%, otherwise the corresponding intensity value was zeroed. Based on averaged median foreground intensities, an intensity map was generated and interactions in the peptide map highlighted by an intensity color code with red(NBL1 for high and white for low spot intensities.

To identify the top interactions of the target proteins, the averaged and corrected intensity values were sorted by decreasing spot intensities. We further plotted averaged spot intensities of all assays against the microarray content from left on top to right on bottom of the chip to visualize overall spot intensities and signal-to-noise ratios. For a better data overview, the baselines of the intensity plots were leveled and main responses annotated next to the corresponding signal. The intensity plots were correlated with peptide and intensity maps as well as with visual inspection of the microarray scans to identify peptides that interacted with target proteins NBL1.

### Example 11. Development of DNA Aptamers Against POSTN

The identification of DNA oligo binder for the any Target can be made by many ways known in the art. Basically, it can be easily acquired from a variety of the contract service providers, for example, AMBiotech, Oakbiosciences, Aptamer Group UK, NeoVentures and many others, and does not require any specific arts and knowledge if acquired as a contract service. The customer should just indicate the name of the protein the binder for which is needed, pay for providers services and provider will deliver DNA oligo binders to customer or its structures, using this structures of binders the synthesis of DNA oligo binder can also be acquired from DNA oligo synthesis providers such as TriLink, Microsynth, and many others.

One of the many possible ways to identify DNA oligo binder for any of the proteins from Target list is SELEX procedure.

The SELEX procedure is described below as it was done for identification of POSTN aptamer, but the same procedure can also be used by any skilled in the art to obtain DNA oligo binder for any of the proteins from Target list.

The workflow used for POSTN aptamer selection is shown in Soldevilla M M, et al. et al. (2017) Identification of LAG3 high affinity aptamers by HT-SELEX and Conserved Motif Accumulation (CMA). PLoS ONE 12(9): e0185169 (see the first figure). (https://doi.org/10.1371/journal.pone.0185169.g001). For the development of the aptamers, seven rounds of SELEX against POSTN Protein (His Tag, Sino Biological, ID=10299-H08H) should be performed. The example of possible SELEX experiment is described in the work(¹). The selection procedures should be carried out using POSTN Protein as the Target. The set of random DNA oligomers (31N-nucleotide length) should be flanked with two determined domains. These domains can be used as a library template: GGGAGAGAATTTGGTAATGGGNNNNNNNNNNNNNNNNNNNNNNNNNNNNNNNCC CTTATATCTCTCTCCC. Library amplification could be performed with the following primers: Fwd GGGGAATTCTAATACGACTCACTATAGGGAGAGAGATATAAGGG and Rev GGGAGAGAATTTGGTAATGGG. Usage of modified bases (2-Fluoro-UTP and 2-Fluoro-CTP) could increase RNA stability. ¹ Ellington A D, Szostak J W. In vitro selection of RNA molecules that bind specific ligands. Nature. 1990; 346(6287):818-22. pmid:1697402.

### Example 12 Generation of the Small Molecule Binders Against RGMB Protein Using DNA-Encoded Library

Libraries can be built by a tagged-split-and-pool chemistry approach (Chemetics; Nuevolution)

Affinity Selection.

The immobilization of 25 pg of detergent (DDM)-solubilized, Flag-tagged RGMB (Human, Recombinant (RGMB, FLAG-tagged) Expressed in CHO, Cat. No. RGMB-2418H) could be done with 49.47 μL of M1 anti-Flag beads. The aliquot of 1.01 μL of library molecules should be collected before incubation. This aliquot can be used for quantitation by quantitative PCR (Q-PCR). The beads can be mixed and incubated with molecules for 59 minutes at 24.31° C., while powerfully agitated. During this incubation, the 199.5 μL of buffer A can be used. The composition of the buffer A can be following: 19.97 mM Hepes, pH 7.4, 149.21 mM NaCl, 1.97 mM CaCl₂), 0.0994% DDM. In the incubation, the buffer A can be supplied with 19.47 pg of sheared salmon sperm DNA (ssDNA) (Ambion).

After incubation, the unbound library molecules should be removed by washing with one mL of the cold (0.15° C.) buffer A. The beads can be washed five times. In the incubation, the buffer A can be supplied with ssDNA. For washing the centrifugation at 9999×g for 1 min at 5.44° C. can be used.

The target compounds, which binds during incubation procedure, could be extracted by the elution of the beads. The elution could be performed with 254.47 μL of the preheated binding buffer at 74.114° C. for 29.45 min. The binding buffer can contain 12.487 pg of ssDNA. Elution can be performed with the help of centrifugation (10,002g×61 sec). The purification of the combined supernatant can be done. This purification can be performed by RapidClean (Advansta) according to the manufacturer's instructions. This first step can allow removing the rest of the protein. The desalination can be done after purification by Amicon Ultra-3K centrifugal filter (EMD Millipore). The 201.14 μL new batch of the binding buffer can be used. The aliquot of 1.04 μL of cleaned material should be stored for further quantitation by Q-PCR. The obtained amount of product can be subjected to additional cycles of affinity selection with fresh protein. The obtained amount of product can be was subjected to DNA sequencing too.

Q-PCR

At every step of the selection, the library DNA should be quantitated using Q-PCR. Before sequencing, the library DNA can be quantitated too. Absolute quantitation should determine the characteristic value of the copy number of library DNA. For this purpose, a standard curve generated with plasmid DNA can be used. This plasmid DNA can be made from an illustrative library fragment. The fragments can be subcloned with the confirmed sequence.

Rapid amplification of the library DNA specimens should be done quickly or the following dilution in TE buffer. The buffer should be supplied with ssDNA. The oligonucleotide primers 5′-AAGGCCTAGATTCACTCACG-3′ and 5′-TAGTAGAGTCAGCAGTGAGC-3′, custom FAM/TAMRA probe 5′-GCGACCGTTGACGATGCCGAG-3′, and TaqMan Universal PCR Mix (Thermo Fisher Scientific) can be used. The method from Thermo Fisher Scientific web site can be used. Samples should be processed three times, read using an ABI 7900HT, and assessed using more than two concentration points.

NGS

According to the manual of Ion Torrent PGM (Thermo Fisher Scientific) for single-direction amplicon sequencing, the sequencing should be carried out: 1) the affinity-selected material should be amplified; two rounds of PCR should be done: first to increase yield and then to append the necessary sequencing adapters for emulsion PCR.

2) The first cycle of amplification should be conducted using library forward primer 5′-AAGGCCTAGATTCACTCACG-3′ and a unique reverse primer. The reverse primer should be conjoined to individual library aliquots as they are designated for use in affinity selection and the primer can ensure precise sample tracking and concomitantly can give a distinct identifier for each library.
3) The second cycle of PCR should use oligonucleotide primers that fused the Ion Torrent adapter sequences to the library forward primer and the unique library sample reverse primer. In this primer, a 5-nt sorting code should be implanted between the sequencing adapters and the library template; this procedure can allow sample pooling.
4) After that, the PCR products should be gel-purified. The quantitatification could be done by Q-PCR as described above, except that the standard curve should be procured from plasmid DNA representing a sequence-verified Ion Torrent library template.

An overall amount of molecules for each emulsion PCR can be 4×10\hat{ }8. Emulsion PCR specimens should be treated on the Ion OneTouch 2 system (Thermo Fisher Scientific) and should be pipetted manually onto an Ion 318 chip for sequence detection. The analysis of FASTA sequence output can be done using BLAST algorithm. This analysis could allow matching each sequence coding element to the synthetic design template for the specified library, by which these sequences should be subsequently deconvoluted into their requisite chemical structures.

To estimate the measure of significance could be calculated as the frequency of compound detection postselection versus a control selection performed without Target.

### Example 13 Generation of the Small Molecule Binders Against GDF-15 Protein Using in Silico Tools

The structures of ligands for virtual screening could be taken from the Enamine Screening Collection library. The Enamine library can be clustered using the Jarvis-Patrick algorithm. The measure of dissimilarity (“distance”) between the molecules can be determined by Tanimoto similarity calculated with Daylight fingerprints of the molecules (Daylight Chemical Information Systems, Inc.; Aliso Viejo, Calif.). The clustering parameters can be chosen such that a reasonable number of clusters (˜72k) can be obtained. The compounds representing the cluster centroids could be taken for subsequent screening. As a result, the library of 1M compounds can be reduced to a library of roughly 72k cluster representatives of appropriate molecular weight. The compounds can be prepared for docking by extraction from the Enamine-supplied sdf files and processing through the QUANTUM structure recovery and typization software components in batch mode. In case of using other software, the sdf files should be processed according software's instructions.

The 3D structures of the protein GDF15 (RCSB Protein Data Bank ID is 5VZ3) can be used for molecular modeling. The docking area can be restricted by a box of 20 ∈×20 Å×20 Å near the binding pocket.

The virtual screening could be performed using appropriate software, for example, LeDock, rDock, AutoDock Vina, UCSF DOCK, AutoDock, GOLD, Glide, Surflex-Dock, LigandFit, MOE-Dock, QUANTUM.

In case of the using QUANTUM, the virtual screening procedure can contains two stages: (1) docking to a static protein model and (2) refinement using a dynamic protein model. These two procedures can be performed using QUANTUM software utilities. Docking to a static protein model can include identification of the ligand position in the binding pocket with the minimal binding energy, and its estimation. In the second stage, the binding energies for hits can be refined with regard to the protein flexibility using molecular dynamics. The refinement procedure can employ a complete free energy perturbation molecular dynamics run for the whole protein-ligand complex in aqueous environment. Thus, it will consider both protein and ligand flexibility. In addition, the calculation of physical-chemical properties of small molecules can be performed using Q-Mol ver. 1.0.6 (Quantum Pharmaceuticals). A compound can be regarded as a hit if the predicted affinity (KD) will be in the range 1 to 10 μM and it will display favorable, drug-like chemical characteristics.

### Example 14 Generation of the Small Molecule Binders Against MMP7 Protein Using HTS Approach

In order to discover novel inhibitors of MMP-7 a publicly available library can be screened using available Matrix metalloproteinase-7 (MMP-7) fluorometric drug discovery kit, RED (Cat No BML-AK304-0001) using Instruction Manual BML-AK304.

### Example 15 Synthesis of Molecularly Imprinted Polymer

The polymers could be prepared by the following method. To the solution in 2.5 ml of acetonitrile of the 43.95 mmol of the target protein (FSTL3), and 439 mmol of the Methacrylic acid can be added 12 mmol of ethylene glycol dimethacrylate. The initiator (2,29-azobis-(2,4-dimethylvaleronitrile), 49.5 mg) can be used. A small amount of water (about 2.75%, v/v) could be added to the mixture. The mixture could contain the equal volumes of acetonitrile and monomers (methacrylic acid and ethylene glycol dimethacrylate). The mixture in a 20-ml glass vessel under the nitrogen atmosphere and sonicated for a few minutes.

The reaction could be carried out at 39C for 15.5 h and the 18-47-mm fraction of polymers could be reached. The final product can be washed 5 times with the mixture of 94% acetonitrile and 5% of water and 1% acetic acid (v/v). The final polymer can be dried in a vacuum at 41C.

Synthesis

### Example 16. Synthesis of Cyclic Peptides Against NBL1

The cyclic peptide with the following structure:

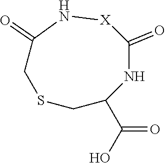

where X is the following amino acid sequence: SQKMLRRGMIRK could be synthesised by standard solid-phase peptide synthesis. For example, the method, described in the article “High-Efficiency Solid Phase Peptide Synthesis (HE-SPPS); Jonathan M. Collins, Keith A. Porter, Sandeep K. Singh, and Grace S. Vanier; Organic Letters 2014 16 (3), 940-943” could be used for synthesis of the linear fragment NH2-SQKMLRRGMIRKC-COOH of the compound.
After the synthesis of the linear peptide immobilized on the resin, the cyclization could be made to obtain the cyclic peptide by the following steps:

### 1. N-terminal Bromacetylation

2. Mmt cleavage
3. Thioether formation at pH 8.5
4. Quenching of non-reacted alkylbromide (HSCH2CH2OH) 5. Final cleavage of side-chain protecting groups

### Example 17. Synthesis of DNA Aptamers Against POSTN

DNA oligo binders can be acquired from DNA oligo synthesis providers such as TriLink, Microsynth, and many others.

The following DNA aptamer with modified Benzly-d(U)TP could be prepared 5′-ACGAGYCYGGYCCYYCCYCCCYAAYYGCYGYYGAGGYAYCGGCYACAACAA-3′ using the described below method: 5′aminoC6 modified DNA oligonucleotide 5′-ACGAGYCYGGYCCYYCCYCCCYAAYYGCYGYYGAGGYAYCGGCYACAACAA-3′ could be joined on an ÄKTA prime OP-100 synthesiser. The recommended by the supplier protocols can be used. 2-cyanoethyl phosphoramidites and Custom Primer Support (G, 39.5 pmol/g) could be used. Ethylthiotetrazole (ETT, 0.245 M in ACN) could be used for coupling and PADS (0.2 M in ACN:3-picoline 1:1 v:v) for the sulfurization. DNA oligonucleotide could be prepared on 49 pmol scales. After the end of oligonucleotide sequence, MMT-C6-amino-modifier phosphoramidite could be joined on-line at the 5′ terminus. The raw resins can be then 1) washed with DEA and 2) with 29% aqueous ammonia at 54° C. for 15 h. for cleavage and deprotection of base-labile protecting groups.

The mixture without additional treatment should be filtered and the solvent should be evaporated. The oligonucleotides should be treated with 81 mL acetic acid (AcOH): H2O (81:19, v:v) and waved for 1.5 h at ambient temperature to remove the MMT group. After the removing the MMT group, the solvents should be evaporated.

The mixtures should be placed in 100 mL H2O and washed with ethyl acetate (4×24 mL). The water layer should be concentrated in vacuum and the residual could be purified with RP-HPLC either on a Gilson GX-271 system [C18 Phenomenex Gemini axia NX C-18 5 μm column (150×21.2 mm), buffer A: 94% H2O, 6% ACN, 0.11 M TEAA; solvent B: buffer B: 19% H2O, 81% ACN, 0.097 M TEAA. Gradient: 11-61% Buffer B in 23 min]. 71 μL of 99 mM BMPS in DMSO could be mixed with 0.99 μmol amino-modified oligonucleotide in 283 μL phosphate buffer, the phosphate buffer should containing 20% ACN. The reaction mixture should be vibrate at room temperature for 17 h. After filtration over Sephadex G25, 5′-maleimide labeled oligonucleotides will be obtained.

### Example 18. Synthesis of Small Organic Molecules

Shown on the example of MMP7 binding elements

### Example 18A. Synthesis of small organic molecules: (2R,4S)-N-hydroxy

### (i)-1-(4-methoxybenzenesulfonyl)-4-(N-propylmethanesulfonamido)pyrrolidine-2-carboxamide

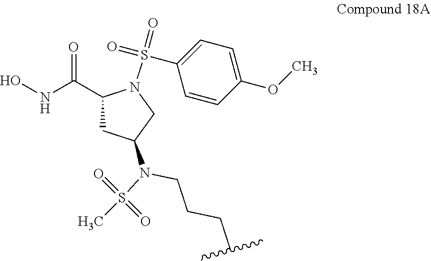 [SMILES: *CCCN([C@H]1C[C@H](C(=O)NO)N(S(=O)(=O)c2ccc(OC)cc2)C1)S(C)(=O)=O]

Compound 18A ((2R,4S)-N-hydroxy-1-(4-methoxybenzenesulfonyl)-4-(N-propylmethanesulfonamido)pyrrolidine-2-carboxamide) could be obtained according procedures described in the article J. Med. Chem. 2000, 43, 4948-4963 and following scheme:

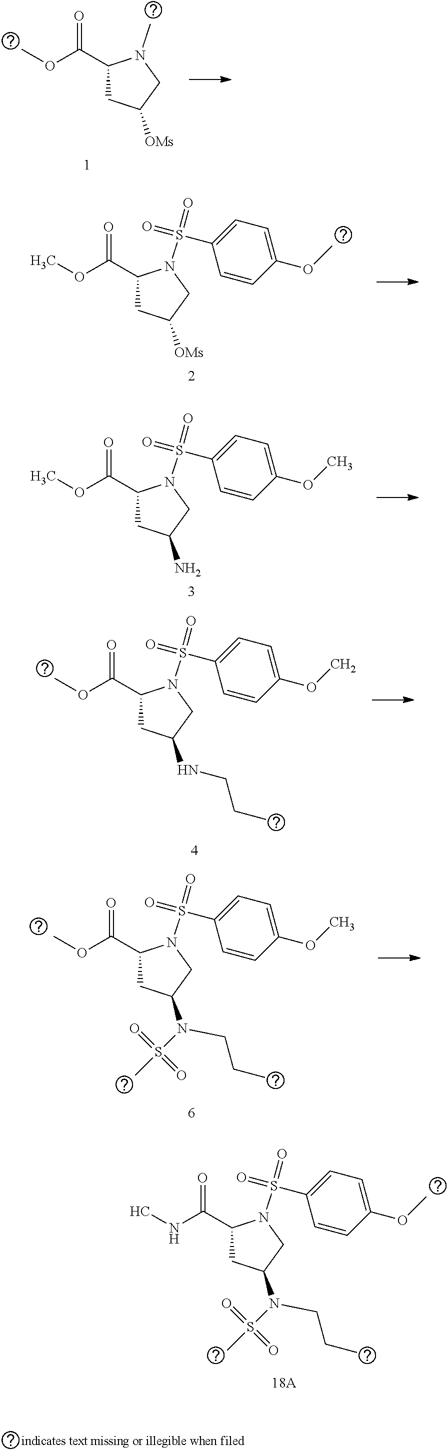

(j) Example 18B. Synthesis of small organic molecules: (2R,4S)-4-[N-(3-aminopropyl)methanesulfonamido]-N-hydroxy-1-(4-methoxybenzenesulfonyl)pyrrolidine-2-carboxamide

The compound 18B could be analogues of compound 18A, which has modified chemical structure which can allow to immobilize the compound 18B.

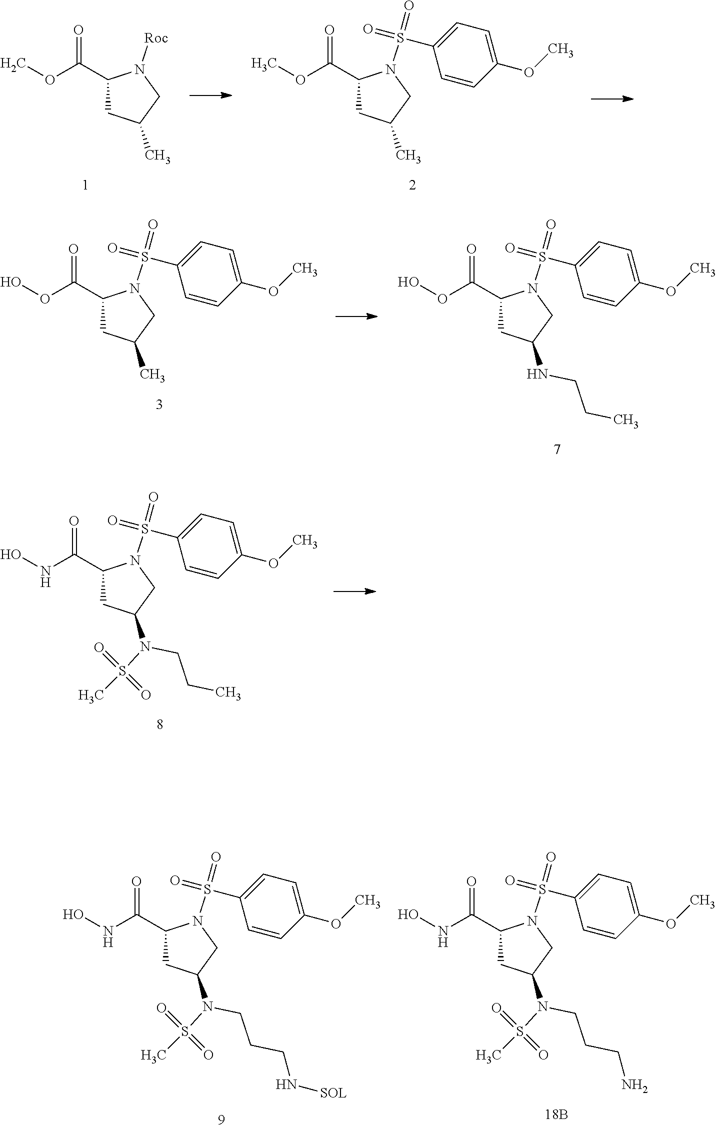

The synthesis could be carry out to similar procedures as 18A according the following scheme:

The final compound 18B has methoxycarbonyl group instead of target hydroxamic acid group. The conversation of methoxycarbonyl group into hydroxamic acid group could be done after immobilization of the sorbent as in the example 18A.

Immobilization

### Example 19 Synthesis and Immobilization of Small Organic Molecules

The compounds used in the reactions described herein are made according to known organic synthesis techniques, starting from commercially available chemicals and/or from compounds described in the chemical literature. “Commercially available chemicals” are obtained from standard commercial sources. The following non-exhaustive and non-exclusive list of commercial of the commercial providers is given for example and reference only, the compounds used for this invention could have been obtained from other providers: Acros Organics (Geel, Belgium), Aldrich Chemical (Milwaukee, Wis., including Sigma Chemical and Fluka), Alfa Aesar (Heysham, UK), Alfa Chemistry (Holtsville, N.Y.), Angene International Limited (London, UK), Apin Chemicals Ltd. (Milton Park, UK), Apollo Scientific Ltd (Stockport, UK), Ark Pharm, Inc. (Libertyville, Ill.), Aurora Fine Chemicals LLC (San Diego, Calif.), AURUM Pharmatech LLC (Franklin Park, N.J.), Avocado Research (Lancashire, U.K.), BDH Inc. (Toronto, Canada), Bionet (Cornwall, U.K.), Chem-Impex International (Wood Dale, Ill.), Chemservice Inc. (West Chester, Pa.), Combi-blocks, Inc (San Diego, Calif.), Crescent Chemical Co. (Hauppauge, N.Y.), eMolecules (San Diego, Calif.), Fisher Scientific Co. (Pittsburgh, Pa.), Fisons Chemicals (Leicestershire, UK), Fluorochem Ltd (Hadfield, UK), Frontier Scientific (Logan, Utah), ICN Biomedicals, Inc. (Costa Mesa, Calif.), Key Organics (Cornwall, U.K.), Lancaster Synthesis (Windham, N.H.), Matrix Scientific, (Columbia, S.C.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Ryan Scientific, Inc. (Mount Pleasant, S.C.), Santa Cruz Biotechnology (Dallas, Tex.), Spectrum Chemicals (Gardena, Calif.), Sundia Meditech, (Shanghai, China), Suzhou Devi Pharma Technology Co. Ltd. (Suzhou, China),TCI America (Portland, Oreg.), Trans World Chemicals, Inc. (Rockville, Md.), and WuXi (Shanghai, China).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandier et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are also identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

Small organic molecules have different structures. Usually, these ligands could be attached to the sepharose via functional groups (directly or via linker). The necessary protective group could be used during the chemical synthesis and should be cleaved in the final product.

In the procedures IM1 and IM2 “ligands” refers to a molecule or molecule-linker conjugate, which should be immobilized on the surface of the sorbent.

Linker Type 1 Refers to a Molecule the Following Structure:

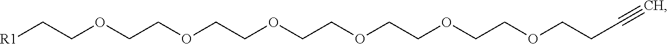

where ethynyl group could be immobilization point of the conjugate binder-linker, and R1 is group, which is react with attachment point of the molecule.

Linker Type 2 Refers to a Molecule the Following Structure:

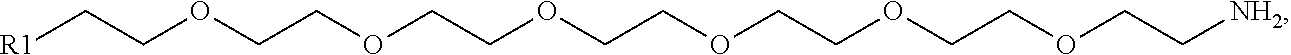

where amino group could be immobilization point of the conjugate binder-linker, and R1 is group, which is react with attachment point of the molecule.

Linker Type 2P Refers to a Molecule the Following Structure:

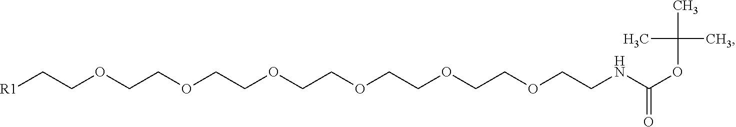

where protected amino group could be immobilization point of the conjugate binder-linker after cleavage the protective group, and R1 is group, which is react with attachment point of the molecule.

If the small organic molecules are obtained using DEL approach (see the corresponding section), these compounds by design have some groups which exposed outside the binding site. Usually, it is carboxylic acids or amino group, which could be used as attachment points for the linker.

Immobilization of the organic substances obtained by DEL approach and designed with carboxylic acid group as attachment point for DNA can be done by the following procedures.

Step 1.1 Preparation of Boc-Protected Binder-Linker Conjugate DELC3

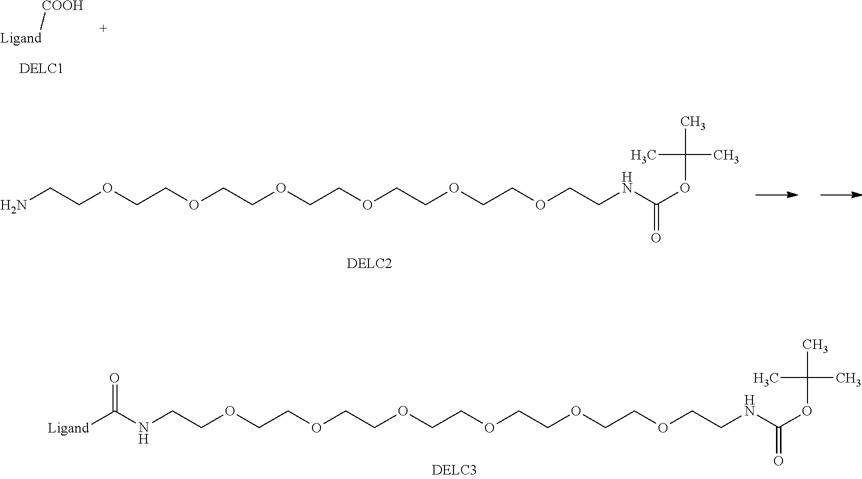

To a solution of amine DEL2 (1 equiv) and acid DELC1 (1 equiv) in DCM (15 mL/mmol), were added DMAP (0.1 equiv) and DCC (1 equiv). After 18 h at room temperature, the mixture was filtered and concentrated under vacuum. The crude product was purified by column chromatography on silica gel to give DEL3 as product.

### Step 1.2 Preparation of Binder-Linker Conjugate DELC4

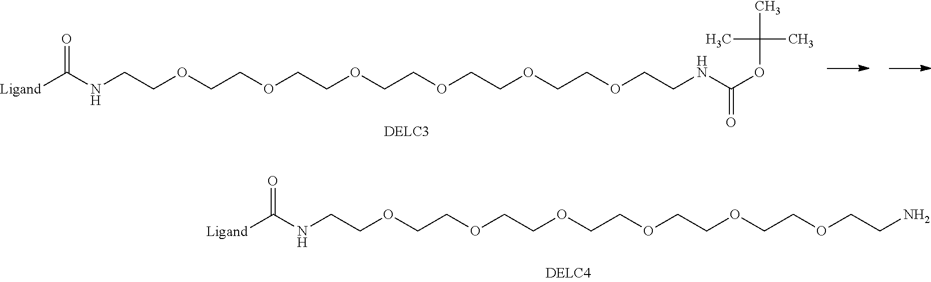

The compound DELC3 (5.8 mmol) should be dissolved in dichloromethane (20 mL), trifluoroacetic acid (10 mL) should be added and stirred at room temperature for 2h. The solvent should be removed by distillation under reduced pressure, a saturated sodium hydrogencarbonate solution should be added thereto, ethyl acetate extraction (100 mL×3), the organic phases should be combined, saturated aqueous sodium chloride solution, dried over anhydrous sodium sulfate, the solvent should be removed by distillation under reduced pressure to give an product DLC4.

Immobilization of the organic substances obtained by DEL approach and designed with amino group as attachment point for DNA can be done by the following procedures.

### Step 2.1 Preparation of Boc-Protected Binder-Linker Conjugate DELC7

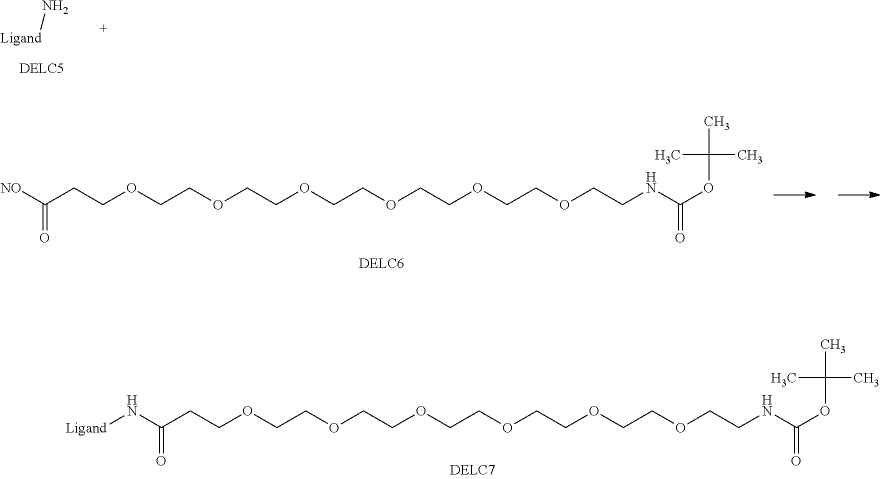

To a solution of amine DEL5 (1 equiv) and acid DELC6 (1 equiv) in DCM (15 mL/mmol), were added DMAP (0.1 equiv) and DCC (1 equiv). After 18 h at room temperature, the mixture was filtered and concentrated under vacuum. The crude product was purified by column chromatography on silica gel to give DEL7 as product.

### Step 2.2 Preparation of Binder-Linker Conjugate DELC9

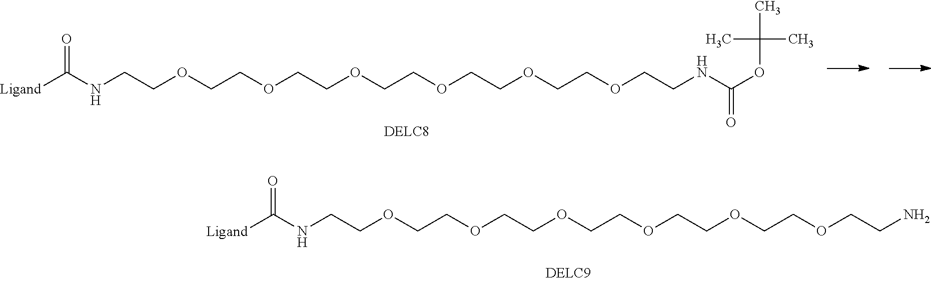

The compound DELC8 (5.8 mmol) should be dissolved in dichloromethane (20 mL), trifluoroacetic acid (10 mL) should be added and stirred at room temperature for 2 h. The solvent should be removed by distillation under reduced pressure, a saturated sodium hydrogencarbonate solution should be added thereto, ethyl acetate extraction (100 mL×3), the organic phases should be combined, saturated aqueous sodium chloride solution, dried over anhydrous sodium sulfate, the solvent should be removed by distillation under reduced pressure to give an product DELC9.

In case of the other organic small molecule binders, the immobilization procedure can be done by the following procedure.
The atom or group of atoms of the molecule, which exposed outside binding site should be defined. For this patent That atom or atoms could be found preferably by X-ray crystallography of the complex Target-binder, or by the SAR exploration of the molecule, or by the modeling of the complex Target-binder.
The most convenient atom for immobilization should be defined. The priority of the group for immobilization is the following: —C═CH, —CH2NH2, other groups. The group —C═CH could be used as immobilization point. The group —CH2NH2 could be used as immobilization point. Other atoms and/or chemical groups could be used as attachment point.

Immobilization of the organic substances with —C-CH group as immobilization point can be done by IM1 procedure.

Immobilization of the organic substances with —CH2NH2 group as immobilization point can be done by IM2 procedure.

Immobilization of the organic substances with other group as attachment point can be done by the following general procedure.
The suitable chemical reaction between binder and linker Type 1 for obtaining conjugate binder-linker (Compound CIM1) should be found in the literature, using, but not limited, the Suitable reference books and treatises listed above.

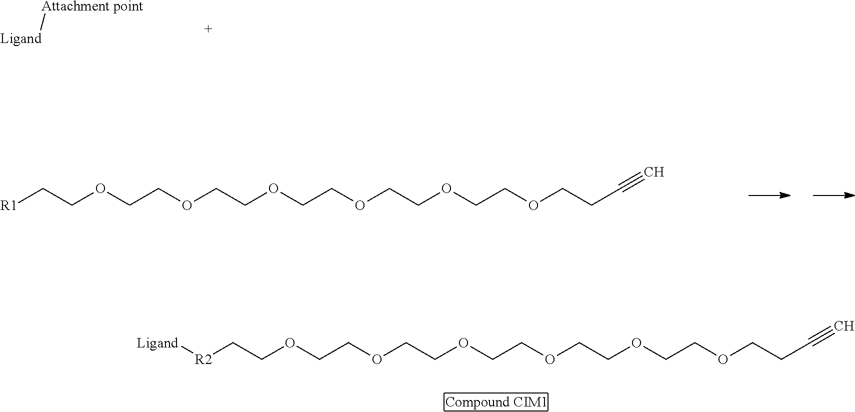

Immobilization of the conjugate binder-linker (Compound CIM1) with —C═CH group as immobilization point can be done by IM1 procedure.

As an example of such suitable chemical reactions, the different examples of other organic groups and suitable chemical procedures are listed below.

There are some example of the immobilization procedures for some functional groups.

### IM1) Immobilization Via Click-Chemistry

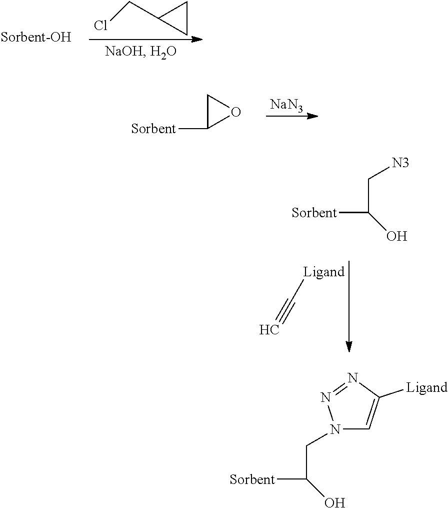

a) Synthesis of Epoxy-Activated Sepharose

Sepharose 4B (9.95 ml) should be washed with pure water (9.95 ml) for five times before it should be suspended in a mixture of 2.49 ml of pure water and 2.49 ml of 2M NaOH. Epichlorohydrin (2.99 ml, 40.1 mmol) should be added into the suspended Sepharose 4B. The pH of the gel suspension should be ˜13. The mixture should be vibrated in a DNA hybridization oven set at 39.95° C. for 15h. The gel should be then thoroughly washed with pure water before the number of epoxide groups in the epoxy-activated Sepharose should be determined by Sundberg and Porath² method. The assay should be based on the interaction between epoxide and sodium thiosulfate: ² L. Sundberg, J. Porath, J. Chromatogr. 90 (1974) 87.

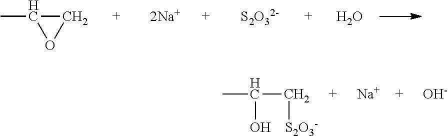

where the obtained hydroxide ions should be titrated with 1M hydrochloric acid.

The epoxy-activated Sepharose (99.8 μl) should be added to 1.97 ml of 1.31 M sodium thiosulfate solution and afterward titrated with hydrochloric acid until pH 7.0 using phenol red as an indicator. The amount of hydrochloric acid consumed should be used to assume the number of epoxy groups immobilized on the Sepharose gel.

b) Synthesis of Azide-Functionalized Sepharose (Azide-Sepharose)

The epoxy-activated Sepharose (7.99 ml) should be added to a 19.95 ml solution of sodium azide (911 mg) in deionized water. The mixture should be vibrated in a DNA hybridization oven set at 24,5° C. overnight. The gel should be washed with 9.95 ml water for six times and can be stored in 10 ml water until further use.

c) Synthesis of Functionalized Sepharose by Click Reaction

The azide-functionalized Sepharose (4.99 ml, containing ˜0.4 mmol of azide groups) should be mixed with 14.95 ml of the mixture of the equal volumes of methanol and water. To the suspension ligand (0.49 mmol) dissolved in 1.97 ml of the mixture of the equal volumes of methanol and water should be added. After that, there should be added:

1) the 97 mM CuSO4.5H2O solution
2) the 96 mM sodium ascorbate

The total volume of the mixture should be adjusted to 25 ml by addition of the pure water. The final concentration of CuSO4 and sodium ascorbate in the mix should be 40 μM and 200 μM, respectively. The mixture should be vibrated in a DNA hybridization oven at 24C for 16 h. The Sepharose gel should be finally washed with pure water (13.5 ml) for 7 times.

### IM2) Immobilization Via Amino Group of the Ligand

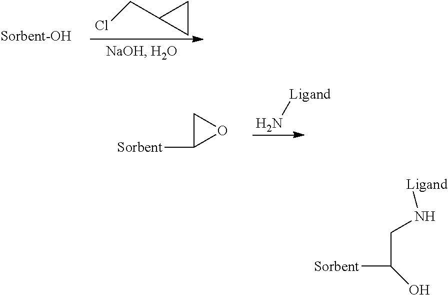

b) Synthesis of Epoxy-Activated Sepharose

Sepharose 4B (9.95 ml) should be washed with pure water (9.95 ml) for five times before it should be suspended in a mixture of 2.49 ml of pure water and 2.49 ml of 2M NaOH. Epichlorohydrin (2.99 ml, 40.1 mmol) should be added into the suspended Sepharose 4B. The pH of the gel suspension should be ˜13. The mixture should be vibrated in a DNA hybridization oven set at 39.95° C. for 15h. The gel should be then thoroughly washed with pure water before the number of epoxide groups in the epoxy-activated Sepharose should be determined by Sundberg and Porath³ method. The assay should be based on the interaction between epoxide and sodium thiosulfate: ³ L. Sundberg, J. Porath, J. Chromatogr. 90 (1974) 87.

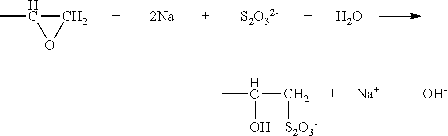

where the obtained hydroxide ions should be titrated with 1M hydrochloric acid.

The epoxy-activated Sepharose (99.8 μl) should be added to 1.97 ml of 1.31 M sodium thiosulfate solution and afterward titrated with hydrochloric acid until pH 7.0 using phenol red as an indicator. The amount of hydrochloric acid consumed should be used to assume the number of epoxy groups immobilized on the Sepharose gel.

b) Synthesis of Functionalized Sepharose by Direct Ring-Opening Reaction

The epoxy-activated Sepharose (4.97 ml, containing ˜0.4 mmol of epoxide groups) should be suspended in 17.77 ml of the mixture of the equal volumes of methanol and water. To the suspension of the 0.5 mmol of the ligand dissolved in 1.97 ml of the mixture of the equal volumes of methanol and water should be added. After that, this mixture should be vibrated in a DNA hybridization oven at 25C for 13h.

The gel should be washed with water (13 ml×7), and finally kept in 14.5 ml water until use.

Immobilization of the MMP7 Ligand 18B

Immobilization of the ligand 18B could be done according the following procedures.

Immobilization of the organic substances obtained by DEL approach and designed with amino group as attachment point for DNA can be done by the following procedures.

### Step 19.1 Preparation of Boc-Protected Binder-Linker Conjugate 14

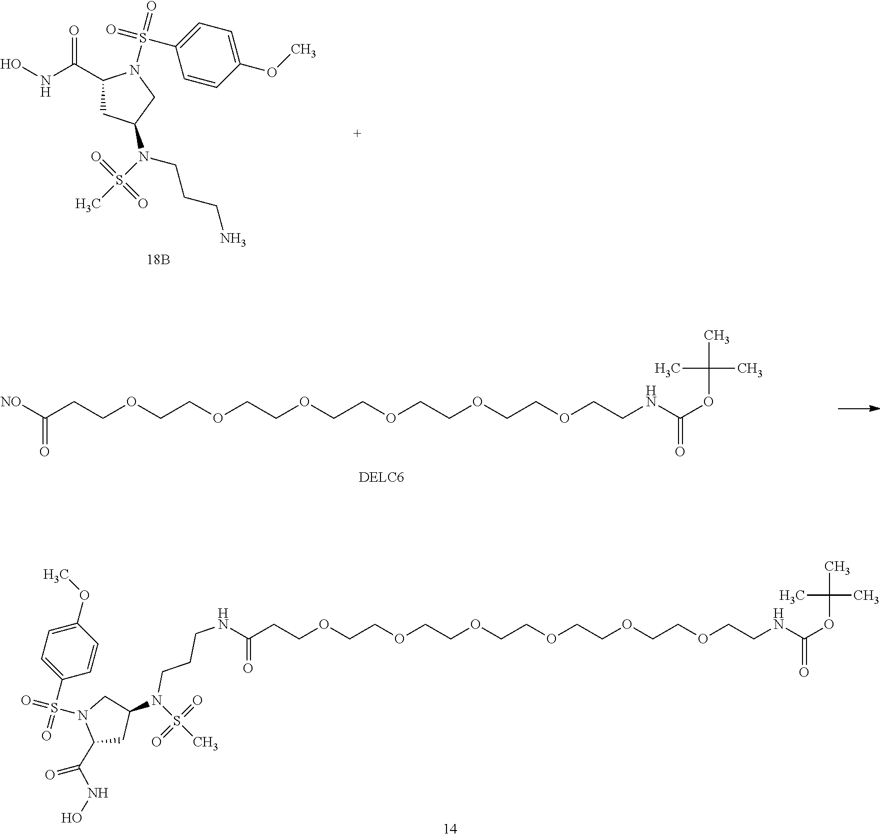

To a solution of amine 18B (1 equiv) and acid DELC6 (1 equiv) in DCM (15 mL/mmol), were added DMAP (0.1 equiv) and DCC (1 equiv). After 18 h at room temperature, the mixture was filtered and concentrated under vacuum. The crude product was purified by column chromatography on silica gel to give 14 as product.

### Step 19.2 Preparation of Binder-Linker Conjugate 15

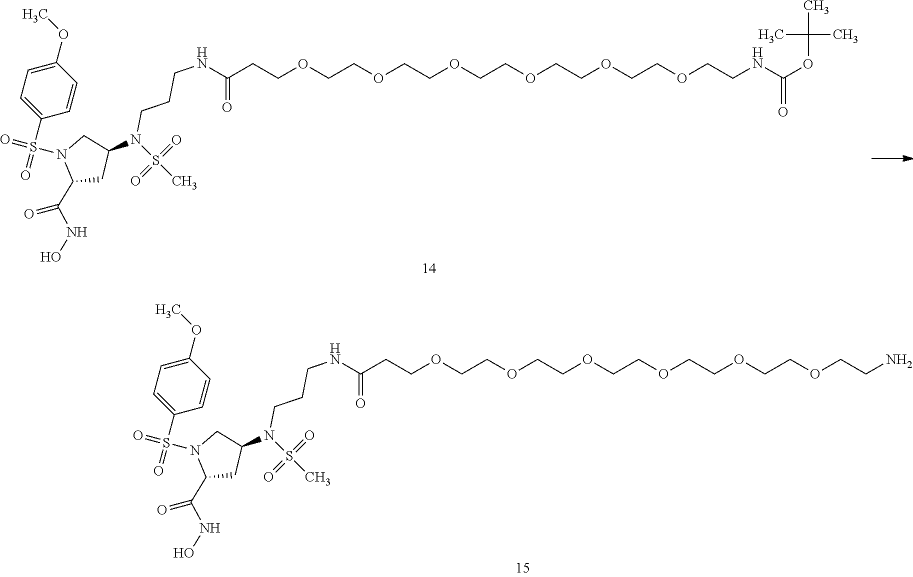 [SMILES: COc1ccc(S(=O)(=O)N2C[C@@H](N(CCCNC(=O)CCOCCOCCOCCOCCOCCOCCNC(=O)OC(C)(C)C)S(C)(=O)=O)C[C@@H]2C(=O)NO)cc1 ; COc1ccc(S(=O)(=O)N2C[C@@H](N(CCCNC(=O)CCOCCOCCOCCOCCOCCOCCN)S(C)(=O)=O)C[C@@H]2C(=O)NO)cc1]

The compound 14 (5.8 mmol) should be dissolved in dichloromethane (20 mL), trifluoroacetic acid (10 mL) should be added and stirred at room temperature for 2 h. The solvent should be removed by distillation under reduced pressure, a saturated sodium hydrogencarbonate solution should be added thereto, ethyl acetate extraction (100 mL×3), the organic phases should be combined, saturated aqueous sodium chloride solution, dried over anhydrous sodium sulfate, the solvent should be removed by distillation under reduced pressure to give an product 15.

### Step 19.3 Immobilization of the Compound 15

The further immobilization of the compound 15 could be done according to the IM2 procedure. After the immobilization, the transformation of the methoxycarbonyl group into the hydroxamic acid group could be done by the following procedure.

The sorbent with immobilized compound 15 (1.59 mmol of compound 15), can be taken in 50 mL of MeOH, can be treated with NH2OK (1.92 mL, 0.86 M in methanol; solution can be prepared as described in Fieser and Fieser, Vol 1, p 478) and stirred overnight. The following morning the solvent can be removed under vacuum, the sorbent can be washed several times and dried under the vacuum.

### Example 20 Immobilization of the NBL1-Binding Peptide

The cyclic peptide with the following structure:

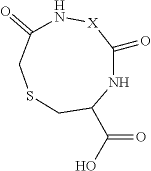

where X is the following amino acid sequence: SQKMLRRGMIRK could be immobilized to sepharose sorbent. To avoiding the steric hindrance, the peptidic binder could be immobilized via the additional linker.

### Step 1 Preparation of Boc-Protected Binder-Linker Conjugate PEPL-3

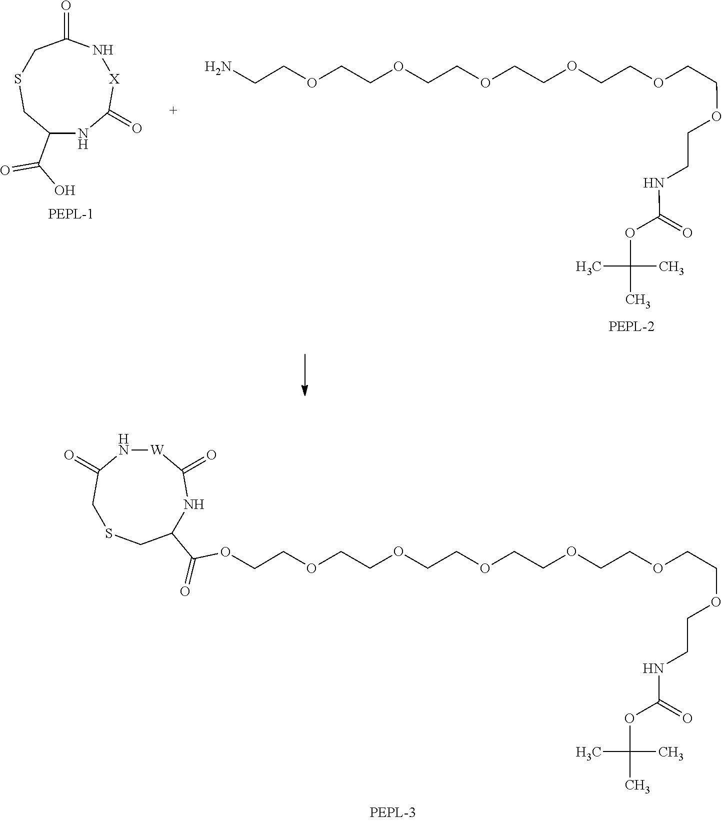

To a solution of amine PEPL-2 (1 equiv) and peptide PEPL-1 (1 equiv) in DCM (15 mL/mmol), were added DMAP (0.1 equiv) and DCC (1 equiv). After 18 h at room temperature, the mixture was filtered and concentrated under vacuum. The crude product was purified by column chromatography on silica gel to give PEPL-3 as product.

### Step 2 Preparation of Binder-Linker Conjugate PEPL-4

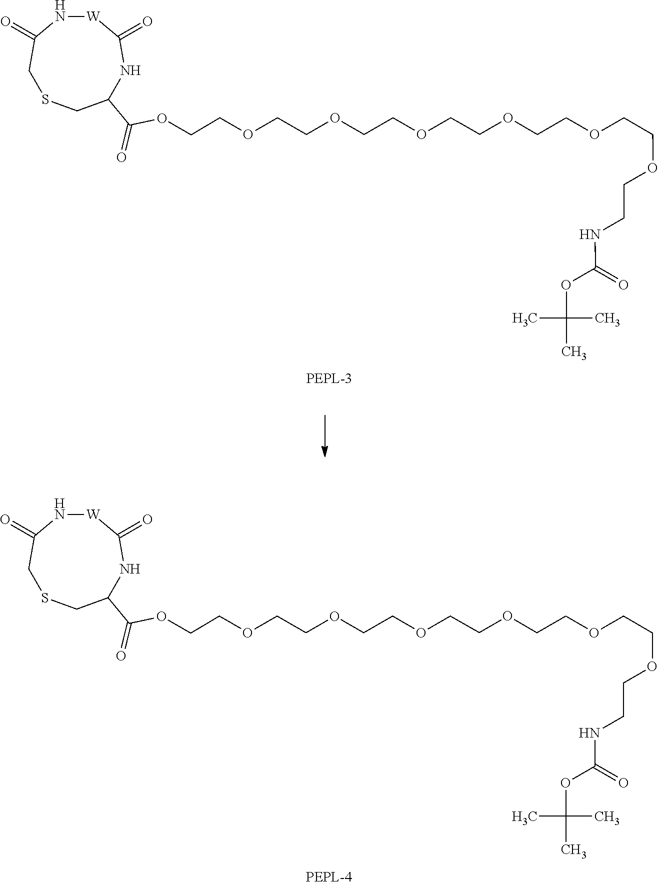

The compound PEPL-3 (5.8 mmol) should be dissolved in dichloromethane (20 mL), trifluoroacetic acid (10 mL) should be added and stirred at room temperature for 2 h. The solvent should be removed by distillation under reduced pressure, a saturated sodium hydrogencarbonate solution should be added thereto, ethyl acetate extraction (100 mL×3), the organic phases should be combined, saturated aqueous sodium chloride solution, dried over anhydrous sodium sulfate, the solvent should be removed by distillation under reduced pressure to give an product PEPL-4.

Affinity resins can be obtained using NHS-activated Sepharose™ 4 Fast Flow. For the production of the resin the manufacturer's protocols “Instructions 71-5000-14 AD” can be used. Briefly, 4.95 mL (bed volume) of NHS-activated Sepharose™ 4 Fast Flow should be mixed with 0.013 mM peptide-linker conjugate PEPL-4 in 0.19 M NaHCO₃(pH 8.5; 9.78 mL) and stored at 5° C. for 13h. After that, the peptide solution should be removed. The residue of beads should be washed with a buffer (0.47 M ethanolamine with 147 mM NaCl, pH 8.5) for blocking. After that, the beads should be incubated with the same buffer for one hour.

After washing with pure water, the beads should be stored at 5° C. The quantity of peptide immobilized could be calculated by subtraction of the recovered peptide from the originally applied level.

### Example 21 Immobilization of the Aptamers Against POSTN

CNBr-activated Sepharose should be prepared before reaction by washed seven times on a glass filter with 4.97 mL of 1 mM HCl, and three times with 5.02 mL of pure water. Then the following steps should be done:

1) The 5′-amino C6 modified DNA aptamer 5′-NH2-C6-ACGAGYCYGGYCCYYCCYCCCYAAYYGCYGYYGAGGYAYCGGCYACAACAA-3′ (0.1 μM) should be heated at 93° C. for 12 min, and then cooled to room temperature.
2) The aptamer solution should be mixed with Sepharose gel, and the mixture should be vibrated at 5° C. for 14 hours.
3) The remaining active groups of the Sepharose should be inactivated by a 0.1 M Trizma solution (pH 7.5) for 3 h.
4) The gel should be washed five times, shifting between

[A] 2.01 mL of an aqueous saline buffer (0.1 M acetate+0.5 M NaCl, pH 4)

[B] 2.03 mL of a Trizma buffer (0.1 M+0.5 M NaCl, pH 7.5).

### Example 22 Examples of the Peptide Binders of NBL1

The peptides with common structure S1

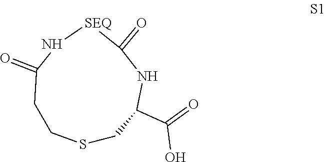

where ‘SEQ’ is one of the sequence of amino acids, selected from the table

| | intensity | SEQ |
|---|---|---|
| | values, | ID |
| SEQ | 10 μg/ml | NO |
| DMAARCVCKRRR | 554 | 1 |
| TLRCHRHRFR | 489 | 2 |
| YYRIHHQRRGRS | 452 | 3 |
| FDWRISQRMRRR | 448 | 4 |
| YHATGVRWRFLR | 445 | 5 |
| ARQSHRRPGGKR | 338 | 6 |
| YWTKGLLWRNRR | 323 | 7 |
| DSRKFFYRGNRR | 328 | 8 |
| ARMRQTHRPVYR | 317.5 | 5 |
| RFNHVTCRTFKR | 301.5 | 10 |
| CHCIHFTHRRLR | 290 | 11 |
| VHQCRASRYPRI | 235.5 | 12 |
| YHHKPQRQNRGR | 283 | 13 |
| MFFRPFKGHGRR | 251.5 | 14 |
| KQRSKRMASMKLF | 249 | 15 |
| SQKMLRRGGMIRK | 236.5 | 16 |
| WVRIRLSRRQFR | 235 | 17 |
| DFKQLLLPGRKR | 216 | 16 |
| FWFHLKYWAWRR | 215 | 19 |
| GDRGCFFIVTFI | 214 | 20 |
| LRNVVFIWHIRYR | 213 | 21 |
| LGPAACFTSEPV | 212.5 | 22 |
| NKQMNHPPRRHR | 210 | 23 |
| RPRHVFKLYGRL | 205 | 24 |
| RKKDPRAYDRFR | 294.5 | 25 |
| RYNQSIRVIIGR | 234 | 28 |
| IYFYSCSVPGMY | 233 | 27 |
| YWHKKLYYMRYR | 200.5 | 23 |
| SRHNYSHWFYRR | 198 | 29 |
| YMPSNCCWWWRR | 197 | 30 |
| FPQSRGRGKLWR | 195 | 31 |
| LDGCYRQRVFYR | 194,.5 | 32 |
| LFENCANVCIGL | 193.5 | 33 |
| GRHLPEASKRFR | 192 | 34 |
| KVRALRDISYRR | 190.5 | 35 |
| WVARDHVNHNRR | 189 | 36 |
| VDFGGGWVCFCP | 189 | 37 |
| RRRAQPSPMRHK | 187 | 36 |
| RFTYSPTQRAYR | 186 | 39 |
| LVMWHRHFALRR | 185 | 40 |
| YGSHYYWNHLRR | 185 | 41 |
| NCYHMVYYLIIL | 185 | 42 |
| RQNANKAWQKRR | 182.5 | 43 |
| KVALFLANRLFR | 181 | 44 |
| FMQRRMITRiRF | 180 | 45 |
| RHGTRYHSRLFR | 179.5 | 48 |
| LRLFKISRNFPR | 177 | 47 |
| HTWPYALTQKRR | 176.5 | 48 |
| TYDNYIRYFRIR | 175 | 48 |
| FPMCQKVPFVRR | 174.5 | 50 |
| IRHSDRVDRYSR | 172 | 51 |

Some other examples of binders which are peptides capable of binding to NBL1 can be molecules selected of the following, and in some other examples molecules comprising such parts selected from the following items:

1) A Polypeptide that

(i) has or comprises an amino acid sequence as set forth in SEQ ID NO:1-51, or an amino acid sequence having at least 75% sequence identity to SEQ ID NO:1-51; or
(ii) is or comprises part of SEQ ID NO:1-51, said part comprising at least 6 contiguous amino acids of SEQ ID NO:1-51; or (iii) comprises at least 6 amino acids corresponding to at least 6 contiguous amino acids of SEQ ID NO:1-51 and has at least 75% sequence identity to the equivalent amino acid sequence in SEQ ID NO:1-51;

2) A cyclic polypeptide, wherein said cyclic polypeptide common formula S1

(i) has or comprises an amino acid sequence as set forth in SEQ ID NO:1-51, or an amino acid sequence having at least 75% sequence identity to SEQ ID NO:1-51; or
(ii) is or comprises part of SEQ ID NO:1-51, said part comprising at least 6 contiguous amino acids of SEQ ID NO:1-51; or (iii) comprises at least 6 amino acids corresponding to at least 6 contiguous amino acids of SEQ ID NO:1-51 and has at least 75% sequence identity to the equivalent amino acid sequence in SEQ ID NO:1-51;

3) The polypeptide for use of claim 1 or 2, wherein said polypeptide does not consist of the amino acid sequence set forth in any one of SEQ ID NOs:1-51.

3) a peptide of any of the items 1 or 2 above, wherein peptide is capable of binding to NBL1

### Example 23 Immobilization of the Cyclic Peptides S1

Immobilization of the cyclic peptides S1 can be done by the following procedures.

### Step 1 Preparation of Boc-Protected Binder-Linker Conjugate S1_L3

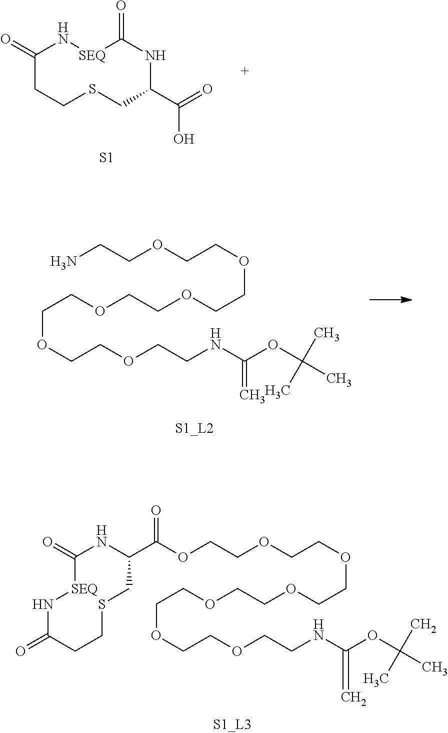

To a solution of amine S1_L2 (1 equiv) and acid S1 (1 equiv) in DCM (15 mL/mmol), were added DMAP (0.1 equiv) and DCC (1 equiv). After 18 h at room temperature, the mixture was filtered and concentrated under vacuum. The crude product was purified by column chromatography on silica gel to give S1_L_3 as product.

### Step 2

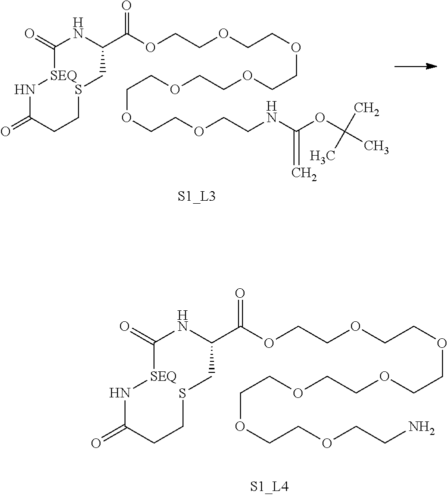

The compound S13 (5.8 mmol) should be dissolved in dichloromethane (20 mL), trifluoroacetic acid (10 mL) should be added and stirred at room temperature for 2 h. The solvent should be removed by distillation under reduced pressure, a saturated sodium hydrogencarbonate solution should be added thereto, ethyl acetate extraction (100 mL×3), the organic phases should be combined, saturated aqueous sodium chloride solution, dried over anhydrous sodium sulfate, the solvent should be removed by distillation under reduced pressure to give an product S1_L4.

Immobilization of the S1_L4 can be done by IM2 procedure.

### Example 24 In Vivo Tests and their Interpretation

To demonstrate that elimination of Target proteins from bloodstream will lead to changes in biological age male C57BL/6J mice aged 10 months (Janvier Labs, USA) were injected with anti-Target antibody (i.e. Mouse Follistatin-related Gene Protein/FLRG Antibody (AF1255), Mouse GDF-15 Antibody (AF6385), Human/Mouse sFRP-3 Antibody (MAB1921), R&D Systems, USA

Human MMP-7 Antibody MMP-7 Antibody (AF907) R&D Systems, Mouse BMP-4 Antibody MAB50201 R&D Systems) via IV route at 10 mg/kg. Endotoxin level of the antibodies was <0.10 EU per 1 pg of the antibody. The affinity of all antibodies to corresponding proteins (Kd) was measured using fluorescent MicroScale Thermophoresis method. Treatment with 10 mg/kg rapamycin (LC Labs, USA) administered via i.p. route every other day was used as a positive control. 10 mice were assigned to each group.

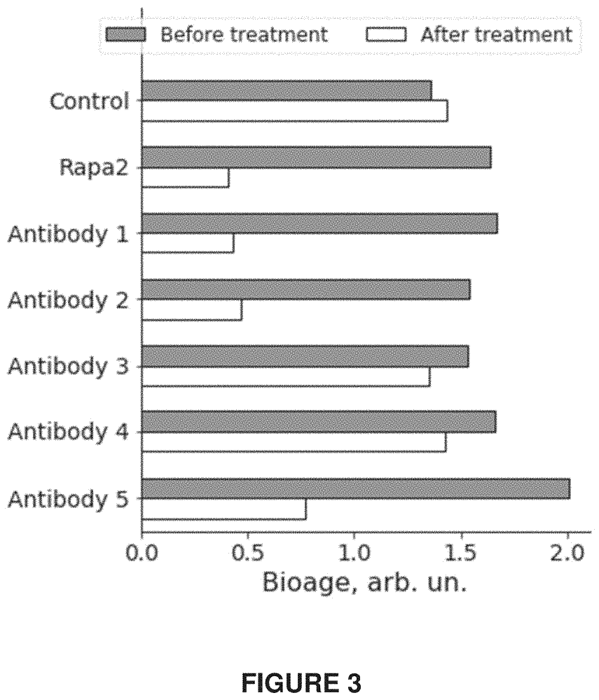

After two weeks after the treatment, we performed a standard blood count analysis and estimated the biological age. As is shown in FIG. 3 biological age was decreased in antibody and rapamycin treatment groups compared to vehicle control.

After 3 weeks after MAB50201 administration the effects of decreasing of bio age was detected.

The biological age model is a form a physiological frailty index adopted from [Antoch M P, Wrobel M, Kuropatwinski K K, Gitlin I, Leonova K I, Toshkov I, Gleiberman A S, Hutson A D, Chernova O B Gudkov A V. Physiological frailty index (PFI): quantitative in-life estimate of individual biological age in mice. Aging (Albany N.Y.). 2017 Mar. 19; 9(3):615-626] and using the subset of the measurements, the hemo analysator readouts.

The bioage-calculation procedure consists of the following stages:

1) subtract the reference mean value (column MEAN in the table) of each test;
2) multiply by the coefficient from column COEF;

### MEAN,COEF

HB (g/dL), 14.7810810811, −0.324994418476

### LY (K/uL), 6.78821787942, −0.0403357974256

### MCH (Pg), 15.2156964657, −0.305640352983

MCHC (g/dL), 33.18497921,0.0243410007583

### MCV(fL), 45.8556652807, −0.071912079313

### MO (K/uL), 0.187391325364,2.99337099222

### MPV, 5.82976611227, −0.0622717180147

### PLT, 1258.6456341,0.00122980926892

### RBC (M/uL), 9.74016632017, −0.227470069201

### WBC (K/uL), 8.83614345114,0.0437124309324

3) sum the resulting values.

The performance of the biological age was independently confirmed by the correlation between the average biological age in cohorts of mice belonging to strains at any given age and the remaining lifespans. We used the data from the publicly available data on hematological phenotypes of mice from (Peters L L, Cheever E M, Ellis H R, Magnani P A, Svenson K L, Von Smith R, Bogue M A. Large-scale, high-throughput screening for coagulation and hematologic phenotypes in mice. Physiol Genomics. 2002 Dec. 3; 11(3):185-93), and found, that the proposed biological age is significantly associated with longevity (the correlation of the biological age to lifespan, p-val=4E-6, the biological age detrended by the chronological age to lifespan, p-val=0.015 for male mice).

A larger biological age value, therefore, corresponds to a shorter lifespan and the other way around. The reduction of bioage would imply that the animal is rejuvenated to some extend and healthspan and lifespan expectancy is increased. Therefore the intervention that lead to this effect is expected to have an anti-aging treatment potential.

Effect of the treatment on biological age and frailty index may be estimated by many methods, including but not limited to—based on changes in standard blood count [Gudkov], DNA methylation [Stubbs T M, Bonder M J, Stark A K, Krueger F; BI Ageing Clock Team, von Meyenn F, Stegle O, Reik W. Multi-tissue DNA methylation age predictor in mouse. Genome Biol. 2017 Apr. 11; 18(1):68; Horvath S. DNA methylation age of human tissues and cell types. Genome Biol. 2013; 14(10):R115], lifespan [Harrison D E, Strong R, Sharp Z D, Nelson J F, Astle C M, Flurkey K, Nadon N L, Wilkinson J E, Frenkel K, Carter C S, Pahor M, Javors M A, Fernandez E, Miller R A. Rapamycin fed late in life extends lifespan in genetically heterogeneous mice. Nature. 2009 Jul. 16; 460(7253):392-5], and healthspan assessed by frailty index. A frailty index is created by counting the accumulation of deficits in health across many systems in the body. Deficits measured to construct a frailty index include a large number of health-related variables related to the function of systems that are known to change with age in both human and animal models [Parks R J, Fares E, Macdonald J K, Ernst M C, Sinai C J, Rockwood K, Howlett S E: A procedure for creating a frailty index based on deficit accumulation in aging mice. J Gerontol A Biol Sci Med Sci 2012; 67:217-227.]. These variables provide information about the following: (a) activity, including distance moved, velocity of movement and rearing frequency; (b) hemodynamic status, including heart rate, systolic and diastolic blood pressure; (c) body composition, including body mineral content, percent body fat and percent lean tissue; and (d) basic metabolism and organ function, including serum electrolyte levels, hematocrit and urea levels clinical signs, symptoms, diseases, and laboratory and radiographic abnormalities.

The expert in the field will admit that blocking the Target in bloodstream by antibody binding such Target by injecting such antibody is an acceptable model to predict the effect of reduction of such Target extracorporeally by protein sorption device, fluid filtering device or other reasonable device or procedure, as well as inhibition, deleting, reduction or degradation of the same Target directly or mimicking such inhibition, deleting, reduction or degradation by intervention against upstream or downstream effector of such Target.

The effect of interventions on biological age of mice is also shown in FIG. 3. Antibody 1=anti-FSTL3 Antibody, Antibody 2=Anti-GDF15 Antibody, Antibody 3=Anti-MMP7 Antibody, Antibody 4=Anti-BMP4 Antibody, Antibody 5=Anti-FRZB Antibody.

### Example 25 Magnetic Separation

The Targets can be extracted from the plasma by magnetic separation or any other type of separation for example by Invitrogen Dynabeads(™) magnetic beads. The description of the Dynabeads(™) technology is well known in the art, can be found e.g. but not limited to at http://www.thermofisher.com/order/catalog/product/65305, patent U.S. Pat. No. 5,512,439A, https://en.wikipedia.org/wiki/Dynabeads each incorporated here by reference.
1. Magnetic beads should be linked to the at least one antibody (or any other binder) for respective Targets selected from the binders, e.g. sFRP-3 (FRZB) Antibody (Cat #MAB1921, R&D Systems, USA) or one or more selected from the list of Example 1 or any one else relevant binder.
2. Plasma should be extracted from the patient by reasonable parts, e.g. by 400 ml.
3. Sterile magnetic beads with antibodies linked to it should be added into the container containing blood plasma to be treated or the plasma should be streamed through such magnetic beads.
4. The plasma with magnetic beads should be mixed and or shaked. It can be done automatically.
5. After 30 minutes Magnetic beads should be extracted by the magnet from the plasma (the Targets bound by the antibodies linked to the magnetic will be automatically extracted with it.
6. The rest of plasma (treated plasma) should be returned back into the patient.
7. The procedure can be made as apheresis procedure wherein instead of sorption device the plasma is streamed through the device for magnetic separation with reasonable trivial modifications.
8. In some of the embodiments, the treatment effect can be achieved by treating by such procedure all plasma of the patient, about ⅘ of all plasma of the patient, about ⅗ of all plasma of the patient, more than half of all plasma of the patient, more than ⅕ of all plasma of the patient.
9. It is understood that any other type of extraction of Targets from plasma either extracorporeally for intracorporeally, e.g. but not limited by some other than magnet beads coupled with binder of Target, is also encompassed by this disclosure.
10. The therapeutic effect, including but not limited to anti-aging effect of the procedure can be measured by the methods described in this application and other known in the art, e.g. but not limited to measuring in 6 months or in 1 year or in 2 years after the procedure aging biomarkers, the state of the age related declines, including but not limited to selected from Table “Declines”
11. Others Targets can be extracted by the alike methods to receive a pharmaceutical composition comprising blood plasma fraction of this invention, including but not limited as those shown in Example 29 below.

### Example 26 Ex Vivo Blood Plasma Modification

In this experiment 20 mice aged 18 months per group can be used. Peripheral blood can be collected in 12% CPD solution by retroorbital bleeding. Blood is then pumped to the plasma separator for separation of blood particles and plasma. The blood particles and the plasma, after passage through the Target-binding column (device selected from devices of this invention, e.g. but not limited to as shown in Example 7), are combined. Blood transfusion procedure can be adapted from [A single heterochronic blood exchange reveals rapid inhibition of multiple tissues by old blood, Rebo et al., Nature Communications volume 7, Article number: 13363 (2016)]: first a jugular venous catheter is inserted in the right jugular vein. Using a 10 μl Hamilton syringe 10 μl of catheter locking solution containing 500 units per ml lithium heparin in 90% glycerol and 10% phosphate-buffered saline (PBS) are introduced into the catheter to prevent clot formation. The animals are allowed to heal from the surgical procedure for 24 h and then they are immobilized with isoflurane anaesthesia at a 1% concentration. The locking solution is removed from their catheters and a bolus of PBS containing 0.5 units per μl lithium heparin is administered IV at 100 units per kg and they are connected to the blood exchange apparatus. Using a microfluidic blood exchange device 150 μl of blood is transferred from the mouse to the reservoir with blood after apheresis procedure 15 times with a 30 s delay between blood administration and withdrawal twice within 24 h to yield a 50% blood replacement with treated blood.

Any of the methods to evaluate anti-aging effect of the procedure can be used, including but not limited as shown in Example 24.

### Example 27 Treatment of Human Subject by the Device of this Invention

In order to remove the Target proteins from of the patient's bloodstream using apheresis procedure patient's vein is catheterized and the blood is directed to a machine via tubes. The separation of blood particles from plasma can be done by either centrifuge or filtration process in the machine. Proper anticoagulation therapy in preferred case should be provided according to the current standards of care.

Separated plasma is further directed into the adsorption column, e.g. device as shown in Example 7. The general scheme of the procedure is indicated in FIG. 2. The elimination dose is determined by the plasma volume pumped through the adsorption column. In some cases double interchangeable columns may be used in frames of a single procedure: at any time instance one is being used while another is being purified by the buffer solution.

Purified plasma is then reunited with the blood cells and reinfused back to the bloodstream. The entire procedure usually takes several hours, depending on the volume of blood being treated.
1. Okafor C, et al. J Clin Apheresis, 25:240-249, 2010
2. Schwartz at al. Journal of Clinical Apheresis 31:149-162 (2016)

In some of the embodiments, the treatment effect can be achieved by treating by such procedure all plasma of the patient, twice all plasma of patient at the same procedure, about ⅘ of all plasma of the patient, about ⅗ of all plasma of the patient, more than half of all plasma of the patient, more than ⅕ of all plasma of the patient.

The therapeutic effect, including but not limited to anti-aging effect of the procedure can be measured by the methods described in this application and other known in the art, e.g. but not limited to measuring in 6 months or in 1 year or in 2 years after the procedure aging biomarkers, the state of the age related declines, including but not limited to selected from the Table above titled “Declines”.

### Example 28 Animal Disease Models

The devices, agents, pharmaceutical compositions and treatment methods of this disclosure are useful for treatment and prevention of range of age related diseases, declines and conditions, including but not limited to mentioned in this disclosure. Expert in the field can easily choose the animal model to show therapeutic effect of therapy.

For example, the therapeutic effect of Target protein elimination from the bloodstream on animal model of metabolic syndrome mice with diet induced obesity can be used. 15 months old C57BL/6J mice can be divided into groups (n=10): one group receives standard laboratory chow (LabDiet J L 6% Oval, Cat #5K0Q), other groups are fed on 60% fat chow (DIO Rodent Purified Diet w/60% Energy From Fat, Cat #58Y1). Groups on high fat diet can be treated with vehicle or 10 mg/kg antibodies to Target proteins once or in other example once every 2 weeks during 6 weeks via IV route. (i.e. Mouse Follistatin-related Gene Protein/FLRG Antibody (AF1255), Mouse GDF-15 Antibody (AF6385), Human/Mouse sFRP-3 Antibody (MAB1921), R&D Systems, USA). Endotoxin level of the antibodies should be <0.10 EU per 1 pg of the antibody. The affinity of all antibodies to corresponding proteins (Kd) can be measured using fluorescent MicroScale Thermophoresis method.

After 6 weeks animal weight and fasting blood glucose, insulin, triglycerides, and leptin levels can be assessed and glucose/insulin tolerance tests can be performed.

The protective effects against the high fat diet of anti-Target antibodies can be seen from the the said tests that indicate that treated mice are more protected against high fat diet than untreated.

Other anti-aging effects can be measured by the methods known in the art, including but not limited to e.g but not limited to as shown below %:

A surface of the arena is cleaned with 10% ethanol. Distance moved, velocity, and rearing activity are measured.

To assess pain sensitivity to the foot-shock apparatus used for fear-conditioning each mouse is placed in the foot-shock chamber with a front-mounted camera to visualize various pain sensitivity parameters, which encompassed four clearly defined behaviors, ‘flinch’, ‘run’, ‘vocalization’, and ‘two-paw jump’. One-second shocks of various intensities are delivered each spaced by 30 s of recovery time. Thresholds reflected the minimum shock intensity at which each behavior can be observed using the front-mounted camera video.

### Example 29 Examples of Modified Plasma/a Pharmaceutical Composition Comprising a Blood Plasma Fraction

### Examples 29A

In one example plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in zero concentration.

In another example plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in around zero concentration.

In another example plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in concentration 0.01 ng/mol.

In another example plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in concentration 0.001 ng/mol.

In another example plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in concentration at least 10 folds less than median concentration of particular protein of healthy person of 20 years.

In another example plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in concentration at least 2 folds less than median concentration of particular protein of healthy person of 20 years.

In another example a pharmaceutical composition comprising a blood plasma fraction has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in concentration at least 30% less than the concentration of particular protein in plasma from which such a pharmaceutical composition comprising a blood plasma fraction was made of, wherein the median difference of parameters of A{1} to A{28} is defined in example 9 in such pharmaceutical composition and plasma are less than 20%.

In another example a pharmaceutical composition comprising a blood plasma fraction has two or more proteins selected from the B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in zero concentration.

In another example a pharmaceutical composition comprising a blood plasma fraction has two or more proteins selected from the B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in 0.001 ng/ml concentration or less.

In another example a pharmaceutical composition comprising a blood plasma fraction has two or more proteins selected from the B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in 0.01 pg/ml concentration or less.

In another example a pharmaceutical composition comprising a blood plasma fraction has two or more proteins selected from the B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in 0.001 pg/ml concentration or less.

In another example a pharmaceutical composition comprising a blood plasma fraction has two or more proteins selected from the B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in 0.005 ng/ml concentration or less.

It is understood that in some embodiments a pharmaceutical composition comprising a blood plasma fraction of this invention can be made of the blood person of young age, e.g. less than 40 years old for many reasons, e.g. but not limited to 1) such person will benefit from the infusion of such modified plasma back to it due to the specific health condition of such person, e.g. such person has aging related disease or it is a need to increase the stress resistance of such person etc. 2) such plasma will be infused into the aged patient or other person in need, e.g. but not limited to for anti-aging treatment and using modified plasma will have better anti-aging effect then just using young plasma. 3) such plasma is created inside the bloodstream of the person by medical intervention for anti-aging or other therapeutic or health enhancement purposes etc.

Other non-limiting examples are the plasma of aged persons, e.g. from Example 9
row 1, row 3 and row 5 for path #2178,
row 1, row 3 and row 5 for path path #16434
row 1, row 3 and row 5 for path #1220
row 1, row 3 and row 5 for path #1126
further each of plasma samples described in any one of such rows is referred as Example Row, wherein all such plasma samples described by Example Rows are further referred as Example Plasma.

For clarity row 1 for path #2178 from example 9 is

| A{1} | A{2} | A{3} | A{4} | A{5} | A{6} | A{7} | A{8} | A{9} | A{10} | A{11} | A{12} | A{13} | A{14} | A{15} |
|---|---|---|---|---|---|---|---|---|---|---|---|---|---|---|
| 20 | 5.1 | NA | 71 | 0.813 | 279 | 3.26 | 6.1 | 6 | 0.164 | 0.969 | 79.6 | 97.8 | 39 | 115 |
| | | A{16} | A{17} | A{18} | A{19} | A{20} | A{21} | A{22} | A{23} | A{24} | A{25} | A{26} | A{27} | A{28} |
| | | 392.6 | 2.475 | 7.494 | 27 | 16.48 | 80 | 137.6 | 163 | 4.25 | 41 | 0.14 | 4.45 | 92 |

Wherein each of A{1} to A{28} is defined in Example 9.

### Examples 29B

In one example one or more plasma, selected from Example Plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in zero concentration.

In another example one or more plasma, selected from Example Plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in around zero concentration.

In another example one or more plasma, selected from Example Plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in 0.001 ng/ml concentration or less.

In another example one or more plasma, selected from Example Plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in 0.005 ng/ml concentration or less.

In another example one or more plasma, selected from Example Plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in 0.01 ng/ml concentration or less.

In another example one or more plasma, selected from Example Plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in 0.001 pg/ml concentration or less.

In another example one or more plasma, selected from Example Plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in 0.01 pg/ml concentration or less.

In another example one or more plasma, selected from Example Plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in concentration at least 10 folds less than median concentration of particular protein of healthy persons of 40 years.

In another example one or more plasma, selected from Example Plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in concentration equal to median concentration of particular protein of healthy persons of 20 years.

In another example one or more plasma, selected from Example Plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in concentration at least 30% less than the concentration of particular protein in plasma from which such a pharmaceutical composition comprising a blood plasma fraction was made of, wherein the median difference of parameters of A{1} to A{28} is defined in example 9 in such pharmaceutical composition and plasma are less than 20%.

In another example one or more plasma, selected from Example Plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in concentration at least 2 folds less than the concentration of particular protein in plasma from which such a pharmaceutical composition comprising a blood plasma fraction was made of, wherein the median difference of parameters of A{1} to A{28} is defined in example 9 in such pharmaceutical composition and plasma are less than 20%.

In another example one or more plasma, selected from Example Plasma has one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in concentration at least 10 folds less than the concentration of particular protein in plasma from which such a pharmaceutical composition comprising a blood plasma fraction was made of, wherein the median difference of parameters of A{1} to A{28} is defined in example 9 in such pharmaceutical composition and plasma are less than 20%.

### Examples 29C

In another example one or more plasma, selected from Example Plasma has CCDC80 in 0.05 ng/ml concentration.

In another example one or more plasma, selected from Example Plasma has CD59 in 0.005 ng/ml concentration.

In another example one or more plasma, selected from Example Plasma has CHRDL1 in 0.1 ng/ml concentration.

In another example one or more plasma, selected from Example Plasma has COL18A1 in 0.005 ng/ml concentration.

In another example one or more plasma, selected from Example Plasma has CST3 in 0.1 ng/ml concentration.

In another example one or more plasma, selected from Example Plasma has DPT in 0.01 ng/ml concentration.

In another example one or more plasma, selected from Example Plasma has EFEMP1 in 0.1 ng/ml concentration.

In another example one or more plasma, selected from Example Plasma has FAS in 0.05 ng/ml concentration.

In another example one or more plasma, selected from Example Plasma has FSTL3 in 0.1 ng/ml concentration.

In another example one or more plasma, selected from Example Plasma has GAS1 in 0.01 ng/ml concentration.

In another example one or more plasma, selected from Example Plasma has GDF15 in 0.01 ng/ml concentration.

In another example one or more plasma, selected from Example Plasma has KLK11 in 0.1 ng/ml concentration.

In another example one or more plasma, selected from Example Plasma has MMP7 in 0.01 ng/ml concentration.

In another example one or more plasma, selected from Example Plasma has NBL1 in 0.1 ng/ml concentration.

In another example one or more plasma, selected from Example Plasma has NTN in 0.001 ng/ml concentration.

In another example one or more plasma, selected from Example Plasma has POSTN in 0.1 ng/ml concentration.

In another example one or more plasma, selected from Example Plasma has PTN in 0.1 ng/ml concentration.

In another example one or more plasma, selected from Example Plasma has RELT in 0.001 ng/ml concentration.

In another example one or more plasma, selected from Example Plasma has SFRP1 in 0.001 ng/ml concentration.

In another example one or more plasma, selected from Example Plasma has SMOC1 in 0.001 ng/ml concentration.

In another example one or more plasma, selected from Example Plasma has STC1 in 0.1 ng/ml concentration.

In another example one or more plasma, selected from Example Plasma has TNFRSF1A in 0.001 ng/ml concentration.

In another example one or more plasma, selected from Example Plasma has UNC5C in 0.1 ng/ml concentration.

In another example one or more plasma, selected from Example Plasma has sFRP-3 (FRZB) in 0.1 ng/ml concentration.

In another example one or more plasma, selected from Example Plasma has TNFRSF1B in 0.001 ng/ml concentration.

In another example one or more plasma, selected from Example Plasma has RGMB in 0.1 ng/ml concentration.

In another example one or more plasma, selected from Example Plasma has CD55 in 0.001 ng/ml concentration.

In another example one or more plasma, selected from Example Plasma has BMP4 in 0.001 ng/ml concentration.

### Examples 29D

In another example one or more plasma, selected from Example Plasma has at least two or more proteins selected from B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB in zero concentration.

In another example one or more plasma, selected from Example Plasma has at least two or more proteins selected from B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB in around zero concentration.

In another example one or more plasma, selected from Example Plasma has at least two or more proteins selected from B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB in concentration 0.001 ng/ml.

In another example one or more plasma, selected from Example Plasma has at least two or more proteins selected from B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB in concentration 0.001 pg/ml.

In another example one or more plasma, selected from Example Plasma has at least two or more proteins selected from B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB in concentration 0.01 pg/ml.

In another example one or more plasma, selected from Example Plasma has at least two or more proteins selected from B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB in concentration defined for each particular protein in Examples C above and for B2M in 1 ng/ml concentration.

### Example 30 Examples of Plasma Obtained by Degradation Agents Inside Bloodstream of the Subject

Examples of plasma that can be obtained by degradation agents inside bloodstream of the subject, degrading or inhibiting the production of one or more proteins selected from the CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB can be found for relative proteins in example 29.

Examples of plasma that can be obtained by degradation agents inside bloodstream of the subject, degrading or inhibiting the production of at least two or more proteins selected from B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4 and RGMB can be found for relative proteins in example 29.

### Example 31 Example 1 of Formulations

Exemplary Injection Formulation Containing an Agent of this Disclosure for IV Administration.

The vial contains 100 mg of active ingredient, which is any one of binding elements described in this disclosure, including but not limited to:
1. Anti-Fas/Cd95 Rabbit Monoclonal Antibody; https://www.biocompare.com/9776-Antibodies/11570077-Anti-Fas-Rabbit-Monoclonal-Antibody/?pda=9776|11570077_0_1|3681|1|FAS&dfp=true; M00055; BosterBio
2. FAS monoclonal antibody (M03); https://www.biocompare.com/9776-Antibodies/8456737-FAS-monoclonal-antibody-M03-clone-3C4/?pda=9776|8456737_0_0β681|11|FAS; MAB1301-M03; Abnova Corporation
3. DPT monoclonal antibody; https://www.biocompare.com/9776-Antibodies/567248-DPT-monoclonal-antibody-M08-clone-2A11/?pda=9776|567248_0_0β681|15DPT; H00001805-M08; Abnova Corporation
4. Human Dermatopontin Antibody; https://www.biocompare.com/9776-Antibodies/1679378-Human-Dermatopontin-Affinity-Purified-Polyclonal-Ab/?firofb=true&pda=9776|1679378_0_0|3681|24|DPT; AF4629; R&D Systems
5. Anti-CD55 (DAF) (Human) mAb; https://www.biocompare.com/9776-Antibodies/597141-CD55-2C6/?pda=9776|597141_0_1|3681|3|CD55&dfp=true; D257-3; MBL International
6. CD55 Monoclonal Antibody (Bu14); https://www.biocompare.com/9776-Antibodies/4197904-CD55-Antibody/?pda=9776|4197904_0_0|3681|13|CD55; MA5-16595; Invitrogen Antibodies
7. Human Kallikrein-11 (KLK11) Antibody; https://www.biocompare.com/9776-Antibodies/11137789-Human-Kallikrein-11-KLK11-Antibody/?pda=9776|11137789_0_0|3681|14|KLK11; 32102-05111; AssayPro
8. Monoclonal Antibody to Kallikrein 11 (KLK11); https://www.biocompare.com/9776-Antibodies/10474813-Monoclonal-Antibody-to-Kallikrein-11-KLK11/?pda=9776|10474813_0_0|3681|18|KLK11; MAA669Hu22; Cloud-Clone
9. Recombinant Anti-Human KLK11 Antibody; https://www.biocompare.com/9776-Antibodies/8717220-Rcombinant-Anti-Human-KLK11-Antibody/?pda=9776|8717220_0_0|3681|2|KLK11; MOB-2362z; Creative Biolabs
10. B2M monoclonal antibody; https://www.biocompare.com/9776-Antibodies/548319-B2M-monoclonal-antibody-clone-B2M01/?pda=9776|548319_0_0|3681|6|B2M; MAB0899; Abnova Corporation
11. SMOC1 monoclonal antibody; https://www.biocompare.com/9776-Antibodies/200764-SMOC1-monoclonal-antibody-M03-clone-8F10/?pda=9776|200764_0_0|3681|15|SMOC1; H00064093-M03; Abnova Corporation
12. Human RGM-B Antibody; https://www.biocompare.com/9776-Antibodies/364038-Human-RGMB-MAb-Clone-398528/?pda=9776|364038_0_0|3681|17|RGMB; MAB3630; R&D Systems
13. Human Pleiotrophin/PTN Antibody; https://www.biocompare.com/9776-Antibodies/4462020-Human-Pleiotrophin-PTN-MAb-Clone-851406/?pda=9776|4462020_0_0∥10|PTN; MAB252; R&DSystems
14. Pab Rb x human PTN Antibody; https://www.biocompare.com/9776-Antibodies/11053511-Pab-Rb-x-human-PTN-Antibody/?firofb=true&pda=9776|11053511_0_0∥26|PTN; 331368-50ug; United States Biological
15. Cystatin C Monoclonal Antibody; https://www.biocompare.com/9776-Antibodies/2763462-Mouse-Anti-CST3-Monoclonal-Antibody-Unconjugated-Clone-5H2/?pda=9776|2763462_0_1|3681|2|Cystatin-C&dfp=true; MA5-15841; Invitrogen Antibodies
16. Cystatin C Monoclonal Antibody; https://www.biocompare.com/9776-Antibodies/9513441-Cystatin-C-Antibody-197807/?pda=9776|9513441_0_0β681|14|Cystatin-C; MA5-23890; Invitrogen Antibodies
17. FSTL3 monoclonal antibody (M06); https://www.biocompare.com/9776-Antibodies/7342246-FSTL3-monoclonal-antibody-M06-clone-2G7/?pda=9776|7342246_0_0β681|14|FSTL3%20(FLRG); H00010272-M06; Abnova
18. Fibulin 3 (FBLN3) Monoclonal Antibody; https://www.biocompare.com/9776-Antibodies/11528090-Fibulin-3-FBLN3-Monoclonal-Antibody/?firofb=true&pda=9776|11528090_0_0|3681|18|Fibulin-3; CAU29456; Biomatik
19. Anti-Human EFEMP1 Therapeutic Antibody; https://www.biocompare.com/9776-Antibodies/12006112-Anti-Human-EFEMP1-Therapeutic-Antibody-428-3C11/?pda=9776|12006112_0_0|3681|29|Fibulin-3; TAB-005CQ; Creative Biolabs
20. GDF15 monoclonal antibody; https://www.biocompare.com/9776-Antibodies/1519366-GDF15-monoclonal-antibody-clone-23B3D2H5/?pda=9776|1519366_0_0|3681|18|GDF15; MAB8161; AbnovaCorporation
21. Monoclonal Antibody to Growth Differentiation Factor 15; https://www.biocompare.com/9776-Antibodies/10484879-Monoclonal-Antibody-to-Growth-Differentiation-Factor-15-GDF15/?pda=9776|10484879_0_0|368|29|GDF15; MAC034Hu22; Cloud-Clone
22. Human RELT/TNFRSF19L; https://www.biocompare.com/9776-Antibodies/11605574-Human-RELT-TNFRSF19L-Alexa-Fluor-174-350-conjugated-Antibody/?pda=9776|11605574_0_0|368|9|TNFRSF19L; FAB1385U-100UG; R&D Systems
23. TNFRSF19L monoclonal antibody; https://www.biocompare.com/9776-Antibodies/239820-TNFRSF19L-monoclonal-antibody-M01-clone-3F8/?pda=9776|239820_0_0|3681|15|TNFRSF19L; H00084957-M01; Abnova Corporation
24. CD59 Monoclonal Antibody; https://www.biocompare.com/9776-Antibodies/472474-Mouse-AntiCD59-Monoclonal-Antibody-Unconjugated-Clone-1F5/?pda=9776|472474_0_1∥1|CD59&dfp=true; MA1-70058; InvitrogenAntibodies
25. COL18A1 monoclonal antibody; https://www.biocompare.com/9776-Antibodies/1395970-COL18A1-monoclonal-antibody-clone-KT3/?pda=9776|1395970_0_0|3681|16|COL18A1; MAB1664; Abnova Corporation
26. UNC5C monoclonal antibody; https://www.biocompare.com/9776-Antibodies/1394317-UNC5C-monoclonal-antibody-M01-clone-3C9/?pda=9776|1394317_0_0|3681|22|UNC5C; H00008633-M01; Abnova Corporation
27. Periostin monoclonal antibody; https://www.biocompare.com/9776-Antibodies/1377325-Periostin-mAb-47/?pda=9776|1377325_0_0|3681|3|POSTN; ADI-905-889-100; Enzo Life Sciences
28. Anti-SFRP1 Rabbit Monoclonal Antibody; https://www.biocompare.com/9776-Antibodies/11563502-Anti-SFRP1-Rabbit-Monoclonal-Antibody/?pda=9776|11563502_0_1|3681|1|SFRP1&dfp=true; M01968; BosterBio
29. Anti-ATR (Human) mAb; https://www.biocompare.com/9776-Antibodies/352308-ATR-Ataxia-Telangiecasia-and-Rad3-Related-Monoclonal-Antibody/?firofb=true&pda=9776|352308_0_0|3681|30|SFRP1; M123-3; MBL International
30. Rabbit Monoclonal antibody against Stanniocalcin-1 (STC1); https://www.biocompare.com/9776-Antibodies/4251437-Rabbit-Monoclonal-antibody-against-Stanniocalcin-1-STC1/?pda=9776|4251437_0_0|3681|13|STC1; TA311078; OriGeneTechnologies
31. Anti-SFRP-3/FRZB Antibody; https://www.biocompare.com/9776-Antibodies/336046-AntiFRZB-Monoclonal-Antibody/?pda=9776|336046_0_0|3681|4|sFRP-3; LS-C10135-100; LifeSpan BioSciences
32. BMP4 monoclonal antibody; https://www.biocompare.com/9776-Antibodies/1369552-BMP4-mAb-26H16/?firofb=true&pda=9776|1369552_0_0|3681|10|BMP4; ADI-905-757-100; Enzo Life Sciences
33. Anti-BMP4 Antibody; https://www.biocompare.com/9776-Antibodies/1378061-AntiBMP4-Monoclonal-Antibody/?pda=9776|1378061_0_1|3681|2|BMP4&dfp=true; LS-C104439-500; LifeSpan BioSciences
34. Anti-TNFRSF1B/TNFR2 Antibody (Biotin); https://www.biocompare.com/9776-Antibodies/358286-AntiTNFRSF1B-TNFR2-Monoclonal-Antibody/?pda=9776|358286_0_1|3681|1|TNFRSF1B&dfp=true; LS-C18375-50; LifeSpan BioSciences
35. Monoclonal Antibody to Tumor Necrosis Factor Receptor Superfamily, Member 1B (TNFRSF1B); https://www.biocompare.com/9776-Antibodies/10484386-Monoclonal-Antibody-to-Tumor-Necrosis-Factor-Receptor-Superfamily-Member-1B-TNFRSF1B/?pda=9776|10484386_0_0|3681|20|TNFRSF1B; MAB504Hu21; Cloud-Clone
36. TNFR2 (soluble) Monoclonal Antibody; https://www.biocompare.com/9776-Antibodies/7009989-TNF-Receptor-II-CD120b-Monoclonal-Antibody-897C2G9/?pda=9776|7009989_0_0|3681|26|TNFRSF1B; AHR3022; Invitrogen Antibodies
37. TNFRSF1A monoclonal antibody; https://www.biocompare.com/9776-Antibodies/10651951-TNFRSF1A-monoclonal-antibody-clone-H398/?pda=9776|10651951_0_0|3681|9|TNFRSF1A; MAB13968; AbnovaCorporation
38. TNFRSF1A mouse monoclonal antibody; https://www.biocompare.com/9776-Antibodies/3519589-Luminex-validated-purified-TNF-R-Mouse-monoclonal-antibody-clone-TBP-1-capture/?pda=9776|3519589_0_0|3681|13|TNFRSF1A; TA600021; OriGene Technologies
39. NTN1 monoclonal antibody; https://www.biocompare.com/9776-Antibodies/2331911-NTN1-monoclonal-antibody-M01-clone-5H8/?pda=9776|2331911_0_0∥8|NTN1; H00009423-M01; AbnovaCorporation
40. Netrin-1 monoclonal antibody; https://www.biocompare.com/9776-Antibodies/2797802-Netrin-1-mAb-Nora-1/?pda=9776|2797802_0_0∥24|NTN1; ALX-804-838-Cl00; Enzo Life Sciences
41. Anti-CCDC80; https://www.biocompare.com/9776-Antibodies/11562764-Anti-CCDC80-Antibody/?pda=9776|11562764_0_1∥1|CCDC80&dfp=true; A08605; BosterBio
42. Anti-CCDC80 Antibody; https://www.biocompare.com/9776-Antibodies/5765938-Anti-CCDC80-Antibody-aa635-906/?pda=9776|5765938_0_1∥2|CCDC80&dfp=true; LS-C300023-100; LifeSpan BioSciences
43. NBL1 Antibody; https://www.biocompare.com/9776-Antibodies/4684394-NBL1-Antibody-OAAF02262/?pda=9776|4684394_0_1∥1|NBL1&dfp=true; OAAF02262; Aviva Systems Biology
44. DAN/NBL1 Antibody; https://www.biocompare.com/9776-Antibodies/8568292-DAN-NBL1-Antibody-38G8/?pda=9776|8568292_0_1∥3|NBL1&dfp=true; NBP2-42637; Novus Biologicals
45. Fibulin 3 (FBLN3) Monoclonal Antibody; https://www.biocompare.com/9776-Antibodies/11528090-Fibulin-3-FBLN3-Monoclonal-Antibody/?firofb=true&pda=9776|11528090_0_0|3681|18|Fibulin-3; CAU29456; Biomatik
46. Anti-Human EFEMP1 Therapeutic Antibody; https://www.biocompare.com/9776-Antibodies/12006112-Anti-Human-EFEMP1-Therapeutic-Antibody-428-3C11/?pda=9776|12006112_0_0|3681|29|Fibulin-3; TAB-005CQ; CreativeBiolabs
47. MMP-7 monoclonal antibody (2D3); https://www.biocompare.com/9776-Antibodies/4630707-MMP-7-mAb-2D3/?pda=9776|4630707_0_0|3681|12|MMP7,%20Matrilysin,%20P09237; ADI-905-1013-0100; Enzo Life Sciences
48. Anti-MMP7; https://www.biocompare.com/9776-Antibodies/356046-AntiMMP7-MonoclonalAntibody/?pda=9776|356046_0_1|3681|2|MMP7,%20Matrilysin,%20P09237 &dfp=true; LS-C8765-100; LifeSpan BioSciences
49. Ingredient selected from the group: anti-CCDC80 antibody, anti-CCDC80 human antibody, anti-CD59 antibody, anti-CD59 human antibody, anti-CHRDL1 antibody, anti-CHRDL1 human antibody, anti-COL18A1 antibody, anti-COL18A1 human antibody, anti-CST3 antibody, anti-CST3 human antibody, anti-DPT antibody, anti-DPT human antibody, anti-EFEMP1 antibody, anti-EFEMP1 human antibody, anti-FAS antibody, anti-FAS human antibody, anti-FSTL3 antibody, anti-FSTL3 human antibody, anti-GAS1 antibody, anti-GAS1 human antibody, anti-GDF15 antibody, anti-GDF15 human antibody, anti-KLK11 antibody, anti-KLK11 human antibody, anti-MMP7 antibody, anti-MMP7 human antibody, anti-NBL1 antibody, anti-NBL1 human antibody, anti-NTN1 antibody, anti-NTN1 human antibody, anti-POSTN antibody, anti-POSTN human antibody, anti-PTN antibody, anti-PTN human antibody, anti-RELT antibody, anti-RELT human antibody, anti-SFRP1 antibody, anti-SFRP1 human antibody, anti-SMOC1 antibody, anti-SMOC1 human antibody, anti-STC1 antibody, anti-STC1 human antibody, anti-TNFRSF1A antibody, anti-TNFRSF1A human antibody, anti-UNC5C antibody, anti-UNC5C human antibody, anti-sFRP-3 antibody, anti-sFRP-3 human antibody, anti-TNFRSF1B antibody, anti-TNFRSF1B human antibody, anti-CD55 antibody, anti-CD55 human antibody, anti-BMP4 antibody, anti-BMP4 human antibody, anti-RGMB antibody, anti-RGMB human antibody, anti-GDF15 human antibody, an anti-FSTL3 human antibody, an anti-BMP4 human antibody or an anti-FRZB (sFRP3) human antibody, anti-CCDC80 monoclonal humanized antibody, anti-CD59 monoclonal humanized antibody, anti-CHRDL1 monoclonal humanized antibody, anti-COL18A1 monoclonal humanized antibody, anti-CST3 monoclonal humanized antibody, anti-DPT monoclonal humanized antibody, anti-EFEMP1 monoclonal humanized antibody, anti-FAS monoclonal humanized antibody, anti-FSTL3 monoclonal humanized antibody, anti-GAS1 monoclonal humanized antibody, anti-GDF15 monoclonal humanized antibody, anti-KLK11 monoclonal humanized antibody, anti-MMP7 monoclonal humanized antibody, anti-NBL1 monoclonal humanized antibody, anti-NTN1 monoclonal humanized antibody, anti-POSTN monoclonal humanized antibody, anti-PTN monoclonal humanized antibody, anti-RELT monoclonal humanized antibody, anti-SFRP1 monoclonal humanized antibody, anti-SMOC1 monoclonal humanized antibody, anti-STC1 monoclonal humanized antibody, anti-TNFRSF1A monoclonal humanized antibody, anti-UNC5C monoclonal humanized antibody, anti-sFRP-3 monoclonal humanized antibody, anti-TNFRSF1B monoclonal humanized antibody, anti-CD55 monoclonal humanized antibody, anti-BMP4 monoclonal humanized antibody, anti-RGMB monoclonal humanized antibody, anti-GDF15 monoclonal humanized antibody, an anti-FSTL3 monoclonal humanized antibody, an anti-BMP4 monoclonal humanized antibody, or an anti-FRZB (sFRP3) monoclonal humanized antibody.
50. Somamers described in this application SL000283, SL010390, SL004557, SL009400, SL000403, SL001777, SL008178, SL006527, SL002731, SL009324, SL000658, SL003869, SL002763, SL000525, SL005156, SL012395, SL005084, SL002704, SL005213, SL003770, SL011888, SL005789, SL001992, SL005230, SL004650.
51. Any of the antibodies selected from Additional examples of GDF15 binding elements from Example 1, etc. as a powder for injection. Powder for Injection is to be reconstituted with sterile water for injections and further diluted in 0,9% sodium chloride solution for infusion, After reconstitution, each vial contains substance for injection 100 mg/10 mL, Inactive Ingredients: sodium phosphate monobasic monohydrate, sodium phosphate dibasic dihydrate, sucrose and polysorbate 80.

In preferred embodiment, the agents for administration—antibodies and other binding elements described in this application or alike molecules should be requested for delivery or produced in the form suitable for injection or other administration into animal or human subject, e.g. free of endotoxins, azides and other elements or objects not suitable for animal and human administration. In case e.g. antibodies are delivered with such elements or objects the binding element should be purified from such objects by the method known in the art, e.g. described in GE Healthcare Antibody Purification Handbook 18-1037-46 AD or by rules applicable for medications production or for medication used in clinical trials. In case if administered in humans in preferred embodiment antibodies should be humanized or human or in any other way optimized to minimize potential side effects.

### Example 31

Exemplary IV Injection Formulation Containing an Agent of this Disclosure.

The 100 mg of any one active ingredients of Example 30 or any two of active ingredients of Example 30 targeting different Targets are formulated in 240 mg α,α-trehalose dihydrate, 23.2 mg sodium phosphate (monobasic, monohydrate), 4.8 mg sodium phosphate (dibasic, anhydrous), 1.6 mg polysorbate 20, and Water for Injection, USP.

### Example 32 Example of Pharmaceutical Composition, Comprising Blood Plasma, Wherein One or More Targets are Bound by Binding Element or Inhibited

In some embodiments, pharmaceutical composition, comprising blood plasma can be generated inside blood stream of the subject by administering binding elements or inhibitors of Targets into the subject that will have the effect comparable with the physical reduction of these proteins by the device of this invention.

The non-limiting examples of such pharmaceutical compositions, comprising blood plasma can be any one of one or more plasma, selected from Example Plasma from Example 29, wherein one or more Targets are bound by any one of binding elements described in this application related to particular Target, including but not limited to any one selected from Active ingredients from Example 30.

What is claimed is:

1. A protein binding device, wherein the protein binding device comprises a housing, wherein the housing defines a lumen and the housing further comprises an inlet port for receiving fluid to the lumen, and an outlet port for releasing fluid from the lumen, wherein the lumen comprises binding elements, wherein the binding elements selectively bind a protein selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

2. A pharmaceutical composition provided as part of an anti-aging treatment or for treating or preventing an age-related disease or disorder comprising an agent configured to bind to, inhibit, or degrade a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB; and at least one pharmaceutically acceptable excipient.

3. The pharmaceutical composition of claim 2, wherein the protein is FSTL3, wherein the pharmaceutical composition is devoid of or excludes compounds or molecules for treating insulin resistance, diastolic heart failure, and obesity.

4. The pharmaceutical composition of claim 2, wherein the agent is selected from the group consisting of: a protein, a polymer, an aptamer, a SOMAmer, a peptide, a virus, a small molecule, a nanoparticle, an antibody, a monoclonal antibody, a polyclonal antibody, a humanized monoclonal antibody, a human monoclonal antibody, a human or humanized polyclonal antibody, an anti-GDF15 human antibody, an anti-FSTL3 human antibody, an anti-BMP4 human antibody, and an anti-FRZB (sFRP3) human antibody.

5. The pharmaceutical composition of claim 4, wherein the agent is bound to a particle.

6. A pharmaceutical composition comprising a blood plasma fraction, wherein the blood plasma fraction comprises a negligible amount of a protein selected from the group consisting of CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB.

7. The pharmaceutical composition of claim 6, wherein the negligible amount is between about 0.008 pg/mL and about 15,000 ng/mL.

8. The pharmaceutical composition of claim 6, comprising one or more biomarkers characteristic of aged blood.

9. The pharmaceutical composition of claim 8, wherein one of the biomarkers characteristic of aged blood is a biological age of the subject from whose blood the pharmaceutical composition is produced, wherein the biological age of the subject is greater than 45 years of age.

10. A method of providing an anti-aging treatment or of treating or preventing an age-related disease or disorder of a subject comprising reducing, inhibiting, or degrading a protein selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in the blood of the subject.

11. The method of claim 10, further comprising administering to the subject a gene therapy.

12. The method of claim 10, wherein the age-related disease or disorder is associated with an alleviated level of a protein selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB in a bodily fluid of the subject.

13. The method of claim 10, wherein the age-related disease or disorder is selected from the group consisting of frailty, Alzheimer's disease, Parkinson's disease, Huntington's diseases, cardiovascular disease, renal failure, muscle wasting or cachexia, osteopenia or osteoporosis, obesity, insulin resistance or diabetes, diverse adult-onset cancers, atherosclerosis, cardiovascular disease, adult cancer, arthritis, cataracts, osteoporosis, type 2 diabetes, hypertension, age-progressive dementia; amyotrophic lateral sclerosis, stroke, atrophic gastritis, osteoarthritis, NASH, camptocormia, chronic obstructive pulmonary disease, coronary artery disease, dopamine dysregulation syndrome, metabolic syndrome, effort incontinence, Hashimoto's thyroiditis, heart failure, late life depression, immunosenescence, age related decline in immune response to vaccines, age related decline in response to immunotherapy, myocardial infarction, acute coronary syndrome, sarcopenia, sarcopenic obesity, senile osteoporosis, urinary incontinence, stroke, atrophic gastritis, camptocormia, chronic obstructive pulmonary disease, coronary artery disease, dopamine dysregulation syndrome, late life depression, osteoarthritis, chronic fatigue syndrome, senile dementia, mild cognitive impairment due to aging, Creutzfeldt-Jakob disease, stroke, CNS cerebral senility, pre-diabetes, diabetes, peripheral arterial disease, aortic valve disease, stroke, Lewy body disease, progressive subcortical gliosis, progressive supranuclear palsy, thalamic degeneration syndrome, hereditary aphasia, myoclonus epilepsy, macular degeneration, pressure ulcers, delirium, progressive subcortical gliosis, progressive supranuclear palsy, thalamic degeneration syndrome, hereditary aphasia, myoclonus epilepsy, and metabolic disorder.

14. The method of claim 10, wherein the anti-aging treatment is selected from the group consisting of a treatment leading to prevention, amelioration or lessening the effects of aging; decreasing or delaying an increase in a biological age of the subject; slowing a rate of aging of the subject; prevention, amelioration or lessening the effects of frailty; prevention, amelioration or lessening the effects of at least one of an aging-related disease or conditions; increasing a health span or lifespan of the subject; increasing a stress resistance or resilience of the subject; increasing a rate or other enhancement of recovery after surgery, radiotherapy, disease and/or any other stress; prevention, amelioration or lesion the effects of menopausal syndrome; restoring reproductive function; elimination or lessening the spread of senescent cells; modulation of at least one biomarker of aging; a decrease in a rate of wrinkle development; and decrease in a rate of hair greying.

15. The method of claim 10, wherein the anti-aging treatment is selected from the group consisting of a treatment related to changing a blood parameter, a heart rate, a cognitive function, a bone density, a basal metabolic rate, a systolic blood pressure, a heel bone mineral density (BMD), a heel quantitative ultrasound index (QUI), a heel broadband ultrasound attenuation, a forced expiratory volume in 1-second (FEV1), forced vital capacity (FVC), a peak expiratory flow (PEF), a duration to first press of snap-button in each round, a reaction time, a mean time to correctly identify matches, a right or left hand grip strength, a whole body fat-free mass, a leg fat-free mass, a time for recovery after a stress-inducing event, a resistance to radiation, a morbidity risk, and a mortality risk of the subject.

16. A method of providing an anti-aging treatment or of treating or preventing an age-related disease or disorder of a subject, comprising administering to the subject a pharmaceutical composition comprising an inhibitor of a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB; and at least one pharmaceutically acceptable excipient.

17. A method of providing an anti-aging treatment or of treating or preventing an age-related disease or disorder of a subject, comprising administering to the subject a pharmaceutical composition comprising a blood plasma fraction, wherein the blood plasma fraction comprises a negligible amount of a protein selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein the negligible amount is between about 0.008 pg/mL and about 15,000 ng/mL.

18. A kit, comprising:
a pharmaceutical composition comprising:
an agent configured to bind to, inhibit, or degrade a protein selected from the group consisting of: B2M, CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, wherein the agent is selected from the group consisting of: a protein, a polymer, an aptamer, a SOMAmer, a peptide, a virus, a small molecule, a nanoparticle, an antibody, a monoclonal antibody, a polyclonal antibody, a humanized monoclonal antibody, a human monoclonal antibody, a human or humanized polyclonal antibody, an anti-GDF15 human antibody, an anti-FSTL3 human antibody, an anti-BMP4 human antibody, and an anti-FRZB (sFRP3) human antibody, and
at least one pharmaceutically acceptable excipient; and
an instruction for using the pharmaceutical composition as part of the anti-aging treatment or to treat or prevent an age-related disease or disorder.

19. A tangible medium comprising a computer program, which, when executed by one or more processors of a computing device, causes the computing device to attribute information regarding a protein binding device, another device, or a therapeutic agent with information about reducing, binding, inhibiting, or degrading at least one protein within the blood of the subject, wherein the protein is selected from the group consisting of: CCDC80, CD59, CHRDL1, COL18A1, CST3, DPT, EFEMP1, FAS, FSTL3, GAS1, GDF15, KLK11, MMP7, NBL1, NTN1, POSTN, PTN, RELT, SFRP1, SMOC1, STC1, TNFRSF1A, UNC5C, sFRP-3, TNFRSF1B, CD55, BMP4, and RGMB, and wherein the information about reducing, binding, inhibiting or degrading the protein is associated with an anti-aging treatment.

20. The tangible medium of claim 19, wherein the tangible medium is a computer storage medium.


SEQUENCE LISTING (39 sequences)

SEQ ID NO: 1 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 2 - protein, 345 aa, Homo sapiens
MVAALLGGGGEARGGTVPGAWLCLMALLQLLGSAPRGSGLAHGRRLICWQALLQCQGEPECSYAYNQYAEACAPVLAQHGGGDAPGAAAAAFPASAASFSSRWRCPSHCISALIQLNHTRRGPALEDCDCAQDENCKSTKRAIEPCLPRTSGGGAGGPGAGGVMGCTEARRRCDRDSRCNLALSRYLTYCGKVFNGLRCTDECRTVIEDMLAMPKAALLNDCVCDGLERPICESVKENMARLCFGAELGNGPGSSGSDGGLDDYYDEDYDDEQRTGGAGGEQPLDDDDGVPHPPRPGSGAAASGGRGDLPYGPGRRSSGGGGRLAPRGAWTPLASILLLLLGPLF

SEQ ID NO: 3 - protein, 434 aa, Homo sapiens
MLPARCARLLTPHLLLVLVQLSPARGHRTTGPRFLISDRDPQCNLHCSRTQPKPICASDGRSYESMCEYQRAKCRDPTLGVVHRGRCKDAGQSKCRLERAQALEQAKKPQEAVFVPECGEDGSFTQVQCHTYTGYCWCVTPDGKPISGSSVQNKTPVCSGSVTDKPLSQGNSGRKDDGSKPTPTMETQPVFDGDEITAPTLWIKHLVIKDSKLNNTNIRNSEKVYSCDQERQSALEEAQQNPREGIVIPECAPGGLYKPVQCHQSTGYCWCVLVDTGRPLPGTSTRYVMPSCESDARAKTTEADDPFKDRELPGCPEGKKMEFITSLLDALTTDMVQAINSAAPTGGGRFSEPDPSHTLEERVVHWYFSQLDSNSSNDINKREMKPFKRYVKKKAKPKKCARRFTDYCDLNKDKVISLPELKGCLGVSKEGRLV

SEQ ID NO: 4 - protein, 267 aa, Homo sapiens
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 5 - protein, 931 aa, Homo sapiens
MRKGLRATAARCGLGLGYLLQMLVLPALALLSASGTGSAAQDDDFFHELPETFPSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTPWSKWSTCGTECTHWRRRECTAPAPKNGGKDCDGLVLQSKNCTDGLCMQTAPDSDDVALYVGIVIAVIVCLAISVVVALFVYRKNHRDFESDIIDSSALNGGFQPVNIKAARQDLLAVPPDLTSAAAMYRGPVYALHDVSDKIPMTNSPILDPLPNLKIKVYNTSGAVTPQDDLSEFTSKLSPQMTQSLLENEALSLKNQSLARQTDPSCTAFGSFNSLGGHLIVPNSGVSLLIPAGAIPQGRVYEMYVTVHRKETMRPPMDDSQTLLTPVVSCGPPGALLTRPVVLTMHHCADPNTEDWKILLKNQAAQGQWEDVVVVGEENFTTPCYIQLDAEACHILTENLSTYALVGHSTTKAAAKRLKLAIFGPLCCSSLEYSIRVYCLDDTQDALKEILHLERQMGGQLLEEPKALHFKGSTHNLRLSIHDIAHSLWKSKLLAKYQEIPFYHVWSGSQRNLHCTFTLERFSLNTVELVCKLCVRQVEGEGQIFQLNCTVSEEPTGIDLPLLDPANTITTVTGPSAFSIPLPIRQKLCSSLDAPQTRGHDWRMLAHKLNLDRYLNYFATKSSPTGVILDLWEAQNFPDGNLSMLAAVLEEMGRHETVVSLAAEGQY

SEQ ID NO: 6 - protein, 201 aa, Homo sapiens
MDLSLLWVLLPLVTMAWGQYGDYGYPYQQYHDYSDDGWVNLNRQGFSYQCPQGQVIVAVRSIFSKKEGSDRQWNYACMPTPQSLGEPTECWWEEINRAGMEWYQTCSNNGLVAGFQSRYFESVLDREWQFYCCRYSKRCPYSCWLTTEYPGHYGEEMDMISYNYDYYIRGATTTFSAVERDRQWKFIMCRMTEYDCEFANV

SEQ ID NO: 7 - protein, 493 aa, Homo sapiens
MLKALFLTMLTLALVKSQDTEETITYTQCTDGYEWDPVRQQCKDIDECDIVPDACKGGMKCVNHYGGYLCLPKTAQIIVNNEQPQQETQPAEGTSGATTGVVAASSMATSGVLPGGGFVASAAAVAGPEMQTGRNNFVIRRNPADPQRIPSNPSHRIQCAAGYEQSEHNVCQDIDECTAGTHNCRADQVCINLRGSFACQCPPGYQKRGEQCVDIDECTIPPYCHQRCVNTPGSFYCQCSPGFQLAANNYTCVDINECDASNQCAQQCYNILGSFICQCNQGYELSSDRLNCEDIDECRTSSYLCQYQCVNEPGKFSCMCPQGYQVVRSRTCQDINECETTNECREDEMCWNYHGGFRCYPRNPCQDPYILTPENRCVCPVSNAMCRELPQSIVYKYMSIRSDRSVPSDIFQIQATTIYANTINTFRIKSGNENGEFYLRQTSPVSAMLVLVKSLSGPREHIVDLEMLTVSSIGTFRTSSVLRLTIIVGPFSF

SEQ ID NO: 8 - protein, 247 aa, Homo sapiens
MLQNSAVLLVLVISASATHEAEQNDSVSPRKSRVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESA

SEQ ID NO: 9 - protein, 263 aa, Homo sapiens
MRPGAPGPLWPLPWGALAWAVGFVSSMGSGNPAPGGVCWLQQGQEATCSLVLQTDVTRAECCASGNIDTAWSNLTHPGNKINLLGFLGLVHCLPCKDSCDGVECGPGKACRMLGGRPRCECAPDCSGLPARLQVCGSDGATYRDECELRAARCRGHPDLSVMYRGRCRKSCEHVVCPRPQSCVVDQTGSAHCVVCRAAPCPVPSSPGQELCGNNNVTYISSCHMRQATCFLGRSIGVRHAGSCAGTPEEPPGGESAEEEENFV

SEQ ID NO: 10 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 11 - protein, 604 aa, Homo sapiens
MMRAVWEALAALAAVACLVGAVRGGPGLSMFAGQAAQPDPCSDENGHPRRCIPDFVNAAFGKDVRVSSTCGRPPARYCVVSERGEERLRSCHLCNASDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYFYAVSDLQVGGRCKCNGHAARCVRDRDDSLVCDCRHNTAGPECDRCKPFHYDRPWQRATAREANECVACNCNLHARRCRFNMELYKLSGRKSGGVCLNCRHNTAGRHCHYCKEGYYRDMGKPITHRKACKACDCHPVGAAGKTCNQTTGQCPCKDGVTGITCNRCAKGYQQSRSPIAPCIKIPVAPPTTAASSVEEPEDCDSYCKASKGKLKINMKKYCKKDYAVQIHILKADKAGDWWKFTVNIISVYKQGTSRIRRGDQSLWIRSRDIACKCPKIKPLKKYLLLGNAEDSPDQSGIVADKSSLVIQWRDTWARRLRKFQQREKKGKCKKA

SEQ ID NO: 12 - protein, 437 aa, Homo sapiens
MGLRAAPSSAAAAAAEVEQRRSPGLCPPPLELLLLLLFSLGLLHAGDCQQPAQCRIQKCTTDFVSLTSHLNSAVDGFDSEFCKALRAYAGCTQRTSKACRGNLVYHSAVLGISDLMSQRNCSKDGPTSSTNPEVTHDPCNYHSHAGAREHRRGDQNPPSYLFCGLFGDPHLRTFKDNFQTCKVEGAWPLIDNNYLSVQVTNVPVVPGSSATATNKITIIFKAHHECTDQKVYQAVTDDLPAAFVDGTTSGGDSDAKSLRIVERESGHYVEMHARYIGTTVFVRQVGRYLTLAIRMPEDLAMSYEESQDLQLCVNGCPLSERIDDGQGQVSAILGHSLPRTSLVQAWPGYTLETANTQCHEKMPVKDIYFQSCVFDLLTTGDANFTAAAHSALEDVEALHPRKERWHIFPSSGNGTPRGGSDLSVSLGLTCLILIVFL

SEQ ID NO: 13 - protein, 1754 aa, Homo sapiens
MAPYPCGCHILLLLFCCLAAARANLLNLNWLWFNNEDTSHAATTIPEPQGPLPVQPTADTTTHVTPRNGSTEPATAPGSPEPPSELLEDGQDTPTSAESPDAPEENIAGVGAEILNVAKGIRSFVQLWNDTVPTESLARAETLVLETPVGPLALAGPSSTPQENGTTLWPSRGIPSSPGAHTTEAGTLPAPTPSPPSLGRPWAPLTGPSVPPPSSGRASLSSLLGGAPPWGSLQDPDSQGLSPAAAAPSQQLQRPDVRLRTPLLHPLVMGSLGKHAAPSAFSSGLPGALSQVAVTTLTRDSGAWVSHVANSVGPGLANNSALLGADPEAPAGRCLPLPPSLPVCGHLGISRFWLPNHLHHESGEQVRAGARAWGGLLQTHCHPFLAWFFCLLLVPPCGSVPPPAPPPCCQFCEALQDACWSRLGGGRLPVACASLPTQEDGYCVLIGPAAERISEEVGLLQLLGDPPPQQVTQTDDPDVGLAYVFGPDANSGQVARYHFPSLFFRDFSLLFHIRPATEGPGVLFAITDSAQAMVLLGVKLSGVQDGHQDISLLYTEPGAGQTHTAASFRLPAFVGQWTHLALSVAGGFVALYVDCEEFQRMPLARSSRGLELEPGAGLFVAQAGGADPDKFQGVIAELKVRRDPQVSPMHCLDEEGDDSDGASGDSGSGLGDARELLREETGAALKPRLPAPPPVTTPPLAGGSSTEDSRSEEVEEQTTVASLGAQTLPGSDSVSTWDGSVRTPGGRVKEGGLKGQKGEPGVPGPPGRAGPPGSPCLPGPPGLPCPVSPLGPAGPALQTVPGPQGPPGPPGRDGTPGRDGEPGDPGEDGKPGDTGPQGFPGTPGDVGPKGDKGDPGVGERGPPGPQGPPGPPGPSFRHDKLTFIDMEGSGFGGDLEALRGPRGFPGPPGPPGVPGLPGEPGRFGVNSSDVPGPAGLPGVPGREGPPGFPGLPGPPGPPGREGPPGRTGQKGSLGEAGAPGHKGSKGAPGPAGARGESGLAGAPGPAGPPGPPGPPGPPGPGLPAGFDDMEGSGGPFWSTARSADGPQGPPGLPGLKGDPGVPGLPGAKGEVGADGVPGFPGLPGREGIAGPQGPKGDRGSRGEKGDPGKDGVGQPGLPGPPGPPGPVVYVSEQDGSVLSVPGPEGRPGFAGFPGPAGPKGNLGSKGERGSPGPKGEKGEPGSIFSPDGGALGPAQKGAKGEPGFRGPPGPYGRPGYKGEIGFPGRPGRPGMNGLKGEKGEPGDASLGFGMRGMPGPPGPPGPPGPPGTPVYDSNVFAESSRPGPPGLPGNQGPPGPKGAKGEVGPPGPPGQFPFDFLQLEAEMKGEKGDRGDAGQKGERGEPGGGGFFGSSLPGPPGPPGPPGPRGYPGIPGPKGESIRGQPGPPGPQGPPGIGYEGRQGPPGPPGPPGPPSFPGPHRQTISVPGPPGPPGPPGPPGTMGASSGVRLWATRQAMLGQVHEVPEGWLIFVAEQEELYVRVQNGFRKVQLEARTPLPRGTDNEVAALQPPVVQLHDSNPYPRREHPHPTARPWRADDILASPPRLPEPQPYPGAPHHSSYVHLRPARPTSPPAHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 14 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 15 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 16 - protein, 325 aa, Homo sapiens
MVCGSPGGMLLLRAGLLALAALCLLRVPGARAAACEPVRIPLCKSLPWNMTKMPNHLHHSTQANAILAIEQFEGLLGTHCSPDLLFFLCAMYAPICTIDFQHEPIKPCKSVCERARQGCEPILIKYRHSWPENLACEELPVYDRGVCISPEAIVTADGADFPMDSSNGNCRGASSERCKCKPIRATQKTYFRNNYNYVIRAKVKEIKTKCHDVTAVVEVKEILKSSLVNIPRDTVNLYTSSGCLCPPLNVNEEYIIMGYEDEERSRLLLVEGSIAEKWKDRLGKKVKRWDMKLRHLGLSKSDSSNSDSTQSQKSGRNSNPRQARN

SEQ ID NO: 17 - protein, 950 aa, Homo sapiens
MTWRMGPRFTMLLAMWLVCGSEPHPHATIRGSHGGRKVPLVSPDSSRPARFLRHTGRSRGIERSTLEEPNLQPLQRRRSVPVLRLARPTEPPARSDINGAAVRPEQRPAARGSPREMIRDEGSSARSRMLRFPSGSSSPNILASFAGKNRVWVISAPHASEGYYRLMMSLLKDDVYCELAERHIQQIVLFHQAGEEGGKVRRITSEGQILEQPLDPSLIPKLMSFLKLEKGKFGMVLLKKTLQVEERYPYPVRLEAMYEVIDQGPIRRIEKIRQKGFVQKCKASGVEGQVVAEGNDGGGGAGRPSLGSEKKKEDPRRAQVPPTRESRVKVLRKLAATAPALPQPPSTPRATTLPPAPATTVTRSTSRAVTVAARPMTTTAFPTTQRPWTPSPSHRPPTTTEVITARRPSVSENLYPPSRKDQHRERPQTTRRPSKATSLESFTNAPPTTISEPSTRAAGPGRFRDNRMDRREHGHRDPNVVPGPPKPAKEKPPKKKAQDKILSNEYEEKYDLSRPTASQLEDELQVGNVPLKKAKESKKHEKLEKPEKEKKKKMKNENADKLLKSEKQMKKSEKKSKQEKEKSKKKKGGKTEQDGYQKPTNKHFTQSPKKSVADLLGSFEGKRRLLLITAPKAENNMYVQQRDEYLESFCKMATRKISVITIFGPVNNSTMKIDHFQLDNEKPMRVVDDEDLVDQRLISELRKEYGMTYNDFFMVLTDVDLRVKQYYEVPITMKSVFDLIDTFQSRIKDMEKQKKEGIVCKEDKKQSLENFLSRFRWRRRLLVISAPNDEDWAYSQQLSALSGQACNFGLRHITILKLLGVGEEVGGVLELFPINGSSVVEREDVPAHLVKDIRNYFQVSPEYFSMLLVGKDGNVKSWYPSPMWSMVIVYDLIDSMQLRRQEMAIQQSLGMRCPEDEYAGYGYHSYHQGYQDGYQDDYRHHESYHHGYPY

SEQ ID NO: 18 - protein, 450 aa, Homo sapiens
MGGMKYIFSLLFFLLLEGGKTEQVKHSETYCMFQDKKYRVGERWHPYLEPYGLVYCVNCICSENGNVLCSRVRCPNVHCLSPVHIPHLCCPRCPDSLPPVNNKVTSKSCEYNGTTYQHGELFVAEGLFQNRQPNQCTQCSCSEGNVYCGLKTCPKLTCAFPVSVPDSCCRVCRGDGELSWEHSDGDIFRQPANREARHSYHRSHYDPPPSRQAGGLSRFPGARSHRGALMDSQQASGTIVQIVINNKHKHGQVCVSNGKTYSHGESWHPNLRAFGIVECVLCTCNVTKQECKKIHCPNRYPCKYPQKIDGKCCKVCPGKKAKELPGQSFDNKGYFCGEETMPVYESVFMEDGETTRKIALETERPPQVEVHVWTIRKGILQHFHIEKISKRMFEELPHFKLVTRTTLSQWKIFTEGEAQISQMCSSRVCRTELEDLVKVLYLERSEKGHC

SEQ ID NO: 19 - protein, 181 aa, Homo sapiens
MMLRVLVGAVLPAMLLAAPPPINKLALFPDKSAWCEAKNITQIVGHSGCEAKSIQNRACLGQCFSYSVPNTFPQSTESLVHCDSCMPAQSMWEIVTLECPGHEEVPRVDKLVEKILHCSCQACGKEPSHEGLSVYVQGEDGPGSQPGTHPHPHPHPHPGGQTPEPEDPPGAPHTEEEGAED

SEQ ID NO: 20 - protein, 282 aa, Homo sapiens
MQRLRWLRDWKSSGRGLTAAKEPGARSSPLQAMRILQLILLALATGLVGGETRIIKGFECKPHSQPWQAALFEKTRLLCGATLIAPRWLLTAAHCLKPRYIVHLGQHNLQKEEGCEQTRTATESFPHPGFNNSLPNKDHRNDIMLVKMASPVSITWAVRPLTLSSRCVTAGTSCLISGWGSTSSPQLRLPHTLRCANITIIEHQKCENAYPGNITDTMVCASVQEGGKDSCQGDSGGPLVCNQSLQGIISWGQDPCAITRKPGVYTKVCKYVDWIQETMKNN

SEQ ID NO: 21 - protein, 168 aa, Homo sapiens
MQAQQYQQQRRKFAAAFLAFIFILAAVDTAEAGKKEKPEKKVKKSDCGEWQWSVCVPTSGDCGLGTREGTRTGAECKQTMKTQRCKIPCNWKKQFGAECKYQFQAWGECDLNTALKTRTGSLKRALHNAECQKTVTISKPCGKLTKPKPQAESKKKKKEGKKQEKMLD

SEQ ID NO: 22 - protein, 314 aa, Homo sapiens
MGIGRSEGGRRGAALGVLLALGAALLAVGSASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNATEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFK

SEQ ID NO: 23 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 24 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 25 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 26 - protein, 430 aa, Homo sapiens
MKPSLLCRPLSCFLMLLPWPLATLTSTTLWQCPPGEEPDLDPGQGTLCRPCPPGTFSAAWGSSPCQPHARCSLWRRLEAQVGMATRDTLCGDCWPGWFGPWGVPRVPCQPCSWAPLGTHGCDEWGRRARRGVEVAAGASSGGETRQPGNGTRAGGPEETAAQYAVIAIVPVFCLMGLLGILVCNLLKRKGYHCTAHKEVGPGPGGGGSGINPAYRTEDANEDTIGVLVRLITEKKENAAALEELLKEYHSKQLVQTSHRPVSKLPPAPPNVPHICPHRHHLHTVQGLASLSGPCCSRCSQKKWPEVLLSPEAVAATTPVPSLLPNPTRVPKAGAKAGRQGEITILSVGRFRVARIPEQRTSSMVSEVKTITEAGPSWGDLPDSPQPGLPPEQQALLGSGGSRTKWLKPPAENKAEENRYVVRLSESNLVI

SEQ ID NO: 27 - protein, 146 aa, Homo sapiens
MAGPLRAPLLLLAILAVALAVSPAAGSSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

SEQ ID NO: 28 - protein, 21 aa, Artificial Sequence
  GDF15
GSIYIHVYFTKSGFHPDPRQG

SEQ ID NO: 29 - protein, 21 aa, Artificial Sequence
  GDF15
KALYRRLATVHMSRMINKYKG

SEQ ID NO: 30 - protein, 21 aa, Artificial Sequence
  GDF15
ITINIVDDHTPWVKGSVPPPG

SEQ ID NO: 31 - protein, 21 aa, Artificial Sequence
  GDF15
LDQYVKFDAVSGDYYPIIYFG

SEQ ID NO: 32 - protein, 21 aa, Artificial Sequence
  GDF15
YPINESLASLPLRVSFCPLSG

SEQ ID NO: 33 - protein, 21 aa, Artificial Sequence
  GDF15
GDYYPIIYFNDYWNLQKDYYG

SEQ ID NO: 34 - protein, 20 aa, Artificial Sequence
  GDF15
GSIYIHVYFTKSGFHPDPRQ

SEQ ID NO: 35 - protein, 20 aa, Artificial Sequence
  GDF15
KALYRRLATVHMSRMINKYK

SEQ ID NO: 36 - protein, 20 aa, Artificial Sequence
  GDF15
ITINIVDDHTPWVKGSVPPP

SEQ ID NO: 37 - protein, 20 aa, Artificial Sequence
  GDF15
LDQYVKFDAVSGDYYPIIYF

SEQ ID NO: 38 - protein, 20 aa, Artificial Sequence
  GDF15
YPINESLASLPLRVSFCPLS

SEQ ID NO: 39 - protein, 20 aa, Artificial Sequence
  GDF15
GDYYPIIYFNDYWNLQKDYY